(19) United States
(12) Patent Application Publication
(10) Pub. No.: US 2025/0281424 A1
(43) Pub. Date: Sep. 11, 2025

(54) SMALL MOLECULE ANTAGONISTS OF PF4

(71) Applicants: Yuhang ZHOU, Philadelphia, PA (US); New York Blood Center, Inc, New York, NY (US); Fox Chase Chemical Diversity Center, Inc., Doylestown, PA (US); Thomas Jefferson University, Philadelphia, PA (US)

(72) Inventors: Bruce Sachais, New York, NY (US); Jay E. Wrobel, Lawrenceville, NJ (US); Allen B. Reitz, Lansdale, PA (US); Steven E. McKenzie, Springfield, PA (US); Mark E. McDonnell, Lansdale, PA (US); Haiyan Bian, Princeton, NJ (US); Thomas A. Ford-Hutchinson, Doylestown, PA (US); Robert J. Rosano, Chester Springs, PA (US); Yuhang Zhou, Philadelphia, PA (US); Nandan Mandayam, New York, NY (US); Jennifer Clemens, Doylestown, PA (US); Justin Sausker, Jamison, PA (US); Colin M. Tice, Maple Glen, PA (US)

(21) Appl. No.: 18/247,216

(22) PCT Filed: Sep. 29, 2021
(86) PCT No.: PCT/US2021/071635

§ 371 Date: Mar. 29, 2023

Related U.S. Application Data
(60) Provisional application No. 63/084,699, filed on Sep. 29, 2020.

(51) Int. Cl. A61K 31/122; A61K 31/165; A61K 31/341; A61K 31/35; A61K 31/351; A61K 31/36; A61K 31/397; A61K 31/4025; A61K 31/403; A61K 31/4155; A61K 31/427; A61K 31/44; A61K 31/506; A61K 31/704; A61P 7/02; C07C 49/563; C07C 235/82; C07C 271/06; C07D 207/08; C07D 213/50; C07D 213/75; C07D 231/12; C07D 239/26; C07D 277/04; C07D 307/52; C07D 309/14; C07D 317/58; C07D 319/18; C07H 15/18

(52) U.S. Cl. CPC A61K 31/122; A61K 31/165; A61K 31/341; A61K 31/35; A61K 31/351; A61K 31/36; A61K 31/397; A61K 31/4025; A61K 31/403; A61K 31/4155; A61K 31/427; A61K 31/44; A61K 31/506; A61K 31/704; A61P 7/02; C07C 49/563; C07C 235/82; C07C 271/06; C07D 207/08; C07D 213/50; C07D 213/75; C07D 231/12; C07D 239/26; C07D 277/04; C07D 307/52; C07D 309/14; C07D 317/58; C07D 319/18; C07H 15/18

(57) ABSTRACT

The present application provides a compound of Formula (I) or a pharmaceutically acceptable salt thereof, wherein Y, R¹, R², R³ and R⁴ are described herein. The methods of using these compounds to inhibit tetramerization of PF4 and to treat the associated diseases and conditions, such as heparin-induced thrombocytopenia and thrombosis (HITT) and vaccine-induced immune thrombotic thrombocytopenia (VITT), methods of making these compounds, and pharmaceutical compositions containing these compounds are also disclosed.

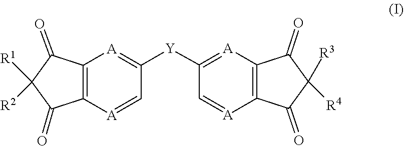

### CROSS REFERENCE TO RELATED APPLICATIONS

The present application claims the benefit of U.S. Provisional patent application 63/084,699 filed Sep. 29, 2021, the entire contents of which is incorporated by reference herein.

### STATEMENT REGARDING FEDERALLY SPONSORED RESEARCH OR DEVELOPMENT

This invention was made with government support under Grant Nos. R41HL123126-01 and R42HL123126-02 awarded by the National Institutes of Health. The government has certain rights in the invention.

### TECHNICAL FIELD

This invention relates to inhibitors of PF4 tetramerization useful in treating, for example, heparin induced thrombocytopenia (HIT) or heparin induced thrombocytopenia and thrombosis (HITT).

### BACKGROUND

Heparin-induced thrombocytopenia and thrombosis (HITT) is a serious complication of heparin therapy. Heparin is a naturally-occurring anticoagulant that prevents the formation of clots and extension of existing clots within the vasculature. Major medical applications of heparin include dialysis, cardiac catheterization, and cardiopulmonary bypass surgery. However, heparin therapy may lead to a serious complication known as heparin induced thrombocytopenia (HIT). HIT is caused by an immunological reaction that targets platelets leading to a low platelet count (thrombocytopenia). HIT increases the risk of blood clots forming within blood vessels and blocking the flow of blood (thrombosis), referred to as HITT when thrombosis occurs. HITT develops in approximately 1-3% of patients treated with heparin for 5-10 days. Affected individuals have a 20-50% risk of developing new thromboembolic events, a mortality rate of about 20%, and an additional ˜10% of patients require amputations or suffer other major morbidity. The rate of occurrence of HITT is about 10-20 cases/yr/hospital, and the patients with this condition are not adequately treated by existing therapies.

Despite the introduction of low molecular weight heparins (LMWH) and the synthetic pentasaccharide fondaparinux, HITT continues to be a significant medical problem. This is likely due to the fact that heparin remains the anticoagulant of choice for many patients (such as patients undergoing cardiopulmonary bypass or percutaneous coronary intervention, at high risk for bleeding, or with renal failure). HITT occurs even after treatment with LMWHs, although at a reduced rate compared to unfractionated heparin in some patients. As a large number of hospitalized patients are exposed to heparin, HITT is a major treatment-induced cause of morbidity and mortality in this patient population.

### SUMMARY

The present application provides compounds that inhibit platelet activation by directly inhibiting tetramerization of platelet factor 4 (PF4), and may be useful in treating diseases and conditions in which increased blood clotting is indicated. Suitable examples of such diseases include HIT and HITT. The compounds of the present application have high potency at a μM level, little or no activity against other chemokines, excellent ADMET properties including microsome stability and little or no cytochrome P450 inhibition, little or no cytotoxicity, little or no hERG inhibition, good aqueous solubility and PK parameters suitable for intravenous (IV) administration, such as moderate-long half-life, and low clearance and volume of distribution. Exemplary embodiments of such compounds are described below.

In some embodiments, the present disclosure provides a compound of Formula (I):

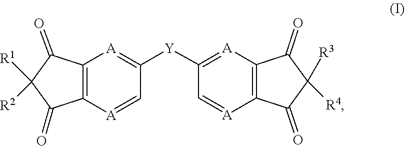

or a pharmaceutically acceptable salt thereof, wherein A, Y, R¹, R², R³ and R⁴ are as described herein.

In some embodiments, the present disclosure provides a pharmaceutical composition comprising a compound of Formula (I), or a pharmaceutically acceptable salt thereof, and a pharmaceutically acceptable carrier.

In some embodiments, the present disclosure provides a method of:
preventing formation of platelet factor-4 (PF4) tetramers in a subject; and/or
disrupting platelet factor-4 (PF4) tetramers in a subject; and/or
preventing formation of an ultra-large complex (ULC) comprising a PF4 tetramer and a glycosaminoglycan (GAG) or other polyanion in a subject; and/or
inhibiting ULC-antibody complex binding to a FcγRIIa receptor on a platelet in a subject; and/or
inhibiting platelet aggregation in a subject, and/or
increasing high density lipoproteins in a subject; and/or
modulating clotting or hemostasis in a subject; and/or
correcting a platelet imbalance in a subject, the method comprising administering to the subject a therapeutically effective amount of a compound of Formula (I), or a pharmaceutically acceptable salt thereof, or a pharmaceutical composition disclosed herein.

In some embodiments, the present disclosure provides a method of treating or preventing a disease or condition selected from:
heparin induced thrombocytopenia and thrombosis (HITT);
a thrombotic complication of HITT;
heparin induced thrombocytopenia (HIT);
vaccine-induced immune thrombotic thrombocytopenia (VITT);
atherosclerosis or atherosclerotic vascular disease;
decrease in platelet production;
inflammation or an inflammatory disease;
antiphospholipid syndrome;
platelet imbalance or insufficiency; and
a clotting or hemostasis disorder
   in a subject, the method comprising administering to the subject a therapeutically effective amount of a compound of Formula (I), or a pharmaceutically acceptable salt thereof, or a pharmaceutical composition disclosed herein.

Certain implementations of the embodiments are described herein.

Unless otherwise defined, all technical and scientific terms used herein have the same meaning as commonly understood by one of ordinary skill in the art to which the present application belongs. Methods and materials are described herein for use in the present application; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the present application will be apparent from the following detailed description and figures, and from the claims.

### DESCRIPTION OF DRAWINGS

FIG. 1A is a diagram showing formation of Ultra Large Complex (ULC) of PF4 tetramers and heparin. The figure shows that formation of ULC requires tetramerization of PF4.

FIG. 1B is a diagram showing that inhibitors of tetramerization inhibit ULC formation.

FIG. 2 is a diagram showing simplified mechanism of heparin-induced thrombocytopenia and thrombosis (HITT) pathophysiology.

FIG. 3 is an image showing PF4 dimer with a hypothesized site of binding for PF4 tetramerization antagonists.

FIG. 4 is a graph showing that a compound of Example 1 reduces heparin-PF4-KKO induced thrombocytopenia in vivo in a mouse model of HIT.

FIG. 5 is a line plot showing plasma concentration time curve for the compound of Example 1 given at 1 mg/kg IV bolus to CD-1 mice.

FIG. 6A is a line plot showing dose-dependent inhibition of PF4 tetramerization for compound of Example 3.

FIG. 6B is a line plot showing dose-dependent inhibition of ULC formation for compound of Example 3.

FIG. 7A is a scheme showing equilibrium between tautomeric forms of a compound containing an indane-1,3-dione moiety substituted in the 2-position.

FIG. 7B is a scheme showing equilibrium between tautomeric forms of a compound containing an indane-1,3-dione moiety that is unsubstituted in the 2-position.

FIG. 8 is a bar graph showing inhibition of PF4-KKO induced platelet activation using P-selectin as a marker by the exemplified compounds at 20 μM concentration.

FIG. 9 is a bar graph showing inhibition PF4-KKO induced platelet activation using P-selectin as a marker by the exemplified compounds at 40 μM concentration.

FIG. 10 is a graph showing difference in PF4-KKO induced platelet activation using P-selectin as a marker in human platelets treated with PF4 along and with PF4 and heparin.

FIG. 11 is a graph showing difference in PF4-KKO induced platelet activation using P-selectin as a marker in human platelets treated with PF4 at 37 μg/ml and with PF4 at 65 μg/ml.

FIG. 12 is a graph showing efficacy of compound of Example 15 in preventing PF4-KKO induced platelet activation using P-selectin as a marker in human platelets.

FIG. 13 is a graph showing efficacy of compound of Example 15 in preventing PF4-KKO induced platelet activation using P-selectin as a marker in human platelets.

FIG. 14 is a graph showing efficacy of compound of Example 33 in preventing PF4-KKO induced platelet activation using P-selectin as a marker in FcgRIIA transgenic murine platelets.

FIG. 15 is a graph showing efficacy of compound of Example 34 in preventing PF4-KKO induced platelet activation using P-selectin as a marker in FcgRIIA transgenic murine platelets.

FIG. 16 is a graph showing efficacy of compound of Example 22 in preventing PF4-KKO induced platelet activation using P-selectin as a marker in FcgRIIA transgenic murine platelets.

FIG. 17 is a graph showing efficacy of compound of Example 32 in preventing PF4-KKO induced platelet activation using P-selectin as a marker in FcgRIIA transgenic murine platelets.

FIG. 18 is a graph showing efficacy of compound of Example 28 in preventing PF4-KKO induced platelet activation using P-selectin as a marker in FcgRIIA transgenic murine platelets.

FIG. 19 is a graph showing efficacy of compound of Example 36 in preventing PF4-KKO induced platelet activation using P-selectin as a marker in FcgRIIA transgenic murine platelets.

FIG. 20 is a graph showing efficacy of compound of Example 31 in preventing PF4-KKO induced platelet activation using P-selectin as a marker in FcgRIIA transgenic murine platelets.

FIG. 21 is a graph showing efficacy of compound of Example 29 in preventing PF4-KKO induced platelet activation using P-selectin as a marker in FcgRIIA transgenic murine platelets.

FIG. 22 is a bar graph showing levels of normalized P-selectin representing PF4-KKO induced platelet activation and the inhibition P-selectin levels by Examples 31, 29 33, 34, 22, 28, 36 and 32.

### DETAILED DESCRIPTION

The clinical manifestations of HITT are caused by antibodies that recognize a complex composed of heparin and tetramers of platelet factor 4 (PF4). PF4 is a 70 amino acid, lysine-rich 7.8 kDa platelet-specific protein that belongs to the CXC (or beta) chemokine subfamily. PF4 is synthesized by megakaryocytes and comprises 2-3% of the total released protein in mature platelets. PF4 exists as a tetramer in the α-granules of platelets and is secreted in high concentrations when platelets are activated. PF4 tetramers bind avidly to glycosaminoglycans (GAGs). The interaction of PF4 with GAGs, including heparin, contributes to the pathogenesis of HITT with the formation of ultra large complexes of PF4 tetramers and heparin (ULCs) representing the major antigen recognized by pathogenic HITT antibodies. A transgenic mouse model of HITT demonstrates that heparin, PF4 (forming ULC), anti-heparin/PF4 antibody, and the platelet receptor FcγRIIa are necessary and sufficient to recapitulate the salient features of HITT in vivo. Treatment of patients with heparin is thought to favor the formation of the ULCs, placing these patients at risk for HITT.

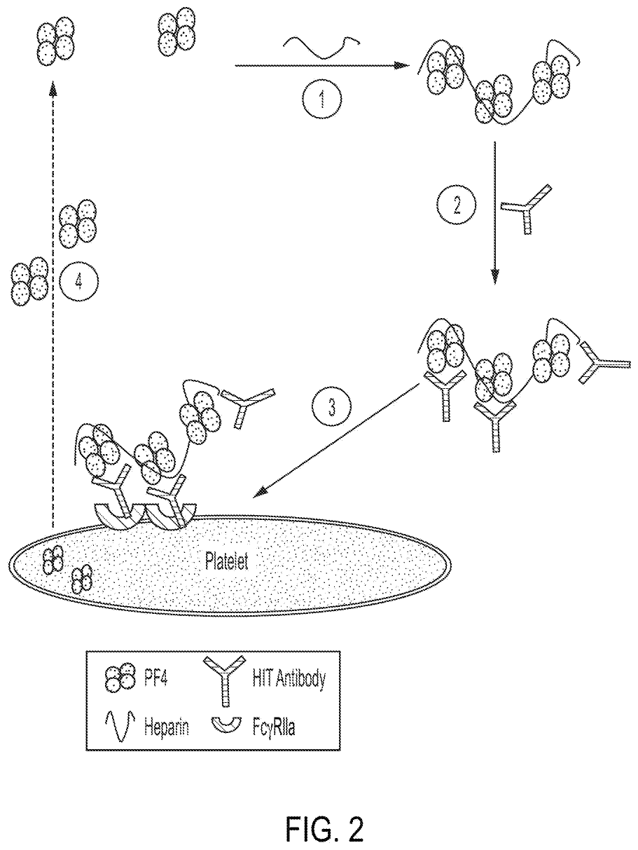

A simplified mechanism of HITT pathophysiology is shown in FIG. 2, and demonstrates the feed-forward nature of this disorder. Specifically, antibody recognition of ULC leads to platelet activation, releasing more PF4, which can form additional ULC to be recognized by antibody. The compounds of the present application inhibit cellular activation by limiting the ULC formation. Referring to FIG. 2, in step (1) PF4 released from activated platelets as a tetramer forms a complex (ULC) with heparin which has been administered to a patient as an anticoagulant; in step (2) pathogenic antibodies bind to the complex of heparin and PF4 tetramer; in step (3) an antibody-decorated heparin-PF4 complex binds to a platelet via its FcγRIIa receptor; and in step (4) crosslinking of FcγRIIa leads to platelet activation and release of additional PF4, which can bind to heparin and feed the pathogenic cycle.

No specific treatments for heparin-induced thrombosis currently exist. For example, current treatment for HITT relies on removal of all heparin exposure from patients with suspected HITT and administration of a non-heparin alternative anticoagulant, typically a direct thrombin inhibitor, which may carry a significant risk of bleeding. Hence, despite discontinued heparin, the patients remain at significant risk for thrombosis and death.

Also disclosed herein are methods of treating thrombosis induced by a vaccine. Vaccine-induced immune thrombotic thrombocytopenia (VITT) is induced in otherwise healthy individuals after an adenovirus-based vaccine and is characterized by development of thrombocytopenia and thrombosis in atypical locations (including, but not limited to, the cerebral and/or splanchic veins) within weeks of receiving a vaccination. VITT has most recently been identified in subject receiving adenovirus-based SARS-CoV-2 vaccines, which involves, in part, antibodies directed toward PF4 and/or PF4-heparin complexes.

Accordingly, the present application provides compounds that directly target and intervene in the pathophysiology of thrombosis, and limit and/or prevent the complications of this condition and related diseases. Exemplary embodiments of the compounds, and methods of making and using these compositions, are described below.

### Definitions

The term “PF4” as used herein refers to platelet factor 4 which is a 70 amino acid, lysine-rich, 7.8 kDa platelet-specific protein that belongs to the CXC (or beta) chemokine subfamily, in which the first two of the four conserved cysteine residues are separated by one amino acid residue. In some embodiments, PF4 is naturally occurring, i.e., wild-type. In other embodiments, PF4 may be synthesized by recombinant or chemical methods. The term PF4 also refers to mutations thereof in which one or more of the amino acids is replaced with a different amino acid. Examples of PF4 mutations are described in International Patent Publication No. WO 02/006300 and the inventors' prior publication WO2013/142328, which is incorporated herein by reference.

As used herein, the term “compound” is meant to include all stereoisomers, geometric isomers, tautomers, and isotopes of the structures named or depicted. Compounds herein identified by name or structure as one particular tautomeric form are intended to include other tautomeric forms unless otherwise specified.

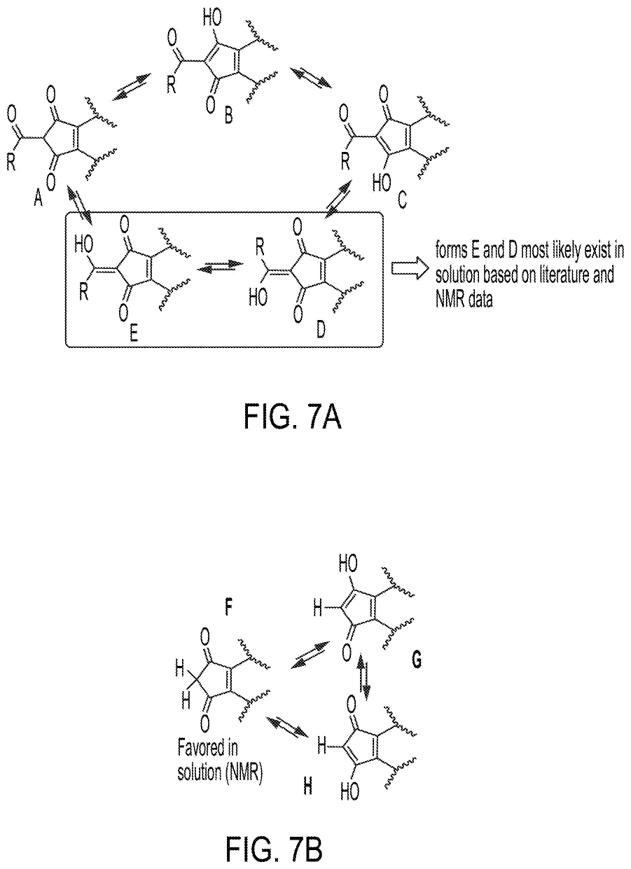

As used herein, the term “tautomer” refers to compounds which are capable of existing in a state of equilibrium between two isomeric forms. Such compounds may differ in the bond connecting two atoms or groups and the position of these atoms or groups in the compound. Suitable examples of an equilibrium between the tautomeric forms are depicted in FIGS. 7A and 7B.

Referring to FIG. 7A, an indane-1,3-dione moiety having an R(C═O)— substituent in the 2-position may exist as an equilibrium between forms A, B, C, D and E. In this example, the R— group may correspond to C₁₋₆ alkoxy, C₁₋₆ alkyl or an NH(R^{a1}) fragment, as in any one of the substituents R¹-R⁴ in a compound of Formula (I) described herein. Based on the analysis of literature and NMR data presented herein, a compound containing an R(C═O)— substituent as depicted in FIG. 7A most likely exists in solution as an equilibrium between forms E and D. See, e.g., Liepins̆, E. et al. Magnetic Resonance in Chemistry 1989, 27:907; Song, J. et al. Organic Letters 2007, 9, 4307; and Paul, B. K.; Guchhait, N. Computational and Theoretical Chemistry 2013, 1012, 2. Referring to FIG. 7B, an indane-1,3-dione moiety that is unsubstituted in the 2-position may exist as an equilibrium between forms F, G and H. This is the case of a compound of Formula (I) described herein when either R¹ and R² are both H, or R³ and R⁴ are both H, or R¹—R⁴ are all H. Based on the analysis of NMR data, form F is favored in solution of such a compound.

As used herein, the term “isomer” refers to structural, geometric and stereo isomers. As the compound of the present application may have one or more chiral centers, it is capable of existing in enantiomeric forms.

As used herein, the phrase “optionally substituted” means unsubstituted or substituted. As used herein, the term “substituted” means that a hydrogen atom is removed and replaced by a substituent. It is to be understood that substitution at a given atom is limited by valency.

As used in the present application, the term “Cₙ₋ₘ alkyl”, employed alone or in combination with other terms, refers to a saturated hydrocarbon group that may be straight-chain (linear) or branched, having n to m carbons. Examples of alkyl moieties include, but are not limited to, chemical groups such as methyl, ethyl, n-propyl, isopropyl, n-butyl, tert-butyl, isobutyl, sec-butyl; higher homologs such as 2-methyl-1-butyl, n-pentyl, 3-pentyl, n-hexyl, 1,2,2-trimethylpropyl, and the like. In some embodiments, the alkyl group contains from 1 to 6 carbon atoms, from 1 to 4 carbon atoms, from 1 to 3 carbon atoms, or 1 to 2 carbon atoms.

As used in the present application, “Cₙ₋ₘ alkenyl” refers to an alkyl group having one or more double carbon-carbon bonds and having n to m carbons. Example alkenyl groups include, but are not limited to, ethenyl, n-propenyl, isopropenyl, n-butenyl, sec-butenyl, and the like. In some embodiments, the alkenyl moiety contains 2 to 6, 2 to 4, or 2 to 3 carbon atoms.

As used in the present application, “Cₙ₋ₘ alkylidene” refers to a divalent functional group derived from an alkane by removal of two hydrogen atoms from the same carbon atom, the free valencies being part of a double bond. Suitable examples of alkylidene include CH₂═ (methylene), CH₃CH═, and (CH₃)₂C═.

As used in the present application, the term “Cₙ₋ₘ alkoxy”, employed alone or in combination with other terms, refers to a group of formula —O—Cₙ₋ₘ alkyl, win the present application the alkyl group contains n to m carbon atoms. Examplary alkoxy groups include, but are not limited to, methoxy, ethoxy, propoxy (for example, n-propoxy and isopropoxy), butoxy (for example, n-butoxy and tert-butoxy), and the like. In some embodiments, the alkoxy group has 1 to 6, 1 to 4, or 1 to 3 carbon atoms.

As used in the present application, “halo” refers to a halogen atom such as F, Cl, Br, or I. In some embodiments, a halo is F, Cl, or Br. In other embodiments, halo is F, Cl, or I. In other embodiments, halo is F, I, or Br.

As used in the present application, the term “Cₙ₋ₘ haloalkyl”, employed alone or in combination with other terms, refers to an alkyl group having from one halogen atom to 2s+1 halogen atoms which may be the same or different, where “s” is the number of carbon atoms in the alkyl group, win the present application the alkyl group has n to m carbon atoms. In some embodiments, the haloalkyl group is fluorinated only. In some embodiments, the alkyl group has 1 to 6, 1 to 4, or 1 to 3 carbon atoms.

As used in the present application, “Cₙ₋ₘ haloalkoxy” refers to a group of formula —O-haloalkyl having n to m carbon atoms. An example haloalkoxy group is OCF₃. In some embodiments, the haloalkoxy group is fluorinated only. In some embodiments, the alkyl group has 1 to 6, 1 to 4, or 1 to 3 carbon atoms.

As used in the present application, “cycloalkyl” refers to non-aromatic saturated or unsaturated cyclic hydrocarbons including cyclized alkyl and/or alkenyl groups. Cycloalkyl groups can include mono- or polycyclic (for example, having 2, 3 or 4 fused rings) groups and spirocycles. Ring-forming carbon atoms of a cycloalkyl group can be optionally substituted by oxo or sulfido (for example, C(O) or C(S)). Also included in the definition of cycloalkyl are moieties that have one or more aromatic rings fused (that is, having a bond in common with) to the non-aromatic cyclic hydrocarbon, for example, benzo or thienyl derivatives of cyclopentane, cyclohexane, and the like. A cycloalkyl group containing a fused aromatic ring can be attached through any ring-forming atom including a ring-forming atom of the fused aromatic ring. Cycloalkyl groups can have 3, 4, 5, 6, 7, 8, 9, 10, 11, or 12 ring-forming atoms. In some embodiments, the cycloalkyl is a 3-12 membered monocyclic or bicyclic cycloalkyl. In some embodiments, the cycloalkyl is a C₃₋₇ monocyclic cycloalkyl. Examples of cycloalkyl groups include cyclopropyl, cyclobutyl, cyclopentyl, cyclohexyl, cycloheptyl, cyclopentenyl, cyclohexenyl, cyclohexadienyl, cycloheptatrienyl, norbornyl, norpinyl, norcarnyl, cyclooctyl, cyclooctenyl, and the like. In some embodiments, cycloalkyl is cyclopropyl, cyclobutyl, cyclopentyl, cyclohexyl, cyclooctyl, or cyclooctenyl. In some embodiments, the cycloalkyl is a cyclooctenyl ring fused with 1 or 2 benzene rings. In some embodiments, the cycloalkyl is a 3-8 membered or 3-7 membered monocyclic cycloalkyl group (for example, C₃₋₈ or C₃₋₇ cycloalkyl). In some embodiments, the cycloalkyl is a 8-12-membered bicyclic cycloalkyl. In some embodiments, the cycloalkyl is a 8-16-membered bicyclic or tricyclic cycloalkyl (for example, C₈₋₁₆ cycloalkyl). In some embodiments, the cycloalkyl is unsaturated cyclic hydrocarbon group (that is, the cycloalkyl contains at least one double bond).

As used herein, “heteroalkyl” refers to branched or unbranched heteroalkyls having one or more heteroatoms selected, independently, from O, N, or S. Examples of heteroalkyls include, but are not limited to, CH₂CH₂OCH₂CH₂OCH₂CH₂OCH₃, CH₂CH₂OCH₂CH₂OCH₃, CH₂NHCH₂CH₂OCH₂CH₂OCH₃, and CH₂CH₂CH₂NH₂, and the like.

As used herein, “heterocycloalkyl” or “aliphatic heterocycle” refers to non-aromatic saturated or unsaturated monocyclic or polycyclic heterocycles having one or more ring-forming heteroatoms selected from O, N, or S. Included in heterocycloalkyl are monocyclic 4-, 5-, 6-, 7-, 8-, 9- or 10-membered heterocycloalkyl groups. Heterocycloalkyl groups can also include spirocycles. Example heterocycloalkyl groups include pyrrolidin-2-one, 1,3-isoxazolidin-2-one, pyranyl, tetrahydropuran, oxetanyl, azetidinyl, morpholino, thiomorpholino, piperazinyl, tetrahydrofuranyl, tetrahydrothienyl, piperidinyl, pyrrolidinyl, isoxazolidinyl, isothiazolidinyl, pyrazolidinyl, oxazolidinyl, thiazolidinyl, imidazolidinyl, azepanyl, benzazapene, and the like. Ring-forming carbon atoms and heteroatoms of a heterocycloalkyl group can be optionally substituted by oxo or sulfido groups (e.g., C(O), S(O), C(S), or S(O)2, etc.). The heterocycloalkyl group can be attached through a ring-forming carbon atom or a ring-forming heteroatom. In some embodiments, the heterocycloalkyl group contains 0 to 3 double bonds. In some embodiments, the heterocycloalkyl group contains 0 to 2 double bonds. In some embodiments, the heterocycloalkyl group is unsaturated (i.e., the heterocycloalkyl contains at least one double bond). Also included in the definition of heterocycloalkyl are moieties that have one or more aromatic rings fused (i.e., having a bond in common with) to the non-aromatic heterocycle, for example, benzo or thienyl derivatives of piperidine, morpholine, azepine, etc. A heterocycloalkyl group containing a fused aromatic ring can be attached through any ring-forming atom including a ring-forming atom of the fused aromatic ring. In some embodiments, the heterocycloalkyl is a monocyclic 4-6 membered heterocycloalkyl having 1 or 2 heteroatoms independently selected from nitrogen, oxygen, or sulfur and having one or more oxidized ring members. In some embodiments, the heterocycloalkyl is a monocyclic or bicyclic 4-10 membered heterocycloalkyl having 1, 2, 3, or 4 heteroatoms independently selected from nitrogen, oxygen, or sulfur and having one or more oxidized ring members. In some embodiments, the heterocycloalkyl is a 8-12-membered heterocycloalkyl (e.g., bicyclic heterocycloalkyl). In some embodiments, the heterocycloalkyl is a 8-16-membered heterocycloalkyl (e.g., bicyclic or tricyclic heterocycloalkyl). In some embodiments, the 8-12 membered bicyclic heterocycloalkyl is a 8-12 membered fused heterocycloalkylaryl group or a 8-12 membered fused heterocycloalkylheteroaryl group. In some embodiments, the heterocycloalkyl is a 9-12 membered bicyclic heterocycloalkyl. In some embodiments, the 9-10 membered bicyclic heterocycloalkyl is a 9-10 membered fused heterocycloalkylaryl group or a 9-10 membered fused heterocycloalkylheteroaryl group. The term “heterocycloalkylene” refers to a divalent heterocycloalkyl linking group.

As used in the present application, “heteroaryl” refers to a monocyclic or polycyclic aromatic heterocycle having at least one heteroatom ring member selected from sulfur, oxygen, and nitrogen. In some embodiments, the heteroaryl ring has 1, 2, 3, or 4 heteroatom ring members independently selected from nitrogen, sulfur and oxygen. In some embodiments, any ring-forming N in a heteroaryl moiety can be an N-oxide. In some embodiments, the heteroaryl is a 5-10 membered monocyclic or bicyclic heteroaryl having 1, 2, 3 or 4 heteroatom ring members independently selected from nitrogen, sulfur and oxygen. In some embodiments, the heteroaryl is a 5-6 membered monocyclic heteroaryl having 1 or 2 heteroatom ring members independently selected from nitrogen, sulfur and oxygen. In some embodiments, the heteroaryl is a five-membered or six-membered heteroaryl ring. A five-membered heteroaryl ring is a heteroaryl with a ring having five ring atoms win the present application one or more (for example, 1, 2, or 3) ring atoms are independently selected from N, O, and S. Exemplary five-membered heteroaryls are thienyl, furyl, pyrrolyl, imidazolyl, thiazolyl, oxazolyl, pyrazolyl, isothiazolyl, isoxazolyl, 1,2,3-triazolyl, tetrazolyl, 1,2,3-thiadiazolyl, 1,2,3-oxadiazolyl, 1,2,4-triazolyl, 1,2,4-thiadiazolyl, 1,2,4-oxadiazolyl, 1,3,4-triazolyl, 1,3,4-thiadiazolyl, and 1,3,4-oxadiazolyl. A six-membered heteroaryl ring is a heteroaryl with a ring having six ring atoms win the present application one or more (for example, 1, 2, or 3) ring atoms are independently selected from N, O, and S. Exemplary six-membered heteroaryls are pyridyl, pyrazinyl, pyrimidinyl, triazinyl and pyridazinyl.

The term “aromatic” refers to a carbocycle or heterocycle having one or more polyunsaturated rings having aromatic character (that is, having (4n+2) delocalized π (pi) electrons where n is an integer).

The term “n-membered” where n is an integer, typically describes the number of ring-forming atoms in a moiety where the number of ring-forming atoms is n. For example, piperidinyl is an example of a 6-membered heterocycloalkyl ring, pyrazolyl is an example of a 5-membered heteroaryl ring, pyridyl is an example of a 6-membered heteroaryl ring, and 1,2,3,4-tetrahydro-naphthalene is an example of a 10-membered cycloalkyl group.

The term “aryl,” employed alone or in combination with other terms, refers to an aromatic hydrocarbon group, which may be monocyclic or polycyclic (for example, having 2, 3 or 4 fused rings). The term “Cₙ₋ₘ aryl” refers to an aryl group having from n to m ring carbon atoms. Aryl groups include, for example, phenyl, naphthyl, anthracenyl, phenanthrenyl, indanyl, indenyl and the like. In some embodiments, aryl groups have from 6 to about 20 carbon atoms, from 6 to about 15 carbon atoms, or from 6 to about 10 carbon atoms. In some embodiments, the aryl group is phenyl.

As used in the present application, the term “Cₙ₋ₘ alkoxycarbonyl” refers to a group of formula —C(O)O-alkyl, wherein the alkyl group has n to m carbon atoms. In some embodiments, the alkyl group has 1 to 6, 1 to 4, or 1 to 3 carbon atoms. Examples of alkoxycarbonyl groups include, but are not limited to, methoxycarbonyl, ethoxycarbonyl, propoxycarbonyl (for example, n-propoxycarbonyl and isopropoxycarbonyl), butoxycarbonyl (for example, n-butoxycarbonyl and tert-butoxycarbonyl), and the like.

As used in the present application, the term “Cₙ₋ₘ alkylcarbonyl” refers to a group of formula —C(O)-alkyl, win the present application the alkyl group has n to m carbon atoms. In some embodiments, the alkyl group has 1 to 6, 1 to 4, or 1 to 3 carbon atoms. Examples of alkylcarbonyl groups include, but are not limited to, methylcarbonyl, ethylcarbonyl, propylcarbonyl (for example, n-propylcarbonyl and isopropylcarbonyl), butylcarbonyl (for example, n-butylcarbonyl and tert-butylcarbonyl), and the like.

As used in the present application, the term “carboxy” or “carboxyl” refers to a —C(O)OH group.

The terms “pharmaceutical” and “pharmaceutically acceptable” are employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “cell” is meant to refer to a cell that is in vitro, ex vivo or in vivo. In some embodiments, an ex vivo cell can be part of a tissue sample excised from an organism such as a mammal. In some embodiments, an in vitro cell can be a cell in a cell culture. In some embodiments, an in vivo cell is a cell living in an organism such as a mammal.

As used herein, the term “individual”, “patient”, or “subject” used interchangeably, refers to any animal, including mammals, preferably mice, rats, other rodents, rabbits, dogs, cats, swine, cattle, sheep, horses, or primates, and most preferably humans.

As used herein, the phrase “effective amount” or “therapeutically effective amount” refers to the amount of active compound or pharmaceutical agent that elicits the biological or medicinal response in a tissue, system, animal, individual or human that is being sought by a researcher, veterinarian, medical doctor or other clinician.

As used herein the term “treating” or “treatment” refers to 1) inhibiting the disease; for example, inhibiting a disease, condition or disorder in an individual who is experiencing or displaying the pathology or symptomatology of the disease, condition or disorder (i.e., arresting further development of the pathology and/or symptomatology), or 2) ameliorating the disease; for example, ameliorating a disease, condition or disorder in an individual who is experiencing or displaying the pathology or symptomatology of the disease, condition or disorder (i.e., reversing the pathology and/or symptomatology).

As used herein, the term “preventing” or “prevention” of a disease, condition or disorder refers to decreasing the risk of occurrence of the disease, condition or disorder in a subject or group of subjects (e.g., a subject or group of subjects predisposed to or susceptible to the disease, condition or disorder). In some embodiments, preventing a disease, condition or disorder refers to decreasing the possibility of acquiring the disease, condition or disorder and/or its associated symptoms. In some embodiments, preventing a disease, condition or disorder refers to completely or almost completely stopping the disease, condition or disorder from occurring.

As used herein, in such methods the term “biological sample” refers to a body fluid or tissue. The body fluid can include, without limitation, whole blood, serum, plasma, peripheral blood, synovial fluid, cerebrospinal fluid, saliva, urine, semen, or other fluid secretion. The term “tissue” can include, without limitation, bone marrow and lymph node, as well as samples of other tissues.

### Therapeutic Compounds

The present application provides, inter alia, a compound of Formula (I-1):

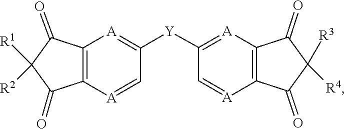

or a pharmaceutically acceptable salt thereof, wherein:
A is selected from CH or N;
Y is selected from O, C(═O), S(═O)₂, C(R⁵)(R⁶), C≡C, or a bond between the indane-1,3-dione rings of the compound of Formula (I);
R¹ and R³ are each independently selected from C₁₋₆ heteroalkyl, H, halo, CN, C₁₋₆ alkoxycarbonyl, C₁₋₆ alkylcarbonyl, C(O)NH(R^{a1}), and C(O)Cy⁴, wherein C₁₋₆ alkyl in the C₁₋₆ alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl, C₁₋₃ alkoxy-C₁₋₃ alkoxy, carboxy, C₁₋₃alkoxycarbonyl, C₆₋₁₀ aryloxy, —N(C₁₋₃ alkyl)(C₁₋₆alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo, wherein said C₁₋₆ heteroalkyl is optionally substituted with C(O)O—(C₁₋₆ alkyl);
R² and R⁴ are each independently selected from H, C₁₋₆ alkoxycarbonyl, and C₁₋₆ alkylcarbonyl;
R⁵ and R⁶ are each C₁₋₃ haloalkyl;
each R^{a1} is independently selected from C₁₋₆ heteroalkyl, C₁₋₆ alkyl, C₁₋₆ alkenyl, Cy¹, C₁₋₆alkoxycarbonyl, and S(O)₂R^{a2}, wherein said C₁₋₆ alkyl is optionally substituted with 1, 2, or 3 substituents independently selected from Cy², carboxyl, C₁₋₃ alkoxycarbonyl, C₁₋₃ alkoxy, C₁₋₃ alkoxy-C₁₋₃ alkoxy, and C₁₋₃ haloalkoxy, wherein said C₁₋₆ heteroalkyl is optionally substituted with C(O)O—(C₁₋₆ alkyl);
each Cy¹ is independently selected from oxo, S(O₂)—(C₁₋₆ alkyl), C₃₋₁₀ cycloalkyl, 4-10 membered heterocycloalkyl, C₆₋₁₀ aryl, and 5-10 membered heteroaryl, each of which is optionally substituted with 1, 2, or 3 substituents independently selected from halo, NO₂, C₁₋₆ alkyl, C₁₋₃ alkoxy, C₁₋₆ alkoxycarbonyl, C₁₋₆ alkylcarbonyl, and Cy³⁻;
each Cy² is independently selected from oxo, S(O₂)—(C₁₋₆ alkyl), C₆₋₁₀ aryl, C₃₋₁₀ cycloalkyl, 4-10 membered heterocycloalkyl, and 5-10 membered heteroaryl, each of which is optionally substituted with 1, 2, or 3 substituents independently selected from halo, C₁₋₃ alkyl, C₁₋₃ alkoxy, and C₁₋₃ haloalkoxy;
each Cy³ is independently selected from C₆₋₁₀ aryl and 5-10 membered heteroaryl;
each Cy⁴ is independently selected from C₆₋₁₀ aryl, C₃₋₁₀ cycloalkyl, 4-10 membered heterocycloalkyl, and 5-10 membered heteroaryl, each of which is optionally substituted with 1, 2, or 3 substituents independently selected from halo, aryl, carboxy, C₁₋₆ alkyl, C₁₋₆alkoxy, C₁₋₆ alkoxycarbonyl, C₁₋₆ alkylidene, and oxo; wherein C₁₋₆ alkoxy in said C₁₋₆ alkoxycarbonyl is optionally substituted with C₆₋₁₀ aryl; and
each R^{a2} is C₆₋₁₀ aryl, optionally substituted with C₁₋₃ alkyl.

The present application also provides, inter alia, a compound of Formula (I):

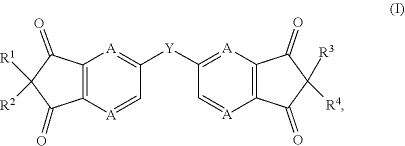

or a pharmaceutically acceptable salt thereof, wherein:
A is selected from CH or N;
Y is selected from O, C(═O), S(═O)₂, C(R⁵)(R⁶), C≡C, and a bond between the indane-1,3-dione rings of the compound of Formula (I);
R¹ and R³ are each independently selected from H, halo, CN, C₁₋₆ alkoxycarbonyl, C₁₋₆ alkylcarbonyl, C(O)NH(R^{a1}), and C(O)Cy⁴, wherein C₁₋₆ alkyl in the C₁₋₆ alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl, C₁₋₃ alkoxy-C₁₋₃ alkoxy, carboxy, C₁₋₃ alkoxycarbonyl, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl; wherein C₆₋₁₀ arylcarbonyl may be optionally substituted with 1, 2, or 3 substituents independently selected from C₁₋₃ alkyl and halo;
R² and R⁴ are each independently selected from H, C₁₋₆ alkoxycarbonyl, and C₁₋₆ alkylcarbonyl;
R⁵ and R⁶ are each C₁₋₃ haloalkyl;
each R^{a1} is independently selected from C₁₋₆ alkyl, C₁₋₆ alkenyl, Cy¹, C₁₋₆ alkoxycarbonyl, and S(O)₂R^{a2}, wherein said C₁₋₆ alkyl is optionally substituted with 1, 2, or 3 substituents independently selected from Cy², carboxyl, C₁₋₃ alkoxycarbonyl, C₁₋₃ alkoxy, C₁₋₃ alkoxy-C₁₋₃ alkoxy, and C₁₋₃ haloalkoxy;
each Cy¹ is independently selected from C₃₋₁₀ cycloalkyl, 4-10 membered heterocycloalkyl, C₆₋₁₀ aryl, and 5-10 membered heteroaryl, each of which is optionally substituted with 1, 2, or 3 substituents independently selected from halo, NO₂, C₁₋₆ alkyl, C₁₋₃ alkoxy, C₁₋₆ alkoxycarbonyl, C₁₋₆ alkylcarbonyl, and Cy³;
each Cy² is independently selected from C₆₋₁₀ aryl, C₃₋₁₀ cycloalkyl, 4-10 membered heterocycloalkyl, and 5-10 membered heteroaryl, each of which is optionally substituted with 1, 2, or 3 substituents independently selected from halo, C₁₋₃ alkyl, C₁₋₃ alkoxy, and C₁₋₃ haloalkoxy;
each Cy³ is independently selected from C₆₋₁₀ aryl and 5-10 membered heteroaryl;
each Cy⁴ is independently selected from C₆₋₁₀ aryl, C₃₋₁₀ cycloalkyl, 4-10 membered heterocycloalkyl, and 5-10 membered heteroaryl, each of which is optionally substituted with 1, 2, or 3 substituents independently selected from halo, aryl, carboxy, C₁₋₆ alkyl, C₁₋₆ alkoxy, C₁₋₆ alkoxycarbonyl, C₁₋₆ alkylidene, and oxo; wherein C₁₋₆ alkoxy in said C₁₋₆ alkoxycarbonyl is optionally substituted with C₆₋₁₀ aryl; and
each R^{a2} is C₆₋₁₀ aryl, optionally substituted with C₁₋₃ alkyl.

In some embodiments of the formulae provided herein, Y is O.

In some embodiments, Y is C(═O).

In some embodiments, Y is S(═O)₂.

In some embodiments, Y is C(R⁵)(R⁶).

In some embodiments, R⁵ is CF₃ and R⁶ is CF₃.

In some embodiments, Y is C(CF₃)₂.

In some embodiments, Y is a bond between the indane-1,3-dione rings of the compound of Formula (I).

In some embodiments, Y is C≡C.

In some embodiments, Y is selected from O, C(═O), S(═O)₂ and C(R⁵)(R⁶).

In some embodiments, Y is selected from C(═O), S(═O)₂ and C(R⁵)(R⁶).

In some embodiments, Y is selected from O, C(═O), S(═O)₂ and C(CF₃)₂.

In some embodiments, Y is selected from O, C(═O), and S(═O)₂.

In some embodiments, Y is selected from C(═O), S(═O)₂ and C(CF₃)₂.

In some embodiments, Y is selected from C(═O) and S(═O)₂.

In some embodiments, Y is selected from O and C(═O).

In some embodiments, Y is selected from O and S(═O)₂.

In some embodiments, Y is selected from O and C(CF₃)₂.

In some embodiments, Y is selected from O and C(═O), R¹ is C(O)NH(R^{a1}); and R³ is selected from H, C₁₋₆ alkoxycarbonyl, C₁₋₆ alkylcarbonyl, and C(O)NH(R^{a1}).

In some embodiments, Y is selected from C(═O) and S(═O)₂. In some aspects of these embodiments, R¹ is C(O)NH(R^{a1}); and R³ is selected from H, C₁₋₆ alkoxycarbonyl, C₁₋₆ alkylcarbonyl, and C(O)NH(R^{a1}).

In some embodiments, R¹ is selected from: C₁₋₆ alkoxycarbonyl, C₁₋₆ alkylcarbonyl, C(O)NH(R^{a1}), and C(O)Cy⁴, wherein C₁₋₆ alkyl in the C₁₋₆ alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl, C₁₋₃ alkoxy-C₁₋₃ alkoxy, carboxy, C₆₋₁₀ aryloxy, —N(C₁₋₃ alkyl) (C₁₋₆ alkoxycarbonyl), —NH(C₁₋₆ alkoxycarbonyl), and —NH(C₁₋₆ alkylcarbonyl), and wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R¹ is selected from: C₁₋₆ alkylcarbonyl, C(O)NH(R^{a1}), and C(O)Cy⁴, wherein C₁₋₆ alkyl in the C₁₋₆ alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl, C₁₋₃ alkoxy-C₁₋₃ alkoxy, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R¹ is H.

In some embodiments, R¹ is C₁₋₆ alkoxycarbonyl (e.g., ethoxycarbonyl, t-bytoxycarbonyl, isopropoxycarbonyl).

In some embodiments, R¹ is C₁₋₆alkylcarbonyl (e.g., methylcarbonyl, ethyl carbonyl, propylcarbonyl, butylcarbonyl, pentylcarbonyl, or hexylcarbonyl).

In some embodiments, R¹ is C(O)NH(R^{a1}).

In some embodiments, R¹ is C(O)Cy⁴.

In some embodiments, R¹ is C₁₋₆ alkylcarbonyl, wherein C₁₋₆ alkyl in the C₁₋₆ alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl, C₁₋₃ alkoxy-C₁₋₃ alkoxy, carboxy, C₁₋₃alkoxycarbonyl, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R¹ is C₁₋₆ alkylcarbonyl, wherein C₁₋₆ alkyl in the C₁₋₆ alkylcarbonyl is optionally substituted with Cy⁴.

In some embodiments, R¹ is C₁₋₆ alkylcarbonyl, wherein C₁₋₆ alkyl in the C₁₋₆ alkylcarbonyl is optionally substituted with 1, 2, or 3 substituents independently selected from amino, C₁₋₆ alkoxy, C₁₋₆ cycloalkoxy, C₆₋₁₀ arylcarbonyl, C₁₋₃ alkoxy-C₁₋₃ alkoxy, carboxy, C₁₋₃ alkoxycarbonyl, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R¹ is C₁₋₆ alkylcarbonyl, wherein C₁₋₆ alkyl in the C₁₋₆ alkylcarbonyl is optionally substituted with 1, 2, or 3 substituents independently selected from amino, C₁₋₆ alkoxy, C₁₋₆ cycloalkoxy, C₆₋₁₀ arylcarbonyl, C₁₋₃ alkoxy-C₁₋₃ alkoxy, carboxy, C₁₋₃ alkoxycarbonyl, and C₆₋₁₀ aryloxy.

In some embodiments, R¹ is C₁₋₆ alkylcarbonyl, wherein C₁₋₆ alkyl in the C₁₋₆ alkylcarbonyl is optionally substituted with 1, 2, or 3 substituents independently selected from C₁₋₆ alkoxy, C₁₋₆ cycloalkoxy, C₆₋₁₀ arylcarbonyl, C₁₋₃ alkoxy-C₁₋₃ alkoxy, carboxy, C₁₋₃ alkoxycarbonyl, and C₆₋₁₀ aryloxy.

In some embodiments, R¹ is C₁₋₆ alkylcarbonyl, wherein C₁₋₆ alkyl in the C₁₋₆ alkylcarbonyl is optionally substituted with 1, 2, or 3 substituents independently selected from Cy⁴, amino, and C₁₋₃ alkoxy.

In some embodiments, R¹ is C₁₋₆ alkylcarbonyl, wherein C₁₋₆ alkyl in the C₁₋₆ alkylcarbonyl is optionally substituted with Cy⁴ and amino.

In some embodiments, R¹ is C₁₋₆ alkylcarbonyl, wherein C₁₋₆ alkyl in the C₁₋₆ alkylcarbonyl is optionally substituted with Cy⁴ and C₁₋₆ alkoxy.

In some embodiments, R¹ is selected from C₁₋₆ alkoxycarbonyl and C₁₋₆ alkylcarbonyl.

In some embodiments, R¹ is selected from: —C(O)methyl, —C(O)ethyl, —C(O)propyl, —C(O)butyl, —C(O)hexyl, wherein said methyl, ethyl, propyl, butyl, and hexyl are each optionally substituted with 1, 2, or 3 substituents independently selected from: Cy⁴, amino, C₁₋₆ alkoxy, C₁₋₆ cycloalkoxy, C₆₋₁₀ arylcarbonyl, C₁₋₃ alkoxy-C₁₋₃ alkoxy, carboxy, C₁₋₃ alkoxycarbonyl, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R¹ is selected from: —C(O)methyl, —C(O)ethyl, C(O)propyl, —C(O)butyl, —C(O)hexyl, wherein said methyl, ethyl, propyl, butyl, and hexyl are each optionally substituted with 1, 2, or 3 substituents independently selected from: Cy⁴, amino, methoxy, carboxy, (methoxy)carbonyl, phenoxy, —N(methyl)C(O)(t-butoxy), —NHC(O)(t-butoxy), —NHC(O)(benzoxy), —NH(acetyl), methoxy(ethoxy), —NHC(O)(pentyl), and —NHC(O)(isopropyl).

In some embodiments, R¹ is selected from: —C(O)methyl, —C(O)ethyl, —C(O)propyl, —C(O)butyl, —C(O)hexyl, wherein said methyl, ethyl, propyl, butyl, and hexyl are each optionally substituted with 1, 2, or 3 substituents independently selected from: Cy⁴, amino, methoxy, phenoxy, —N(methyl)C(O)(t-butoxy), —NHC(O)(t-butoxy), —NHC(O)(benzoxy), —NH(acetyl), (methoxy)ethoxy, —NHC(O)(pentyl), and —NHC(O)(isopropyl).

In some embodiments, R² is H.

In some embodiments, R² is C₁₋₆alkoxycarbonyl.

In some embodiments, R² is C₁₋₆alkylcarbonyl.

In some embodiments, R³ is selected from: C₁₋₆ alkoxycarbonyl, C₁₋₆ alkylcarbonyl, C(O)NH(R^{a1}), and C(O)Cy⁴, wherein C₁₋₆alkyl in the C₁₋₆alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl,. C₁₋₃ alkoxy-C₁₋₃ alkoxy, carboxy, C₆₋₁₀ aryloxy, —N(C₁₋₃ alkyl) (C₁₋₆ alkoxycarbonyl), —NH(C₁₋₆ alkoxycarbonyl), and —NH(C₁₋₆ alkylcarbonyl), and wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R³ is H.

In some embodiments, R³ is C₁₋₆ alkoxycarbonyl (e.g., ethoxycarbonyl, t-bytoxycarbonyl, isopropoxycarbonyl).

In some embodiments, R³ is C₁₋₆alkylcarbonyl (e.g., methylcarbonyl, ethyl carbonyl, propylcarbonyl, butylcarbonyl, pentylcarbonyl, or hexylcarbonyl).

In some embodiments, R³ is C(O)NH(R^{a1}).

In some embodiments, R³ is C(O)Cy⁴.

In some embodiments, R³ is C₁₋₆ alkylcarbonyl, wherein C₁₋₆ alkyl in the C₁₋₆ alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl,·C₁₋₃ alkoxy-C₁₋₃ alkoxy, carboxy, C₁₋₃alkoxycarbonyl, C₆₋₁₀ aryloxy, —N(C₁₋₃ alkyl)(C₁₋₆alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R³ is C₁₋₆ alkylcarbonyl, wherein C₁₋₆ alkyl in the C₁₋₆ alkylcarbonyl is optionally substituted with Cy⁴.

In some embodiments, R³ is C₁₋₆ alkylcarbonyl, wherein C₁₋₆ alkyl in the C₁₋₆ alkylcarbonyl is optionally substituted with 1, 2, or 3 substituents independently selected from amino, C₁₋₆ alkoxy, C₁₋₆ cycloalkoxy, C₆₋₁₀ arylcarbonyl,·C₁₋₃ alkoxy-C₁₋₃ alkoxy, carboxy, C₁₋₃ alkoxycarbonyl, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R³ is C₁₋₆ alkylcarbonyl, wherein C₁₋₆ alkyl in the C₁₋₆ alkylcarbonyl is optionally substituted with 1, 2, or 3 substituents independently selected from amino, C₁₋₆ alkoxy, C₁₋₆ cycloalkoxy, C₆₋₁₀ arylcarbonyl,·C₁₋₃ alkoxy-C₁₋₃ alkoxy, carboxy, C₁₋₃ alkoxycarbonyl, and C₆₋₁₀ aryloxy.

In some embodiments, R³ is C₁₋₆ alkylcarbonyl, wherein C₁₋₆ alkyl in the C₁₋₆ alkylcarbonyl is optionally substituted with 1, 2, or 3 substituents independently selected from Cy⁴, amino, and C₁₋₆ alkoxy.

In some embodiments, R³ is C₁₋₆ alkylcarbonyl, wherein C₁₋₆ alkyl in the C₁₋₆ alkylcarbonyl is optionally substituted with Cy⁴ and amino.

In some embodiments, R³ is C₁₋₆ alkylcarbonyl, wherein C₁₋₆ alkyl in the C₁₋₆ alkylcarbonyl is optionally substituted with Cy⁴ and C₁₋₆ alkoxy.

In some embodiments, R³ is selected from C₁₋₆ alkoxycarbonyl and C₁₋₆ alkylcarbonyl.

In some embodiments, R³ is selected from: —C(O)methyl, —C(O)ethyl, —C(O)propyl, —C(O)butyl, —C(O)hexyl, wherein said methyl, ethyl, propyl, butyl, and hexyl are each optionally substituted with 1, 2, or 3 substituents independently selected from: Cy⁴, amino, C₁₋₆ alkoxy, C₁₋₆ cycloalkoxy, C₆₋₁₀ arylcarbonyl,·C₁₋₃alkoxy-C₁₋₃alkoxy, carboxy, C₁₋₃alkoxycarbonyl, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R³ is selected from: —C(O)methyl, —C(O)ethyl, —C(O)propyl, —C(O)butyl, —C(O)hexyl, wherein said methyl, ethyl, propyl, butyl, and hexyl are each optionally substituted with 1, 2, or 3 substituents independently selected from: Cy⁴, amino, methoxy, carboxy, (methoxy)carbonyl, phenoxy, —N(methyl)C(O)(t-butoxy), —NHC(O)(t-butoxy), —NHC(O)(benzoxy), —NH(acetyl), methoxy(ethoxy), —NHC(O)(pentyl), and —NHC(O)(isopropyl).

In some embodiments, R³ is selected from: —C(O)methyl, —C(O)ethyl, —C(O)propyl, —C(O)butyl, —C(O)hexyl, wherein said methyl, ethyl, propyl, butyl, and hexyl are each optionally substituted with 1, 2, or 3 substituents independently selected from: Cy⁴, amino, methoxy, phenoxy, —N(methyl)C(O)(t-butoxy), —NHC(O)(t-butoxy), —NHC(O)(benzoxy), —NH(acetyl), (methoxy)ethoxy, —NHC(O)(pentyl), and —NHC(O)(isopropyl).

In some embodiments, R⁴ is H.

In some embodiments, R⁴ is C₁₋₆alkoxycarbonyl.

In some embodiments, R⁴ is C₁₋₆alkylcarbonyl (e.g., methylcarbonyl).

In some embodiments, each of R² and R⁴ is a substituent other than H.

In some embodiments, R² is H, and R⁴ is a substituent other than H.

In some embodiments, R⁴ is H, and R² is a substituent other than H.

In some embodiments, R² and R⁴ are each independently selected from C₁₋₆ alkoxycarbonyl and C₁₋₆ alkylcarbonyl; and R¹ and R³ are each independently selected from C₁₋₆ alkoxycarbonyl and C₁₋₆ alkylcarbonyl.

In some embodiments, R¹, R², R³ and R⁴ are each H.

In some embodiments, R¹ and R³ are each independently selected from H, C₁₋₆ alkoxycarbonyl and C₁₋₆ alkylcarbonyl; and R² and R⁴ are each independently selected from C₁₋₆ alkoxycarbonyl and C₁₋₆ alkylcarbonyl.

In some embodiments, R¹ and R³ are each C₁₋₆ alkoxycarbonyl; and R² and R⁴ are each C₁₋₆ alkoxycarbonyl.

In some embodiments, R¹ and R³ are each C₁₋₆ alkylcarbonyl; and R² and R⁴ are each C₁₋₆ alkylcarbonyl.

In some embodiments, R¹ and R³ are each C₁₋₆ alkoxycarbonyl; and R² and R⁴ are each C₁₋₆ alkylcarbonyl.

In some embodiments, R¹ and R² are each C₁₋₆ alkoxycarbonyl; and R³ and R⁴ are each C₁₋₆ alkylcarbonyl.

In some embodiments, Y is selected from O and C(═O), R¹ is C₁₋₆ alkoxycarbonyl, and R³ is C₁₋₆ alkylcarbonyl.

In some embodiments, R¹ and R² are each H; and R³ and R⁴ are each independently selected from H, C₁₋₆ alkoxycarbonyl, and C₁₋₆ alkylcarbonyl.

In some embodiments, R¹ and R² are each H, R³ is C₁₋₆alkoxycarbonyl and R⁴ is C₁₋₆ alkylcarbonyl. In some aspects of these embodiments, Y is O.

In some embodiments, R¹ and R² are each H; and R³ and R⁴ are each independently selected from H, C₁₋₆ alkoxycarbonyl, C₁₋₆ alkylcarbonyl, and C(O)NH(R^{a1}).

In some embodiments, R¹ and R² are each H, and R³ and R⁴ are each independently selected from C₁₋₆ alkoxycarbonyl, C₁₋₆ alkylcarbonyl, and C(O)NH(R^{a1}).

In some embodiments, R¹ and R² are each H, and R³ and R⁴ are each independently selected from H and C(O)NH(R^{a1}).

In some embodiments, the compound of Formula (I) has Formula (Ia):

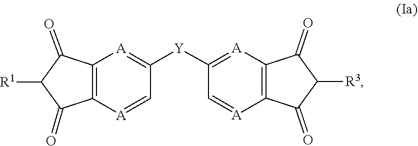

or a pharmaceutically acceptable salt thereof.

In some embodiments, R¹ and R³ are each independently selected from: C₁₋₆ alkoxycarbonyl, C₁₋₆ alkylcarbonyl, C(O)NH(R^{a1}), and C(O)Cy⁴, wherein C₁₋₆ alkyl in the C₁₋₆ alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl, C₁₋₃alkoxy, C₁₋₃alkoxy-C₁₋₃alkoxy, carboxy, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), and wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R¹ and R³ are each independently an C₁₋₆ alkylcarbonyl, wherein C₁₋₆ alkyl in the C₁₋₆ alkylcarbonyl group is optionally substituted with 1, 2, or 3 substituents independently selected from Cy⁴, amino, C₁₋₆ alkoxy, C₁₋₆ cycloalkoxy, C₆₋₁₀ arylcarbonyl, C₁₋₃ alkoxy, C₁₋₃ alkoxy-C₁₋₃ alkoxy, carboxy, C₁₋₃ alkoxycarbonyl, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R¹ and R³ are each independently selected from: halo, CN, C₁₋₆ alkylcarbonyl, C(O)NH(R^{a1}), and C(O)Cy⁴, wherein C₁₋₆ alkyl in the C₁₋₆ alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl, C₁₋₃ alkoxy, C₁₋₃ alkoxy-C₁₋₃ alkoxy, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments, R¹ and R³ are each independently selected from: —C(O)methyl, —C(O)ethyl, —C(O)propyl, —C(O)butyl, —C(O)hexyl, wherein said methyl, ethyl, propyl, butyl, and hexyl are each optionally substituted with 1, 2, or 3 substituents independently selected from: Cy⁴, amino, methoxy, t-butoxy, carboxy, (methoxy)carbonyl, phenoxy, —N(methyl)C(O)(t-butoxy), —NHC(O)(t-butoxy), —NHC(O)(benzoxy), —NH(acetyl), —NHC(O)(pentyl), and —NHC(O)(isopropyl).

In some embodiments, R¹ and R³ are each independently selected from: —C(O)methyl, —C(O)ethyl, —C(O)propyl, —C(O)butyl, —C(O)hexyl, wherein said methyl, ethyl, propyl, butyl, and hexyl are each optionally substituted with 1, 2, or 3 substituents independently selected from: Cy⁴, amino, methoxy, phenoxy, t-butoxy, cyclohexyloxy, —N(methyl)C(O)(t-butoxy), —NHC(O)(t-butoxy), —NHC(O)(benzoxy), —NH(acetyl), (methoxy)ethoxy, —NHC(O)(pentyl), and —NHC(O)(isopropyl).

In some embodiments, R¹ and R³ are each independently selected from C₁₋₆ alkoxycarbonyl, C₁₋₆alkylcarbonyl, and C(O)NH(R^{a1}).

In some embodiments, R¹ and R³ are each independently selected from C₁₋₆ alkoxycarbonyl and C₁₋₆ alkylcarbonyl.

In some embodiments, R¹ and R³ are each independently selected from C(O)NH(R^{a1}) and C(O)Cy⁴.

In some embodiments, R¹ and R³ are each independently selected from: C₁₋₆ alkylcarbonyl and C(O)Cy⁴, wherein C₁₋₆ alkyl in the C₁₋₆ alkylcarbonyl is optionally substituted with Cy⁴.

In some embodiments, R¹ and R³ are each independently selected from: C₁₋₆ alkylcarbonyl and C(O)Cy⁴, wherein C₁₋₆ alkyl in the C₁₋₆ alkylcarbonyl is substituted with Cy⁴.

In some embodiments, R¹ and R³ are each independently an C₁₋₆ alkoxycarbonyl (ethoxycarbonyl, isopropoxycarbobyl, or tert-butoxycarbonyl).

In some embodiments, R¹ and R³ are each independently CN or bromo. In some embodiments, the compound of Formula (Ia) has formula:

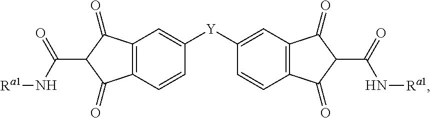

or a pharmaceutically acceptable salt thereof.

In some embodiments, R^{a1} is C₁₋₆ alkyl, optionally substituted with 1, 2, or 3 substituents independently selected from Cy², carboxyl, C₁₋₃ alkoxycarbonyl, C₁₋₃ alkoxy, C₁₋₃ alkoxy-C₁₋₃ alkoxy, and C₁₋₃ haloalkoxy.

In some embodiments, R^{a1} is C₁₋₆ alkyl, optionally substituted with 1 or 2 substituents independently selected from Cy², carboxyl, and C₁₋₃ alkoxycarbonyl.

In some embodiments, R^{a1} is selected from methyl, ethyl, propyl, isopropyl, and sec-butyl, each of which is optionally substituted with 1 or 2 substituents independently selected from Cy², carboxyl, C₁₋₃ alkoxycarbonyl, C₁₋₃ alkoxy, and C₁₋₃ haloalkoxy wherein said C₁₋₃ alkoxy is optionally substituted with C₁₋₃ alkoxy.

In some embodiments, R^{a1} is selected from methyl, ethyl, propyl, isopropyl, and sec-butyl, each of which is optionally substituted with 1 or 2 substituents independently selected from Cy², carboxyl, C₁₃ alkoxycarbonyl.

In some embodiments, R^{a1} is C₁₋₆ alkenyl.

In some embodiments, R^{a1} is Cy¹, optionally substituted with 1 or 2 substituents independently selected from halo, NO₂, C₁₋₆ alkyl, C₁₋₃ alkoxy, C₁₋₆ alkoxycarbonyl, C₁₋₆ alkylcarbonyl, and Cy³.

In some embodiments, R^{a1} is Cy¹, optionally substituted with 1 or 2 substituents independently selected from halo, NO₂, C₁₋₃ alkyl, C₁₋₃ alkoxy and Cy³.

In some embodiments, R^{a1} is Cy¹, optionally substituted with 1 or 2 substituents independently selected from halo, C₁₋₃ alkyl, C₁₋₃ alkoxy and Cy³.

In some embodiments, R^{a1} is Cy¹, optionally substituted with 1 or 2 substituents independently selected from halo and C₁₋₃ alkoxy.

In some embodiments, R^{a1} is selected from phenyl, dihydrobenzodioxinyl, pyridinyl, tetrahydropyranyl, and cyclopropyl, each of which is independently selected from halo, NO₂, C₁₋₃ alkyl, C₁₋₃ alkoxy and Cy³.

In some embodiments, R^{a1} is selected from phenyl, dihydrobenzodioxinyl, pyridinyl, tetrahydropyranyl, cyclobutyl, and cyclopropyl, each of which is independently selected from halo, C₁₋₃ alkyl, C₁₋₃ alkoxy and Cy³.

In some embodiments, R^{a1} is selected from phenyl, dihydrobenzodioxinyl, pyridinyl and cyclopropyl, each of which is independently selected from halo and C₁₋₃ alkoxy.

In some embodiments, R^{a1} is C₁₋₆ alkoxycarbonyl.

In some embodiments, R^{a1} is S(O)₂R^{a2}.

In some embodiments, R^{a2} is phenyl, optionally substituted with C₁₋₃ alkyl.

In some embodiments, R^{a2} is phenyl, optionally substituted with methyl.

In some embodiments, each R^{a1} is independently selected from C₁₋₆ alkyl, C₁₋₆ alkenyl, Cy¹, and C₁₋₆alkoxycarbonyl, wherein said C₁₋₆ alkyl is optionally substituted with 1, 2, or 3 substituents independently selected from Cy², carboxyl, C₁₋₃ alkoxycarbonyl, C₁₋₃ alkoxy, and C₁₋₃ haloalkoxy wherein said C₁₋₃ alkoxy is optionally substituted with C₁₋₃ alkoxy.

In some embodiments, each R^{a1} is independently selected from C₁₋₆ alkyl, C₁₋₆ alkenyl, Cy¹, C₁₋₆alkoxycarbonyl, and S(O)₂R^{a2}, wherein said C₁₋₆ alkyl is optionally substituted with 1, 2, or 3 substituents independently selected from Cy², carboxyl and C₁₃ alkoxycarbonyl.

In some embodiment, each R^{a1} is independently selected from C₁₋₆ alkyl, Cy¹, and C₁₋₆ alkoxycarbonyl, wherein said C₁₋₆ alkyl is optionally substituted with 1, 2, or 3 substituents independently selected from Cy², carboxyl, and C₁₋₃ alkoxycarbonyl.

In some embodiments, each R^{a1} is independently selected from phenyl, ethyl, propyl, (ethoxy)carbonyl, dichloropyridinyl, (benzodiozolyl)ethyl, (furanyl)ethyl, (phenyl)ethyl, cyclopropyl, (fluorophenyl)ethyl, methoxyphenyl, (phenyl)propyl, phenylmethyl, (fluorophenyl)methyl, (ethoxycarbonyl)ethyl, dihydrobenzodioxinyl, (ethoxycarbonyl)methyl, carboxyethyl, allyl, (methylphenyl)sulfonyl, (trifluoromethoxy)ethyl, (methoxy-ethoxy)ethyl, methylphenyl, cyclobutyl, methoxyethyl, tetrahydropyranyl, isopropyl, nitrophenyl, (pyrimidinyl)phenyl, (phenyl)cyclopropyl, (cyclopropyl)methyl, butyl, chlorophenyl, dichlorophenyl, (chlorophenyl)ethyl, (fluorophenyl)ethyl, (methoxy)propyl, (tetrahydrofuranyl)methyl, (dimethoxy)phenyl, iodophenyl, (ethoxycarbonyl)phenyl, (dimethoxyphenyl)ethyl, (butyl)phenyl, acetylphenyl, and (furanyl)methyl.

In some embodiments, each R^{a1} is independently selected from: phenyl, propyl, (ethoxy)carbonyl, dichloropyridinyl, (benzodiozolyl)ethyl, (furanyl)ethyl, (phenyl)ethyl, cyclopropyl, (fluorophenyl)ethyl, methoxyphenyl, (phenyl)propyl, phenylmethyl, (fluorophenyl)ethyl, (ethoxycarbonyl)ethyl, dihydrobenzodioxinyl, (ethoxycarbonyl)methyl, carboxyethyl, allyl, (methylphenyl)sulfonyl, (trifluoromethoxy)ethyl, (methoxy-ethoxy)ethyl, methylphenyl, cyclobutyl, (methoxy)ethyl, tetrahydropyranyl, isopropyl, (cyclopropyl)methyl, butyl, chlorophenyl, dichlorophenyl, (chlorophenyl)ethyl, (methoxy)propyl, (dimethoxy)phenyl, iodophenyl, (ethoxycarbonyl)phenyl, (dimethoxyphenyl)ethyl, (butyl)phenyl, and acetylphenyl.

In some embodiments, each R^{a1} is independently selected from: phenyl, (phenyl)ethyl, cyclopropyl, (fluorophenyl)ethyl, methoxyphenyl, (phenyl)methyl, (methyl)phenyl, (methoxy)ethyl, nitrophenyl, chlorophenyl, (chlorophenyl)ethyl, (methoxy)propyl, (tetrahydrofuranyl)methyl, (dimethoxy)phenyl, iodophenyl, (ethoxycarbonyl)phenyl, and (furanyl)methyl.

In some embodiments, each R^{a1} is independently selected from propyl, isopropyl, sec-butyl, allyl, phenyl, tosyl, ethoxycarbonyl, methoxyphenyl, nitrophenyl, methylphenyl, ethoxyphenyl, dihydrobenzodioxinyl, dichloropyridinyl, benzyl, fluorobenzyl, (pyrimidin-2-yl)phenyl, cyclopropyl, phenylcyclopropyl, phenylethyl, (trifluoromethoxyphenyl)ethyl, (ethoxycarbonyl)methyl, (ethoxycarbonyl)ethyl, phenylpropyl, (fluorophenyl)ethyl, (chlorophenyl)ethyl, (methylphenyl)ethyl, furanylmethyl, benzodioxolylmethyl, benzodioxolylethyl, (2-methoxyethoxy)ethyl, (2,2,2-trifluoroethoxy)ethyl, (carboxyl)ethyl, tetrahydropyranyl, and (carboxyl)methyl.

In some embodiments, each R^{a1} is independently selected from propyl, allyl, phenyl, ethoxycarbonyl, methoxyphenyl, methylphenyl, nitrophenyl, dihydrobenzodioxinyl, dichloropyridinyl, benzyl, fluorobenzyl, cyclopropyl, phenylethyl, (ethoxycarbonyl)methyl, (ethoxycarbonyl)ethyl, phenylpropyl, (fluorophenyl)ethyl, furanylmethyl, benzodioxolylmethyl, (2-methoxyethoxy)ethyl, (2,2,2-trifluoroethoxy)ethyl, tetrahydropyranyl, and (carboxyl)methyl.

In some embodiment, each R^{a1} is independently selected from propyl, isopropyl, sec-butyl, allyl, phenyl, tosyl, ethoxycarbonyl, methoxyphenyl, ethoxyphenyl, dihydrobenzodioxinyl, dichloropyridinyl, benzyl, fluorobenzyl, (pyrimidin-2-yl)phenyl, cyclopropyl, phenylcyclopropyl, phenylethyl, (trifluoromethoxyphenyl)ethyl, (ethoxycarbonyl)methyl, (ethoxycarbonyl)ethyl, phenylpropyl, (fluorophenyl)ethyl, (chlorophenyl)ethyl, (methylphenyl)ethyl, furanylmethyl, benzodioxolylmethyl, benzodioxolylethyl, (carboxyl)ethyl, and (carboxyl)methyl.

In some embodiments, each R^{a1} is independently selected from propyl, phenyl, ethoxycarbonyl, methoxyphenyl, dihydrobenzodioxinyl, dichloropyridinyl, benzyl, fluorobenzyl, cyclopropyl, phenylethyl, (ethoxycarbonyl)methyl, (ethoxycarbonyl)ethyl, phenylpropyl, (fluorophenyl)ethyl, furanylmethyl, benzodioxolylmethyl, and (carboxyl)methyl.

In some embodiments, Cy¹ is C₃₋₁₀ cycloalkyl, optionally substituted with Cy³.

In some embodiments, Cy¹ is cyclopropyl, optionally substituted with Cy³.

In some embodiments, Cy¹ is C₃₋₁₀ cycloalkyl.

In some embodiments, Cy¹ is selected from cyclopropyl, cyclobutyl, and cyclopentyl.

In some embodiments, Cy¹ is 4-10 membered heterocycloalkyl (e.g., tetrahydropyranyl).

In some embodiment, Cy¹ is C₆₋₁₀ aryl, optionally substituted with 1 or 2 substituents independently selected from halo, NO₂, C₁₋₃ alkyl, C₁₋₃ alkoxy and Cy³. In some aspects of these embodiments, the C₆₋₁₀ aryl is phenyl.

In some embodiment, Cy¹ is C₆₋₁₀ aryl, optionally substituted with 1 or 2 substituents independently selected from C₁₋₃ alkyl, C₁₋₃ alkoxy and Cy³. In some aspects of these embodiments, the C₆₋₁₀ aryl is phenyl.

In some embodiments, Cy¹ is C₆₋₁₀ aryl, optionally substituted with C₁₋₃ alkoxy. In some aspects of these embodiments, the C₆₋₁₀ aryl is phenyl.

In some embodiments, Cy¹ is 5-10 membered heteroaryl, optionally substituted with 1 or 2 substituents independently selected from halo, NO₂, C₁₋₃ alkyl, C₁₋₃ alkoxy and Cy³. In some aspects of these embodiments, the 5-10 membered heteroaryl is pyridinyl.

In some embodiments, Cy¹ is 5-10 membered heteroaryl, optionally substituted with 1 or 2 substituents independently selected from halo, C₁₋₃ alkyl, C₁₋₃ alkoxy and Cy³. In some aspects of these embodiments, the 5-10 membered heteroaryl is pyridinyl.

In some embodiments, Cy¹ is 5-10 membered heteroaryl, optionally substituted with 1, 2 or 3 halo. In some aspects of these embodiments, the 5-10 membered heteroaryl is pyridinyl.

In some embodiments, each Cy¹ is independently selected from C₃₋₁₀ cycloalkyl, 4-10 membered heterocycloalkyl, C₆₋₁₀ aryl, and 5-10 membered heteroaryl, each of which is optionally substituted with 1, 2, or 3 substituents independently selected from halo, NO₂, C₁₋₃ alkyl, and C₁₋₃ alkoxy.

In some embodiments, each Cy¹ is independently selected from C₃₋₁₀ cycloalkyl, C₆₋₁₀ aryl, and 5-10 membered heteroaryl, each of which is optionally substituted with 1, 2, or 3 substituents independently selected from halo, C₁₋₃ alkyl, C₁₋₃ alkoxy and Cy³.

In some embodiments, each Cy¹ is independently selected from C₃₋₁₀ cycloalkyl, C₆₋₁₀ aryl, and 5-10 membered heteroaryl, each of which is optionally substituted with 1, 2, or 3 substituents independently selected from halo and C₁₋₃ alkoxy.

In some embodiments, each Cy¹ is independently selected from C₃₋₁₀ cycloalkyl, 4-10 membered heterocycloalkyl, C₆₋₁₀ aryl, and 5-10 membered heteroaryl, each of which is optionally substituted with 1, 2, or 3 substituents independently selected from halo, C₁₋₆ alkyl, C₁₋₃ alkoxy, C₁₋₆ alkoxycarbonyl, C₁₋₆ alkylcarbonyl, and Cy³.

In some embodiments, each Cy¹ is independently selected from phenyl, methoxyphenyl, ethoxyphenyl, dihydrobenzodioxinyl, dichloropyridinyl, cyclopropyl, methylphenyl, nitrophenyl, (pyrimidin-2-yl)phenyl, tetrahydropyranyl, and phenylcyclopropyl.

In some embodiments, each Cy¹ is independently selected from phenyl, methoxyphenyl, dihydrobenzodioxinyl, dichloropyridinyl, cyclopropyl, methylphenyl, tetrahydropyranyl, and nitrophenyl.

In some embodiments, each Cy¹ is independently selected from phenyl, methoxyphenyl, ethoxyphenyl, dihydrobenzodioxinyl, dichloropyridinyl, cyclopropyl, methylphenyl, (pyrimidin-2-yl)phenyl, and phenylcyclopropyl.

In some embodiments, each Cy¹ is independently selected from phenyl, methoxyphenyl, dihydrobenzodioxinyl, dichloropyridinyl and cyclopropyl.

In some embodiments, Cy¹ is independently selected from phenyl, dihydrobenzodioxinyl, pyridinyl, cyclopropyl, and tetrahydropyranyl, each of which is substituted with 1 or 2 substituents independently selected from halo, NO₂, C₁₋₃ alkyl, C₁₋₃ alkoxy and Cy³.

In some embodiments, Cy¹ is independently selected from phenyl, dihydrobenzodioxinyl, pyridinyl, cyclopropyl, and tetrahydropyranyl, each of which is substituted with 1 or 2 substituents independently selected from halo, C₁₋₃ alkyl, C₁₋₃ alkoxy and Cy³.

In some embodiments, Cy¹ is independently selected from phenyl, dihydrobenzodioxinyl, pyridinyl, cyclopropyl, and tetrahydropyranyl, each of which is substituted with 1 or 2 substituents independently selected from halo and C₁₋₃ alkoxy.

In some embodiments, each Cy¹ is independently selected from: dihydrobenzodioxinyl, phenyl, cyclopropyl, pyridinyl, cyclobutyl, cyclopentyl, and tetrahydropyranyl, each of which is optionally substituted with 1, 2, or 3 substituents independently selected from halo, NO₂, C₁₋₆ alkyl, C₁₋₃ alkoxy, C₁₋₆ alkoxycarbonyl, C₁₋₆ alkylcarbonyl, and Cy³.

In some embodiments, each Cy¹ is independently selected from C₃₋₁₀ cycloalkyl and C₆₋₁₀ aryl, each of which is optionally substituted with 1, 2, or 3 substituents independently selected from halo, NO₂, C₁₋₆ alkyl, C₁₋₃ alkoxy, and C₁₋₆ alkoxycarbonyl.

In some embodiments, each Cy¹ is independently selected from: phenyl and cyclopropyl, each of which is optionally substituted with 1, 2, or 3 substituents independently selected from halo, NO₂, C₁₋₆ alkyl, C₁₋₃ alkoxy, C₁₋₆ alkoxycarbonyl, C₁₋₆ alkylcarbonyl, and Cy³.

In some embodiments, each Cy¹ is optionally substituted with 1, 2, or 3 substituents independently selected from chloro, iodo, methoxy, methyl, methoxycarbonyl, ethoxycarbonyl, butyl, acetyl, and Cy³.

In some embodiments, each Cy¹ is optionally substituted with 1, 2, or 3 substituents independently selected from chloro, iodo, NO₂, methoxy, methyl, and ethoxycarbonyl.

In some embodiments, Cy² is C₆₋₁₀ aryl each of which is optionally substituted with 1 or 2 substituents independently selected from halo, C₁₋₃ alkyl, and C₁₋₃ haloalkoxy. In some aspects of these embodiments, the C₆₋₁₀ aryl is phenyl.

In some embodiments, Cy² is C₆₋₁₀ aryl each of which is optionally substituted with 1 or 2 halo.

In some embodiments, Cy² is selected from phenyl and benzodioxolyl each of which is optionally substituted with 1 or 2 halo. In other embodiments, Cy² is phenyl, optionally substituted with 1 or 2 halo.

In some embodiments, Cy² is 5-10 membered heteroaryl, optionally substituted with 1 or 2 substituents independently selected from halo, C₁₋₃ alkyl, and C₁₋₃ haloalkoxy. In some aspects of these embodiments, the 5-10 membered heteroaryl is thiophenyl.

In some embodiments, each Cy² is independently selected from C₆₋₁₀ aryl and 5-10 membered heteroaryl, each of which is optionally substituted with 1 or 2 halo.

In some embodiment, each Cy² is independently selected from phenyl, trifluoromethoxyphenyl, fluorophenyl, chlorophenyl, methylphenyl, furanyl, and benzodioxolyl.

In some embodiments, each Cy² is independently selected from phenyl, fluorophenyl, furanyl, and benzodioxolyl.

In some embodiments, each Cy² is independently selected from C₆₋₁₀ aryl, C₃₋₁₀ cycloalkyl, and 5-10 membered heteroaryl, each of which is optionally substituted with 1, 2, or 3 substituents independently selected from halo and C₁₋₃ alkoxy.

In some embodiments, each Cy² is independently selected from cyclopropyl, phenyl, fluorophenyl, methoxyphenyl, furanyl, and benzodioxolyl, each of which is optionally substituted with 1, 2, or 3 substituents independently selected from halo, C₁₋₃ alkyl, C₁₋₃ alkoxy, and C₁₋₃ haloalkoxy.

In some embodiments, each Cy² is independently selected from: C₆₋₁₀ aryl, 4-10 membered heterocycloalkyl, and 5-10 membered heteroaryl, each of which is optionally substituted with 1, 2, or 3 independently selected from halo.

In some embodiments, each Cy² is independently selected from phenyl, fluorophenyl, chlorophenyl, tetrahydrofuranyl, and furanyl, each of which is optionally substituted with 1, 2, or 3 substituents independently selected from halo, C₁₋₃ alkyl, C₁₋₃ alkoxy, and C₁₋₃ haloalkoxy.

In some embodiments, each Cy³ is C₆₋₁₀ aryl.

In some embodiments, each Cy³ is 5-10 membered heteroaryl.

In some embodiments, each Cy³ is independently selected from phenyl and pyrimidinyl.

In some embodiments, the compound of Formula (I) has a Formula:

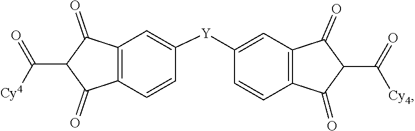

or a pharmaceutically acceptable salt thereof.

In some embodiments, Cy⁴ is C₆₋₁₀ aryl.

In some embodiments, Cy⁴ is C₃₋₁₀ cycloalkyl.

In some embodiments, Cy⁴ is 4-10 membered heterocycloalkyl.

In some embodiments, Cy⁴ is 5-10 membered heteroaryl.

In some embodiments, each Cy⁴ is independently selected from C₃₋₁₀ cycloalkyl and 4-10 membered heterocycloalkyl.

In some embodiments, each Cy⁴ is independently selected from C₆₋₁₀ aryl and C₃₋₁₀ cycloalkyl.

In some embodiments, each Cy⁴ is independently selected from 4-10 membered heterocycloalkyl and 5-10 membered heteroaryl.

In some embodiments, each Cy⁴ is independently selected from pyrrolidinyl, azetidinyl, cyclopropyl, cyclopentyl, cyclohexyl, phenyl, pyridyl, azabicyclohexanyl, tetrahydrofurodioxolyl, dihydropyrrolyl, thiazolidinyl, pyrazolyl, piperidinyl, azaspiroheptanyl, tetrahydro(bisdioxolo)pyranyl, morpholinyl, tetrahydrofuranyl, and tetrazolyl, each of which is optionally substituted with 1, 2, or 3 substituents independently selected from halo, C₁₋₆ alkyl, C₁₋₆ alkoxy, C₁₋₆ alkoxycarbonyl, C₁₋₆ alkylidene, and oxo; wherein C₁₋₆ alkoxy in said C₁₋₆ alkoxycarbonyl is optionally substituted with C₆₋₁₀ aryl.

In some embodiments, each Cy⁴ is independently selected from pyrrolidinyl, azetidinyl, cyclopropyl, cyclopentyl, cyclohexyl, phenyl, pyridyl, azabicyclohexanyl, tetrahydrofurodioxolyl, dihydropyrrolyl, thiazolidinyl, pyrazolyl, piperidinyl, azaspiroheptanyl, tetrahydro(bisdioxolo)pyranyl, morpholinyl, tetrahydrofuranyl, and tetrazolyl, each of which is optionally substituted with 1, 2, or 3 substituents independently selected from: from (t-butoxy)carbonyl, fluoro, (methoxy)carbonyl, methyl, (benzyloxy)carbonyl, methylene, (isopropoxy)carbonyl, methoxy, phenyl, carboxy, (methoxy)carbonyl, and oxo.

In some embodiments, each Cy⁴ is independently selected from pyrrolidinyl, azetidinyl, cyclopropyl, cyclopentyl, cyclohexyl, phenyl, pyridyl, azabicyclohexanyl, dihydropyrrolyl, thiazolidinyl, pyrazolyl, piperidinyl, azaspiroheptanyl, tetrahydro(bisdioxolo)pyranyl, morpholinyl, tetrahydropyranyl, and tetrazolyl, each of which is optionally substituted with 1, 2, or 3 substituents independently selected from halo, C₁₋₆ alkyl, C₁₋₆ alkoxy, C₁₋₆ alkoxycarbonyl, C₁₋₆ alkylidene, and oxo; wherein C₁₋₆ alkoxy in said C₁₋₆ alkoxycarbonyl is optionally substituted with C₆₋₁₀ aryl.

In some embodiments, each Cy⁴ is independently selected from pyrrolidinyl, azetidinyl, cyclopropyl, cyclopentyl, cyclohexyl, phenyl, pyridyl, azabicyclohexanyl, dihydropyrrolyl, thiazolidinyl, pyrazolyl, piperidinyl, azaspiroheptanyl, tetrahydro(bisdioxolo)pyranyl, morpholinyl, tetrahydropyranyl, and tetrazolyl, each of which is optionally substituted with 1, 2, or 3 substituents independently selected from (t-butoxy)carbonyl, fluoro, (methoxy)carbonyl, methyl, (benzyloxy)carbonyl, methylene, (isopropoxy)carbonyl, methoxy, phenyl, carboxy, (methoxy)carbonyl, and oxo.

In some embodiments, each Cy⁴ is independently selected from pyrrolidinyl, azetidinyl, cyclohexyl, azabicyclohexanyl, dihydropyrrolyl, thiazolidinyl, piperidinyl, azaspiroheptanyl, tetrahydro(bisdioxolo)pyranyl, morpholinyl, and tetrahydrofuranyl, each of which is optionally substituted with 1, 2, or 3 substituents independently selected from halo, C₁₋₆ alkyl, C₁₋₆ alkoxy, C₁₋₆ alkoxycarbonyl, C₁₋₆ alkylidene, and oxo; wherein C₁₋₆ alkoxy in said C₁₋₆ alkoxycarbonyl is optionally substituted with C₆₋₁₀ aryl.

In some embodiments, each Cy⁴ is independently selected from pyrrolidinyl, azetidinyl, cyclohexyl, azabicyclohexanyl, dihydropyrrolyl, thiazolidinyl, piperidinyl, azaspiroheptanyl, tetrahydro(bisdioxolo)pyranyl, morpholinyl, and tetrahydrofuranyl, each of which is optionally substituted with 1, 2, or 3 substituents independently selected from (t-butoxy)carbonyl, fluoro, (methoxy)carbonyl, methyl, (benzyloxy)carbonyl, methylene, (isopropoxy)carbonyl, methoxy, phenyl, carboxy, (methoxy)carbonyl, and oxo.

In some embodiments, each Cy⁴ is independently selected from pyrrolidinyl, azetidinyl, cyclohexyl, tetrahydrofurodioxolyl, thiazolidinyl, morpholinyl, and tetrahydrofuranyl, each of which is optionally substituted with 1, 2, or 3 substituents independently selected from halo, C₁₋₆ alkyl, C₁₋₆ alkoxy, C₁₋₆ alkoxycarbonyl, C₁₋₆ alkylidene, and oxo; wherein C₁₋₆ alkoxy in said C₁₋₆ alkoxycarbonyl is optionally substituted with C₆₋₁₀ aryl.

In some embodiments, each Cy⁴ is independently selected from pyrrolidinyl, azetidinyl, cyclohexyl, tetrahydrofurodioxolyl, thiazolidinyl, morpholinyl, and tetrahydrofuranyl, each of which is optionally substituted with 1, 2, or 3 substituents independently selected from (t-butoxy)carbonyl, methyl, (benzyloxy)carbonyl, methoxy, phenyl, carboxy, (methoxy)carbonyl, and oxo.

In some embodiments:
Y is selected from O, C(═O), S(═O)₂, C(R⁵)(R⁶), and a bond between the indane-1,3-dione rings of the compound of Formula (I);
R¹ and R³ are each independently selected from H, C₁₋₆ alkoxycarbonyl, C₁₋₆ alkylcarbonyl, and C(O)NH(R^{a1});
R² and R⁴ are each independently selected from H, C₁₋₆ alkoxycarbonyl, and C₁₋₆ alkylcarbonyl;
R⁵ and R⁶ are each C₁₋₃ haloalkyl;
each R^{a1} is independently selected from C₁₋₆ alkyl, C₁₋₆ alkenyl, Cy¹, C₁₋₆ alkoxycarbonyl, and S(O)₂R^{a2}, wherein said C₁₋₆ alkyl is optionally substituted with 1, 2, or 3 substituents independently selected from Cy², carboxyl, C₁₋₃ alkoxycarbonyl;
each Cy¹ is independently selected from C₃₋₁₀ cycloalkyl, C₆₋₁₀ aryl, and 5-10 membered heteroaryl, each of which is optionally substituted with 1, 2, or 3 substituents independently selected from halo, C₁₋₃ alkyl, C₁₋₃ alkoxy and Cy³;
each Cy² is independently selected from C₆₋₁₀ aryl and 5-10 membered heteroaryl, each of which is optionally substituted with 1, 2, or 3 substituents independently selected from halo, C₁₋₃ alkyl, and C₁₋₃ haloalkoxy;
each Cy³ is independently selected from C₆₋₁₀ aryl and 5-10 membered heteroaryl; and
each R^{a2} is C₆₋₁₀ aryl, optionally substituted with C₁₋₃ alkyl.

In some embodiments:
Y is selected from O, C(═O), S(═O)₂, C(R⁵)(R⁶), and a bond between the indane-1,3-dione rings of the compound of Formula (I);
R¹ and R³ are each independently selected from H, C₁₋₆ alkoxycarbonyl, C₁₋₆ alkylcarbonyl, and C(O)NH(R^{a1});
R² and R⁴ are each independently selected from H, C₁₋₆ alkoxycarbonyl, and C₁₋₆ alkylcarbonyl;
R⁵ and R⁶ are each C₁₋₃ haloalkyl;
each R^{a1} is independently selected from C₁₋₆ alkyl, Cy¹, and C₁₋₆ alkoxycarbonyl, wherein said C₁₋₆ alkyl is optionally substituted with 1, 2, or 3 substituents independently selected from Cy², carboxyl, C₁₋₃ alkoxycarbonyl;
each Cy¹ is independently selected from C₃₋₁₀ cycloalkyl, C₆₋₁₀ aryl, and 5-10 membered heteroaryl, each of which is optionally substituted with 1, 2, or 3 substituents independently selected from halo, and C₁₋₃ alkoxy; and
each Cy² is independently selected from C₆₋₁₀ aryl and 5-10 membered heteroaryl, each of which is optionally substituted with 1, 2, or 3 halo.

In some embodiments:
Y is selected from C(═O) and S(═O)₂;
R² and R⁴ are each H; and
R¹ and R³ are each C(O)NH(R^{a1}).

In some embodiments:
Y is a bond between the between the indane-1,3-dione rings of the compound of Formula (I); and
R¹ and R³ are each independently selected from C₁₋₆ alkoxycarbonyl and C₁₋₆ alkylcarbonyl.

In some aspects of these embodiments, R¹ and R³ are not both C₁₋₆ alkylcarbonyl.

In some embodiments:
Y is selected from C(═O), S(═O)₂, and C(R⁵)(R⁶); and
R¹ and R³ are each independently selected from H, C₁₋₆ alkoxycarbonyl and C₁₋₆ alkylcarbonyl.

In some embodiments:
Y is O; and
R¹ and R³ are each independently selected from H and C₁₋₆ alkylcarbonyl.

In some aspects of these embodiments, R¹ and R³ are not both H.

In some embodiments:
Y is C(═O); and
each R^{a1} is independently selected from C₁₋₆ alkyl, Cy¹ and C₁₋₆ alkoxycarbonyl, wherein said C₁₋₆ alkyl is optionally substituted with 1 or 2 substituents independently selected from Cy², carboxyl and C₁₋₃ alkoxycarbonyl.

In some embodiments:
Y is S(═O)₂; and
each R^{a1} is independently selected from C₁₋₆ alkyl and Cy¹, wherein said C₁₋₆ alkyl is optionally substituted with Cy².

In some embodiments, the compound of Formula (I) has a Formula (Ia):

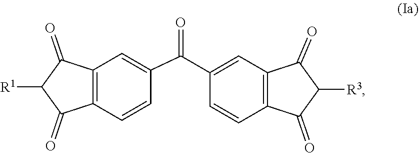

or a pharmaceutically acceptable salt thereof.

In some embodiments of Formula (Ia), R¹ and R³ are each independently selected from: halo, CN, C₁₋₆ alkoxycarbonyl, C₁₋₆ alkylcarbonyl, C(O)NH(R^{a1}), and C(O)Cy⁴, wherein C₁₋₆ alkyl in the C₁₋₆ alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl, carboxy, C₁₋₃ alkoxycarbonyl, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), and wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo. In some embodiments, R¹ and R³ are each independently CN or bromo.

In some embodiments of Formula (Ia), R¹ and R³ are each independently C(O)NH(R^{a1}).

In some embodiments of Formula (Ia), Cy¹ is independently selected from C₃₋₁₀ cycloalkyl, 4-10 membered heterocycloalkyl, C₆₋₁₀ aryl, and 5-10 membered heteroaryl, each of which is optionally substituted with 1, 2, or 3 substituents independently selected from halo, C₁₋₆ alkyl, C₁₋₃ alkoxy, C₁₋₆ alkoxycarbonyl, C₁₋₆ alkylcarbonyl, and Cy³.

In some embodiments of Formula (Ia), Cy¹ is selected from: dihydrobenzodioxinyl, phenyl, cyclopropyl, pyridinyl, cyclobutyl, cyclopentyl, and tetrahydropyranyl.

In some embodiments of Formula (Ia), each Cy¹ is optionally substituted with 1, 2, or 3 substituents independently selected from chloro, iodo, methoxy, methyl, ethoxycarbonyl, butyl, acetyl, and Cy³.

In some embodiments of Formula (Ia), each Cy² is independently selected from C₆₋₁₀ aryl, C₃₋₁₀ cycloalkyl, and 5-10 membered heteroaryl, each of which is optionally substituted with 1, 2, or 3 substituents independently selected from halo and C₁₋₃ alkoxy.

In some embodiments of Formula (Ia), each Cy² is independently selected from cyclopropyl, phenyl, fluorophenyl, methoxyphenyl, furanyl, and benzodioxolyl.

In some embodiments of Formula (Ia), each Cy³ is independently selected from phenyl and pyrimidinyl.

In some embodiments of Formula (Ia), each R^{a1} is independently selected from: phenyl, propyl, (ethoxy)carbonyl, dichloropyridinyl, (benzodiozolyl)ethyl, (furanyl)ethyl, (phenyl)ethyl, cyclopropyl, (fluorophenyl)ethyl, methoxyphenyl, (phenyl)propyl, phenylmethyl, (fluorophenyl)ethyl, (ethoxycarbonyl)ethyl, dihydrobenzodioxinyl, (ethoxycarbonyl)methyl, carboxyethyl, allyl, (methylphenyl)sulfonyl, (trifluoromethoxy)ethyl, (methoxy-ethoxy)ethyl, methylphenyl, cyclobutyl, (methoxy)ethyl, tetrahydropyranyl, isopropyl, (cyclopropyl)methyl, butyl, chlorophenyl, dichlorophenyl, (chlorophenyl)ethyl, (methoxy)propyl, (dimethoxy)phenyl, iodophenyl, (ethoxycarbonyl)phenyl, (dimethoxyphenyl)ethyl, (butyl)phenyl, and acetylphenyl.

In some embodiments of Formula (Ia), R¹ and R³ are each independently C₁₋₆ alkoxycarbonyl.

In some embodiments of Formula (Ia), R¹ and R³ are each independently an C₁₋₆ alkylcarbonyl, wherein C₁₋₆ alkyl in the C₁₋₆ alkylcarbonyl group is optionally substituted with 1, 2, or 3 substituents independently selected from Cy⁴, amino, C₁₋₆ alkoxy, C₁₋₆ cycloalkoxy, C₆₋₁₀ arylcarbonyl, carboxy, C₁₋₃ alkoxycarbonyl, C₆₋₁₀ aryloxy, —N(C₁₋₃ alkyl)(C₁₋₆ alkoxycarbonyl), —NH(C₁₋₆alkoxycarbonyl), and —NH(C₁₋₆alkylcarbonyl), wherein C₁₋₆alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments of Formula (Ia), R¹ and R³ are each independently selected from: —C(O)methyl, —C(O)ethyl, —C(O)propyl, —C(O)butyl, —C(O)hexyl, wherein said methyl, ethyl, propyl, butyl, and hexyl are each optionally substituted with 1, 2, or 3 substituents independently selected from: Cy⁴, amino, methoxy, carboxy, (methoxy)carbonyl, phenoxy, —N(methyl)C(O)(t-butoxy), —NHC(O)(t-butoxy), —NHC(O)(benzoxy), —NH(acetyl), —NHC(O)(pentyl), and —NHC(O)(isopropyl).

In some embodiments of Formula (Ia), R¹ and R³ are each independently C(O)Cy⁴. In some aspects of these embodiments, each Cy⁴ is independently selected from C₃₋₁₀ cycloalkyl and 4-10 membered heterocycloalkyl.

In some embodiments of Formula (Ia), each Cy⁴ is independently selected from pyrrolidinyl, azetidinyl, cyclopropyl, cyclopentyl, cyclohexyl, phenyl, azabicyclohexanyl, dihydropyrrolyl, thiazolidinyl, pyrazolyl, piperidinyl, azaspiroheptanyl, tetrahydro(bisdioxolo)pyranyl, tetrahydropyranyl, and tetrazolyl.

In some embodiments of Formula (Ia), each Cy⁴ is independently selected from pyrrolidinyl, azetidinyl, cyclohexyl, azabicyclohexanyl, dihydropyrrolyl, thiazolidinyl, piperidinyl, azaspiroheptanyl, tetrahydro(bisdioxolo)pyranyl, and tetrahydrofuranyl.

In some embodiments of Formula (Ia), each Cy⁴ is optionally substituted with 1, 2, or 3 substituents independently selected from (t-butoxy)carbonyl, fluoro, (methoxy)carbonyl, methyl, (benzyloxy)carbonyl, methylene, (isopropoxy)carbonyl, methoxy, and oxo.

In some embodiments, R¹ and R³ are each independently CN or bromo.

In some embodiments, the compound of Formula (I) has a Formula (Ib):

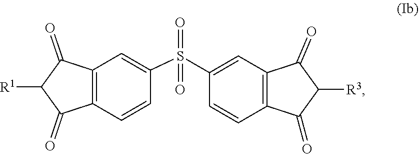

or a pharmaceutically acceptable salt thereof.

In some embodiments of Formula (Ib), R¹ and R³ are each independently selected from: C₁₋₆alkylcarbonyl, C(O)NH(R^{a1}), and C(O)Cy⁴, wherein C₁₋₆alkyl in the C₁₋₆alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl, C₁₋₃ alkoxy-C₁₋₃ alkoxy, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments of Formula (Ib), R¹ and R³ are each independently C(O)NH(R^{a1}).

In some embodiments of Formula (Ib), each Cy¹ is independently selected from C₃₋₁₀ cycloalkyl and C₆₋₁₀ aryl, each of which is optionally substituted with 1, 2, or 3 substituents independently selected from halo, NO₂, C₁₋₆ alkyl, C₁₋₃ alkoxy, and C₁₋₆ alkoxycarbonyl.

In some embodiments of Formula (Ib), Cy¹ is selected from: phenyl and cyclopropyl.

In some embodiments of Formula (Ib), each Cy¹ is optionally substituted with 1, 2, or 3 substituents independently selected from chloro, iodo, NO₂, methoxy, methyl, and ethoxycarbonyl.

In some embodiments of Formula (Ib), each Cy² is independently selected from: C₆₋₁₀ aryl, 4-10 membered heterocycloalkyl, and 5-10 membered heteroaryl, each of which is optionally substituted with 1, 2, or 3 independently selected from halo.

In some embodiments of Formula (Ib), each Cy² is independently selected from phenyl, fluorophenyl, chlorophenyl, tetrahydrofuranyl, and furanyl.

In some embodiments of Formula (Ib), each R^{a1} is independently selected from: phenyl, (phenyl)ethyl, cyclopropyl, (fluorophenyl)ethyl, methoxyphenyl, (phenyl)methyl, (methyl)phenyl, (methoxy)ethyl, nitrophenyl, chlorophenyl, (chlorophenyl)ethyl, (methoxy)propyl, (tetrahydrofuranyl)methyl, (dimethoxy)phenyl, iodophenyl, (ethoxycarbonyl)phenyl, and (furanyl)methyl.

In some embodiments of Formula (Ib), R¹ and R³ are each independently an C₁₋₆ alkylcarbonyl, wherein C₁₋₆ alkyl in the C₁₋₆ alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl, C₁₋₃ alkoxy-C₁₋₃ alkoxy, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl and halo.

In some embodiments of Formula (Ib), R¹ and R³ are each independently selected from: —C(O)methyl, —C(O)ethyl, —C(O)propyl, —C(O)butyl, —C(O)hexyl, wherein said methyl, ethyl, propyl, butyl, and hexyl are each optionally substituted with 1, 2, or 3 substituents independently selected from: Cy⁴, amino, methoxy, phenoxy, —N(methyl)C(O)(t-butoxy), —NHC(O)(t-butoxy), —NHC(O)(benzoxy), —NH(acetyl), (methoxy)ethoxy, —NHC(O)(pentyl), and —NHC(O)(isopropyl).

In some embodiments of Formula (Ib), R¹ and R³ are each independently C(O)Cy⁴. In some aspects of these embodiments, each Cy⁴ is independently selected from C₃₋₁₀ cycloalkyl and 4-10 membered heterocycloalkyl.

In some embodiments of Formula (Ib), each Cy⁴ is independently selected from pyrrolidinyl, azetidinyl, cyclohexyl, tetrahydrofurodioxolyl, thiazolidinyl, and tetrahydrofuranyl.

In some embodiments of Formula (Ib), each Cy⁴ is optionally substituted with 1, 2, or 3 substituents independently selected from (t-butoxy)carbonyl, methyl, (benzyloxy)carbonyl, methoxy, and oxo.

In some embodiments, the compound of Formula (I) has any one of the following formulae:

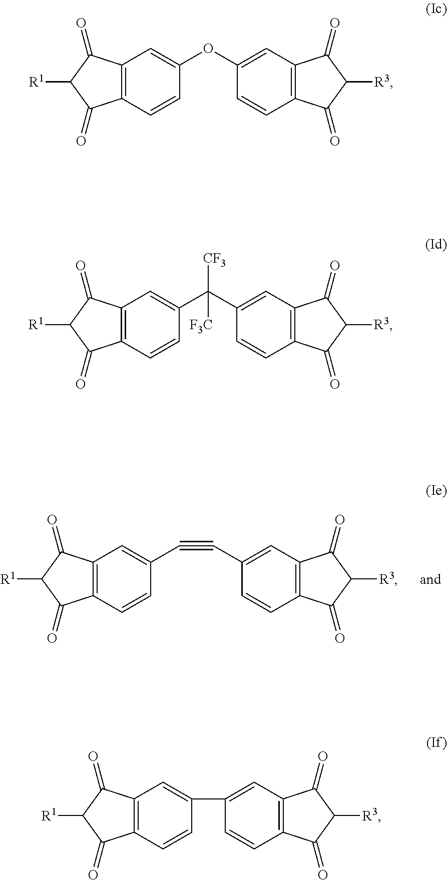

or a pharmaceutically acceptable salt thereof.

In some embodiments, the compound of Formula (I) is not:

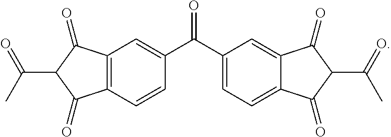 [SMILES: CC(=O)C1C(=O)c2ccc(C(=O)c3ccc4c(c3)C(=O)C(C(C)=O)C4=O)cc2C1=O]

In some embodiments, the compound of Formula (I) is not a compound selected from:

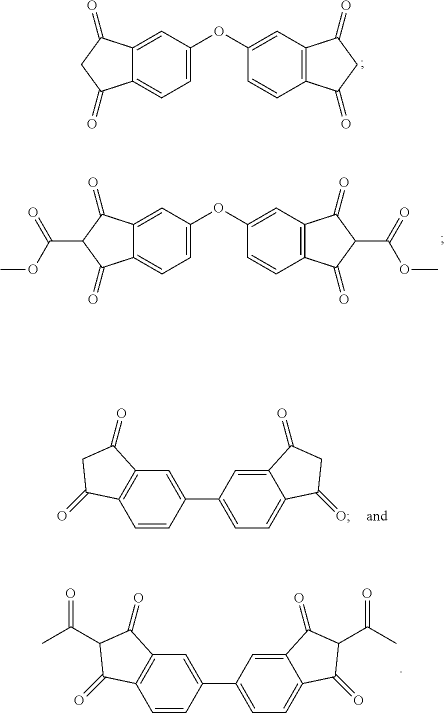

In some embodiments, the compound of Formula (I) is a compound selected from:

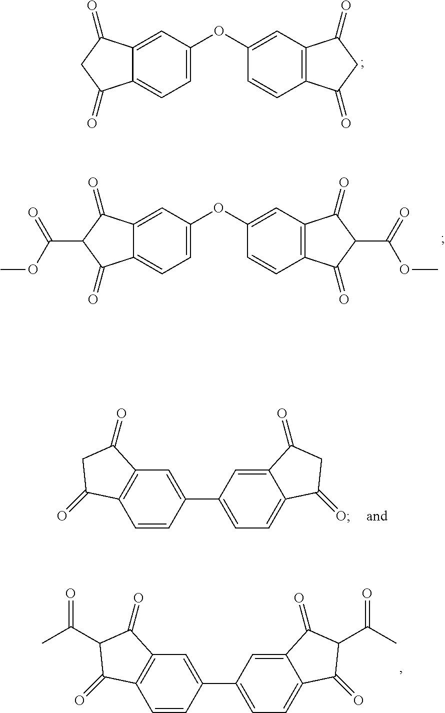

or a pharmaceutically acceptable salt thereof.

In some embodiments, the compound of Formula (I) is:

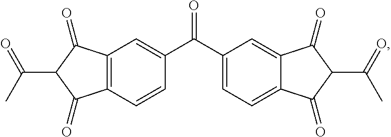 [SMILES: CC(=O)C1C(=O)c2ccc(C(=O)c3ccc4c(c3)C(=O)C(C(C)=O)C4=O)cc2C1=O]

or a pharmaceutically acceptable salt thereof.

In some embodiments, the compound of Formula (I) is a compound selected from:

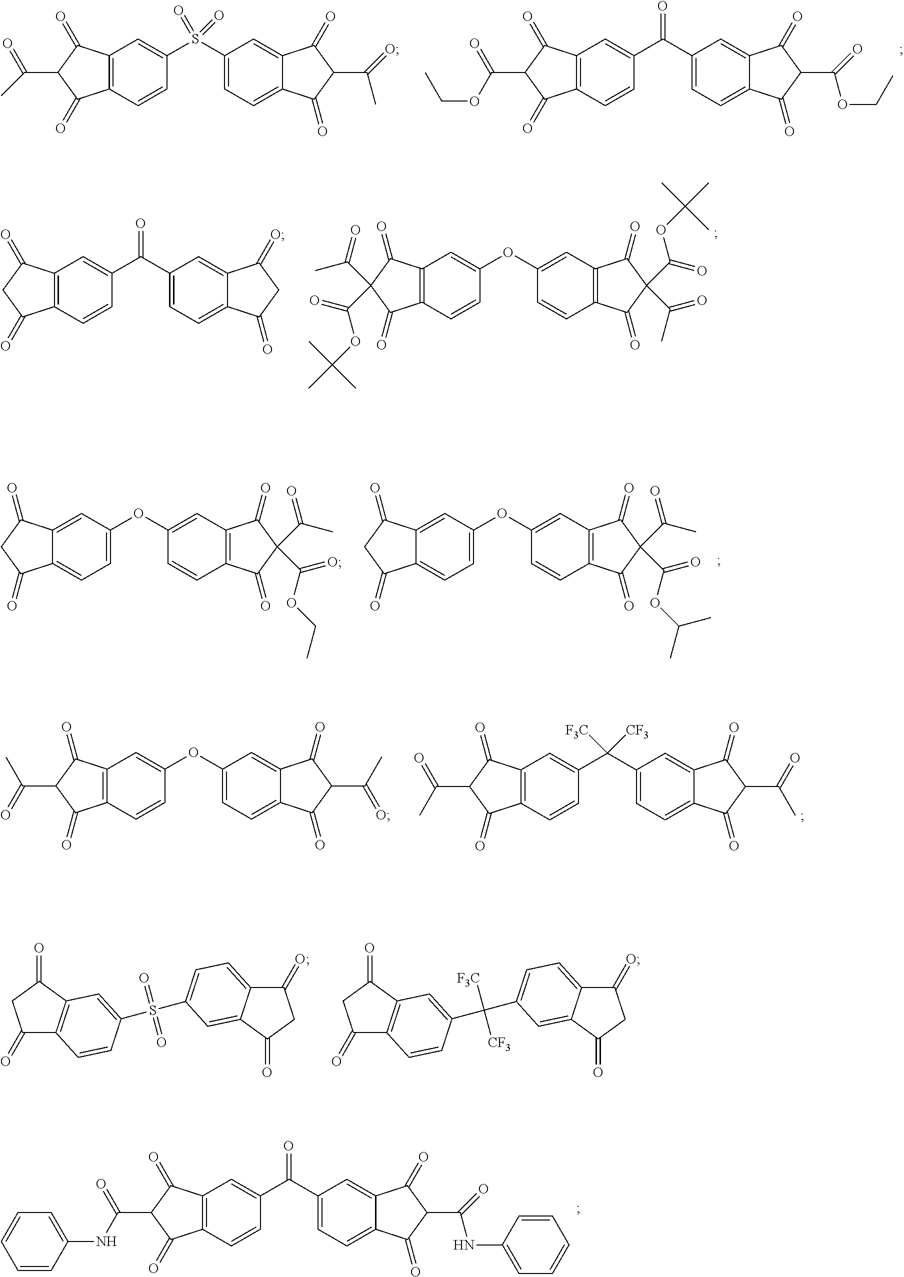

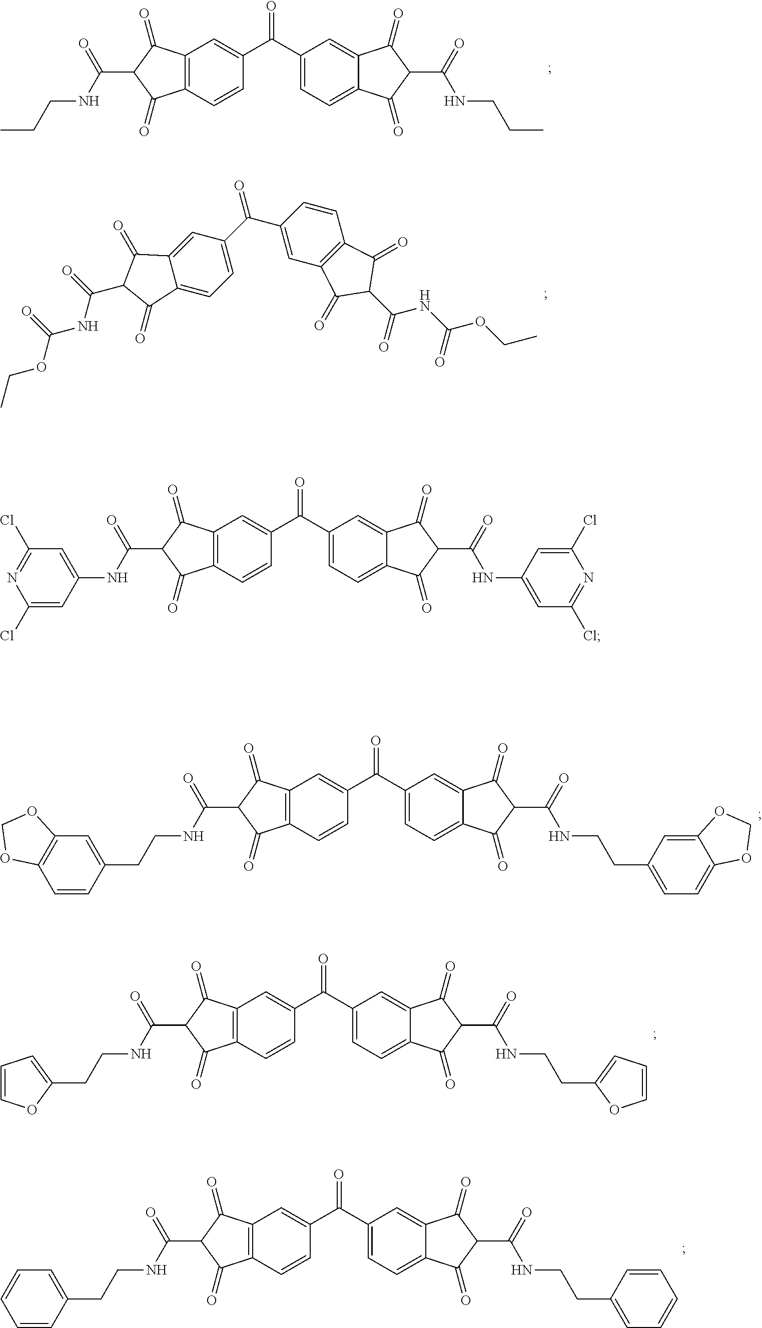

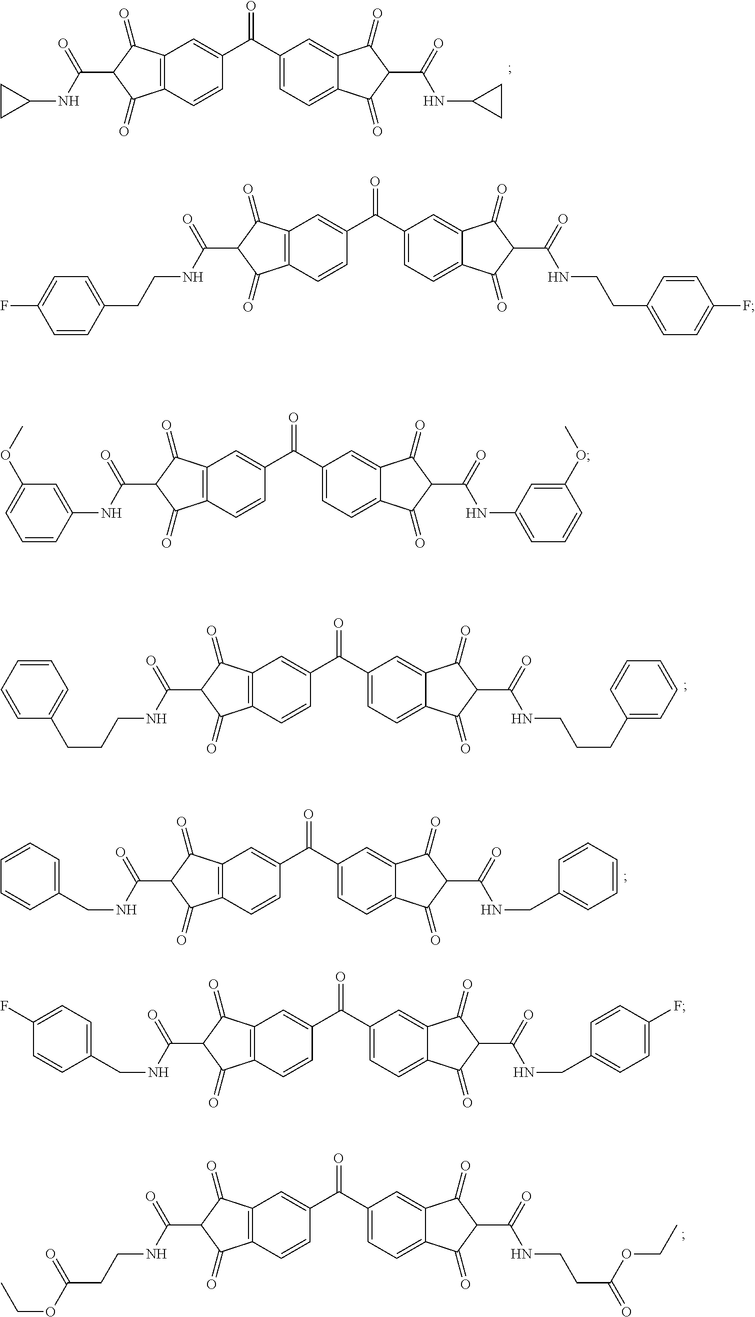

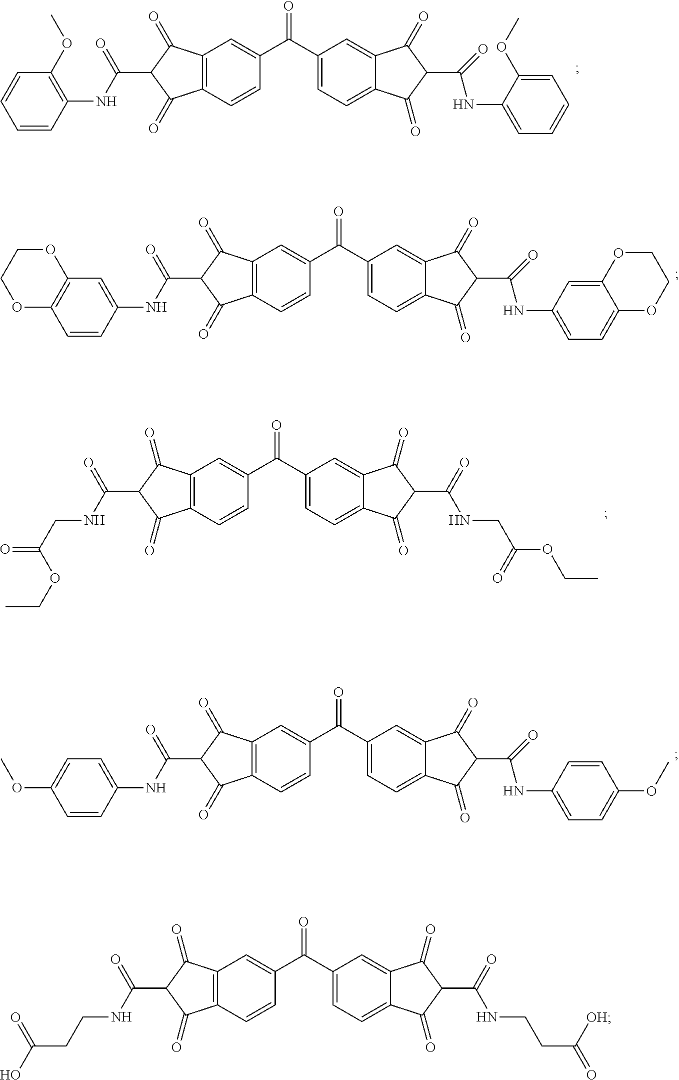

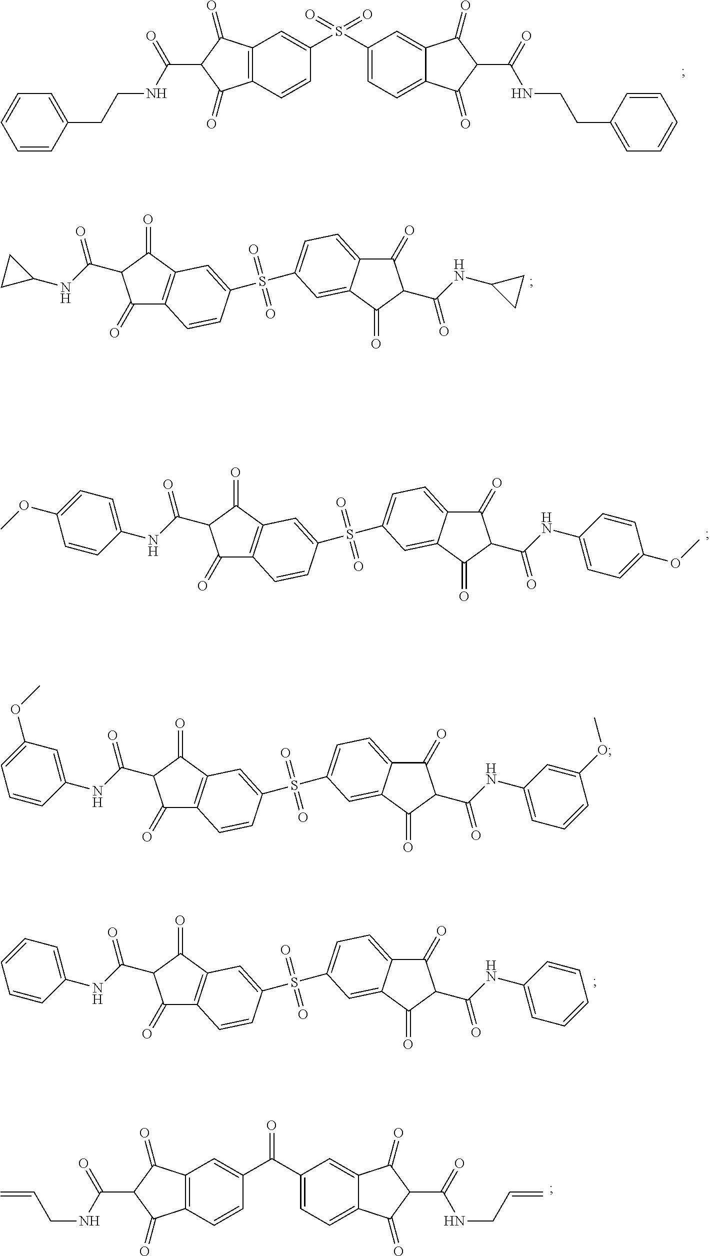

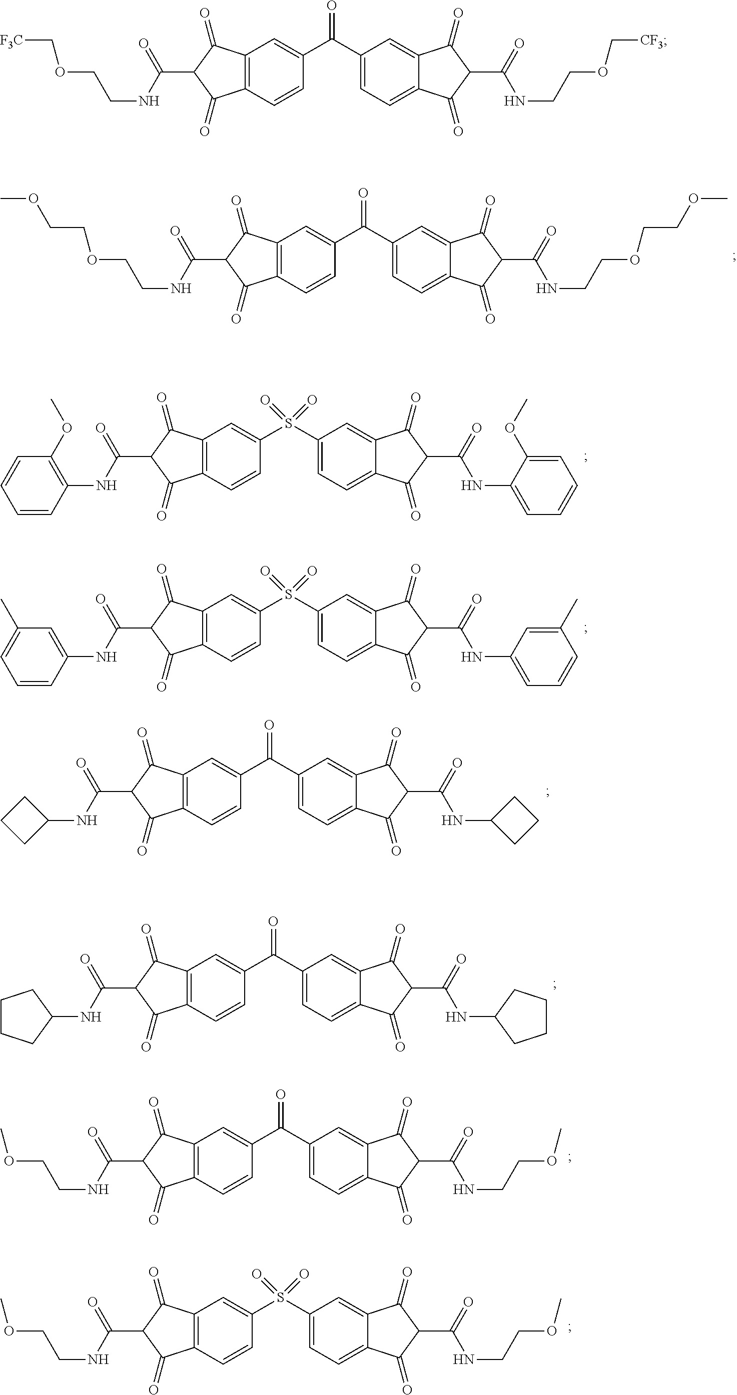

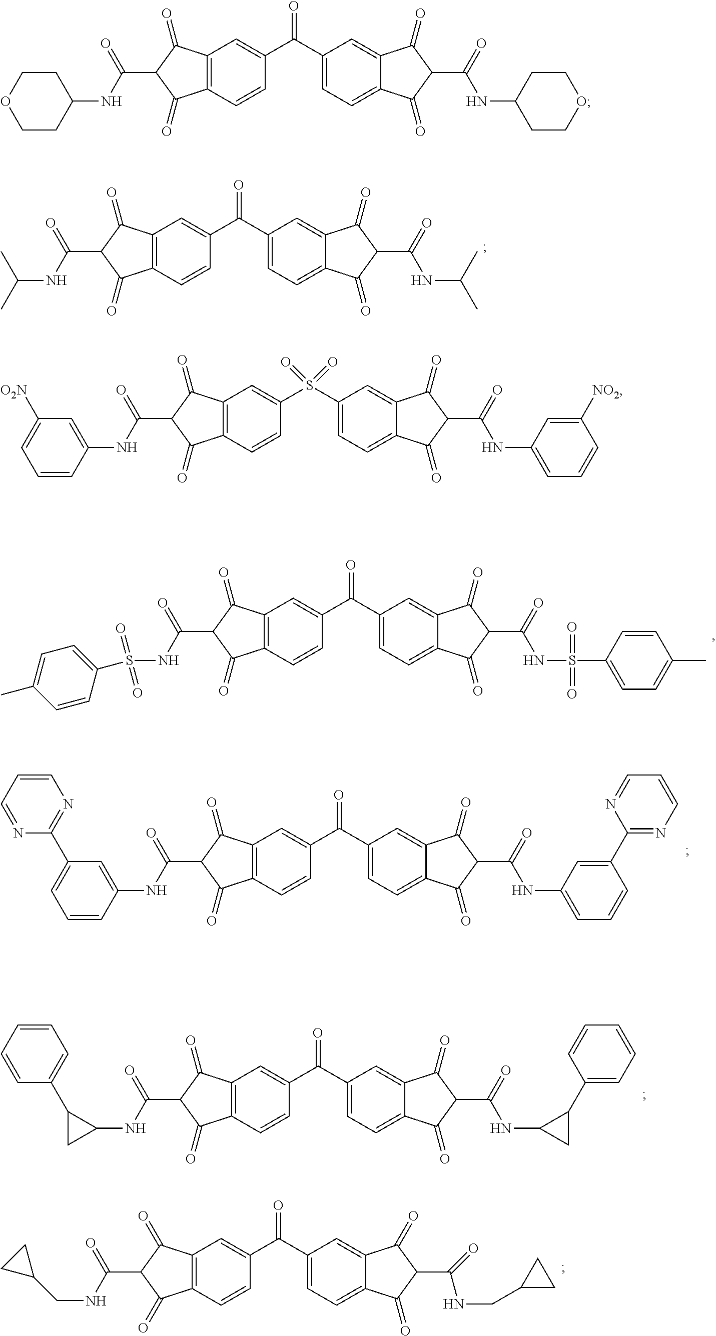

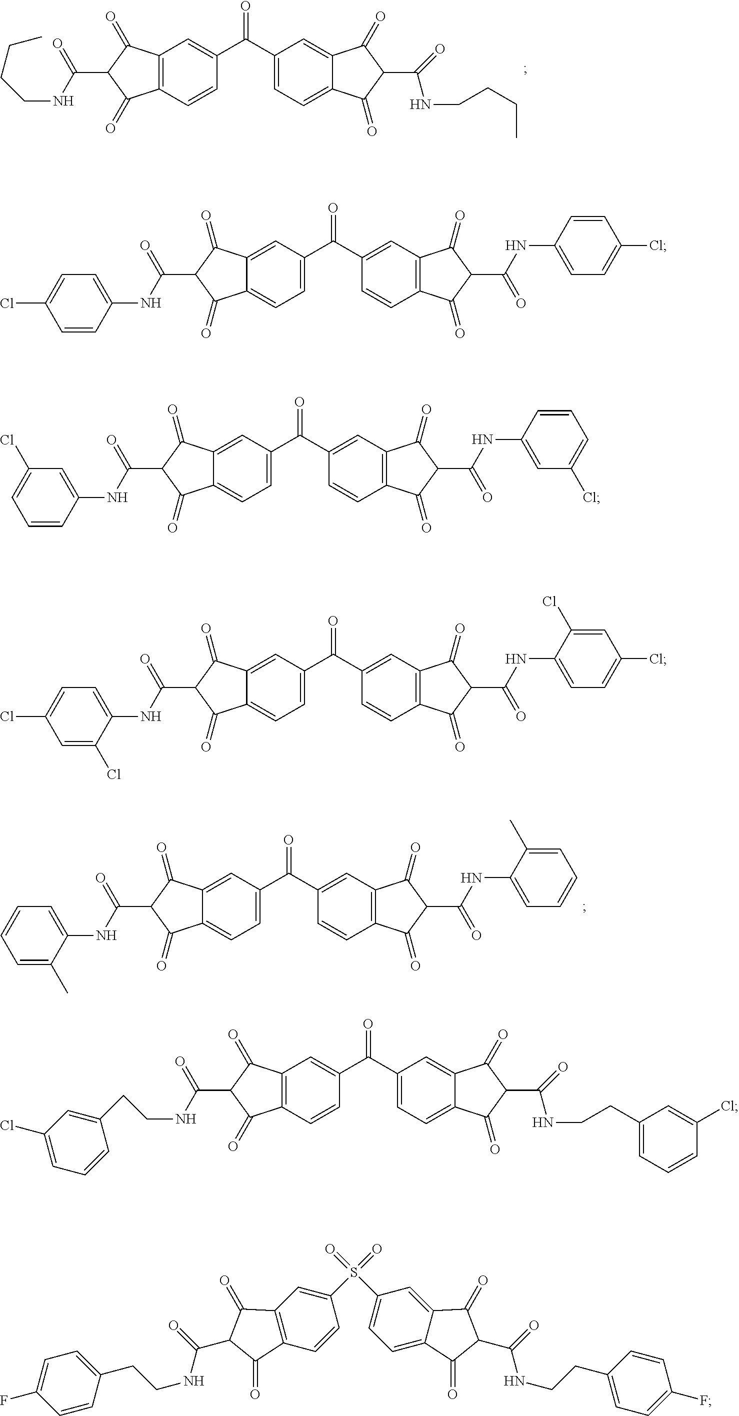

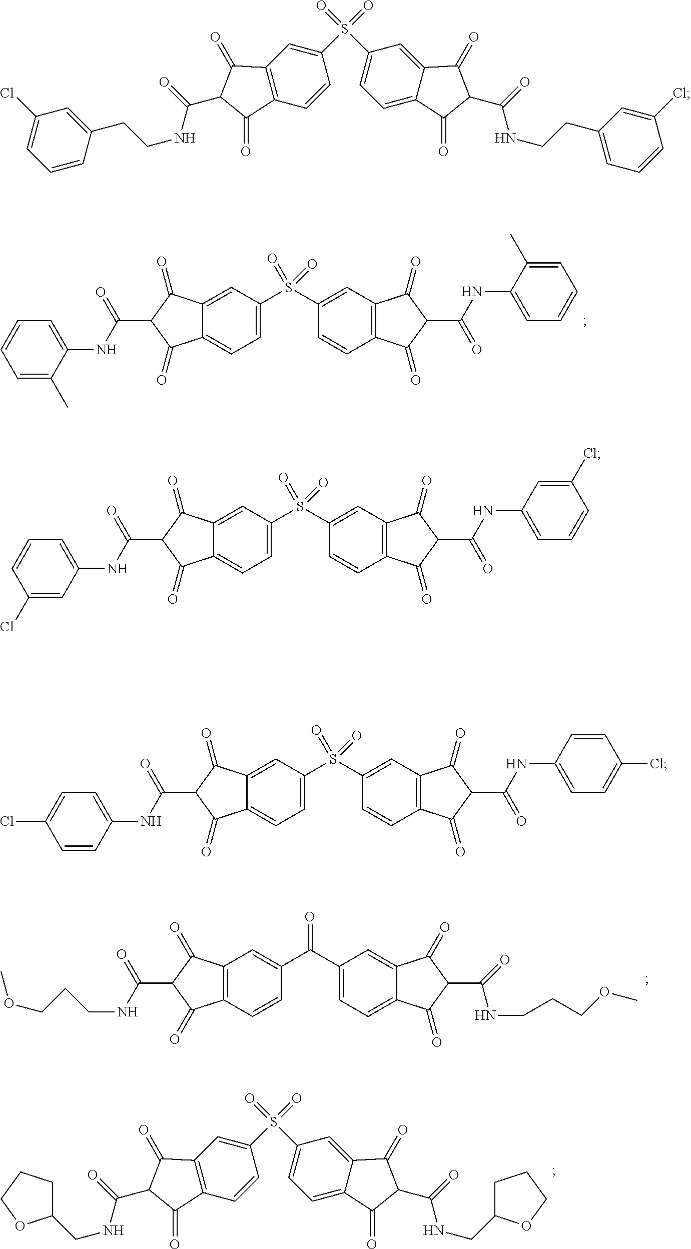

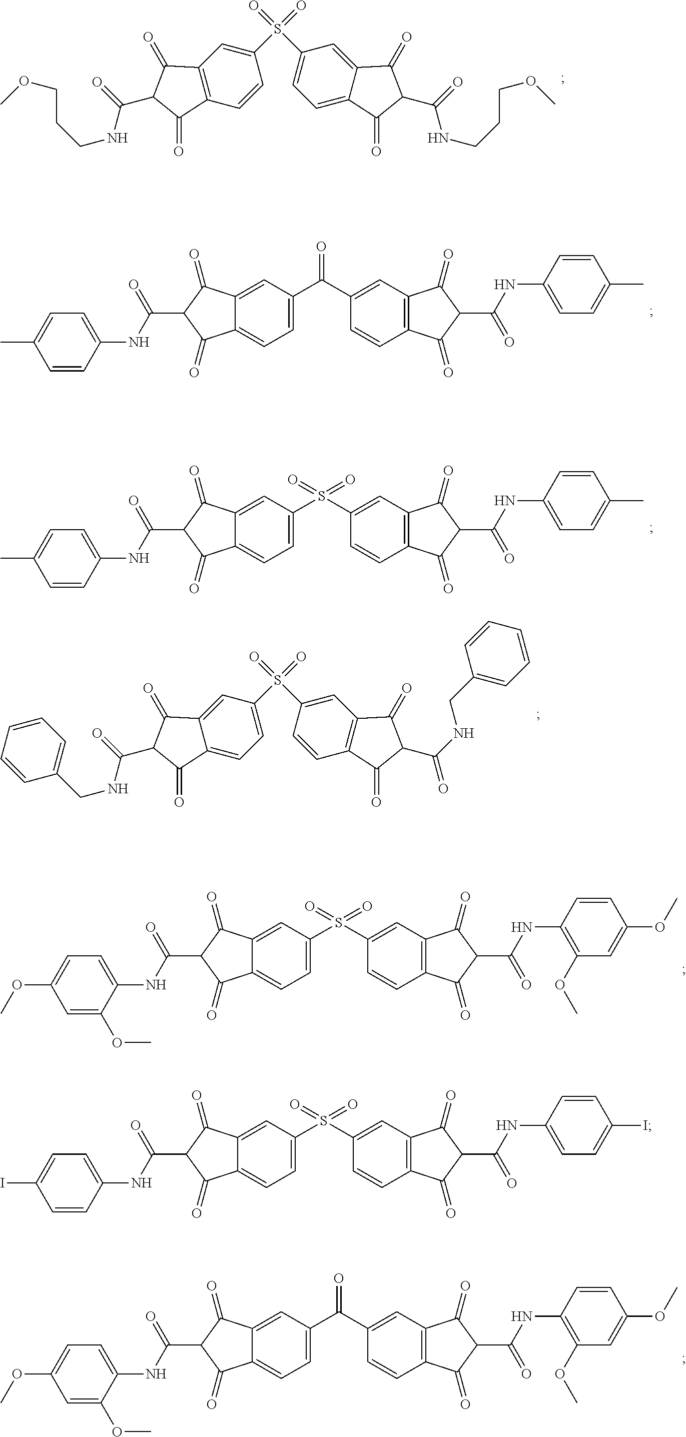

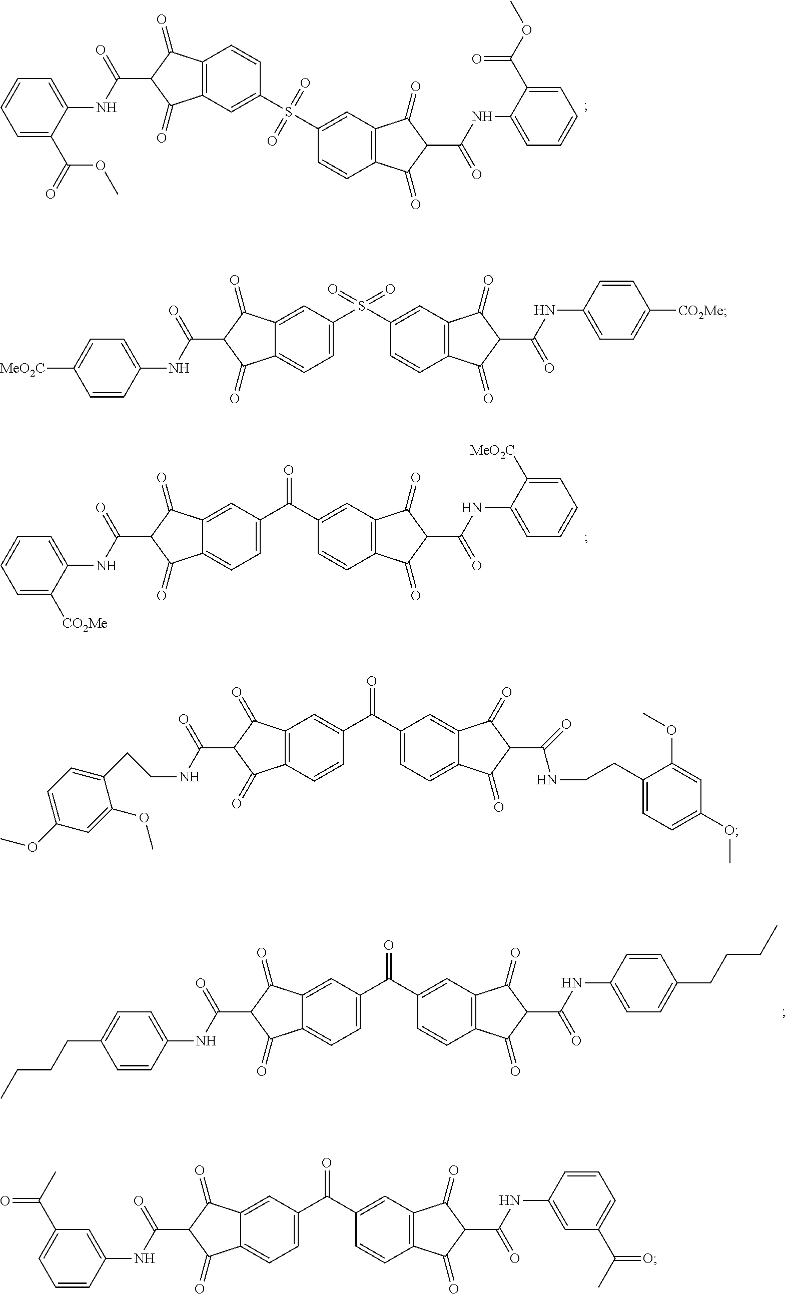

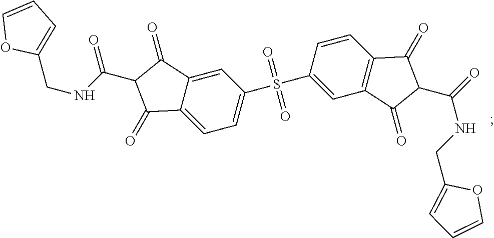 [SMILES: O=C(NCc1ccco1)C1C(=O)c2ccc(S(=O)(=O)c3ccc4c(c3)C(=O)C(C(=O)NCc3ccco3)C4=O)cc2C1=O]

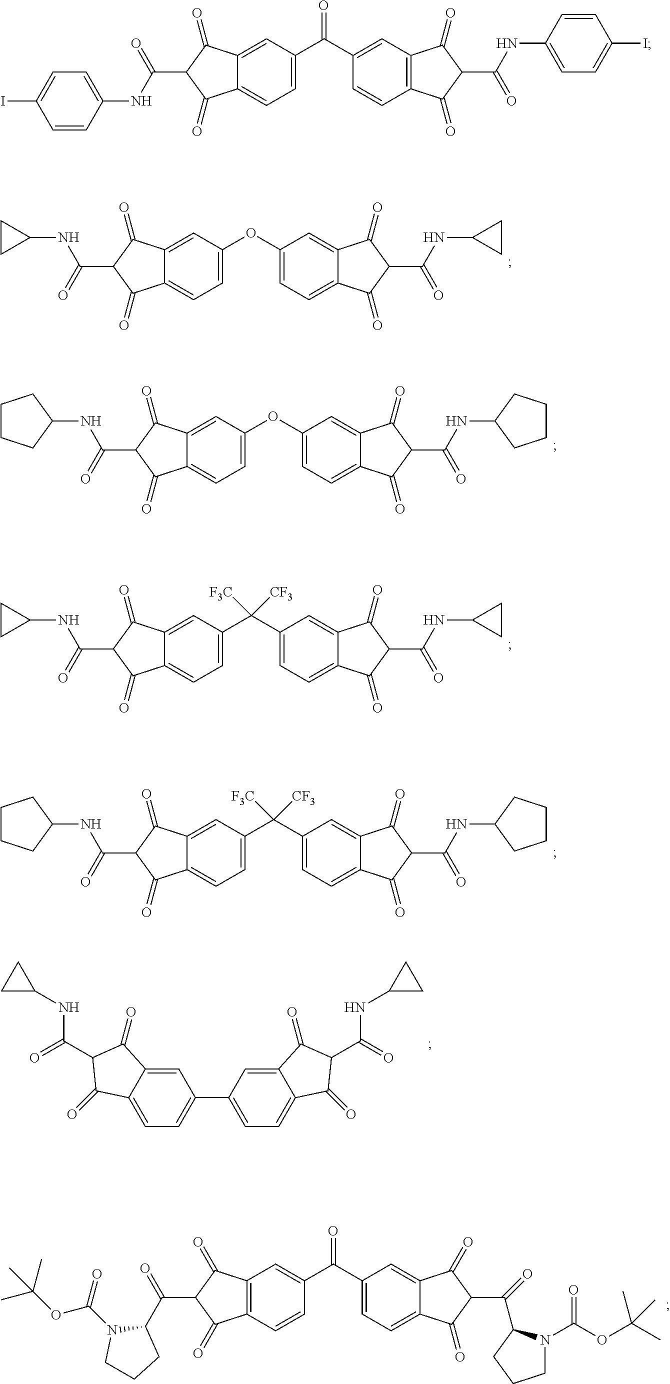

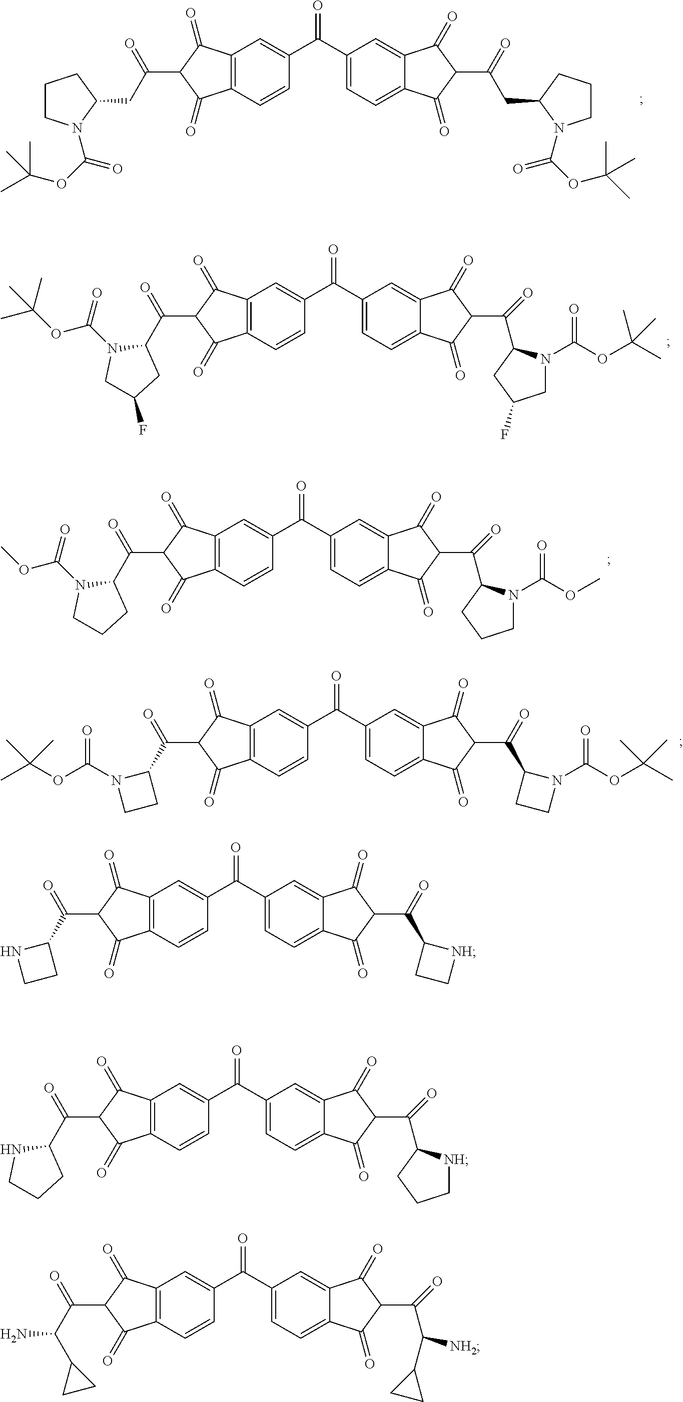

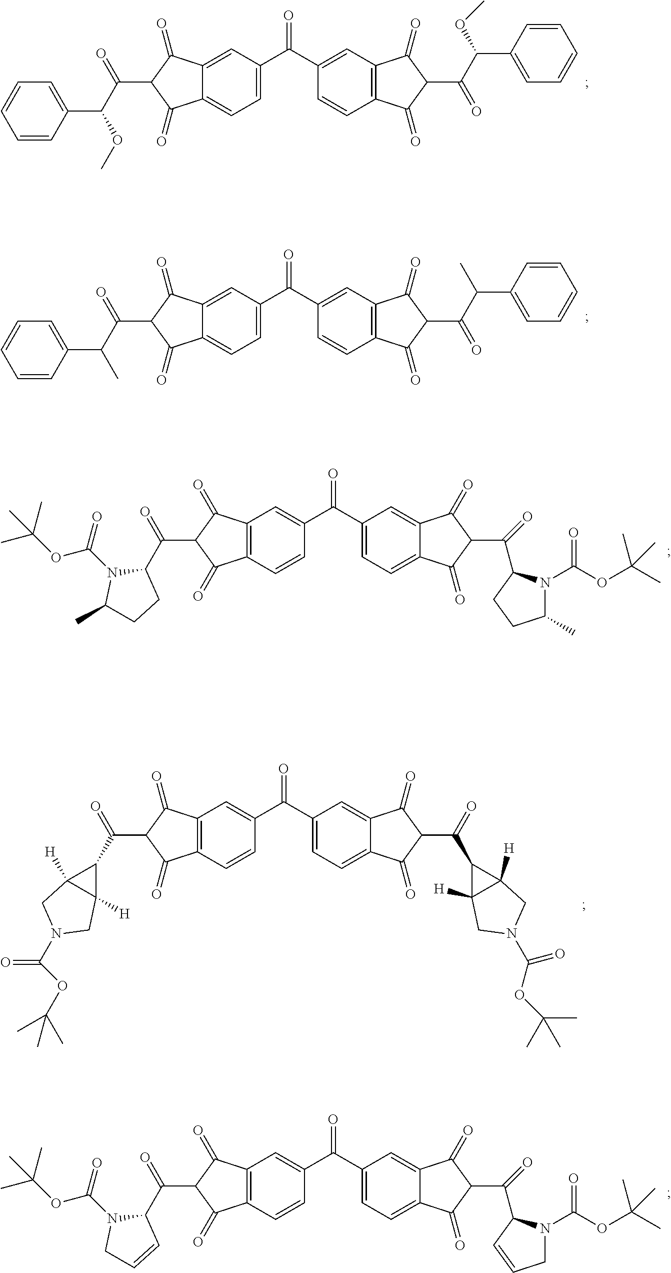

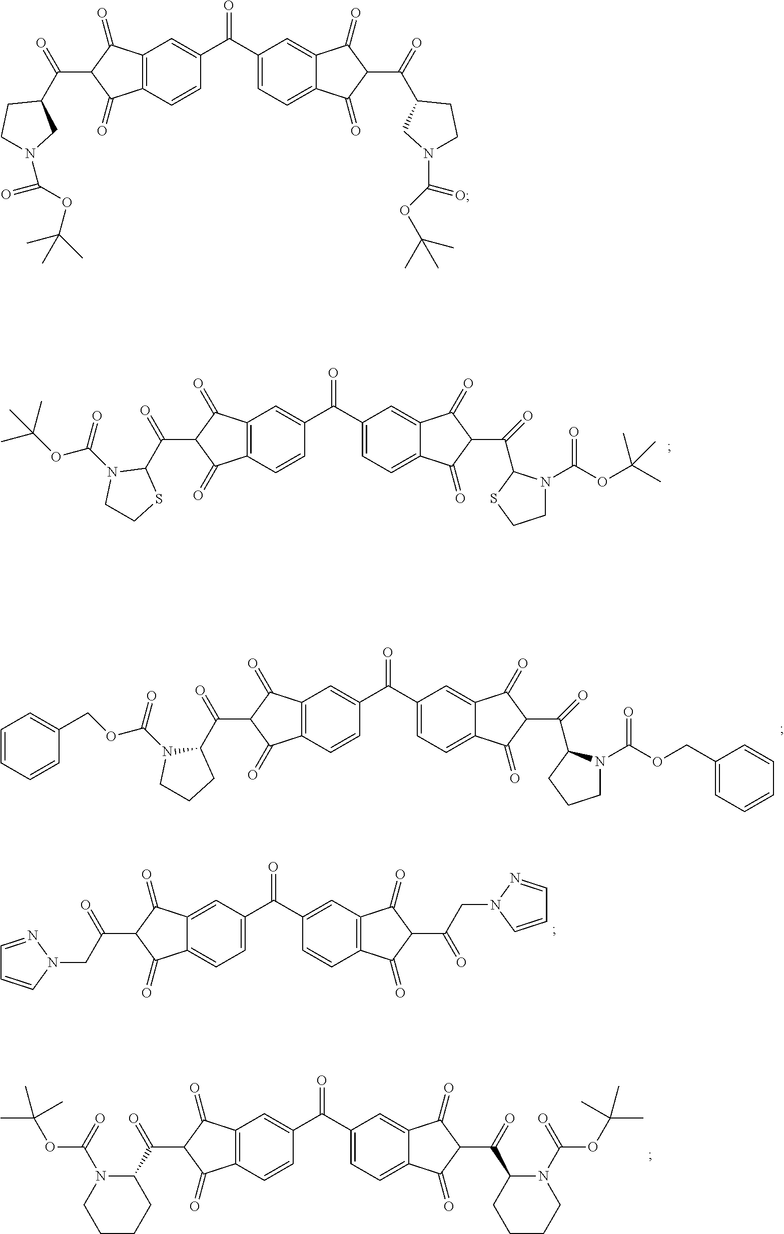

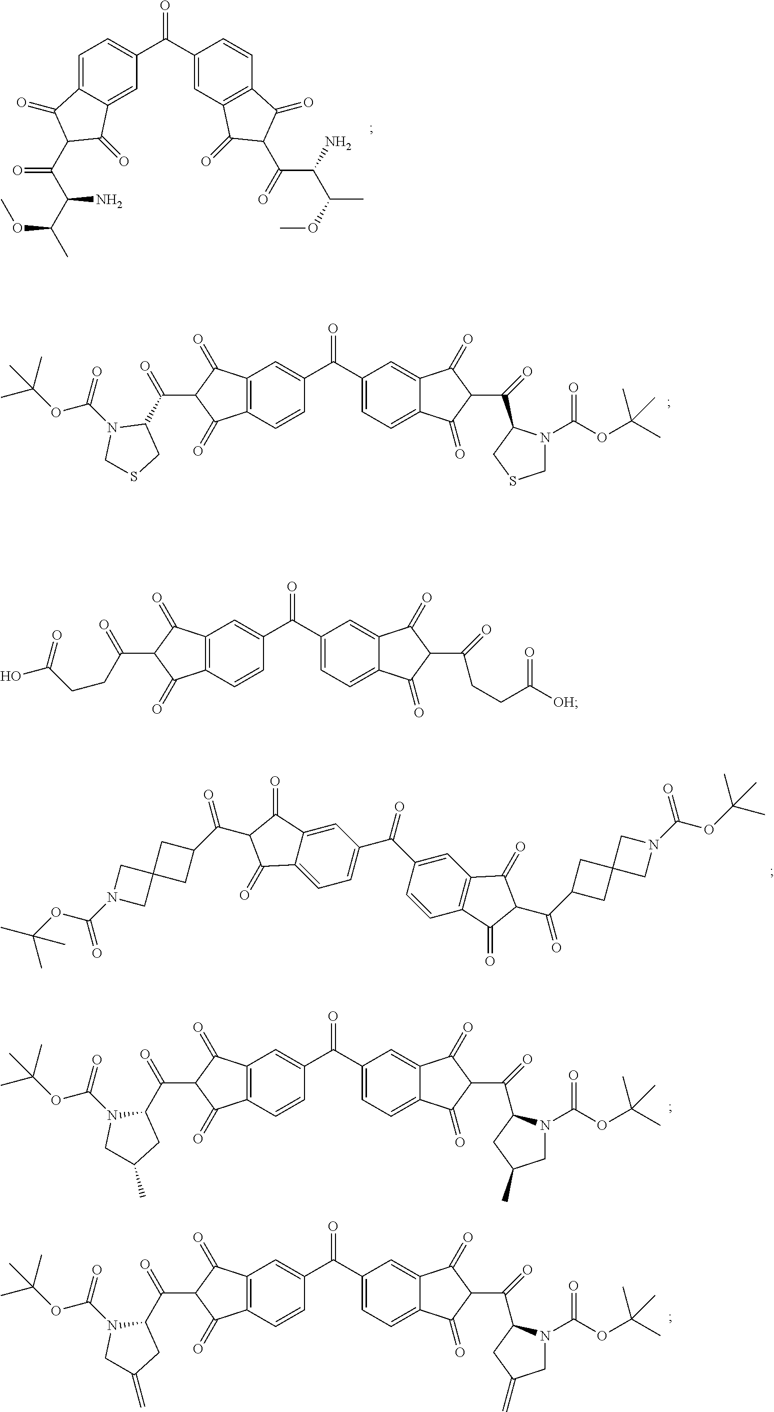

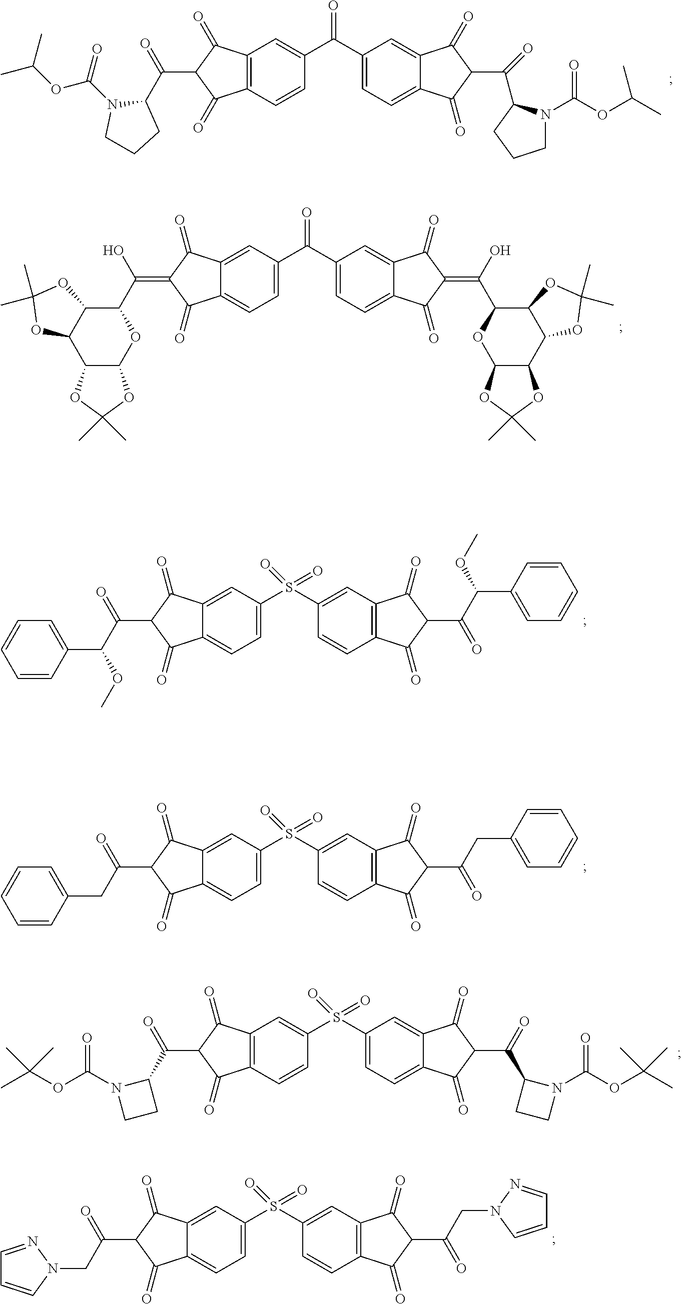

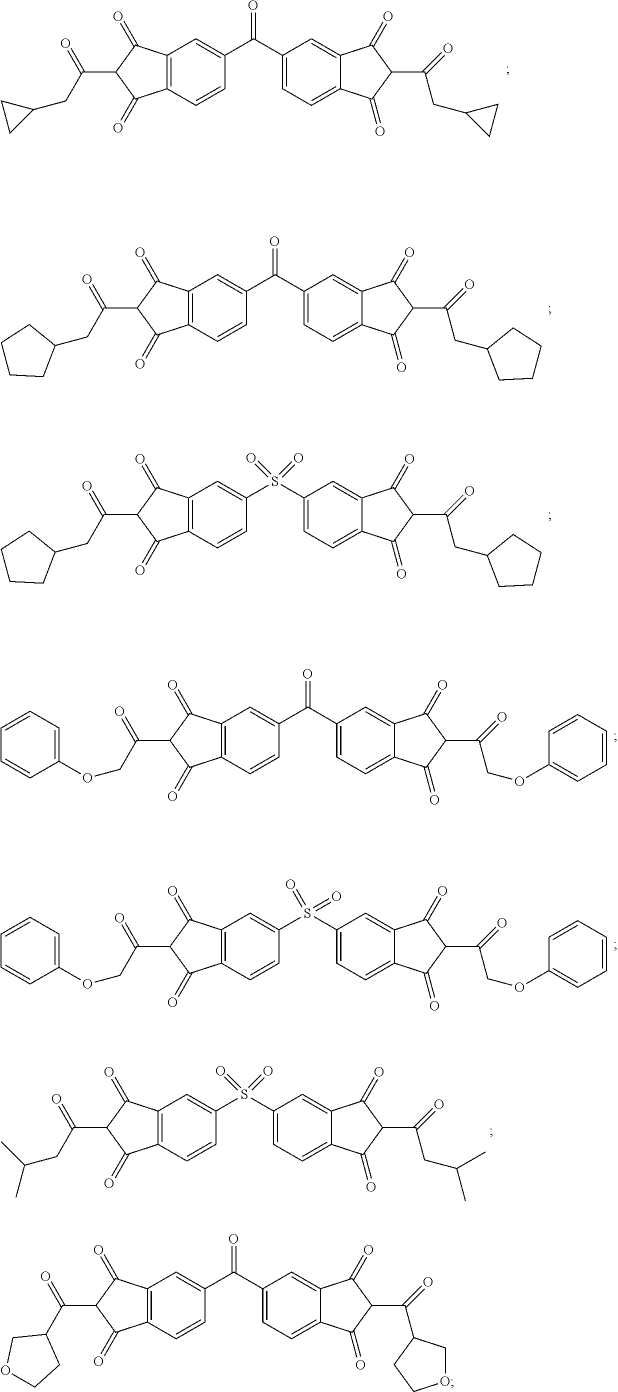

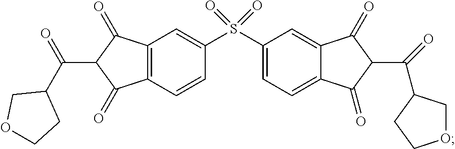 [SMILES: O=C1c2ccc(S(=O)(=O)c3ccc4c(c3)C(=O)C(C(=O)C3CCOC3)C4=O)cc2C(=O)C1C(=O)C1CCOC1]

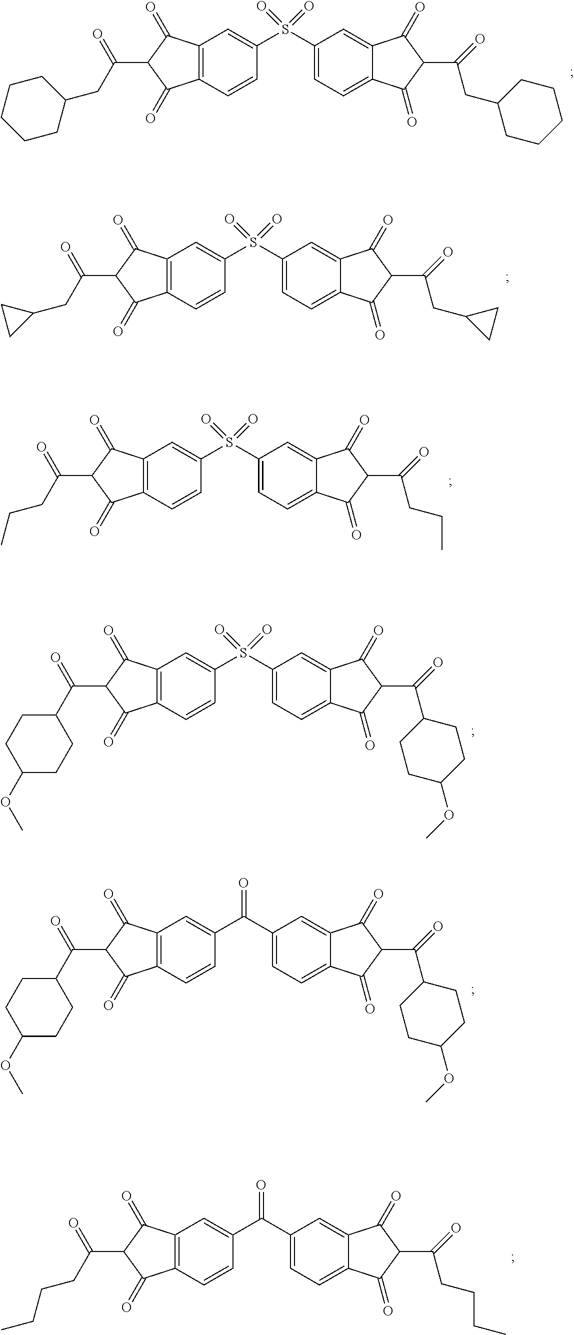

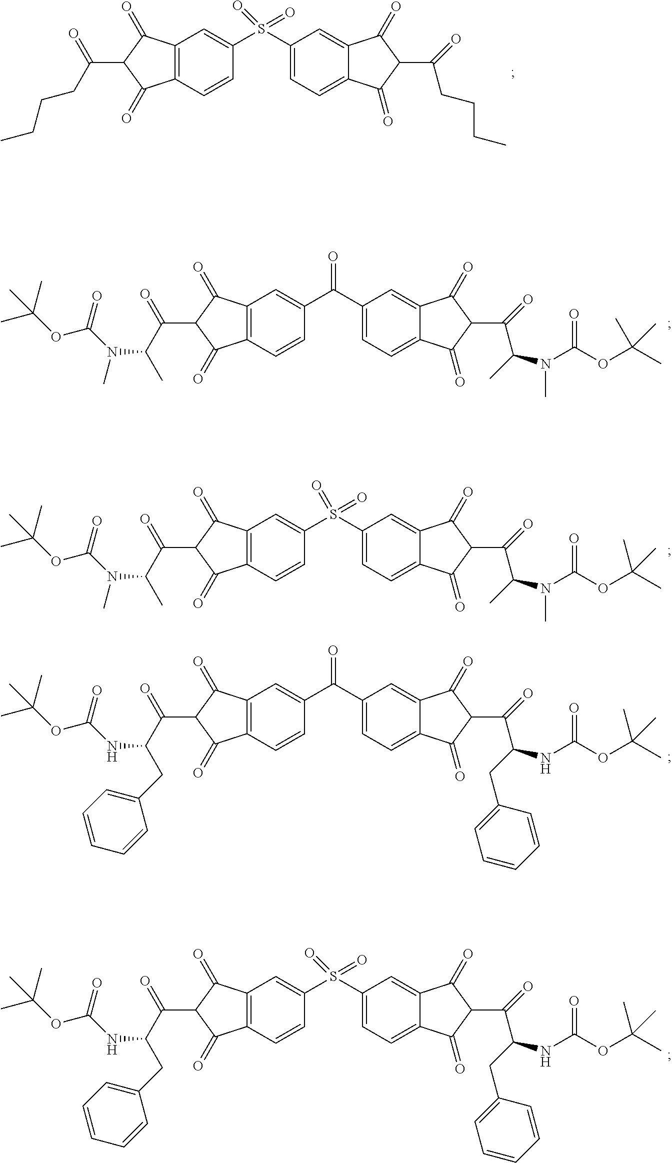

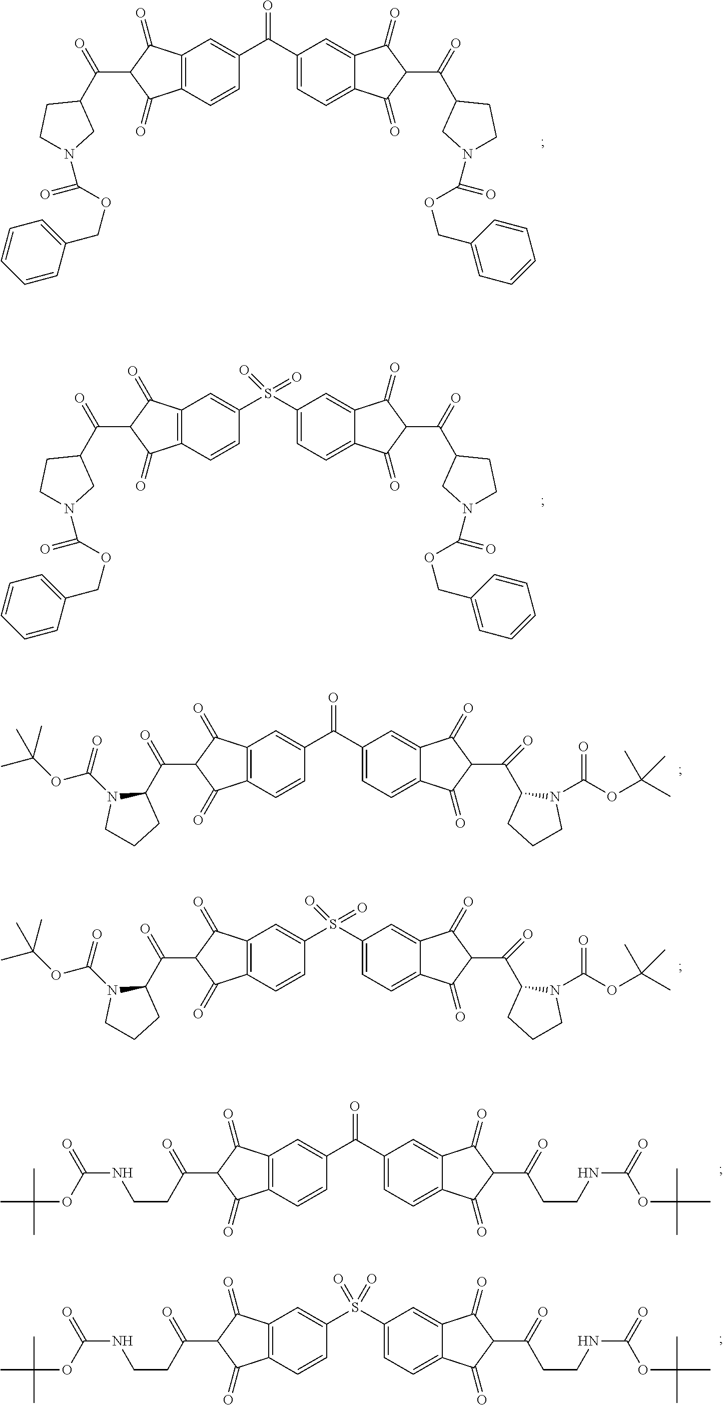

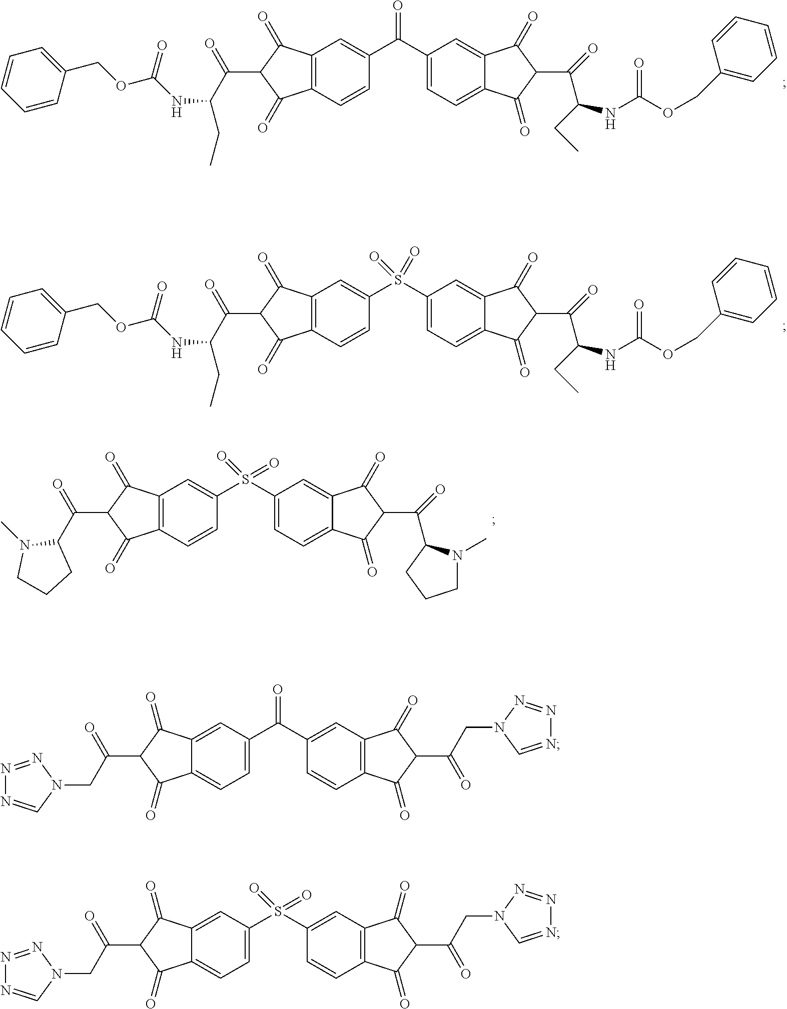

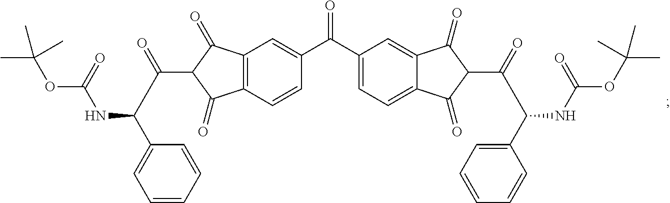 [SMILES: CC(C)(C)OC(=O)N[C@@H](C(=O)C1C(=O)c2ccc(C(=O)c3ccc4c(c3)C(=O)C(C(=O)[C@H](NC(=O)OC(C)(C)C)c3ccccc3)C4=O)cc2C1=O)c1ccccc1]

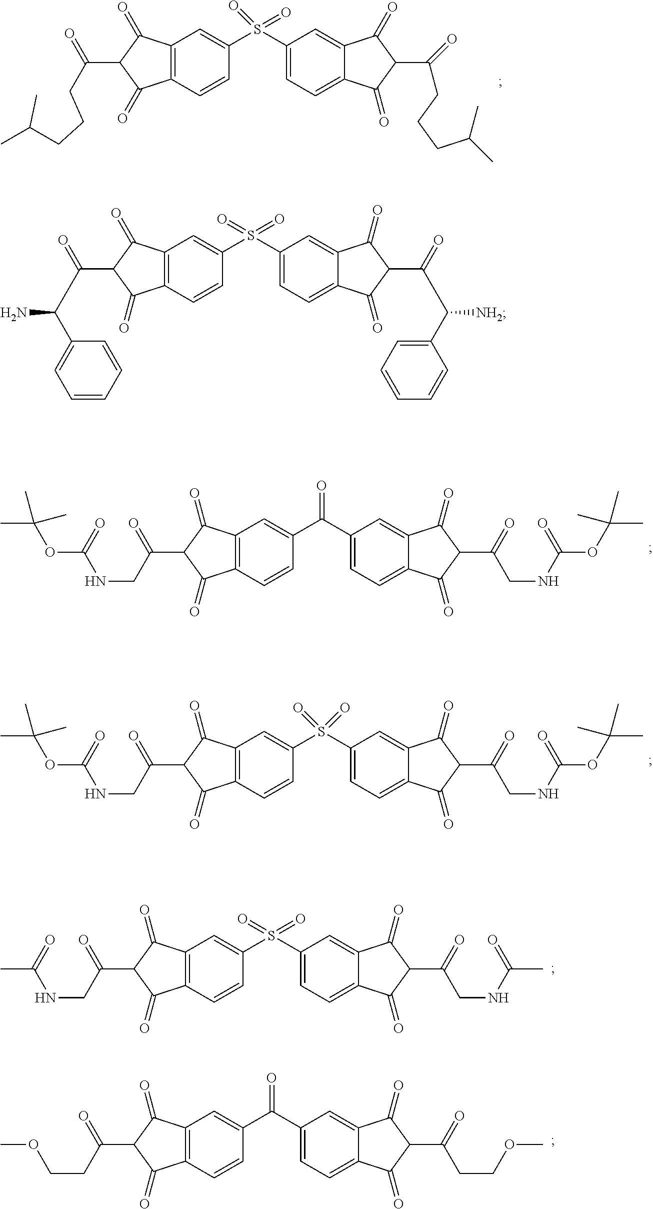

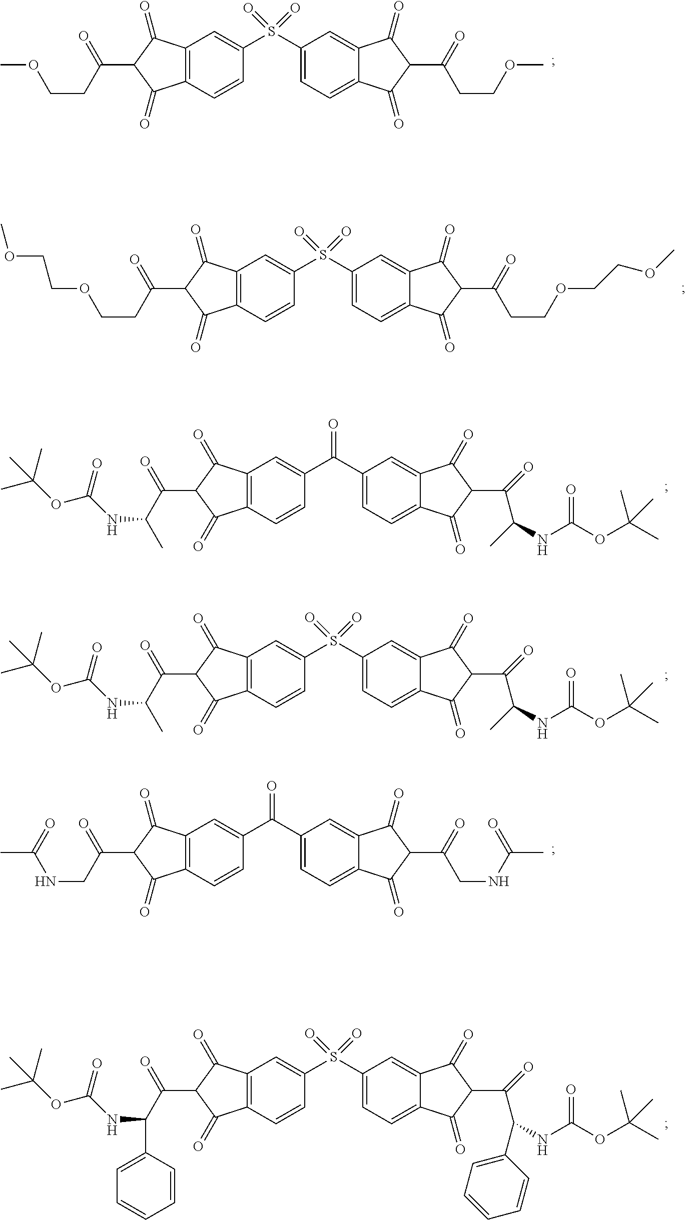

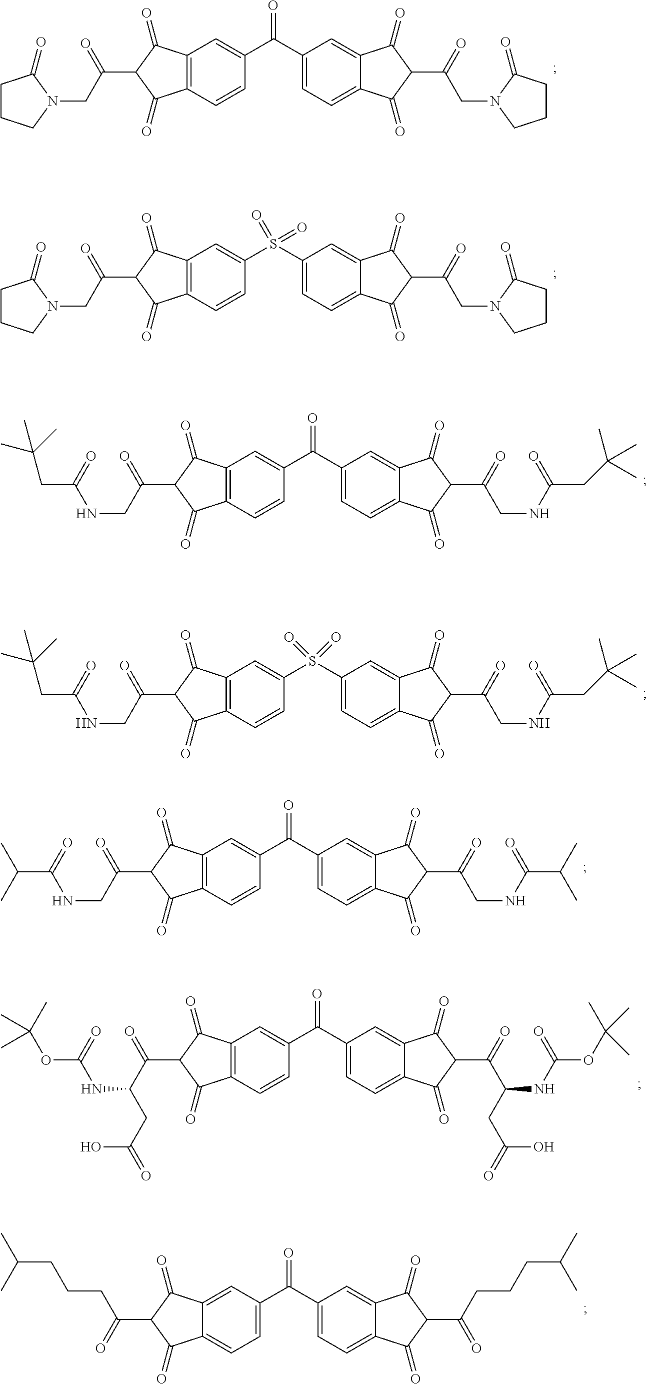

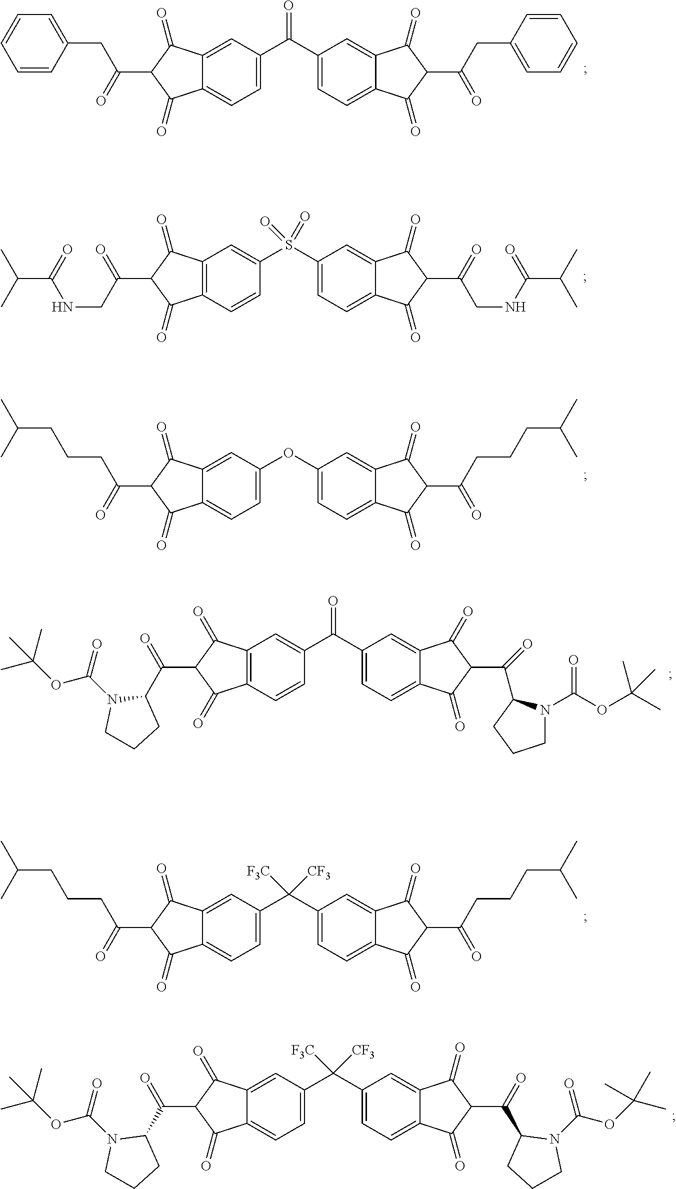

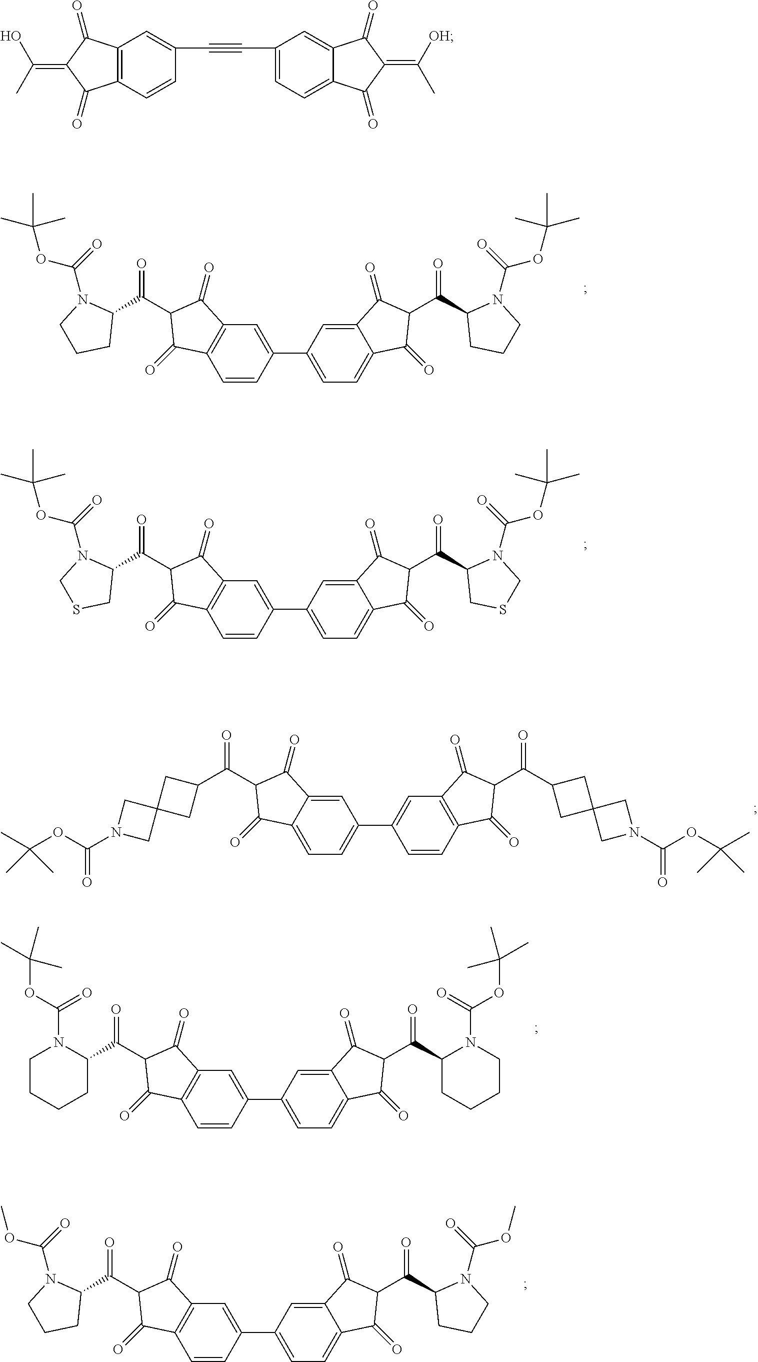

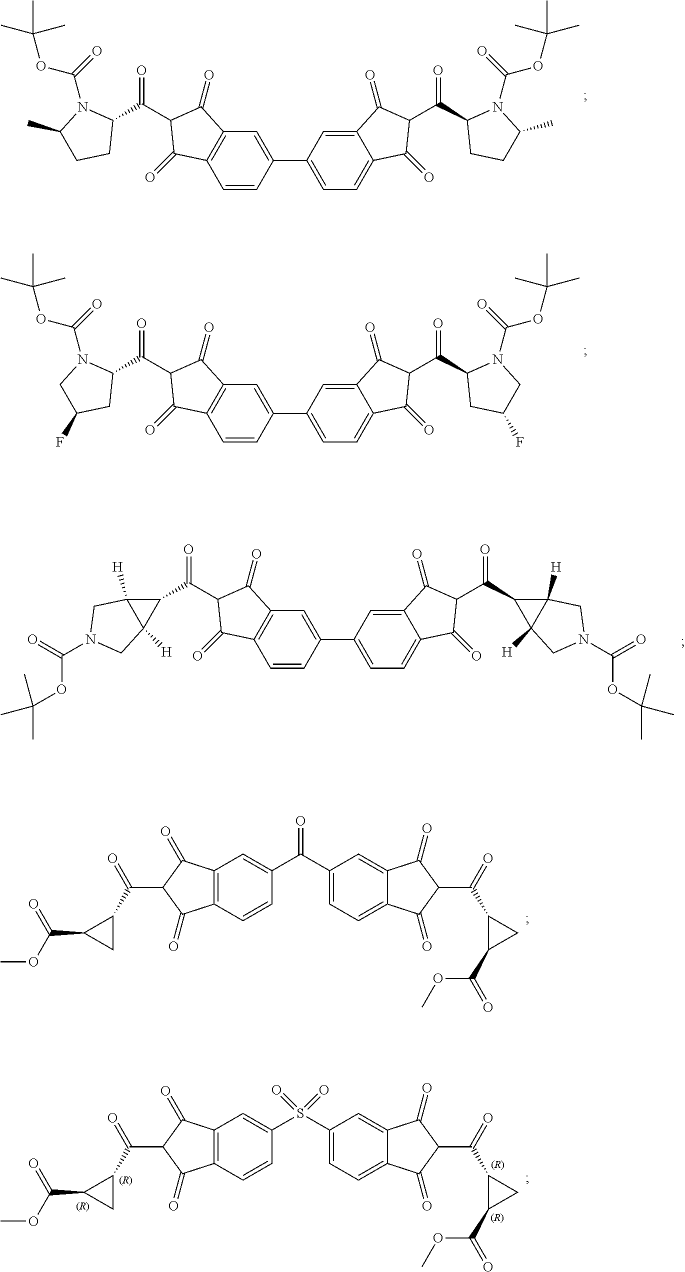

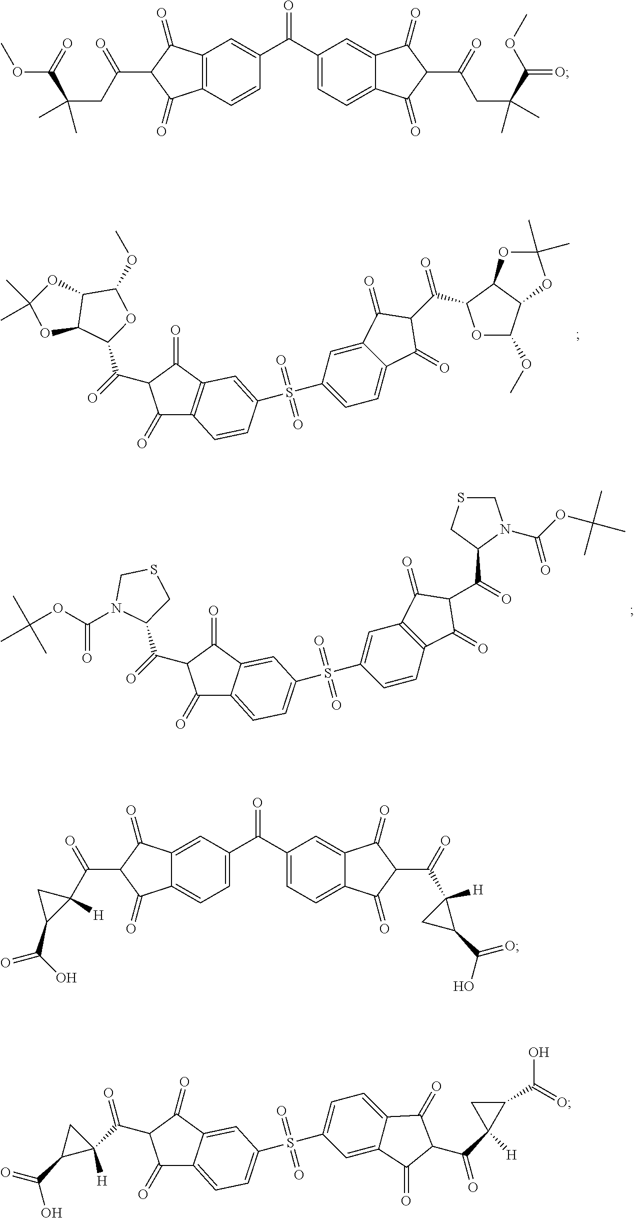

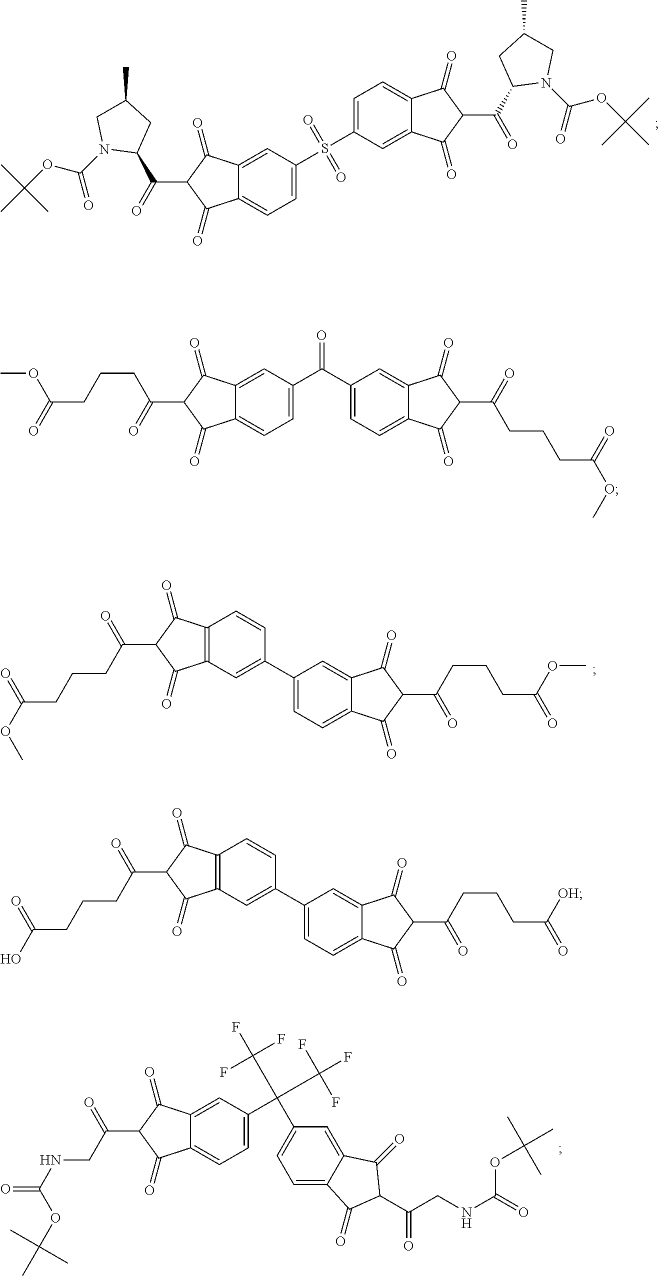

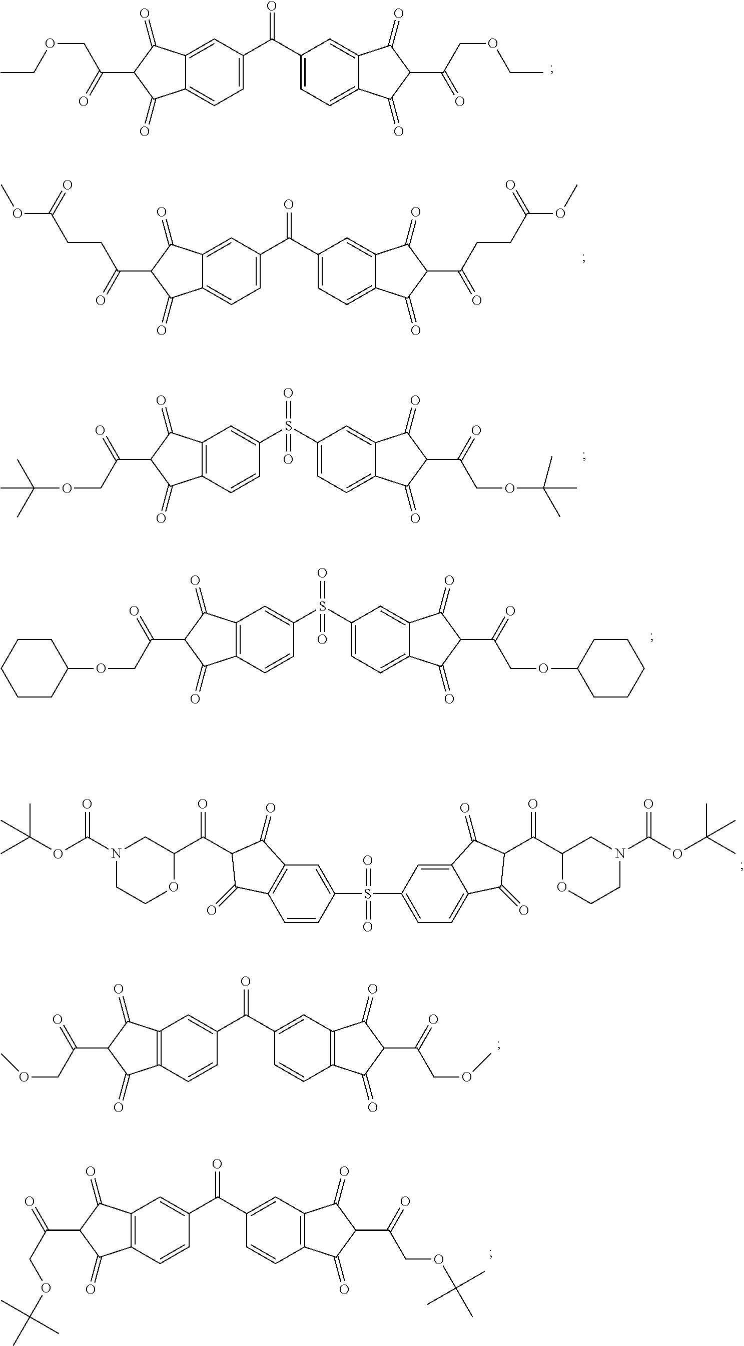

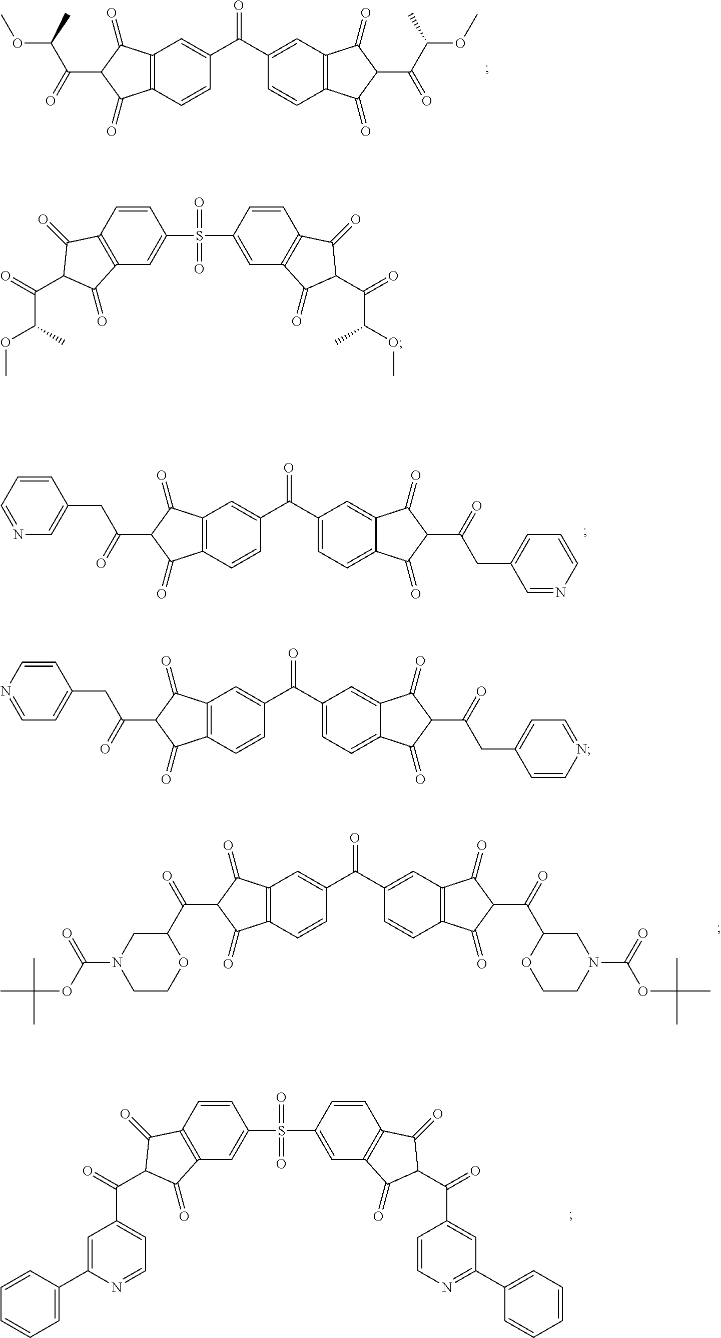

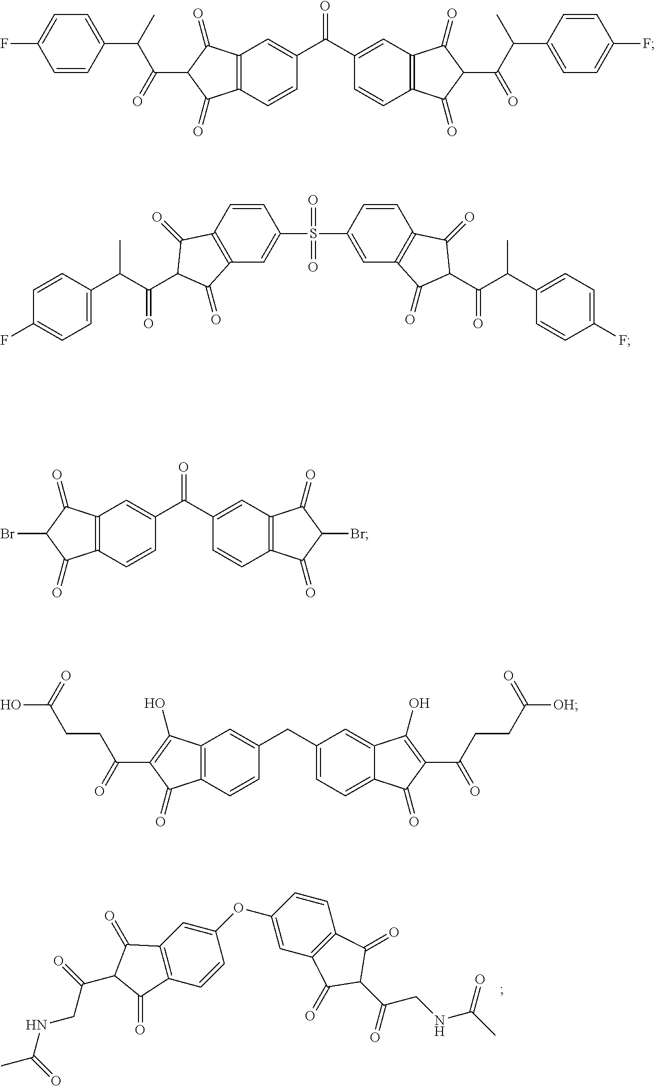 [SMILES: CC(C(=O)C1C(=O)c2ccc(C(=O)c3ccc4c(c3)C(=O)C(C(=O)C(C)c3ccc(F)cc3)C4=O)cc2C1=O)c1ccc(F)cc1 ; CC(C(=O)C1C(=O)c2ccc(S(=O)(=O)c3ccc4c(c3)C(=O)C(C(=O)C(C)c3ccc(F)cc3)C4=O)cc2C1=O)c1ccc(F)cc1 ; O=C(c1ccc2c(c1)C(=O)C(Br)C2=O)c1ccc2c(c1)C(=O)C(Br)C2=O ; O=C(O)CCC(=O)C1=C(O)c2cc(Cc3ccc4c(c3)C(O)=C(C(=O)CCC(=O)O)C4=O)ccc2C1=O ; CC(=O)NCC(=O)C1C(=O)c2ccc(Oc3ccc4c(c3)C(=O)C(C(=O)CNC(C)=O)C4=O)cc2C1=O]

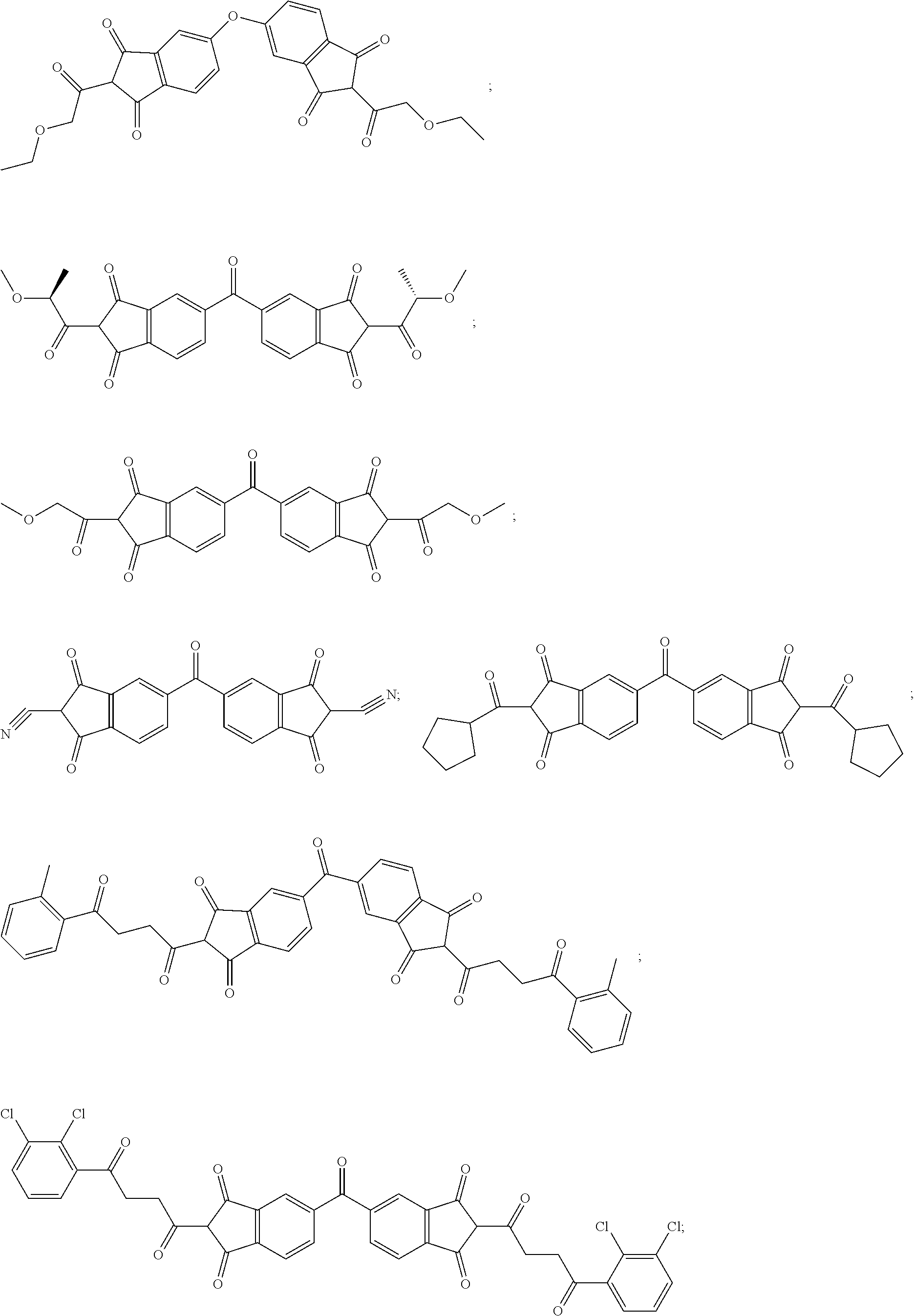

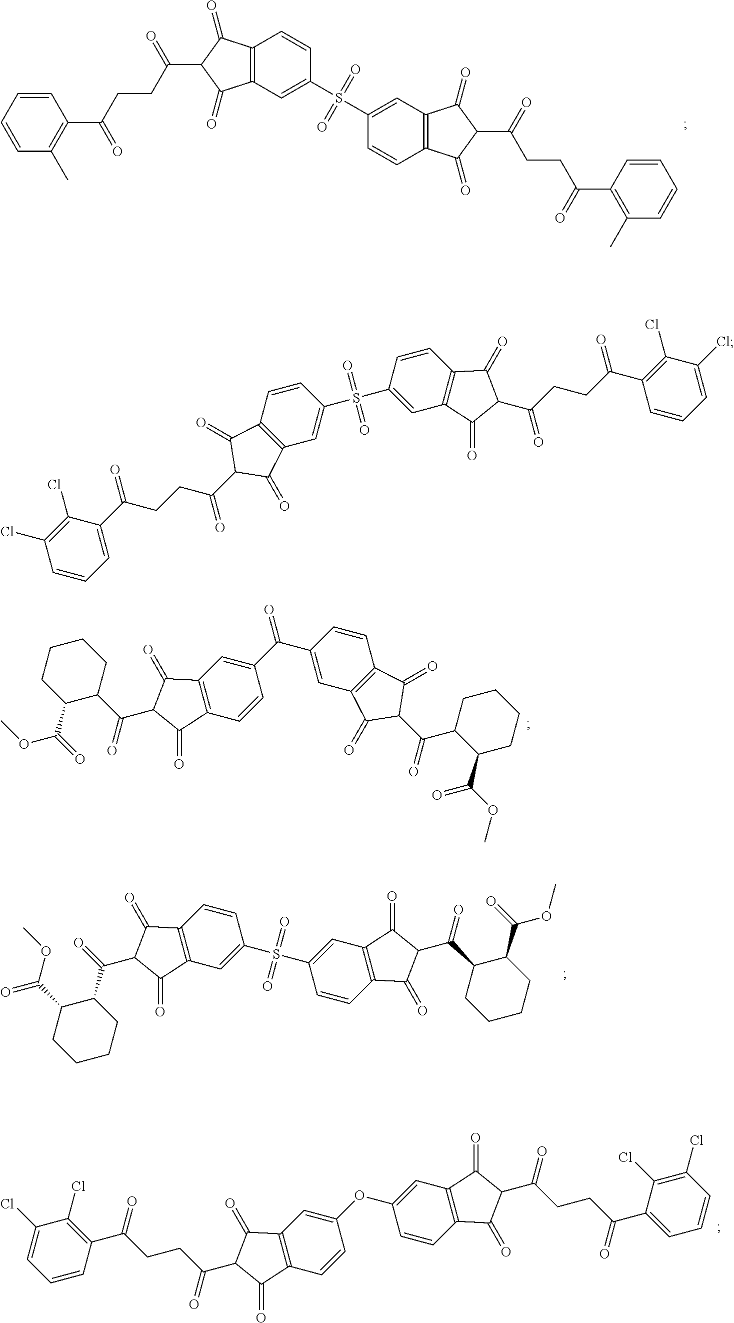

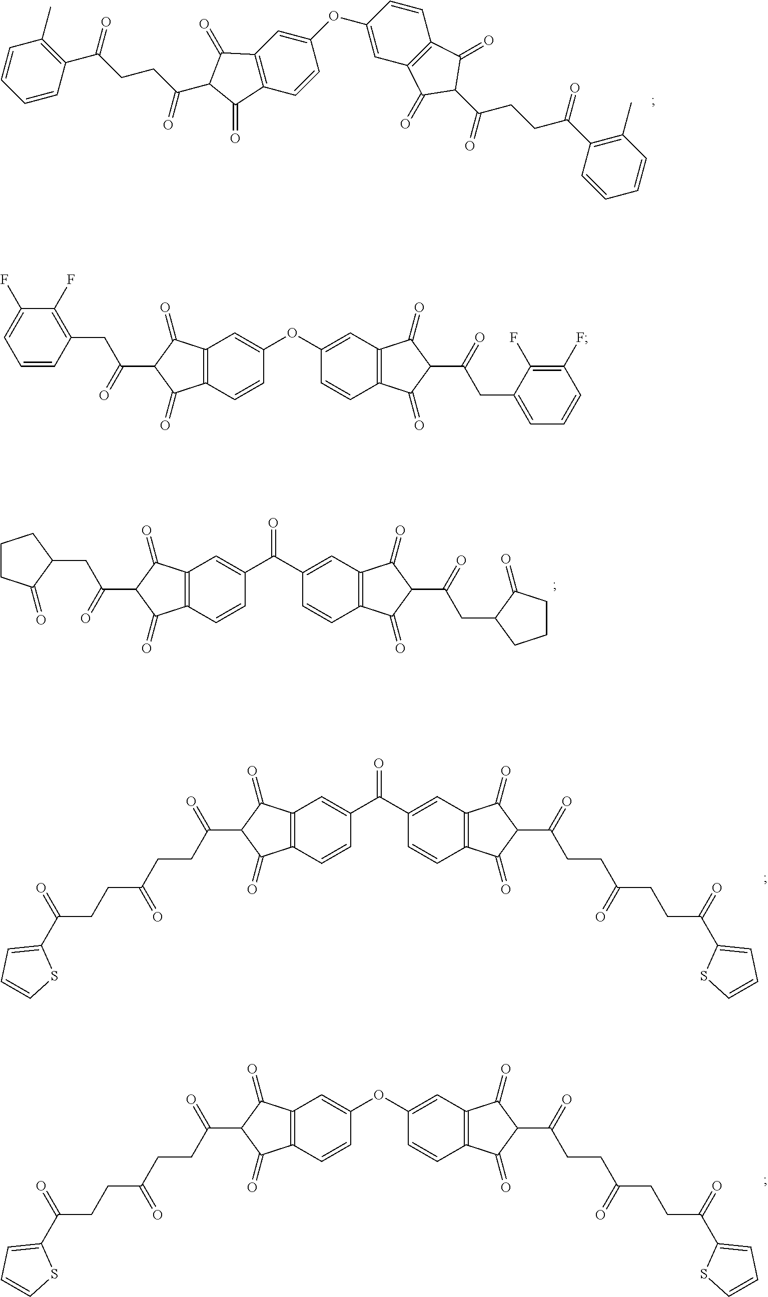 [SMILES: Cc1ccccc1C(=O)CCC(=O)C1C(=O)c2ccc(Oc3ccc4c(c3)C(=O)C(C(=O)CCC(=O)c3ccccc3C)C4=O)cc2C1=O ; O=C(Cc1cccc(F)c1F)C1C(=O)c2ccc(Oc3ccc4c(c3)C(=O)C(C(=O)Cc3cccc(F)c3F)C4=O)cc2C1=O ; O=C(c1ccc2c(c1)C(=O)C(C(=O)CC1CCCC1=O)C2=O)c1ccc2c(c1)C(=O)C(C(=O)CC1CCCC1=O)C2=O ; O=C(CCC(=O)c1cccs1)CCC(=O)C1C(=O)c2ccc(C(=O)c3ccc4c(c3)C(=O)C(C(=O)CCC(=O)CCC(=O)c3cccs3)C4=O)cc2C1=O ; O=C(CCC(=O)c1cccs1)CCC(=O)C1C(=O)c2ccc(Oc3ccc4c(c3)C(=O)C(C(=O)CCC(=O)CCC(=O)c3cccs3)C4=O)cc2C1=O]

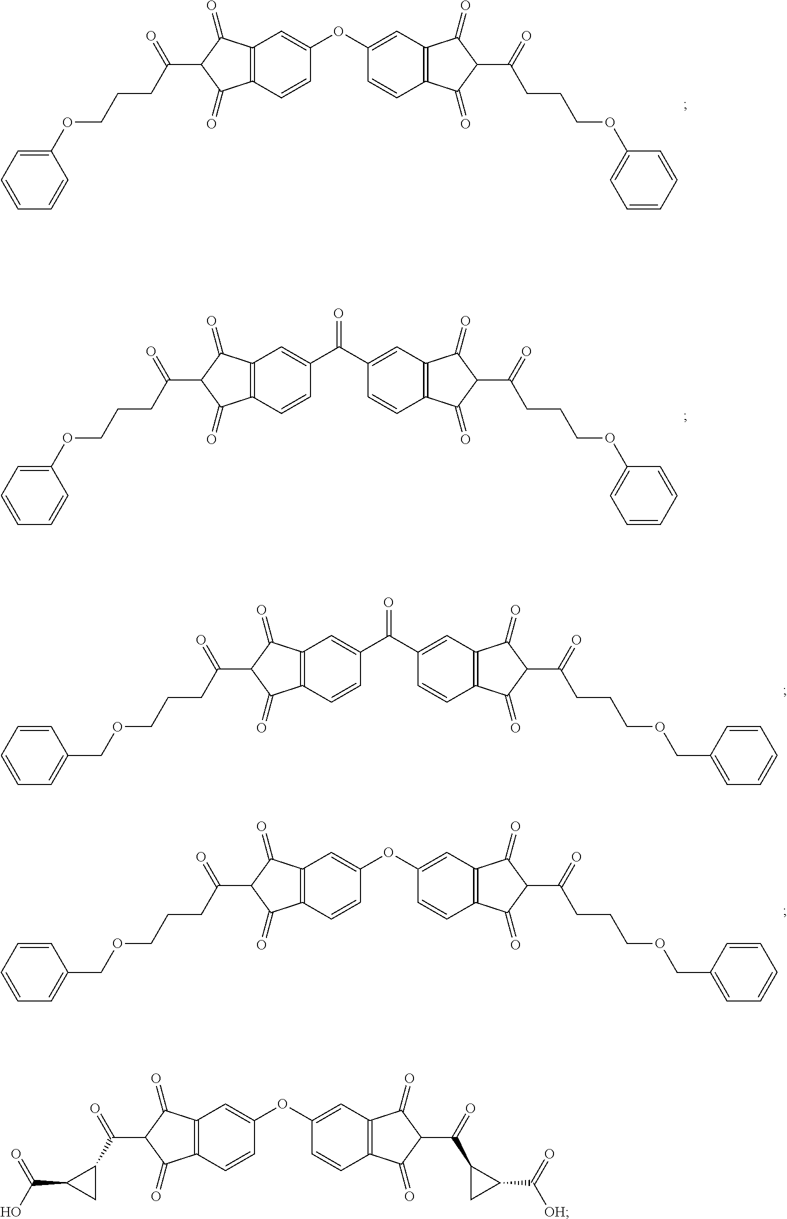 [SMILES: O=C(CCCOc1ccccc1)C1C(=O)c2ccc(Oc3ccc4c(c3)C(=O)C(C(=O)CCCOc3ccccc3)C4=O)cc2C1=O ; O=C(c1ccc2c(c1)C(=O)C(C(=O)CCCOc1ccccc1)C2=O)c1ccc2c(c1)C(=O)C(C(=O)CCCOc1ccccc1)C2=O ; O=C(c1ccc2c(c1)C(=O)C(C(=O)CCCOCc1ccccc1)C2=O)c1ccc2c(c1)C(=O)C(C(=O)CCCOCc1ccccc1)C2=O ; O=C(CCCOCc1ccccc1)C1C(=O)c2ccc(Oc3ccc4c(c3)C(=O)C(C(=O)CCCOCc3ccccc3)C4=O)cc2C1=O ; O=C1c2ccc(Oc3ccc4c(c3)C(=O)C(C(=O)[C@@H]3C[C@H]3C(=O)O)C4=O)cc2C(=O)C1C(=O)[C@@H]1C[C@H]1C(=O)O]

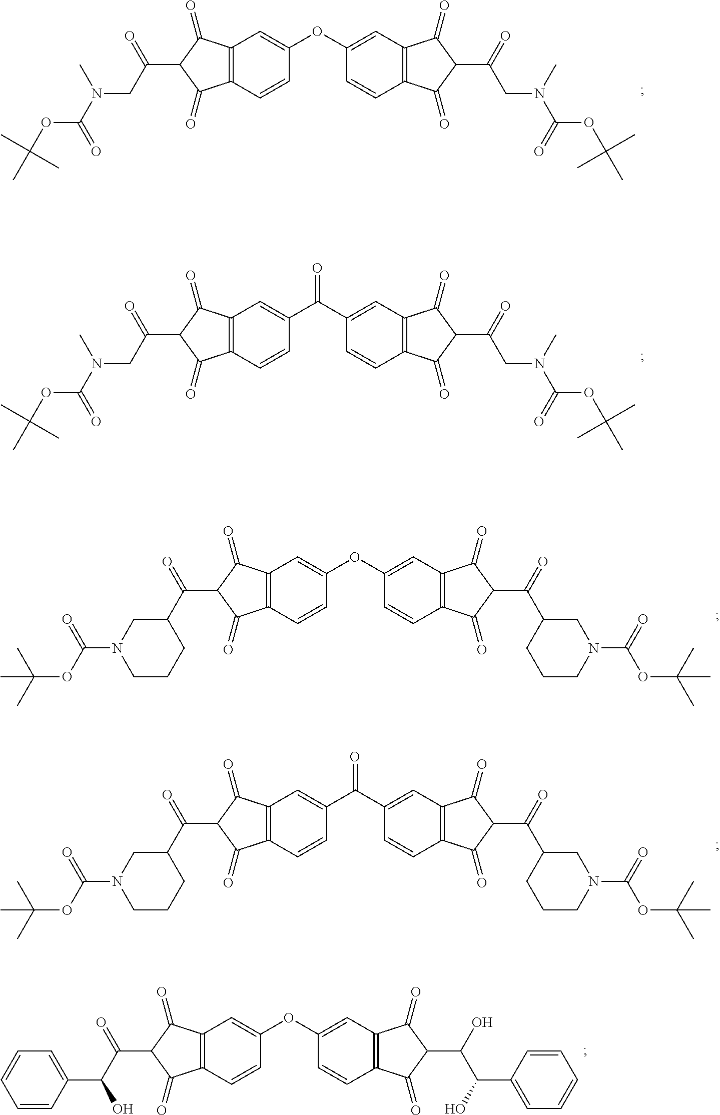

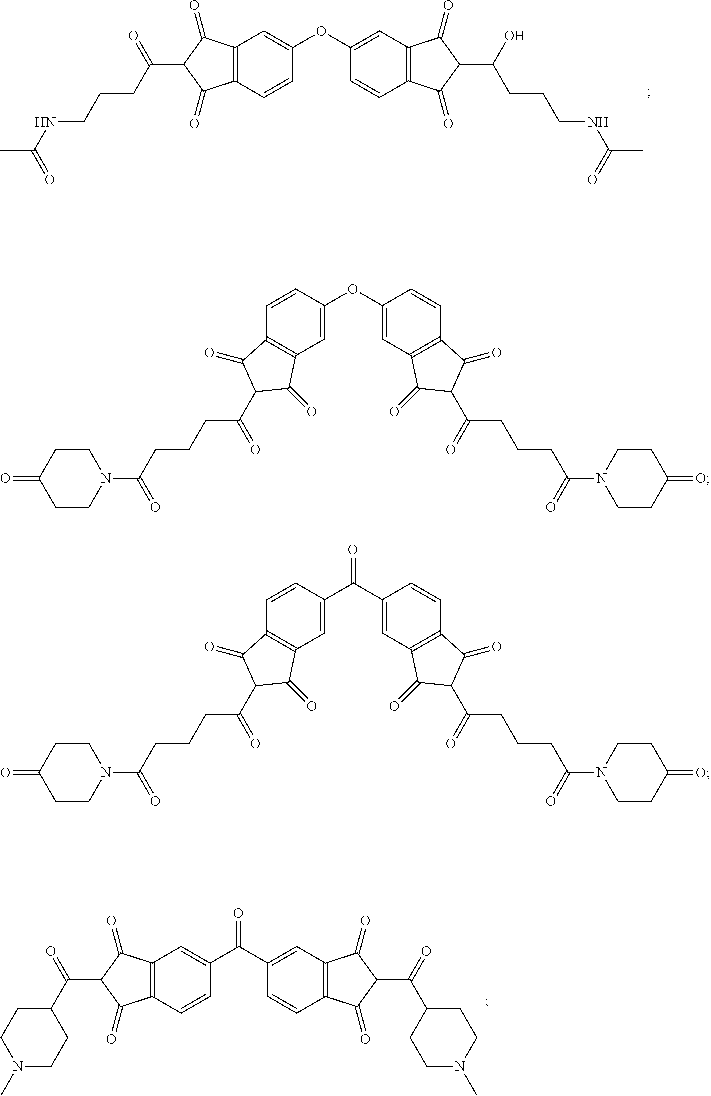 [SMILES: CC(=O)NCCCC(=O)C1C(=O)c2ccc(Oc3ccc4c(c3)C(=O)C(C(O)CCCNC(C)=O)C4=O)cc2C1=O ; O=C1CCN(C(=O)CCCC(=O)C2C(=O)c3ccc(Oc4ccc5c(c4)C(=O)C(C(=O)CCCC(=O)N4CCC(=O)CC4)C5=O)cc3C2=O)CC1 ; O=C1CCN(C(=O)CCCC(=O)C2C(=O)c3ccc(C(=O)c4ccc5c(c4)C(=O)C(C(=O)CCCC(=O)N4CCC(=O)CC4)C5=O)cc3C2=O)CC1 ; CN1CCC(C(=O)C2C(=O)c3ccc(C(=O)c4ccc5c(c4)C(=O)C(C(=O)C4CCN(C)CC4)C5=O)cc3C2=O)CC1]

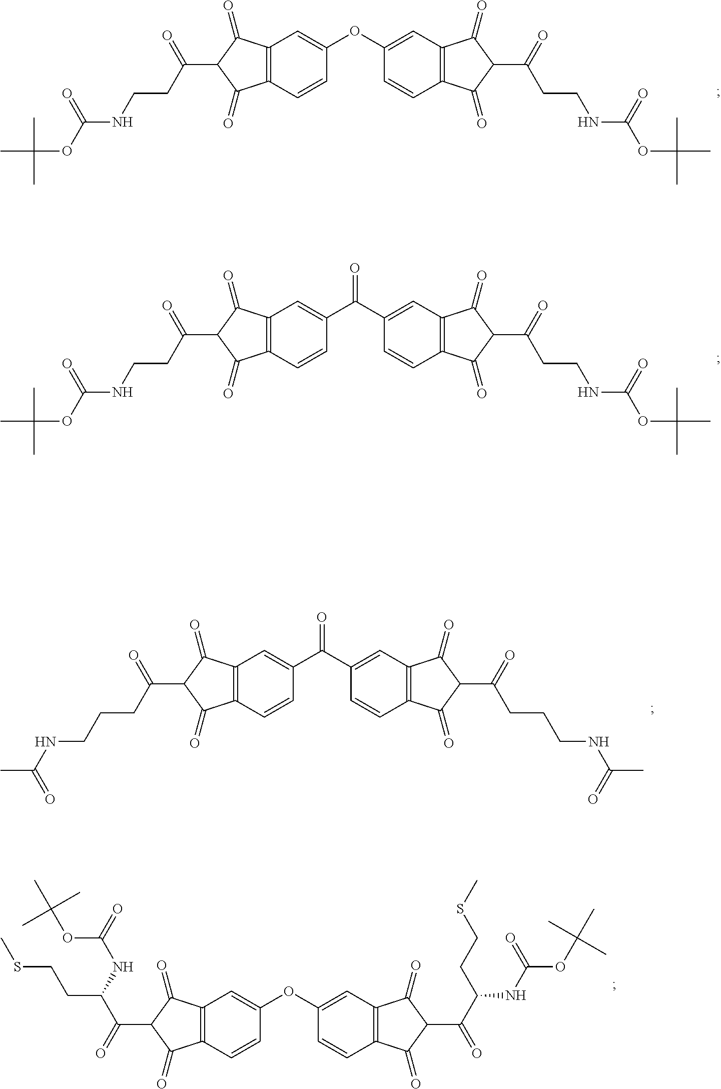

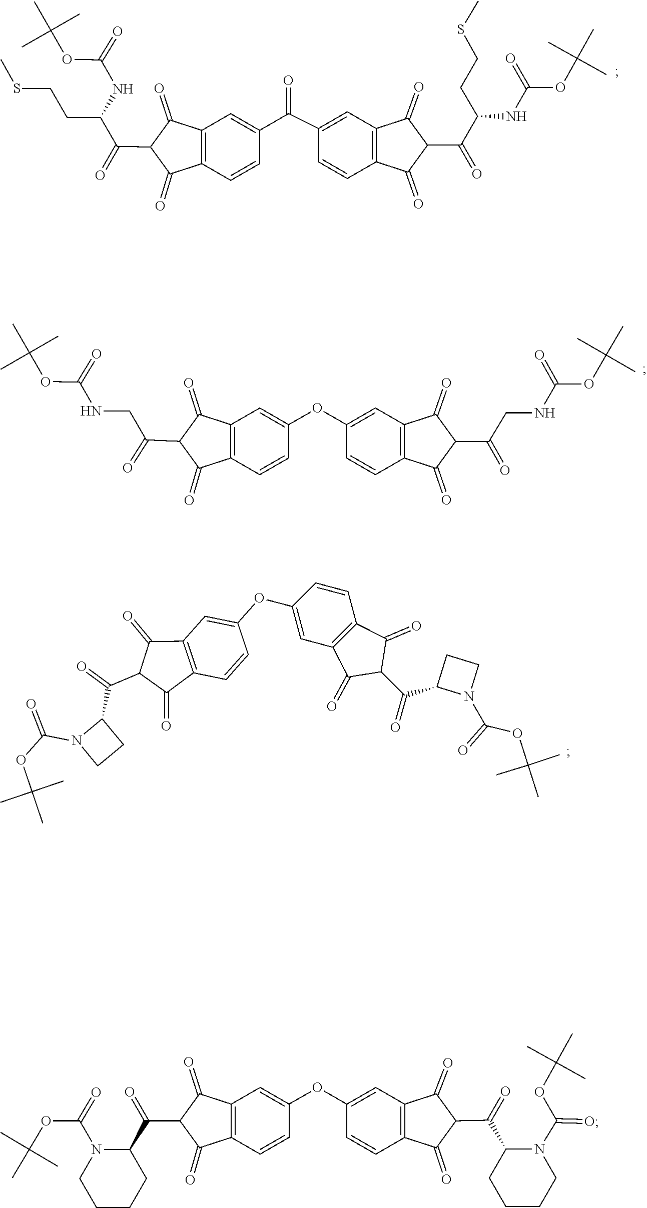

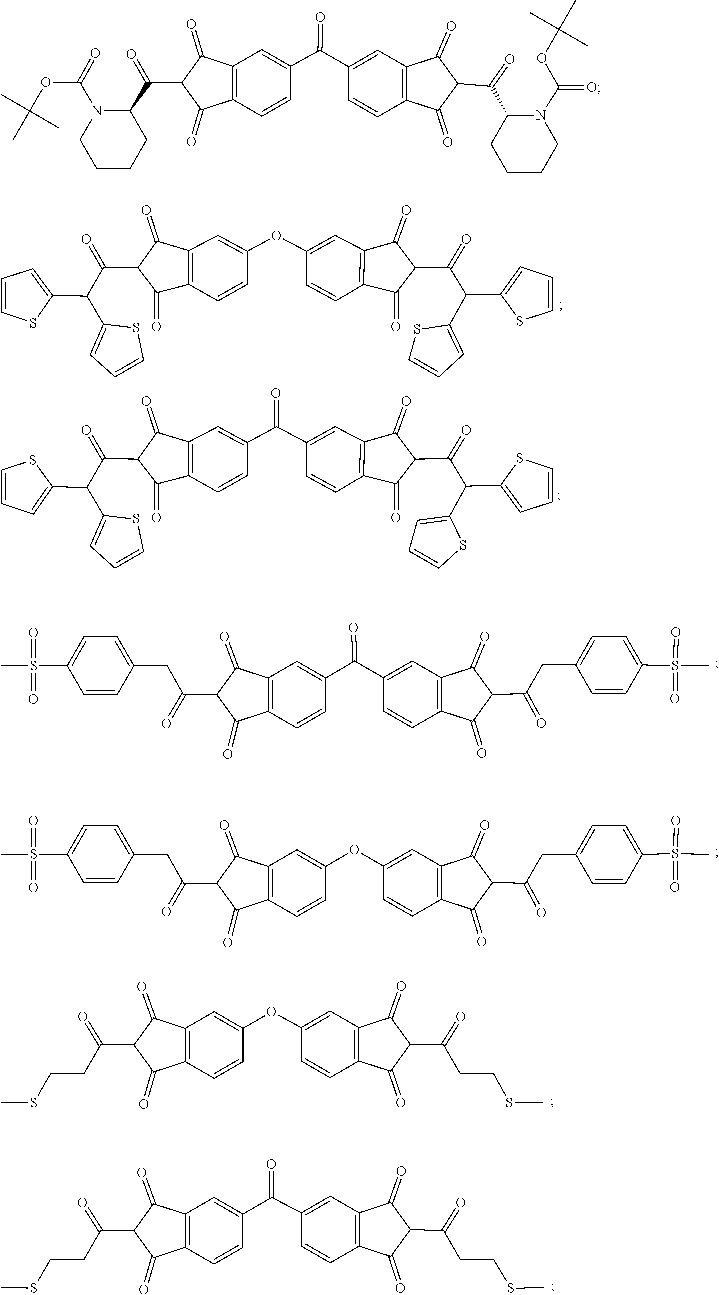

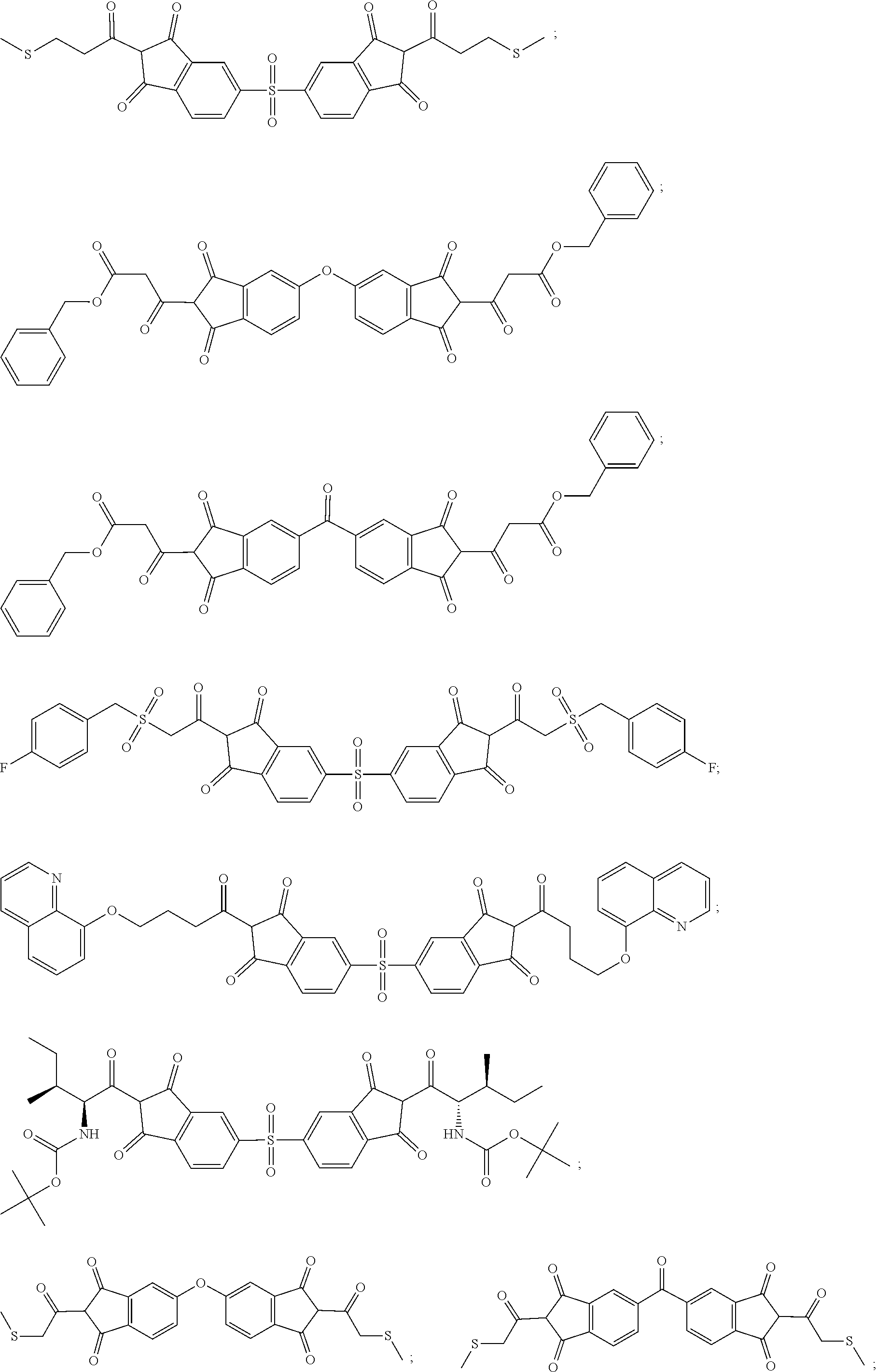

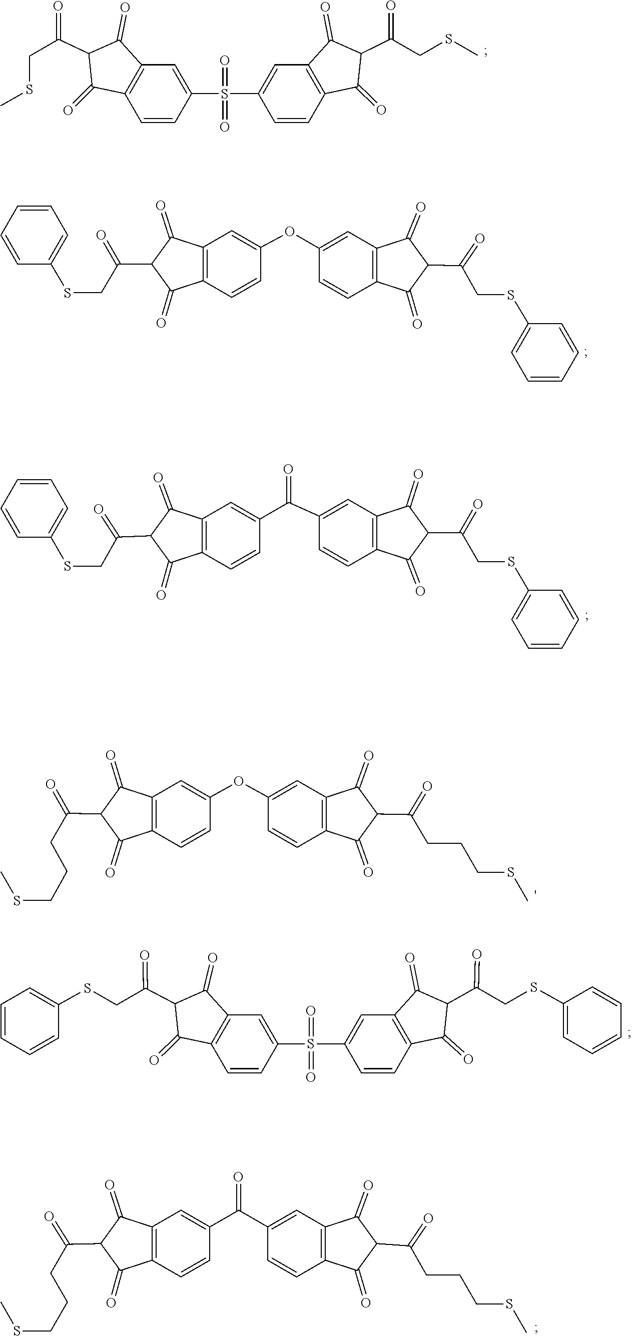

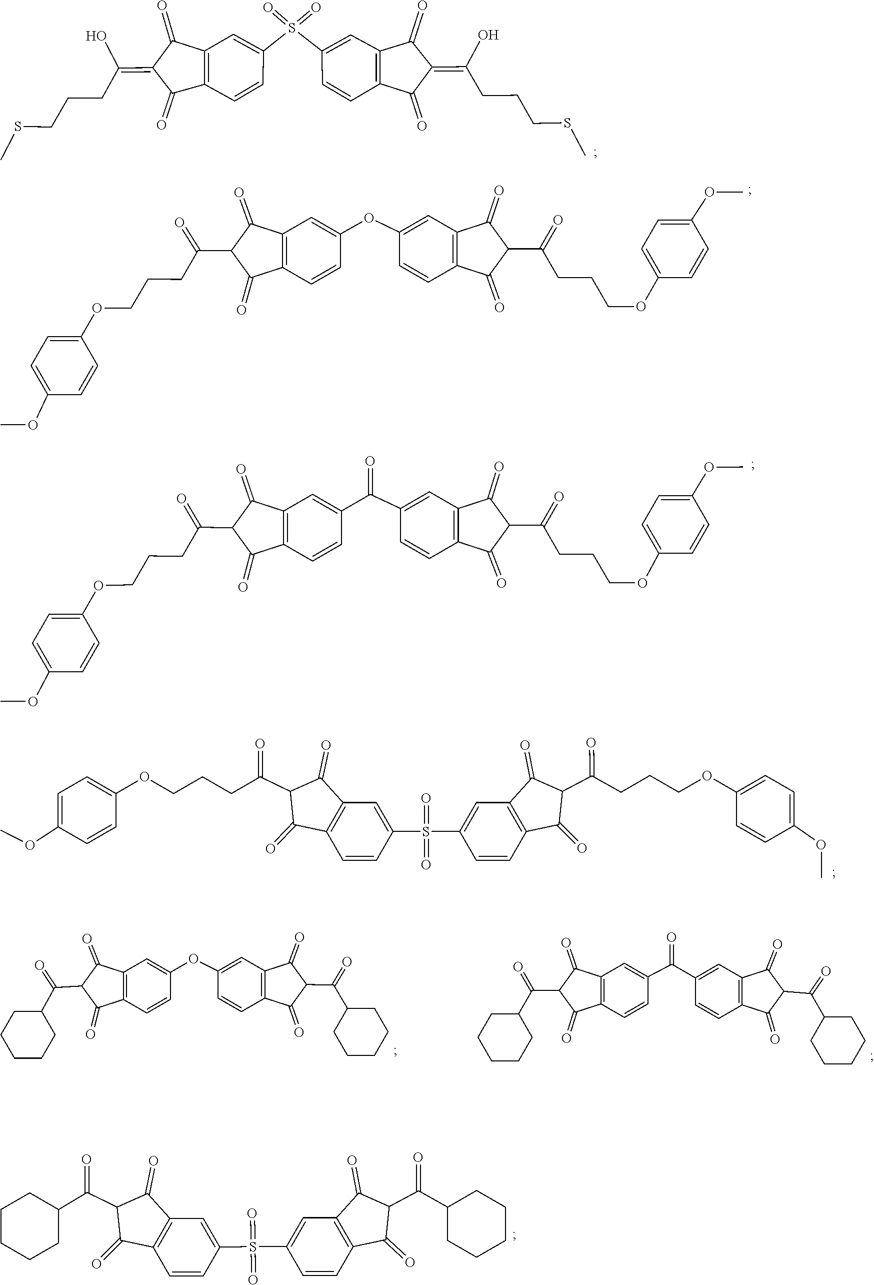

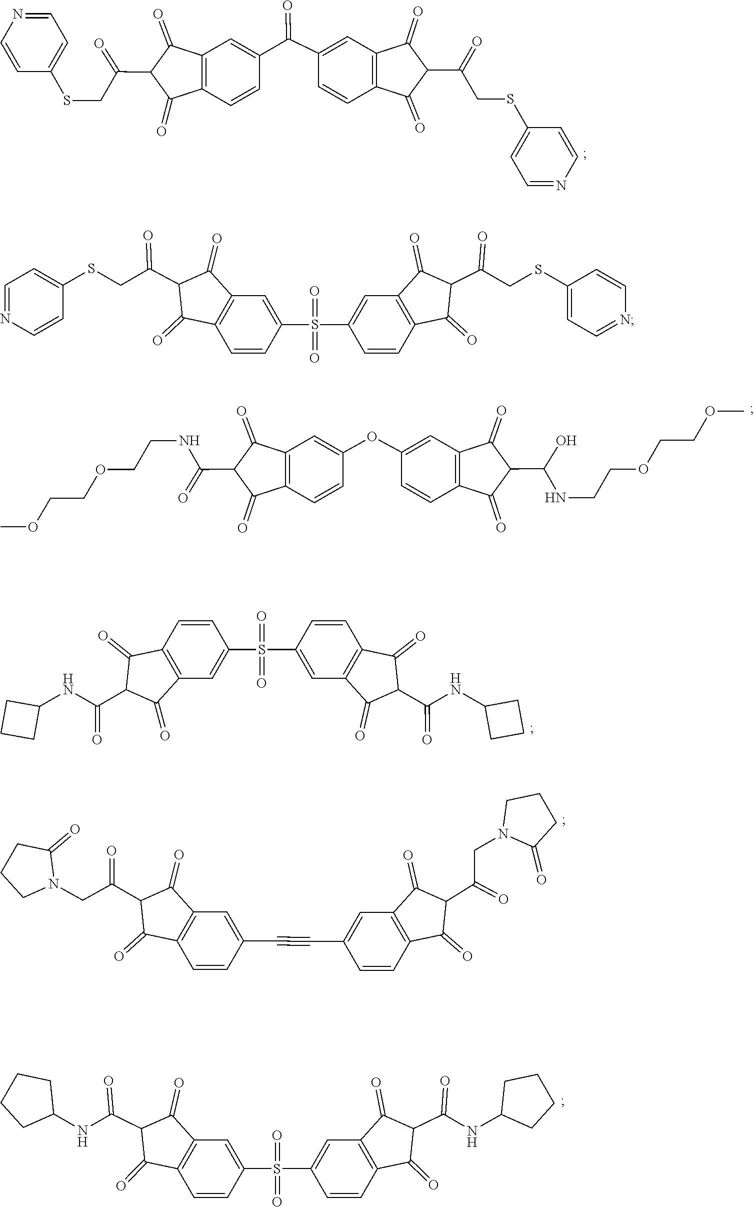

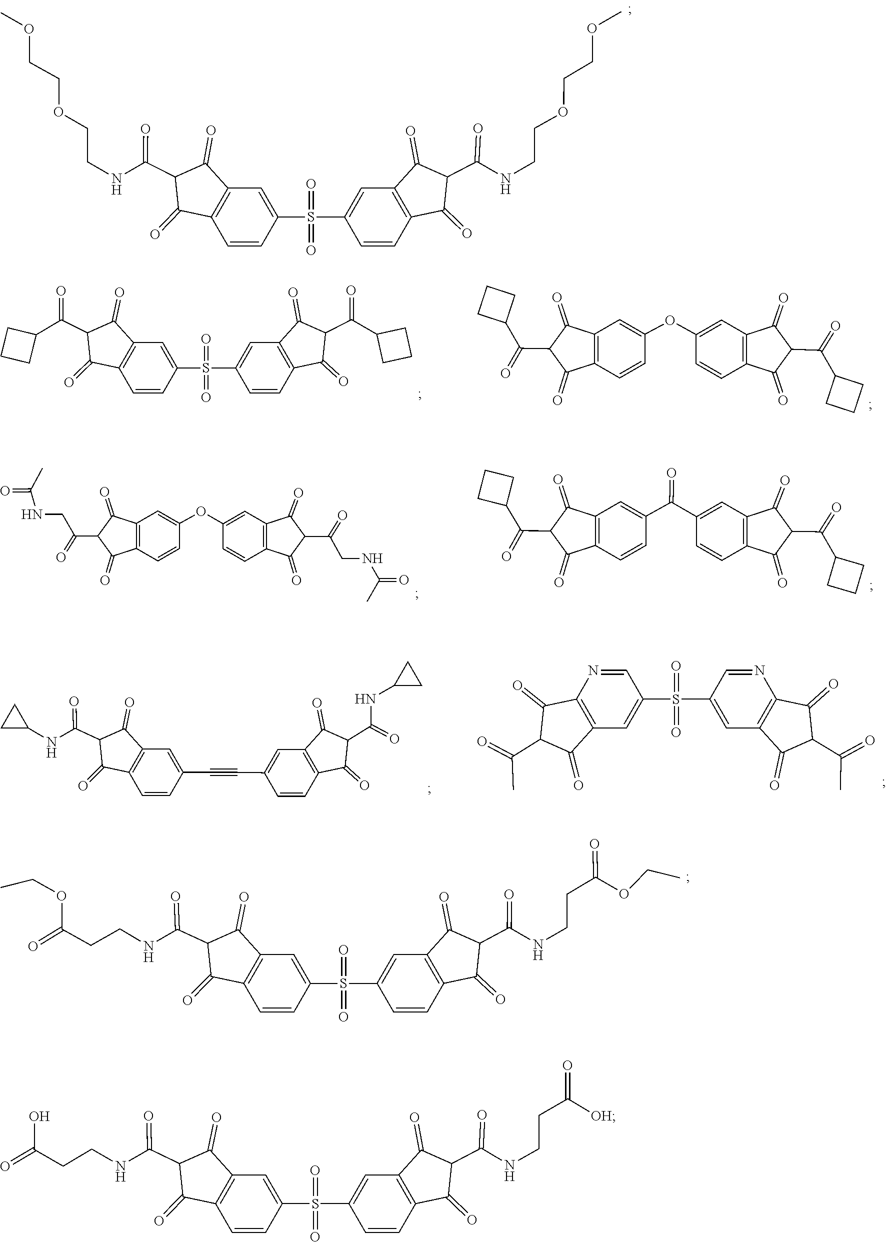

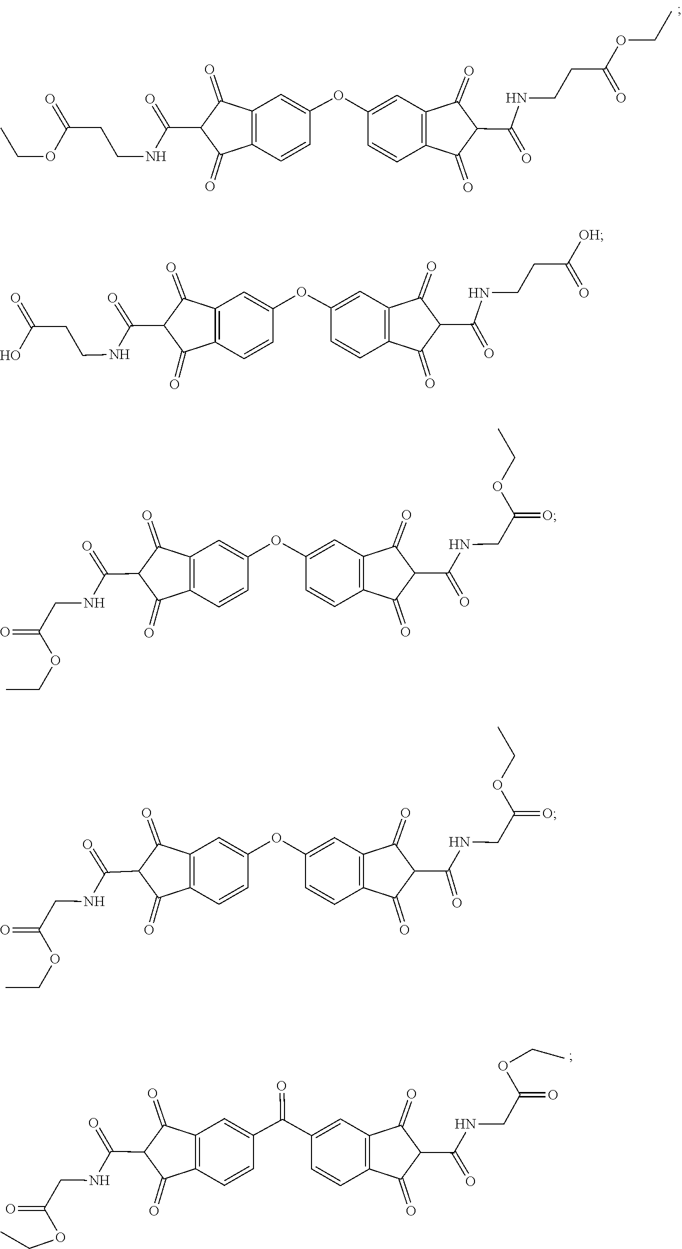

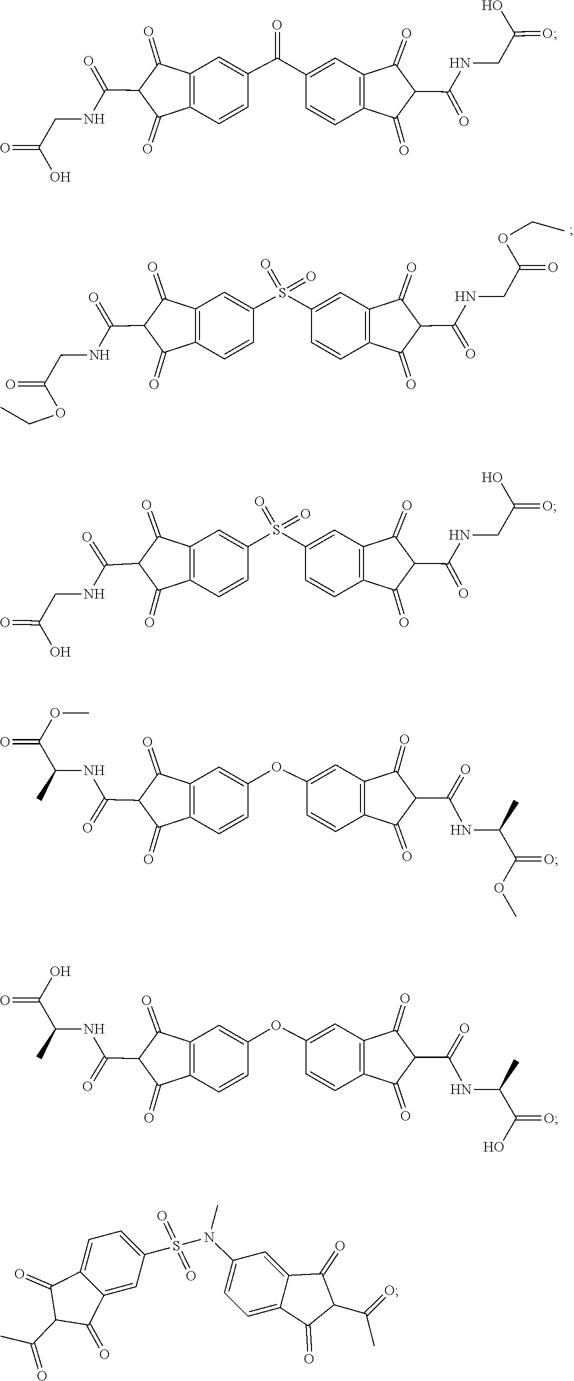

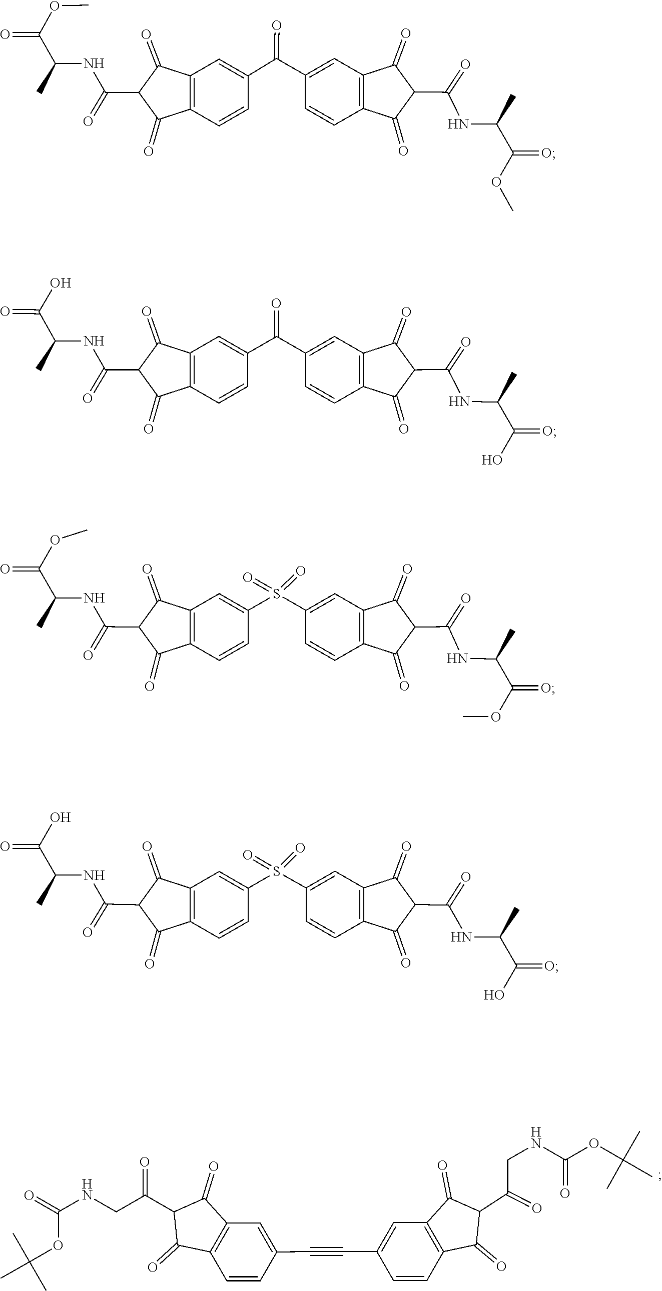 [SMILES: COC(=O)[C@H](C)NC(=O)C1C(=O)c2ccc(C(=O)c3ccc4c(c3)C(=O)C(C(=O)N[C@@H](C)C(=O)OC)C4=O)cc2C1=O ; C[C@H](NC(=O)C1C(=O)c2ccc(C(=O)c3ccc4c(c3)C(=O)C(C(=O)N[C@@H](C)C(=O)O)C4=O)cc2C1=O)C(=O)O ; COC(=O)[C@H](C)NC(=O)C1C(=O)c2ccc(S(=O)(=O)c3ccc4c(c3)C(=O)C(C(=O)N[C@@H](C)C(=O)OC)C4=O)cc2C1=O ; C[C@H](NC(=O)C1C(=O)c2ccc(S(=O)(=O)c3ccc4c(c3)C(=O)C(C(=O)N[C@@H](C)C(=O)O)C4=O)cc2C1=O)C(=O)O ; CC(C)(C)OC(=O)NCC(=O)C1C(=O)c2ccc(C#Cc3ccc4c(c3)C(=O)C(C(=O)CNC(=O)OC(C)(C)C)C4=O)cc2C1=O]

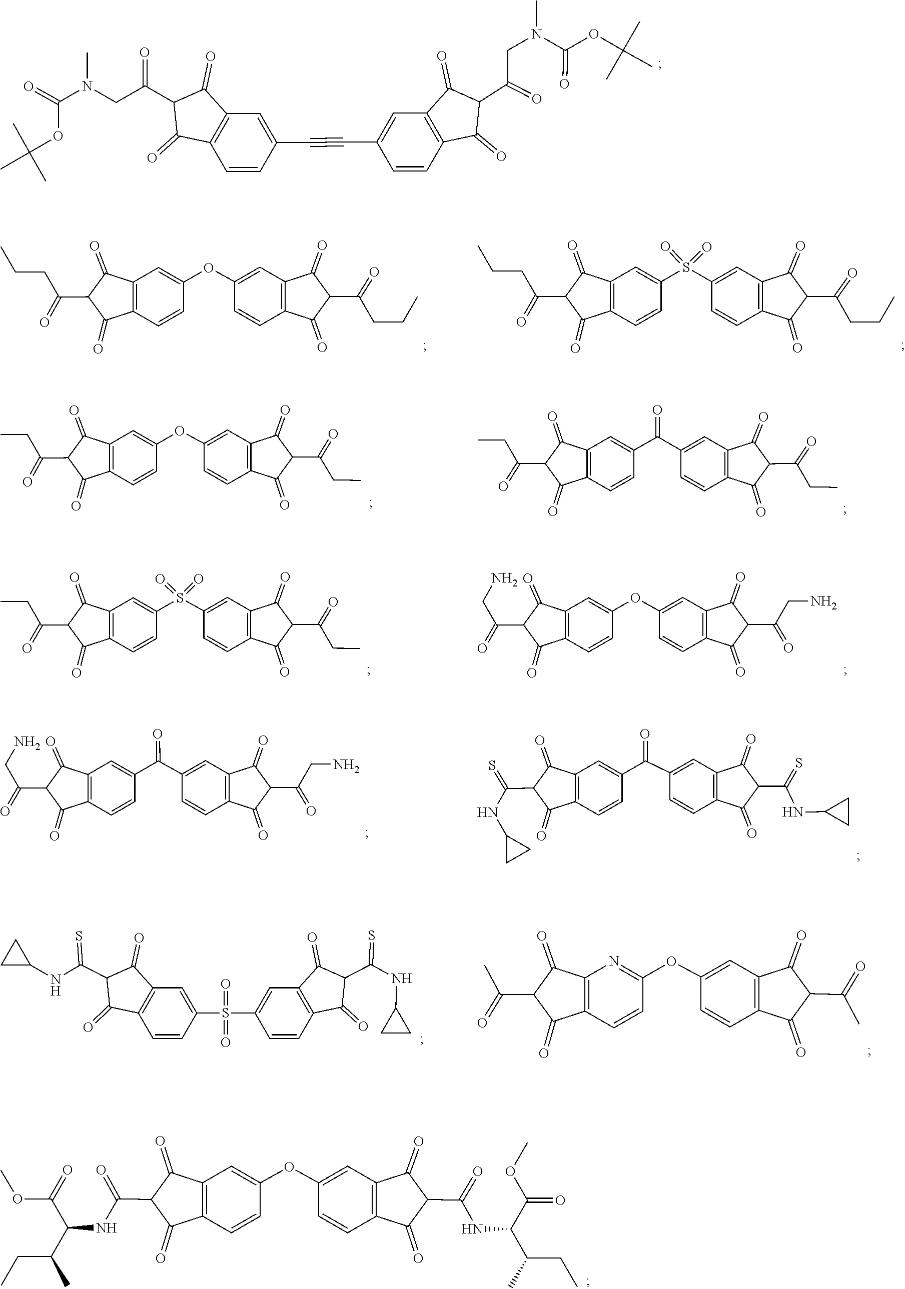

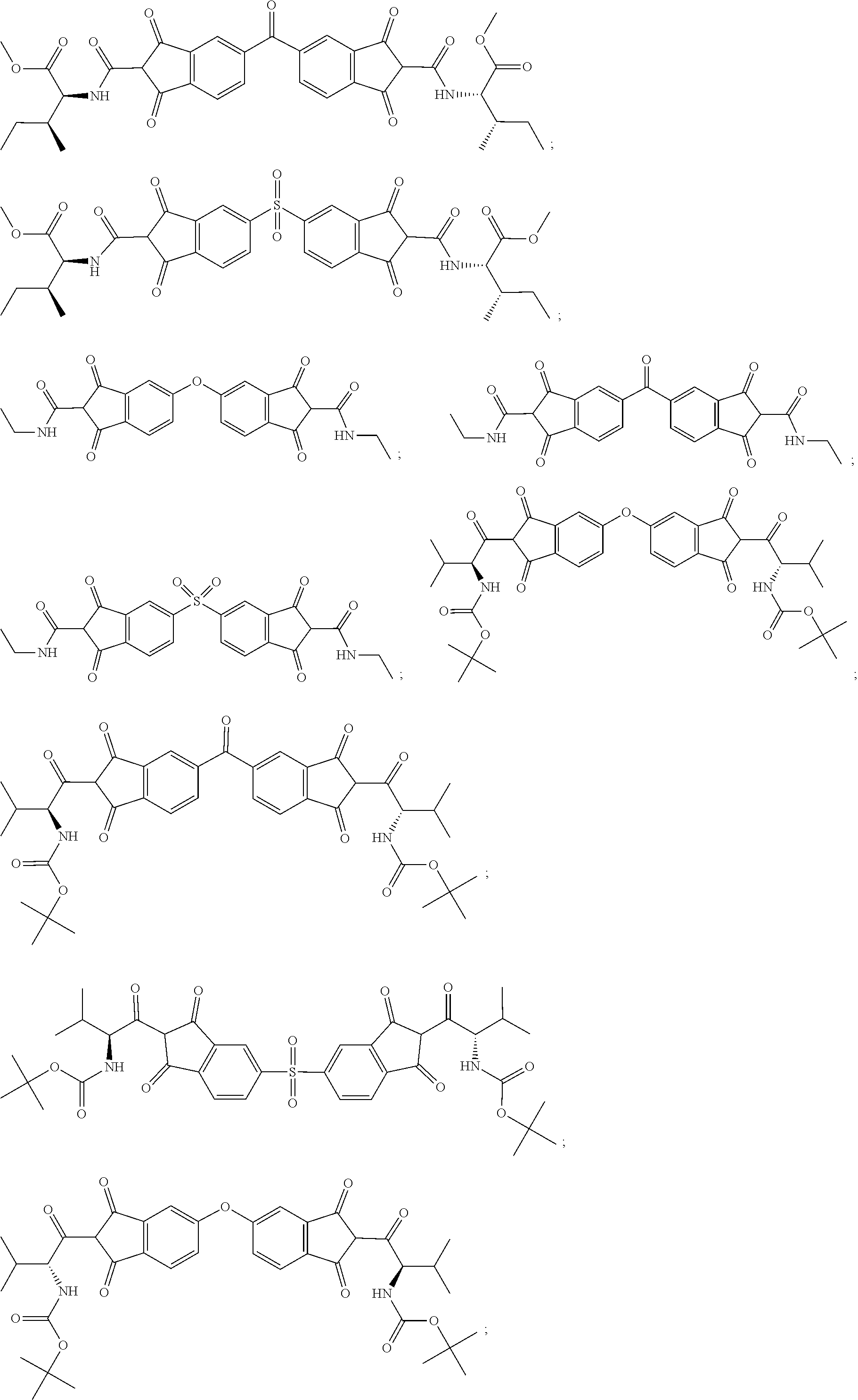

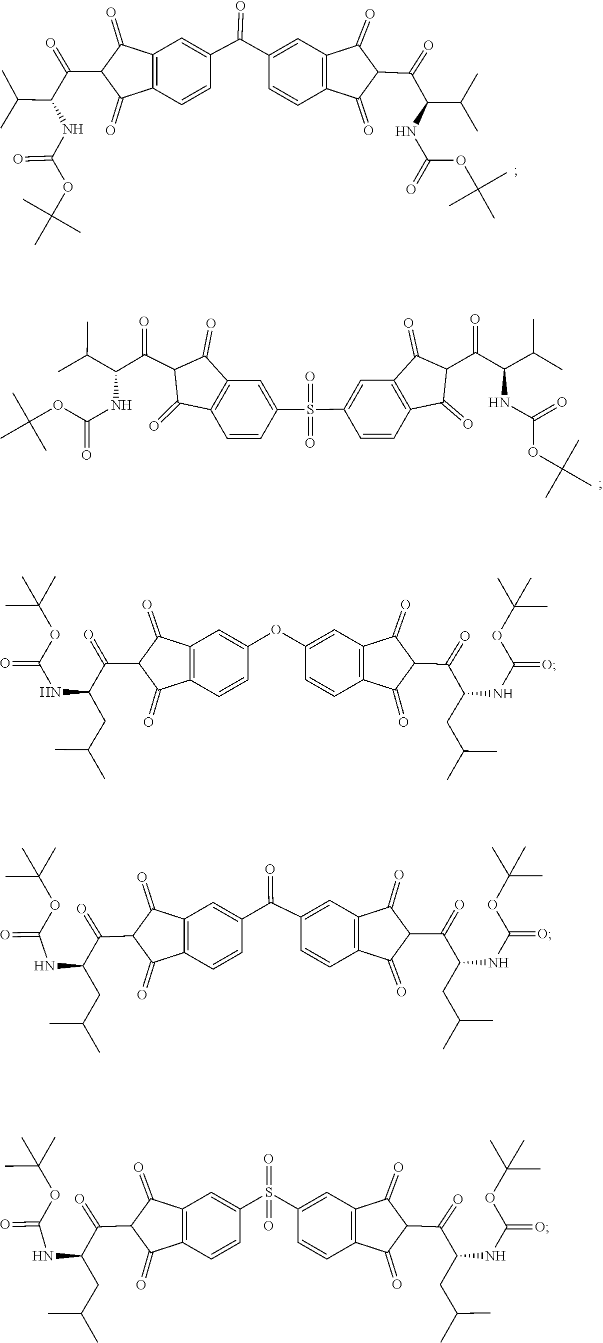

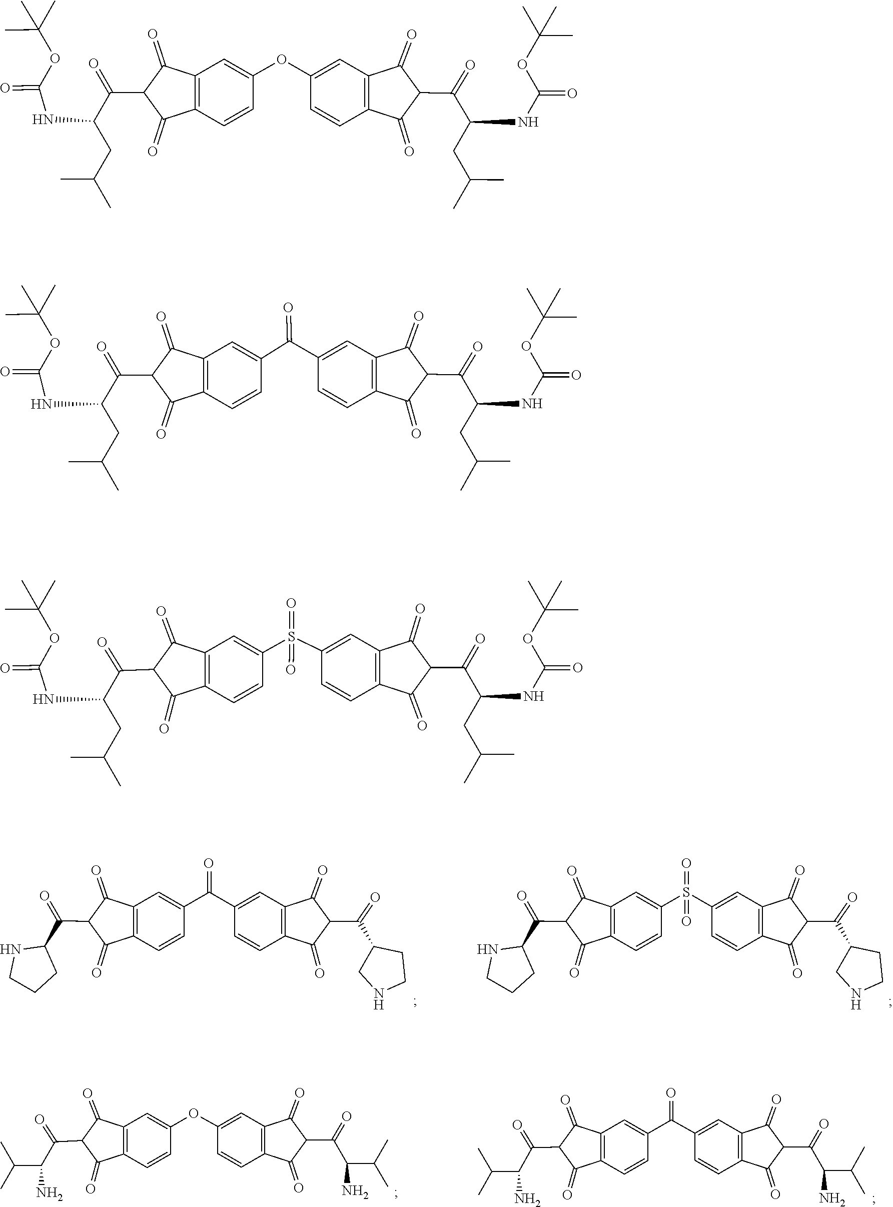

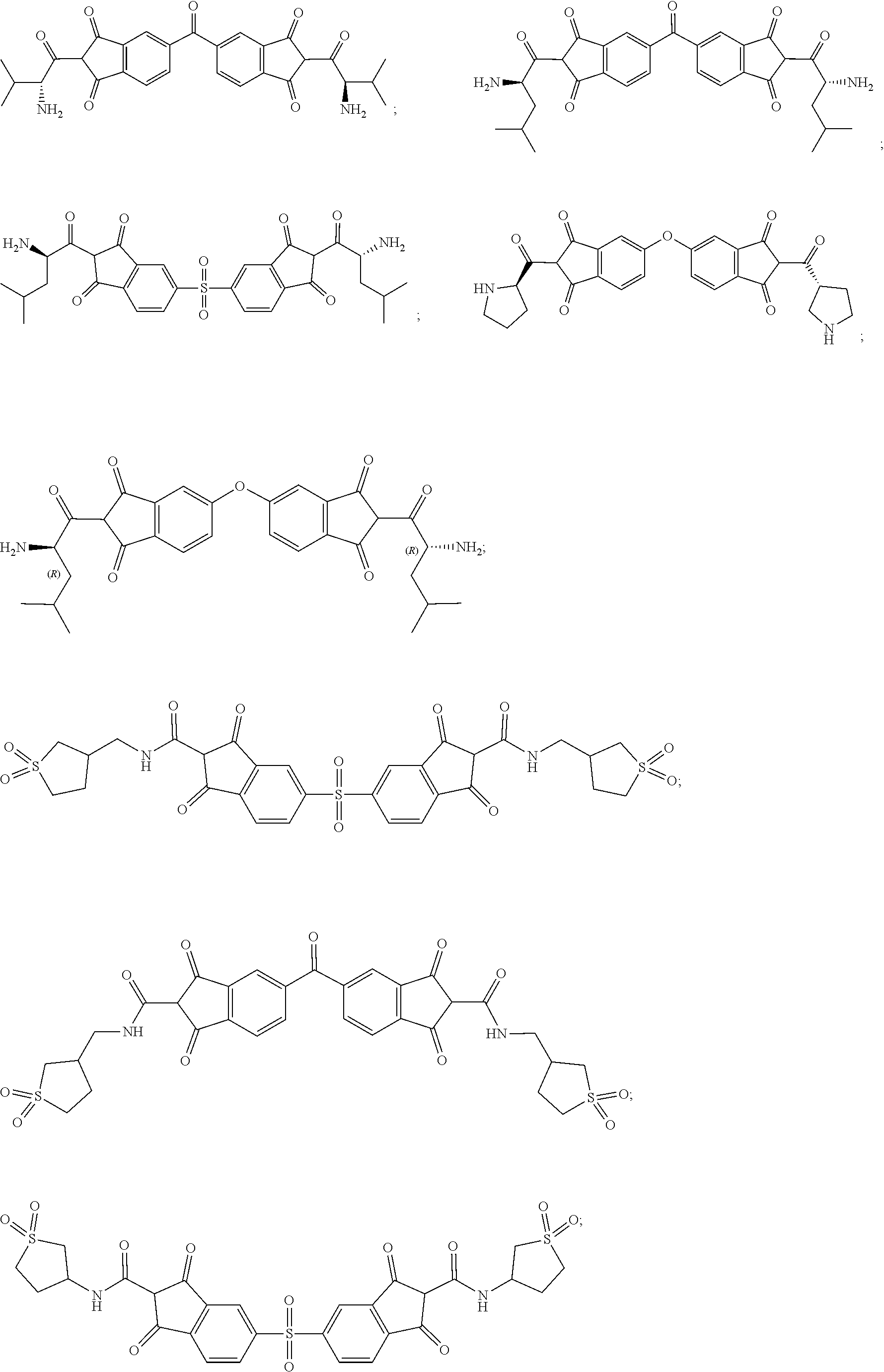

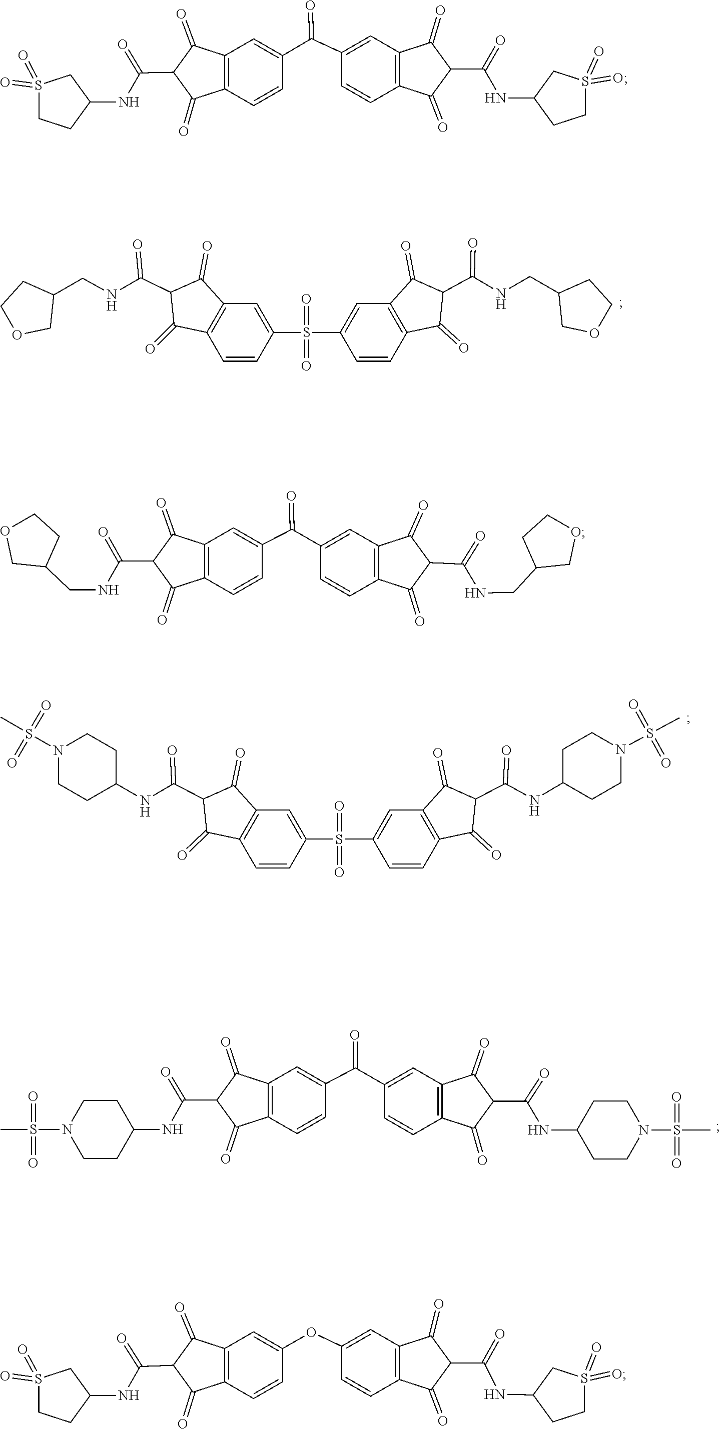

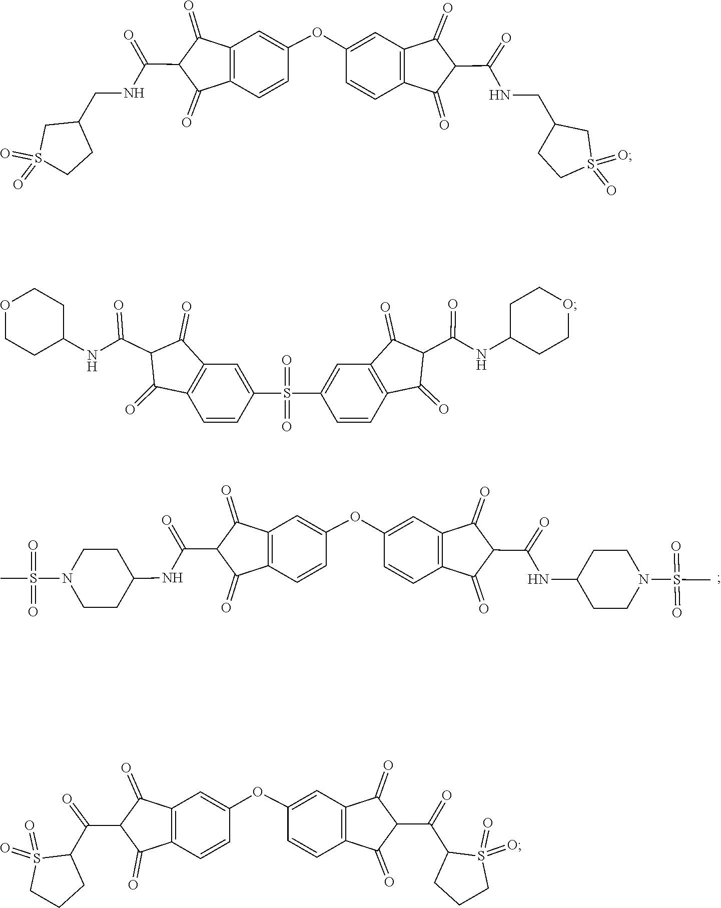

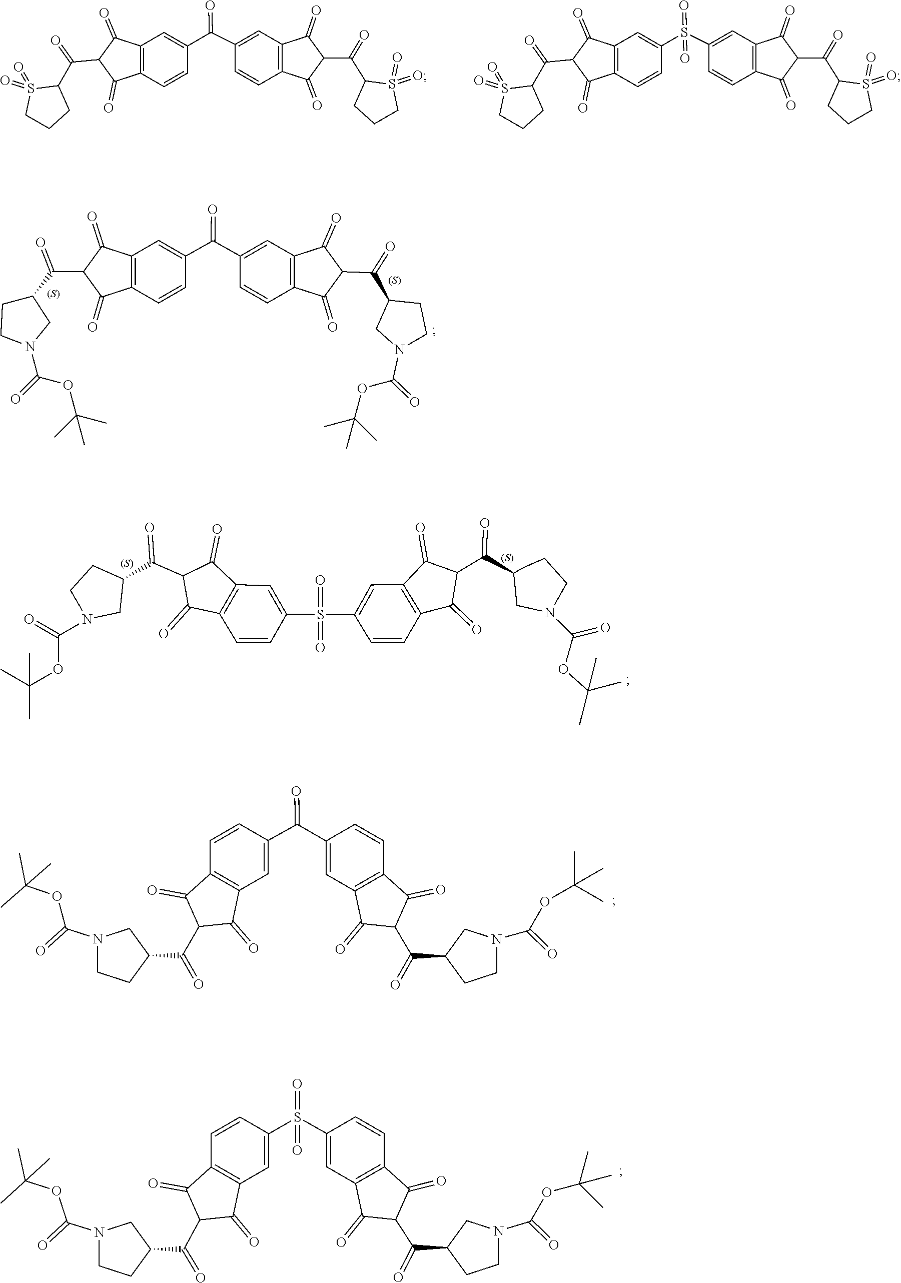

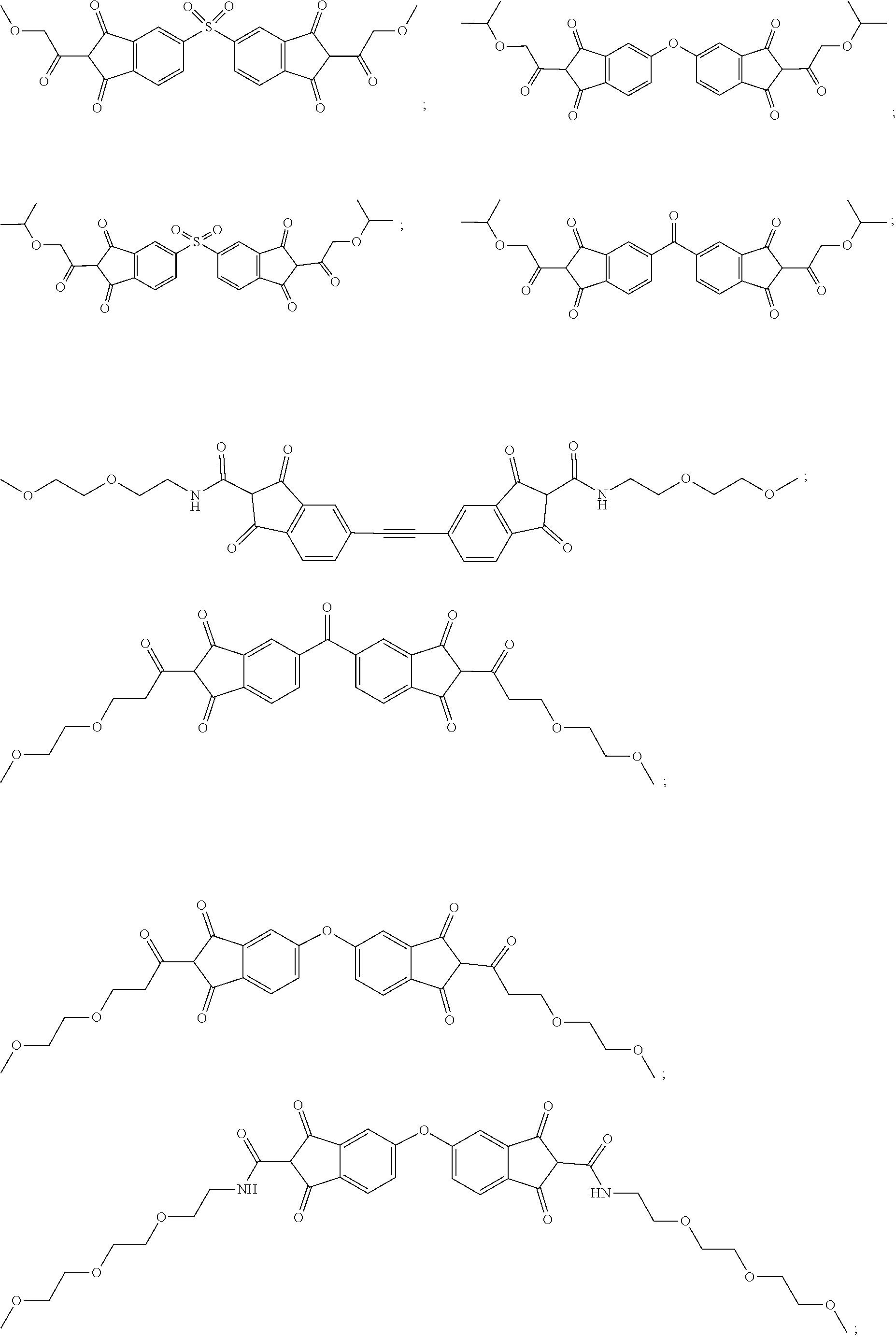

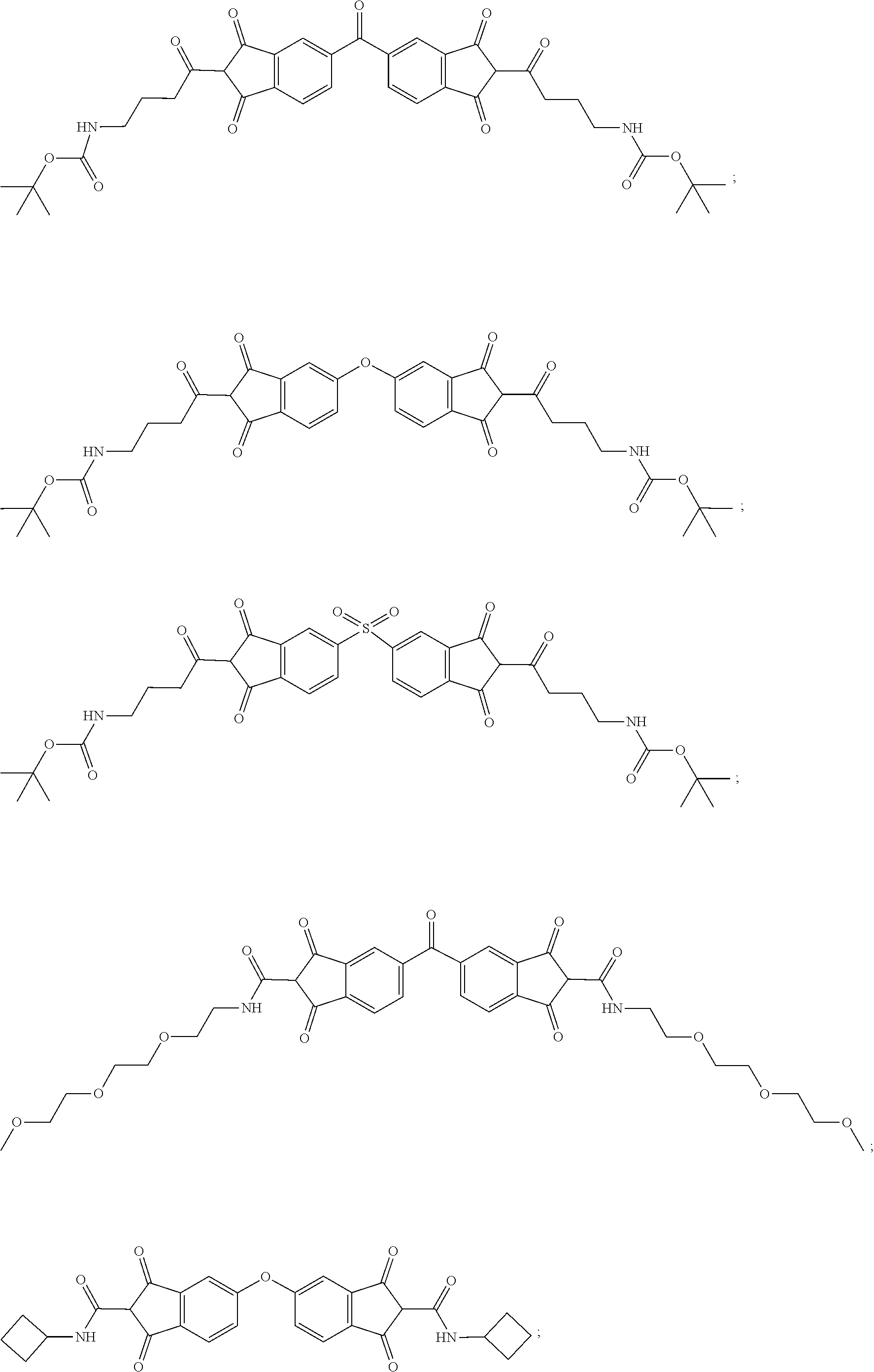

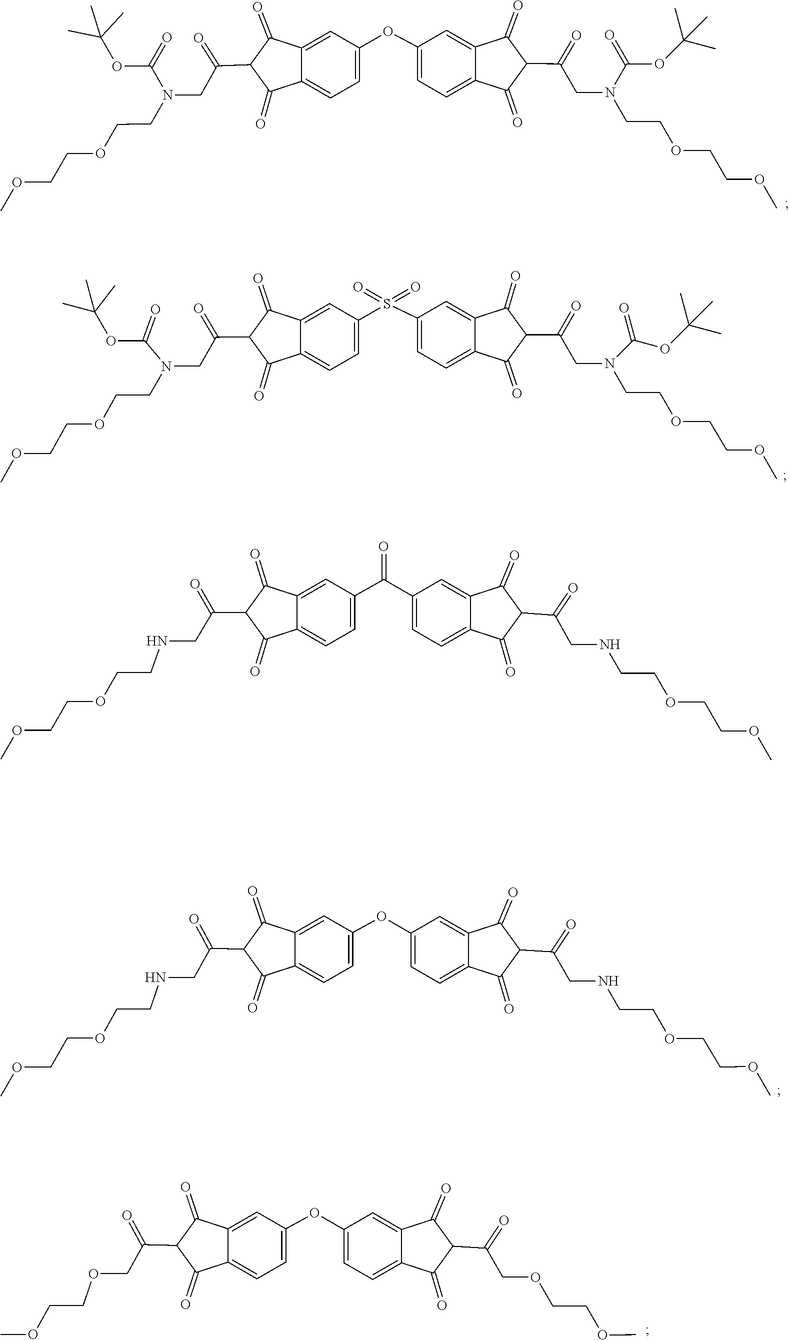

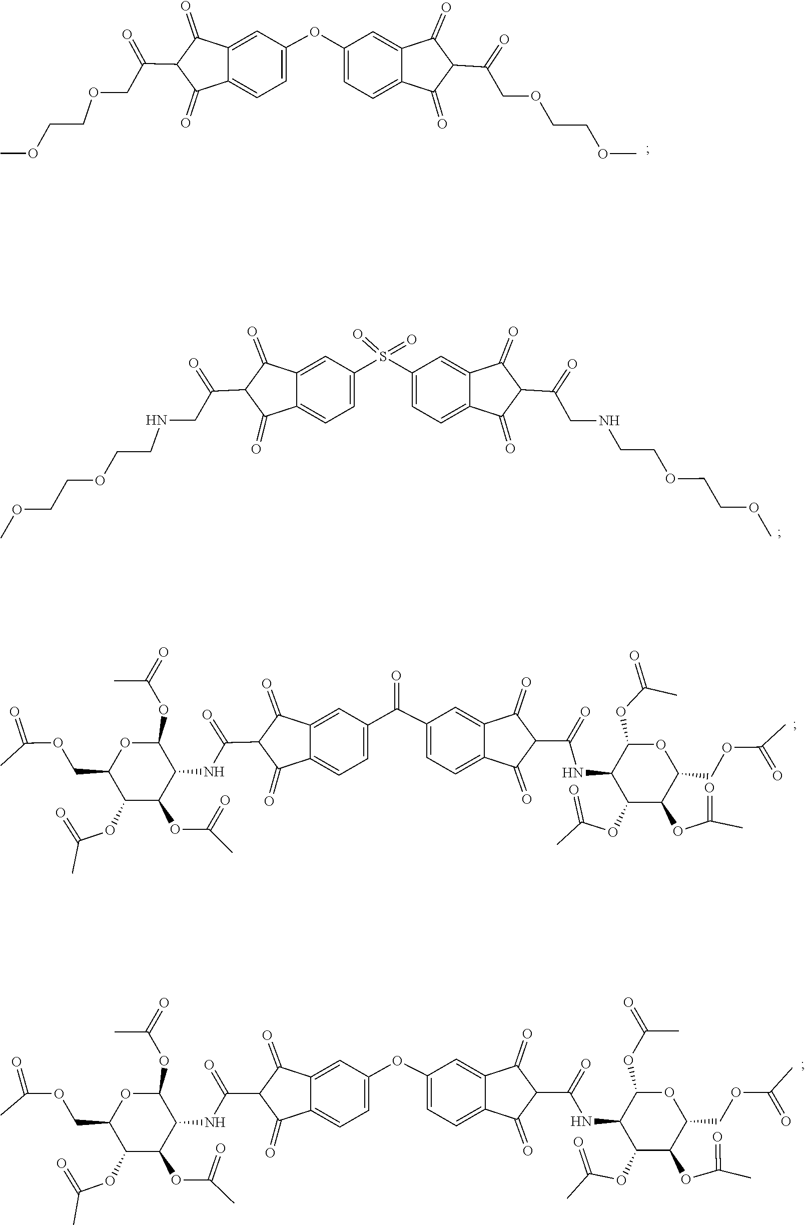

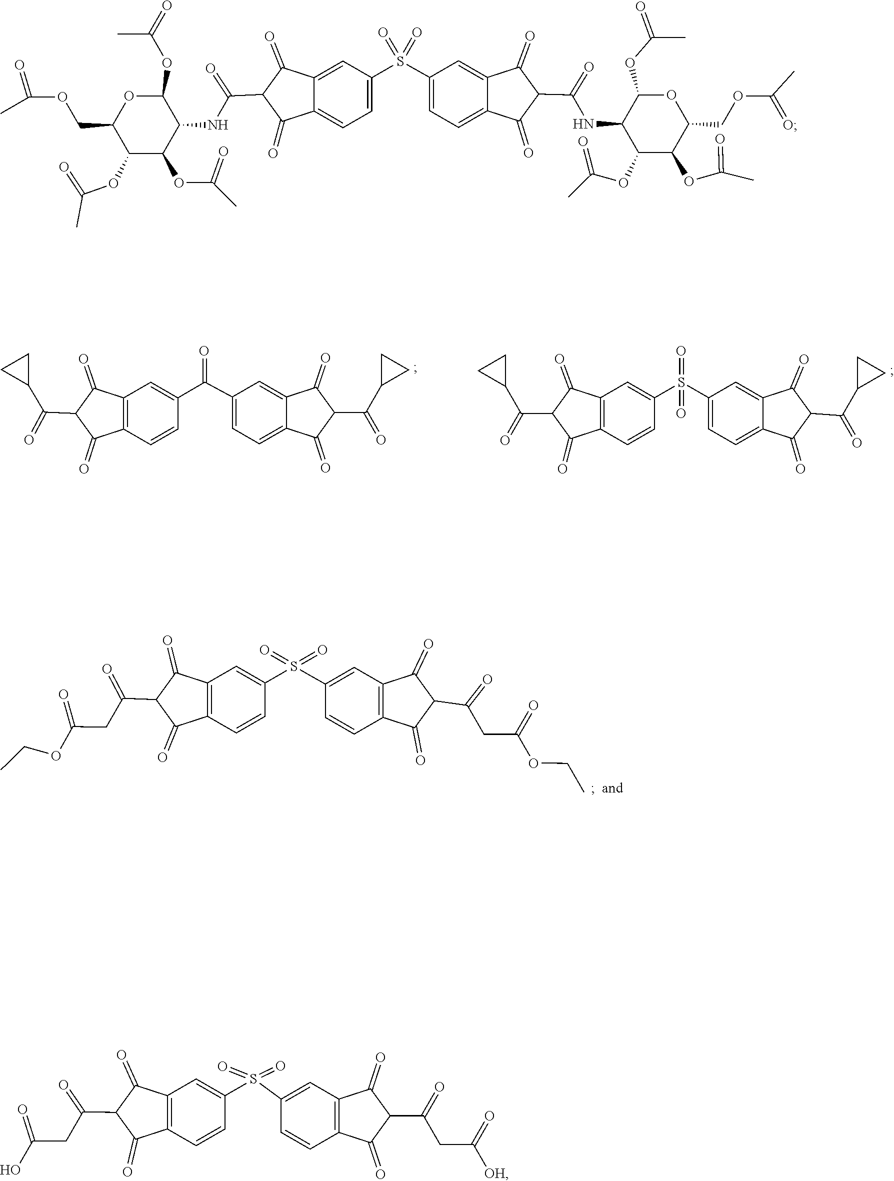

or pharmaceutically acceptable salt thereof.

In some embodiments, a salt of a compound of Formula (I) is formed between an acid and a basic group of the compound, such as an amino functional group, or a base and an acidic group of the compound, such as a carboxyl functional group. According to other embodiments, the compound is a pharmaceutically acceptable acid addition salt.

In some embodiments, acids commonly employed to form pharmaceutically acceptable salts of the compounds of Formula (I) include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-I,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, p-hydroxybutyrate, glycolate, maleate, tartrate, methanesu1fonate, propanesulfonate, naphthalene-1-sulfonate, naphthalene-2-sulfonate, mandelate and other salts. In some embodiments, pharmaceutically acceptable acid addition salts include those formed with mineral acids such as hydrochloric acid and hydrobromic acid, and especially those formed with organic acids such as maleic acid.

In some embodiments, bases commonly employed to form pharmaceutically acceptable salts of the compounds of Formula (I) include hydroxides of alkali metals, including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl)amine or tri-(2-hydroxyethyl)amine; N-methyl-D-glucamine; morpholine; thiomorpholine; piperidine; pyrrolidine; and amino acids such as arginine, lysine, and the like.

In some embodiments, the compounds of Formula (I), or pharmaceutically acceptable salts thereof, are substantially isolated.

### Methods of Making the Compounds

Compounds of Formula (I), including salts thereof, can be prepared using known organic synthesis techniques and can be synthesized according to any of numerous possible synthetic routes. For example, the compounds described herein may be prepared using methods and procedures similar to those of Examples 1-38, 65-196, and 209-221 herein. A person skilled in the art knows how to select and implement appropriate synthetic protocols, and appreciates that the processes described are not the exclusive means by which compounds provided herein may be synthesized, and that a broad repertoire of synthetic organic reactions is available to be potentially employed in synthesizing compounds provided herein.

In some embodiments, any one of the compounds of Formula (I) may be prepared according to the synthetic routes outlined in Schemes 1a and 1b, using methods and procedures similar to those of Examples 1-38.

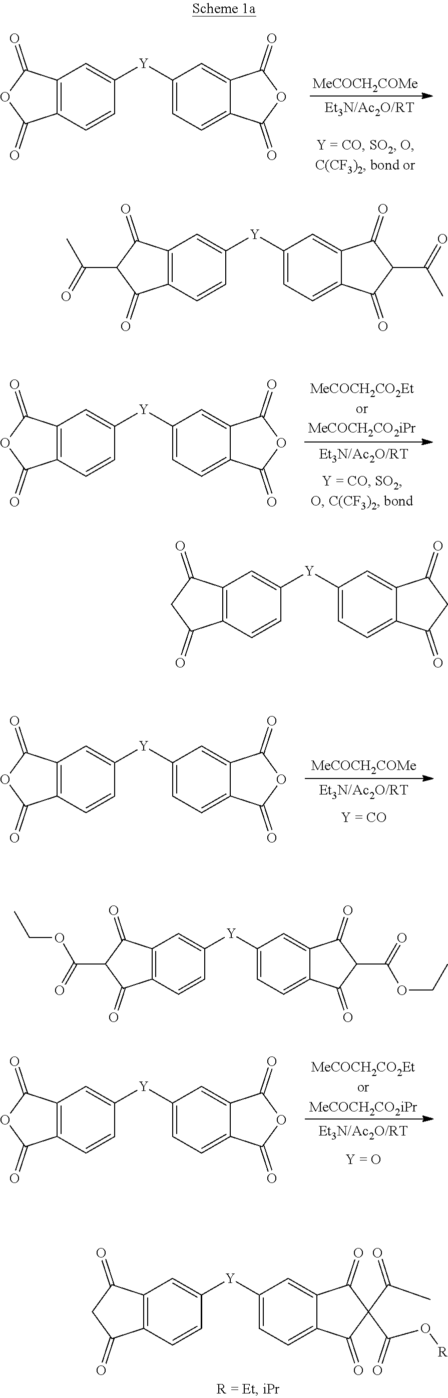

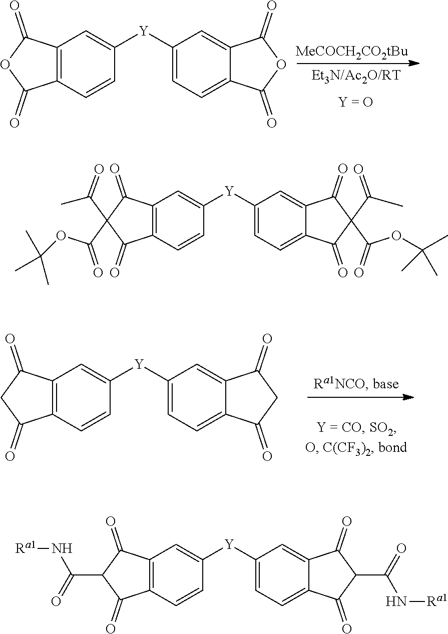

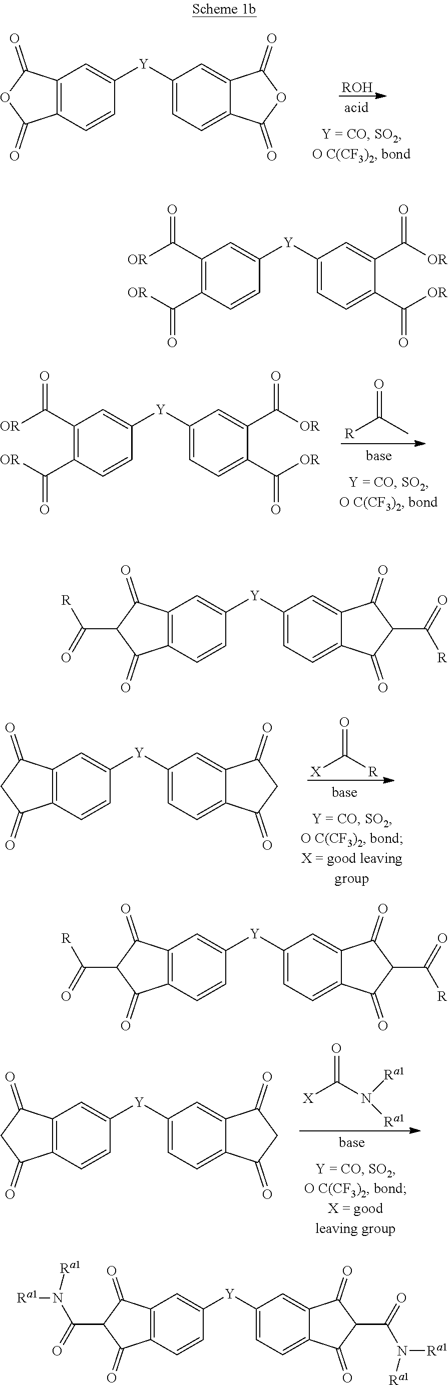

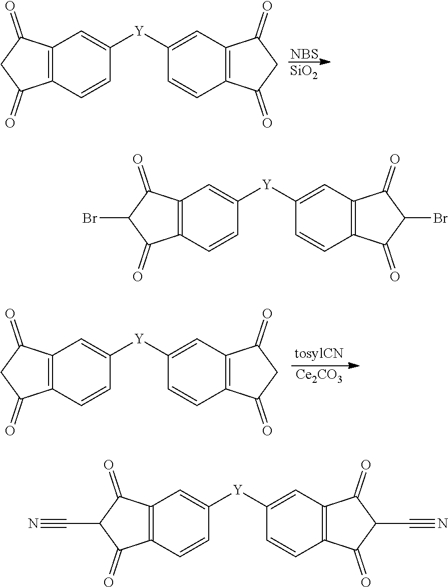

Referring to Scheme 1 b, the R— group may correspond to C₁₋₆ alkoxy, C₁₋₆ alkyl or an NH(R^{a1}) fragment, as in any one of the substituents R¹-R⁴ in a compound of Formula (I) described herein.

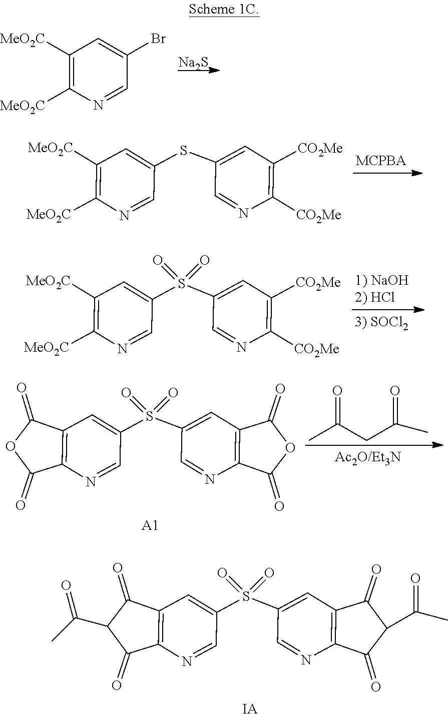

Referring to Scheme 1C, preparation of 6-acetyl-3-({6-acetyl-5,7-dioxo-5H,6H,7H-cyclopenta[b]pyridin-3-yl}sulfonyl)-5H,6H,7H-cyclopenta[b]pyridine-5,7-dione of Formula (I) described herein.

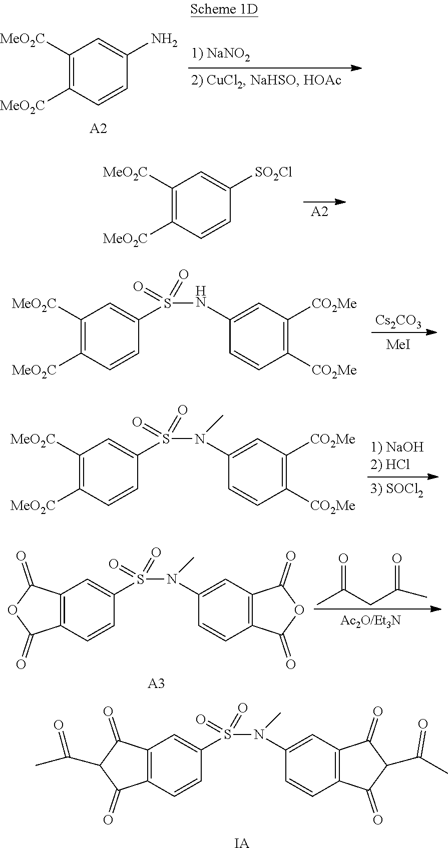

Referring to Scheme 1D, preparation of 2-acetyl-N-(2-acetyl-1,3-dioxo-2,3-dihydro-1H-inden-5-yl)-N-methyl-1,3-dioxo-2,3-dihydro-1H-indene-5-sulfonamide of Formula (I) described herein.

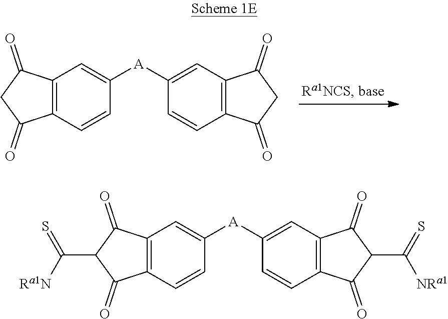

Refering to Scheme 1 E, an isothiocanate R^{a1}NCS may be used to prepare thioamide adducts of formula (I).

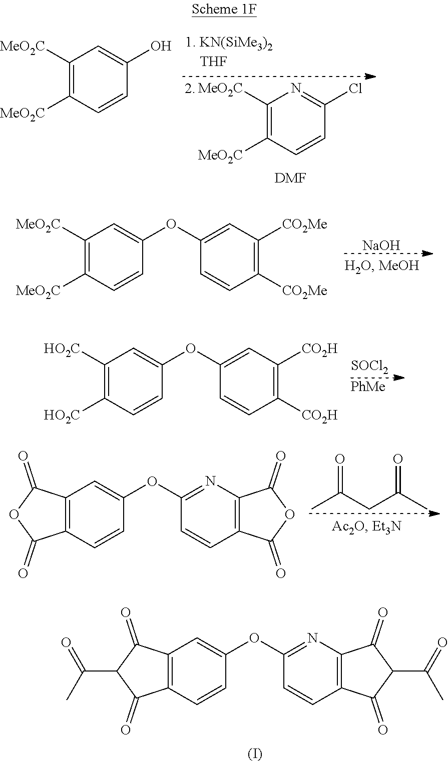

Compounds of formula (I) can be prepared from the methods employed in Scheme 1F.

Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II (Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

In some embodiments, the compounds of Formula (I) may be prepared according to the methods and procedures similar to those described in Larsen B. J. et al., Tetrahedron, 2018, 2762-2768, which is incorporated herein by reference in its entirety.

The reactions for preparing the compounds provided herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of the compounds provided herein can involve the protection and deprotection of various chemical groups. The need for protection and deprotection, and the selection of appropriate protecting groups, can be readily determined by one skilled in the art. The chemistry of protecting groups can be found, for example, in P. G. M. Wuts and T. W. Greene, Protective Groups in Organic Synthesis, 4^{th} Ed., Wiley & Sons, Inc., New York (2006).

### Methods of Use

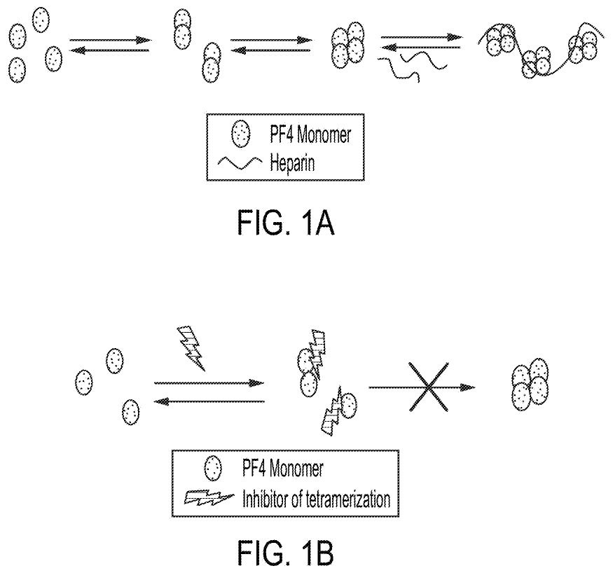

Disruption of the functionally-active PF4 tetrameric state to form functionally-inactive monomers and dimers also inhibits PF4 function. To form a ULC, PF4 must be in the tetrameric state. As schematically shown in FIG. 1, inhibition of formation of a PF4 tetramer (FIG. 1B) consequently inhibits the formation of the ULC with heparin.

Although PF4 crystallizes as a tetramer, in solution it exists in a dynamic equilibrium between monomeric, dimeric and tetrameric forms (see scheme in FIG. 1). As such, the relative abundance of the oligomeric form of PF4 can be influenced by shifting this equilibrium. Given that the salt bridges between Glu28 and Lys50 are predicted to stabilize the tetrameric form of PF4, Lys50 was mutated to glutamic acid in an attempt to disrupt tetramer formation. PF4 with this mutation (K50E) readily forms dimers, but not tetramers. Importantly, ULCs are not formed when heparin is incubated with K50E mutated PF4. In accordance with this observation, PF4 antagonists of the present disclosure (which are thought to bind near these residues) inhibit both tetramerization and ULC formation.

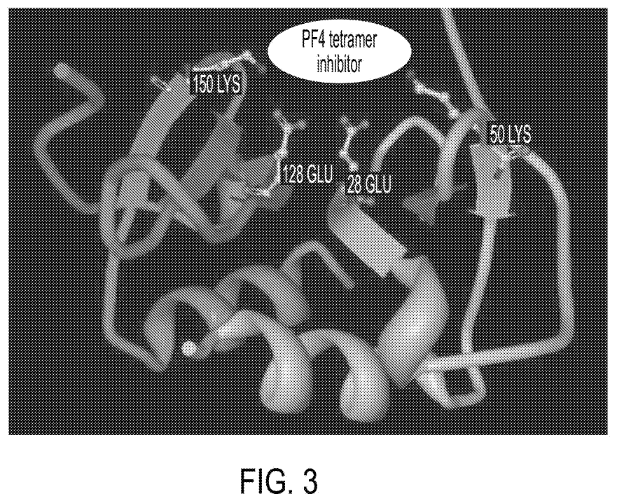

In some embodiments, the compounds described herein may be docked and scored on their ability to bind to the dimer interface pocket lined by monomer lysines and glutamine residues in the protein structure of PF4 (see, e.g., FIG. 3).

In some embodiments, the compound of Formula (I) prevent formation of and/or disrupt the PF4 tetramer. The prevention and/or disruption may occur in vitro, ex vivo or in vivo. For example, the prevention of disruption may occur in a subject (i.e., after administering the compound to the subject), e.g., in need thereof.

In some embodiments, the compound of Formula (I) may prevent formation and/or disrupt complexes formed between glycosaminoglycan (GAG) or other polyanion and a PF4 tetramer. In some embodiments, pathogenic complexes of GAG and PF4 are very large and are referred to as ultralarge complexes (ULC). GAG (e.g., heparin):PF4 complexes smaller than 600 kDa are typically referred to as small complexes (SC). In some embodiments, ULCs are 600 kDa or larger. In other embodiments, ULCs are 670 kDa or larger.

GAGs are long unbranched polysaccharides having a repeating disaccharide unit (a hexose (six-carbon sugar) or a hexuronic acid, linked to a hexosamine (six-carbon sugar containing nitrogen)). In some embodiments, the GAG is selected from among wild-type GAGs or synthetically produced GAGs. In some embodiments, the GAG is heparin, hyaluronan, hyaluronic acid, dermatan sulfate, keratan sulfate, or a chondroitin, or a salt thereof. In some embodiments, the GAG is heparin. In a further embodiment, the GAG is heparan sulfate.

PF4:heparin ULCs are more pathogenic than heparin:PF4 SCs. Heparin:PF4 ULCs are better recognized by HITT antibodies and lead to more platelet activation in the presence of these antibodies. Disruption of ULC represents a valid therapeutic target in the treatment of a disease mediated by the ULC, such as the HITT.

In some embodiments, the compounds described herein bind to PF4 monomers, PF4 trimers, PF4 dimers, and/or PF4 tetramers and inhibit the formation of the PF4 tetramers and/or inhibit a ULC formed with the PF4 tetramers. In some embodiments, the present compounds also disrupt a salt bridge in a PF4 tetramer and thereby disrupt or inhibit formation of the PF4 tetramer. In some embodiments, the compound binds to a specific site (e.g., specific amino acid) at the PF4 tetramer, trimer, dimer or monomer. In some embodiments, the compounds are capable of antagonizing an electrostatic attraction between the PF4 monomers, dimers, and trimers in the PF4 tetramer, and therefore successfully disrupt the salt bridge of the tetramer. For example, a functional group of the present compound of Formula (I) binds stronger to the PF4 monomer, dimer, or trimer, than they bind to one other.

The salt bridge between oligomers in the PF4 tetramer is formed via electrostatic interactions of a negatively charged amino acid, such as glutamic acid, of a first PF4 monomer or PF4 dimer, and a positively charged amino acid, such as lysine, of a second PF4 monomer or PF4 dimer. This salt bridge is typically formed by interaction of at least one Glu or at least one Lys on a first PF4 monomer or PF4 dimer, and at least one Lys or at least one Glu on a second PF4 monomer or PF4 dimer. In some embodiments, the salt bridge is formed via at least electrostatic interactions between Lys50 in a first PF4 monomer Glu28 in a second PF4 monomer. In other embodiments, the salt bridge is formed an electrostatic interaction between Glu128 or Lys350 of a first PF4 monomer or dimer, and Glu328 or Lys150 of a second PF4 monomer or dimer. In other embodiments, the salt bridge is formed via an electrostatic interactions between Glu228 or Lys450 of a first PF4 monomer or dimer and Glu428 or Lys250 of a second PF4 monomer or dimer. In some embodiments, the salt bridge is formed on a PF4 dimer-dimer interface. That is, the compound of the present disclosure disrupts the salt bridge on the PF4 dimer-dimer interface.

Typically, a PF4:heparin ULC is antigen that promotes production of an antibody that is specific to the complex. The anybody recognizes the complex composed of heparin and the PF4 tetramer, and forms a pathogenic ULC-antibody complex. By inhibiting formation and/or disruption PF4 tetramer, the compounds described herein inhibit binding of the pathogenic ULC-antibody complex to a FcγRIIa receptor on a surface of a platelet. This leads to inhibition of platelet activation, and results in decreased production of PF4 by the platelet. When the platelet is contacted with the compound in vitro, ex vivo, or in vivo (e.g., by administering the compound to the subject), the inhibition of binding of ULC-antibody complex to a FcγRIIa also leads to inhibition of platelet aggregation, increased high density lipoproteins, modulated (e.g., reduced) blood clotting or hemostasis, and corrected platelet imbalance in the subject (e.g., in need thereof). In some embodiments, the platelet imbalance results from heparin administration to the subject.

In one general aspect, the present application provides a method for diagnostic use or for use in determination of a dosage, or for pharmaceutical evaluation of a composition containing a compound of Formula (I) as described herein. Such methods include measuring a first level of PF4 tetramer in a first biological sample obtained from a subject and administering a first effective amount of the compound of Formula (I) that is required to decrease the first PF4 tetramer level. In other embodiments, methods for disrupting PF4 tetramers are provided and include measuring a first level of PF4 tetramer in a first biological sample obtained from a subject, administering a first effective amount of the compound of Formula (I) required to decrease the first PF4 tetramer level, and optionally administering a medication which disrupts PF4 tetramers. The medication which disrupts PF4 tetramers may be administered prior to, concurrent with, or subsequent to the PF4 antagonists discussed herein.

In other embodiments, methods for disrupting PF4 tetramers are provided and include measuring a first level of PF4 tetramer in a first biological sample obtained from a subject, administering a first effective amount of the compound of Formula (I) required to decrease the first PF4 tetramer level, measuring a second level of PF4 tetramer in a second biological sample obtained from the subject, and administering a second effective amount of the compound of Formula (I) required to decrease the second PF4 tetramer level. Multiple samples can be obtained from the subject at any interval required, for example, to prevent or treat the medical condition.

In a further embodiment, methods for disrupting ULCs containing PF4 tetramers and heparin are provided. These methods include measuring a first level of ULCs in a first biological sample obtained from a subject and administering a first effective amount of the compound of Formula (I) required to decrease the first ULC tetramer level.

In yet other embodiments, methods for disrupting ULCs containing PF4 tetramers and heparin include measuring a first level of ULCs in a first biological sample obtained from a subject, administering a first effective amount of the compound of Formula (I) required to decrease the first ULC level, measuring a second level of ULCs in a second biological sample obtained from the subject, and administering a second effective amount of the compound of Formula (I) required to decrease the second ULC level.

In still a further embodiment, methods for preventing the formation of PF4 tetramers include measuring a first level of PF4 tetramer in a first biological sample obtained from a subject, administering a first effective amount of the compound of Formula (I) required to prevent formation of the PF4 tetramer, measuring a second level of PF4 tetramer in a second biological sample obtained from the subject, and administering a second effective amount of the compound of Formula (I) required to prevent formation of PF4 tetramer.

Also provided are methods for using the antagonistic compounds described herein for determining a subject's sensitivity to side effects or secondary medical conditions related to heparin administration. Further provided are methods for determining the likelihood of a subject to acquire a medical condition related to the formation of PF4 tetramers or ULCs containing PF4 tetramers and heparin. In some embodiments, these screening methods are useful in monitoring e.g., cancer patients. In other embodiments, these screening methods are useful in determining the likelihood of cancer patients being administered heparin in developing HIT or HITT. According to this method, biological samples are obtained from subjects and the level of PF4 tetramer and/or PF4 tetramer:GAG (heparin) ULC measured. The screening may be conducted using techniques commonly known and used in the art. Comparison of the levels of PF4 tetramer and/or PF4 tetramer:heparin ULC to a control level and/or negative control provides evidence that the patient may be treated using one or more of the antagonistic compounds described herein. In some embodiments, if the subject's PF4 tetramer and/or PF4 tetramer:heparin ULC level is higher than the PF4 tetramer and/or PF4 tetramer:heparin ULC level of a healthy subject, then antagonistic compound administration may be contemplated.

In some embodiments, the present application provides a method of treating a disease or condition characterized by PF4 tetramerization (e.g., a disease or medical condition related directly or indirectly to the formation of PF4 tetramers). The disease or medical condition may also be caused by the formation of PF4 tetramers. In some embodiments, the subject or patient has elevated levels of PF4 tetramer. In yet a further embodiment, the subject or patient has elevated levels of PF4 tetramer:GAG ULCs.

Suitable examples of a disease or condition include, but are not limited to, heparin-induced thrombocytopenia (HIT), heparin-induced thrombocytopenia and thrombosis (HIT), thrombotic complication of HITT, atherosclerosis or atherosclerotic vascular disease, platelet imbalance or insufficiency, antiphospholipid syndrome, inflammation or inflammatory disease, vaccine-induced immune throbotic thrombocytopenia (VITT), or clotting or hemostasis disorders.

In some embodiment, the disease or medical condition is heparin-induced thrombocytopenia (HIT). HIT results from the development of thrombocytopenia (low platelet count), for example, due to the administration of an anticoagulant (e.g., heparin or warfarin). In other embodiments, the disease or medical condition is heparin-induced thrombocytopenia and/with thrombosis (HITT). HITT results when HIT precedes thrombosis (abnormal antibodies and abnormal blood clots form inside a blood vessel). In some embodiments, the thrombosis is characterized by lower than normal thrombin-antithrombin complex (TAT) level.

In other embodiments, the disease or medical condition is vaccine-induced immune thrombotic thrombocytopenia (VITT) also known as thrombosis with thrombocytopenia syndrome. In some embodiments, the VITT is caused by an adenovirus-based vaccine. In some embodiments, the vaccine is a adenovirus-based SARS-CoV-2 vaccine.

Treatment of atherosclerotic vascular disease typically involves anti-platelet therapy (e.g, aspirin and Plavix® that is not tolerated in all patients). For the treatment of thrombocytopenia, TPO analogs and mimetics (MPL agonists which activate MPL—the TPO receptor) may be used, but these drugs have significant side effects and compliance issues. Finally, some lipid lowering therapies are able to increase HDL, but the ability of available drugs to do so is limited. In other embodimenta, the disease that may be successfully treated by the compound of Formula (I) is antiphospholipid syndrome.

In a further embodiment, atherosclerosis resulting from the formation of a PF4 tetramer may be treated using a compound of Formula (I) described herein. In still other embodiments, the disease or medical condition is a platelet imbalance. The treatment method thereby includes correcting this platelet imbalance or preventing a platelet imbalance. In one example, platelet levels are increased by stimulating platelet production. In another example, a decrease in platelet production is prevented. In a further example, the platelet imbalance (e.g., low levels of platelets) results from the formation of a PF4 tetramer. In yet another example, the platelet imbalance, i.e., low levels of platelets, results from heparin administration to a subject.

The compounds of Formula (I) discussed herein may also be an alternative therapy utilized to treat diseases related to thrombopoietin (TPO). The compounds may also be contemplated for use in preventing or treating inflammation which results from the formation of PF4 tetramers. The inflammation may be the caused by any number of factors. In some embodiments, the inflammation is acute or chronic. In other embodiments, the inflammation is localized or systemic. The inflammation may be the result of a variety of factors and/or conditions. The compounds of Formula (I) may also be useful in therapies for subjects having atherosclerotic vascular disease in which the patient is intolerant to the conventional treatments (e.g., statins). In some embodiments, an inflammatory disease is chronic inflammatory demyelinating polyneuropathy, inflammatory myopathy, inflammatory bowel diseases (IBDs), Crohn disease (CD), ulcerative colitis (UC), chronic inflammatory condition with polygenic susceptibility, inflammation of the uvea (e.g., anterior uveitis, e.g., iridocyclitis or iritis; intermediate uveitis (also known as pars planitis); posterior uveitis; or chorioretinitis, e.g., pan-uveitis).

The compounds described herein may further be useful for modulating clotting or hemostasis. In one example, the compounds may be useful in patients that are intolerant to conventional therapies. Alternatively, the compounds of the present application may be synergistic with the conventional therapies.

The compounds of Formula (I) are also useful in increasing high density lipoproteins (HDL) in a subject. Alternatively, the PF4 antagonists of the present application are useful in preventing a decrease of HDLs.

In some embodiments, a PF4-associated disease or condition in a subject may occur simultaneously with another disease or medical condition. In one example, a decrease in platelet production may develop in a patient diagnosed with cancer. In some embodiments, the cancer is selected from the group selected from sarcoma, angiosarcoma, fibrosarcoma, rhabdomyosarcoma, liposarcoma, myxoma, rhabdomyoma, fibroma, lipoma, teratoma, lung cancer, breast cancer, bronchogenic carcinoma squamous cell, undifferentiated small cell, undifferentiated large cell, adenocarcinoma, alveolar bronchiolar carcinoma, bronchial adenoma, sarcoma, lymphoma, chondromatous hamartoma, mesothelioma, gastrointestinal cancer, cancer of the esophagus, squamous cell carcinoma, adenocarcinoma, leiomyosarcoma, lymphoma, cancer of the stomach, carcinoma, lymphoma, leiomyosarcoma, cancer of the pancreas, ductal adenocarcinoma, insulinoma, glucagonoma, gastrinoma, carcinoid tumor, vipoma, cancer of the small bowel, adenocarcinoma, lymphoma, carcinoid tumors, Kaposi's sarcoma, leiomyoma, hemangioma, lipoma, neurofibroma, fibroma, cancer of the large bowel or colon, tubular adenoma, villous adenoma, hamartoma, leiomyoma, genitourinary tract cancer, cancer of the kidney, adenocarcinoma, Wilm's tumor (nephroblastoma), lymphoma, leukemia, cancer of the bladder, cancer of the urethra, squamous cell carcinoma, transitional cell carcinoma, cancer of the prostate, cancer of the testis, seminoma, teratoma, embryonal carcinoma, teratocarcinoma, choriocarcinoma, sarcoma, interstitial cell carcinoma, fibroma, fibroadenoma, adenomatoid tumors, lipoma, liver cancer, hepatoma hepatocellular carcinoma, cholangiocarcinoma, hepatoblastoma, angiosarcoma, hepatocellular adenoma, hemangioma, bone cancer, osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor, chordoma, osteochrondroma (osteocartilaginous exostoses), benign chondroma, chondroblastoma, chondromyxofibroma, osteoid osteoma giant cell tumor, nervous system cancer, cancer of the skull, osteoma, hemangioma, granuloma, xanthoma, osteitis deformans, cancer of the meninges meningioma, meningiosarcoma, gliomatosis, cancer of the brain, astrocytoma, medulloblastoma, glioma, ependymoma, germinoma (pinealoma), glioblastoma multiforme, oligodendroglioma, schwannoma, retinoblastoma, congenital tumors, cancer of the spinal cord, neurofibroma, meningioma, glioma, sarcoma, gynecological cancer, cancer of the uterus, endometrial carcinoma, cancer of the cervix, cervical carcinoma, pre tumor cervical dysplasia, cancer of the ovaries, ovarian carcinoma, serous cystadenocarcinoma, mucinous cystadenocarcinoma, unclassified carcinoma, granulosa-theca cell tumor, Sertoli Leydig cell tumor, dysgerminoma, malignant teratoma, cancer of the vulva, squamous cell carcinoma, intraepithelial carcinoma, adenocarcinoma, fibrosarcoma, melanoma, cancer of the vagina, clear cell carcinoma, squamous cell carcinoma, botryoid sarcoma, embryonal rhabdomyosarcoma, cancer of the fallopian tubes, hematologic cancer, cancer of the blood, acute myeloid leukemia (AML), chronic myeloid leukemia (CML), acute lymphoblastic leukemia (ALL), chronic lymphoblastic leukemia, chronic lymphocytic leukemia, myeloproliferative diseases, multiple myeloma, myelodysplastic syndrome, Hodgkin's lymphoma, non-Hodgkin's lymphoma (malignant lymphoma), Waldenstrom's macroglobulinemia, skin cancer, malignant melanoma, basal cell carcinoma, squamous cell carcinoma, Kaposi's sarcoma, moles dysplastic nevi, lipoma, angioma, dermatofibroma, keloids, adrenal gland cancer, and neuroblastoma.

In some embodiments, the cancer patient having a PF4-associated disease or condition is undergoing a chemotherapy. In one example, the PF4-associated disease or disorder is the result of the chemotherapy treatment. Suitable examples of chemotherapeutic agents include paclitaxel, docetaxel, daunorubicin, cis-platin, carboplatin, and others. In some embodiments, the present application provides a method of treating cancer in a subject (e.g., any one of cancers described herein), the method comprising administering to the subject a therapeutically effective amount of a compound of Formula (I), or a pharmaceutically acceptable salt thereof.

### Combinations

In another general aspect, the compound of Formula (I) as described herein may be administered to the subject in combination with an additional therapeutic agent. In one example, the additional therapeutic agent may disrupt PF4 tetramers and/or ULCs. In some embodiments, the additional agent works synergistically with the PF4 antagonist of the present application. Suitable examples of such agents include cyclic peptides which inhibit the interaction of PF4 with CCL5 (CKEY2), carbohydrates such as desulfated heparin (ODSH), or a combination thereof. The PF4 tetramer disruption agents may be combined with the PF4 antagonist compounds of the present application either in a pharmaceutical composition as described herein, and/or kits and methods for using the same.

In some embodiments, an additional therapeutic agent is an anticoagulant (e.g., rivaroxaban, dabigatran, apixaban, edoxaban, warfarin, fondaparinux, idraparinux, acenocoumarol, phenprocoumon, atromentin, or phenindione). In some embodiments, additional therapeutic agent is heparin. Other suitable examples of additional therapeutic agents include an anti-HER2 agent (e.g., trastuzumab, pertuzumab, lapatinib), a pain relief agent (e.g., a nonsteroidal anti-inflammatory drug such as celecoxib or rofecoxib), an antinausea agent, a cardioprotective drug (e.g., dexrazoxane, ACE-inhibitors, diuretics, cardiac glycosides), a cholesterol lowering drug, a revascularization drug, a beta-blocker (e.g., acebutolol, atenolol, bisoprolol, metoprolol, nadolol, nebivolol, or propranolol), an angiotensin receptor blocker (also called ARBs or angiotensin II inhibitors) (e.g., azilsartan, candesartan, eprosartan, irbesartan, losartan, olmesartan, telmisartan, or valsartan), and an anticancer agent (e.g., paclitaxel, docetaxel, daunorubicin, cis-platin, carboplatin, taxol, 5-fluorouracil, oxaliplatin/5 FU, abiraterone, or procarbazine).

In some embodiments, the compound of Formula (I) and the additional therapeutic agent may be administered to the subject simultaneously (e.g., in the same dosage form or in separate dosage forms), or consecutively (e.g., heparin may be administered before or the compound of Formula (I)). The dosages and routes of administration are well within the judgement of the treating physician.

### Kits

The present application also includes pharmaceutical kits useful, for example, in the treatment of disorders, diseases and conditions referred to herein, which include one or more containers containing a pharmaceutical composition comprising a therapeutically effective amount of a compound of the present disclosure. Such kits can further include, if desired, one or more of various conventional pharmaceutical kit components, such as, for example, containers with one or more pharmaceutically acceptable carriers, additional containers with additional therapeutic agents, diagnostic reagents, etc. Instructions, either as inserts or as labels, indicating quantities of the components to be administered, guidelines for administration, and/or guidelines for mixing the components, can also be included in the kit.

Optionally, the kit may further contain instructions for monitoring blood level of the administered compound, and materials for performing such assays including, e.g., reagents, well plates, containers, markers or labels, and the like. Such kits are readily packaged in a manner suitable for treatment of a desired indication. For example, the kit may also contain instructions for use of the spray pump or other delivery device.

In other embodiments, a pharmaceutical kit is provided and contains a medication which causes the formation of PF4 tetramers (e.g., heparin) in a first dosage unit and one or more of a PF4 antagonistic compound of the present application in a second dosage unit.

In yet other embodiments, a pharmaceutical kit is provided and contains a therapeutic agent which disrupts PF4 tetramers in a first dosage unit, one or more of a a PF4 antagonistic compound of the present disclosure in a second dosage unit, and one or more of the carriers or excipients described herein in a third dosage unit. The kit may optionally contain instructions for administering the components of the kit to a subject, for example, having cancer.

In yet a further embodiment, a pharmaceutical kit is provided and contains a therapeutic agent that causes formation of a PF4 tetramer in a first dosage unit (e.g., heparin), a therapeutic agent that disrupts a PF4 tetramer in a second dosage unit, and one or more of a PF4 antagonistic compound described herein in a third dosage unit, and one or more of the carriers or excipients in a fourth dosage unit. The kit may optionally contain instructions for administering the components of the kit to a subject, e.g., having cancer.

### Pharmaceutical Compositions and Formulations

The present application also provides pharmaceutical compositions comprising an effective amount of a compound of Formula (I) disclosed herein, or a pharmaceutically acceptable salt thereof; and a pharmaceutically acceptable carrier. In certain embodiments, the application also provides pharmaceutical compositions and dosage forms comprising any one the additional therapeutic agents described herein. The carrier(s) are “acceptable” in the sense of being compatible with the other ingredients of the formulation and, in the case of a pharmaceutically acceptable carrier, not deleterious to the recipient thereof in an amount used in the medicament.

Pharmaceutically acceptable carriers, adjuvants and vehicles that may be used in the pharmaceutical compositions of the present application include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

The compositions or dosage forms may contain any one of the compounds and therapeutic agents described herein in the range of 0.005% to 100% with the balance made up from the suitable pharmaceutically acceptable excipients. The contemplated compositions may contain 0.001%-100% of any one of the compounds and therapeutic agents provided herein, in some embodiments 0.1-95%, in other embodiments 75-85%, in further embodiments 20-80%, wherein the balance may be made up of any pharmaceutically acceptable excipient described herein, or any combination of these excipients.

### Routes of Administration and Dosage Forms

The pharmaceutical compositions of the present application include those suitable for any acceptable route of administration. Acceptable routes of administration include, but are not limited to, buccal, cutaneous, endocervical, endosinusial, endotracheal, enteral, epidural, interstitial, intra-abdominal, intra-arterial, intrabronchial, intrabursal, intracerebral, intracisternal, intracoronary, intradermal, intraductal, intraduodenal, intradural, intraepidermal, intraesophageal, intragastric, intragingival, intraileal, intralymphatic, intramedullary, intrameningeal, intramuscular, intranasal, intraovarian, intraperitoneal, intraprostatic, intrapulmonary, intrasinal, intraspinal, intrasynovial, intratesticular, intrathecal, intratubular, intratumoral, intrauterine, intravascular, intravenous, nasal, nasogastric, oral, parenteral, percutaneous, peridural, rectal, respiratory (inhalation), subcutaneous, sublingual, submucosal, topical, transdermal, transmucosal, transtracheal, ureteral, urethral and vaginal.

Compositions and formulations described herein may conveniently be presented in a unit dosage form, e.g., tablets, sustained release capsules, and in liposomes, and may be prepared by any methods well known in the art of pharmacy. See, for example, Remington: The Science and Practice of Pharmacy, Lippincott Williams & Wilkins, Baltimore, MD (20th ed. 2000). Such preparative methods include the step of bringing into association with the molecule to be administered ingredients such as the carrier that constitutes one or more accessory ingredients. In general, the compositions are prepared by uniformly and intimately bringing into association the active ingredients with liquid carriers, liposomes or finely divided solid carriers, or both, and then, if necessary, shaping the product.

In some embodiments, any one of the compounds and therapeutic agents disclosed herein are administered orally. Compositions of the present application suitable for oral administration may be presented as discrete units such as capsules, sachets, granules or tablets each containing a predetermined amount (e.g., effective amount) of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In the case of tablets for oral use, carriers that are commonly used include lactose, sucrose, glucose, mannitol, and silicic acid and starches. Other acceptable excipients may include: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. For oral administration in a capsule form, useful diluents include lactose and dried corn starch. When aqueous suspensions are administered orally, the active ingredient is combined with emulsifying and suspendfing agents. If desired, certain sweetening and/or flavoring and/or coloring agents may be added. Compositions suitable for oral administration include lozenges comprising the ingredients in a flavored basis, usually sucrose and acacia or tragacanth; and pastilles comprising the active ingredient in an inert basis such as gelatin and glycerin, or sucrose and acacia.

Compositions suitable for parenteral administration include aqueous and non-aqueous sterile injection solutions or infusion solutions which may contain antioxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, saline (e.g., 0.9% saline solution) or 5% dextrose solution, immediately prior to use. Extemporaneous injection solutions and suspensions may be prepared from sterile powders, granules and tablets. The injection solutions may be in the form, for example, of a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The pharmaceutical compositions of the present application may be administered in the form of suppositories for rectal administration. These compositions can be prepared by mixing a compound of the present application with a suitable non-irritating excipient which is solid at room temperature but liquid at the rectal temperature and therefore will melt in the rectum to release the active components. Such materials include, but are not limited to, cocoa butter, beeswax, and polyethylene glycols.

The pharmaceutical compositions of the present application may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, for example, U.S. Pat. No. 6,803,031. Additional formulations and methods for intranasal administration are found in Ilium, L., J Pharm Pharmacol, 56:3-17, 2004 and Ilium, L., Eur J Pharm Sci 11:1-18, 2000.

The topical compositions of the present disclosure can be prepared and used in the form of an aerosol spray, cream, emulsion, solid, liquid, dispersion, foam, oil, gel, hydrogel, lotion, mousse, ointment, powder, patch, pomade, solution, pump spray, stick, towelette, soap, or other forms commonly employed in the art of topical administration and/or cosmetic and skin care formulation. The topical compositions can be in an emulsion form. Topical administration of the pharmaceutical compositions of the present application is especially useful when the desired treatment involves areas or organs readily accessible by topical application. In some embodiments, the topical composition comprises a combination of any one of the compounds and therapeutic agents disclosed herein, and one or more additional ingredients, carriers, excipients, or diluents including, but not limited to, absorbents, anti-irritants, anti-acne agents, preservatives, antioxidants, coloring agents/pigments, emollients (moisturizers), emulsifiers, film-forming/holding agents, fragrances, leave-on exfoliants, prescription drugs, preservatives, scrub agents, silicones, skin-identical/repairing agents, slip agents, sunscreen actives, surfactants/detergent cleansing agents, penetration enhancers, and thickeners.

The compounds and therapeutic agents of the present application may be incorporated into compositions for coating an implantable medical device, such as prostheses, artificial valves, vascular grafts, stents, or catheters. Suitable coatings and the general preparation of coated implantable devices are known in the art and are exemplified in U.S. Pat. Nos. 6,099,562; 5,886,026; and 5,304,121. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccharides, polyethylene glycol, phospholipids or combinations thereof to impart controlled release characteristics in the composition. Coatings for invasive devices are to be included within the definition of pharmaceutically acceptable carrier, adjuvant or vehicle, as those terms are used herein.

According to other embodiments, the present application provides an implantable drug release device impregnated with or containing a compound or a therapeutic agent, or a composition comprising a compound of the present application or a therapeutic agent, such that said compound or therapeutic agent is released from said device and is therapeutically active.

### Dosages and Regimens

In the pharmaceutical compositions of the present application, a compound of Formula (I) is present in an effective amount (e.g., a therapeutically effective amount).

Effective doses may vary, depending on the diseases treated, the severity of the disease, the route of administration, the sex, age and general health condition of the subject, excipient usage, the possibility of co-usage with other therapeutic treatments such as use of other agents and the judgment of the treating physician.

In some embodiments, an effective amount of a compound of Formula (I) can range, for example, from about 0.001 mg/kg to about 500 mg/kg (e.g., from about 0.001 mg/kg to about 200 mg/kg; from about 0.01 mg/kg to about 200 mg/kg; from about 0.01 mg/kg to about 150 mg/kg; from about 0.01 mg/kg to about 100 mg/kg; from about 0.01 mg/kg to about 50 mg/kg; from about 0.01 mg/kg to about 10 mg/kg; from about 0.01 mg/kg to about 5 mg/kg; from about 0.01 mg/kg to about 1 mg/kg; from about 0.01 mg/kg to about 0.5 mg/kg; from about 0.01 mg/kg to about 0.1 mg/kg; from about 0.1 mg/kg to about 200 mg/kg; from about 0.1 mg/kg to about 150 mg/kg; from about 0.1 mg/kg to about 100 mg/kg; from about 0.1 mg/kg to about 50 mg/kg; from about 0.1 mg/kg to about 10 mg/kg; from about 0.1 mg/kg to about 5 mg/kg; from about 0.1 mg/kg to about 2 mg/kg; from about 0.1 mg/kg to about 1 mg/kg; or from about 0.1 mg/kg to about 0.5 mg/kg).

In some embodiments, an effective amount of a compound of Formula (I) is about 0.1 mg/kg, about 0.5 mg/kg, about 1 mg/kg, about 2 mg/kg, or about 5 mg/kg.

The foregoing dosages can be administered on a daily basis (e.g., as a single dose or as two or more divided doses, e.g., once daily, twice daily, thrice daily) or non-daily basis (e.g., every other day, every two days, every three days, once weekly, twice weekly, once every two weeks, once a month).

### EXAMPLES

### Materials and Methods

Preparative and analytical methods used to generate and evaluate the compounds of the following examples included the following:

LC/MS data (ESI+) were determined with a Waters Alliance 2695 HPLC/MS (Waters Symmetry C18, 4.6×75 mm, 3.5 μm) or (Phenomenex C18, 4.6×75 mm, 3.0 μm) with a 2996 diode array detector from 210-400 nm; the solvent system is 5-95% MeCN in water (with 0.1% TFA) over nine minutes using a linear gradient, and retention times are in minutes. Mass spectrometry was performed on a Waters ZQ using electrospray in positive mode.

LC/MS data (ESI−) were determined with a Shimadzu Prominence HPLC/MS (Phenomenex Luna C18, 3.0×50 mm, 3 μm) with a 2996 diode array detector from 210-400 nm; the solvent system is 5-95% MeCN in water (with 0.1% formic acid) over five minutes using a linear gradient, and retention times are in minutes. Mass spectrometry was performed on a Applied Biosystems MDS Sciex API 2000 using electrospray in negative mode. Alternatively LC/MS data (ESI−) were determined with a Waters Alliance 2695 HPLC/MS (Phenomenex C18, 4.6×75 mm, 3.0 μm) with a 2996 diode array detector from 210-400 nm; the solvent system is 5-95% MeCN in water (with 0.1% formic acid) over nine minutes using a linear gradient, and retention times are in minutes. Mass spectrometry was performed on a Waters ZQ using electrospray in positive mode.

HRMS data were determined by The University of Notre Dame Mass Spectrometry & Proteomics Facility on a Bruker micrOTOF II.

Preparative reversed phase HPLC was performed on a Waters Sunfire column (19×50 mm, C18, 5 μm) with a 10 min mobile phase gradient of 10% acetonitrile/water to 90% acetonitrile/water with 0.1% TFA as buffer using 214 and 254 nm as detection wavelengths. Injection and fraction collection were performed with a Gilson 215 liquid handling apparatus using Trilution LC software.

¹H NMR were recorded on Varian Oxford 300 MHz, in DMSO-d₆. Chemical shifts (δ) are expressed in ppm downfield from tetramethylsilane (TMS) unless otherwise noted.

Purities are generally ≥90% by NMR or LCMS except otherwise noted.

### Example 1—Synthesis of 5,5′-carbonylbis(2-acetyl-1h-indene-1,3(2h)-dione)

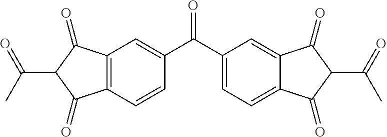 [SMILES: CC(=O)C1C(=O)c2ccc(C(=O)c3ccc4c(c3)C(=O)C(C(C)=O)C4=O)cc2C1=O]

The mixture of 4,4-carbonyldiphthalic anhydride (CAS Number: 2421-28-5; 100 mg, 0.31 mmol), pentane-2,4-dione (32 μL, 0.62 mmol) in Ac₂O (360 mg, 3.6 mmol) was treated with Et₃N (0.22 ml, 1.55 mmol). After the mixture was stirred for 1 hour at room temperature, 5 ml of 1 N HCl solution was added and stirred for 2 h. The resulting solid was filtered, washed with ether, hexanes and dried under vacuum. This crude product was reslurried in AcOEt and MeOH. The solid was collected and dried over 18 hours to give the title compound (75 mg, 60% yield). LC/MS (ESI+): R_{f}=5.36 min, (M+Na)⁺=425.01; LC/MS (ESI−): R_{f}=2.75 min, (M−H)⁻=401.7; HRMS (ESI−): calculated for C23H13O7 m/z [M−H]⁻: 401.066676, observed 401.066297; ¹H NMR: δ=8.10 (d, J=7.6 Hz, 2H) 7.96 (s, 2H), 7.88 (d, J=7.6 Hz, 2H), 2.52 (s, 6H).

### Example 2—Synthesis of 5,5′-sulfonylbis(2-acetyl-1h-indene-1,3(2h)-dione)

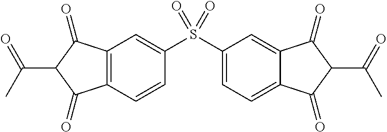 [SMILES: CC(=O)C1C(=O)c2ccc(S(=O)(=O)c3ccc4c(c3)C(=O)C(C(C)=O)C4=O)cc2C1=O]

The title compound was synthesized from the reaction of 5,5′-sulfonylbis(isobenzofuran-1,3-dione) (CAS number 2540-99-0; 358 mg, 1.0 mmol) and pentane-2,4-dione (206 μL, 2 mmol) with triethylamine and acetic anhydride according to the conditions described in Example 1 to give the title compound (112 mg, 26% yield). LC/MS (ESI+): R_{f}=5.14 min, HRMS (ESI−): calculated for C22H₁₃O8S m/z [M−H]⁻: 437.033662, observed 437.032434; ¹H NMR: δ=8.44-8.27 (m, 2H), 8.15 (d, J=0.9 Hz, 2H), 7.86 (d, J=7.6 Hz, 2H), 2.52 (s, 6H).

### Example 3—Synthesis of diethyl 5,5′-carbonylbis(1,3-dioxo-2,3-dihydro-1h-indene-2-carboxylate)

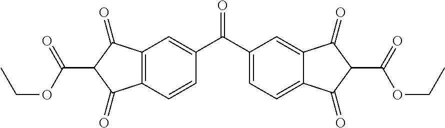 [SMILES: CCOC(=O)C1C(=O)c2ccc(C(=O)c3ccc4c(c3)C(=O)C(C(=O)OCC)C4=O)cc2C1=O]

The title compound was synthesized from the reaction of 4,4-carbonyldiphthalic anhydride (100 mg, 0.31 mmol) and ethyl 3-oxobutanoate (0.67 ml, 7.2 mmol) with trimethylamine and acetic anhydride according to the conditions provided in Example 1 to give the title compound (1.1 g, 79% yield). LC/MS (ESI+): R_{f}=3.31 min, no MI; ¹H NMR: δ=7.91-7.81 (m, 2H), 7.73-7.63 (m, 2H), 7.58 (d, J=7.3 Hz, 2H), 4.16-3.97 (m, 4H), 1.25-1.10 (m, 6H).

### Example 4—Synthesis of 5,5′-carbonylbis(1h-indene-1,3(2h)-dione)

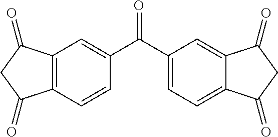 [SMILES: O=C(c1ccc2c(c1)C(=O)CC2=O)c1ccc2c(c1)C(=O)CC2=O]

A mixture of 4,4-carbonyldiphthalic anhydride (8.0 g, 24.83 mmol,), acetic anhydride (25 ml), and isopropyl 3-oxobutanoate (8.6 ml, 59.59 mmol, 2.4 equiv) was treated with Et₃N (13.8 ml, 99.31 mmol, 4.0 equiv). This stirring, the mixture quickly formed a homogeneous solution and was stirred for three days at room temperature. LCMS indicated formation of the bis isopropyl carboxylate intermediate. After, 150 ml of cold 2N HCl was added and the reaction mixture was heated under a gentle reflux for 12 hours. Decarboxylation was evident by reaction mixture forming bubbles. LCMS indicated complete conversion to the indanone species. This heterogeneous mixture was filtered and the resulting cake was washed with dilute HCl and water. Resulting tan solid was air dried overnight and added to 175 ml of MeOH and stirred for 24 hours. Contents were filtered and cake washed with MeOH and obtained solid was dried under vacuum to give the titled compound (4.4 g, 12.4 mmol, 90%) as a brown solid. LCMS (ESI−): R_{f}=4.10, (M−H)⁻=317.27, HRMS (ESI−): calculated for C19H₉₀₅ m/z [M−H]⁻: 317.045547, observed 317.044763; ¹H NMR: δ=8.40-8.22 (m, 4H), 8.16 (d, J=7.9 Hz, 2H), 3.37 (s, 4H)

### Example 5—Synthesis of 5-(2-acetyl-2-carbo-t-butoxy-2,3-dihydro-1,3dioxo-1h-inden-6-yloxy)-2-acetyl-2-carbo-t-butoxy-2,3-dihydro-1,3-dioxo-1h-indene

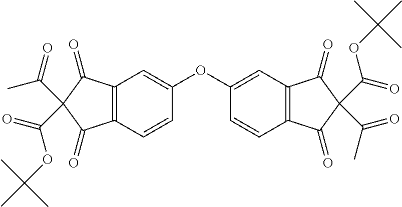 [SMILES: CC(=O)C1(C(=O)OC(C)(C)C)C(=O)c2ccc(Oc3ccc4c(c3)C(=O)C(C(C)=O)(C(=O)OC(C)(C)C)C4=O)cc2C1=O]

To 4,4′-Oxydi(phthalic anhydride) (CAS Number 1823-59-2, 310 mg, 1.0 mmol) and t-butyl 3-oxobutanoate (0.33 ml, 2.0 mmol) in Ac₂O (2.0 ml) was added Et₃N (0.56 ml, 4.0 mmol). After stirring for 18 hours, the reaction mixture was treated with ice (10 g) and conc. HCl (1.0 ml), and stirred for 25 min. The resulting solid was filtered and dried. This solid dissolved in CH₂Cl₂ (minimum) was purified by ISCO with 10-30%-70% AcOEt/Hexanes to give the above named product (52 mg, 8.8% yield). HRMS (ESI−): calculated for 32H₂₉₀₁₁ m/z [M−H]⁻: 589.171535, observed 589.170952. ¹H NMR (chloroform-d) δ=7.94-8.09 (m, 4H) 7.39 (dd, J=8.50, 2.05 Hz, 2H) 2.61 (s, 6H) −0.08-0.10 (m, 18H).

### Example 6—Synthesis of 2-acetyl-5-(1,3-dioxo-indan-5-yloxy)-1,3-dioxo-indan-2-carboxylic acid ethyl ester

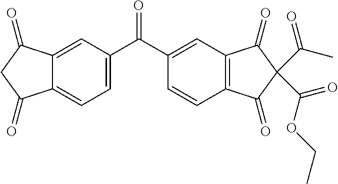 [SMILES: CCOC(=O)C1(C(C)=O)C(=O)c2ccc(C(=O)c3ccc4c(c3)C(=O)CC4=O)cc2C1=O]

To 4,4′-Oxydi(phthalic anhydride) (310 mg, 1.0 mmol) and ethyl 3-oxobutanoate (0.25 ml, 2.0 mmol) in Ac₂O (2.0 ml) was added Et₃N (0.56 ml, 4.0 mmol). After stirring for 18 hours, the reaction was treated with 1N HCl (10 ml), and stirred for 5 min. The resulting solid was filtered, dissolved in CH₂Cl₂ (2 ml) and purified by ISCO with 10-30% AcOEt/Hexanes to give a mixture which contained the desired products. This mixture was further purified by reverse phase HPLC (20-80% MeCN/H₂O) to give two products. Product 1 with retention time at 4.90 mins is the title compound (34 mg, 8% yield): HRMS (ESI−): calculated for C₂₃H₁₅O₈ m/z [M−H]⁻: 419.074855, observed 419.077241. ¹H NMR (chloroform-d) δ=7.99-8.14 (m, 3H) 7.51-7.62 (m, 1H) 7.35-7.50 (m, 2H) 4.26 (q, J=7.04 Hz, 2H) 3.28 (s., 2H) 2.62 (s, 3H) 1.28 (t, J=7.18 Hz, 3H).

### Example 7—Synthesis of 5-(2,3-dihydro-1,3dioxo-1h-inden-6-yloxy)-2,3-dihydro-1,3-dioxo-1h-indene

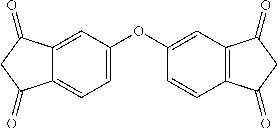 [SMILES: O=C1CC(=O)c2cc(Oc3ccc4c(c3)C(=O)CC4=O)ccc21]

To a mixture of 4,4′-oxydi(phthalic anhydride) (CAS Number 1823-59-2, 310 mg, 1.0 mmol) and methyl acetoacetate (0.22 ml, 2.1 mmol, 2.1 eq.) in acetic anhydride (2.0 ml) was added triethylamine (1.1 ml, 8 mmol, 8 eq.). The reaction was stirred at room temperature for 18 hours then water (18 ml) was added. The mixture was treated with concentrated hydrochloric acid (4 ml) and heated at 100° C. for 30 minutes. The reaction was cooled to room temperature and the solid was collected by filtration, washing with water. After drying under high vacuum, the solid was triturated in dichloromethane and hexanes were added. The solid was collected by filtration, washing with hexanes to obtain the product as an orangey-brown solid (228 mg, 74%). HRMS (ESI−): calculated for C₁₈H₉O₅ m/z [M−H]⁻: 305.045547, observed 305.046450; HRMS (ESI+): calculated for C₁₈H₁₁O₅ m/z [M−H]⁻: 307.060100, observed 307.060510. ¹H NMR (dichloromethane-d₂) δ=7.98-8.09 (m, 2H) 7.56 (dd, J=8.50, 2.35 Hz, 2H) 7.48 (d, J=2.35 Hz, 2H) 3.26 (s, 4H).

### Example 8—Synthesis of ethyl 2-acetyl-5-(1,3-dioxo-indan-5-yloxy)-1,3-dioxo-indan-2-carboxylic acid isopropyl ester

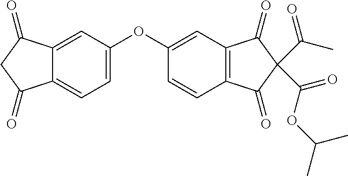 [SMILES: CC(=O)C1(C(=O)OC(C)C)C(=O)c2ccc(Oc3ccc4c(c3)C(=O)CC4=O)cc2C1=O]

To 4,4′-Oxydi(phthalic anhydride) (310 mg, 1.0 mmol) and isopropyl 3-oxobutanoate (0.3 ml, 2.0 mmol) in Ac₂O (2.0 ml) was added Et₃N (0.56 ml, 4.0 mmol). After stirring for 18 hrs, the reaction was treated with ice (10 g) and conc. HCl (1.0 ml), and stirred for 25 min. The resulting solid was filtered, dissolved in CH₂Cl₂ (minimum) and purified by ISCO with 10-30%-70% AcOEt/Hexanes to give a mixture which contained the desired products. This mixture was further purified by reversed phase HPLC (30-70% MeCN/H₂O) to give the title compound (5 mg). ¹H NMR (chloroform-d) δ=7.99-8.15 (m, 3H) 7.49-7.60 (m, 1H) 7.35-7.46 (m, 2H) 5.11 (dt, J=12.53, 6.19 Hz, 1H) 3.29 (s, 2H) 2.54-2.69 (m, 3H) 1.25 (d, J=6.45 Hz, 6H).

### Example 9—Synthesis of 5-(2-acetyl-2,3-dihydro-1,3-dioxo-1h-inden-6-yloxy)-2-acetyl-2,3-dihydro-1,3-dioxo-1h-indene

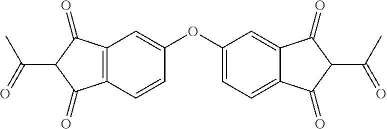 [SMILES: CC(=O)C1C(=O)c2ccc(Oc3ccc4c(c3)C(=O)C(C(C)=O)C4=O)cc2C1=O]

To 4,4′-Oxydi(phthalic anhydride) (310 mg, 1.0 mmol) and pentane-2,4-dione (0.2 ml, 2.0 mmol) in Ac₂O (2.0 ml) was added Et₃N (0.56 ml, 4.0 mmol). After stirring for 18 hrs, the reaction was treated with 1N HCl (10 ml), and stirred for 5 min. The resulting solid was filtered, dissolved in CH₂Cl₂ (2 ml) and purified by ISCO with 10-30% AcOEt/Hexanes to give a mixture which contained the desired products. This mixture was added DMF (2 ml), and stirred for 5 mins. The solid collected was washed by MeOH and dried to give the title product (retention time=5.66 mins, 45 mg). HRMS (ESI−): calculated for C₂₂H₁₃O₇ m/z [M−H]⁻: 389.066676, observed 389.064135. ¹H NMR δ=7.78-7.88 (m, 2H) 7.50 (dd, J=8.06, 2.20 Hz, 2H) 7.37 (d, J=2.05 Hz, 2H) 2.50 (s, 6H).

### Example 10—Synthesis of 2-acetyl-5-(2-acetyl-2,3-dihydro-1,3dioxo-1h-inden-5-yl)-2,3-dihydro-1,3-dioxo-1h-indene

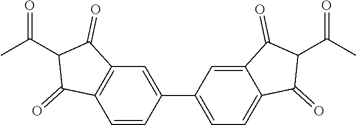 [SMILES: CC(=O)C1C(=O)c2ccc(-c3ccc4c(c3)C(=O)C(C(C)=O)C4=O)cc2C1=O]

To 4,4′-biphthalic anhydride (CAS Number 2420-87-3, 294 mg, 1.0 mmol) and pentane-2,4-dione (0.2 ml, 2.0 mmol) in Ac₂O (2.0 ml) was added Et₃N (0.56 ml, 4.0 mmol). After stirring for 18 hrs, the reaction was treated with 1N HCl (10 ml), and stirred for 5 min. The resulting solid was filtered, dried. This sold was then in MeOH (5 ml) and stirred for 20 mins. The desired product was collected and dried under vacuum for 18 hrs to give the title product (280 mg, 75% yield). HRMS (ESI−): calculated for C₂₂H₁₃O₆ m/z [M−H]⁻: 373.071762, observed 373.068892. ¹H NMR δ=8.56-8.62 (m, 1H) 8.47 (dd, J=7.92, 1.76 Hz, 1H) 8.15-8.25 (m, 2H) 8.08 (d, J=1.76 Hz, 1H) 7.86 (d, J=7.92 Hz, 1H) 2.54 (s, 6H).

### Example 11—Synthesis of 2-acetyl-5-[(2-acetyl-2,3-dihydro-1,3dioxo-1h-inden-5-yl)-1,1,1,3,3,3-hexafluoropropan-2-yl]-2,3-dihydro-1,3-dioxo-1h-indene

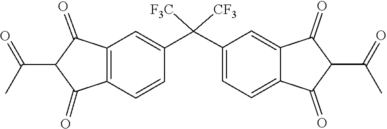 [SMILES: CC(=O)C1C(=O)c2ccc(C(c3ccc4c(c3)C(=O)C(C(C)=O)C4=O)(C(F)(F)F)C(F)(F)F)cc2C1=O]

To 4,4′-(hexafluoroisopro pylidene)diphthalic anhydride (CAS Number 1107-00-2, 444 mg, 1.0 mmol) and pentane-2,4-dione (0.25 ml, 2.0 mmol) in Ac₂O (2.0 ml) was added Et₃N (0.56 ml, 4.0 mmol). After stirring for 18 hrs, the reaction was treated with 1N HCl (10 ml), and stirred for 5 min. The resulting solid was filtered, dried and then dissolved in CH₂Cl₂ (3 ml) and purified by ISCO with 20% AcOEt/Hexanes to give the desired (220 mg, 42% yield). HRMS (ESI−): calculated for C₂₅H₁₃F₆O₆ m/z [M−H]⁻: 523.062181, observed 523.063833; ¹H NMR (dichloromethane-d₂) δ=7.87 (td, J=8.65, 3.52 Hz, 4H) 7.69-7.80 (m, 2H) 2.49-2.64 (m, 6H).

### Example 12—Synthesis of 5,5′-sulfonylbis(1h-indene-1,3(2h)-dione)

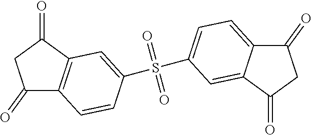 [SMILES: O=C1CC(=O)c2cc(S(=O)(=O)c3ccc4c(c3)C(=O)CC4=O)ccc21]

A mixture of 5,5′-sulfonylbis(isobenzofuran-1,3-dione) (5.0 g, 13.9 mmol,), acetic anhydride (15.5 ml), and isopropyl 3-oxobutanoate (4.8 ml, 33.5 mmol) was treated with Et₃N (7.6 ml, 55.8 mmol). This stirring mixture quickly formed a homogeneous solution and was stirred for three days. 150 ml of cold 2N HCl was then added and a solid came out of solution. The stirring rate was increased and flask was placed under a gentle reflux for 6 hours. LCMS indicated complete conversion to the indanone species. This heterogeneous mixture was filtered and the resulting cake was washed with dilute HCl and water. Resulting brown solid was air dried overnight and added to 100 ml of MeOH and stirred for 24 hours. Contents were filtered and cake washed with MeOH and obtained solid was dried under vacuum to give the titled compound (4.4 g, 12.4 mmol, 90%) as a brown solid. LCMS (ESI−): R_{f}=4.24, (M−H)⁻=352.90, HRMS (ESI−): calculated for C₁₈H₉O₆S m/z [M−H]⁻: 353.012533, observed 353.012716; ¹H NMR: δ=8.66-8.36 (m, 4H), 8.13 (d, J=8.2 Hz, 2H), 3.38 (s, 4H).

### Example 13—Synthesis of 5-(2,2,2-trifluoro-1-(5-indan-1,3-dionyl)-1-trifluoromethyl-ethyl)-indan-1,3-dione

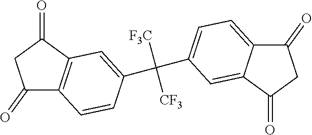 [SMILES: O=C1CC(=O)c2cc(C(c3ccc4c(c3)C(=O)CC4=O)(C(F)(F)F)C(F)(F)F)ccc21]

A mixture of 4,4′-(hexafluoroisopropylidene)diphthalic anhydride (2.0 g, 4.46 mmol,), acetic anhydride (6.2 ml), and isopropyl 3-oxobutanoate (1.5 ml, 10.7 mmol, 2.4 equiv) was treated with Et₃N (2.4 ml, 10.7 mmol, 2.4 equiv). This stirring mixture quickly formed a homogeneous solution and was stirred for three days. LCMS indicated formation of the bis isopropyl carboxylate intermediate. After, 75 ml of cold 2N HCl was added and a red tar precipitated from solution. This red tar was taken up in 100 ml of DCM and washed three times (50 ml) with cold 2N HCl. LCMS indicated complete conversion to the indanone species. The combine organic layers were dried over Na₂SO₄ and concentrated leaving behind the title compound as a red oil (1.76 g, 89%). LCMS (ESI−): R_{f}=5.03, (M−H)⁻=438.97, HRMS (ESI−): calculated for C₂₁H₁₀F₆O₄ m/z [M−H]⁻: 439.04105, observed 439.0563; ¹HNMR (chloroform-d): δ=7.98-8.1 (m, 4H), 7.81 (d, 2H), 3.30 (s, 2H).

### Example 14—Synthesis of 5,5′-carbonylbis(1,3-dioxo-indan-2-carboxylic acid phenylamide)

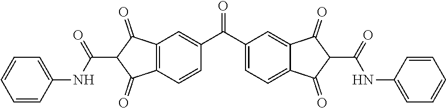 [SMILES: O=C(c1ccc2c(c1)C(=O)C(C(=O)Nc1ccccc1)C2=O)c1ccc2c(c1)C(=O)C(C(=O)Nc1ccccc1)C2=O]

To a solution of 5,5′-carbonylbis(1H-indene-1,3(2H)-dione) (prepared in Example 4, 64 mg, 0.2 mmol) in DMF (1.5 ml) at −78° C. was added Et₃N (85 μL, 0.46 mmol) and phenyl isocyanate (CAS Number 103-71-9, 48 μL, 0.44 mmol). The mixture was stirred for 2 h at this temperature, and warmed up to room temperature, then treated with 1N HCl solution (4 ml). The solid was filtered and washed by AcOEt and dried to give the title compound (91 mg, 82% yield). LC/MS (ESI+): R_{f}=6.04 min, (M+H)⁺=556.23; HRMS (ESI−): calculated for C₃₃H₁₉N₂O₇ m/z [M−H]⁻: 555.119775, observed 555.119873; ¹H NMR: δ=10.88-10.61 (m, 2H), 7.85 (d, J=6.7 Hz, 2H), 7.65 (s, 2H), 7.61-7.49 (m, 6H), 7.26 (t, J=7.9 Hz, 4H), 6.94 (t, J=7.3 Hz, 21H).

### Example 15—Synthesis of 5,5′-carbonylbis(1,3-dioxo-indan-2-carboxylic acid propylamide)

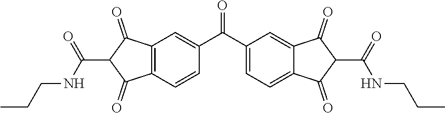 [SMILES: CCCNC(=O)C1C(=O)c2ccc(C(=O)c3ccc4c(c3)C(=O)C(C(=O)NCCC)C4=O)cc2C1=O]

In a manner similar to that described in Example 14, the title compound was synthesized from 5,5′-carbonylbis(1H-indene-1,3(2H)-dione) (64 mg, 0.2 mmol) and n-propyl isocyanate (CAS Number 110-78-1, 44 μL, 0.46 mmol) to give the title compound (32 mg, 33% yield). LC/MS (ESI+): R_{f}=6.42 min, (M+H)⁺=489.51; HRMS (ESI−): calculated for C₂₇H₂₃N₂O₇ m/z [M−H]⁻: 487.151075, observed 487.149660; ¹H NMR: δ=8.91 (br. s., 2H), 7.96 (d, J=7.6 Hz, 2H), 7.85-7.64 (m, 4H), 3.32 (br. s., 4H), 1.69-1.41 (m, 4H), 0.88 (t, J=7.5 Hz, 6H).

### Example 16—Synthesis of 5,5′-carbonylbis((1,3-dioxo-indane-2-carbonyl)-carbamic acid ethyl ester)

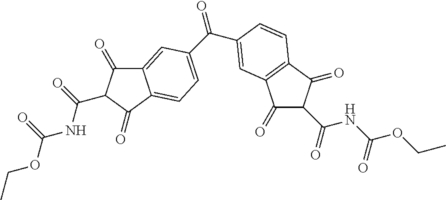 [SMILES: CCOC(=O)NC(=O)C1C(=O)c2ccc(C(=O)c3ccc4c(c3)C(=O)C(C(=O)NC(=O)OCC)C4=O)cc2C1=O]

In a manner similar to that described in Example 14, the title compound was prepared from 5,5′-carbonylbis(1H-indene-1,3(2H)-dione) (64 mg, 0.2 mmol) and ethoxycarbonyl isocyanate (CAS Number 19617-43-7, 170 μL, 0.92 mmol) to give the title compound (32 mg, 33% yield). LC/MS (ESI+): R_{f}=5.33 min, (M+2H)⁺=549.81; HRMS (ESI−): calculated for C₂₇H₂₀N₂O₁₁ m/z [M−H]⁻: 547.0944, observed 547.0944; ¹H NMR: δ=7.93 (d, J=7.6 Hz, 2H), 7.75 (s, 2H), 7.67 (d, J=7.6 Hz, 2H), 4.23-4.02 (m, 6H), 1.28-1.06 (m, 6H).

### Example 17—Synthesis of 5,5′-carbonylbis(1,3-dioxo-indan-2-carboxylic acid (2,6-dichloro-pyridin-4-yl)-amide)

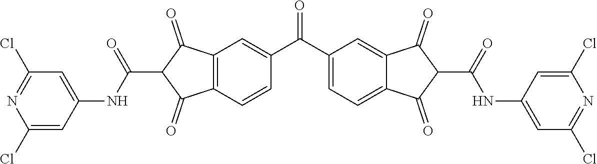 [SMILES: O=C(c1ccc2c(c1)C(=O)C(C(=O)Nc1cc(Cl)nc(Cl)c1)C2=O)c1ccc2c(c1)C(=O)C(C(=O)Nc1cc(Cl)nc(Cl)c1)C2=O]

To a mixture of 5,5′-carbonylbis(1H-indene-1,3(2H)-dione) (Example 4, 50 mg, 0.16 mmol) and 2,6-dichloro-4-isocyanatopyridine (CAS number 159178-03-7, 92 mg, 0.48 mmol) in DMF (2.0 ml) at −78° C. was added Et₃N (109 μL, 0.8 mmol). The mixture became solid. More DMF (1 ml) was added. The mixture was warmed up to RT for 18 hrs, then treated with 1N HCl (5 ml), stirred for 5 min. The solid was collected and washed by water and dried for 2 hrs. The solid was treated with water (4 ml), stirred, filtered and dried overnight to give the desired product (89 mg, 80% yield). HRMS (ESI−): calculated for C₃₁H₁₃C₁₄N₄O₇ m/z [M−H]⁻: 692.954383, observed 692.955101; ¹H NMR: δ=11.42 (s, 2H) 7.88 (d, J=7.62 Hz, 2H) 7.65-7.79 (m, 6H) 7.58 (d, J=7.33 Hz, 2H).

### Example 18—Synthesis of 5,5′-carbonylbis(1,3-dioxo-indan-2-carboxylic acid (2-benzo[1,3]dioxol-5-yl-ethyl)-amide)

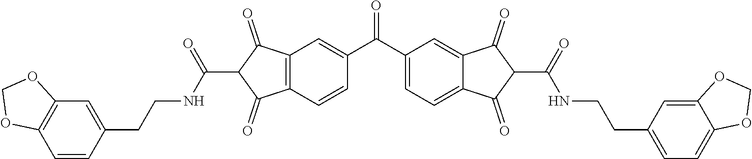 [SMILES: O=C(c1ccc2c(c1)C(=O)C(C(=O)NCCc1ccc3c(c1)OCO3)C2=O)c1ccc2c(c1)C(=O)C(C(=O)NCCc1ccc3c(c1)OCO3)C2=O]

To a stirred solution of 5,5′-carbonylbis(1H-indene-1,3(2H)-dione) (Example 4, 200 mg, 0.63 mmol) and DMF 5.0 ml in a dry ice acetone bath was added Et₃N (438.0 μl, 3.14 mmol, 5.0 equiv) over a 5 min period. After 5 min, 3,4-methylenedioxyphenethyl isocyanate (CAS Number 62334-09-2, 395 μl, 2.51 mmol, 4.0 equiv) was added dropwise over the course of 30 sec and the reaction vessel sealed. After 5 min the dry ice acetone bath was removed and contents was allowed to warm to room temperature and stirred for 18 hours. Once LCMS confirmed the reaction to be complete the reaction vessel was placed in an ice water bath. Then 2N HCl 10.0 ml was added and a solid precipitated. Stirring was maintained for an additional 30 min and then the solid was collected by filtration and washed with 25 ml of 2N HCl and 25 ml of Et₂O. The collected solid was air dried overnight then added to an excess of 1N NaOH (to basic pH) and 10 ml of CHCl₃ and sonicated for 10 min. The resulting suspension was filtered and was air dried overnight. The following day 200 mg of the dried material was placed in a soxhlet extractor and purged continuously with refluxing chloroform and dichloromethane for 24 hours. There was obtained 53.3 mg of a dark colored solid corresponding to a 29% yield of the bis sodium salt of the title compound. LCMS (ESI+): R_{f}=5.67, (M−H)⁺=701.64, HRMS (ESI−): calculated for C₃₉H₂₇N₂O₁₁ m/z [M−H]⁻: 699.162033, observed 699.161233; ¹H NMR: δ=8.38-8.59 (m, 2H), 7.71-7.86 (m, 2H), 7.50-7.60 (m, 2H), 7.43 (d, J=7.6 Hz, 2H), 6.72-6.87 (m, 4H), 6.65 (br d, J=8.2 Hz, 2H), 5.93 (s, 4H), 3.32-3.44 (m, 4H), 2.50-2.73 (m, 4H).

### Example 19—Synthesis of 5,5′-carbonylbis(1,3-dioxo-indan-2-carboxylic acid (furan-2-ylmethyl)-amide)

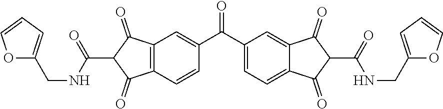 [SMILES: O=C(c1ccc2c(c1)C(=O)C(C(=O)NCc1ccco1)C2=O)c1ccc2c(c1)C(=O)C(C(=O)NCc1ccco1)C2=O]

The title compound was prepared as the bis-sodium salt in a similar manner to the synthesis of Example 18 from 5,5′-carbonylbis(1H-indene-1,3(2H)-dione) (300 mg, 0.93 mmol) and furfuryl isocyanate (CAS Number 71189-15-6, 400 μl, 3.72 mmol) to obtain a dark solid (57%). LCMS (ESI+): R_{f}=5.76, (M−H)⁺=565.49, HRMS (ESI−): calculated for C₃₁H₁₉N₂O₉ m/z [M−H]⁻: 563.109604, observed 563.110536; ¹H NMR: δ=8.76 (t, J=5.9 Hz, 2H), 7.67-7.89 (m, 2H), 7.55 (s, 4H), 7.45 (d, J=7.6 Hz, 2H), 6.29-6.38 (m, 2H), 6.22 (d, J=2.9 Hz, 2H), 4.34-4.48 (d, 4H).

### Example 20—Synthesis of 5,5′-carbonylbis(1,3-dioxo-indan-2-carboxylic acid (2-phenylethyl)-amide)

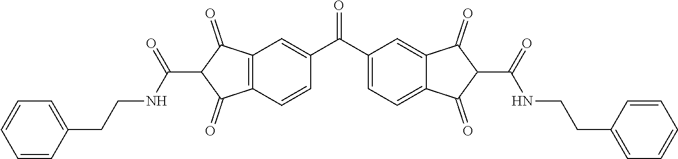 [SMILES: O=C(c1ccc2c(c1)C(=O)C(C(=O)NCCc1ccccc1)C2=O)c1ccc2c(c1)C(=O)C(C(=O)NCCc1ccccc1)C2=O]

The title compound was prepared as the bis-sodium salt in a similar manner to the synthesis of Example 18 from 5,5′-carbonylbis(1H-indene-1,3(2H)-dione) (200 mg, 0.63 mmol) and phenethyl isocyanate (CAS Number 1943-82-4, 304 μl, 2.20 mmol) to obtain a dark solid (39%). LCMS (ESI+): R_{f}=6.73, (M−H)⁺=613.63, HRMS (ESI−): calculated for C₃₇H₂₇N₂O₇ m/z [M−H]⁻: 611.182375, observed 611.184521; ¹H NMR: δ=8.48 (t, J=5.6 Hz, 2H), 7.72 (d, 2H), 7.44 (s, 2H), 7.35 (s, 2H), 7.03-7.29 (m, 10H), 3.31-3.44 (m, 4H), 2.63-2.75 (m, 4H).

### Example 21—Synthesis of 5,5′-carbonylbis(1,3-dioxo-indan-2-carboxylic acid (cyclopropyl)-amide)

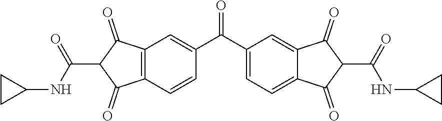 [SMILES: O=C(c1ccc2c(c1)C(=O)C(C(=O)NC1CC1)C2=O)c1ccc2c(c1)C(=O)C(C(=O)NC1CC1)C2=O]

The title compound was prepared as the bis-sodium salt in a similar manner to the synthesis of Example 18 from 5,5′-carbonylbis(1H-indene-1,3(2H)-dione) (300 mg, 0.94 mmol) and cyclopropyl isocyanate (CAS Number 56601-42-4, 197 μl, 2.83 mmol, 3.0 equiv) to obtain a dark solid (38%). LCMS (ESI+): R_{f}=5.63, (M−H)⁺=485.23, HRMS (ESI−): calculated for C₂₇H₁₉N₂O₇ m/z [M−H]⁻: 483.119775, observed 483.118839; ¹H NMR: δ=8.46 (d, J=4.1 Hz, 2H), 7.75 (d, J=7.5 Hz, 2H), 7.49 (s, 2H), 7.39 (d, J=7.6 Hz, 2H), 2.64 (td, J=7.0, 3.5, 2H), 0.57-0.64 (m, 4H), 0.32-0.38 (m, 4H).

### Example 22—Synthesis of 5,5′-carbonylbis(1,3-dioxo-indan-2-carboxylic acid (2-(4-fluorophenyl)ethyl)-amide)

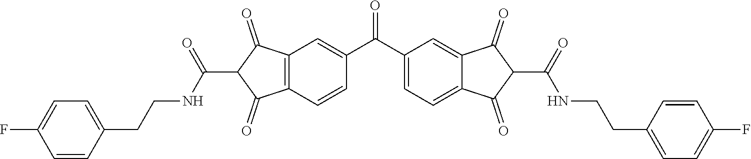 [SMILES: O=C(c1ccc2c(c1)C(=O)C(C(=O)NCCc1ccc(F)cc1)C2=O)c1ccc2c(c1)C(=O)C(C(=O)NCCc1ccc(F)cc1)C2=O]

The title compound was prepared as the bis-sodium salt in a similar manner to the synthesis of Example 18 from 5,5′-carbonylbis(1H-indene-1,3(2H)-dione) (300 mg, 0.94 mmol) and 4-fluorophenethyl isocyanate (CAS Number 1195-45-5, 412 μl, 2.83 mmol) to obtain a dark solid (33%). LCMS (ESI+): R_{f}=6.58, (M−H)⁺=649.60, HRMS (ESI−): calculated for C₃₇H₂₅F₂N₂O₇ m/z [M−H]⁻: 647.163531, observed 647.163586; ¹H NMR: δ=8.56-8.67 (m, 2H), 7.81-7.91 (m, 2H), 7.59-7.65 (m, 2H), 7.48-7.57 (m, 2H), 7.29-7.40 (m, 4H), 7.10-7.25 (m, 4H), 3.45-3.58 (m, 4H), 2.78-2.89 (m, 4H).

### Example 23—Synthesis of 5,5′-carbonylbis(1,3-dioxo-indan-2-carboxylic acid (3-methoxyphenyl)-amide)

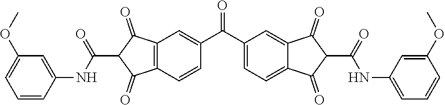 [SMILES: COc1cccc(NC(=O)C2C(=O)c3ccc(C(=O)c4ccc5c(c4)C(=O)C(C(=O)Nc4cccc(OC)c4)C5=O)cc3C2=O)c1]

The title compound was prepared as the bis-sodium salt in a similar manner to the synthesis of Example 18 from 5,5′-carbonylbis(1H-indene-1,3(2H)-dione) (200 mg, 0.63 mmol) and 3-methoxyphenyl isocyanate (CAS Number 18908-07-1, 325 μl, 2.52 mmol) to obtain a dark solid (45%). LCMS (ESI+): R_{f}=5.81, (M−H)⁺=617.57, HRMS (ESI−): calculated for C₃₅H₂₃N₂O₉ m/z [M−H]⁻: 615.140904, observed 615.140437; ¹H NMR; 5=10.71 (s, 2H), 7.81-7.96 (m, 2H), 7.61-7.71 (m, 2H), 7.48-7.59 (m, 2H), 7.35-7.46 (m, 2H), 7.00-7.19 (m, 4H), 6.43-6.59 (m, 2H), 3.71 (s, 6H).

### Example 24—Synthesis of 5,5′-carbonylbis(1,3-dioxo-indan-2-carboxylic acid (3-(phenyl)propyl)-amide)

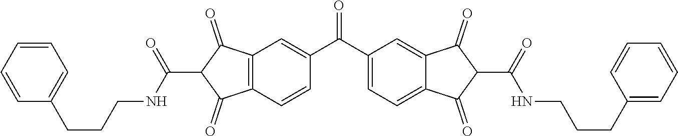 [SMILES: O=C(c1ccc2c(c1)C(=O)C(C(=O)NCCCc1ccccc1)C2=O)c1ccc2c(c1)C(=O)C(C(=O)NCCCc1ccccc1)C2=O]

The title compound was prepared as the bis-sodium salt in a similar manner to the synthesis of Example 18 from 5,5′-carbonylbis(1H-indene-1,3(2H)-dione) (200 mg, 0.63 mmol) and 3-phenylpropyl isocyanate (CAS Number 68664-23-3, 291 μl, 1.89 mmol) to obtain a dark solid (31%). LCMS (ESI+): R_{f}=7.05, (M−H)⁺=641.68, HRMS (ESI−): calculated for C₃₉H₃₁N₂O₇ m/z [M−H]⁻: 639.213675, observed 639.213532; ¹H NMR (DMSO-d6) δ: 8.47-8.62 (m, 2H), 7.67-7.86 (m, 2H), 7.48-7.57 (m, 2H), 7.35-7.48 (m, 2H), 6.91-7.31 (m, 10H), 3.16 (br s, 4H), 2.49-2.65 (m, 4H), 1.60-1.81 (m, 4H).

### Example 25—Synthesis of 5,5′-carbonylbis(1,3-dioxo-indan-2-carboxylic acid ((phenyl)methyl)-amide)

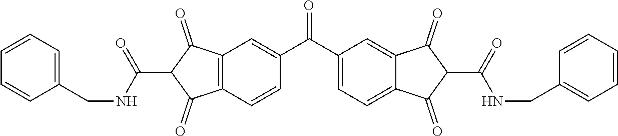 [SMILES: O=C(c1ccc2c(c1)C(=O)C(C(=O)NCc1ccccc1)C2=O)c1ccc2c(c1)C(=O)C(C(=O)NCc1ccccc1)C2=O]

The title compound was prepared as the bis-sodium salt in a similar manner to the synthesis of Example 18 from 5,5′-carbonylbis(1H-indene-1,3(2H)-dione) (300 mg, 0.93 mmol) and benzyl isocyanate (CAS Number 622-78-6, 349 μl, 2.83 mmol) to obtain a dark solid (51%). LCMS (ESI+): R_{f}=6.39, (M−H)⁺=585.13, HRMS (ESI−): calculated for C₃₅H₂₃N₂O₇ m/z [M−H]⁻: 583.151075, observed 583.149671; ¹H NMR: δ 8.43 (t, J=5.9 Hz, 2H), 7.34 (d, J=7.6 Hz, 2H), 7.11 (s, 2H), 7.01 (d, J=7.6 Hz, 2H), 6.69-6.90 (m, 10H), 3.98 (d, J=6.4 Hz, 4H).

### Example 26—Synthesis of 5,5′-carbonylbis(1,3-dioxo-indan-2-carboxylic acid ((4-fluorophenyl)methyl)-amide)

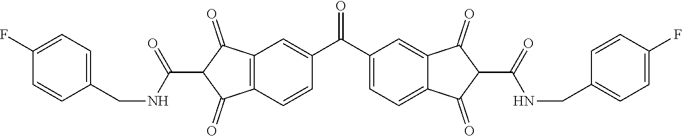 [SMILES: O=C(c1ccc2c(c1)C(=O)C(C(=O)NCc1ccc(F)cc1)C2=O)c1ccc2c(c1)C(=O)C(C(=O)NCc1ccc(F)cc1)C2=O]

The title compound was prepared as the bis-sodium salt in a similar manner to the synthesis of Example 18 from 5,5′-carbonylbis(1H-indene-1,3(2H)-dione) (300 mg, 0.93 mmol) and 4-fluorobenzyl isocyanate (CAS Number 3173-56-6, 361 μl, 2.83 mmol) to obtain a dark solid (22%). LCMS (ESI+): R_{f}=6.49, (M−H)⁺=621.55, HRMS (ESI−): calculated for C₃₅H₂₁F₂N₂O₇ m/z [M−H]⁻: 619.132231, observed 619.132231; ¹H NMR: δ=8.89 (t, J=5.9 Hz, 2H), 7.81 (d, J=7.5 Hz, 2H), 7.57 (d, J=1.2 Hz, 2H), 7.47 (d, J=7.0 Hz, 2H), 7.32 (dd, J=8.8, 5.9 Hz, 4H), 7.02-7.17 (m, 4H), 4.42 (br d, J=6.4 Hz, 4H).

### Example 27—Synthesis of 5,5′-carbonylbis(3-[(1,3-dioxo-indane-2-carbonyl)-amino]-propionic acid ethyl ester)

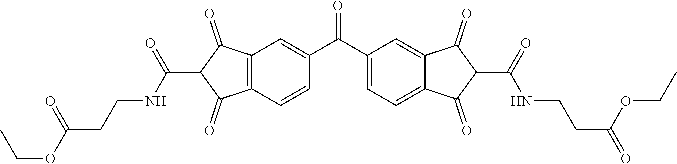 [SMILES: CCOC(=O)CCNC(=O)C1C(=O)c2ccc(C(=O)c3ccc4c(c3)C(=O)C(C(=O)NCCC(=O)OCC)C4=O)cc2C1=O]

The title compound was prepared as the bis-sodium salt in a similar manner to the synthesis of Example 18 from 5,5′-carbonylbis(1H-indene-1,3(2H)-dione) (300 mg, 0.93 mmol) and ethyl 3-isocyanatopropionate (CAS Number 5100-34-5, 490 μl, 3.72 mmol) to obtain a dark solid (45%). LCMS (ESI+): R_{f}=5.61, (M−H)⁺=605.55, HRMS (ESI−): calculated for C₃₁H₂₇N₂O₁₁ m/z [M−H]⁻: 603.162033, observed 603.161309; ¹H NMR: δ=: 8.49-8.75 (bt, 2H), 7.83 (d, 2H), 7.58 (s, 2H), 7.49 (d, 2H), 4.10 (q, 4H), 3.46 (bq, 4H), 2.52 (m, 4H), 1.21 (ts, 6H).

### Example 28—Synthesis of 5,5′-carbonylbis(1,3-dioxo-indan-2-carboxylic acid (2-methoxyphenyl)-amide)

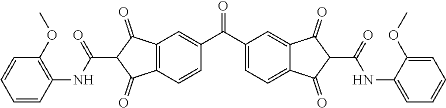 [SMILES: COc1ccccc1NC(=O)C1C(=O)c2ccc(C(=O)c3ccc4c(c3)C(=O)C(C(=O)Nc3ccccc3OC)C4=O)cc2C1=O]

The title compound was prepared as the bis-sodium salt in a similar manner to the synthesis of Example 18 from 5,5′-carbonylbis(1H-indene-1,3(2H)-dione) (200 mg, 0.63 mmol) and 2-methoxyphenyl isocyanate (325 μl, 2.52 mmol) to obtain a dark solid (25%). LCMS (ESI+): R_{f}=7.09, (M−H)⁺=617.57, HRMS (ESI−): calculated for C₃₅H₂₃N₂O₉ m/z [M−H]⁻: 615.140904, observed 615.140140; ¹H NMR: δ=10.83 (s, 2H), 8.40 (d, 2H), 7.75 (d, J=1.6 Hz, 2H), 7.53 (d, J=1.5 Hz, 2H), 7.43 (d, J=7.5 Hz, 2H), 6.64-6.93 (m, 6H), 3.76 (s, 6H).

### Example 29—Synthesis of 5,5′-carbonylbis(1,3-dioxo-indan-2-carboxylic acid (n-(2,3-dihydro-benzo[1,4]dioxin-6-yl))-amide)

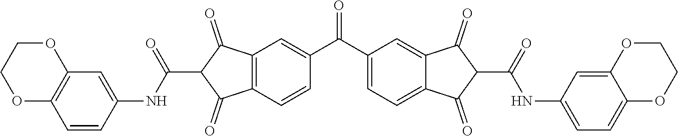 [SMILES: O=C(c1ccc2c(c1)C(=O)C(C(=O)Nc1ccc3c(c1)OCCO3)C2=O)c1ccc2c(c1)C(=O)C(C(=O)Nc1ccc3c(c1)OCCO3)C2=O]

The title compound was prepared as the bis-sodium salt in a similar manner to the synthesis of Example 18 from 5,5′-carbonylbis(1H-indene-1,3(2H)-dione) (100 mg, 0.31 mmol) and 3,4-(ethylenedioxy)phenyl isocyanate (128 μl, 0.94 mmol) to obtain a dark solid (70%). LCMS (ESI+): R_{f}=7.09, (M−H)⁺=673.59, HRMS (ESI−): calculated for C₃₇H₂₃N₂O₁₁ m/z [M−H]⁻: 671.130733, observed 671.130639; ¹H NMR: δ=10.60 (s, 2H), 7.86 (d, 2H), 7.62 (s, 2H), 7.52 (d, J=7.6 Hz, 2H), 7.33-7.44 (m, 2H), 6.85 (dd, J=8.8, 2.3 Hz, 2H), 6.73 (d, J=8.8 Hz, 2H), 4.05-4.27 (m, 8H).

### Example 30—Synthesis of 5,5′-5,5′-carbonylbis([(1,3-dioxo-indane-2-carbonyl)-amino]-acetic acid ethyl ester)

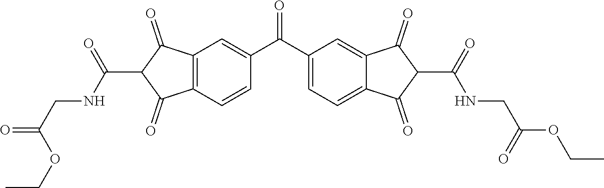 [SMILES: CCOC(=O)CNC(=O)C1C(=O)c2ccc(C(=O)c3ccc4c(c3)C(=O)C(C(=O)NCC(=O)OCC)C4=O)cc2C1=O]

The title compound was prepared as the bis-sodium salt in a similar manner to the synthesis of Example 18 from 5,5′-carbonylbis(1H-indene-1,3(2H)-dione) (300 mg, 0.93 mmol) and isocyanato-acetic acid ethyl ester (CAS Number 2949-22-6, 417 μl, 3.72 mmol) to obtain a dark solid (43%). LCMS (ESI+): R_{f}=5.15, (M−H)⁺=577.50, HRMS (ESI−): calculated for C₂₉H₂₃N₂O₁₁ m/z [M−H]⁻: 575.130733, observed 575.134244; ¹H NMR: δ=8.77 (t, J=5.9 Hz, 2H), 7.83 (t, 2H), 7.59 (d, J=1.2 Hz, 2H), 7.49 (d, J=8.2 Hz, 2H), 4.10 (q, 4H), 4.00 (d, 4H), 1.19 (t, J=7.0 Hz, 6H).

### Example 31—Synthesis of 5,5′-carbonylbis(1,3-dioxo-indan-2-carboxylic acid (4-methoxyphenyl)-amide)

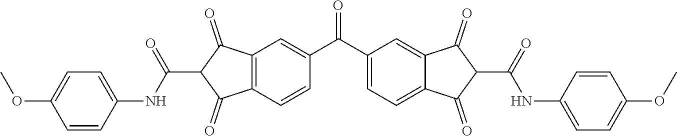

The title compound was prepared as the bis-sodium salt in a similar manner to the synthesis of Example 18 from 5,5′-carbonylbis(1H-indene-1,3(2H)-dione) (300 mg, 0.94 mmol) and 4-methoxyphenyl isocyanate (CAS Number 5416-93-3, 488 μl, 3.77 mmol) to obtain a dark solid (24%). LCMS (ESI+): R_{f}=6.47, (M−H)⁺=617.57,; HRMS (ESI−): calculated for C₃₅H₂₃N₂O₉ m/z [M−H]⁻: 615.140904, observed 615.140122; ¹H NMR: δ=10.52 (s, 2H), 7.80 (d, 2H), 7.58 (s, 2H), 7.43-7.52 (m, 6H), 6.75 (br d, J=8.8 Hz, 4H), 3.63 (s, 6H).

### Example 32—Synthesis of 5,5′-carbonylbis(3-[(1,3-dioxo-indane-2-carbonyl)-amino]-propionic acid)

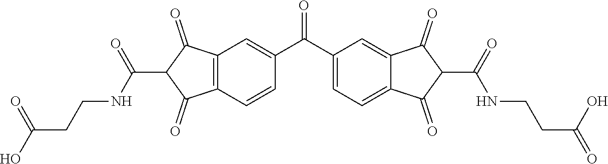 [SMILES: O=C(O)CCNC(=O)C1C(=O)c2ccc(C(=O)c3ccc4c(c3)C(=O)C(C(=O)NCCC(=O)O)C4=O)cc2C1=O]

To a 25 ml pear shaped flask there was 5,5′-carbonylbis(3-[(1,3-dioxo-indane-2-carbonyl)-amino]-propionic acid ethyl ester) (Example 27, 250 mg, 413 mmol) and a large excess of 1N NaOH. Almost right away a black solution formed with a mild exotherm. Stirring was maintained for 30 min and resulting solution was filtered. The filtrate was lyophilized down to a black solid. Next 15 ml of 2N HCl was added. The newly formed solid was filtered and air dried overnight to obtain the titled compound. LCMS (ESI+): R_{f}=3.95, (M−H)⁺=549.45, %; HRMS (ESI−): calculated for C₂₇H₁₉N₂O₁₁ m/z [M−H]⁻: 547.099433, observed 547.098408; ¹H NMR: δ=8.94 (br s, 2H), 8.03 (bd, 2H), 7.87 (bs, 2H), 7.78 (bd, 2H), 3.56-3.70 (m, 4H), 2.59-2.70 (bt, 4H).

### Example 33—Synthesis of 5,5′-sulphonylbis(1,3-dioxo-indan-2-carboxylic acid (2-phenylethyl)-amide)

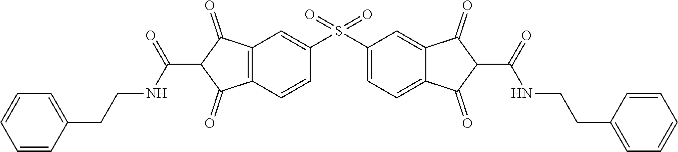 [SMILES: O=C(NCCc1ccccc1)C1C(=O)c2ccc(S(=O)(=O)c3ccc4c(c3)C(=O)C(C(=O)NCCc3ccccc3)C4=O)cc2C1=O]

A suspension of 5,5′-sulfonylbis(1H-indene-1,3(2H)-dione) (Example 12, 300 mg, 0.85 mmol) in DMF 7.0 ml was sonicated for 5 min for complete solution formation. The solution was stirred for 5 minutes in a dry ice acetone bath and then Et₃N (413.0 μl, 2.96 mmol, 3.5 equiv) was added. After and additional 5 minutes phenethyl isocyanate (351 μl, 2.54 mmol, 3.0 equiv) was added dropwise over the course of 30 sec. After 5 min the dry ice acetone bath was removed and contents stirred for 18 hours. Once LCMS confirmed the reaction to be complete the reaction vessel was placed in a ice water bath. Then 2N HCl 15.0 ml was added and a solid fell from solution. Stirring was maintained for a 30 min and then filtered. The solid was washed with 25 ml of 2N HCl and 25 ml of Et₂O and then air dried overnight. The solid was then added to an excess of 1 N NaOH (basic solution according to pH) and 10 ml of CHCl₃ and sonicated for 10 min. The resulting suspension was filtered and air dried overnight. The following day 200 mg of the dried material was placed on a Soxhlet extractor using 1:1 DCM/CHCl₃ to wash for 24 hours. There was obtained 166 mg of a dark colored solid corresponding to a 45% yield of the bis sodium salt of the title compound. LCMS (ESI+): R_{f}=6.47, (M−H)⁺=649.17, HRMS (ESI−): calculated for C₃₆H₂₇N₂O₈S m/z [M−H]⁻: 647.149360, observed 647.151684; ¹H NMR: δ=8.47 (br t, J=5.6 Hz, 2H), 8.07 (d, 2H), 7.66 (s, 2H), 7.48 (d, 2H), 7.10-7.37 (m, 10H), 3.40-3.46 (m, 4H), 2.64-2.83 (t, 4H).

### Example 34—Synthesis of 5,5′-sulphonylbis(1,3-dioxo-indan-2-carboxylic acid (cyclopropyl)-amide)

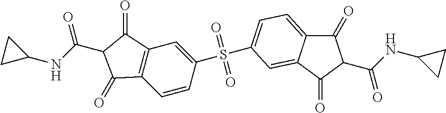 [SMILES: O=C(NC1CC1)C1C(=O)c2ccc(S(=O)(=O)c3ccc4c(c3)C(=O)C(C(=O)NC3CC3)C4=O)cc2C1=O]

The title compound was prepared as the bis-sodium salt in a similar manner to the synthesis of Example 33 from 5,5′-sulfonylbis(1H-indene-1,3(2H)-dione) (300 mg, 0.85 mmol) and cyclopropyl isocyanate (177 μl, 2.54 mmol) to obtain a dark colored solid (41%). LCMS (ESI+): R_{f}=5.28, (M−H)⁺=521.01, HRMS (ESI−): calculated for C₂₆H₁₉N₂O₈S m/z [M−H]⁻: 519.086760, observed 519.085886; ¹H NMR: δ=8.28 (d, J=4.1 Hz, 2H), 7.87-7.96 (m, 2H), 7.51 (d, J=1.2 Hz, 2H), 7.35 (d, J=7.6 Hz, 2H), 2.48-2.61 (m, 2H), 0.42-0.57 (m, 4H), 0.20-0.31 (m, 4H).

### Example 35—Synthesis of 5,5′-sulphonylbis(1,3-dioxo-indan-2-carboxylic acid (4-methoxyphenyl)-amide)

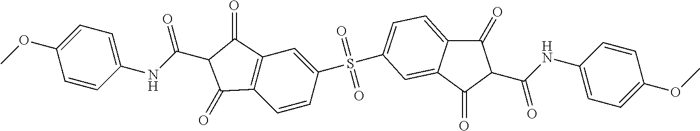

The title compound was prepared as the bis-sodium salt in a similar manner to the synthesis of Example 33 from 5,5′-sulfonylbis(1H-indene-1,3(2H)-dione) (300 mg, 0.85 mmol) and 4-methoxyphenyl isocyanate (438 μl, 3.39 mmol) to obtain a dark colored solid (28%). LCMS (ESI+): R_{f}=4.37, (M−H)⁺=653.71, HRMS (ESI−): calculated for C₃₄H₂₃N₂O₁₀S m/z [M−H]⁻: 651.107890, observed 651.106801; ¹H NMR: δ=10.45 (s, 2H), 8.05 (d, 2H), 7.66 (s, 2H), 7.48 (s, 2H) 7.42 (d, 4H), 6.77 (d, J=9.4 Hz, 4H), 3.64 (s, 6H).

### Example 36—Synthesis of 5,5′-sulphonylbis(1,3-dioxo-indan-2-carboxylic acid (3-methoxyphenyl)-amide)

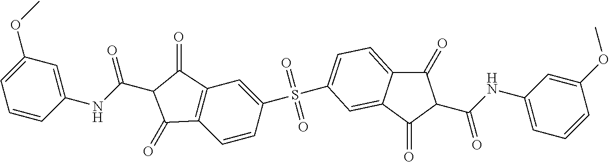 [SMILES: COc1cccc(NC(=O)C2C(=O)c3ccc(S(=O)(=O)c4ccc5c(c4)C(=O)C(C(=O)Nc4cccc(OC)c4)C5=O)cc3C2=O)c1]

The title compound was prepared as the bis-sodium salt in a similar manner to the synthesis of Example 33 from 5,5′-sulfonylbis(1H-indene-1,3(2H)-dione) (300 mg, 0.85 mmol) and 3-methoxyphenyl isocyanate (146 μl, 1.13 mmol) to obtain a dark colored solid (37%). LCMS (ESI+): R_{f}=4.37, (M−H)⁺=653.71, HRMS (ESI−): calculated for C₃₄H₂₃N₂O₁₀S m/z [M−H]⁻: 651.107890, observed 651.108678; ¹H NMR: δ=10.63 (s, 2H), 8.06 (d, 2H), 7.68 (d, J=1.8 Hz, 2H), 7.51 (d, J=7.6 Hz, 2H), 7.35 (t, J=2.1 Hz, 2H), 7.03-7.13 (m, 2H), 6.95 (d, 2H), 6.45 (d, 2H), 3.66 (s, 6H).

### Example 37—Synthesis of 5,5′-sulphonylbis(1,3-dioxo-indan-2-carboxylic acid (phenyl)-amide)

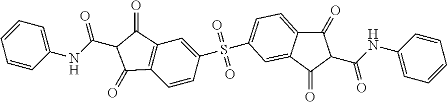 [SMILES: O=C(Nc1ccccc1)C1C(=O)c2ccc(S(=O)(=O)c3ccc4c(c3)C(=O)C(C(=O)Nc3ccccc3)C4=O)cc2C1=O]

The title compound was prepared as the bis-sodium salt in a similar manner to the synthesis of Example 33 from 5,5′-sulfonylbis(1H-indene-1,3(2H)-dione) (300 mg, 0.85 mmol) and phenyl isocyanate (368 μl, 3.39 mmol) to obtain a dark colored solid (31%). LCMS (ESI+): R_{f}=3.92, (M−H)⁺=593.57, HRMS (ESI−): calculated for C₃₂H₁₉N₂O₈S m/z [M−H]⁻: 591.086760, observed 591.084822; ¹H NMR: δ=10.67 (s, 2H), 8.05-8.16 (m, 2H), 7.74 (s, 2H), 7.50-7.63 (m, 6H), 7.24 (t, J=7.6 Hz, 4H), 6.84-7.00 (m, 2H).

### Example 38—Synthesis of 5,5′-carbonylbis(1,3-dioxo-n-(m-tolyl)-2,3-dihydro-1h-indene-2-carboxamide)

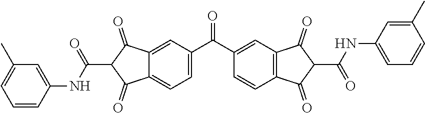 [SMILES: Cc1cccc(NC(=O)C2C(=O)c3ccc(C(=O)c4ccc5c(c4)C(=O)C(C(=O)Nc4cccc(C)c4)C5=O)cc3C2=O)c1]

The title compound was prepared in a similar manner to the synthesis of Example 18 from 5,5′-carbonylbis(1H-indene-1,3(2H)-dione) (300 mg, 0.94 mmol) and 4-methphenyl isocyanate (CAS Number 622-58-2, 512.2 μl, 3.72 mmol) to obtain a dark solid bis sodium salt (256, 44%). LCMS R_{f}=5.12; HRMS (ESI−): calculated for C₃₅H₂₃N₂O₇ m/z [M−H]⁻: 583.1511, observed 583.1496; ¹H NMR δ: 10.72 (s, 2H), 7.83-7.91 (m, 2H), 7.65 (s, 2H), 7.51-7.59 (m, 2H), 7.39-7.50 (m, 4H), 7.12 (t, J=7.6 Hz, 2H), 6.70-6.82 (m, 2H), 2.29 (s, 6H).

### Example 65—Synthesis of 5,5′-carbonylbis(n-allyl-1,3-dioxo-2,3-dihydro-1h-indene-2-carboxamide)

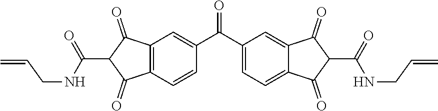 [SMILES: C=CCNC(=O)C1C(=O)c2ccc(C(=O)c3ccc4c(c3)C(=O)C(C(=O)NCC=C)C4=O)cc2C1=O]

The title compound was prepared in a similar manner to the synthesis of Example 18 from 5,5′-carbonylbis(1H-indene-1,3(2H)-dione) (150 mg, 0.47 mmol) and allyl isocyanate (CAS Number 1476-23-9, 116 μL, 1.2 mmol) to obtain a dark solid bis sodium salt (39 mg, 17%). LC/MS (ESI+): R_{f}=5.50 min, (M+H)⁺=485.04; HRMS (ESI−): calculated for C₂₇H₂₀N₂O₇ m/z [M−H]⁻: 483.1198, observed 483.1198; ¹H NMR δ 8.58 (t, J=5.27 Hz, 2H), 7.79 (d, J=7.62 Hz, 2H), 7.54 (s, 2H), 7.44 (d, J=7.62 Hz, 2H), 5.81-5.95 (m, 2H), 5.15 (d, J=16.99 Hz, 2H), 5.04 (d, J=9.96 Hz, 2H), 3.84 (br. s., 4H).

### Example 66—Synthesis of 5,5′-carbonylbis(n-(2-(2-methoxyethoxy)ethyl)-1,3-dioxo-2,3-dihydro-1h-indene-2-carboxamide)

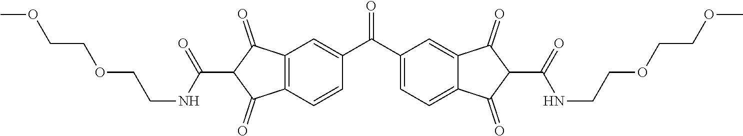

The title compound was prepared to the synthesis of Example 18, except the sodium salt formation was omitted, from 5,5′-carbonylbis(1H-indene-1,3(2H)-dione) (150 mg, 0.47 mmol) and 1-isocyanato-2-(2-methoxyethoxy)ethane (CAS Number 90426-82-7, 122 μL, 1.2 mmol) to obtain a dark solid (177 mg, 62%). LC/MS (ESI+): R_{f}=4.57 min, (M+H)⁺=609.17; HRMS (ESI−): calculated for C₃₁H₃₂N₂O₁₁ m/z [M−H]⁻: 607.1933, observed 607.1933; ¹H NMR δ 8.61-9.08 (m, 2H), 7.99 (t, J=6.60 Hz, 2H), 7.82 (s, 2H), 7.70 (d, J=5.40 Hz, 2H), 3.48-3.64 (m, 12H), 3.37-3.46 (m, 4H), 3.22 (s, 6H).

### Example 67—Synthesis of 5,5′-carbonylbis(1,3-dioxo-n-(2-(2,2,2-trifluoroethoxy) ethyl)-2,3-dihydro-1h-indene-2-carboxamide)

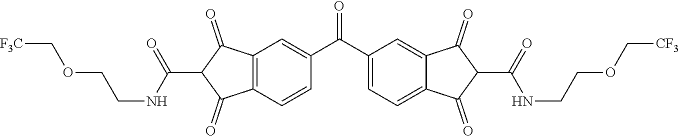 [SMILES: O=C(c1ccc2c(c1)C(=O)C(C(=O)NCCOCC(F)(F)F)C2=O)c1ccc2c(c1)C(=O)C(C(=O)NCCOCC(F)(F)F)C2=O]

The title compound was prepared in a similar manner to the synthesis of Example 18 from 5,5′-carbonylbis(1H-indene-1,3(2H)-dione) (150 mg, 0.47 mmol) and 1,1,1-trifluoro-2-(2-isocyanatoethoxy)ethane (CAS number 1341094-58-3, 199 mg, 1.2 mmol) to obtain a dark solid bis sodium salt (15 mg, 5%). LC/MS (ESI+): R_{f}=5.60 min, (M+H)⁺=657.11; HRMS (ESI−): calculated for C₂₉H₂₂F₆N₂O₉ m/z [M−H]⁻: 655.1157, observed 655.1143; ¹H NMR δ 8.48-8.72 (m, 2H), 7.66-7.88 (m, 2H), 7.53 (s, 2H), 7.34-7.46 (m, 2H), 3.92-4.17 (m, 4H), 3.48-3.69 (m, 4H).

### Example 68—Synthesis of 5,5′-carbonylbis(n-cyclobutyl-1,3-dioxo-2,3-dihydro-1h-indene-2-carboxamide)

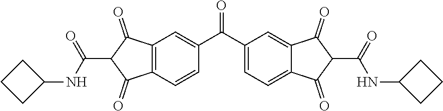 [SMILES: O=C(c1ccc2c(c1)C(=O)C(C(=O)NC1CCC1)C2=O)c1ccc2c(c1)C(=O)C(C(=O)NC1CCC1)C2=O]

The title compound was prepared in a similar manner to the synthesis of Example 18 from 5,5′-carbonylbis(1H-indene-1,3(2H)-dione) (200 mg, 0.63 mmol) and cyclobutyl isocyanate (CAS number 5811-25-6, 205 μl, 1.89 mmol,) to obtain a dark solid bis sodium salt (142 mgs, 44%). LCMS R_{f}=5.22; HRMS (ESI−): calculated for C₂₉H₂₃N₂O₇ m/z [M−H]⁻: 511.1511, observed 511.1494; ¹H NMR δ: 8.58-8.77 (m, 2H), 7.78-7.86 (m, 2H), 7.56 (s, 2H), 7.45 (d, J=7.6 Hz, 2H), 4.28-4.46 (m, 2H), 2.11-2.29 (m, 4H), 1.74-1.94 (m, 4H), 1.50-1.71 (m, 4H).

### Example 69—Synthesis of 5,5′-sulfonylbis(n-(2-methoxyphenyl)-1,3-dioxo-2,3-dihydro-1h-indene-2-carboxamide)

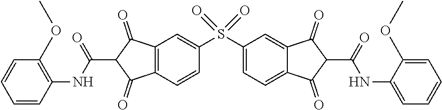 [SMILES: COc1ccccc1NC(=O)C1C(=O)c2ccc(S(=O)(=O)c3ccc4c(c3)C(=O)C(C(=O)Nc3ccccc3OC)C4=O)cc2C1=O]

The title compound was prepared in a similar manner to the synthesis of Example 33 from 5,5′-sulfonylbis(1H-indene-1,3(2H)-dione) (150 mg, 0.42 mmol) and 2-methoxyphenyl isocyanate (CAS Number: 700-87-8, 291 μl, 1.69 mmol) to obtain a dark solid bis sodium salt (109 mg, 37%). LCMS R_{f}=6.39; HRMS (ESI−): calculated for C₃₄H₂₃N₂O₁₀S m/z [M−H]⁻: 651.1079, observed 651.1061; ¹H NMR δ: 10.79-10.88 (m, 2H), 8.38-8.54 (m, 2H), 8.01-8.15 (m, 2H), 7.73 (s, 2H), 7.44-7.60 (m, 2H), 6.94-6.97 (m, 2H), 6.74-6.92 (m, 4H), 3.84 (s, 6H).

### Example 70—Synthesis of 5,5′-sulfonylbis(1,3-dioxo-n-(m-tolyl)-2,3-dihydro-1h-indene-2-carboxamide)

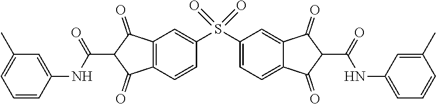 [SMILES: Cc1cccc(NC(=O)C2C(=O)c3ccc(S(=O)(=O)c4ccc5c(c4)C(=O)C(C(=O)Nc4cccc(C)c4)C5=O)cc3C2=O)c1]

The title compound was prepared in a similar manner to the synthesis of Example 33 from 5,5′-sulfonylbis(1H-indene-1,3(2H)-dione) (150 mg, 0.42 mmol) and m-tolyl isocyanate (CAS number 621-29-4; 233 μl, 3.72 mmol) to obtain a dark solid bis sodium salt (78 mg, 28%) LCMS R_{f}=4.10; HRMS (ESI−): calculated for C₃₄H₂₃N₂O₈S m/z [M−H]⁻: 619.1181, observed 619.1160; ¹H NMR δ: 10.59 (s, 2H), 8.02-8.20 (m, 2H), 7.68-7.83 (m, 2H), 7.49-7.65 (m, 2H), 7.38 (s, 4H), 7.01-7.20 (m, 2H), 6.61-6.86 (m, 2H), 2.23 (s, 6H).

### Example 71—Synthesis of 5,5′-carbonylbis(n-cyclopentyl-1,3-dioxo-2,3-dihydro-1h-indene-2-carboxamide)

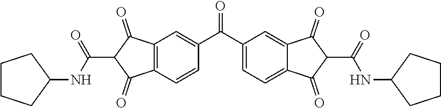 [SMILES: O=C(c1ccc2c(c1)C(=O)C(C(=O)NC1CCCC1)C2=O)c1ccc2c(c1)C(=O)C(C(=O)NC1CCCC1)C2=O]

The title compound was prepared in a similar manner to the synthesis of Example 18 from 5,5′-carbonylbis(1H-indene-1,3(2H)-dione) (200 mg, 0.62 mmol) and cylcopentyl isocyanate (CAS number: 4747-71-1, 272.1 μl, 2.26 mmol) to obtain a dark solid bis sodium salt (112, 30%) yield. LCMS R_{f}=5.36; HRMS (ESI−): calculated for C₃₁H₂₇N₂O₇ m/z [M−H]⁻: 539.1824, observed 539.1811; ¹H NMR δ: 8.45-8.61 (m, 2H), 7.76-7.84 (m, 2H), 7.55 (s, 2H), 7.41-7.48 (m, 2H), 4.03-4.27 (m, 2H), 1.74-1.93 (m, 4H), 1.45-1.73 (m, 6H), 1.27-1.44 (m, 4H).

### Example 72—Synthesis of 5,5′-carbonylbis(1,3-dioxo-n-(tetrahydro-2h-pyran-4-yl)-2,3-dihydro-1h-indene-2-carboxamide)

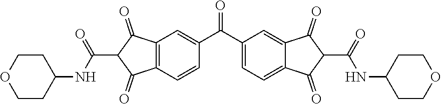 [SMILES: O=C(c1ccc2c(c1)C(=O)C(C(=O)NC1CCOCC1)C2=O)c1ccc2c(c1)C(=O)C(C(=O)NC1CCOCC1)C2=O]

The title compound was prepared in a similar manner to the synthesis of Example 18 from 5,5′-carbonylbis(1H-indene-1,3(2H)-dione) (100 mg, 0.31 mmol) and 4-tetrahydropyranyl isocyanate CAS number 53035-92-0, 236 μl, 1.26 mmol) to obtain a dark solid bis sodium salt (80 mg, 32%). LCMS R_{f}=5.36; HRMS (ESI−): calculated for C₃₁H₂₇N₂O₉ m/z [M−H]⁻: 571.1722, observed 571.1706; ¹H NMR δ: 8.51-8.71 (m, 2H), 7.80 (dd, J=7.5, 1.6 Hz, 2H), 7.54 (d, J=1.6 Hz, 2H), 7.44 (d, J=7.5 Hz, 2H), 3.39-3.92 (m, 8H), 3.08-3.24 (m, 2H), 1.37-1.96 (m, 8H).

### Example 73—Synthesis of 5,5′-sulfonylbis(n-(2-methoxyethyl)-1,3-dioxo-2,3-dihydro-1h-indene-2-carboxamide)

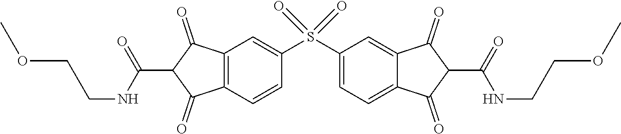

The title compound was prepared in a similar manner to the synthesis of Example 33, except the sodium salt formation was omitted, from 5,5′-sulfonylbis(1H-indene-1,3(2H)-dione) (100 mg, 0.28 mmol) and methoxyethyl isocyanate (CAS number 42170-95-6, 120.1 μl, 1.13 mmol) to obtain a dark solid (59 mg, 57%). LCMS R_{f}=4.10; HRMS (ESI−): calculated for C₂₆H₂₃N₂O₁₀S m/z [M−H]⁻: 555.1079, observed 555.1071; ¹H NMR (CDCl₃) δ: 8.09-8.28 (m, 2H), 7.77 (d, J=7.7 Hz, 2H), 7.37-7.52 (m, 2H), 3.48-3.73 (m, 8H), 3.41 (s, 6H).

### Example 74—Synthesis of 5,5′-carbonylbis(n-(2-methoxyethyl)-1,3-dioxo-2,3-dihydro-1h-indene-2-carboxamide)

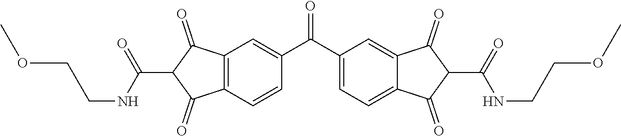

The title compound was prepared in a similar manner to the synthesis of Example 18 except the sodium salt formation was omitted, from 5,5′-carbonylbis(1H-indene-1,3(2H)-dione) (200 mg, 0.63 mmol) and methoxyethyl isocyanate (CAS number 42170-95-6, 200.1 μl, 1.89 mmol) to provide the title compound (64 mg, 18%). LCMS R_{f}=5.04; HRMS (ESI−): calculated for C₂₇H₂₃N₂O₉ m/z [M−H]⁻: 519.1409, observed 519.1429; ¹H NMR δ: 8.63-9.08 (m, 2H), 7.97 (br d, J=7.7 Hz, 2H), 7.80 (br s, 2H), 7.64-7.73 (m, 2H), 3.61-3.72 (m, 4H), 3.52-3.60 (m, 4H), 3.41 (s, 6H).

### Example 75—Synthesis of 1h,1′h,2h,2′h,3h,3′h-[5,5′-biindene]-1,1′,3,3′-tetrone

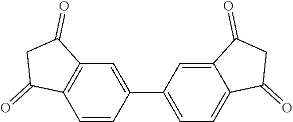 [SMILES: O=C1CC(=O)c2cc(-c3ccc4c(c3)C(=O)CC4=O)ccc21]

A stirred mixture of 4,4′-biphthalic anhydride (CAS number: 2420-87-3, 6.0 g, 20.39 mmol), anhydrous DMF 12.0 ml, isopropyl acetoacetate (CAS number: 542-08-5, 7.11 ml, 49.0 mmol), and acetic anhydride (18.6 ml, 255 mmol) was heated to 80° C. for 10 minutes. Triethylamine (8.5 ml 61.0 mmol) was then added dropwise. This dark solution was heated at 80° C. for 36 hours and checked by LCMS for consumption of the starting material. The flask was removed from the heating mantle and allowed to cool to room temperature. A large excess of 2N HCl 100 ml was added which caused a yellow solid to precipitate. The mixture was gently refluxed for 12 hr. After cooling to room temperature, the reaction mixture was filtered and the cake was washed with 2N HCl 100 ml and water 100 ml. The dried solid was milled to a fine solid and triturated with a minimal amount acetonitrile and ethyl acetate 3:1. There was obtained 5.82 g of the titled compound as a brown solid corresponding to a 98% yield. LCMS (ESI+): Rf=4.16; HRMS (ESI−): calculated for C₁₈H₉O₄ m/z [M−H]⁻: 289.0506, observed 289.0510; ¹H NMR δ: 8.39 (dd, J=8.0, 1.7 Hz, 2H), 8.32 (d, J=1.7 Hz, 2H), 8.05 (d, J=8.0 Hz, 2H), 3.43-3.56 (s, 2H).

### Example 76—Synthesis of N-(cyclopropylmethyl)-5-{2-[(cyclopropylmethyl) carbamoyl]-1,3-dioxo-2,3-dihydro-1H-indene-5-carbonyl}-1,3-dioxo-2,3-dihydro-1H-indene-2-carboxamide

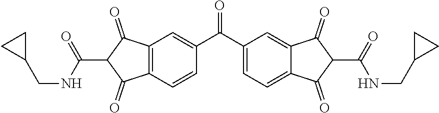 [SMILES: O=C(c1ccc2c(c1)C(=O)C(C(=O)NCC1CC1)C2=O)c1ccc2c(c1)C(=O)C(C(=O)NCC1CC1)C2=O]

The title compound was prepared in a similar manner to the synthesis of Example 18 from 5,5′-carbonylbis(1H-indene-1,3(2H)-dione) (100 mg, 0.31 mmol) and (isocyanatomethyl)cyclopropane (CAS number 25694-89-7, 114.5 μl, 1.26 mmol,) to obtain a dark solid bis sodium salt (64 mg, 37%). LCMS R_{f}=4.63; HRMS (ESI−): calculated for C₂₉H₂₃N₂O₇ m/z [M−H]⁻: 511.1511, observed 511.1526; ¹H NMR δ: 8.50-8.62 (m, 2H), 7.75-7.88 (m, 2H), 7.55 (s, 2H), 7.36-7.50 (m, 2H), 3.09 (m, 4H), 0.81-1.08 (m, 2H), 0.30-0.58 (m, 4H), 0.07-0.28 (m, 4H).

### Example 77—Synthesis of N-butyl-5-[2-(butylcarbamoyl)-1,3-dioxo-2,3-dihydro-1H-indene-5-carbonyl]-1,3-dioxo-2,3-dihydro-1H-indene-2-carboxamide

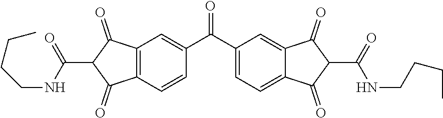 [SMILES: CCCCNC(=O)C1C(=O)c2ccc(C(=O)c3ccc4c(c3)C(=O)C(C(=O)NCCCC)C4=O)cc2C1=O]

The title compound was prepared in a similar manner to the synthesis of Example 18 from 5,5′-carbonylbis(1H-indene-1,3(2H)-dione) (100 mg, 0.31 mmol) and butyl isocyanate (CAS number: 111-36-4, 116 μl, 1.24 mmol) to obtain a dark solid bis sodium salt (76 mg, 44%). LCMS R_{f}=6.62; HRMS (ESI−): calculated for C₂₉H₂₇N₂O₇ m/z [M−H]⁻: 515.1824, observed 515.1830; ¹H NMR δ: 8.37-8.63 (m, 2H), 7.72-7.94 (m, 2H), 7.51-7.60 (m, 2H), 7.35-7.51 (m, 2H), 3.02-3.25 (m, 4H), 1.14-1.65 (m, 8H), 0.88 (t, J=7.2 Hz, 3H).

### Example 78—Synthesis of N-(4-chlorophenyl)-5-{2-[(4-chlorophenyl)carbamoyl]-1,3-dioxo-2,3-dihydro-1H-indene-5-carbonyl}-1,3-dioxo-2,3-dihydro-1H-indene-2-carboxamide

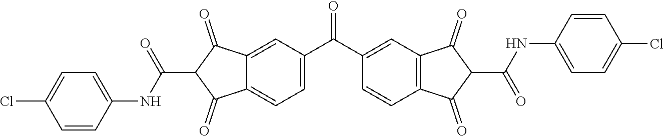 [SMILES: O=C(c1ccc2c(c1)C(=O)C(C(=O)Nc1ccc(Cl)cc1)C2=O)c1ccc2c(c1)C(=O)C(C(=O)Nc1ccc(Cl)cc1)C2=O]

The title compound was prepared in a similar manner to the synthesis of Example 18 from 5,5′-carbonylbis(1H-indene-1,3(2H)-dione) (100 mg, 0.31 mmol) and 4-chlorophenyl isocyanate (CAS number 104-12-1, 190.7 mg, 1.24 mmol) to obtain a dark solid bis sodium salt (136 mg, 65%). LCMS R_{f}=7.26; HRMS (ESI−): calculated for C₃₃H₁₇Cl₂N₂O₇ m/z [M−H]⁻: 623.0418, observed 623.0402; ¹H NMR δ: 10.87 (s, 2H), 7.86 (d, J=7.5 Hz, 2H), 7.59-7.71 (m, 6H), 7.54 (d, J=7.5 Hz, 2H), 7.27-7.32 (m, 4H).

### Example 79—Synthesis of N-(3-chlorophenyl)-5-{2-[(3-chlorophenyl)carbamoyl]-1,3-dioxo-2,3-dihydro-1H-indene-5-carbonyl}-1,3-dioxo-2,3-dihydro-1H-indene-2-carboxamide

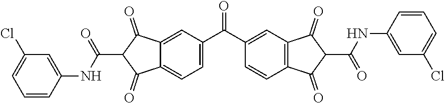 [SMILES: O=C(c1ccc2c(c1)C(=O)C(C(=O)Nc1cccc(Cl)c1)C2=O)c1ccc2c(c1)C(=O)C(C(=O)Nc1cccc(Cl)c1)C2=O]

The title compound was prepared in a similar manner to the synthesis of Example 18 from 5,5′-carbonylbis(1H-indene-1,3(2H)-dione) (100 mg, 0.31 mmol) and 3-chlorophenyl isocyanate (CAS number 2909-38-8, 190.7 mg, 1.24 mmol) to obtain a dark solid bis sodium salt (109 mg, 52%). LCMS R_{f}=7.15; HRMS (ESI−): calculated for C₃₅H₃₃F₆O₆ m/z [M−H]⁻: 663.2187, observed 663.2179; ¹H NMR δ: 10.83-10.87 (m, 2H), 8.04 (t, J=2.0 Hz, 2H), 7.85-7.90 (m, 2H), 7.65 (s, 2H), 7.55 (d, J=7.5 Hz, 2H), 7.22-7.32 (m, 4H), 6.99-6.95 (m, 2H).

### Example 80—Synthesis of N-(2,4-dichlorophenyl)-5-{2-[(2,4-dichlorophenyl) carbamoyl]-1,3-dioxo-2,3-dihydro-1H-indene-5-carbonyl}-1,3-dioxo-2,3-dihydro-1H-indene-2-carboxamide

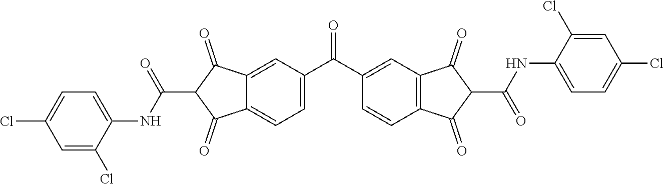 [SMILES: O=C(c1ccc2c(c1)C(=O)C(C(=O)Nc1ccc(Cl)cc1Cl)C2=O)c1ccc2c(c1)C(=O)C(C(=O)Nc1ccc(Cl)cc1Cl)C2=O]

The title compound was prepared as the bis-sodium salt in a similar manner to the synthesis of Example 18 from 5,5′-carbonylbis(1H-indene-1,3(2H)-dione) (100 mg, 0.31 mmol) and 2,4-dichlorophenyl isocyanate (CAS number 2612-57-9, 233.3 mg, 1.24 mmol) to obtain a dark solid bis sodium salt (103 mg, 45%). LCMS R_{f}=6.37; HRMS (ESI−): calculated for C₃₃H₁₅Cl₄N₂O₇ m/z [M−H]⁻: 690.9639, observed 690.9635; ¹H NMR δ: 11.18 (s, 1H), 8.50-8.73 (m, 2H), 7.81-7.96 (m, 2H), 7.67 (s, 3H), 7.49-7.63 (m, 4H), 7.20-7.36 (m, 2H)

### Example 81—Synthesis of N-(2-methylphenyl)-5-{2-[(2-methylphenyl)carbamoyl]-1,3-dioxo-2,3-dihydro-1H-indene-5-carbonyl}-1,3-dioxo-2,3-dihydro-1H-indene-2-carboxamide

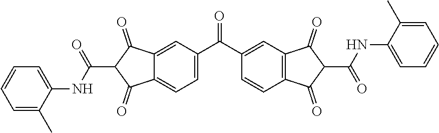 [SMILES: Cc1ccccc1NC(=O)C1C(=O)c2ccc(C(=O)c3ccc4c(c3)C(=O)C(C(=O)Nc3ccccc3C)C4=O)cc2C1=O]

The title compound was prepared in a similar manner to the synthesis of Example 18 from 5,5′-carbonylbis(1H-indene-1,3(2H)-dione) (100 mg, 0.31 mmol) and 2-methyphenyl isocyanate (CAS number 2612-57-9, 165 mg, 1.24 mmol) to obtain a dark solid bis sodium salt (105 mg, 54%). LCMS R_{f}=7.21; HRMS (ESI−): calculated for C₃₅H₂₃N₂O₇ m/z [M−H]⁻: 583.1511, observed 583.1496; ¹H NMR δ: 10.65 (m, 2H), 8.28-8.48 (m, 2H), 7.83-7.95 (m, 2H), 7.60-7.70 (m, 2H), 7.50-7.58 (m, 2H), 6.79-7.25 (m, 6H), 2.32 (s, 6H).

### Example 82—Synthesis of N-[2-(3-chlorophenyl)ethyl]-5-(2-{[2-(3-chiorophenyl)ethyl]carbamoyl}-1,3-dioxo-2,3-dihydro-1H-indene-5-carbonyl)-1,3-dioxo-2,3-dihydro-1H-indene-2-carboxamide

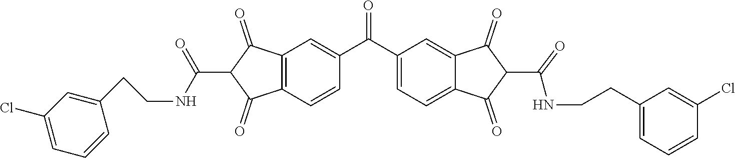 [SMILES: O=C(c1ccc2c(c1)C(=O)C(C(=O)NCCc1cccc(Cl)c1)C2=O)c1ccc2c(c1)C(=O)C(C(=O)NCCc1cccc(Cl)c1)C2=O]

The title compound was prepared in a similar manner to the synthesis of Example 18 from 5,5′-carbonylbis(1H-indene-1,3(2H)-dione) (100 mg, 0.31 mmol) and 3-chlorophenethyl isocyanate (CAS number 62334-11-6, 279.9 μl, 1.24 mmol) to obtain a dark solid bis sodium salt (116 mg, 52%). LCMS R_{f}=5.38; HRMS (ESI−): calculated for C₃₇H₂₅Cl₂N₂O₇ m/z [M−H]⁻: 679.1044, observed 679.1063; ¹H NMR δ: 8.76 (t, J=5.9 Hz, 2H), 7.67-7.89 (m, 2H), 7.55 (s, 4H), 7.45 (d, J=7.6 Hz, 2H), 6.29-6.38 (m, 2H), 6.22 (d, J=2.9 Hz, 2H), 4.34-4.48 (m, 4H).

### Example 83—Synthesis of N-[2-(4-fluorophenyl)ethyl]-5-[(2-{[2-(4-fluorophenyl)ethyl]carbamoyl}-1,3-dioxo-2,3-dihydro-1H-inden-5-yl)sulfonyl]-1,3-dioxo-2,3-dihydro-1H-indene-2-carboxamide

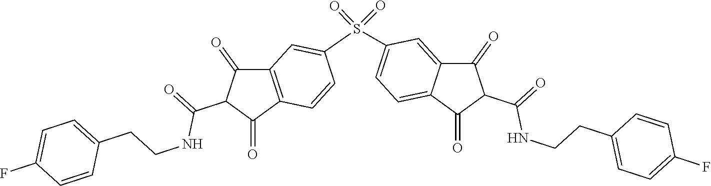 [SMILES: O=C(NCCc1ccc(F)cc1)C1C(=O)c2ccc(S(=O)(=O)c3ccc4c(c3)C(=O)C(C(=O)NCCc3ccc(F)cc3)C4=O)cc2C1=O]

The title compound was prepared in a similar manner to the synthesis of Example 33 from 5,5′-sulfonylbis(1H-indene-1,3(2H)-dione) (100 mg, 0.28 mmol) and 4-fluorophenethyl isocyanate (CAS number 65535-53-7, 211.1 μl, 1.13 mmol) to obtain a dark solid bis sodium salt (132 mg, 64%) yield. LCMS R_{f}=4.10; HRMS (ESI−): calculated for C₃₆H₂₅F₂N₂O₈S m/z [M−H]⁻: 683.1305, observed 683.1322; ¹H NMR δ: 8.32-8.51 (m, 2H), 7.99-8.06 (m, 2H), 7.62 (s, 2H), 7.46 (d, J=7.6 Hz, 2H), 7.18-7.29 (m, 4H), 7.01-7.15 (m, 4H), 3.37-3.44 (m, 4H), 2.66-2.78 (m, 4H).

### Example 84—Synthesis of N-[2-(3-chlorophenyl)ethyl]-5-[(2-{[2-(3-chlorophenyl)ethyl]carbamoyl}-1,3-dioxo-2,3-dihydro-1H-inden-5-yl)sulfonyl]-1,3-dioxo-2,3-dihydro-1H-indene-2-carboxamide

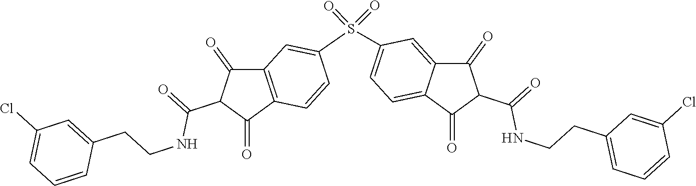 [SMILES: O=C(NCCc1cccc(Cl)c1)C1C(=O)c2ccc(S(=O)(=O)c3ccc4c(c3)C(=O)C(C(=O)NCCc3cccc(Cl)c3)C4=O)cc2C1=O]

The title compound was prepared in a similar manner to the synthesis of Example 33 from 5,5′-sulfonylbis(1H-indene-1,3(2H)-dione) (100 mg, 0.28 mmol) and 3-chlorophenethyl isocyanate (CAS number 62334-11-6, 254.2 μl, 1.13 mmol) to obtain a dark solid bis sodium salt (174 mg, 81%). LCMS R_{f}=4.10; HRMS (ESI−): calculated for C₃₆H₂₅Cl₂N₂O₈S m/z [M−H]⁻: 715.0714, observed 715.0725; ¹H NMR δ: 8.19-8.71 (m, 2H), 8.12 (s, 2H), 7.56-7.72 (m, 2H), 7.42-7.53 (m, 2H), 7.15-7.34 (m, 4H), 7.08 (s, 4H), 3.35-3.47 (m, 4H), 2.62-2.85 (m, 4H).

### Example 85—Synthesis of N-(2-methylphenyl)-5-({2-[(2-methylphenyl)carbamoyl]-1,3-dioxo-2,3-dihydro-1H-inden-5-yl}sulfonyl)-1,3-dioxo-2,3-dihydro-1H-indene-2-carboxamide

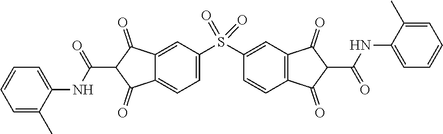 [SMILES: Cc1ccccc1NC(=O)C1C(=O)c2ccc(S(=O)(=O)c3ccc4c(c3)C(=O)C(C(=O)Nc3ccccc3C)C4=O)cc2C1=O]

The title compound was prepared in a similar manner to the synthesis of Example 33 from 5,5′-sulfonylbis(1H-indene-1,3(2H)-dione) (100 mg, 0.28 mmol) and o-tolyl isocyanate (CAS number 614-68-6, 161.4 μl, 1.13 mmol) to obtain a dark solid bis sodium salt (50 mg) corresponding to a 27% yield. LCMS R_{f}=4.10; HRMS (ESI−): calculated for C₃₄H₂₃N₂O₈S m/z [M−H]⁻: 619.1181, observed 619.1170; ¹H NMR δ: 10.59 (s, 2H), 8.29-8.38 (m, 2H), 8.05-8.14 (m, 2H), 7.75 (s, 2H), 7.53-7.59 (m, 2H), 7.03-7.16 (m, 4H), 6.81-6.87 (m, 2H), 2.30 (s, 6H).

### Example 86—Synthesis of N-(3-chlorophenyl)-5-({2-[(3-chlorophenyl)carbamoyl]-1,3-dioxo-2,3-dihydro-1H-inden-5-yl}sulfonyl)-1,3-dioxo-2,3-dihydro-1H-indene-2-carboxamide

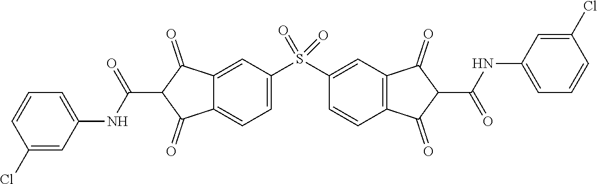 [SMILES: O=C(Nc1cccc(Cl)c1)C1C(=O)c2ccc(S(=O)(=O)c3ccc4c(c3)C(=O)C(C(=O)Nc3cccc(Cl)c3)C4=O)cc2C1=O]

The title compound was prepared in a similar manner to the synthesis of Example 33 from 5,5′-sulfonylbis(1H-indene-1,3(2H)-dione) (100 mg, 0.28 mmol) and 3-chlorophenyl isocyanate (CAS number 2909-38-8, 173.3 mg, 1.13 mmol) to obtain a dark solid bis sodium salt (123 mg, 62%). LCMS R_{f}=4.10; HRMS (ESI−): calculated for C₃₂H₁₇Cl₂N₂O₈S m/z [M−H]⁻: 659.0088, observed 659.0094; ¹H NMR δ: 10.79 (s, 2H), 8.05-8.29 (m, 2H), 7.88-8.05 (m, 2H), 7.68-7.87 (m, 2H), 7.43-7.67 (m, 2H), 7.18-7.41 (m, 4H), 6.93-7.01 (m, 2H).

### Example 87—Synthesis of N-(4-chlorophenyl)-5-({2-[(4-chlorophenyl)carbamoyl]-1,3-dioxo-2,3-dihydro-1H-inden-5-yl}sulfonyl)-1,3-dioxo-2,3-dihydro-1H-indene-2-carboxamide

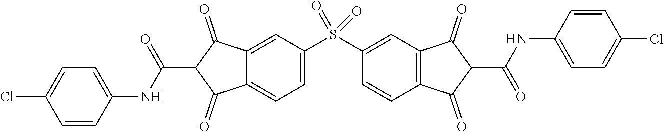 [SMILES: O=C(Nc1ccc(Cl)cc1)C1C(=O)c2ccc(S(=O)(=O)c3ccc4c(c3)C(=O)C(C(=O)Nc3ccc(Cl)cc3)C4=O)cc2C1=O]

The title compound was prepared in a similar manner to the synthesis of Example 33 from 5,5′-sulfonylbis(1H-indene-1,3(2H)-dione) (100 mg, 0.23 mmol) and 4-chlorophenyl isocyanate (CAS number 104-12-1, 173.3 mg, 1.13 mmol) to obtain a dark solid bis sodium salt (107 mg, 54%). LCMS R_{f}=4.10; HRMS (ESI−): calculated for C₃₂H₁₇Cl₂N₂O₈S m/z [M−H]⁻: 659.0088, observed 659.0096; ¹H NMR δ: 10.77 (s, 2H), 8.10 (dd, J=7.6, 1.8 Hz, 2H), 7.74 (d, J=1.7 Hz, 2H), 7.51-7.68 (m, 6H), 7.28 (d, J=8.8 Hz, 4H).

### Example 88—Synthesis of N-(3-methoxypropyl)-5-{2-[(3-methoxypropyl)carbamoyl]-1,3-dioxo-2,3-dihydro-1H-indene-5-carbonyl}-1,3-dioxo-2,3-dihydro-1H-indene-2-carboxamide

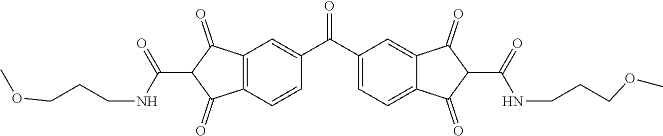

The title compound was prepared in a similar manner to the synthesis of Example 18 from 5,5′-carbonylbis(1H-indene-1,3(2H)-dione) (150 mg, 0.94 mmol) and 3-methoxypropyl isocyanate (CAS number 7019-13-8, 140 μL, 1.2 mmol) to afford the title compound (12 mg, 5%). LC/MS (ESI+): R_{f}=5.63 min, (M+H)⁺=549.21; HRMS (ESI−): calculated for C₂₉H₂₈N₂O₉ m/z [M−H]⁻: 547.1722, observed 547.1719; ¹H NMR (CDCl₃) δ 8.42-8.56 (m, 2H), 7.95-8.07 (m, 4H), 7.70-7.84 (m, 2H), 3.51-3.66 (m, 8H), 3.45 (d, J=5.86 Hz, 6H), 1.87-1.99 (m, 4H).

### Example 89—Synthesis of 5-[(1,3-dioxo-2-{[(oxolan-2-yl)methyl]carbamoyl}-2,3-dihydro-1H-inden-5-yl)sulfonyl]-1,3-dioxo-N-[(oxolan-2-yl)methyl]-2,3-dihydro-1H-indene-2-carboxamide

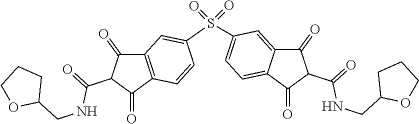 [SMILES: O=C(NCC1CCCO1)C1C(=O)c2ccc(S(=O)(=O)c3ccc4c(c3)C(=O)C(C(=O)NCC3CCCO3)C4=O)cc2C1=O]

The title compound was prepared in a similar manner to the synthesis of Example 33 from 5,5′-sulfonylbis(1H-indene-1,3(2H)-dione) (150 mg, 0.43 mmol) and 2-(isocyanatomethyl)oxolane (CAS number 51998-05-1118 μL, 0.9 mmol). The resulting material was then purified using a C18 reverse phase column with acetonitrile/water and 0.1% ammonium hydroxide modifier to afford the title compound as the bis ammonium salt (64 mg, 25%). LC/MS (ESI+): R_{f}=6.11 min, (M+H)⁺=609.72; HRMS (ESI−): calculated for C₃₀H₂₈N₂O₁₀S m/z [M−H]⁻: 607.1392, observed 607.1389; ¹H NMR (D₂O) δ 8.03 (d, J=6.9 Hz, 2H), 7.81 (s, 2H), 7.48 (d, J=7.8 Hz, 2H), 3.88-4.14 (m, 2H), 3.57-3.90 (m, 4H), 3.05-3.38 (m, 4H), 1.68-2.23 (m, 6H), 1.04-1.65 (m, 2H).

### Example 90—Synthesis of N-(3-methoxypropyl)-5-({2-[(3-methoxypropyl)carbamoyl]-1,3-dioxo-2,3-dihydro-1H-inden-5-yl}sulfonyl)-1,3-dioxo-2,3-dihydro-1H-indene-2-carboxamide

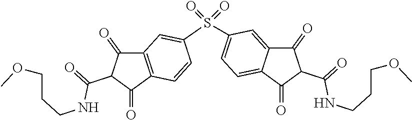

The title compound was prepared in a similar manner to the synthesis of Example 33 from 5,5′-sulfonylbis(1H-indene-1,3(2H)-dione) (150 mg, 0.43 mmol) and 3-methoxypropyl isocyanate (CAS number 7019-13-8, 105 μL, 0.9 mmol). The resulting material was then purified using a C18 reverse phase column with acetonitrile/water and 0.1% ammonium hydroxide modifier to afford the title compound as the bis ammonium salt (21 mg, 9%). LC/MS (ESI+): R_{f}=5.54 min, (M+H)⁺=585.78; HRMS (ESI−): calculated for C₂₈H₂₈N₂O₁₀S m/z [M−H]⁻: 583.1392, observed 583.1404; ¹H NMR (D₂O) δ 7.93-8.13 (m, 2H), 7.81 (s, 2H), 7.38-7.59 (m, 2H), 3.44 (t, J=6.15 Hz, 4H), 3.10-3.28 (m, 6H), 1.54-1.90 (m, 2H).

### Example 91—Synthesis of N-(4-methylphenyl)-5-{2-[(4-methylphenyl)carbamoyl]-1,3-dioxo-2,3-dihydro-1H-indene-5-carbonyl}-1,3-dioxo-2,3-dihydro-1H-indene-2-carboxamide

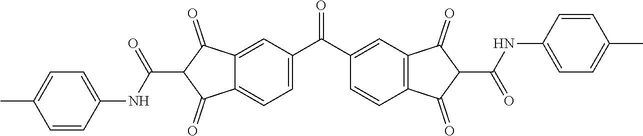

The title compound was prepared in a similar manner to the synthesis of Example 18 from 5,5′-carbonylbis(1H-indene-1,3(2H)-dione) (100 mg, 0.31 mmol) and p-tolyl isocyanate (CAS number 622-58-2, 177.5 μl, 1.26 mmol) to obtain a dark solid bis-sodium salt (126 mg, 64%). LCMS R_{f}=7.52; HRMS (ESI−): calculated for C₃₅H₂₃N₂O₇ m/z [M−H]⁻: 583.1511, observed 583.1499; ¹H NMR δ: 10.61-10.70 (m, 2H), 7.82-7.90 (m, 2H), 7.64 (s, 2H), 7.46-7.57 (m, 6H), 6.99-7.09 (m, 4H), 2.21 (s, 6H).

### Example 92—Synthesis of N-(4-methylphenyl)-5-({2-[(4-methylphenyl)carbamoyl]-1,3-dioxo-2,3-dihydro-1H-inden-5-yl}sulfonyl)-1,3-dioxo-2,3-dihydro-1H-indene-2-carboxamide

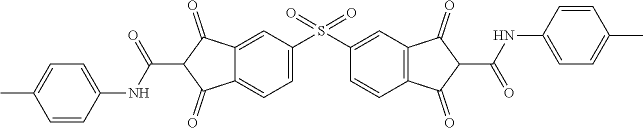

The title compound was prepared in a similar manner to the synthesis of Example 33 from 5,5′-sulfonylbis(1H-indene-1,3(2H)-dione) (100 mg, 0.28 mmol) and 4-tolyl isocyanate (CAS number 622-58-2) (161.4 μl, 1.13 mmol) to obtain a dark solid bis-sodium salt (115 mg, 61%). LCMS R_{f}=4.10; HRMS (ESI−): calculated for C₃₄H₂₃N₂O₈S m/z [M−H]⁻: 619.1181, observed 619.1165; ¹H NMR δ: 10.60 (s, 2H), 8.10 (dd, J=7.6, 1.7 Hz, 2H), 7.72 (d, J=1.7 Hz, 2H), 7.54 (d, J=7.6 Hz, 2H), 7.47 (d, J=8.3 Hz, 4H), 7.04 (d, J=8.2 Hz, 4H), 2.22 (s, 6H).

### Example 93—Synthesis of N-benzyl-5-{[2-(benzylcarbamoyl)-1,3-dioxo-2,3-dihydro-1H-inden-5-yl]sulfonyl}-1,3-dioxo-2,3-dihydro-1H-indene-2-carboxamide

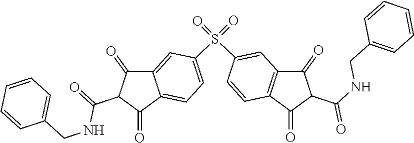 [SMILES: O=C(NCc1ccccc1)C1C(=O)c2ccc(S(=O)(=O)c3ccc4c(c3)C(=O)C(C(=O)NCc3ccccc3)C4=O)cc2C1=O]

The title compound was prepared in a similar manner to the synthesis of Example 33 from 5,5′-sulfonylbis(1H-indene-1,3(2H)-dione) (100 mg, 0.28 mmol) and 2-benzyl isocyanate (CAS number 3173-56-6, 162.2 μl, 1.13 mmol) to obtain a dark solid bis-sodium salt (122 mg, 65%) LCMS R_{f}=4.10; HRMS (ESI−): calculated for C₃₄H₂₃N₂O₈S m/z [M−H]⁻: 619.1181, observed 619.1169; ¹H NMRS δ: 8.79 (s, 2H), 8.00-8.09 (m, 2H), 7.64 (d, J=1.7 Hz, 2H), 7.48 (d, J=7.6 Hz, 2H), 7.16-7.35 (m, 10H), 4.40 (br d, J=6.0 Hz, 4H).

### Example 94—Synthesis of N-(2,4-dimethoxyphenyl)-5-({2-[(2,4-dimethoxyphenyl)carbamoyl]-1,3-dioxo-2,3-dihydro-1H-inden-5-yl}sulfonyl)-1,3-dioxo-2,3-dihydro-1H-indene-2-carboxamide

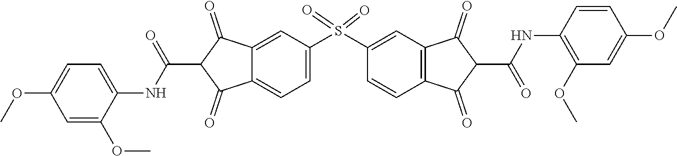

The title compound was prepared in a similar manner to the synthesis of Example 33 from 5,5′-sulfonylbis(1H-indene-1,3(2H)-dione) (150 mg, 0.43 mmol) and 2,4-dimethoxyphenyl isocyanate (CAS number 84370-87-6, 228 μL, 0.9 mmol). The resulting material was then purified using a C18 reverse phase column with acetonitrile/water and 0.1% ammonium hydroxide modifier. Fractions were concentrated and washed with methylene chloride and acetonitrile to afford the title compound as the bis ammonium salt (72 mg, 24%). LC/MS (ESI+): R_{f}=7.28 min, (M+H)⁺=713.43; HRMS (ESI−): calculated for C₃₆H₂₈N₂O₁₂S m/z [M−H]⁻: 711.1290, observed 711.1278; ¹H NMR δ 10.70 (s, 1H), 8.35 (d, J=5.4 Hz, 2H), 8.05 (d, J=6 Hz, 2H), 7.72 (s, 2H), 7.41 (d, J=6.3 Hz, 2H), 6.60 (s, 2H), 6.39 (d, J=4.8 Hz, 2H), 3.84 (s, 6H), 3.72 (s, 6H).

### Example 95—Synthesis of N-(4-iodophenyl)-5-({2-[(4-iodophenyl)carbamoyl]-1,3-dioxo-2,3-dihydro-1H-inden-5-yl}sulfonyl)-1,3-dioxo-2,3-dihydro-1H-indene-2-carboxamide

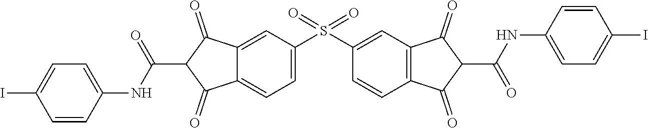 [SMILES: O=C(Nc1ccc(I)cc1)C1C(=O)c2ccc(S(=O)(=O)c3ccc4c(c3)C(=O)C(C(=O)Nc3ccc(I)cc3)C4=O)cc2C1=O]

The title compound was prepared in a similar manner to the synthesis of Example 33 from 5,5′-sulfonylbis(1H-indene-1,3(2H)-dione) (150 mg, 0.43 mmol) and 4-iodophenyl isocyanate (CAS number 15845-62-2, 289 mg, 0.9 mmol). The resulting material was washed with acetonitrile/water and 0.1% ammonium hydroxide modifier. The resulting solid was washed with methylene chloride and acetonitrile to afford the title compound as the bis ammonium salt (268 mg, 70%). HRMS (ESI−): calculated for C₃₆H₂₈N₂O₁₂S m/z [M−H]⁻: 842.8801, observed 842.8799; ¹H NMR δ 10.84 (s, 2H), 8.84 (s, 2H), 8.17 (d, J=6.9 Hz, 2H), 7.73 (s, 2H), 7.49-7.63 (m, 6H), 7.36-7.49 (d, J=3.9 Hz, 2H), 7.20-7.36 (m, 2H).

### Example 96—Synthesis of N-(2,4-dimethoxyphenyl)-5-{2-[(2,4-dimethoxyphenyl)carbamoyl]-1,3-dioxo-2,3-dihydro-1H-indene-5-carbonyl}-1,3-dioxo-2,3-dihydro-1H-indene-2-carboxamide

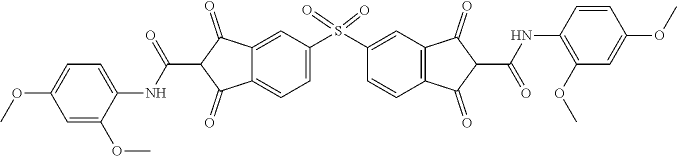

The title compound was prepared in a similar manner to the synthesis of Example 18 from 5,5′-carbonylbis(1H-indene-1,3(2H)-dione) (150 mg, 0.94 mmol) and 2,4-dimethoxyphenyl isocyanate (CAS number 84370-87-6, 211 mg, 1.2 mmol). The product was purified with acetonitrile/water and 0.1% ammonium hydroxide modifier to afford the title compound as the bis ammonium salt (12 mg, 4%). LC/MS (ESI+):_R_{f}=6.72 min, (M+H)⁺=677.59; HRMS (ESI−): calculated for C₃₇H₂₈N₂O₁₁ m/z [M−H]⁻: 675.1620, observed 675.1611; ¹H NMR δ 10.79 (s, 2H), 8.37 (d, J=4.5 Hz, 2H), 7.83 (d, J=4.2 Hz, 2H), 7.61 (s, 2H), 7.48 (d, J=3.9 Hz, 2H), 6.58 (s, 2H), 6.43 (d, J=3.9 Hz, 1H), 3.87 (s, 6H), 3.72 (s, 6H).

### Example 97—Synthesis of methyl 2-{5-[(2-{[2-(methoxycarbonyl)phenyl]carbamoyl}-1,3-dioxo-2,3-dihydro-1H-inden-5-yl)sulfonyl]-1,3-dioxo-2,3-dihydro-1H-indene-2-amido}benzoate

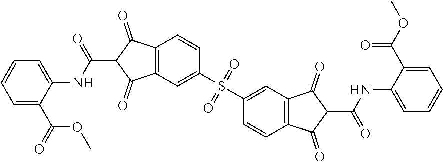 [SMILES: COC(=O)c1ccccc1NC(=O)C1C(=O)c2ccc(S(=O)(=O)c3ccc4c(c3)C(=O)C(C(=O)Nc3ccccc3C(=O)OC)C4=O)cc2C1=O]

The title compound was prepared in a similar manner to the synthesis of Example 33 from 5,5′-sulfonylbis(1H-indene-1,3(2H)-dione) (100 mg, 0.28 mmol) and 2-methoxycarbonylphenyl isocyanate (CAS number 1793-07-3, 200 mg, 1.13 mmol) to obtain a dark solid bis-sodium salt (131 mg, 62%). LCMS R_{f}=4.10; HRMS (ESI−): calculated for C₃₆H₂₂N₂NaO₁₂S m/z [M−H]⁻: 729.0797, observed 729.0807; ¹H NMR δ: 11.51 (s, 2H), 8.56 (s, 2H), 8.08-8.15 (m, 2H), 7.82 (d, J=1.8 Hz, 2H), 7.76 (d, J=1.7 Hz, 2H), 7.57 (d, J=7.6 Hz, 2H), 7.37-7.52 (m, 3H), 6.92-7.08 (m, 2H), 3.90 (s, 6H).

### Example 98—Synthesis of methyl 4-{5-[(2-{[4-(methoxycarbonyl)phenyl]carbamoyl}-1,3-dioxo-2,3-dihydro-1H-inden-5-yl)sulfonyl]-1,3-dioxo-2,3-dihydro-1H-indene-2-amido}benzoate

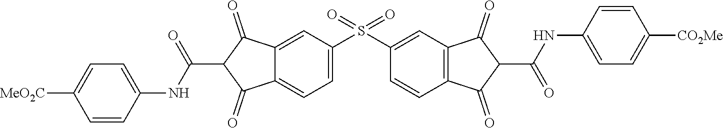 [SMILES: COC(=O)c1ccc(NC(=O)C2C(=O)c3ccc(S(=O)(=O)c4ccc5c(c4)C(=O)C(C(=O)Nc4ccc(C(=O)OC)cc4)C5=O)cc3C2=O)cc1]

The title compound was prepared in a similar manner to the synthesis of Example 33 from 5,5′-sulfonylbis(1H-indene-1,3(2H)-dione) (100 mg, 0.28 mmol) and 4-methoxycarbonylphenyl isocyanate (CAS number 23138-53-6, 200 mg, 1.13 mmol) to obtain a dark solid bis-sodium salt (132 mg, 62%). LCMS R_{f}=4.10; HRMS (ESI−): calculated for C₃₆H₂₃N₂O₁₂S m/z [M−H]⁻: 707.0977, observed 707.0968; ¹H NMR δ: 11.02 (s, 2H), 8.07-8.19 (m, 3H), 7.81-7.89 (m, 5H), 7.66-7.77 (m, 6H), 7.57 (d, J=7.6 Hz, 2H), 3.79 (s, 6H).

### Example 99—Synthesis of methyl 2-[5-(2-{[2-(methoxycarbonyl)phenyl]carbamoyl}-1,3-dioxo-2,3-dihydro-1H-indene-5-carbonyl)-1,3-dioxo-2,3-dihydro-1H-indene-2-amido]benzoate

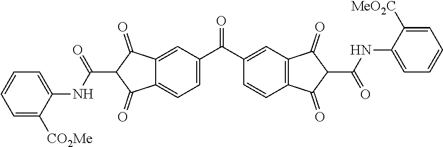 [SMILES: COC(=O)c1ccccc1NC(=O)C1C(=O)c2ccc(C(=O)c3ccc4c(c3)C(=O)C(C(=O)Nc3ccccc3C(=O)OC)C4=O)cc2C1=O]

The title compound was prepared in a similar manner to the synthesis of Example 18 from 5,5′-carbonylbis(1H-indene-1,3(2H)-dione) (100 mg, 0.31 mmol) and 2-methoxycarbonylphenyl isocyanate (CAS number 1793-07-3, 220 mg, 1.26 mmol) to obtain a dark solid bis-sodium salt (114 mg, 51%). LCMS R_{f}=6.38; HRMS (ESI−): calculated for C₃₇H₂₂N₂NaO₁₁ m/z [M−H]⁻: 693.1127, observed 693.1148; ¹H δ: 11.41-11.70 (m, 2H), 8.46-8.72 (m, 2H), 7.76-7.97 (m, 4H), 7.62-7.72 (m, 2H), 7.50-7.61 (m, 2H), 7.36-7.49 (m, 2H), 6.93-7.09 (m, 2H), 3.94 (s, 6H).

### Example 100—Synthesis of N-[2-(3,5-dimethoxyphenyl)ethyl]-5-(2-{[2-(3,5-dimethoxyphenyl)ethyl]carbamoyl}-1,3-dioxo-2,3-dihydro-1H-indene-5-carbonyl)-1,3-dioxo-2,3-dihydro-1H-indene-2-carboxamide

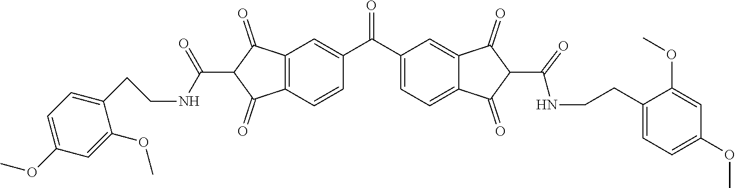

To a stirred solution of triphosgene (CAS number 32315-10-9, 426 mg, 1.4 mmole) in methylene chloride (20 ml), was added 3,5-dimethoxyphenethylamine (CAS number 3213-28-3, 500 μl, 2.9 mmol) dropwise. Triethylamine (599 μL, 1.9 mmol) was added dropwise and the solution was allowed to stir for 15 min. The resulting mixture was concentrated in vacuo to afford 1-(2-isocyanatoethyl)-3,5-dimethoxybenzene. This and 5,5′-carbonylbis(1H-indene-1,3(2H)-dione) (150 mg, 0.94 mmol) were reacted according to the procedure in Example 18 to afford 1-(2-isocyanatoethyl)-3,5-dimethoxybenzene (244 mg, 1.2 mmol). The product was purified with a C18 reverse phase column using acetonitrile/water and 0.1% ammonium hydroxide modifier to afford the title compound as the bis ammonium salt (27 mg, 11%). LC/MS (ESI+): R_{f}=6.17 min, (M+H)⁺=733.96; HRMS (ESI−): calculated for C₃₇H₂₈N₂O₁₁ m/z [M−H]⁻: 731.2246, observed 731.2266; ¹H NMR δ 8.76-9.11 (m, 2H), 8.29 (s, 2H), 7.87-8.10 (m, 2H), 7.66-7.89 (m, 4H), 6.51 (s, 4H), 6.31 (s, 2H), 3.75 (s, 12H), 2.77-2.94 (m, 4H).

### Example 101—Synthesis of N-(4-butylphenyl)-5-{2-[(4-butylphenyl)carbamoyl]-1,3-dioxo-2,3-dihydro-1H-indene-5-carbonyl}-1,3-dioxo-2,3-dihydro-1H-indene-2-carboxamide

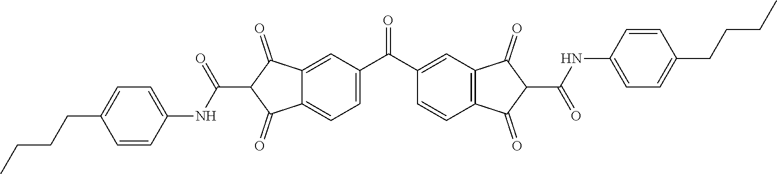 [SMILES: CCCCc1ccc(NC(=O)C2C(=O)c3ccc(C(=O)c4ccc5c(c4)C(=O)C(C(=O)Nc4ccc(CCCC)cc4)C5=O)cc3C2=O)cc1]

The title compound was prepared in a similar manner to the synthesis of Example 18 from 5,5′-carbonylbis(1H-indene-1,3(2H)-dione) (100 mg, 0.31 mmol) and 4-butylphenyl isocyanate (CAS number 69342-47-8, 215.7 μl, 1.26 mmol) to obtain a dark solid bis-sodium salt (132 mg, 60%). LCMS R_{f}=7.83,; HRMS (ESI−): calculated for C₄₁H₃₅N₂O₇ m/z [M−H]⁻: 667.2450, observed 667.2449; ¹H NMR δ: 10.71 (s, 2H), 7.85 (dd, J=7.5, 1.5 Hz, 2H), 7.63 (s, 2H), 7.52 (dd, J=7.8, 4.8 Hz, 6H), 7.06 (d, J=8.1 Hz, 4H), 3.15-3.19 (m, 4H), 1.51 (br t, J=7.5 Hz, 4H), 1.15-1.38 (m, 4H), 0.77-0.99 (m, 6H).

### Example 102—Synthesis of N-(4-butylphenyl)-5-[(2-{[(4-butylphenyl)amino](hydroxy)methyl}-1,3-dioxo-2,3-dihydro-1H-inden-5-yl)sulfonyl]-1,3-dioxo-2,3-dihydro-1H-indene-2-carboxamide

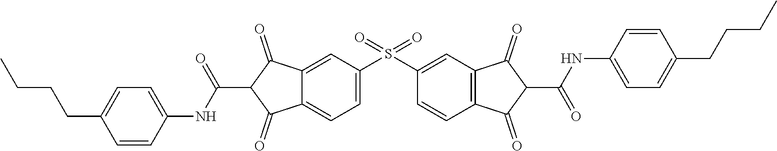 [SMILES: CCCCc1ccc(NC(=O)C2C(=O)c3ccc(S(=O)(=O)c4ccc5c(c4)C(=O)C(C(=O)Nc4ccc(CCCC)cc4)C5=O)cc3C2=O)cc1]

The title compound was prepared in a similar manner to the synthesis of Example 33 from 5,5′-sulfonylbis(1H-indene-1,3(2H)-dione) (100 mg, 0.28 mmol) and 4-butylphenyl isocyanate (CAS number 69342-47-8, 197 μl, 1.13 mmol) to obtain a dark solid bis sodium salt (85 mg, 38%). LCMS R_{f}=4.10; HRMS (ESI−): calculated for 1 C₄₀H₃₄N₂NaO₈S m/z [M−H]⁻: 725.193905, observed 725.194324; ¹H NMR δ: 10.60 (s, 2H), 8.09 (dd, J=7.6, 1.8 Hz, 4H), 7.71 (d, J=1.7 Hz, 4H), 7.40-7.60 (m, 8H), 7.05 (d, J=8.2 Hz, 4H), 2.48-2.55 (m 4H), 1.50-1.55 (m, 4H), 1.27 (br d, J=7.7 Hz, 6H).

### Example 103—Synthesis of N-(3-acetylphenyl)-5-{2-[(3-acetylphenyl)carbamoyl]-1,3-dioxo-2,3-dihydro-1H-indene-5-carbonyl}-1,3-dioxo-2,3-dihydro-1H-indene-2-carboxamide

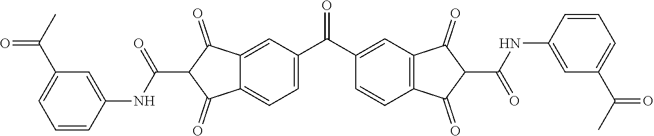 [SMILES: CC(=O)c1cccc(NC(=O)C2C(=O)c3ccc(C(=O)c4ccc5c(c4)C(=O)C(C(=O)Nc4cccc(C(C)=O)c4)C5=O)cc3C2=O)c1]

The title compound was prepared as the bis-sodium salt in a similar manner to the synthesis of Example 18 from 5,5′-carbonylbis(1H-indene-1,3(2H)-dione) (100 mg, 0.31 mmol) and 3-acetyllphenyl isocyanate (CAS number 23138-64-9, 234 μl, 1.26 mmol) to obtain a dark solid bis sodium salt (133 mg, 62%). LCMS R_{f}=6.42; HRMS (ESI−): calculated for C₃₇H₂₃N₂O₉ m/z [M−H]⁻: 639.1409, observed 639.1384; ¹H NMR δ: 10.94 (s, 2H), 8.19 (t, J=1.9 Hz, 2H), 7.85-7.89 (m, 4H), 7.65 (s, 2H), 7.52-7.57 (m, 4H), 7.36-7.42 (m, 2H), 2.56 (s, 6H).

### Example 104—Synthesis of N-[(furan-2-yl)methyl]-5-[(2-{[(furan-2-yl)methyl]carbamoyl}-1,3-dioxo-2,3-dihydro-1H-inden-5-yl)sulfonyl]-1,3-dioxo-2,3-dihydro-1H-indene-2-carboxamide

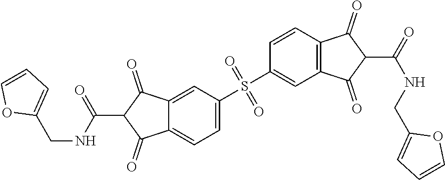 [SMILES: O=C(NCc1ccco1)C1C(=O)c2ccc(S(=O)(=O)c3ccc4c(c3)C(=O)C(C(=O)NCc3ccco3)C4=O)cc2C1=O]

The title compound was prepared as the bis-sodium salt in a similar manner to the synthesis of Example 33 from 5,5′-sulfonylbis(1H-indene-1,3(2H)-dione) (100 mg, 0.28 mmol)) and 2-(isocyanatomethyl)furan (CAS number 71189-15-6, 159.5 μl, 1.13 mmol) to obtain a dark solid bis sodium salt (126 mg, 70%). LCMS R_{f}=4.10; HRMS (ESI−): calculated for C₃₀H₁₉N₂O₁₀S m/z [M−H]⁻: 599.0766, observed 599.0750; ¹H NMR δ: 8.68 (br s, 2H), 8.04 (br d, J=8.1 Hz, 2H), 7.64 (br s, 2H), 7.42-7.58 (m, 4H), 6.36 (s, 2H), 6.20 (s, 2H), 4.38 (s, 4H).

### Example 105—Synthesis of N-(4-iodophenyl)-5-{2-[(4-iodophenyl)carbamoyl]-1,3-dioxo-2,3-dihydro-1H-indene-5-carbonyl}-1,3-dioxo-2,3-dihydro-1H-indene-2-carboxamide

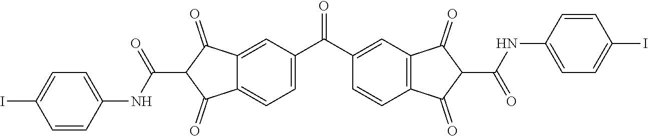 [SMILES: O=C(c1ccc2c(c1)C(=O)C(C(=O)Nc1ccc(I)cc1)C2=O)c1ccc2c(c1)C(=O)C(C(=O)Nc1ccc(I)cc1)C2=O]

The title compound was prepared in a similar manner to the synthesis of Example 18 from 5,5′-carbonylbis(1H-indene-1,3(2H)-dione) (150 mg, 0.94 mmol) and 4-iodophenyl isocyanate (CAS number 15845-62-2, 289 mg, 0.9 mmol). The resulting material was washed with acetonitrile/water and 0.1% ammonium hydroxide modifier. The resulting solid was washed with methylene chloride and acetonitrile to afford the title compound after drying in vacuo (268 mg, 70%); HRMS (ESI−): calculated for C₃₆H₂₈N₂O₁₂S m/z [M−H]⁻: 842.8801, observed 842.8799; ¹H NMR δ 10.84 (s, 2H), 8.84 (s, 2H), 8.17 (d, J=6.9 Hz, 2H), 7.73 (s, 2H), 7.49-7.63 (m, 6H), 7.36-7.49 (d, J=3.9 Hz, 2H), 7.20-7.36 (m, 2H)

### Example 106—Synthesis of N-cyclopropyl-5-{[2-(cyclopropylcarbamoyl)-1,3-dioxo-2,3-dihydro-1H-inden-5-yl]oxy}-1,3-dioxo-2,3-dihydro-1H-indene-2-carboxamide

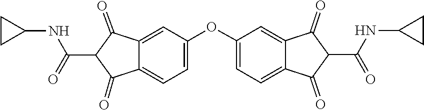 [SMILES: O=C(NC1CC1)C1C(=O)c2ccc(Oc3ccc4c(c3)C(=O)C(C(=O)NC3CC3)C4=O)cc2C1=O]

A solution of 5-[(1,3-dioxo-2,3-dihydro-1H-inden-5-yl)oxy]-2,3-dihydro-1H-indene-1,3-dione (Example 7, 0.63 mmol), in anhydrous DMF (5.0 ml) was cooled to −70° C. Triethylamine (439 μL, 3.15 mmol) was added slowly over 5 min, followed by dropwise addition of cyclopropyl isocyanate (CAS number 4747-72-2, 2.52 mmol, 4.0 eq.), and the resulting mixture was stirred at −70° C. for 10 min then warmed to room temperature, monitoring by LCMS. The product was isolated by treatment with 1N HCl and filtered to provide the title compound as a dark brown solid (201 mg, 68%); HRMS (ESI−): calculated for C₂₆H₁₉N₂O₇ m/z [M−H]⁻: 471.1198, observed 471.1207; ¹H NMR δ 8.65 (bs, 2H), 7.61 (d, J=7.6 Hz, 2H), 7.28 (d, J=7.6 Hz, 2H), 7.18 (s, 2H), 2.9-2.82 (m, 2H), 0.83-0.72 (m, 8H).

### Example 107—Synthesis of N-cyclopentyl-5-{[2-(cyclopentylcarbamoyl)-1,3-dioxo-2,3-dihydro-1H-inden-5-yl]oxy}-1,3-dioxo-2,3-dihydro-1H-indene-2-carboxamide

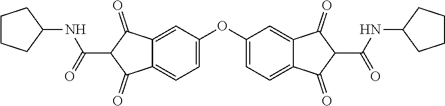 [SMILES: O=C(NC1CCCC1)C1C(=O)c2ccc(Oc3ccc4c(c3)C(=O)C(C(=O)NC3CCCC3)C4=O)cc2C1=O]

The title compound was prepared according to the procedure for Example 106 using 5-[(1,3-dioxo-2,3-dihydro-1H-inden-5-yl)oxy]-2,3-dihydro-1H-indene-1,3-dione (Example 7, 0.63 mmol) and cyclopentyl isocyanate (CAS number 4747-71-1, 2.52 mmol). The product was isolated by treatment with 1N HCl, filtrated then dissolved in 1N NaOH. The basic aqueous was washed with dichloromethane then re-acidified with 1N HCl and filtered. A final trituration with methanol afforded a dark brown solid (84 mg, 25%); HRMS (ESI−): calculated for C₃₀H₂₇N₂O₇ m/z [M−H]⁻: 527.1824, observed 527.1818; ¹H NMR δ 8.62-8.50 (m, 2H), 7.60 (d, J=7.6 Hz, 2H), 7.28-7.14 (m, 4H), 4.28-4.15 (m, 2H), 1.98-1.87 (m, 4H), 1.72-1.45 (m, 12H).

### Example 108—Synthesis of N-cyclopropyl-5-{2-[2-(cyclopropylcarbamoyl)-1,3-dioxo-2,3-dihydro-1H-inden-5-yl]-1,1,1,3,3,3-hexafluoropropan-2-yl}-1,3-dioxo-2,3-dihydro-1H-indene-2-carboxamide

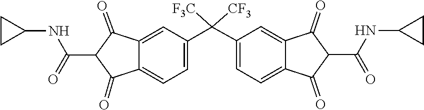 [SMILES: O=C(NC1CC1)C1C(=O)c2ccc(C(c3ccc4c(c3)C(=O)C(C(=O)NC3CC3)C4=O)(C(F)(F)F)C(F)(F)F)cc2C1=O]

A solution of 5-(2,2,2-trifluoro-1-(5-indan-1,3-dionyl)-1-trifluoromethyl-1-ethyl)-indan-1,3-dione (Example 13, 277 mg, 0.63 mmol) was cooled to −70° C. Triethylamine (439 μL, 3.15 mmol) was added slowly over 5 min, followed by dropwise addition of cyclopropyl isocyanate (CAS number 4747-72-2, 2.52 mmol), and the resulting mixture was stirred at −70° C. for 10 min then warmed to room temperature, monitoring by LCMS. The product was isolated by treatment with 1N HCl and filtered to provide the title compound as a dark brown solid (311 mg, 81%); HRMS (ESI−): calculated for C₂₉H₁₉F₆N₂O₆ m/z [M−H]−: 605.1153, observed 605.1152; ¹H NMR δ 8.88-8.73 (m, 2H), 7.70-7.60 (m, 4H), 7.42-7.33 (m, 2H), 2.90-2.85 (m, 2H), 0.82-0.71 (m, 8H).

### Example 109—Synthesis of N-cyclopentyl-5-{2-[2-(cyclopentylcarbamoyl)-1,3-dioxo-2,3-dihydro-1H-inden-5-yl]-1,1,1,3,3,3-hexafluoropropan-2-yl}-1,3-dioxo-2,3-dihydro-1H-indene-2-carboxamide

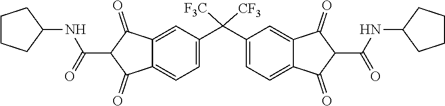 [SMILES: O=C(NC1CCCC1)C1C(=O)c2ccc(C(c3ccc4c(c3)C(=O)C(C(=O)NC3CCCC3)C4=O)(C(F)(F)F)C(F)(F)F)cc2C1=O]

The title compound was prepared according to the procedure for Example 108 using 5-(2,2,2-trifluoro-1-(5-indan-1,3-dionyl)-1-trifluoromethyl-1-ethyl)-indan-1,3-dione (Example 13, 277 mg, 0.63 mmol) and cyclopentyl isocyanate (CAS number 4747-71-1, 260 μL, 2.52 mmol). The product was isolated by treatment with 1N HCl, filtration then dissolution in 1N NaOH. The basic aqueous was washed with dichloromethane and chloroform then re-acidified with 1 N HCl and filtered. A final trituration with methanol afforded a dark brown solid (78 mg, 12%); HRMS (ESI−): calculated for C₃₃H₂₇F₆N₂O₆ m/z [M−H]⁻: 661.1779, observed 661.1758; ¹H NMR δ 8.89-8.65 (m, 2H), 7.74-7.53 (m, 4H), 7.46-7.34 (m, 2H), 4.28-4.15 (m, 2H), 1.98-1.86 (m, 4H), 1.75-1.49 (m, 12H).

### Example 110—Synthesis of (2Z,2′Z)-2,2′-bis[(cyclopropylamino)(hydroxyl) methylidene]-1H,1′H,2H,2′H,3H,3′H-[5,5′-biindene]-1,1′,3,3′-tetrone

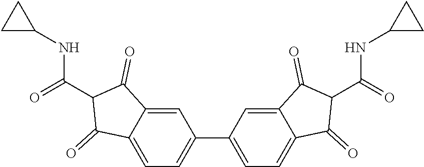 [SMILES: O=C(NC1CC1)C1C(=O)c2ccc(-c3ccc4c(c3)C(=O)C(C(=O)NC3CC3)C4=O)cc2C1=O]

A solution of 1H,1′H,2H,2′H,3H,3′H-[5,5′-biindene]-1,1′,3,3′-tetrone (100 mg, 0.34 mmol) and DMF 5.0 ml was cooled in a dry ice acetone bath. Triethylamine (240.3 μl, 1.72 mmol) was added and after 5 min. Cyclopropyl isocyanate (CAS number 4747-72-2, 108.7 μl, 1.37 mmol) was added dropwise. The contents were then stirred for 18 hours at room temperature. Once LCMS confirmed the reaction to be complete the reaction vessel was placed on a cracked ice water bath. Then, 2N HCl 10.0 ml was added the resulting solid was collected and washed with 25 ml of 2N HCl and 25 ml of Et₂O. The collected solid was air dried overnight then added to an excess of 1N NaOH 10 ml and 10 ml of CH₂Cl₂ and sonicated for 10 min. The resulting suspension was filtered and dried to provide the title compound as a dark colored bis sodium salt (121 mg, 73% yield). LCMS (ESI+): R_{f}=5.67; HRMS (ESI−): calculated for C₂₆H₁₉N₂O₆ m/z [M−H]⁻: 455.124992, observed 455.124860; ¹H NMR δ: 8.50-8.62 (m, 2H), 7.67-7.74 (s, 2H), 7.47-7.53 (m, 2H), 7.36 (d, J=7.6 Hz, 2H), 2.62-2.78 (m, 2H), 0.64 (br d, J=6.7 Hz, 4H), 0.27-0.51 (m, 4H)

### Example 111—Synthesis of tert-butyl (2S)-2-(5-{2-[(2S)-1-[(tert-butoxy)carbonyl]pyrrolidine-2-carbonyl]-1,3-dioxo-2,3-dihydro-1H-indene-5-carbonyl}-1,3-dioxo-2,3-dihydro-1H-indene-2-carbonyl)pyrrolidine-1-carboxylate

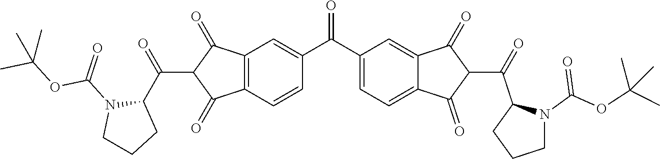 [SMILES: CC(C)(C)OC(=O)N1CCC[C@H]1C(=O)C1C(=O)c2ccc(C(=O)c3ccc4c(c3)C(=O)C(C(=O)[C@@H]3CCCN3C(=O)OC(C)(C)C)C4=O)cc2C1=O]

A mixture of DMAP (183 mg, 1.50 mmol), N-BOC-L-proline (CAS number 15761-39-4, 322 mg, 1.50 mmol) and 9.0 ml of anhydrous DMF was stirred for 10 min after which a solution had formed. EDCI HCl (CAS number 25952-53-8, 286.5 mg, 1.50 mmol) was added and the contents were stirred for additional 10 min. 5,5′-carbonylbis(1H-indene-1,3(2H)-dione) (Example 4, 160.0 mg, 0.5 mmol) was added and the reaction mixture was stirred overnight. The following day 15.0 ml of 2 N HCl was added and the mixture was stirred for 5 min. The resulting precipitant was filtered from solution. The solid was washed once with 5 ml of 2N HCl and 5 ml of water. The dried solid was milled into a fine powder and was treated with 10 ml of 1N NaOH and 5 ml of methylene chloride. This biphasic suspension was shaken vigorously and filtered leaving a fine black powder. The titled compound (251.2 mg, 66%) was obtained as a dark solid bis-sodium salt. LCMS (ESI+): Rf=5.67, HRMS (ESI−): calculated for C₃₉H₃₉N₂O₁₁ m/z [M−H]⁻: 711.255934, observed 711.253953; ¹H NMR δ: 7.86 (dd, J=7.4 Hz, 2H), 7.60-7.69 (s, 2H), 7.48-7.59 (dd, 2H), 5.24-5.48 (m, 2H), 3.32 (m, 4H), 2.12 (m, 4H), 1.53-1.81 (m, 4H), 1.27-1.42 (s, 9H), 1.11-1.25 (s, 9H).

### Example 112—Synthesis of tert-butyl (2S)-2-{2-[5-(2-{2-[(2S)-1-[(tert-butoxy)carbonyl]pyrrolidin-2-yl]acetyl}-1,3-dioxo-2,3-dihydro-1H-indene-5-carbonyl)-1,3-dioxo-2,3-dihydro-1H-inden-2-yl]-2-oxoethyl}pyrrolidine-1-carboxylate

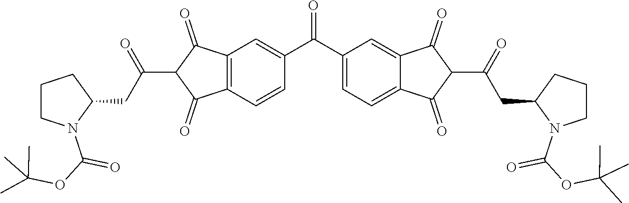 [SMILES: CC(C)(C)OC(=O)N1CCC[C@@H]1CC(=O)C1C(=O)c2ccc(C(=O)c3ccc4c(c3)C(=O)C(C(=O)C[C@H]3CCCN3C(=O)OC(C)(C)C)C4=O)cc2C1=O]

In a similar manner to that of Example 111, the title compound was prepared from 5,5′-carbonylbis(1H-indene-1,3(2H)-dione) (Example 4, 160.0 mg, 0.5 mmol) and BOC-L-beta-homoproline (CAS number 56502-01-3, 343.1 mg, 1.50 mmol) to provide a dark solid bis-sodium salt (128 mg, 32% yield). LCMS (ESI+): R_{f}=6.97, HRMS (ESI−): calculated for C₄₁H₄₃N₂O₁₁ m/z [M−H]⁻: 739.287234, observed 739.285411; ¹H NMR δ: 7.85 (dd, J=1.0 Hz, 2H), 7.62 (s, 2H), 7.53 (d, J=7.4 Hz, 2H), 4.13 (m, 2H), 3.14-3.29 (m, 8H), 2.92 (m, 2H), 1.85 (m, 4H), 1.71 (m, 2H), 1.36 (br s, 18H).

### Example 113—Synthesis of tert-butyl (2S,4R)-2-(5-{2-[(2S,4R)-1-[(tert-butoxy)carbonyl]-4-fluoropyrrolidine-2-carbonyl]-1,3-dioxo-2,3-dihydro-1H-indene-5-carbonyl}-1,3-dioxo-2,3-dihydro-1H-indene-2-carbonyl)-4-fluoropyrrolidine-1-carboxylate

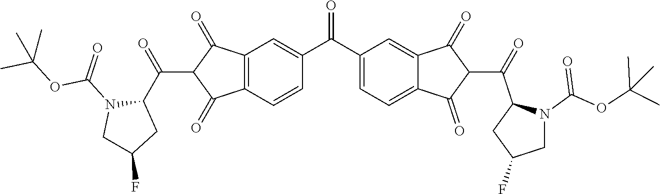 [SMILES: CC(C)(C)OC(=O)N1C[C@H](F)C[C@H]1C(=O)C1C(=O)c2ccc(C(=O)c3ccc4c(c3)C(=O)C(C(=O)[C@@H]3C[C@@H](F)CN3C(=O)OC(C)(C)C)C4=O)cc2C1=O]

In a similar manner to that of Example 111, the title compound was prepared from 5,5′-carbonylbis(1H-indene-1,3(2H)-dione) (Example 4, 160.0 mg, 0.5 mmol) and (2S, 4R)-1-[(tert-butoxy)carbonyl]-4-fluoropyrrolidine-2-carboxylic acid (CAS number: 203866-14-2, 349.55 mg, 1.50 mmol) to provide a dark solid bis-sodium salt (261 mg, 66% yield). HRMS (ESI−): calculated for C₃₉H₃₇F₂N₂O₁₁ m/z [M−H]⁻: 747.2371, observed 747.2359; ¹H NMR δ: 7.87 (dd, J=1.0 Hz, 2H), 7.65 (s, 2H), 7.56 (dd, J=7.6, 2.0 Hz, 2H), 5.43-5.67 (m, 2H), 5.07-5.41 (m, 2H), 3.56-3.82 (m, 2H), 3.42-3.55 (m, 2H), 2.49-2.63 (m, 2H), 1.59-1.98 (m, 2H), 1.36 (s, 6H), 1.15-1.25 (s, 12H).

### Example 114—Synthesis of methyl (2S)-2-(5-{2-[(2S)-1-(methoxycarbonyl)pyrrolidine-2-carbonyl]-1,3-dioxo-2,3-dihydro-1H-indene-5-carbonyl}-1,3-dioxo-2,3-dihydro-1H-indene-2-carbonyl)pyrrolidine-1-carboxylate

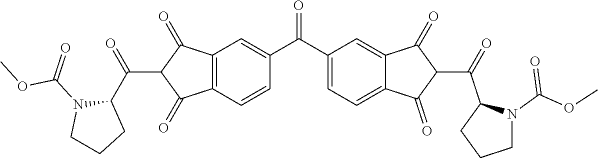

In a similar manner to that of Example 111, the title compound was prepared from 5,5′-carbonylbis(1H-indene-1,3(2H)-dione) (Example 4, 160.0 mg, 0.5 mmol) and (2S)-1-(methoxycarbonyl)pyrrolidine-2-carboxylic acid (CAS number 74761-41-4, 256.6 mg, 1.50 mmol) to provide a dark solid bis-sodium salt (228 mg, 68% yield). LCMS (ESI+): Rf=4.40, (M−H)+, HRMS (ESI−): calculated for C₃₃H₂₇N₂O₁₁ m/z [M−H]⁻: 627.162033, observed 627.163608; ¹H NMR δ: 7.88 (dd, J=7.6, 1.5 Hz, 2H), 7.65 (s, 2H), 7.56 (d, J=7.6 Hz, 2H), 5.32-5.40 (m, 2H), 3.52 (s, 3H), 3.42 (s, 3H), 3.27-3.39 (m, 4H), 2.06-2.34 (m, 2H), 1.58-1.84 (m, 6H).

### Example 115—Synthesis of tert-butyl (2S)-2-(5-{2-[(2S)-1-[(tert-butoxy)carbonyl]azetidine-2-carbonyl]-1,3-dioxo-2,3-dihydro-1H-indene-5-carbonyl}-1,3-dioxo-2,3-dihydro-1H-indene-2-carbonyl)azetidine-1-carboxylate

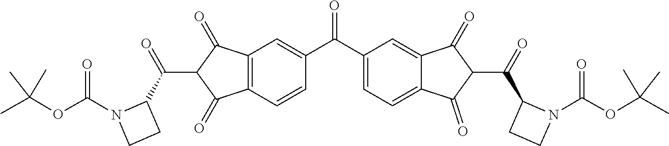

In a similar manner to that of Example 111, the title compound was prepared from 5,5′-carbonylbis(1H-indene-1,3(2H)-dione) (Example 4, 160.0 mg, 0.5 mmol) and BOC-L-azetidine-2-carboxylic acid (CAS number 51077-14-6, 301.5 mg, 1.50 mmol) to provide a dark solid bis-sodium salt (212 mg, 58% yield). LCMS (ESI+): R_{f}=5.73, (M−H)+; HRMS (ESI−): calculated for C₃₇H₃₅N₂O₁₁ m/z [M−H]⁻: 683.224634, observed 683.226126; ¹H NMR δ: 8.10 (dd, J=7.6, 1.7 Hz, 2H), 7.76 (s, 2H), 7.57 (d, J=7.7 Hz, 2H), 5.36-5.61 (m, 2H), 3.55-3.82 (m, 4H), 1.54-1.82 (m, 2H), 1.34 (s, 8H), 1.22 (s, 10H).

### Example 116—Synthesis of 2-[(2S)-azetidine-2-carbonyl]-5-{2-[(2S)-azetidine-2-carbonyl]-1,3-dioxo-2,3-dihydro-1H-indene-5-carbonyl}-2,3-dihydro-1H-indene-1,3-dione

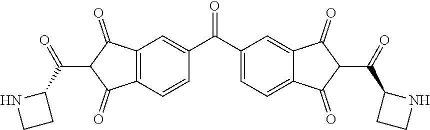 [SMILES: O=C(c1ccc2c(c1)C(=O)C(C(=O)[C@@H]1CCN1)C2=O)c1ccc2c(c1)C(=O)C(C(=O)[C@@H]1CCN1)C2=O]

To a stirred mixture of tert-butyl (2S)-2-(5-{2-[(2S)-1-[(tert-butoxy)carbonyl]azetidine-2-carbonyl]-1,3-dioxo-2,3-dihydro-1H-indene-5-carbonyl}-1,3-dioxo-2,3-dihydro-1H-indene-2-carbonyl)azetidine-1-carboxylate (Example 115, 100.0 mg, 0.137 mmol) and MeOH (10 ml) in an ice bath was added trifluoroacetic acid (1.0 ml, 5.9 mmol) dropwise and the solution stirred overnight. The reaction mixture was concentrated and the resulting solid was stirred briefly in 5.0 ml 1N NaOH and filtered to provide the title compound as a dark solid bis-sodium salt (56 mg, 78% yield). LCMS (ESI+): R_{f}=2.22, (M−H)+; HRMS (ESI−): calculated for C₂₇H₁₉N₂O₇ m/z [M−H]⁻: 483.1198, observed 483.1175; ¹H NMR δ: 8.10 (dd, J=7.6, 1.7 Hz, 2H), 7.76 (s, 2H), 7.57 (d, J=7.7 Hz, 2H), 5.36-5.61 (m, 2H), 3.55-3.82 (m, 4H), 1.73-1.88 (m, 2H), 1.54-1.82 (m, 2H).

### Example 117—Synthesis of 5-{1,3-dioxo-2-[(2S)-pyrrolidine-2-carbonyl]-2,3-dihydro-1H-indene-5-carbonyl}-2-[(2S)-pyrrolidine-2-carbonyl]-2,3-dihydro-1H-indene-1,3-dione

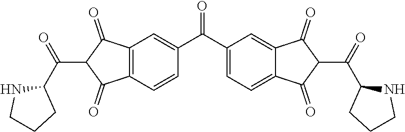 [SMILES: O=C(c1ccc2c(c1)C(=O)C(C(=O)[C@@H]1CCCN1)C2=O)c1ccc2c(c1)C(=O)C(C(=O)[C@@H]1CCCN1)C2=O]

In a similar manner to that of Example 116, tert-butyl (2S)-2-(5-{2-[(2S)-1-[(tert-butoxy)carbonyl]pyrrolidine-2-carbonyl]-1,3-dioxo-2,3-dihydro-1H-indene-5-carbonyl}-1,3-dioxo-2,3-dihydro-1H-indene-2-carbonyl)pyrrolidine-1-carboxylate (Example 111, 100.0 mg, 0.14 mmol) was used to prepare the title compound (55.2 mg, 70% yield). LCMS (ESI+): R_{f}=2.50, (M−H)+; HRMS (ESI−): calculated for C₂₉H₂₃N₂O₇ m/z [M−H]⁻: 511.151075, observed 511.150983; ¹H NMR (methanol-d₄) δ: 7.97 (dd, J=1.0 Hz, 2H), 7.88 (s, 2H), 7.66 (d, J=1.0 Hz, 2H), 4.68 (m, 2H), 3.05-3.20 (m, 4H), 2.71-2.86 (m, 2H), 2.30-2.45 (m, 2H), 1.73-1.88 (m, 2H), 1.50-1.71 (m, 4H).

### Example 118—Synthesis of 2-[(2S)-2-amino-2-cyclopropylacetyl]-5-{2-[(2S)-2-amino-2-cyclopropylacetyl]-1,3-dioxo-2,3-dihydro-1H-indene-5-carbonyl}-2,3-dihydro-1H-indene-1,3-dione

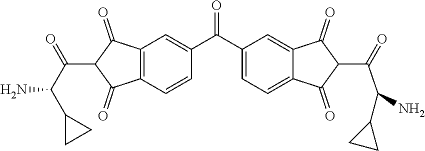 [SMILES: N[C@H](C(=O)C1C(=O)c2ccc(C(=O)c3ccc4c(c3)C(=O)C(C(=O)[C@@H](N)C3CC3)C4=O)cc2C1=O)C1CC1]

In a similar manner to that of Example 111, the title compound was prepared from 5,5′-carbonylbis(1H-indene-1,3(2H)-dione) (Example 4, 160.0 mg, 0.5 mmol) and BOC-L-cyclopropylglycine (CAS Number: 155976-13-9, 322.6 mg, 1.50 mmol). During the course of the workup procedure the Boc group was inadvertently removed to provide the title compound as a dark solid bis sodium salt (128.2 mg, 32%). LCMS (ESI+): R_{f}=6.97; HRMS (ESI−): calculated for C₂₈H₁₇N₄O₈S m/z [M−H]⁻: 511.151075, observed 511.150983; ¹H NMR δ: 7.86 (dd, J=7.5, 1.4 Hz, 2H), 7.63 (s, 2H), 7.54 (d, J=7.5 Hz, 2H), 4.23 (q, J=7.1 Hz, 2H), 1.30-1.46 (m, 4H), 0.87-1.02 (m, 2H), 0.30-0.48 (m, 2H), 0.00-0.28 (m, 6H).

### Example 119—Synthesis of 2-[(2R)-2-methoxy-2-phenylacetyl]-5-{2-[(2R)-2-methoxy-2-phenylacetyl]-1,3-dioxo-2,3-dihydro-1H-indene-5-carbonyl}-2,3-dihydro-1H-indene-1,3-dione

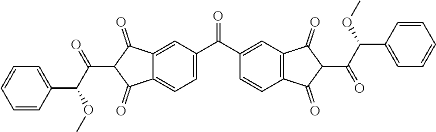 [SMILES: CO[C@@H](C(=O)C1C(=O)c2ccc(C(=O)c3ccc4c(c3)C(=O)C(C(=O)[C@H](OC)c3ccccc3)C4=O)cc2C1=O)c1ccccc1]

In a similar manner to that of Example 111, the title compound was prepared from 5,5′-carbonylbis(1H-indene-1,3(2H)-dione) (Example 4, 160.0 mg, 0.5 mmol) and (2R)-methoxy(phenyl)acetic acid (CAS number 3966-32-3, 249.0 mg, 1.50 mmol) to provide a dark solid bis-sodium salt (274 mg, 84% yield). LCMS (ESI+): R_{f}=6.87, (M−H)+; HRMS (ESI−): calculated for C₃₇H₂₅O₉ m/z [M−H]⁻: 613.1504, observed 613.1501; ¹H NMR δ: 7.88 (dd, J=7.6, 1.5 Hz, 2H), 7.65 (s, 2H), 7.56 (d, J=7.6 Hz, 2H), 5.32-5.40 (m, 2H), 3.52 (s, 3H), 3.42 (s, 3H), 3.27-3.39 (m, 4H), 2.06-2.34 (m, 2H), 1.58-1.84 (m, 6H).

### Example 120A—Synthesis of 5-{2-[1-hydroxy-2-(4-methylphenyl)propyl]-1,3-dioxo-2,3-dihydro-1H-indene-5-carbonyl}-2-[2-(4-methylphenyl)propanoyl]-2,3-dihydro-1H-indene-1,3-dione

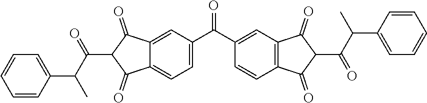 [SMILES: CC(C(=O)C1C(=O)c2ccc(C(=O)c3ccc4c(c3)C(=O)C(C(=O)C(C)c3ccccc3)C4=O)cc2C1=O)c1ccccc1]

In a similar manner to that of Example 111, the title compound was prepared from 5,5′-carbonylbis(1H-indene-1,3(2H)-dione) (Example 4, 160.0 mg, 0.5 mmol) and 2-(4-methylphenyl)propanoic acid (CAS number 938-94-3, 246 mg, 1.50 mmol) to provide a dark solid bis-sodium salt (228 mg, 74% yield). LCMS (ESI+): R_{f}=7.32, (M−H)+; HRMS (ESI−): calculated for C₃₉H₂₉O₇ m/z [M−H]⁻: 609.191877, observed 609.189708; ¹H NMR δ: 7.82 (dd, J=7.5, 1.5 Hz, 2H), 7.59 (s, 2H), 7.51 (d, J=7.5 Hz, 2H), 7.19 (d, J=8.1 Hz, 4H), 6.99 (d, J=7.8 Hz, 4H), 5.23 (q, J=7.1 Hz, 2H), 2.19 (s, 6H), 1.24 (d, J=7.0 Hz, 6H).

### Example 120B—Synthesis of tert-butyl (2S,5R)-2-(5-{2-[(2S,5R)-1-[(tert-butoxy)carbonyl]-5-methylpyrrolidine-2-carbonyl]-1,3-dioxo-2,3-dihydro-1H-indene-5-carbonyl}-1,3-dioxo-2,3-dihydro-1H-indene-2-carbonyl)-5-methylpyrrolidine-1-carboxylate

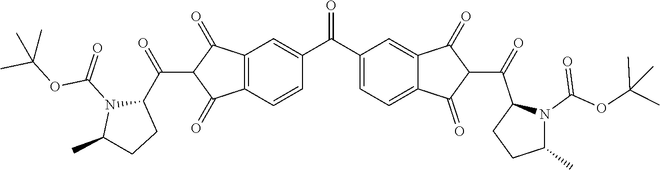 [SMILES: C[C@@H]1CC[C@@H](C(=O)C2C(=O)c3ccc(C(=O)c4ccc5c(c4)C(=O)C(C(=O)[C@@H]4CC[C@@H](C)N4C(=O)OC(C)(C)C)C5=O)cc3C2=O)N1C(=O)OC(C)(C)C]

In a similar manner to that of Example 111, the title compound was prepared from 5,5′-carbonylbis(1H-indene-1,3(2H)-dione) (Example 4, 160.0 mg, 0.5 mmol) and (2S,5R)-1-(tert-butoxycarbonyl)-5-methylpyrrolidine-2-carboxylic acid (CAS number 160033-52-3, 322.5 mg, 1.50 mmol) to provide a dark solid bis-sodium salt (205 mg, 52% yield). LCMS (ESI+): R_{f}=7.00, (M−H)+; HRMS (ESI−): calculated for C₄₁H₄₃N₂O₁₁ m/z [M−H]⁻: 739.2872, observed 739.2865; ¹H NMR δ: 7.86 (br. d, J=7.7 Hz, 2H), 7.64 (s, 2H), 7.54 (br d, J=7.7 Hz, 2H), 5.45 (s, 2H), 3.81 (m, 2H), 2.06-2.16 (m, 2H), 1.85-1.95 (m, 2H), 1.61 (m, 2H), 1.43 m, 2H), 1.36 (s, 8H), 1.16-1.29 (m, 16H),

### Example 121—Synthesis of 2-[(2S,3R)-2-amino-3-methoxybutanoyl]-5-{2-[(2S,3R)-2-amino-3-methoxybutanoyl]-1,3-dioxo-2,3-dihydro-1H-indene-5-carbonyl}-2,3-dihydro-1H-indene-1,3-dione

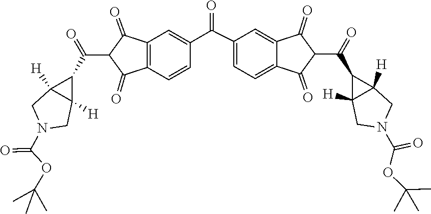 [SMILES: CC(C)(C)OC(=O)N1C[C@@H]2[C@H](C1)[C@H]2C(=O)C1C(=O)c2ccc(C(=O)c3ccc4c(c3)C(=O)C(C(=O)[C@H]3[C@@H]5CN(C(=O)OC(C)(C)C)C[C@@H]53)C4=O)cc2C1=O]

In a similar manner to that of Example 111, the title compound was prepared from 5,5′-carbonylbis(1H-indene-1,3(2H)-dione) (Example 4,160.0 mg, 0.5 mmol) and (1R,5S,6R)-3-(tert-butoxycarbonyl)-3-azabicyclo[3.1.0]hexane-6-carboxylic acid (CAS number 927679-54-7, 340.5 mg, 1.50 mmol) to provide a dark solid bis-sodium salt (281 mg, 72% yield). LCMS (ESI+): R_{f}=6.33, (M−H)+; HRMS (ESI−): calculated for C₄₁H₃₉N₂O₁₁ m/z [M−H]⁻: 735.255934, observed 735.256939; ¹H NMR δ: 7.88 (dd, J=1.0 Hz, 2H), 7.65 (s, 2H), 7.54 (d, J=7.5 Hz, 2H), 3.42-3.53 (m, 4H), 3.34-3.41 (m, 2H), 3.17-3.29 (m, 4H), 1.90 (s, 4H), 1.40 (s, 18H).

### Example 122—Synthesis of tert-butyl (2S)-2-(5-{2-[(2S)-1-[(tert-butoxy)carbonyl]-2,5-dihydro-1H-pyrrole-2-carbonyl]-1,3-dioxo-2,3-dihydro-1H-indene-5-carbonyl}-1,3-dioxo-2,3-dihydro-1H-indene-2-carbonyl)-2,5-dihydro-1H-pyrrole-1-carboxylate

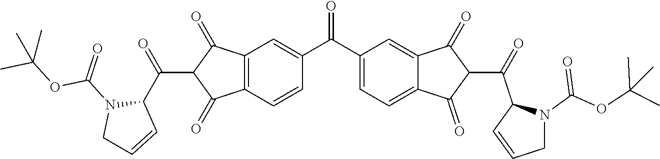 [SMILES: CC(C)(C)OC(=O)N1CC=C[C@H]1C(=O)C1C(=O)c2ccc(C(=O)c3ccc4c(c3)C(=O)C(C(=O)[C@@H]3C=CCN3C(=O)OC(C)(C)C)C4=O)cc2C1=O]

In a similar manner to that of Example 111, the title compound was prepared from 5,5′-carbonylbis(1H-indene-1,3(2H)-dione) (Example 4, 160.0 mg, 0.5 mmol) and (2S)-1-(tert-butoxycarbonyl)-2,5-dihydro-1H-pyrrole-2-carboxylic acid (CAS number 800412-56-0, 409.5 mg, 1.50 mmol) to provide a dark solid bis-sodium salt (279 mg, 75% yield). LCMS (ESI+): R_{f}=6.06, (M−H)+; HRMS (ESI−): calculated for C₃₉H₃₅N₂O₁₁ m/z [M−H]⁻: 707.224634, observed 707.226201; ¹H NMR δ: 7.83-7.92 (m, 2H), 7.66 (s, 2H), 7.51-7.61 (m, 2H), 5.88-6.19 (m, 2H), 5.57-5.94 (m, 4H), 4.04 (s, 4H), 1.38 (s, 8H), 1.23 (s, 10H).

### Example 123—Synthesis of tert-butyl (3R)-3-(5-{2-[(3R)-1-[(tert-butoxy)carbonyl]pyrrolidine-3-carbonyl]-1,3-dioxo-2,3-dihydro-1H-indene-5-carbonyl}-1,3-dioxo-2,3-dihydro-1H-indene-2-carbonyl)pyrrolidine-1-carboxylate

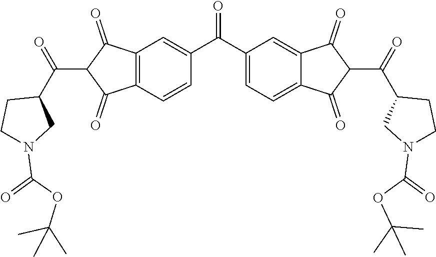 [SMILES: CC(C)(C)OC(=O)N1CC[C@@H](C(=O)C2C(=O)c3ccc(C(=O)c4ccc5c(c4)C(=O)C(C(=O)[C@@H]4CCN(C(=O)OC(C)(C)C)C4)C5=O)cc3C2=O)C1]

In a similar manner to that of Example 111, the title compound was prepared from 5,5′-carbonylbis(1H-indene-1,3(2H)-dione) (Example 4, 160.0 mg, 0.5 mmol) and (3R)-1,3-pyrrolidinedicarboxylic acid, 1-(1,1-dimethylethyl) ester (CAS number 72925-16-7, 343.1 mg, 1.50 mmol) to provide a dark solid bis-sodium salt (128 mg, 32% yield). LCMS (ESI+): R_{f}=6.64, (M−H)+; HRMS (ESI−): calculated for C₃₉H₃₉N₂O₁₁ m/z [M−H]⁻: 711.255934, observed 711.252970; ¹H NMR δ: 7.85 (dd, J=1.0 Hz, 2H), 7.62 (s, 2H), 7.53 (d, J=7.4 Hz, 2H), 4.13 (m, 2H), 3.14-3.29 (m, 8H), 2.92 (m, 2H), 1.85 (m, 4H), 1.71 (m, 2H), 1.36 (br s, 18H).

### Example 124—Synthesis of tert-butyl 2-[5-(2-{3-[(tert-butoxy)carbonyl]-1,3-thiazolidine-2-carbonyl}-1,3-dioxo-2,3-dihydro-1H-indene-5-carbonyl)-1,3-dioxo-2,3-dihydro-1H-indene-2-carbonyl]-1,3-thiazolidine-3-carboxylate

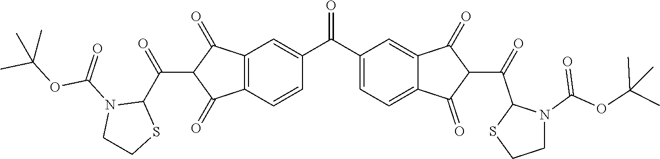 [SMILES: CC(C)(C)OC(=O)N1CCSC1C(=O)C1C(=O)c2ccc(C(=O)c3ccc4c(c3)C(=O)C(C(=O)C3SCCN3C(=O)OC(C)(C)C)C4=O)cc2C1=O]

In a similar manner to that of Example 111, the title compound was prepared from 5,5′-carbonylbis(1H-indene-1,3(2H)-dione) (Example 4, 160.0 mg, 0.5 mmol) and 3-(tert-butoxycarbonyl)-1,3-thiazolidine-2-carboxylic acid (CAS number 141783-63-3, 349.6 mg, 1.50 mmol) to provide a dark solid bis-sodium salt (217.2 mg, 55% yield). LCMS (ESI+): R_{f}=6.38, (M−H)+, HRMS (ESI−): calculated for C₃₇H₃₅N₂O₁₁S₂ m/z [M−H]⁻: 747.168776, observed 747.167609; ¹H NMR δ: 7.84-7.92 (m, 2H), 7.67 (s, 2H), 7.50-7.62 (m, 2H), 6.29-6.36 (m, 2H), 3.63-3.79 (m, 4H), 2.85-3.06 (m, 4H), 1.40 (s, 8H), 1.26 (s, 10H).

### Example 125—Synthesis of benzyl (2S)-2-(5-{2-[(2S)-1-[(benzyloxy)carbonyl]pyrrolidine-2-carbonyl]-1,3-dioxo-2,3-dihydro-1H-indene-5-carbonyl}-1,3-dioxo-2,3-dihydro-1H-indene-2-carbonyl)pyrrolidine-1-carboxylate

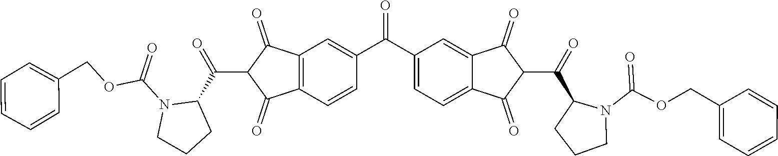 [SMILES: O=C(c1ccc2c(c1)C(=O)C(C(=O)[C@@H]1CCCN1C(=O)OCc1ccccc1)C2=O)c1ccc2c(c1)C(=O)C(C(=O)[C@@H]1CCCN1C(=O)OCc1ccccc1)C2=O]

In a similar manner to that of Example 111, the title compound was prepared from 5,5′-carbonylbis(1H-indene-1,3(2H)-dione) (Example 4, 160.0 mg, 0.5 mmol) and CBZ-L-proline (CAS Number 1148-11-4, 373.5 mg, 1.50 mmol) to provide a dark solid bis-sodium salt (284 mg, 69% yield). LCMS (ESI+): R_{f}=6.67, (M−H)+; HRMS (ESI−): calculated for C₄₅H₃₅N₂O₁₁ m/z [M−H]⁻: 779.224634, observed 779.224200; ¹H NMR δ: 7.83-7.93 (m, 2H), 7.61-7.71 (m, 2H), 7.56 (d, J=7.7 Hz, 2H), 7.29-7.41 (m, 5H), 7.07-7.26 (m, 5H), 5.37-5.61 (m, 2H), 4.86-5.11 (m, 4H), 3.36-3.56 (m, 4H), 1.99-2.32 (m, 2H), 1.61-1.87 (m, 6H).

### Example 126—Synthesis of 5-{1,3-dioxo-2-[2-(1H-pyrazol-1-yl)acetyl]-2,3-dihydro-1H-indene-5-carbonyl}-2-[2-(1H-pyrazol-1-yl)acetyl]-2,3-dihydro-1H-indene-1,3-dione

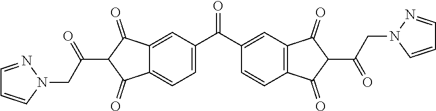 [SMILES: O=C(c1ccc2c(c1)C(=O)C(C(=O)Cn1cccn1)C2=O)c1ccc2c(c1)C(=O)C(C(=O)Cn1cccn1)C2=O]

In a similar manner to that of Example 111, the title compound was prepared from 5,5′-carbonylbis(1H-indene-1,3(2H)-dione) (Example 4, 160.0 mg, 0.5 mmol) and 2-(1H-pyrazol-1-yl)acetic acid (CAS number 16034-48-3, 189.0 mg, 1.50 mmol) to provide a dark solid bis-sodium salt (135.0 mg, 47% yield). LCMS (ESI+): Rf=6.64, (M−H)+; HRMS (ESI−): calculated for C₂₉H₁₇N₄O₇ m/z [M−H]⁻: 533.1093, observed 533.1103; ¹H NMR δ: 7.90 (dd, J=7.5, 1.6 Hz, 2H), 7.69 (s, 2H), 7.57-7.64 (m, 4H), 7.36 (d, J=1.8 Hz, 2H), 6.20 (s, 2H), 5.39 (s, 4H).

### Example 127—Synthesis of tert-butyl (2S)-2-(5-{2-[(2S)-1-[(tert-butoxy)carbonyl]piperidine-2-carbonyl]-1,3-dioxo-2,3-dihydro-1H-indene-5-carbonyl}-1,3-dioxo-2,3-dihydro-1H-indene-2-carbonyl)piperidine-1-carboxylate

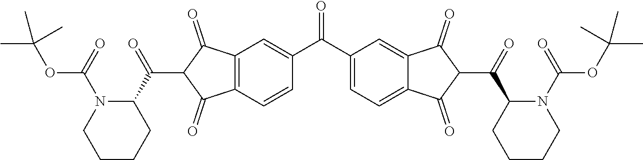 [SMILES: CC(C)(C)OC(=O)N1CCCC[C@H]1C(=O)C1C(=O)c2ccc(C(=O)c3ccc4c(c3)C(=O)C(C(=O)[C@@H]3CCCCN3C(=O)OC(C)(C)C)C4=O)cc2C1=O]

In a similar manner to that of Example 111, the title compound was prepared from 5,5′-carbonylbis(1H-indene-1,3(2H)-dione) (Example 4, 160.0 mg, 0.5 mmol) and (2S)-1-(tert-butoxycarbonyl)piperidine-2-carboxylic acid (CAS number 26250-84-0, 343.6 mg, 1.50 mmol) to provide a dark solid bis-sodium salt (302 mg 77% yield). LCMS (ESI+): R_{f}=7.34, (M−H)+HRMS (ESI−): calculated for C₄₁H₄₃N₂O₁₁ m/z [M−H]⁻: 739.287234, observed 739.286311; ¹H NMR δ: 7.86 (dd, J=1.0 Hz, 2H), 7.62 (s, 2H), 7.52 (d, J=7.5 Hz, 2H), 5.37-5.82 (m, 2H), 3.70-3.89 (m, 2H), 3.37-3.56 (m, 1H), 3.18-3.29 (m, 3H), 1.79-2.10 (m, 2H), 1.48-1.74 (m, 4H), 1.05-1.45 (m, 24H).

### Example 128—Synthesis of 2-[(2S,3R)-2-amino-3-methoxybutanoyl]-5-{2-[(2S,3R)-2-amino-3-methoxybutanoyl]-1,3-dioxo-2,3-dihydro-1H-indene-5-carbonyl}-2,3-dihydro-1H-indene-1,3-dione

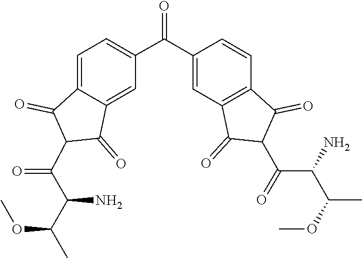 [SMILES: CO[C@@H](C)[C@@H](N)C(=O)C1C(=O)c2ccc(C(=O)c3ccc4c(c3)C(=O)C(C(=O)[C@@H](N)[C@@H](C)OC)C4=O)cc2C1=O]

In a similar manner to that of Example 111, tert-butyl N-[(2S,3R)-1-(5-{2-[(2S,3R)-2-{[(tert-butoxy)carbonyl]amino}-3-methoxybutanoyl]-1,3-dioxo-2,3-dihydro-1H-indene-5-carbonyl}-1,3-dioxo-2,3-dihydro-1H-inden-2-yl)-3-methoxy-1-oxobutan-2-yl]carbamate was prepared from 5,5′-carbonylbis(1H-indene-1,3(2H)-dione) (Example 4, 160.0 mg, 0.5 mmol) and Boc-O-methyl-L-threonine (CAS number 48068-25-3, 349.6 mg, 1.50 mmol). tert-Butyl N-[(2S,3R)-1-(5-{2-[(2S,3R)-2-{[(tert-butoxy)carbonyl]amino}-3-methoxybutanoyl]-1,3-dioxo-2,3-dihydro-1H-indene-5-carbonyl}-1,3-dioxo-2,3-dihydro-1H-inden-2-yl)-3-methoxy-1-oxobutan-2-yl]carbamate was subjected to deprotection conditions in the same manner as Example 116 to provide the title compound (78 mg, 30% yield). LCMS (ESI+): R_{f}=2.41, (M−H)+; HRMS (ESI−): calculated for C₂₉H₂₇N₂O₉ m/z [M−H]⁻: 547.172204, observed 547.170943; the compound would not form solutions with a variety of ¹H NMR solvents so the spectrum could not be taken.

EXAMPLE 129—tert-butyl (4R)-4-(5-{2-[(4R)-3-[(tert-butoxy)carbonyl]-1,3-thiazolidine-4-carbonyl]-1,3-dioxo-2,3-dihydro-1H-indene-5-carbonyl}-1,3-dioxo-2,3-dihydro-1H-indene-2-carbonyl)-1,3-thiazolidine-3-carboxylate

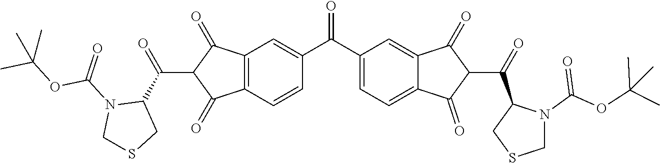

In a similar manner to that of Example 111, the title compound was prepared from 5,5′-carbonylbis(1H-indene-1,3(2H)-dione) (Example 4, 160.0 mg, 0.5 mmol) and (4R)-3-(tert-butoxycarbonyl)-1,3-thiazolidine-4-carboxylic acid (CAS number 51077-16-8, 349.6 mg, 1.50 mmol) to provide a dark solid bis-sodium salt (217 mg, 55% yield). LCMS (ESI+): Rf=6.64, (M−H)+; HRMS (ESI−): calculated for C₃₇H₃₅N₂O₁₁S₂ m/z [M−H]⁻: 747.168776, observed 747.170343; ¹H NMR δ: 7.87 (dd, J=7.5, 1.6 Hz, 2H), 7.65 (s, 2H), 7.56 (d, J=7.5 Hz, 2H), 5.65-5.70 (m, 2H), 4.60 (d, J=8.6 Hz, 2H), 4.33 (d, J=8.6 Hz, 2H), 3.44 (br d, J=10.0 Hz, 2H), 2.75-2.95 (m, 2H), 1.38 (s, 8H), 1.19-1.32 (s, 10H).

### Example 130—Synthesis of 4-{5-[2-(3-carboxypropanoyl)-1,3-dioxo-2,3-dihydro-1H-indene-5-carbonyl]-1,3-dioxo-2,3-dihydro-1H-inden-2-yl}-4-oxobutanoic acid

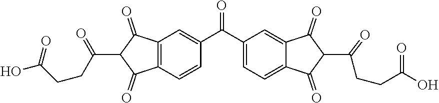 [SMILES: O=C(O)CCC(=O)C1C(=O)c2ccc(C(=O)c3ccc4c(c3)C(=O)C(C(=O)CCC(=O)O)C4=O)cc2C1=O]

In a similar manner to that of Example 111, methyl 4-{5-[2-(4-methoxy-4-oxobutanoyl)-1,3-dioxo-2,3-dihydro-1H-indene-5-carbonyl]-1,3-dioxo-2,3-dihydro-1H-inden-2-yl}-4-oxobutanoate was prepared from 5,5′-carbonylbis(1H-indene-1,3(2H)-dione) (Example 4, 160.0 mg, 0.5 mmol) and monomethyl succinate (CAS number 3878-55-5, 198.0 mg, 1.50 mmol) with a modified workup which included treatment with 15.0 ml of 2N HCl and filtration. To this isolated material there was added an excess of 1N NaOH 10.0 ml and a solution formed. The resulting solution was washed once with methylene chloride and filtered and treated with 1N HCl. The resulting solid was filtered to provide the title compound as a pink solid (217.2 mg, 55% yield). LCMS (ESI+): R_{f}=6.64, HRMS (ESI−): calculated for C₂₇H₁₇O₁₁ m/z [M−H]⁻: 517.077635, observed 517.076452; 1H NMR δ: 8.08 (dd, J=7.6, 1.2 Hz, 2H), 7.94 (s, 2H), 7.87 (d, J=7.7 Hz, 2H), 3.17 (t, J=7.1 Hz, 4H), 2.57 (t, J=7.1 Hz, 4H).

### Example 131—Synthesis of tert-butyl 6-[5-(2-{2-[(tert-butoxy)carbonyl]-2-azaspiro[3.3]heptane-6-carbonyl}-1,3-dioxo-2,3-dihydro-1H-indene-5-carbonyl)-1,3-dioxo-2,3-dihydro-1H-indene-2-carbonyl]-2-azaspiro[3.3]heptane-2-carboxylate

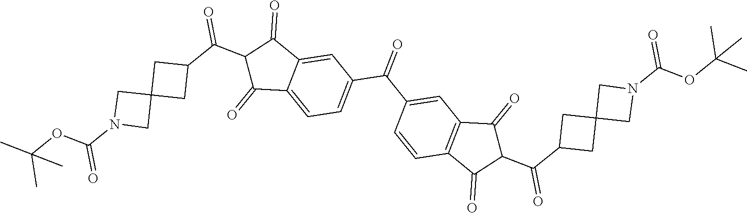 [SMILES: CC(C)(C)OC(=O)N1CC2(CC(C(=O)C3C(=O)c4ccc(C(=O)c5ccc6c(c5)C(=O)C(C(=O)C5CC7(C5)CN(C(=O)OC(C)(C)C)C7)C6=O)cc4C3=O)C2)C1]

In a similar manner to that of Example 111, the title compound was prepared from 5,5′-carbonylbis(1H-indene-1,3(2H)-dione) (Example 4, 160.0 mg, 0.5 mmol) and 2-(tert-butoxycarbonyl)-2-azaspiro[3.3]heptane-6-carboxylic acid (CAS number 1211526-53-2, 361.6 mg, 1.50 mmol) to provide a dark solid bis-sodium salt (298.2 mg, 75% yield). LCMS (ESI+): R_{f}=6.64, (M−H)+; HRMS (ESI−): calculated for C₄₃H₄₃N₂O₁₁ m/z [M−H]⁻: 763.2872, observed 763.2830; ¹H NMR δ: 7.84 (dd, J=1.0 Hz, 2H), 7.60 (s, 2H), 7.51 (d, J=7.6 Hz, 2H), 3.94-4.16 (m, 2H), 3.85 (br s, 4H), 3.68 (br s, 4H), 2.22 (br d, J=8.0 Hz, 8H), 1.34 (s, 18H).

### Example 132—Synthesis of tert-butyl (2S,4S)-2-(5-{2-[(2S,4S)-1-[(tert-butoxy) carbonyl]-4-methylpyrrolidine-2-carbonyl]-1,3-dioxo-2,3-dihydro-1H-indene-5-carbonyl}-1,3-dioxo-2,3-dihydro-1H-indene-2-carbonyl)-4-methylpyrrolidine-1-carboxylate

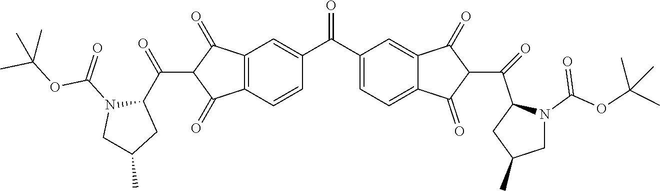 [SMILES: C[C@H]1C[C@@H](C(=O)C2C(=O)c3ccc(C(=O)c4ccc5c(c4)C(=O)C(C(=O)[C@@H]4C[C@H](C)CN4C(=O)OC(C)(C)C)C5=O)cc3C2=O)N(C(=O)OC(C)(C)C)C1]

In a similar manner to that of Example 111, the title compound was prepared from 5,5′-carbonylbis(1H-indene-1,3(2H)-dione) (Example 4, 160.0 mg, 0.5 mmol) and (2S,4S)-1-(tert-butoxycarbonyl)-4-methylpyrrolidine-2-carboxylic acid (CAS number 364750-81-2, 343.6 mg, 1.50 mmol) to provide a dark solid bis-sodium salt (289 mg, 74% yield). LCMS (ESI+): Rf=6.64, (M−H)+; HRMS (ESI−): calculated for C₄₁H₄₃N₂O₁₁ m/z [M−H]⁻: 739.2872, observed 739.2870; ¹H NMR δ: 7.86 (br d, J=7.7 Hz, 2H), 7.63 (s, 2H), 7.48-7.58 (m, 2H), 5.24-5.50 (m, 2H), 3.52-3.72 (m, 2H), 3.34 (s, 2H), 2.74 (s, 2H), 2.29-2.44 (m, 2H), 2.02-2.23 (m, 2H), 1.35 (s, 8H), 1.13-1.22 (m, 10H), 0.93 (t, J=1.0 Hz, 6H).

### Example 133—Synthesis of tert-butyl (2S)-2-(5-{2-[(2S)-1-[(tert-butoxy)carbonyl]-4-methylidenepyrrolidine-2-carbonyl]-1,3-dioxo-2,3-dihydro-1H-indene-5-carbonyl}-1,3-dioxo-2,3-dihydro-1H-indene-2-carbonyl)-4-methylidenepyrrolidine-1-carboxylate

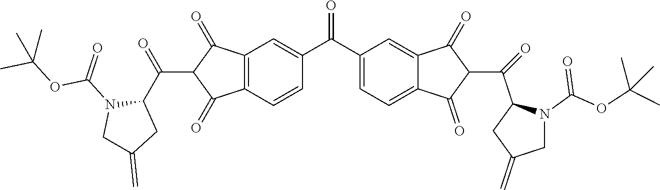 [SMILES: C=C1C[C@@H](C(=O)C2C(=O)c3ccc(C(=O)c4ccc5c(c4)C(=O)C(C(=O)[C@@H]4CC(=C)CN4C(=O)OC(C)(C)C)C5=O)cc3C2=O)N(C(=O)OC(C)(C)C)C1]

In a similar manner to that of Example 111, the title compound was prepared from 5,5′-carbonylbis(1H-indene-1,3(2H)-dione) (Example 4, 160.0 mg, 0.5 mmol) and (2S)-1-(tert-butoxycarbonyl)-4-methylidenepyrrolidine-2-carboxylic acid (CAS number 84348-38-9, 340.5 mg, 1.50 mmol) to provide a dark solid bis-sodium salt (302 mg, 78% yield). LCMS (ESI+): R_{f}=6.64, (M−H)+; HRMS (ESI−): calculated for C₄₁H₃₉N₂O₁₁ m/z [M−H]⁻: 735.2559, observed 735.2578; ¹H NMR δ: 7.88 (dd, J=1.0 Hz, 2H), 7.64 (s, 2H), 7.55 (d, J=7.5 Hz, 2H), 5.38-5.47 (m, 2H), 4.88 (br d, J=5.4 Hz, 4H), 3.90-4.00 (m, 4H), 2.91-3.03 (m, 2H), 2.27-2.38 (m, 2H), 1.38 (s, 8H), 1.23 (s, 10H).

### Example 134—Synthesis of propan-2-yl (2S)-2-(5-{1,3-dioxo-2-[(2S)-1-[(propan-2-yloxy)carbonyl]pyrrolidine-2-carbonyl]-2,3-dihydro-1H-indene-5-carbonyl}-1,3-dioxo-2,3-dihydro-1H-indene-2-carbonyl)pyrrolidine-1-carboxylate

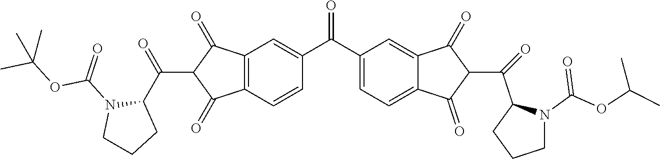 [SMILES: CC(C)OC(=O)N1CCC[C@H]1C(=O)C1C(=O)c2ccc(C(=O)c3ccc4c(c3)C(=O)C(C(=O)[C@@H]3CCCN3C(=O)OC(C)(C)C)C4=O)cc2C1=O]

In a similar manner to that of Example 111, except that the bis-sodium salt was not prepared, the title compound was prepared from 5,5′-carbonylbis(1H-indene-1,3(2H)-dione) (Example 4, 160.0 mg, 0.5 mmol) and (2S)-1-[(propan-2-yloxy)carbonyl]pyrrolidine-2-carboxylic acid (CAS number 1008530-34-4, 301.5 mg, 1.50 mmol) to provide a dark solid (220 mg, 60% yield). LCMS (ESI+): R_{f}=5.64, (M−H)+; HRMS (ESI−): calculated for C₃₇H₃₅N₂O₁₁ m/z [M−H]⁻: 683.2246, observed 683.2233; ¹H NMR δ: 7.90 (dd, J=1.0 Hz, 2H), 7.69 (s, 2H), 7.57 (d, J=1.0 Hz, 2H), 5.23-5.59 (m, 2H), 4.48-4.82 (m, 2H), 3.40-3.50 (m, 2H), 3.21-3.30 (m, 2H), 2.04-2.36 (m, 2H), 1.60-1.88 (m, 4H), 0.85-1.31 (m, 12H).

### Example 135—Synthesis of 5-{1,3-dioxo-2-[(1S,2R,6R,8R,9S)-4,4,11,11-tetramethyl-3,5,7,10,12-pentaoxatricyclo[7.3.0.0²,⁶]dodecane-8-carbonyl]-2,3-dihydro-1H-indene-5-carbonyl}-2-[(1S,2R,6R,8R,9S)-4,4,11,11-tetramethyl-3,5,7,10,12-pentaoxatricyclo[7.3.0.0²,⁶]dodecane-8-carbonyl]-2,3-dihydro-1H-indene-1,3-dione

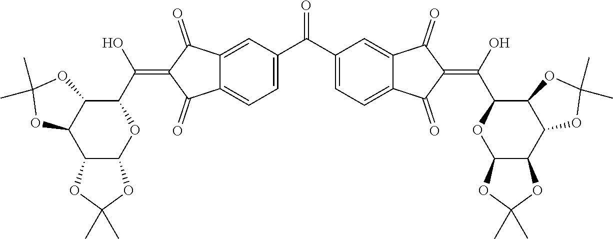

In a similar manner to that of Example 111, the title compound was prepared from 5,5′-carbonylbis(1H-indene-1,3(2H)-dione) (Example 4, 160.0 mg, 0.5 mmol) and 1,2:3,4-Di-O-isopropylidene-a-D-galacturonide (CAS number 25253-46-7, 411.0 mg, 1.50 mmol) to provide a dark solid bis-sodium salt (265.2 mg, 60% yield). LCMS (ESI+): R_{f}=6.64, (M−H)+; HRMS (ESI−): calculated for C₄₃H₄₁O₁₇ m/z [M−H]⁻: 829.234923, observed 829.235346; ¹H NMR δ: 7.87 (br d, J=7.6 Hz, 2H), 7.64 (s, 2H), 7.55 (d, J=7.5 Hz, 2H), 5.54 (d, J=5.1 Hz, 2H), 5.40-5.48 (m, 2H), 4.59 (qd, J=7.6, 2.3 Hz, 4H), 4.34 (dd, J=5.2, 2.1 Hz, 2H), 1.50 (s, 6H), 1.28 (d, J=6.7 Hz, 12H), 1.16 (s, 6H).

### Example 136—Synthesis of 2-[(2R)-2-methoxy-2-phenylacetyl]-5-({2-[(2R)-2-methoxy-2-phenylacetyl]-1,3-dioxo-2,3-dihydro-1H-inden-5-yl}sulfonyl)-2,3-dihydro-1H-indene-1,3-dione

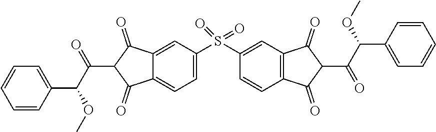 [SMILES: CO[C@@H](C(=O)C1C(=O)c2ccc(S(=O)(=O)c3ccc4c(c3)C(=O)C(C(=O)[C@H](OC)c3ccccc3)C4=O)cc2C1=O)c1ccccc1]

To a solution of dimethylaminopyridine (DMAP, 183 mg, 1.50 mmol), (2R)-methoxy(phenyl)acetic acid (CAS number 3966-32-3, 229.5 mg, 1.50 mmol) anhydrous DMF (9 ml) was added EDCI HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred overnight. The following day, 15.0 ml of 2N HCl was added and the resulting precipitant was filtered. The solid was washed once with 5 ml of 2N HCl and 5 ml of water. The dried solid was milled into a fine powder and was treated with 10 ml of 1N NaOH and 5 ml of methylene chloride. This biphasic suspension was shaken vigorously and filtered to provide the title compound dark solid bis-sodium salt (197.2 mg, 75%). LCMS (ESI+): R_{f}=5.67, (M−H)⁺=701.64; HRMS (ESI−): calculated for C₃₆H₂₅O₁₀S m/z [M−H]⁻: 649.1199, observed 649.1174; ¹H NMR δ: 8.06 (d, J=1.8 Hz, 2H), 7.75 (d, J=1.7 Hz, 2H), 7.55 (d, J=7.6 Hz, 2H), 7.34-7.44 (m, 4H), 7.18 (m, 6H), 6.14 (s, 2H), 3.18 (s, 6H).

### Example 137—Synthesis of 5-{[1,3-dioxo-2-(2-phenylacetyl)-2,3-dihydro-1H-inden-5-yl]sulfonyl}-2-(2-phenylacetyl)-2,3-dihydro-1H-indene-1,3-dione

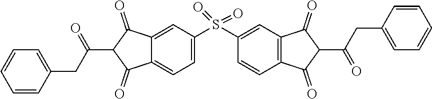 [SMILES: O=C(Cc1ccccc1)C1C(=O)c2ccc(S(=O)(=O)c3ccc4c(c3)C(=O)C(C(=O)Cc3ccccc3)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and phenylacetic acid (CAS number 103-82-2, 204.0 mg, 1.50 mmol) to provide a dark solid bis-sodium salt (261.0 mg, 82% yield). LCMS (ESI+): R_{f}=6.97, (M−H)⁺=701.64; HRMS (ESI−): calculated for C₃₄H₂₁O₈S m/z [M−H]⁻: 589.096262, observed 589.095774; ¹H NMR δ: 8.11 (br d, J=7.8 Hz, 2H), 7.78 (s, 2H), 7.58 (d, J=7.7 Hz, 2H), 7.09-7.26 (m, 10H), 4.07 (s, 4H).

### Example 138—Synthesis of tert-butyl (2S)-2-[5-({2-[(2S)-1-[(tert-butoxy)carbonyl]azetidine-2-carbonyl]-1,3-dioxo-2,3-dihydro-1H-inden-5-yl}sulfonyl)-1,3-dioxo-2,3-dihydro-1H-indene-2-carbonyl]azetidine-1-carboxylate

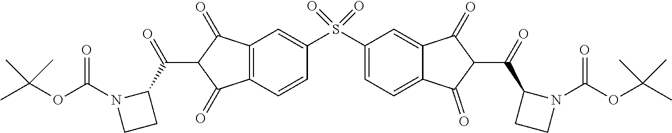 [SMILES: CC(C)(C)OC(=O)N1CC[C@H]1C(=O)C1C(=O)c2ccc(S(=O)(=O)c3ccc4c(c3)C(=O)C(C(=O)[C@@H]3CCN3C(=O)OC(C)(C)C)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and BOC-L-azetidine-2-carboxylic acid (CAS number 51077-14-6, 301.5 mg, 1.50 mmol) to provide a dark solid bis-sodium salt (212.0 mg, 55% yield). LCMS (ESI+): R_{f}=6.97, (M−H)+; HRMS (ESI−): calculated for C₃₆H₃₅N₂O₁₂S m/z [M−H]⁻: 719.191619, observed 719.190466; ¹H NMR δ: 7.86 (dd, J=7.5, 1.6 Hz, 2H), 7.63 (s, 2H), 7.55 (d, J=7.4 Hz, 2H), 5.38-5.71 (m, 2H), 3.59-3.90 (m, 4H), 1.61-1.87 (m, 4H), 1.18-1.32 (m, 18H).

### Example 139—Synthesis of 5-({1,3-dioxo-2-[2-(1H-pyrazol-1-yl)acetyl]-2,3-dihydro-1H-inden-5-yl}sulfonyl)-2-[2-(1H-pyrazol-1-yl)acetyl]-2,3-dihydro-1H-indene-1,3-dione

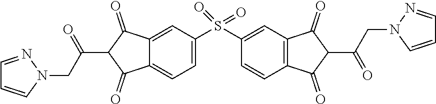 [SMILES: O=C(Cn1cccn1)C1C(=O)c2ccc(S(=O)(=O)c3ccc4c(c3)C(=O)C(C(=O)Cn3cccn3)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and 1H-pyrazol-1-ylacetic acid (CAS number 16034-48-3, 189.0 mg, 1.50 mmol) to provide a dark solid bis-sodium salt (30.2 mg, 9% yield). LCMS (ESI+): Rf=6.97, (M−H)+; HRMS (ESI−): calculated for C₂₈H₁₇N₄O₈S m/z [M−H]⁻: 569.077258, observed 569.075152; ¹H NMR δ: 7.92 (br d, J=7.6 Hz, 2H), 7.71 (s, 2H), 7.59-7.64 (m, 4H), 7.37 (s, 2H), 6.21 (s, 2H), 5.40 (s, 4H).

### Example 140—Synthesis of 2-(2-cyclopropylacetyl)-5-[2-(2-cyclopropylacetyl)-1,3-dioxo-2,3-dihydro-1H-indene-5-carbonyl]-2,3-dihydro-1H-indene-1,3-dione

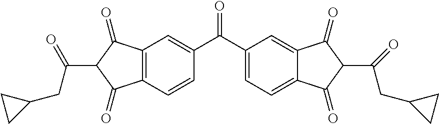 [SMILES: O=C(c1ccc2c(c1)C(=O)C(C(=O)CC1CC1)C2=O)c1ccc2c(c1)C(=O)C(C(=O)CC1CC1)C2=O]

In a similar manner to that of Example 111, except that the bis-sodium salt was not prepared, the title compound was prepared from 5,5′-carbonylbis(1H-indene-1,3(2H)-dione) (Example 4, 160.0 mg, 0.5 mmol) and cyclopropyl acetic acid (CAS number 5239-82-7, 150 mg, 1.5 mmol). The product was further heated in hot acetonitrile, filtered, and concentrated to afford the title compound as a dark solid (55 mg, 23%). LC/MS (ESI+): R_{f}=7.11 min, (M+H)⁺=483.77; HRMS (ESI−): calculated for C₂₉H₂₂O₇ m/z [M−H]⁻: 481.1293, observed 481.1286; ¹H NMR δ 8.11 (d, J=2.34 Hz, 3H), 7.87-8.02 (m, 4H), 3.47 (br. s., 5H), 2.77-2.91 (m, 5H), 1.00-1.15 (m, 2H), 0.46 (d, J=6.44 Hz, 4H), 0.24 (br. s., 4H).

### Example 141—Synthesis of 2-(2-cyclopentylacetyl)-5-[2-(2-cyclopentylacetyl)-1,3-dioxo-2,3-dihydro-1H-indene-5-carbonyl]-2,3-dihydro-1H-indene-1,3-dione

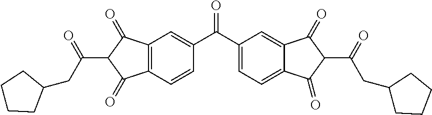 [SMILES: O=C(c1ccc2c(c1)C(=O)C(C(=O)CC1CCCC1)C2=O)c1ccc2c(c1)C(=O)C(C(=O)CC1CCCC1)C2=O]

In a similar manner to that of Example 111, except that the bis-sodium salt was not prepared, the title compound was prepared from 5,5′-carbonylbis(1H-indene-1,3(2H)-dione) (Example 4, 160.0 mg, 0.5 mmol) and cyclopentyl acetic acid (CAS number 1123-00-8, 265 μL, 1.5 mmol). The product was heated in hot acetonitrile, filtered, and concentrated. The resulting solid was washed with 1M HCl and methanol to afford the title compound as a dark solid (12 mg, 4%). LC/MS (ESI+): R_{f}=8.65 min, (M+H)⁺=539.81; HRMS (ESI−): calculated for C₃₃H₃₀O m/z [M−H]⁻: 537.1919, observed 537.1904; ¹H NMR δ 8.07 (d, J=8.10 Hz, 2H), 7.83-7.95 (m, 4H), 2.88-2.97 (m, 4H), 2.09-2.28 (m, 2H), 1.65-1.77 (m, 4H), 1.55-1.62 (m, 4H), 1.44-1.53 (m, 4H), 1.16-1.28 (m, 4H).

### Example 142—Synthesis of 2-(2-cyclopentylacetyl)-5-{[2-(2-cyclopentylacetyl)-1,3-dioxo-2,3-dihydro-1H-inden-5-yl]sulfonyl}-2,3-dihydro-1H-indene-1,3-dione

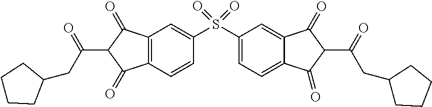 [SMILES: O=C(CC1CCCC1)C1C(=O)c2ccc(S(=O)(=O)c3ccc4c(c3)C(=O)C(C(=O)CC3CCCC3)C4=O)cc2C1=O]

In a similar manner to that of Example 136, except that the bis-sodium salt was not prepared, the title compound was prepared from 5,5′-sulfonylbis(1H-indene-1,3(2H)-dione) (Example 12, 177 mg, 0.5 mmol) and cyclopentyl acetic acid (CAS number 1123-00-8, 265 μL, 1.5 mmol). The product was heated in hot acetonitrile, filtered, and concentrated. The resulting solid was washed with 1M HCl and methanol to afford the title compound as a dark solid (59 mg, 21%). LC/MS (ESI+): R_{f}=8.26 min, (M+H)⁺=575.78; HRMS (ESI−): calculated for C₃₂H₃₀O₈ m/z [M−H]⁻: 573.1589, observed 573.1578; ¹H NMR δ 8.42 (d, J=7.60 Hz, 2H), 8.22 (s, 2H), 7.91 (d, J=7.00 Hz, 2H), 2.85-2.94 (m, 4H), 2.11-2.26 (m, 2H), 1.36-1.75 (m, 12H), 1.09-1.27 (m, 4H).

### Example 143—Synthesis of 5,5-[1,3-dioxo-2-(2-phenoxyacetyl)-2,3-dihydro-1H-indene-5-carbonyl]-2-(2-phenoxyacetyl)-2,3-dihydro-1H-indene-1,3-dione

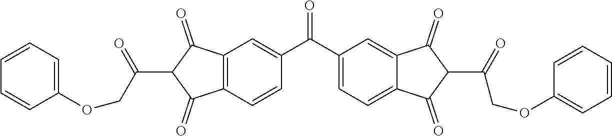 [SMILES: O=C(c1ccc2c(c1)C(=O)C(C(=O)COc1ccccc1)C2=O)c1ccc2c(c1)C(=O)C(C(=O)COc1ccccc1)C2=O]

In a similar manner to that of Example 111, the title compound was prepared from 5,5′-carbonylbis(1H-indene-1,3(2H)-dione) (Example 4, 160.0 mg, 0.5 mmol) and phenoxyacetic acid (CAS number 122-59-8,3 equivalents) as a dark colored sodium salt (26 mg, 8%). LC/MS (ESI+): R_{f}=6.88 min, (M+H)⁺=587.75; HRMS (ESI−): calculated for C₃₅H₂₂O₉ m/z [M−H]⁻: 585.1191, observed 585.1179; ¹H NMR δ 7.87 (d, J=7.03 Hz, 2H), 7.66 (s, 2H), 7.55 (d, J=7.62 Hz, 2H), 7.22 (t, J=7.62 Hz, 4H), 6.77-6.89 (m, 6H), 5.09 (s, 4H).

### Example 144—Synthesis of FC-8396 5-{[1,3-dioxo-2-(2-phenoxyacetyl)-2,3-dihydro-1H-inden-5-yl]sulfonyl}-2-(2-phenoxyacetyl)-2,3-dihydro-1H-indene-1,3-dione

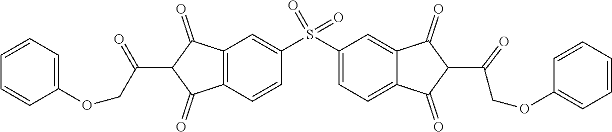 [SMILES: O=C(COc1ccccc1)C1C(=O)c2ccc(S(=O)(=O)c3ccc4c(c3)C(=O)C(C(=O)COc3ccccc3)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and phenoxyacetic acid (CAS number 122-59-8, 157 μL, 1.5 mmol) as a dark colored sodium salt (91 mg, 84%). LC/MS (ESI+): R_{f}=6.18 min, (M+H)⁺=623.73; HRMS (ESI−): calculated for C₃₄H₂₂O₁₀S m/z [M−H]⁻: 621.0861, observed 621.0870; ¹H NMR δ 8.14 (d, J=7.62 Hz, 3H), 7.81 (s, 2H), 7.62 (d, J=7.62 Hz, 2H), 7.22 (t, J=7.62 Hz, 5H), 6.77-6.89 (m, 6H), 5.09 (s, 4H).

### Example 145—Synthesis of 2-(3-methylbutanoyl)-5-{[2-(3-methylbutanoyl)-1,3-dioxo-2,3-dihydro-1H-inden-5-yl]sulfonyl}-2,3-dihydro-1H-indene-1,3-dione

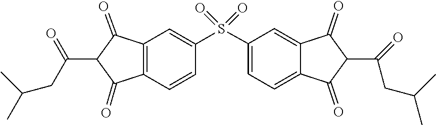 [SMILES: CC(C)CC(=O)C1C(=O)c2ccc(S(=O)(=O)c3ccc4c(c3)C(=O)C(C(=O)CC(C)C)C4=O)cc2C1=O]

In a similar manner to that of Example 136, except that the bis-sodium salt was not prepared, the title compound was prepared from 5,5′-sulfonylbis(1H-indene-1,3(2H)-dione) (Example 12, 177 mg, 0.5 mmol) and isovaleric acid (CAS number 503-74-2, 106 μL, 1.5 mmol). The product was purified with a C18 reverse phase column using acetonitrile/water and 0.1% ammonium hydroxide modifier to afford the title compound (46 mg, 18%). LC/MS (ESI+): R_{f}=7.46 min, (M+H)⁺=523.62; HRMS (ESI−): calculated for C₂₈H₂O₈S m/z [M−H]⁻521.1276, observed 521.1295; ¹H NMR δ 8.41 (d, J=6.44 Hz, 2H), 8.14 (s, 2H), 7.79 (d, J=7.60 Hz, 2H), 2.77 (d, J=7.00 Hz, 4H), 1.95-2.19 (m, 2H), 0.91 (d, J=6.40 Hz, 12H).

### Example 146—Synthesis of 5-[1,3-dioxo-2-(oxolane-3-carbonyl)-2,3-dihydro-1H-indene-5-carbonyl]-2-(oxolane-3-carbonyl)-2,3-dihydro-1H-indene-1,3-dione

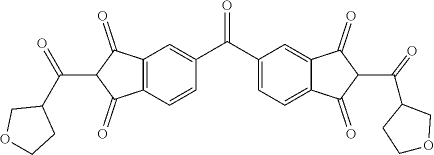 [SMILES: O=C(c1ccc2c(c1)C(=O)C(C(=O)C1CCOC1)C2=O)c1ccc2c(c1)C(=O)C(C(=O)C1CCOC1)C2=O]

In a similar manner to that of Example 111, except that the bis-sodium salt was not prepared, the title compound was prepared from 5,5′-carbonylbis(1H-indene-1,3(2H)-dione) (Example 4, 160.0 mg, 0.5 mmol) and tetrahyo-furan-3-carboxylic acid (CAS number 89364-31-8,3 equivalents). The product was purified with a C18 reverse phase column using acetonitrile/water and 0.1% ammonium hydroxide modifier to afford the title compound (48 mg, 19%). LC/MS (ESI+): R_{f}=5.53 min, (M+H)⁺=515.60; HRMS (ESI−): calculated for C₂₉H₂₂O₉ m/z [M−H]⁻: 513.1191, observed 513.1197; ¹H NMR δ 8.03 (d, J=7.00 Hz, 4H), 7.78-7.93 (m, 4H), 4.18-4.40 (m, 4H), 3.66-3.85 (m, 7H), 2.75 (d, J=45.10 Hz, 2H), 2.01-2.13 (m, 4H).

### Example 147—Synthesis of 5-{[1,3-dioxo-2-(oxolane-3-carbonyl)-2,3-dihydro-1H-inden-5-yl]sulfonyl}-2-(oxolane-3-carbonyl)-2,3-dihydro-1H-indene-1,3-dione

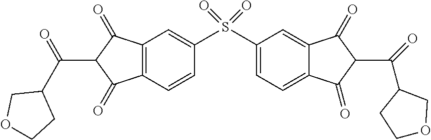 [SMILES: O=C1c2ccc(S(=O)(=O)c3ccc4c(c3)C(=O)C(C(=O)C3CCOC3)C4=O)cc2C(=O)C1C(=O)C1CCOC1]

In a similar manner to that of Example 136, except that the bis-sodium salt was not prepared, the title compound was prepared from 5,5′-sulfonylbis(1H-indene-1,3(2H)-dione) (Example 12, 177 mg, 0.5 mmol) and tetrahyo-furan-3-carboxylic acid (CAS number 89364-31-8, 287 μL, 1.5 mmol). The product was purified with a C18 reverse phase column using acetonitrile/water and 0.1% ammonium hydroxide modifier to afford the title compound (162 mg, 59%). LC/MS (ESI+): R_{f}=4.84 min, (M+H)⁺=551.64; HRMS (ESI−): calculated for C₂₈H₂₂O₁₀S m/z [M−H]⁻: 549.0861, observed 549.0831; ¹H NMR δ 8.04-8.21 (m, 2H), 7.72-7.91 (m, 2H), 7.47-7.67 (m, 2H), 3.98-4.34 (m, 2H), 3.74-3.95 (m, 2H), 3.63-3.79 (m, 6H), 1.77-2.12 (m, 2H).

### Example 148—Synthesis of 2-(2-cyclohexylacetyl)-5-{[2-(2-cyclohexylacetyl)-1,3-dioxo-2,3-dihydro-1H-inden-5-yl]sulfonyl}-2,3-dihydro-1H-indene-1,3-dione

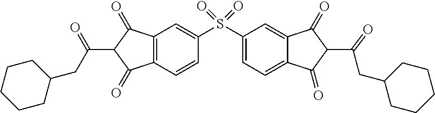 [SMILES: O=C(CC1CCCCC1)C1C(=O)c2ccc(S(=O)(=O)c3ccc4c(c3)C(=O)C(C(=O)CC3CCCCC3)C4=O)cc2C1=O]

In a similar manner to that of Example 136, except that the bis-sodium salt was not prepared, the title compound was prepared from 5,5′-sulfonylbis(1H-indene-1,3(2H)-dione) (Example 12, 177 mg, 0.5 mmol) and cyclohexane acetic acid (CAS number 5292-21-7, 212 μL, 1.5 mmol). The product was purified with a C18 reverse phase column using acetonitrile/water and 0.1% ammonium hydroxide modifier. The corresponding fractions were dried and the resulting compound was washed with acetonitrile/water and filtered to afford the title compound (58.4 mg, 19%). LC/MS (ESI+): R_{f}=8.95 min, (M+H)⁺=603.73; HRMS (ESI−): calculated for C₃₄H₃₄O₈S m/z [M−H]⁻: 601.1902, observed 601.1912; ¹H NMR δ 8.37 (d, J=7.60 Hz, 2H), 8.11 (s, 2H), 7.83 (d, J=7.60 Hz, 2H), 2.71-2.79 (m, 4H), 1.53-1.76 (m, 12H), 1.11-1.25 (m, 6H), 0.80-1.08 (m, 4H).

### Example 149—Synthesis of 2-(2-cyclopropylacetyl)-5-{[2-(2-cyclopropylacetyl)-1,3-dioxo-2,3-dihydro-1H-inden-5-yl]sulfonyl}-2,3-dihydro-1H-indene-1,3-dione

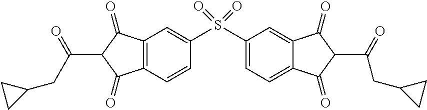 [SMILES: O=C(CC1CC1)C1C(=O)c2ccc(S(=O)(=O)c3ccc4c(c3)C(=O)C(C(=O)CC3CC3)C4=O)cc2C1=O]

In a similar manner to that of Example 136, except that the bis-sodium salt was not prepared, the title compound was prepared from 5,5′-sulfonylbis(1H-indene-1,3(2H)-dione) (Example 12, 177 mg, 0.5 mmol) and cyclopropane acetic acid (CAS Number 5239-82-7, 140 μL, 1.5 mmol). The product was purified with a C18 reverse phase column using acetonitrile/water and 0.1% ammonium hydroxide modifier. The corresponding fractions were dried and the resulting compound was washed with acetonitrile/1N HCl and filtered to afford the title compound (46 mg, 18%). LC/MS (ESI+): R_{f}=6.85 min, (M+H)⁺=519.61; HRMS (ES−): calculated for C₂₈H₂₂O₈S m/z [M−H]⁻: 517.0963, observed 517.0963; ¹H NMR δ 8.29 (d, J=5.90 Hz, qH), 8.05 (s, 2H), 7.75-7.84 (m, 2H), 2.67-2.79 (m, 4H), 0.94-1.05 (m, 2H), 0.38 (d, J=5.30 Hz, 4H), 0.15 (br. s., 4H).

### Example 150—Synthesis of 2-butanoyl-5-(2-butanoyl-1,3-dioxo-2,3-dihydro-1H-indene-5-carbonyl)-2,3-dihydro-1H-indene-1,3-dione

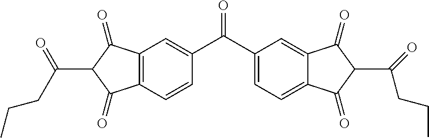 [SMILES: CCCC(=O)C1C(=O)c2ccc(C(=O)c3ccc4c(c3)C(=O)C(C(=O)CCC)C4=O)cc2C1=O]

In a similar manner to that of Example 111, except that the bis-sodium salt was not prepared, the title compound was prepared from 5,5′-carbonylbis(1H-indene-1,3(2H)-dione) (Example 4, 160.0 mg, 0.5 mmol) and butanoic acid (CAS number 107-92-6, 138 μL, 1.5 mmol). The resulting solid was washed with ACN:1N HCl (20:80) and filtered. The filtrate was then washed with ACN and filtered to afford the title compound (15 mg, 7%). LC/MS (ESI+): R_{f}=7.18 min, (M+H)⁺=459.14; HRMS (ESI−): calculated for C₂₇H₂₂O₇ m/z [M−H]⁻: 457.1293, observed 457.1315; ¹H NMR (pyridine-d₅) δ 8.37 (s, 2H), 7.99 (d, J=7.62 Hz, 2H), 7.88 (d, J=7.03 Hz, 2H), 3.43 (t, J=7.32 Hz, 4H), 2.72 (s, 2H), 1.94 (q, J=7.60 Hz, 4H), 1.03 (t, J=7.32 Hz, 6H).

### Example 151—Synthesis of 2-(4-methoxycyclohexanecarbonyl)-5-{[2-(4-methoxycyclohexanecarbonyl)-1,3-dioxo-2,3-dihydro-1H-inden-5-yl]sulfonyl}-2,3-dihydro-1H-indene-1,3-dione

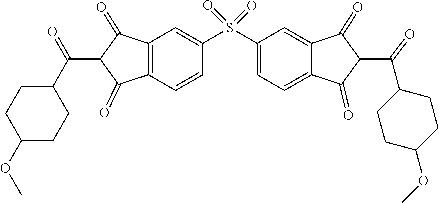 [SMILES: COC1CCC(C(=O)C2C(=O)c3ccc(S(=O)(=O)c4ccc5c(c4)C(=O)C(C(=O)C4CCC(OC)CC4)C5=O)cc3C2=O)CC1]

In a similar manner to that of Example 136, except that the bis-sodium salt was not prepared, the title compound was prepared from 5,5′-sulfonylbis(1H-indene-1,3(2H)-dione) (Example 12, 177 mg, 0.5 mmol) and 4-methoxycyclohexanecarboxylic acid (CAS number 95233-12-8, 236 μL, 1.5 mmol). The resulting solid was washed with ACN:1N HCl (20:80) and filtered to afford the title compound (41 mg, 13%). LC/MS (ESI+): R_{f}=6.89 min, purity=84%, (M+H)⁺=635.01; HRMS (ESI−): calculated for C₃₄H₃₄O₁₀S m/z [M−H]⁻: 633.1800, observed 633.1794; ¹H NMR δ 8.23-8.35 (m, 2H), 7.98-8.14 (m, 2H), 7.73-7.84 (m, 2H), 3.15-3.24 (m, 6H), 3.02-3.10 (m, 2H), 1.98-2.08 (m, 4H), 1.64-1.78 (m, 4H), 1.31-1.46 (m, 4H), 1.04-1.19 (m, 4H).

### Example 152—Synthesis of 2-(4-methoxycyclohexanecarbonyl)-5-{[2-(4-methoxycyclohexanecarbonyl)-1,3-dioxo-2,3-dihydro-1H-inden-5-yl]sulfonyl}-2,3-dihydro-1H-indene-1,3-dione

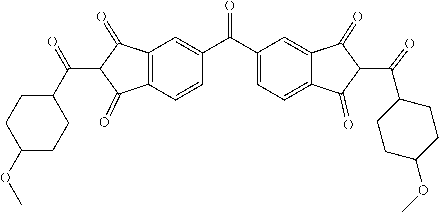 [SMILES: COC1CCC(C(=O)C2C(=O)c3ccc(C(=O)c4ccc5c(c4)C(=O)C(C(=O)C4CCC(OC)CC4)C5=O)cc3C2=O)CC1]

In a similar manner to that of Example 111, except that the bis-sodium salt was not prepared, the title compound was prepared from 5,5′-carbonylbis(1H-indene-1,3(2H)-dione) (Example 4, 160.0 mg, 0.5 mmol) and 4-methoxycyclohexanecarboxylic acid (CAS number 95233-12-8, 236 μL, 1.5 mmol). The resulting crude was washed with ACN:1N HCl (20:80) and filtered to afford the title compound (29 mg, 10%). LC/MS (ESI+): R_{f}=7.29 min, purity=91%, (M+H)⁺=599.79; HRMS (ESI−): calculated for C₃₅H₃₄O₉ m/z [M−H]⁻: 597.2130, observed 597.2112; ¹H NMR δ 8.05 (d, J=8.80 Hz, 2H), 7.81-7.96 (m, 4H), 3.23 (t, J=5.90 Hz, 6H), 3.06-3.17 (m, 2H), 2.02-2.14 (m, 4H), 1.76-1.86 (m, 4H), 1.40-1.55 (m, 4H), 1.08-1.23 (m, 4H).

### Example 153—Synthesis of 5-(1,3-dioxo-2-pentanoyl-2,3-dihydro-1H-indene-5-carbonyl)-2-pentanoyl-2,3-dihydro-1H-indene-1,3-dione

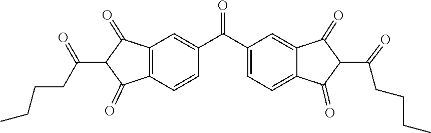 [SMILES: CCCCC(=O)C1C(=O)c2ccc(C(=O)c3ccc4c(c3)C(=O)C(C(=O)CCCC)C4=O)cc2C1=O]

In a similar manner to that of Example 111, except that the bis-sodium salt was not prepared, the title compound was prepared from 5,5′-carbonylbis(1H-indene-1,3(2H)-dione) (Example 4,160.0 mg, 0.5 mmol) and valeric acid (CAS number 109-52-4, 165 μL, 1.5 mmol). The product was purified with a C18 reverse phase column using acetonitrile/water and 0.1% ammonium hydroxide modifier. The corresponding fractions were dried and the resulting compound was washed with acetonitrile/1N HCl and filtered to afford the title compound (25 mg, 10%). LC/MS (ESI+): R_{f}=7.70 min, purity=71%, (M+H)⁺=487.51; HRMS (ESI−): calculated for C₂₉H₂₆O₇ m/z [M−H]⁻: 485.1606, observed 485.1603; ¹H NMR δ 8.09 (d, J=7.03 Hz, 2H), 7.86-8.00 (m, 4H), 3.15-3.29 (m, 4H), 1.50-1.65 (m, 4H), 1.27-1.43 (m, 4H), 0.87 (t, J=7.00 Hz, 6H).

### Example 154—Synthesis 5-[(1,3-dioxo-2-pentanoyl-2,3-dihydro-1H-inden-5-yl)sulfonyl]-2-pentanoyl-2,3-dihydro-1H-indene-1,3-dione

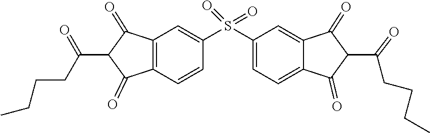 [SMILES: CCCCC(=O)C1C(=O)c2ccc(S(=O)(=O)c3ccc4c(c3)C(=O)C(C(=O)CCCC)C4=O)cc2C1=O]

In a similar manner to that of Example 136, except that the bis-sodium salt was not prepared, the title compound was prepared from 5,5′-sulfonylbis(1H-indene-1,3(2H)-dione) (Example 12, 177 mg, 0.5 mmol) and valeric acid (CAS number 109-52-4, 165 μL, 1.5 mmol). The product was purified with a C18 reverse phase column using acetonitrile/water and 0.1% ammonium hydroxide modifier. The corresponding fractions were dried and the resulting compound was washed with acetonitrile/1 N HCl and filtered to afford the title compound (40 mg, 15%). LC/MS (ESI+): R_{f}=7.51 min, purity=87%, (M+H)⁺=523.62; HRMS (ESI−): calculated for C₂₈H₂₆O₈S m/z [M−H]⁻: 521.1276, observed 521.1268; ¹H NMR δ 8.34 (d, J=8.20 Hz, 2H), 8.13 (s, 2H), 7.85 (d, J=7.62 Hz, 2H), 2.86 (t, J=7.32 Hz, 4H), 1.45-1.59 (m, 4H), 1.24-1.38 (m, 4H), 0.86 (t, J=7.32 Hz, 6H).

### Example 155—Synthesis of tert-butyl N-[(2S)-1-(5-{2-[(2S)-2-{[(tert-butoxy)carbonyl](methyl)amino}propanoyl]-1,3-dioxo-2,3-dihydro-1H-indene-5-carbonyl}-1,3-dioxo-2,3-dihydro-1H-inden-2-yl)-1-oxopropan-2-yl]-N-methylcarbamate

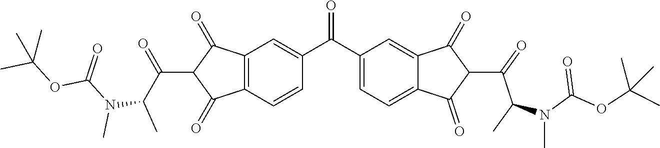 [SMILES: C[C@@H](C(=O)C1C(=O)c2ccc(C(=O)c3ccc4c(c3)C(=O)C(C(=O)[C@H](C)N(C)C(=O)OC(C)(C)C)C4=O)cc2C1=O)N(C)C(=O)OC(C)(C)C]

In a similar manner to that of Example 111, the title compound was prepared from 5,5′-carbonylbis(1H-indene-1,3(2H)-dione) (Example 4, 160.0 mg, 0.5 mmol) and Boc-N-methyl-L-alanine (CAS number 16948-16-6, 305 μL, 1.5 mmol) as a dark colored sodium salt (299 mg, 87%). LC/MS (ESI+): R_{f}=6.98 min, (M+H−(Boc)₂)⁺=489.55; HRMS (ESI−): calculated for C₃₇H₄₀N₂O₁₁ m/z [M−H]⁻: 687.2559, observed 687.2556; ¹H NMR δ 8.51 (s, 2H), 7.83 (d, J=7.03 Hz, 2H), 7.62 (s, 2H), 7.50 (d, J=7.00 Hz, 2H), 5.46-5.72 (m, 2H), 2.84 (br. s, 6H), 1.36 (br. s., 6H), 1.25 (br. s., 18H).

### Example 156—Synthesis of tert-butyl N-[(2S)-1-[5-({2-[(2S)-2-{[(tert-butoxy)carbonyl](methyl)amino}propanoyl]-1,3-dioxo-2,3-dihydro-1H-inden-5-yl}sulfonyl)-1,3-dioxo-2,3-dihydro-1H-inden-2-yl]-1-oxopropan-2-yl]-N-methylcarbamate

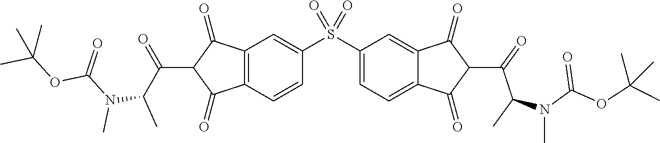 [SMILES: C[C@@H](C(=O)C1C(=O)c2ccc(S(=O)(=O)c3ccc4c(c3)C(=O)C(C(=O)[C@H](C)N(C)C(=O)OC(C)(C)C)C4=O)cc2C1=O)N(C)C(=O)OC(C)(C)C]

In a similar manner to that of Example 136, the title compound was prepared from 5,5′-sulfonylbis(1H-indene-1,3(2H)-dione) (Example 12, 177 mg, 0.5 mmol) and Boc-N-methyl-L-alanine (CAS number 16948-16-6, 305 μL, 1.5 mmol) as a dark colored sodium salt (285 mg, 76%). LC/MS (ESI+): R_{f}=7.26 min, (M+H−(Boc)₂)⁺=612.66; HRMS (ESI−): calculated for C₃₆H₄₀N₂O₁₂S m/z [M−H]⁻: 723.2229, observed 723.2256; ¹H NMR δ 8.52 (s, 2H), 8.07 (d, J=7.60 Hz, 2H), 7.74 (s, 2H), 7.54 (d, J=8.20 Hz, 2H), 5.43-5.65 (m, 2H), 2.83 (t, J=36.30 Hz, 6H), 1.32 (s, 6H), 1.22 (s, 18H).

### Example 157—Synthesis of tert-butyl N-[(2S)-1-(5-{2-[(2S)-2-{[(tert-butoxy)carbonyl]amino}-3-phenylpropanoyl]-1,3-dioxo-2,3-dihydro-1H-indene-5-carbonyl}-1,3-dioxo-2,3-dihydro-1H-inden-2-yl)-1-oxo-3-phenylpropan-2-yl]carbamate

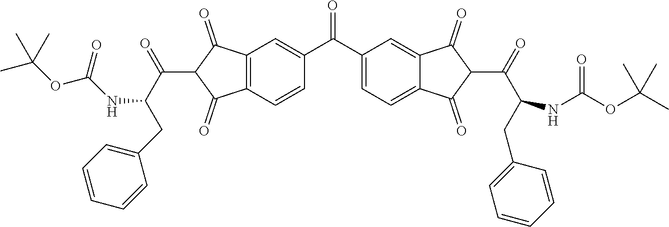 [SMILES: CC(C)(C)OC(=O)N[C@@H](Cc1ccccc1)C(=O)C1C(=O)c2ccc(C(=O)c3ccc4c(c3)C(=O)C(C(=O)[C@H](Cc3ccccc3)NC(=O)OC(C)(C)C)C4=O)cc2C1=O]

In a similar manner to that of Example 111, the title compound was prepared from 5,5′-carbonylbis(1H-indene-1,3(2H)-dione) (Example 4, 160.0 mg, 0.5 mmol) and Boc-N-L-phenylalanine (CAS number 37553-65-4, 398 μL, 1.5 mmol) as a dark colored sodium salt (173 mg, 43%). LC/MS (ESI+): R_{f}=7.26 min, (M+H−(Boc)₂)⁺=613.66; HRMS (ESI−): calculated for C₃₇H₄₀N₂O₁₁ m/z [M−H]⁻: 811.2872, observed 811.2881; ¹H NMR δ 7.89 (d, J=6.40 Hz, 2H), 7.70 (s, 2H), 7.60 (d, J=7.62 Hz, 2H), 7.37 (d, J=6.40 Hz, 4H), 7.22 (t, J=7.60 Hz, 4H), 7.14 (d, J=7.03 Hz, 2H), 5.13-5.42 (m, 2H), 3.32 (s, 6H), 2.84-3.15 (m, 2H), 1.27 (s, 14H), 1.09 (br. s., 4H).

### Example 158—Synthesis of tert-butyl N-[(2S)-1-[5-({2-[(2S)-2-{[(tert-butoxy)carbonyl]amino}-3-phenylpropanoyl]-1,3-dioxo-2,3-dihydro-1H-inden-5-yl}sulfonyl)-1,3-dioxo-2,3-dihydro-1H-inden-2-yl]-1-oxo-3-phenylpropan-2-yl]carbamate

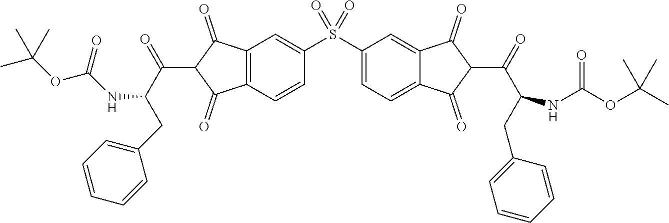 [SMILES: CC(C)(C)OC(=O)N[C@@H](Cc1ccccc1)C(=O)C1C(=O)c2ccc(S(=O)(=O)c3ccc4c(c3)C(=O)C(C(=O)[C@H](Cc3ccccc3)NC(=O)OC(C)(C)C)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and Boc-N-L-phenylalanine (CAS number 37553-65-4, 398 μL, 1.5 mmol) as a dark colored sodium salt (177 mg, 42%). LC/MS (ESI+): R_{f}=7.31 min, (M+H−(Boc)₂)⁺=649.64; HRMS (ESI−): calculated for C₄₆H₄₄N₂O₁₂S m/z [M−H]⁻: 847.2542, observed 847.2543; ¹H NMR δ 8.13 (d, J=7.60 Hz, 2H), 7.84 (s, 2H), 7.62 (d, J=7.00 Hz, 2H), 7.30-7.45 (m, 4H), 7.18-7.26 (m, 4H), 7.11-7.17 (m, 2H), 6.17-6.30 (m, 2H), 5.13-5.36 (m, 2H), 2.92 (t, J=12.90 Hz, 4H), 1.26 (br. s, 14H), 1.07 (br. s, 4H).

### Example 159—Synthesis of benzyl 3-[5-(2-{1-[(benzyloxy)carbonyl]pyrrolidine-3-carbonyl}-1,3-dioxo-2,3-dihydro-1H-indene-5-carbonyl)-1,3-dioxo-2,3-dihydro-1H-indene-2-carbonyl]pyrrolidine-1-carboxylate

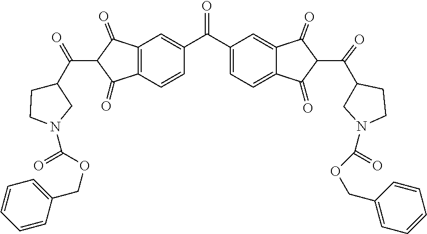 [SMILES: O=C(c1ccc2c(c1)C(=O)C(C(=O)C1CCN(C(=O)OCc3ccccc3)C1)C2=O)c1ccc2c(c1)C(=O)C(C(=O)C1CCN(C(=O)OCc3ccccc3)C1)C2=O]

In a similar manner to that of Example 111, the title compound was prepared from 5,5′-carbonylbis(1H-indene-1,3(2H)-dione) (Example 4, 160.0 mg, 0.5 mmol) and 1-N-Cbz-pyrrolidine-3-carboxylic acid (CAS number 188527-21-1, 374 μL, 1.5 mmol) as a dark colored sodium salt (296 mg, 76%). LC/MS (ESI+): R_{f}=6.93 min, (M+H)⁺=781.70; HRMS (ESI−): calculated for C₄₅H₃₆N₂O₁₁ m/z [M−H]⁻: 779.2246, observed 779.2238; ¹H NMR δ 7.93 (s, 2H), 7.85 (d, J=8.20 Hz, 2H), 7.63 (s, 2H), 7.54 (d, J=7.60 Hz, 2H), 7.33 (d, J=2.90 Hz, 10H), 5.03 (s, 4H), 4.03-4.30 (m, 2H), 1.82-2.07 (m, 4H).

### Example 160—Synthesis of benzyl 3-{5-[(2-{1-[(benzyloxy)carbonyl]pyrrolidine-3-carbonyl}-1,3-dioxo-2,3-dihydro-1H-inden-5-yl)sulfonyl]-1,3-dioxo-2,3-dihydro-1H-indene-2-carbonyl}pyrrolidine-1-carboxylate

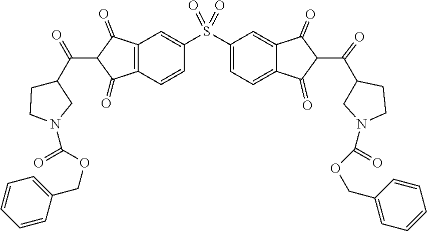 [SMILES: O=C1c2ccc(S(=O)(=O)c3ccc4c(c3)C(=O)C(C(=O)C3CCN(C(=O)OCc5ccccc5)C3)C4=O)cc2C(=O)C1C(=O)C1CCN(C(=O)OCc2ccccc2)C1]

In a similar manner to that of Example 136, the title compound was prepared from 5,5′-sulfonylbis(1H-indene-1,3(2H)-dione) (Example 12, 177 mg, 0.5 mmol) and 1-N-Cbz-pyrrolidine-3-carboxylic acid (CAS number 188527-21-1, 374 μL, 1.5 mmol) as a dark colored sodium salt (353 mg, 87%). LC/MS (ESI+): R_{f}=6.57 min, (M+H)⁺=817.68; HRMS (ESI−): calculated for C₄₄H₃₆N₂O₁₂S m/z [M−H]⁻: 815.1916, observed 815.1923; ¹H NMR δ 8.08 (d, J=8.20 Hz, 2H), 7.75 (s, 2H), 7.55 (d, J=7.00 Hz, 2H), 7.31 (d, J=6.40 Hz, 10H), 5.01 (s, 4H), 3.97-4.19 (m, 2H), 1.80-2.07 (m, 4H).

### Example 161—Synthesis of tert-butyl (2R)-2-(5-{2-[(3R)-1-[(tert-butoxy)carbonyl]pyrrolidine-3-carbonyl]-1,3-dioxo-2,3-dihydro-1H-indene-5-carbonyl}-1,3-dioxo-2,3-dihydro-1H-indene-2-carbonyl)pyrrolidine-1-carboxylate

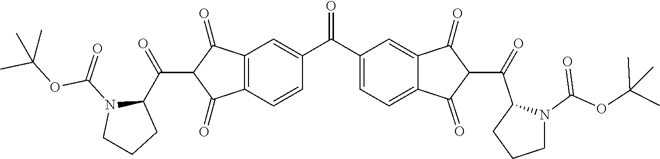 [SMILES: CC(C)(C)OC(=O)N1CCC[C@@H]1C(=O)C1C(=O)c2ccc(C(=O)c3ccc4c(c3)C(=O)C(C(=O)[C@H]3CCCN3C(=O)OC(C)(C)C)C4=O)cc2C1=O]

In a similar manner to that of Example 111, the title compound was prepared from 5,5′-carbonylbis(1H-indene-1,3(2H)-dione) (Example 4, 160.0 mg, 0.5 mmol) and N-Boc-D-Proline (CAS number 37784-17-1, 323 mg, 1.5 mmol) as a dark colored sodium salt (174 mg, 49%). LC/MS (ESI+): R_{f}=7.20 min, (M+H+Na)⁺=735.87; HRMS (ESI−): calculated for C₃₉H₄₀N₂O₁₁ m/z [M−H]⁻: 711.2559, observed 711.2541; ¹H NMR δ 7.84 (d, J=7.00 Hz, 2H), 7.62 (s, 2H), 7.47-7.56 (m, 2H), 5.21-5.46 (m, 2H), 1.95-2.24 (m, 2H), 1.53-1.83 (m, 4H), 1.35 (s, 4H), 1.20 (s, 14H).

### Example 162—Synthesis of tert-butyl (2R)-2-[5-({2-[(2R)-1-[(tert-butoxy)carbonyl]pyrrolidine-2-carbonyl]-1,3-dioxo-2,3-dihydro-1H-inden-5-yl}sulfonyl)-1,3-dioxo-2,3-dihydro-1H-indene-2-carbonyl]pyrrolidine-1-carboxylate

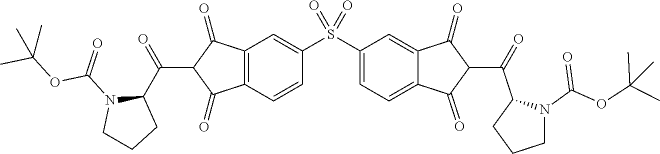 [SMILES: CC(C)(C)OC(=O)N1CCC[C@@H]1C(=O)C1C(=O)c2ccc(S(=O)(=O)c3ccc4c(c3)C(=O)C(C(=O)[C@H]3CCCN3C(=O)OC(C)(C)C)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and N-Boc-D-proline (CAS number 37784-17-1, 323 mg, 1.5 mmol) as a dark colored sodium salt (199 mg, 53%). LC/MS (ESI+): R_{f}=6.57 min, (M+H+Na)⁺=771.84; HRMS (ESI−): calculated for C₃₈H₄₀N₂O₁₂S m/z [M−H]⁻: 747.2229, observed 747.2228; ¹H NMR δ 8.06 (d, J=8.20 Hz, 2H), 7.73 (s, 2H), 7.53 (d, J=8.20 Hz, 2H), 5.17-5.48 (m, 2H), 3.86-4.18 (m, 4H), 1.48-1.81 (m, 4H), 1.16 (s, 6H), 1.35 (s, 12H).

### Example 163—Synthesis of tert-butyl N-(3-{5-[2-(3-{[(tert-butoxy)carbonyl]amino}propanoyl)-1,3-dioxo-2,3-dihydro-1H-indene-5-carbonyl]-1,3-dioxo-2,3-dihydro-1H-inden-2-yl}-3-oxopropyl)carbamate

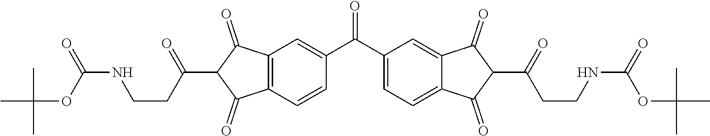 [SMILES: CC(C)(C)OC(=O)NCCC(=O)C1C(=O)c2ccc(C(=O)c3ccc4c(c3)C(=O)C(C(=O)CCNC(=O)OC(C)(C)C)C4=O)cc2C1=O]

In a similar manner to that of Example 111, the title compound was prepared from 5,5′-carbonylbis(1H-indene-1,3(2H)-dione) (Example 4,160.0 mg, 0.5 mmol) and N-Boc-beta-alanine (CAS number 3303-84-2, 284 mg, 1.5 mmol) as a dark colored sodium salt (190 mg, 58%). LC/MS (ESI+): R_{f}=6.02 min, (M+H+Na)⁺=683.77; HRMS (ESI−): calculated for C₃₅H₃₆N₂O₁₁ m/z [M−H]⁻: 659.2246, observed 659.2250; ¹H NMR δ 7.83 (d, J=8.80 Hz, 2H), 7.61 (s, 2H), 7.51 (d, J=7.00 Hz, 2H), 6.40-6.59 (m, 1H), 3.08-3.18 (m, 4H), 2.85-2.99 (m, 6H), 1.35 (br. s, 18H).

### Example 164—Synthesis of tert-butyl N-[3-(5-{[2-(3-{[(tert-butoxy)carbonyl]amino}propanoyl)-1,3-dioxo-2,3-dihydro-1H-inden-5-yl]sulfonyl}-1,3-dioxo-2,3-dihydro-1H-inden-2-yl)-3-oxopropyl]carbamate

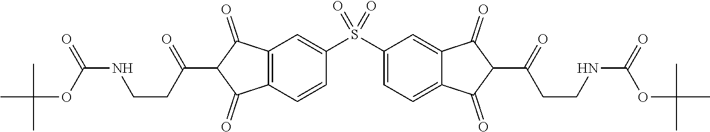 [SMILES: CC(C)(C)OC(=O)NCCC(=O)C1C(=O)c2ccc(S(=O)(=O)c3ccc4c(c3)C(=O)C(C(=O)CCNC(=O)OC(C)(C)C)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and N-Boc-beta-alanine (CAS number 3303-84-2, 284 mg, 1.5 mmol) as a dark colored sodium salt (295 mg, 85%). LC/MS (ESI+): R_{f}=5.83 min, (M+H)⁺=697.51; HRMS (ESI−): calculated for C₃₄H₃₆N₂O₁₂S m/z [M−H]⁻: 695.1916, observed 695.1915; ¹H NMR δ 8.08 (d, J=7.00 Hz, 2H), 7.93 (s, 2H), 7.74 (s, 2H), 7.55 (d, J=8.20 Hz, 2H), 6.38-6.58 (m, 2H), 3.04-3.20 (m, 4H), 1.32 (s, 18H).

### Example 165—Synthesis of benzyl N—[(S)-1-(5-{2-[(2S)-2-{[(benzyloxy)carbonyl]amino}butanoyl]-1,3-dioxo-2,3-dihydro-1H-indene-5-carbonyl}-1,3-dioxo-2,3-dihydro-1H-inden-2-yl)-1-oxobutan-2-yl]carbamate

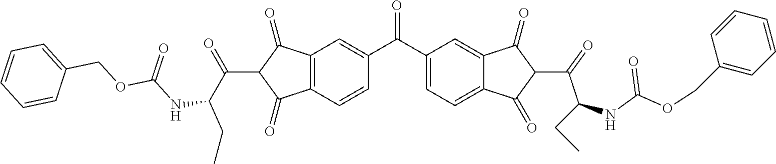 [SMILES: CC[C@H](NC(=O)OCc1ccccc1)C(=O)C1C(=O)c2ccc(C(=O)c3ccc4c(c3)C(=O)C(C(=O)[C@H](CC)NC(=O)OCc3ccccc3)C4=O)cc2C1=O]

In a similar manner to that of Example 111, the title compound was prepared from 5,5′-carbonylbis(1H-indene-1,3(2H)-dione) (Example 4, 160.0 mg, 0.5 mmol) and N-benzyloxycarbonyl-L-2-aminobutyric acid (CAS number 42918-86-5, 356 mg, 1.5 mmol) as a dark colored sodium salt (131 mg, 35%). LC/MS (ESI+): R_{f}=6.58 min, (M+H)⁺=757.56; HRMS (ESI−): calculated for C₄₃H₃₆N₂O₁₁ m/z [M−H]⁻: 755.2246, observed 755.2243; ¹H NMR δ 7.82-8.03 (m, 2H), 7.48-7.80 (m, 4H), 7.09-7.48 (m, 10H), 5.05 (d, J=34.60 Hz, 4H), 1.24-1.87 (m, 4H), 0.86 (br. s, 6H).

### Example 166—Synthesis of benzyl N-[(2S)-1-[5-({2-[(2S)-2-{[(benzyloxy)carbonyl]amino}-1-hydroxybutyl]-1,3-dioxo-2,3-dihydro-1H-inden-5-yl}sulfonyl)-1,3-dioxo-2,3-dihydro-1H-inden-2-yl]-1-oxobutan-2-yl]carbamate

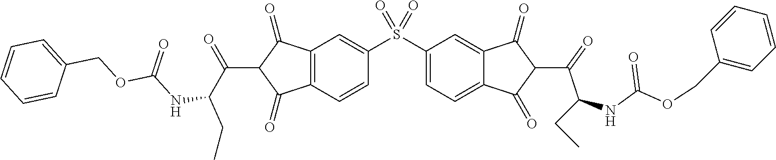 [SMILES: CC[C@H](NC(=O)OCc1ccccc1)C(=O)C1C(=O)c2ccc(S(=O)(=O)c3ccc4c(c3)C(=O)C(C(=O)[C@H](CC)NC(=O)OCc3ccccc3)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and N-benzyloxycarbonyl-L-2-aminobutyric acid (CAS number 42918-86-5, 356 mg, 1.5 mmol) as a dark colored sodium salt (12 mg, 3%). LC/MS (ESI+): R_{f}=6.40 min, (M+H)⁺=793.60; HRMS (ESI−): calculated for C₄₂H₃₆N₂O₁₂S m/z [M−H]⁻: 791.1916, observed 791.1913; ¹H NMR δ 8.09 (d, J=7.00 Hz, 2H), 7.76 (s, 2H), 7.52 (d, J=7.60 Hz, 2H), 7.32 (br. s., 10H), 4.85-5.02 (m, 4H), 1.21-1.82 (m, 4H), 0.81 (s, 6H).

### Example 167—Synthesis of 2-[(2S)-1-methylpyrrolidine-2-carbonyl]-5-({2-[(2S)-1-methylpyrrolidine-2-carbonyl]-1,3-dioxo-2,3-dihydro-1H-inden-5-yl}sulfonyl)-2,3-dihydro-1H-indene-1,3-dione

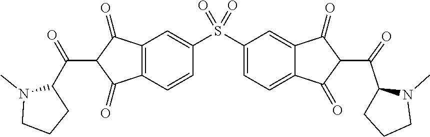

In a similar manner to that of Example 136, except that the sodium salt was not prepared, the title compound was prepared from 5,5′-sulfonylbis(1H-indene-1,3(2H)-dione) (Example 12, 177 mg, 0.5 mmol) and N-methyl-L-proline (CAS number 475-11-6, 194 mg, 1.5 mmol). The product was purified with a C18 reverse phase column using acetonitrile/water and 0.1% ammonium hydroxide modifier. The resulting fractions were concentrated as the title compound (59 mg, 20%). LC/MS (ESI+): R_{f}=3.04 min, (M+H)⁺=577.55; HRMS (ESI−): calculated for C₃₀H₂₈N₂O₈S m/z [M−H]⁻: 575.1526, observed 575.1494; ¹H NMR δ 8.15 (d, J=8.20 Hz, 2H), 7.83 (s, 2H), 7.63 (d, J=7.60 Hz, 2H), 4.69-4.95 (m, 2H), 3.46-3.59 (m, 2H), 3.04-3.17 (m, 2H), 2.71 (s, 6H), 2.53-2.62 (m, 2H), 1.87-2.07 (m, 2H), 1.50-1.81 (m, 4H).

### Example 168—Synthesis of 5-{1,3-dioxo-2-[2-(1H-1,2,3,4-tetrazol-1-yl)acetyl]-2,3-dihydro-1H-indene-5-carbonyl}-2-[2-(1H-1,2,3,4-tetrazol-1-yl)acetyl]-2,3-dihydro-1H-indene-1,3-dione

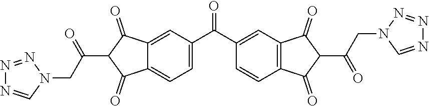 [SMILES: O=C(c1ccc2c(c1)C(=O)C(C(=O)Cn1cnnn1)C2=O)c1ccc2c(c1)C(=O)C(C(=O)Cn1cnnn1)C2=O]

In a similar manner to that of Example 111, except the sodium salt was not prepared, the title compound was prepared from 5,5′-carbonylbis(1H-indene-1,3(2H)-dione) (Example 4, 160.0 mg, 0.5 mmol) and 1-H-1,2,3,4-tetrazole-1-acetic acid (CAS number 21732-17-2, 192 mg, 1.5 mmol). The product was purified with a C18 reverse phase column using acetonitrile/water and 0.1% ammonium hydroxide modifier. The resulting fractions were concentrated as the title compound (18 mg, 7%). LC/MS (ESI+): R_{f}=3.54 min, (M+H)⁺=539.40; HRMS (ESI−): calculated for C₂₅H₁₄N₈O₇ m/z [M−H]⁻: 537.0892, observed 537.0913; ¹H NMR δ 9.23 (s, 2H), 7.88 (d, J=7.60 Hz, 2H), 7.70 (s, 2H), 7.59 (d, J=7.00 Hz, 2H), 5.72 (s, 4H).

### Example 169—Synthesis of 5-({1,3-dioxo-2-[2-(1H-1,2,3,4-tetrazol-1-yl)acetyl]-2,3-dihydro-1H-inden-5-yl}sulfonyl)-2-[2-(1H-1,2,3,4-tetrazol-1-yl)acetyl]-2,3-dihydro-1H-indene-1,3-dione

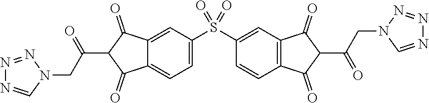 [SMILES: O=C(Cn1cnnn1)C1C(=O)c2ccc(S(=O)(=O)c3ccc4c(c3)C(=O)C(C(=O)Cn3cnnn3)C4=O)cc2C1=O]

In a similar manner to that of Example 136, except that the sodium salt was not prepared, the title compound was prepared from 5,5′-sulfonylbis(1H-indene-1,3(2H)-dione) (Example 12,177 mg, 0.5 mmol) and 1-H-1,2,3,4-tetrazole-1-acetic acid (CAS number 21732-17-2, 192 mg, 1.5 mmol). The product was purified with a C18 reverse phase column using acetonitrile/water and 0.1% ammonium hydroxide modifier. The resulting fractions were concentrated as the title compound (19 mg, 7%). LC/MS (ESI+): R_{f}=3.73 min, (M+H)⁺=575.44; HRMS (ESI−): calculated for C₂₄H₁₄N₈O₈S m/z [M−H]⁻: 573.0583, observed 573.0589; ¹H NMR δ 9.23 (s, 2H), 8.16 (d, J=7.60 Hz, 2H), 7.86 (s, 2H), 7.64 (d, J=7.03 Hz, 2H), 5.70 (s, 4H).

### Example 170—Synthesis of tert-butyl N-[(1R)-2-(5-{2-[(2R)-2-{[(tert-butoxy)carbonyl]amino}-2-phenylacetyl]-1,3-dioxo-2,3-dihydro-1H-indene-5-carbonyl}-1,3-dioxo-2,3-dihydro-1H-inden-2-yl)-2-oxo-1-phenylethyl]carbamate

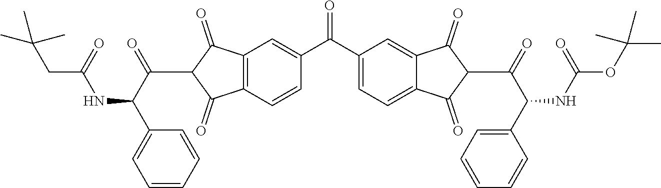 [SMILES: CC(C)(C)CC(=O)N[C@@H](C(=O)C1C(=O)c2ccc(C(=O)c3ccc4c(c3)C(=O)C(C(=O)[C@H](NC(=O)OC(C)(C)C)c3ccccc3)C4=O)cc2C1=O)c1ccccc1]

In a similar manner to that of Example 111, the title compound was prepared from 5,5′-carbonylbis(1H-indene-1,3(2H)-dione) (Example 4, 160.0 mg, 0.5 mmol) and N-Boc-D-phenylglycine (CAS number 33125-05-2, 377 mg, 1.5 mmol) as a dark colored sodium salt (152 mg, 39%). LC/MS (ESI+): R_{f}=7.49 min, (M+H−(Boc)₂)⁺=585.44; HRMS (ESI−): calculated for C₄₅H₄₀N₂O₁₁ m/z [M−H]⁻: 783.2559, observed 783.2572; ¹H NMR δ 7.78-7.85 (m, 2H), 7.61 (s, 2H), 7.48-7.57 (m, 2H), 7.39 (d, J=6.40 Hz, 4H), 7.20 (t, J=7.00 Hz, 4H), 7.06-7.16 (m, 2H), 6.55-6.69 (m, 2H), 6.45-6.54 (m, 2H), 1.34 (s, 18H).

### Example 171—Synthesis of 2-(5-methylhexanoyl)-5-{[2-(5-methylhexanoyl)-1,3-dioxo-2,3-dihydro-1H-inden-5-yl]sulfonyl}-2,3-dihydro-1H-indene-1,3-dione

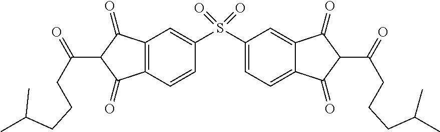 [SMILES: CC(C)CCCC(=O)C1C(=O)c2ccc(S(=O)(=O)c3ccc4c(c3)C(=O)C(C(=O)CCCC(C)C)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and 5-methylhexanoic acid (CAS number 628-46-6, 215 mg, 1.5 mmol) as a dark colored sodium salt (41 mg, 14%). LC/MS (ESI+): R_{f}=8.19 min, (M+H+ACN)⁺=620.60; HRMS (ESI−): calculated for C₃₂H₃₄O₈S m/z [M−H]⁻: 577.1902, observed 577.1921; ¹H NMR δ 8.05 (d, J=7.60 Hz, 2H), 7.71 (s, J=4.04 Hz, 2H), 7.52 (d, J=7.60 Hz, 2H), 2.64 (t, J=7.62 Hz, 4H), 1.38-1.53 (m, 6H), 1.08-1.17 (m, 4H), 0.82 (d, J=6.40 Hz, 12H).

### Example 172—Synthesis of 2-[(2R)-2-amino-2-phenylacetyl]-5-({2-[(2R)-2-amino-2-phenylacetyl]-1,3-dioxo-2,3-dihydro-1H-inden-5-yl}sulfonyl)-2,3-dihydro-1H-indene-1,3-dione

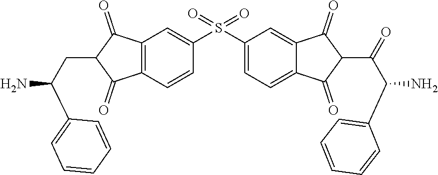 [SMILES: N[C@@H](CC1C(=O)c2ccc(S(=O)(=O)c3ccc4c(c3)C(=O)C(C(=O)[C@H](N)c3ccccc3)C4=O)cc2C1=O)c1ccccc1]

In a similar manner to that of Example 136, the title compound was prepared from 5,5′-sulfonylbis(1H-indene-1,3(2H)-dione) (Example 12, 177 mg, 0.5 mmol) and N-Boc-D-phenylglycine (CAS number 33125-05-2, 251 mg, 1.5 mmol). The resulting precipitant treated with 1% Acetic Acid in 1:5 methanol:methylene chloride and filtered to provide the title compound (2 mg, 1%). LC/MS (ESI+): R_{f}=3.33 min, (M+H)⁺=621.35; HRMS (ESI−): calculated for C₃₄H₂₄N₂O₈S m/z [M−H]⁻: 619.1191, observed 619.1191; ¹H NMR δ 8.24-8.43 (m, 4H), 8.03-8.19 (m, 2H), 7.80 (s, 2H), 7.54-7.72 (m, 2H), 7.39-7.54 (m, 4H), 7.15-7.39 (m, 4H), 5.93-6.18 (m, 2H).

### Example 173—Synthesis of tert-butyl N-(2-{5-[2-(2-{[(tert-butoxy)carbonyl]amino}acetyl)-1,3-dioxo-2,3-dihydro-1H-indene-5-carbonyl]-1,3-dioxo-2,3-dihydro-1H-inden-2-yl}-2-oxoethyl)carbamate

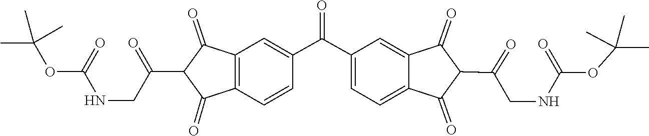 [SMILES: CC(C)(C)OC(=O)NCC(=O)C1C(=O)c2ccc(C(=O)c3ccc4c(c3)C(=O)C(C(=O)CNC(=O)OC(C)(C)C)C4=O)cc2C1=O]

In a similar manner to that of Example 111, the title compound was prepared from 5,5′-carbonylbis(1H-indene-1,3(2H)-dione) (Example 4, 160.0 mg, 0.5 mmol) and 2-(tert-butoxy)-carbonylamino acetic acid (CAS number 4530-20-5, 263 mg, 1.5 mmol) as a dark colored sodium salt (142 mg, 45%). LC/MS (ESI+): R_{f}=5.75 min, (M+H+Na)⁺=655.76; HRMS (ESI−): calculated for C₃₃H₃₂N₂O₁₁ m/z [M−H]⁻: 631.1933, observed 631.1939; ¹H NMR δ 7.83 (d, J=7.62 Hz, 2H), 7.62 (br. s., 2H), 7.52 (d, J=7.03 Hz, 2H), 4.13 (d, J=5.27 Hz, 4H), 1.38 (s, 18H).

### Example 174—Synthesis of tert-butyl N-[2-(5-{[2-(2-{[(tert-butoxy)carbonyl]amino}acetyl)-1,3-dioxo-2,3-dihydro-1H-inden-5-yl]sulfonyl}-1,3-dioxo-2,3-dihydro-1H-inden-2-yl)-2-oxoethyl]carbamate

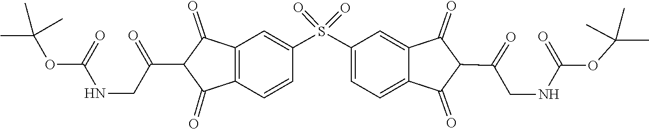 [SMILES: CC(C)(C)OC(=O)NCC(=O)C1C(=O)c2ccc(S(=O)(=O)c3ccc4c(c3)C(=O)C(C(=O)CNC(=O)OC(C)(C)C)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and 2-(tert-butoxy)-carbonylamino acetic acid (CAS number 4530-20-5, 263 mg, 1.5 mmol) as a dark colored sodium salt (250 mg, 75%). LC/MS (ESI+): R_{f}=5.60 min, (M+H−(Boc)₂)⁺=469.22; HRMS (ESI−): calculated for C₃₂H₃₂N₂O₁₂S m/z [M−H]⁻: 667.1603, observed 667.1628; ¹H NMR δ 8.09 (dd, J=1.76, 7.62 Hz, 2H), 7.76 (d, J=1.17 Hz, 2H), 7.56 (d, J=7.62 Hz, 2H), 4.10 (d, J=5.27 Hz, 4H), 1.36 (br. s, 18H).

### Example 175—Synthesis of N-[2-(5-{[2-(2-acetamidoacetyl)-1,3-dioxo-2,3-dihydro-1H-inden-5-yl]sulfonyl}-1,3-dioxo-2,3-dihydro-1H-inden-2-yl)-2-oxoethyl]acetamide

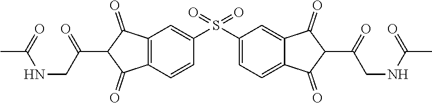 [SMILES: CC(=O)NCC(=O)C1C(=O)c2ccc(S(=O)(=O)c3ccc4c(c3)C(=O)C(C(=O)CNC(C)=O)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and N-acetyl glycine (CAS number 543-24-8, 263 mg, 1.5 mmol) as a dark colored sodium salt (22 mg, 8%). LC/MS (ESI+): R_{f}=2.94 min, purity=78%, (M+H)⁺=553.41; HRMS (ESI−): calculated for C₂₆H₂₀N₂O₁₀S m/z [M−H]⁻: 551.0766, observed 551.0765; ¹H NMR δ 8.12 (dd, J=1.46, 6.74 Hz, 2H), 7.81 (s, 2H), 7.60 (d, J=7.03 Hz, 2H), 4.26 (br. s., 4H), 1.87 (s, 6H).

### Example 176—Synthesis of 2-(3-methoxypropanoyl)-5-[2-(3-methoxypropanoyl)-1,3-dioxo-2,3-dihydro-1H-indene-5-carbonyl]-2,3-dihydro-1H-indene-1,3-dione

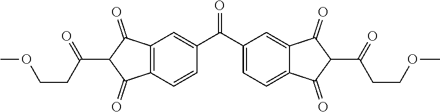

In a similar manner to that of Example 111, except the sodium salt was not prepared, the title compound was prepared from 5,5′-carbonylbis(1H-indene-1,3(2H)-dione) (Example 4, 160.0 mg, 0.5 mmol) and 3-methoxypropionic acid (CAS number 374547, 141 μL, 1.5 mmol). The product was further purified using column chromatography with methylene chloride/20% MeOH in methylene chloride with a 1% acetic acid modifier. The resulting fractions were concentrated, washed with 1 N HCl, and filtered to afford the title compound (9 mg, 4%). LC/MS (ESI+): R_{f}=5.84 min, (M+H)⁺=491.59; HRMS (ESI−): calculated for C₂₇H₂₂O₉ m/z [M−H]⁻: 489.1191, observed 489.1190; ¹H NMR (pyridine-d₅) δ 8.43 (s, 2H), 8.11 (dd, J=1.17, 7.62 Hz, 2H), 7.96 (d, J=7.62 Hz, 2H), 4.10 (t, J=5.90 Hz, 4H), 3.84 (t, J=7.60 Hz, 4H), 3.37 (s, 6H).

### Example 177—Synthesis of 2-(3-methoxypropanoyl)-5-{[2-(3-methoxypropanoyl)-1,3-dioxo-2,3-dihydro-1H-inden-5-yl]sulfonyl}-2,3-dihydro-1H-indene-1,3-dione

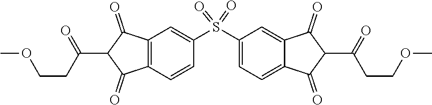

In a similar manner to that of Example 136, except that the sodium salt was not prepared, the title compound was prepared from 5,5′-sulfonylbis(1H-indene-1,3(2H)-dione) (Example 12, 177 mg, 0.5 mmol) and 3-methoxypropionic acid (CAS number 374547, 141 μL, 1.5 mmol). The product was further purified using column chromatography with methylene chloride/20% MeOH in methylene chloride with a 1% acetic acid modifier. The resulting fractions were concentrated, washed with 1 N HCl, and filtered to afford the title compound (2 mg, 1%). LC/MS (ESI+): R_{f}=5.11 min, (M+H)⁺=527.63; HRMS (ESI−): calculated for C₂₆H₂₂O₁₀S m/z [M−H]⁻: 525.0861, observed 525.0849; ¹H NMR (pyridine-d₅) δ 8.21 (d, J=8.40 Hz, 2H), 7.99 (s, 2H), 7.83 (d, J=7.60 Hz, 2H), 3.98 (t, J=6.74 Hz, 4H), 3.68-3.79 (m, 4H), 3.30 (s, 3H), 3.17 (s, 3H).

### Example 178—Synthesis of 2-[3-(2-methoxyethoxy)propanoyl]-5-({2-[3-(2-methoxyethoxy)propanoyl]-1,3-dioxo-2,3-dihydro-1H-inden-5-yl}sulfonyl)-2,3-dihydro-1H-indene-1,3-dione

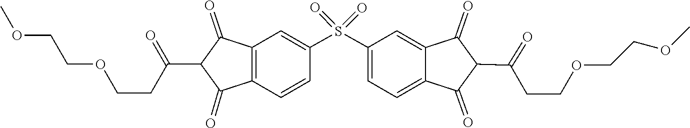

In a similar manner to that of Example 136, except that the sodium salt was not prepared, the title compound was prepared from 5,5′-sulfonylbis(1H-indene-1,3(2H)-dione) (Example 12, 177 mg, 0.5 mmol) and 3-(2-methoxyethoxy) propanoic acid (CAS number 149577-05-9, 222 mg, 1.5 mmol). The product was purified using column chromatography with a gradient up to 20% MeOH in Methylene Chloride with a 1% acetic acid modifier. The corresponding fractions were concentrated, washed with 1 N HCl, and filtered to afford the title compound (87 mg, 28%). LC/MS (ESI+): R_{f}=4.78 min, (M+H)⁺=615.70; HRMS (ESI−): calculated for C₃₀H₃₀O₁₂S m/z [M−H]⁻: 613.1385, observed 613.1380; ¹H NMR (pyridine-d₅) δ 8.63 (s, 2H), 8.39 (dd, J=1.17, 7.62 Hz, 2H), 7.91 (d, J=7.62 Hz, 2H), 4.14 (t, J=6.40 Hz, 2H), 3.92-4.03 (m, 2H), 3.67-3.81 (m, 6H), 3.63 (t, J=5.30 Hz, 2H), 3.51 (dd, J=3.81, 6.15 Hz, 4H), 3.22 (s, 6H).

### Example 179—Synthesis of tert-butyl N-[(2S)-1-(5-{2-[(2S)-2-{[(tert-butoxy)carbonyl]amino}propanoyl]-1,3-dioxo-2,3-dihydro-1H-indene-5-carbonyl}-1,3-dioxo-2,3-dihydro-1H-inden-2-yl)-1-oxopropan-2-yl]carbamate

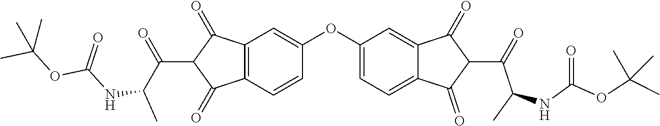 [SMILES: C[C@H](NC(=O)OC(C)(C)C)C(=O)C1C(=O)c2ccc(Oc3ccc4c(c3)C(=O)C(C(=O)[C@H](C)NC(=O)OC(C)(C)C)C4=O)cc2C1=O]

In a similar manner to that of Example 111, except the sodium salt was not prepared, the title compound was prepared from 5,5′-carbonylbis(1H-indene-1,3(2H)-dione) (Example 4, 160.0 mg, 0.5 mmol) and N-Boc-L-alanine (CAS number 15761-38-3, 284 mg, 1.5 mmol) to afford the title compound (225 mg, 68%). LC/MS (ESI+): R_{f}=6.72 min, (M−2Boc+H)⁺=461.40; HRMS (ESI−): calculated for C₃₅H₃₅N₂O₁₁ m/z [M−H]⁻: 659.2246, observed 659.2246; ¹H NMR δ 7.99 (d, J=8.0 Hz, 2H), 7.82 (s, 2H), 7.75 (d, J=8.80 Hz, 2H), 5.11-5.25 (m, 2H), 1.36 (br. s, 18H), 1.21 (d, J=7.03 Hz, 6H).

### Example 180—Synthesis of tert-butyl N-[(2S)-1-[5-({2-[(2S)-2-{[(tert-butoxy)carbonyl]amino}propanoyl]-1,3-dioxo-2,3-dihydro-1H-inden-5-yl}sulfonyl)-1,3-dioxo-2,3-dihydro-1H-inden-2-yl]-1-oxopropan-2-yl]carbamate

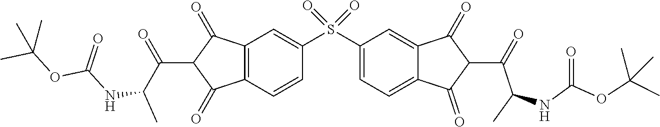 [SMILES: C[C@H](NC(=O)OC(C)(C)C)C(=O)C1C(=O)c2ccc(S(=O)(=O)c3ccc4c(c3)C(=O)C(C(=O)[C@H](C)NC(=O)OC(C)(C)C)C4=O)cc2C1=O]

In a similar manner to that of Example 136, except that the sodium salt was not prepared, the title compound was prepared from 5,5′-sulfonylbis(1H-indene-1,3(2H)-dione) (Example 12,177 mg, 0.5 mmol) and N-Boc-L-alanine (CAS number 15761-38-3, 284 mg, 1.5 mmol) to afford the title compound (264 mg, 76%). LC/MS (ESI+): R_{f}=6.63 min, (M-2Boc+H)⁺=497.37; HRMS (ESI−): calculated for C₃₄H₃₅N₂O₁₂S m/z [M−H]⁻: 695.1916, observed 695.1889; ¹H NMR δ 8.13 (dd, J=1.76, 7.62 Hz, 2H), 7.83 (s, 2H), 7.61 (d, J=7.00 Hz, 2H), 4.95-5.17 (m, 2H), 1.34 (s, 18H), 1.10 (d, J=7.00 Hz, 6H).

### Example 181—Synthesis of N-(2-{5-[2-(2-acetamidoacetyl)-1,3-dioxo-2,3-dihydro-1H-indene-5-carbonyl]-1,3-dioxo-2,3-dihydro-1H-inden-2-yl}-2-oxoethyl)acetamide

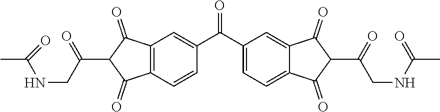 [SMILES: CC(=O)NCC(=O)C1C(=O)c2ccc(C(=O)c3ccc4c(c3)C(=O)C(C(=O)CNC(C)=O)C4=O)cc2C1=O]

In a similar manner to that of Example 111, except the sodium salt was not prepared, the title compound was prepared from 5,5′-carbonylbis(1H-indene-1,3(2H)-dione) (Example 4, 160.0 mg, 0.5 mmol) and N-acetyl glycine (CAS number 543-24-8, 176 mg, 1.5 mmol) to afford the title compound (170 mg, 66%). LC/MS (ESI+): R_{f}=3.11 min, (M+H)⁺=517.50; HRMS (ESI−): calculated for C₂₇H₁₉N₂O₉ m/z [M−H]⁻: 515.1096, observed 515.1064; ¹H NMR δ 7.87-8.01 (m, 2H), 7.76 (s, 2H), 7.67 (d, J=7.62 Hz, 2H), 4.37 (s, 4H), 1.88 (s, 6H).

### Example 182—Synthesis of tert-butyl N-[(1R)-2-[5-({2-[(2R)-2-{[(tert-butoxy)carbonyl]amino}-1-hydroxy-2-phenylethyl]-1,3-dioxo-2,3-dihydro-1H-inden-5-yl}sulfonyl)-1,3-dioxo-2,3-dihydro-1H-inden-2-yl]-2-oxo-1-phenylethyl]carbamate

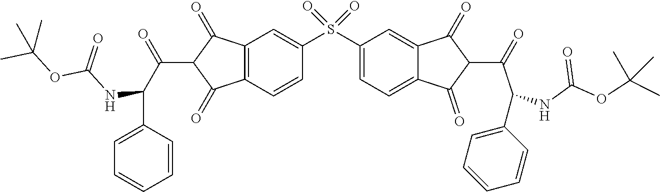 [SMILES: CC(C)(C)OC(=O)N[C@@H](C(=O)C1C(=O)c2ccc(S(=O)(=O)c3ccc4c(c3)C(=O)C(C(=O)[C@H](NC(=O)OC(C)(C)C)c3ccccc3)C4=O)cc2C1=O)c1ccccc1]

In a similar manner to that of Example 136, except that the sodium salt was not prepared, the title compound was prepared from 5,5′-sulfonylbis(1H-indene-1,3(2H)-dione) (Example 12, 177 mg, 0.5 mmol) and N-Boc-D-phenylglycine (CAS number 33125-05-2, 377 mg, 1.5 mmol) to afford the title compound (217 mg, 53%). LC/MS (ESI+): R_{f}=8.90 min, (M-2Boc+H)⁺=621.55; HRMS (ESI−): calculated for C₄₄H₃₉N₂O₁₂ m/z [M−H]⁻: 819.2229, observed 819.2237; ¹H NMR δ 8.06 (dd, J=1.76, 7.62 Hz, 2H), 7.77 (s, 2H), 7.54 (d, J=7.62 Hz, 2H), 7.34 (d, J=7.03 Hz, 4H), 7.17 (t, J=7.60 Hz, 4H), 7.11 (d, J=7.60 Hz, 2H), 6.40-6.50 (m, 2H), 1.34 (br. s, 18H).

### Example 183—Synthesis of 5-{1,3-dioxo-2-[2-(2-oxopyrrolidin-1-yl)acetyl]-2,3-dihydro-1H-indene-5-carbonyl}-2-[2-(2-oxopyrrolidin-1-yl)acetyl]-2,3-dihydro-1H-indene-1,3-dione

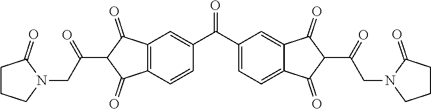 [SMILES: O=C(c1ccc2c(c1)C(=O)C(C(=O)CN1CCCC1=O)C2=O)c1ccc2c(c1)C(=O)C(C(=O)CN1CCCC1=O)C2=O]

In a similar manner to that of Example 111, except the sodium salt was not prepared, the title compound was prepared from 5,5′-carbonylbis(1H-indene-1,3(2H)-dione) (Example 4, 160.0 mg, 0.5 mmol) and (2-oxo-pyrrolidin-1-yl) acetic acid (CAS number 53934-76-2, 215 mg, 1.5 mmol) to afford the title compound (82 mg, 29%). LC/MS (ESI+): R_{f}=3.17 min, (M+H)⁺=569.80; HRMS (ESI−): calculated for C₃₁H₂₃N₂O₉ m/z [M−H]⁻: 567.1409, observed 567.1428; ¹H NMR δ 7.86 (d, J=7.62 Hz, 2H), 7.64 (s, 2H), 7.54 (d, J=7.03 Hz, 2H), 4.38 (s, 4H), 3.23-3.28 (m, 2H), 2.23 (t, J=7.91 Hz, 4H), 1.93 (quin, J=7.30 Hz, 4H).

### Example 184—Synthesis of 5-({1,3-dioxo-2-[2-(2-oxopyrrolidin-1-yl)acetyl]-2,3-dihydro-1H-inden-5-yl}sulfonyl)-2-[2-(2-oxopyrrolidin-1-yl)acetyl]-2,3-dihydro-1H-indene-1,3-dione

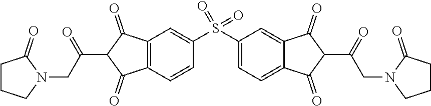 [SMILES: O=C(CN1CCCC1=O)C1C(=O)c2ccc(S(=O)(=O)c3ccc4c(c3)C(=O)C(C(=O)CN3CCCC3=O)C4=O)cc2C1=O]

In a similar manner to that of Example 136, except that the sodium salt was not prepared, the title compound was prepared from 5,5′-sulfonylbis(1H-indene-1,3(2H)-dione) (Example 12, 177 mg, 0.5 mmol) and (2-oxo-pyrrolidin-1-yl) acetic acid (CAS number 53934-76-2, 215 mg, 1.5 mmol) to afford the title compound (62 mg, 19%). LC/MS (ESI+): R_{f}=3.17 min, (M+H)⁺=605.77; HRMS (ESI−): calculated for C₃₀H₂₃N₂O₁₀S m/z [M−H]⁻: 603.1079, observed 603.1088; ¹H NMR δ 8.11 (dd, J=1.76, 7.62 Hz, 2H), 7.79 (s, 2H), 7.59 (d, J=7.62 Hz, 2H), 4.35 (s, 4H), 2.21 (t, J=8.80 Hz, 4H), 1.83-1.98 (m, 4H).

### Example 185—Synthesis of N-[2-(5-{2-[2-(3,3-dimethylbutanamido)acetyl]-1,3-dioxo-2,3-dihydro-1H-indene-5-carbonyl}-1,3-dioxo-2,3-dihydro-1H-inden-2-yl)-2-oxoethyl]-3,3-dimethylbutanamide

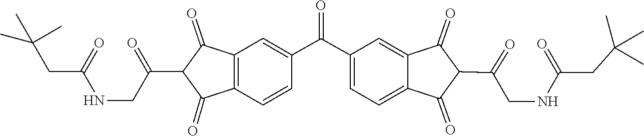 [SMILES: CC(C)(C)CC(=O)NCC(=O)C1C(=O)c2ccc(C(=O)c3ccc4c(c3)C(=O)C(C(=O)CNC(=O)CC(C)(C)C)C4=O)cc2C1=O]

3,3-Dimethylbutyryl chloride (CAS number 7065-46-5, 556 μL, 4 mmol) was added dropwise to a solution containing glycine (300 mg, 4 mmol) and Na₂CO₃ (1187 mg, 11.2 mmol) in 2:1 water:1,4-dioxane (30 ml) and the mixture was stirred at rt overnight. The solution was acidified with 2N HCl and extracted with ethyl acetate, concentrated, and dried in vacuo to obtain 2-(3,3-dimethylbutanamido) acetic acid (CAS number 926-04-5, 280 mg, 40%). ¹H NMR (CD₃OD) δ 3.88 (s, 2H), 2.14 (d, J=12.30 Hz, 2H), 1.04 (br. s, 9H).

In a similar manner to that of Example 111, the title compound was prepared from 5,5′-carbonylis(1H-indene-1,3(2H)-dione) (Example 4, 160.0 mg, 0.5 mmol) and 2-(3,3-dimethylbutanamido) acetic acid (CAS number 926-04-5, 260 mg, 1.5 mmol). The bis sodium salt was dissolved in MeOH:acetonitrile 80:20 and the precipitated with 1N HCl and filtered to afford the title compound (92 mg, 29%). LC/MS (ESI+): R_{f}=5.23 min, (M+H)⁺=629.64; HRMS (ESI−): calculated for C₃₅H₃₅N₂O₉ m/z [M−H]⁻: 627.2348, observed 627.2337; ¹H NMR δ 7.92 (dd, J=1.76, 7.62 Hz, 2H), 7.74 (s, 2H), 7.65 (d, J=7.62 Hz, 2H), 4.34 (br. s, 4H), 2.04 (s, 4H), 0.97 (s, 18H).

### Example 186—Synthesis of N-{2-[5-({2-[2-(3,3-dimethylbutanamido)acetyl]-1,3-dioxo-2,3-dihydro-1H-inden-5-yl}sulfonyl)-1,3-dioxo-2,3-dihydro-1H-inden-2-yl]-2-oxoethyl}-3,3-dimethylbutanamide

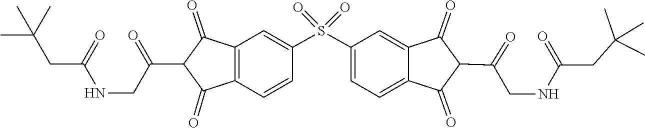 [SMILES: CC(C)(C)CC(=O)NCC(=O)C1C(=O)c2ccc(S(=O)(=O)c3ccc4c(c3)C(=O)C(C(=O)CNC(=O)CC(C)(C)C)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and 2-(3,3-dimethylbutanamido) acetic acid (prepared in Example 185, CAS number 926-04-5, 260 mg, 1.5 mmol). The bis sodium salt was dissolved in MeOH:acetonitrile 80:20 and the precipitated with 1N HCl and filtered to afford the title compound (89 mg, 27%). LC/MS (ESI+): R_{f}=4.63 min, (M+H)⁺=665.96; HRMS (ESI−): calculated for C₃₄H₃₅N₂O₁₀S m/z [M−H]⁻: 663.2018, observed 663.2013; ¹H NMR δ 8.09 (dd, J=1.80, 7.60 Hz, 2H), 7.78 (s, 2H), 7.56 (d, J=7.62 Hz, 2H), 4.23 (br. s., 4H), 2.01 (s, 4H), 0.95 (s, 18H).

### Example 187—Synthesis of 2-methyl-N-[2-(5-{2-[2-(2-methylpropanamido)acetyl]-1,3-dioxo-2,3-dihydro-1H-indene-5-carbonyl}-1,3-dioxo-2,3-dihydro-1H-inden-2-yl)-2-oxoethyl]propanamide

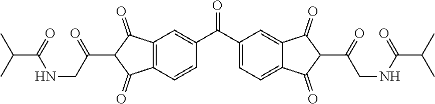 [SMILES: CC(C)C(=O)NCC(=O)C1C(=O)c2ccc(C(=O)c3ccc4c(c3)C(=O)C(C(=O)CNC(=O)C(C)C)C4=O)cc2C1=O]

Isobutyryl chloride (CAS number: 79-30-1, 419 μL, 4 mmol) was added dropwise to a solution containing glycine (300 mg, 4 mmol) and Na₂CO₃ (1187 mg, 11.2 mmol) in 2:1 water:1,4-dioxane (30 ml) and the mixture was stirred at rt overnight. The solution was acidified with 2N HCl and extracted with ethyl acetate, concentrated, and dried in vacuo to obtain (isobutyrylamino)acetic acid (CAS number 15926-18-8, 170 mg, 29%). ¹H NMR (CD₃OD) δ 3.87 (s, 2H), 2.50 (quin, J=6.70 Hz, 1H), 1.13 (d, J=7.60 Hz, 6H).

In a similar manner to that of Example 111, the title compound was prepared from 5,5′-carbonylbis(1H-indene-1,3(2H)-dione) (Example 4, 107 mg, 0.33 mmol) and (isobutyrylamino)acetic acid (CAS number 15926-18-8, 150 mg, 1.0 mmol) to afford the title compound as the bis sodium salt (152 mg, 71%). LC/MS (ESI+): R_{f}=4.20 min, (M+H)⁺=573.61; HRMS (ESI−): calculated for C₃₁H₂₇N₂O₉ m/z [M−H]⁻: 571.1722, observed 571.1717; ¹H NMR δ 7.88 (dd, J=1.20, 7.60 Hz, 2H), 7.67 (s, 2H), 7.58 (d, J=7.62 Hz, 2H), 4.29 (d, J=4.69 Hz, 4H), 2.69-2.75 (m, 2H), 1.02 (d, J=7.00 Hz, 12H).

### Example 188—Synthesis of (3S)-3-{[(tert-butoxy)carbonyl]amino}-4-(5-{2-[(2S)-2-{[(tert-butoxy)carbonyl]amino}-3-carboxypropanoyl]-1,3-dioxo-2,3-dihydro-1H-indene-5-carbonyl}-1,3-dioxo-2,3-dihydro-1H-inden-2-yl)-4-oxobutanoic acid

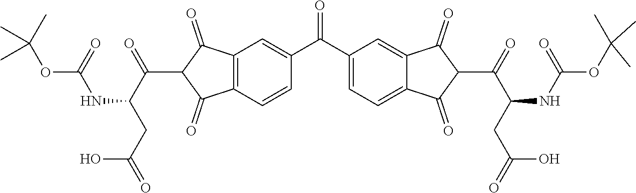 [SMILES: CC(C)(C)OC(=O)N[C@@H](CC(=O)O)C(=O)C1C(=O)c2ccc(C(=O)c3ccc4c(c3)C(=O)C(C(=O)[C@H](CC(=O)O)NC(=O)OC(C)(C)C)C4=O)cc2C1=O]

In a similar manner to that of Example 111, the title compound was prepared from 5,5′-carbonylbis(1H-indene-1,3(2H)-dione) (Example 4, 160.0 mg, 0.5 mmol) and boc-L-aspartic acid 4-methyl ester (CAS number 59768-74-0, 371 mg, 1.5 mmol). To ensure the methyl ester hydrolysis was complete, the filtered solid (330 mg, 0.4 mmol) was dissolved in THF (3 ml)/water (3 ml) and lithium hydroxide (126 mg, 2.8 mmol) was added and the reaction stirred at rt for 10 min. The mixture was neutralized with 1 N HCl and a solid precipitant was filtered. The solid was sonicated in methylene chloride and 2N NaOH. The resulting precipitant was filtered to afford the title compound as the tetra-sodium salt (108 mg, 29%). LC/MS (ESI+): R_{f}=4.60 min, (M−2Boc+H)⁺=549.80; HRMS (ESI−): calculated for C₃₇H₃₅N₂O₁₅ m/z [M−H]⁻: 747.201985, observed 747.204292; ¹H NMR δ 7.93 (dd, J=1.20, 7.20 Hz, 2H), 7.75 (s, 2H), 7.65 (d, J=7.00 Hz, 2H), 5.23-5.32 (m, 2H).

### Example 189—Synthesis of 2-(5-methylhexanoyl)-5-[2-(5-methylhexanoyl)-1,3-dioxo-2,3-dihydro-1H-indene-5-carbonyl]-2,3-dihydro-1H-indene-1,3-dione

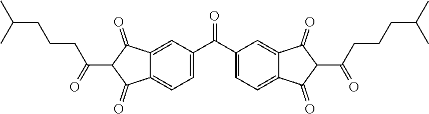 [SMILES: CC(C)CCCC(=O)C1C(=O)c2ccc(C(=O)c3ccc4c(c3)C(=O)C(C(=O)CCCC(C)C)C4=O)cc2C1=O]

In a similar manner to that of Example 111, the title compound was prepared from 5,5′-carbonylbis(1H-indene-1,3(2H)-dione) (Example 4, 160.0 mg, 0.5 mmol) and 5-methyl-hexanoic acid (CAS number 628-46-6, 195 mg, 1.5 mmol) to provide the title compound as a dark colored bis sodium salt (234 mg, 80%); HRMS (ESI−): calculated for C₃₃H₃₃O₇ m/z [M−H]⁻541.2232, observed 541.2245; ¹H NMR δ 7.87 (d, J=7.0 Hz, 2H), 7.63 (s, 2H), 7.54 (d, J=7.0 Hz, 2H), 2.73 (t, J=7.0 Hz, 4H), 1.57-1.46 (m, 6H), 1.23-1.15 (m, 4H), 0.86 (d, J=6.4 Hz, 12H).

### Example 190—Synthesis of 5-[1,3-dioxo-2-(2-phenylacetyl)-2,3-dihydro-1H-indene-5-carbonyl]-2-(2-phenylacetyl)-2,3-dihydro-1H-indene-1,3-dione

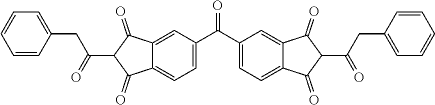 [SMILES: O=C(c1ccc2c(c1)C(=O)C(C(=O)Cc1ccccc1)C2=O)c1ccc2c(c1)C(=O)C(C(=O)Cc1ccccc1)C2=O]

In a similar manner to that of Example 111, the title compound was prepared from 5,5′-carbonylbis(1H-indene-1,3(2H)-dione) (Example 4,160.0 mg, 0.5 mmol) and phenylacetic acid (CAS number 103-82-2, 204 mg, 1.5 mmol) to provide the title compound as a dark colored bis sodium salt (13 mg, 4%); HRMS (ESI−): calculated for C₃₅H₂₁O₇ m/z [M−H]⁻: 553.1293, observed 553.1309; ¹H NMR δ 7.86 (dd, J=1.6, 7.4 Hz, 2H), 7.65 (d, J=1.6 Hz, 2H), 7.55 (d, J=8.0 Hz, 2H), 7.30-7.19 (m, 8H), 7.13 (d, J=7.4 Hz, 2H), 4.11 (s, 4H).

### Example 191—Synthesis of 2-methyl-N-{2-[5-({2-[2-(2-methylpropanamido)acetyl]-1,3-dioxo-2,3-dihydro-1H-inden-5-yl}sulfonyl)-1,3-dioxo-2,3-dihydro-1H-inden-2-yl]-2-oxoethyl}propanamide

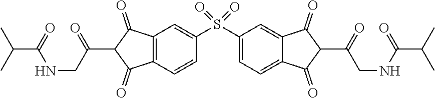 [SMILES: CC(C)C(=O)NCC(=O)C1C(=O)c2ccc(S(=O)(=O)c3ccc4c(c3)C(=O)C(C(=O)CNC(=O)C(C)C)C4=O)cc2C1=O]

In a similar manner to that of Example 136, the title compound was prepared from 5,5′-sulfonylbis(1H-indene-1,3(2H)-dione) (Example 12, 177 mg, 0.5 mmol) and (isobutyrylamino)acetic acid (prepared in Example 187, CAS number 15926-18-8, 218 mg, 1.5 mmol). The bis sodium salt dissolved in methanol and treated with 1N HCl. The resulting precipitate was filtered and dried to provide the title compound (111 mg, 37%). LC/MS (ESI+): R_{f}=4.00 min, (M+H)⁺=609.51; HRMS (ESI−): calculated for C₃₀H₂₇N₂O₁₀S m/z [M−H]−: 607.138325, observed 607.139190; ¹H NMR δ 8.08 (dd, J=2.34, 7.60 Hz, 2H), 7.76 (s, 2H), 7.56 (d, J=7.60 Hz, 2H), 7.45 (t, J=5.30 Hz, 2H), 4.21 (d, J=5.30 Hz, 4H), 2.89-2.96 (m, 2H), 1.00 (d, J=7.00 Hz, 12H).

### Example 192—Synthesis of 2-(5-methylhexanoyl)-5-{[2-(5-methylhexanoyl)-1,3-dioxo-2,3-dihydro-1H-inden-5-yl]oxy}-2,3-dihydro-1H-indene-1,3-dione

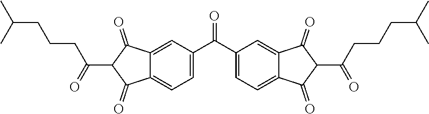 [SMILES: CC(C)CCCC(=O)C1C(=O)c2ccc(C(=O)c3ccc4c(c3)C(=O)C(C(=O)CCCC(C)C)C4=O)cc2C1=O]

A mixture of DMAP (183 mg, 1.50 mmol), 5-methyl-hexanoic acid (CAS number 628-46-6, 193 mg, 1.5 mmol) and 9.0 ml of anhydrous DMF was stirred for 10 min after which a solution had formed. EDCI HCl (CAS number 25952-53-8, 286.5 mg, 1.50 mmol) was added and the contents were stirred for an additional 10 min. 5-[(1,3-Dioxo-2,3-dihydro-1H-inden-5-yl)oxy]-2,3-dihydro-1H-indene-1,3-dione (Example 7, 159 mg, 0.52 mmol) was added and the reaction mixture was stirred overnight. The following day, 15.0 ml of 2N HCl was added and the mixture was stirred for 5 min. The resulting precipitant was filtered from solution. The solid was chromatographed using dichloromethane and methanol with a 1% acetic acid additive to provide the title compound as a yellow-brown solid (15 mg, 9%). HRMS (ESI−): calculated for C₃₂H₃₃O₇ m/z [M−H]−: 529.2232, observed 529.2254; ¹H NMR (CDCl₃) δ 7.88 (dd, J=2.9, 8.2 Hz, 2H), 7.42-7.33 (m, 4H), 2.95 (q, J=8.2 Hz, 4H), 1.79-1.68 (m, 4H), 1.64-1.52 (m, 2H), 1.34-1.25 (m, 4H), 0.89 (dd, J=2.9, 6.4 Hz, 12H).

### Example 193—Synthesis of tert-butyl (2S)-2-[5-({2-[(2S)-1-[(tert-butoxy)carbonyl]pyrrolidine-2-carbonyl]-1,3-dioxo-2,3-dihydro-1H-inden-5-yl}oxy)-1,3-dioxo-2,3-dihydro-1H-indene-2-carbonyl]pyrrolidine-1-carboxylate

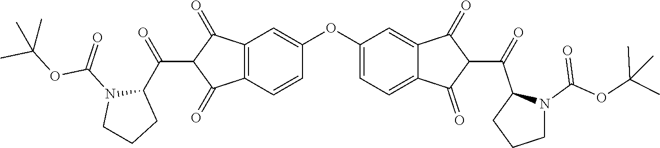 [SMILES: CC(C)(C)OC(=O)N1CCC[C@H]1C(=O)C1C(=O)c2ccc(Oc3ccc4c(c3)C(=O)C(C(=O)[C@@H]3CCCN3C(=O)OC(C)(C)C)C4=O)cc2C1=O]

In a similar manner to that of Example 192, the title compound was prepared from 5,5′-oxybis(1H-indene-1,3(2H)-dione) (Example 7, 159 mg, 0.52 mmol) and N-BOC-L-proline (CAS number 15761-39-4, 323 mg, 1.5 mmol). After the dichloromethane, methanol and 1% acetic acid chromatography the compound was further purified by reverse phase chromatography using acetonitrile and water with 0.1% trifluoroacetic acid to provide the title compound as a yellow solid (15 mg, 4%); HRMS (ESI−): calculated for C₃₈H₃₉N₂O₁₁ m/z [M−H]⁻: 699.2559, observed 699.2547; ¹H NMR (CDCl₃) δ 7.92-7.83 (m, 2H), 7.44-7.29 (m, 4H), 5.56-5.43 (m, 2H), 3.62-3.50 (m, 4H), 2.50-2.38 (m, 2H), 2.06-1.90 (m, 6H), 1.45 (s, 6H), 1.31 (s, 12H).

### Example 194—Synthesis of 5-{1,1,1,3,3,3-hexafluoro-2-[2-(5-methylhexanoyl)-1,3-dioxo-2,3-dihydro-1H-inden-5-yl]propan-2-yl}-2-(5-methylhexanoyl)-2,3-dihydro-1H-indene-1,3-dione

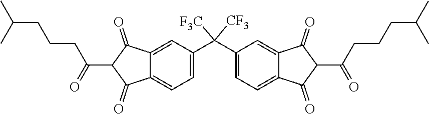 [SMILES: CC(C)CCCC(=O)C1C(=O)c2ccc(C(c3ccc4c(c3)C(=O)C(C(=O)CCCC(C)C)C4=O)(C(F)(F)F)C(F)(F)F)cc2C1=O]

A mixture of DMAP (183 mg, 1.50 mmol), 5-methyl-hexanoic acid (CAS number 628-46-6, 193 mg, 1.5 mmol) and 9.0 ml of anhydrous DMF was stirred for 10 min after which a solution had formed. EDCI HCl (CAS Number 25952-53-8, 286.5 mg, 1.50 mmol) was added and the contents were stirred for an additional 10 min. 5,5′-(perfluoropropane-2,2-diyl)bis(1H-indene-1,3(2H)-dione) (Example 13, 220 mg, 0.5 mmol) was added and the reaction mixture was stirred overnight. The following day, 15.0 ml of 2N HCl was added and the mixture was stirred for 5 min. The resulting precipitant was filtered from solution. The solid was chromatographed using dichloromethane and methanol with a 1% acetic acid additive and the product was further treated with 1N NaOH. The resultant solid was filtered to provide the title compound as a dark purple bis sodium salt (65 mg, 18%). HRMS (ESI−): calculated for C₃₅H₃₃F₆O₆ m/z [M−H]−: 663.2187, observed 663.2179; ¹H NMR δ 7.78-7.32 (m, 6H), 2.81-2.76 (m, 2H), 1.58-1.44 (m, 6H), 1.24-1.13 (m, 6H), 0.82 (d, J=6.4 Hz, 12H).

### Example 195—Synthesis of tert-butyl (2S)-2-[5-(2-{2-[(2S)-1-[(tert-butoxy)carbonyl]pyrrolidine-2-carbonyl]-1,3-dioxo-2,3-dihydro-1H-inden-5-yl}-1,1,1,3,3,3-hexafluoropropan-2-yl)-1,3-dioxo-2,3-dihydro-1H-indene-2-carbonyl]pyrrolidine-1-carboxylate

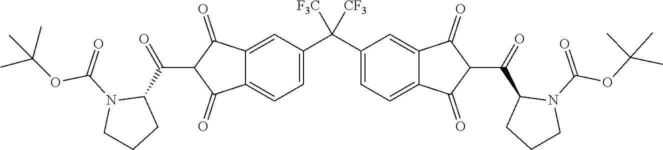 [SMILES: CC(C)(C)OC(=O)N1CCC[C@H]1C(=O)C1C(=O)c2ccc(C(c3ccc4c(c3)C(=O)C(C(=O)[C@@H]3CCCN3C(=O)OC(C)(C)C)C4=O)(C(F)(F)F)C(F)(F)F)cc2C1=O]

In a similar manner to that of Example 194, the title compound was prepared from 5,5′-(perfluoropropane-2,2-diyl)bis(1H-indene-1,3(2H)-dione) (Example 13, 220 mg, 0.5 mmol) and N-BOC-L-proline (CAS number 15761-39-4, 323 mg, 1.5 mmol). After acidification and filtration of the reaction mixture the title compound was isolated as a brown solid (211 mg, 51%); HRMS (ESI−): calculated for C₄₁H₄₀F₆N₂O₁₀ m/z [M−H]−: 833.2514, observed 833.2547; ¹H NMR δ 7.83-7.45 (m, 6H), 5.40-5.30 (m, 2H), 3.41-3.34 (m, 4H), 2.31-2.19 (m, 2H), 1.86-1.75 (m, 6H), 1.38 (s, 6H), 1.22 (s, 12H).

### Example 196—Synthesis of 2-acetyl-5-[2-(2-acetyl-1,3-dioxo-2,3-dihydro-1H-inden-5-yl)ethynyl]-2,3-dihydro-1H-indene-1,3-dione

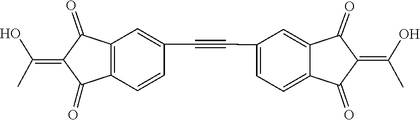 [SMILES: C/C(O)=C1\C(=O)c2ccc(C#Cc3ccc4c(c3)C(=O)/C(=C(/C)O)C4=O)cc2C1=O]

5-[2-(1,3-dioxo-2,3-dihydro-1H-inden-5-yl)ethynyl]-2,3-dihydro-1H-indene-1,3-dione: To a mixture of 4,4′-(Ethyne-1,2-diyl)diphthalic anhydride (CAS number 129808-00-0, 380 mg, 1.0 mmol) and isopropyl acetoacetate (CAS number 542-08-5, 0.35 ml, 2.1 mmol) in acetic anhydride (2.0 ml) was added triethylamine (1.1 ml, 8 mmol). The reaction was heated at 80° C. for 18h then cooled to room temperature. The mixture was diluted with water (10 ml) and treated with concentrated hydrochloric acid (3 ml) and heated at 80° C. for 90 min. The reaction was cooled to room temperature and the solid was collected by filtration, washing with water. After drying under high vacuum, the solid was triturated in dichloromethane and hexanes. The solid was collected by filtration, washed with hexanes to obtain primarily 5-[2-(1,3-dioxo-2,3-dihydro-1H-inden-5-yl)ethynyl]-2,3-dihydro-1H-indene-1,3-dione as a brown solid (400 mg). ¹H NMR δ 8.17-8.11 (m, 4H), 7.99 (d, J=7.0 Hz, 2H), 3.32 (s, 4H). This solid was used in the next step without further purification.

A mixture of DMAP (122 mg, 1.0 mmol), acetic acid (55 μL, 0.96 mmol) and 6 ml of anhydrous DMF was stirred for 10 min after which a solution had formed. EDCI HCl (CAS Number 25952-53-8, 191 mg, 1.0 mmol) was added and the contents were stirred for an additional 10 min. 55-[2-(1,3-dioxo-2,3-dihydro-1H-inden-5-yl)ethynyl]-2,3-dihydro-1H-indene-1,3-dione as a brown solid (100 mg) was added and the reaction mixture was stirred overnight. After acidification and filtration of the reaction mixture the title compound was isolated as a brown solid (26 mg, 20%). HRMS (ESI−): calculated for C₂₄H₁₃O₆ m/z [M−H]−: 397.070807, observed 397.071762; ¹H NMR δ 8.02-7.95 (m, 4H), 7.87-7.74 (m, 2H), 2.53 (s, 6H).

### Example 209—Synthesis of tert-butyl (2S)-2-{2′-[(2S)-1-[(tert-butoxy)carbonyl]pyrrolidine-2-carbonyl]-1,1′,3,3′-tetraoxo-1H,1′H,2H,2′H,3H,3′H-[5,5′-biindene]-2-carbonyl}pyrrolidine-1-carboxylate

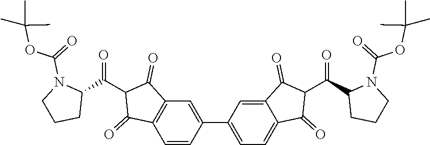

A mixture of DMAP (183 mg, 1.50 mmol), BOC-L-proline (CAS number 15761-39-4, 322 mg, 1.50 mmol) and 9.0 ml of anhydrous DMF were stirred for 10 min. EDCI*HCl (CAS number 25952-53-8, 286.5 mg, 1.50 mmol) was added and the contents were stirred for an additional 10 min. 1H,1′H,2H,2′H,3H,3′H-[5,5′-Biindene]-1,1′,3,3′-tetrone (Example 75, 145.0 mg, 0.5 mmol) was added and the contents were stirred overnight. The following day, 15.0 ml of 2N HCl was added and the resulting precipitant was filtered. The solid was washed once with 5 ml of 2N HCl and 5 ml of water. The dried solid was milled into a fine powder and was treated with 10 ml of 1N NaOH and 5 ml of methylene chloride. This biphasic suspension was shaken vigorously and filtered to provide the title compound as a dark colored bis sodium salt (197.2 mg, 54%). LCMS (ESI+): R_{f}=7.99, (M−H)−; HRMS (ESI−): calculated for C₃₈H₃₉N₂O₁₀ m/z [M−H]⁻: 683.2610, observed 683.2601; ¹H NMR δ: 7.78-7.88 (m, 2H), 7.63 (s, 2H), 7.44-7.55 (m, 2H), 5.24-5.60 (m, 2H), 3.39-3.55 (m, 2H), 2.00-2.37 (m, 4H), 1.57-1.90 (m, 4H), 1.39 (s, 8H), 1.24 (s, 10H).

### Example 210—Synthesis of tert-butyl (4R)-4-{2′-[(4R)-3-[(tert-butoxy)carbonyl]-1,3-thiazolidine-4-carbonyl]-1,1′,3,3′-tetraoxo-1H,1′H,2H,2′H,3H,3′H-[5,5′-biindene]-2-carbonyl}-1,3-thiazolidine-3-carboxylate

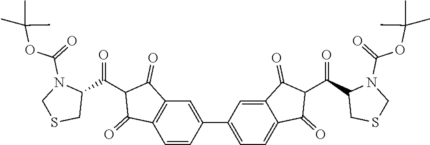

In a similar manner to that of Example 209, the title compound was prepared from 1H,1′H,2H,2′H,3H,3′H-[5,5′-biindene]-1,1′,3,3′-tetrone (Example 75, 145.0 mg, 0.5 mmol) and (4R)-3-(tert-butoxycarbonyl)-1,3-thiazolidine-4-carboxylic acid (CAS number 51077-16-8, 349.6 mg, 1.50 mmol) to provide the title compound as a dark colored bis sodium salt (221.0 mg, 58% yield). LCMS (ESI+): R_{f}=7.97, (M−H)⁻; HRMS (ESI−): calculated for C₃₆H₃₅N₂O₁₀S₂ m/z [M−H]⁻: 719.1739, observed 719.1724; ¹H NMR δ: 7.79-7.91 (m, 2H), 7.62-7.70 (m, 2H), 7.51 (d, J=1.0 Hz, 2H), 5.63-5.87 (m, 2H), 4.54-4.79 (m, 2H), 4.27-4.49 (m, 2H), 3.44-3.61 (m, 4H), 1.42 (s, 8H), 1.28 (s, 10H).

### Example 211—Synthesis of tert-butyl 6-(2′-{2-[(tert-butoxy)carbonyl]-2-azaspiro[3.3]heptane-6-carbonyl}-1,1′,3,3′-tetraoxo-1H,1′H,2H,2′H,3H,3′H-[5,5′-biindene]-2-carbonyl)-2-azaspiro[3.3]heptane-2-carboxylate

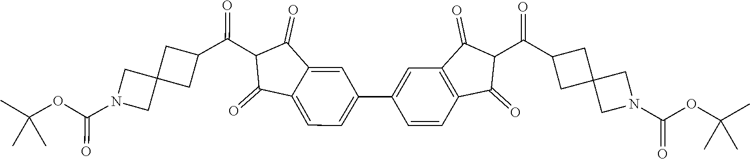 [SMILES: CC(C)(C)OC(=O)N1CC2(CC(C(=O)C3C(=O)c4ccc(-c5ccc6c(c5)C(=O)C(C(=O)C5CC7(C5)CN(C(=O)OC(C)(C)C)C7)C6=O)cc4C3=O)C2)C1]

In a similar manner to that of Example 209, the title compound was prepared from 1H,1′H,2H,2′H,3H,3′H-[5,5′-biindene]-1,1′,3,3′-tetrone (Example 75, 145.0 mg, 0.5 mmol) and 2-(tert-butoxycarbonyl)-2-azaspiro[3.3]heptane-6-carboxylic acid (CAS number 1211526-53-2, 361.6 mg, 1.50 mmol) to provide the title compound as a dark colored bis sodium salt (278.2 mg, 71% yield). LCMS (ESI+): R_{f}=8.78, (M−H)⁻; HRMS (ESI−): calculated for C₄₂H₄₃N₂O₁₀ m/z [M−H]⁻: 735.2923, observed 735.2893; ¹H NMR δ: 7.73-7.82 (m, 2H), 7.57 (s, 2H), 7.43 (d, J=7.6 Hz, 2H), 3.99-4.21 (m, 2H), 3.86 (br s, 4H), 3.69 (br s, 4H), 2.10-2.36 (m, 8H), 1.34 (s, 18H).

### Example 212—Synthesis of tert-butyl (2S)-2-{2′-[(2S)-1-[(tert-butoxy)carbonyl]piperidine-2-carbonyl]-1,1′,3,3′-tetraoxo-1H,1′H,2H,2′H,3H,3′H-[5,5′-biindene]-2-carbonyl}piperidine-1-carboxylate

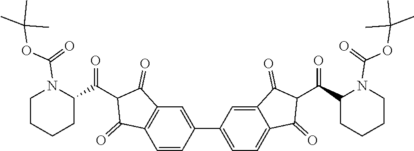

In a similar manner to that of Example 209, the title compound was prepared from 1H,1′H,2H,2′H,3H,3′H-[5,5′-biindene]-1,1′,3,3′-tetrone (Example 75, 145.0 mg, 0.5 mmol) and (2S)-1-(tert-butoxycarbonyl)piperidine-2-carboxylic acid (CAS number 26250-84-0, 343.6 mg, 1.50 mmol) to provide the title compound as a dark colored bis sodium salt (289.0 mg, 76% yield. LCMS (ESI+): R_{f}=6.97, (M−H)⁻; HRMS (ESI−): calculated for C₄₀H₄₃N₂O₁₀ m/z [M−H]⁻: 711.292319, observed 711.291553; ¹H NMR δ: 7.75-7.88 (m, 2H), 7.60 (s, 2H), 7.45 (d, J=7.5 Hz, 2H), 5.22-5.81 (m, 2H), 3.64-3.98 (m, 2H), 3.40-3.63 (m, 2H), 3.13-3.27 (m, 2H), 1.78-2.08 (m, 2H), 1.58 (br d, J=2.8 Hz, 4H), 1.05-1.48 (m, 22H).

### Example 213—Synthesis of methyl (2S)-2-{2′-[(2S)-1-(methoxycarbonyl)pyrrolidine-2-carbonyl]-1,1′,3,3′-tetraoxo-1H,1′H,2H,2′H,3H,3′H-[5,5′-biindene]-2-carbonyl}pyrrolidine-1-carboxylate

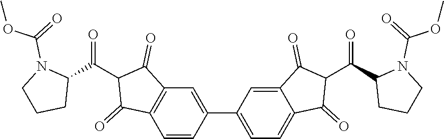

In a similar manner to that of Example 209, the title compound was prepared from 1H,1′H,2H,2′H,3H,3′H-[5,5′-biindene]-1,1′,3,3′-tetrone (Example 75, 145.0 mg, 0.5 mmol) and (2S)-1-(methoxycarbonyl)pyrrolidine-2-carboxylic acid (CAS number 74761-41-4, 259.5 mg, 1.50 mmol) to provide the title compound as a dark colored bis sodium salt (197.2 mg, 61% yield). LCMS (ESI+): R_{f}=5.31, (M−H)⁻; HRMS (ESI−): calculated for C₃₂H₂₇N₂O₁₀ m/z [M−H]⁻: 599.167119, observed 599.166091; ¹H NMR δ: 7.77-7.91 (m, 2H), 7.63 (s, 2H), 7.43-7.53 (m, 2H), 5.28-5.54 (m, 2H), 3.52 (s, 3H), 3.42 (s, 3H), 3.35-3.39 (m, 2H), 3.33 (m, 2H), 2.24 (m, 2H), 1.72 (m, 4H), 1.06-1.39 (m, 2H).

### Example 214—Synthesis of tert-butyl (2S,5R)-2-{2′-[(2S,5R)-1-[(tert-butoxy)carbonyl]-5-methylpyrrolidine-2-carbonyl]-1,1′,3,3′-tetraoxo-1H,1′H,2H,2′H,3H,3′H-[5,5′-biindene]-2-carbonyl}-5-methylpyrrolidine-1-carboxylate

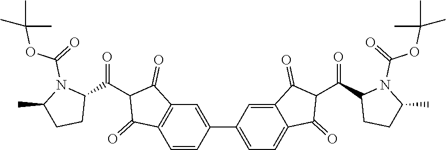

In a similar manner to that of Example 209, the title compound was prepared from 1H,1′H,2H,2′H,3H,3′H-[5,5′-biindene]-1,1′,3,3′-tetrone (Example 75, 145.0 mg, 0.5 mmol) and (2S,5R)-1-(tert-butoxycarbonyl)-5-methylpyrrolidine-2-carboxylic acid (CAS number 160033-52-3, 343.6 mg, 1.50 mmol) to provide the title compound as a dark colored bis sodium salt (255.2 mg, 67%). LCMS (ESI+): R_{f}=8.97, (M−H)⁻; HRMS (ESI−): calculated for C₄₀H₄₃N₂O₁₀ m/z [M−H]⁻: 711.292380, observed 711.292319; ¹H NMR δ: 7.74-7.91 (m, 2H), 7.60 (s, 2H), 7.39-7.53 (m, 2H), 5.47 (m, 2H), 3.65-3.97 (m, 2H), 2.10 (s, 2H), 1.88 (s, 2H), 1.61 (s, 2H), 1.36 (s, 8H), 1.20 (s, 16H).

### Example 215—Synthesis of tert-butyl (2S,4R)-2-{2′-[(2S,4R)-1-[(tert-butoxy)carbonyl]-4-fluoropyrrolidine-2-carbonyl]-1,1′,3,3′-tetraoxo-1H,1′H,2H,2′H,3H,3′H-[5,5′-biindene]-2-carbonyl}-4-fluoropyrrolidine-1-carboxylate

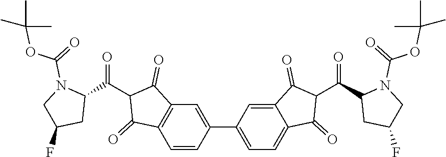

In a similar manner to that of Example 209, the title compound was prepared from 1H,1′H,2H,2′H,3H,3′H-[5,5′-biindene]-1,1′,3,3′-tetrone (Example 75, 145.0 mg, 0.5 mmol) and (2S, 4R)-1-[(tert-butoxy)carbonyl]-4-fluoropyrrolidine-2-carboxylic acid (CAS number 203866-14-2, 349.5 mg, 1.50 mmol) to provide the title compound as a dark colored bis sodium salt (225.2 mg, 59%). LCMS (ESI+): R_{f}=7.28, (M−H)⁻; HRMS (ESI−): calculated for C₃₇H₃₅N₂O₁₅ m/z [M−H]⁻: 747.201985, observed 747.204292; ¹H NMR δ: 7.76-7.89 (m, 2H), 7.61 (s, 2H), 7.42-7.51 (m, 2H), 5.40-5.78 (m, 2H), 5.22-5.38 (m, 1H), 5.03-5.20 (m, 1H), 3.56-3.84 (m, 2H), 3.41-3.55 (m, 2H), 3.10-3.24 (m, 2H), 1.60-2.03 (m, 2H), 1.37 (s, 8H), 1.28 (s, 10H).

### Example 216—Synthesis of tert-butyl (1R,5S,6R)-6-{2′-[(1R,5S,6R)-3-[(tert-butoxy)carbonyl]-3-azabicyclo[3.1.0]hexane-6-carbonyl]-1,1′,3,3′-tetraoxo-1H,1′H,2H,2′H,3H,3′H-[5,5′-biindene]-2-carbonyl}-3-azabicyclo[3.1.0]hexane-3-carboxylate

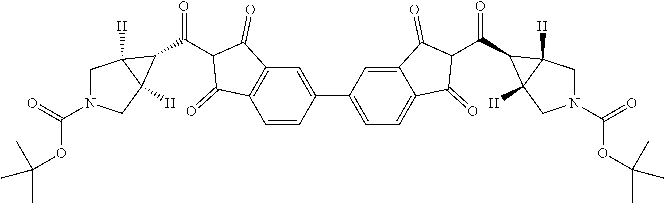 [SMILES: CC(C)(C)OC(=O)N1C[C@@H]2[C@H](C1)[C@H]2C(=O)C1C(=O)c2ccc(-c3ccc4c(c3)C(=O)C(C(=O)[C@H]3[C@@H]5CN(C(=O)OC(C)(C)C)C[C@@H]53)C4=O)cc2C1=O]

In a similar manner to that of Example 209, the title compound was prepared from 1H,1′H,2H,2′H,3H,3′H-[5,5′-biindene]-1,1′,3,3′-tetrone (Example 75) and (1R,5S,6R)-3-(tert-butoxycarbonyl)-3-azabicyclo[3.1.0]hexane-6-carboxylic acid (CAS number 927679-54-7) to provide a dark solid bis-sodium salt. HRMS (ESI−): calculated for C₄₀H₃₉N₂O₁₀ m/z [M−H]⁻: 707.261019, observed 707.259466.

### Example 217—Synthesis of methyl (1R,2R)-2-(5-{2-[(1R,2R)-2-(methoxycarbonyl) cyclopropanecarbonyl]-1,3-dioxo-2,3-dihydro-1H-indene-5-carbonyl}-1,3-dioxo-2,3-dihydro-1H-indene-2-carbonyl)cyclopropane-1-carboxylate

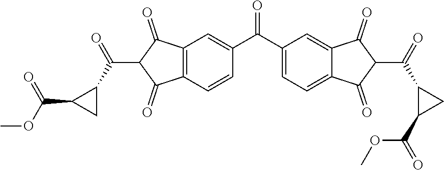 [SMILES: COC(=O)[C@@H]1C[C@H]1C(=O)C1C(=O)c2ccc(C(=O)c3ccc4c(c3)C(=O)C(C(=O)[C@@H]3C[C@H]3C(=O)OC)C4=O)cc2C1=O]

In a similar manner to that of Example 111, the title compound was prepared from 5,5′-carbonylbis(1H-indene-1,3(2H)-dione) (Example 4) and (1R,2R)-1,2-cyclopropanedicarboxylic acid, 1-methyl ester (CAS number 88335-97-1) to provide the title compound as a dark colored bis sodium salt. HRMS (ESI−): calculated for C₃₁H₂₁O₁₁ m/z [M−H]⁻: 569.108935, observed 569.111646.

### Example 218—Synthesis of methyl (1R,2R)-2-[5-({2-[(1R,2R)-2-(methoxycarbonyl) cyclopropanecarbonyl]-1,3-dioxo-2,3-dihydro-1H-inden-5-yl}sulfonyl)-1,3-dioxo-2,3-dihydro-1H-indene-2-carbonyl]cyclopropane-1-carboxylate

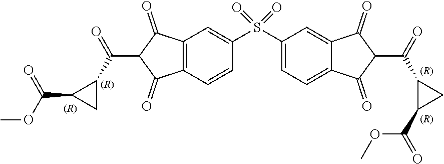

In a similar manner to that of Example 136, the title compound was prepared from 5,5′-sulfonylbis(1H-indene-1,3(2H)-dione) (Example 12) and (1R,2R)-1,2-cyclopropanedicarboxylic acid, 1-methyl ester (CAS number 88335-97-1) to provide the title compound as a dark colored bis sodium salt. HRMS (ESI−): calculated for C₃₀H₂₁O₁₂S m/z [M−H]⁻: 605.075921, observed 605.075079.

### Example 219—Synthesis of methyl 4-{52-(4-methoxy-3,3-dimethyl-4-oxobutanoyl)-1,3-dioxo-2,3-dihydro-1H-indene-5-carbonyl]-1,3-dioxo-2,3-dihydro-1H-inden-2-yl}-2,2-dimethyl-4-oxobutanoate

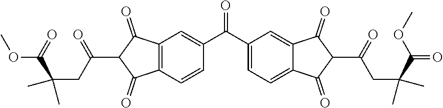

In a similar manner to that of Example 111, the title compound was prepared from 5,5′-carbonylbis(1H-indene-1,3(2H)-dione) (Example 4) and butanedioic acid, 2,2-dimethyl-, 1-methyl ester (CAS number 32980-26-0) to provide the title compound as a dark colored bis sodium salt. HRMS (ESI−): calculated for C₃₃H₂₉O₁₁ m/z [M−H]⁻: 601.170367, observed 601.171535.

### Example 220—Synthesis of 2-[(3aS,4S,6R,6aS)-6-methoxy-2,2-dimethyl-tetrahydro-2H-furo[3,4-d][1,3]dioxole-4-carbonyl]-5-({2-[(3aS,4S,6R,6aS)-6-methoxy-2,2-dimethyl-tetrahydro-2H-furo[3,4-d][1,3]dioxole-4-carbonyl]-1,3-dioxo-2,3-dihydro-1H-inden-5-yl}sulfonyl)-2,3-dihydro-1H-indene-1,3-dione

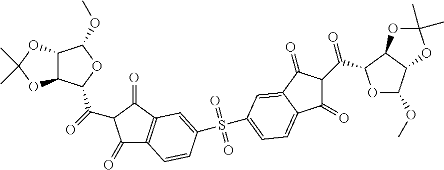

In a similar manner to that of Example 136, the title compound was prepared from 5,5′-sulfonylbis(1H-indene-1,3(2H)-dione) (Example 12) and methyl 2,3-O-(1-methylethylidene)-β-D-ribofuranosiduronic acid (CAS number 54622-95-6) to provide the title compound as a dark colored bis sodium salt. HRMS (ESI−): calculated for C₃₆H₃₃O₁₆S m/z [M−H]753.149480, observed 753.150035.

### Example 221—Synthesis of tert-butyl (4S)-4-[5-({2-[(4S)-3-[(tert-butoxy)carbonyl]-1,3-thiazolidine-4-carbonyl]-1,3-dioxo-2,3-dihydro-1H-inden-5-yl}sulfonyl)-1,3-dioxo-2,3-dihydro-1H-indene-2-carbonyl]-1,3-thiazolidine-3-carboxylate

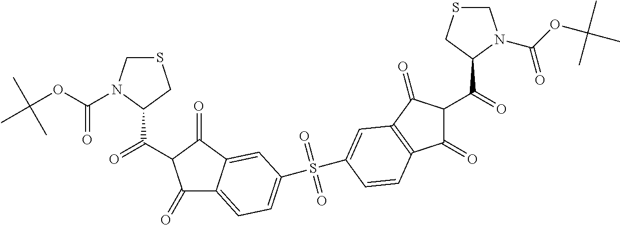 [SMILES: CC(C)(C)OC(=O)N1CSC[C@@H]1C(=O)C1C(=O)c2ccc(S(=O)(=O)c3ccc4c(c3)C(=O)C(C(=O)[C@H]3CSCN3C(=O)OC(C)(C)C)C4=O)cc2C1=O]

In a similar manner to that of Example 136, the title compound was prepared from 5,5′-sulfonylbis(1H-indene-1,3(2H)-dione) (Example 12) and (4S)-3-(tert-butoxycarbonyl)-1,3-thiazolidine-4-carboxylic acid (CAS number 63091-82-7) to provide the title compound as a dark colored bis sodium salt. HRMS (ESI−): calculated for C₃₆H₃₅N₂O₁₂S₃ m/z [M−H]783.135761, observed 783.135923.

### Example 222.—Synthesis of (1R,2R)-2-(5-{2-[(1R,2R)-2-carboxycyclopropanecarbonyl]-1,3-dioxo-2,3-dihydro-1H-indene-5-carbonyl}-1,3-dioxo-2,3-dihydro-1H-indene-2-carbonyl)cyclopropane-1-carboxylic acid

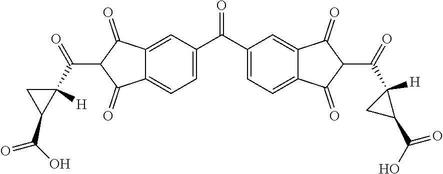 [SMILES: O=C(c1ccc2c(c1)C(=O)C(C(=O)[C@H]1C[C@@H]1C(=O)O)C2=O)c1ccc2c(c1)C(=O)C(C(=O)[C@H]1C[C@@H]1C(=O)O)C2=O]

72.0 mg of of methyl (1 R,2R)-2-(5-{2-[(1R,2R)-2-(methoxycarbonyl) cyclopropanecarbonyl]-1,3-dioxo-2,3-dihydro-1H-indene-5-carbonyl}-1,3-dioxo-2,3-dihydro-1H-indene-2-carbonyl)cyclopropane-1-carboxylate (example 217) was added to 5 ml of MeOH. Then an excess of 1 N NaOH (10 ml) was added and stirred overnight. There was then added a large excess of 2 N HCL (20 ml) and a solid came from solution which was filtered and dried under high vacc. There was obtained 55.2 mg of the titled compound corresponding to an 80% yield. HRMS (ESI−): calculated for C₂₉H₁₇O₁₁ m/z [M−H]541.080315, observed 541.077635. LCMS ¹H NMR (DMSO-d₆) δ: 8.13 (dd, J=7.6, 1.8 Hz, 2H), 7.79 (s, 2H), 7.60 (d, J=7.7 Hz, 2H), 3.83 (ddd, J=9.0, 5.7, 3.8 Hz, 2H), 3.50-3.67 (s, 6H), 1.91 (ddd, J=8.5, 5.2, 3.8 Hz, 2H), 1.10-1.37 (m, 4H)

### Example 223—Synthesis of (1R,2R)-2-[5-({2-[(1R,2R)-2-carboxycyclopropanecarbonyl]-1,3-dioxo-2,3-dihydro-1H-inden-5-yl}sulfonyl)-1,3-dioxo-2,3-dihydro-1H-indene-2-carbonyl]cyclopropane-1-carboxylic acid

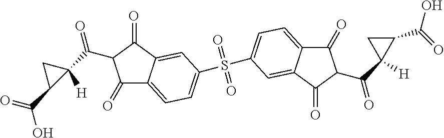 [SMILES: O=C1c2ccc(S(=O)(=O)c3ccc4c(c3)C(=O)C(C(=O)[C@H]3C[C@@H]3C(=O)O)C4=O)cc2C(=O)C1C(=O)[C@H]1C[C@@H]1C(=O)O]

In a similar manner to that of Example 222, there was taken 91.0 mg of methyl (1 R,2R)-2-[5-({2-[(1R,2R)-2-(methoxycarbonyl) cyclopropanecarbonyl]-1,3-dioxo-2,3-dihydro-1H-inden-5-yl}sulfonyl)-1,3-dioxo-2,3-dihydro-1H-indene-2-carbonyl]cyclopropane-1-carboxylate (Example 218). There was obtained 68.0 mg of the titled compound corresponding to a 78% yield. HRMS (ESI−): calculated for C₂₈H₁₇O₁₂S m/z [M−H]577.044621, observed 577.047211. LCMS RF=3.83, ¹H NMR (DMSO-d₆) δ: 8.19-8.28 (m, 2H), 7.95 (s, 2H), 7.71 (d, J=7.7 Hz, 2H), 3.60-3.76 (m, 2H), 1.83-2.03 (m, 2H), 1.16-1.41 (m, 4H)

### Example 224 Synthesis of tert-butyl (2S,4S)-2-[5-({2-[(2S,4S)-1-[(tert-butoxy)carbonyl]-4-methylpyrrolidine-2-carbonyl]-1,3-dioxo-2,3-dihydro-1H-inden-5-yl}sulfonyl)-1,3-dioxo-2,3-dihydro-1H-indene-2-carbonyl]-4-methylpyrrolidine-1-carboxylate

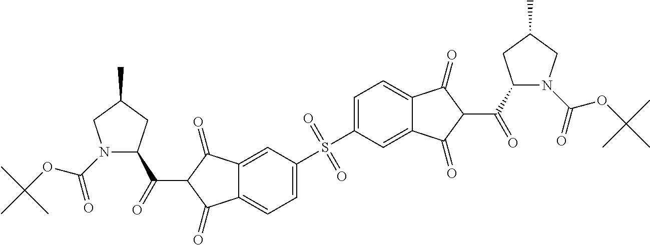 [SMILES: C[C@H]1C[C@@H](C(=O)C2C(=O)c3ccc(S(=O)(=O)c4ccc5c(c4)C(=O)C(C(=O)[C@@H]4C[C@H](C)CN4C(=O)OC(C)(C)C)C5=O)cc3C2=O)N(C(=O)OC(C)(C)C)C1]

In a similar manner to Example 136, the title compound was prepared from 5,5′-sulfonylbis(1H-indene-1,3(2H)-dione) (Example 12) and (2S,4S)-1-(tert butoxycarbonyl)-4-methylpyrrolidine-2-carboxylic acid (CAS no 364750-81-2). SM=0.177, acid=343.58 mg There was obtained 308 mg of material corresponding to a 79% yield. HRMS (ESI−): calculated for C₄₀H₄₃N₂O₁₂S m/z [M−H]775.254219, observed 775.253922. LCMS ¹H NMR (DMSO-d₆) δ: 8.03-8.17 (m, 2H), 7.73-7.78 (m, 2H), 7.52-7.63 (m, 2H), 5.11-5.45 (m, 2H), 3.48-3.74 (m, 2H), 2.60-2.80 (m, 2H), 2.29-2.46 (m, 2H), 1.98-2.18 (m, 4H), 1.34 (s, 6H), 1.14 (s, 18H)

### Example 225—Synthesis of methyl 5-{5-[2-(5-methoxy-5-oxopentanoyl)-1,3-dioxo-2,3-dihydro-1H-indene-5-carbonyl]-1,3-dioxo-2,3-dihydro-1H-inden-2-yl}-5-oxopentanoate

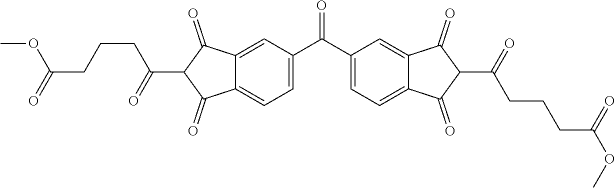 [SMILES: COC(=O)CCCC(=O)C1C(=O)c2ccc(C(=O)c3ccc4c(c3)C(=O)C(C(=O)CCCC(=O)OC)C4=O)cc2C1=O]

In a similar manner to that of Example 111, the title compound was prepared from 5,5′-carbonylbis(1H-indene-1,3(2H)-dione) (Example 4) and pentanedioic acid, 1-methyl ester (CAS no 1501-27-5) to provide the title compound as a dark colored bis sodium salt. HRMS (ESI−): calculated for C₃₁H₂₅O₁₁ m/z [M−H]573.140235, observed 573.140235. LCMS ¹H NMR (DMSO-d₆) δ: 7.82-7.90 (m, 2H), 7.59-7.65 (m, 2H), 7.50-7.58 (m, 2H), 3.56 (s, 6H), 2.66-2.83 (m, 4H), 2.19-2.41 (m, 4H), 1.62-1.84 (m, 4H).

### Example 226—Synthesis of methyl 5-[2′-(5-methoxy-5-oxopentanoyl)-1,1′,3,3′-tetraoxo-1H,1′H,2H,2′H,3H,3′H-[5,5′-biinden]-2-yl]-5-oxopentanoate

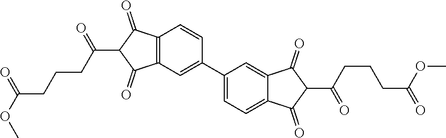 [SMILES: COC(=O)CCCC(=O)C1C(=O)c2ccc(-c3ccc4c(c3)C(=O)C(C(=O)CCCC(=O)OC)C4=O)cc2C1=O]

In a similar manner to that of Example 209, the title compound was prepared from 1H,1′H,2H,2′H,3H,3′H-[5,5′-biindene]-1,1′,3,3′-tetrone (Example 75) and pentanedioic acid, 1-methyl ester (CAS no 1501-27-5) to provide the title compound as a dark colored bis sodium salt. HRMS (ESI−): calculated for C₃₀H₂₅O₁₀ m/z [M−H]545.145321, observed 545.144722. LCMS ¹H NMR (DMSO-d₆) δ: 7.75-7.84 (m, 2H), 7.59 (s, 2H), 7.45 (d, J=7.6 Hz, 2H), 3.56 (s, 6H), 2.67-2.83 (m, 4H), 2.23-2.38 (m, 4H), 1.67-1.83 (m, 4H)

### Example 227—Synthesis of 5-[2′-(4-carboxybutanoyl)-1,1′,3,3′-tetraoxo-1H,1′H,2H, 2′H,3H,3′H-[5,5′-biinden]-2-yi]-5-oxopentanoic acid

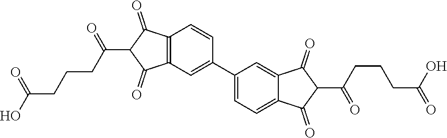 [SMILES: O=C(O)CCCC(=O)C1C(=O)c2ccc(-c3ccc4c(c3)C(=O)C(C(=O)CCCC(=O)O)C4=O)cc2C1=O]

In a similar manner to that of Example 222, the title compound was obtained from methyl 5-[2′-(5-methoxy-5-oxopentanoyl)-1,1′,3,3′-tetraoxo-1H,1′H,2H,2′H,3H,3′H-[5,5′-biinden]-2-yl]-5-oxopentanoate (Example 226) to provide 83 mg of the titled compound corresponding to a 88% yield. ¹H NMR (DMSO-d₆) δ: 8.08 (dd, J=7.7, 1.4 Hz, 2H), 7.93 (s, 2H), 7.87 (d, J=7.6 Hz, 2H), 2.94 (br t, J=7.4 Hz, 4H), 2.29 (t, J=7.4 Hz, 4H), 1.65-1.96 (m, 4H).

### Example 228—Synthesis of tert-butyl N-[2-(5-{2-[2-(2-{[(tert-butoxy)carbonyl]amino}acetyl)-1,3-dioxo-2,3-dihydro-1H-inden-5-yl]-1,1,1,3,3,3-hexafluoropropan-2-yl}-1,3-dioxo-2,3-dihydro-1H-inden-2-yl)-2-oxoethyl]carbamate

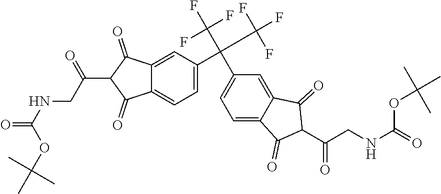 [SMILES: CC(C)(C)OC(=O)NCC(=O)C1C(=O)c2ccc(C(c3ccc4c(c3)C(=O)C(C(=O)CNC(=O)OC(C)(C)C)C4=O)(C(F)(F)F)C(F)(F)F)cc2C1=O]

In a similar manner to that of Example 194, the title compound was prepared from 5,5′-(perfluoropropane-2,2-diyl)bis(1H-indene-1,3(2H)-dione) (Example 13) and 2-(tert-butoxy)-carbonylamino acetic acid (CAS number 4530-20-5). After acidification and filtration of the reaction mixture the title compound was isolated as a brown solid corresponding to a 73% yield. RF=5.68, ¹H NMR (DMSO-d₆) δ: 7.77-7.86 (m, 2H), 7.62 (s, 2H), 7.47 (br d, J=7.6 Hz, 2H), 4.04-4.31 (m, 4H), 1.38 (s, 18H).

### Example 229—Synthesis of 2-(2-ethoxyacetyl)-5-[2-(2-ethoxyacetyl)-1,3-dioxo-2,3-dihydro-1H-indene-5-carbonyl]-2,3-dihydro-1H-indene-1,3-dione

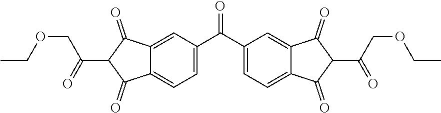 [SMILES: CCOCC(=O)C1C(=O)c2ccc(C(=O)c3ccc4c(c3)C(=O)C(C(=O)COCC)C4=O)cc2C1=O]

To a solution of dimethylaminopyridine (DMAP, 183 mg, 1.50 mmol), ethoxy acetic acid (CAS number 627-03-2, 229.5 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15.0 ml of 2N HCl was added and the resulting precipitant was filtered. The solid was washed once with 5 ml of 2N HCl and 5 ml of water. The dried solid was milled into a fine powder and was treated with 10 ml of 1 N NaOH and 5 ml of methylene chloride. This biphasic suspension was shaken vigorously and filtered to provide the title compound dark solid bis-sodium salt (85.8 mg, 32%). LCMS (ESI+): R_{f}=5.08, (M+H)⁺=491.44; HRMS (ESI−): calculated for C₂₇H₂₁O₉ m/z [M−H]⁻: 489.1191, observed 489.1180; ¹H NMR 300 MHz DMSO δ: 7.83-7.88 (m, 1H), 7.62 (s, 1H), 7.51-7.55 (m, 1H), 3.44-3.54 (m, 2H), 1.09-1.18 (m, 3H).

### Example 230—Synthesis of methyl 4-{5-[2-(4-methoxy-4-oxobutanoyl)-1,3-dioxo-2,3-dihydro-1H-indene-5-carbonyl]-1,3-dioxo-2,3-dihydro-1H-inden-2-yl}-4-oxobutanoate

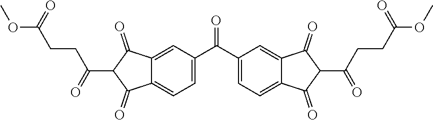

To a solution of dimethylaminopyridine (DMAP, 183 mg, 1.50 mmol), monomethyl succinate (CAS number 3878-55-5, 198.2 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of saturated aqueous NaHCO₃ and 5 ml of methylene chloride. This biphasic suspension was shaken vigorously and filtered to provide the title compound dark solid bis-sodium salt (259.3 mg, 93%). LCMS (ESI+): R_{f}=5.61, (M+H)⁺=547.37; HRMS (ESI−): calculated for C₂₉H₂₁O₁₁ m/z [M−H]⁻: 545.1089, observed 545.1096; ¹H NMR 300 MHz DMSO δ: 7.84-7.90 (m, 1H), 7.64 (s, 1H), 7.55 (d, J=7.03 Hz, 1H), 3.56 (s, 3H), 3.05 (t, J=7.03 Hz, 2H).

### Example 231—Synthesis of 2-[2-(tert-butoxy)acetyl]-5-({2-[2-(tert-butoxy)acetyl]-1,3-dioxo-2,3-dihydro-1H-inden-5-yl}sulfonyl)-2,3-dihydro-1H-indene-1,3-dione

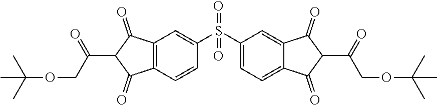 [SMILES: CC(C)(C)OCC(=O)C1C(=O)c2ccc(S(=O)(=O)c3ccc4c(c3)C(=O)C(C(=O)COC(C)(C)C)C4=O)cc2C1=O]

To a solution of dimethylaminopyridine (DMAP, 183 mg, 1.50 mmol), tertbutoxy acetic acid (CAS number 13211-32-0, 198.2 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred ambient temperature. Once the reaction was deemed complete by LCMS, 15.0 ml of 2N HCl was added and the resulting precipitant was filtered. The solid was washed once with 5 ml of 2N HCl and 5 ml of water. The dried solid was milled into a fine powder and was treated with 10 ml of saturated aqueous NaHCO₃ and 5 ml of methylene chloride. This biphasic suspension was shaken vigorously and filtered to provide the title compound dark solid bis-sodium salt (262.4 mg, 84%). LCMS (ESI+): R_{f}=6.13, (M+H)+−2 tert butyl=471.55; HRMS (ESI−): calculated for C₃₀H₂₉O₁₀S m/z [M−H]⁻: 581.1487, observed 581.1494; ¹H NMR 300 MHz DMSO δ: 8.06-8.11 (m, 1H), 7.73-7.76 (m, 1H), 7.53-7.58 (m, 1H), 4.37-4.45 (m, 2H), 1.08-1.17 (m, 9H).

### Example 232—Synthesis of 2-[2-(cyclohexyloxy)acetyl]-5-({2-[2-(cyclohexyloxy)acetyl]-1,3-dioxo-2,3-dihydro-1H-inden-5-yl}sulfonyl)-2,3-dihydro-1H-indene-1,3-dione

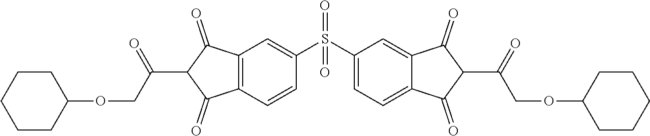 [SMILES: O=C(COC1CCCCC1)C1C(=O)c2ccc(S(=O)(=O)c3ccc4c(c3)C(=O)C(C(=O)COC3CCCCC3)C4=O)cc2C1=O]

To a solution of dimethylaminopyridine (DMAP, 183 mg, 1.50 mmol), 2-(cyclohexyloxy)acetic acid (CAS number 71995-54-5, 237.3 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of saturated aqueous NaHCO₃ and 5 ml of methylene chloride. This biphasic suspension was shaken vigorously and filtered to provide the title compound dark solid bis-sodium salt (262.4 mg, 84%). LCMS (ESI+): R_{f}=7.43, (M+H)+=635.74; HRMS (ESI−): calculated for C₃₄H₃₃O₁₀S m/z [M−H]⁻: 633.1800, observed 633.1790; ¹H NMR 300 MHz DMSO δ: 7.93-7.96 (m, 1H), 7.71-7.74 (m, 1H), 7.52-7.56 (m, 1H), 4.43-4.48 (m, 2H), 1.75-1.90 (m, 4H), 1.56-1.70 (m, 4H), 1.10-1.23 (m, 2H), 1.10-1.23 (m, 9H).

### Example 233 Synthesis of tert-butyl 2-(5-{[2-({4-[(tert-butoxy)carbonyl]morpholin-2-yl}(hydroxy)methyl)-1,3-dioxo-2,3-dihydro-1H-inden-5-yl]sulfonyl}-1,3-dioxo-2,3-dihydro-1H-indene-2-carbonyl)morpholine-4-carboxylate

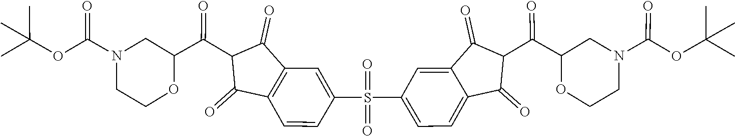 [SMILES: CC(C)(C)OC(=O)N1CCOC(C(=O)C2C(=O)c3ccc(S(=O)(=O)c4ccc5c(c4)C(=O)C(C(=O)C4CN(C(=O)OC(C)(C)C)CCO4)C5=O)cc3C2=O)C1]

To a solution of dimethylaminopyridine (DMAP, 183 mg, 1.50 mmol), 4-(tert-Butoxycarbonyl)morpholine-2-carboxylic acid (CAS number 189321-66-2, 346.9 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred at ambient temperature. Once the reaction was deemed complete by LCMS, 15.0 ml of 2N HCl was added and the resulting precipitant was filtered. The solid was washed once with 5 ml of 2N HCl and 5 ml of water. The dried solid was milled into a fine powder and was treated with 10 ml of saturated aqueous NaHCO₃ and 5 ml of methylene chloride. This biphasic suspension was shaken vigorously and filtered to provide the title compound dark solid bis-sodium salt (262.4 mg, 84%). LCMS (ESI+): R_{f}=5.91, (M+H)+Na=803.70; HRMS (ESI−): calculated for C₃₈H₃₉N₂O₁₄S m/z [M−H]⁻: 779.2127, observed 779.2143; ¹H NMR 300 MHz DMSO δ: 7.94 (t, J=2.93 Hz, 1H), 7.76 (ddd, J=1.76, 3.08, 6.30 Hz, 1H), 7.54-7.59 (m, 1H), 4.86-4.93 (m, 1H), 3.83-3.89 (m, 2H), 3.76-3.82 (m, 2H), 2.86-2.89 (M, 2H), 1.38 (s, 9H).

### Example 234—Synthesis of 2-(2-methoxyacetyl)-5-[2-(2-methoxyacetyl)-1,3-dioxo-2,3-dihydro-1H-indene-5-carbonyl]-2,3-dihydro-1H-indene-1,3-dione

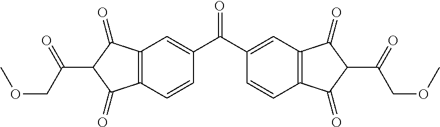

To a solution of dimethylaminopyridine (DMAP, 183 mg, 1.50 mmol), methoxy acetic acid (CAS number 625-45-6, 135.1 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred for 1 hr. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1N NaOH and 5 ml of methylene chloride. This biphasic suspension was shaken vigorously and filtered to provide the title compound dark solid bis-sodium salt (235.4 mg, 93%). LCMS (ESI+): R_{f}=4.16, (M+H)+=463.37; HRMS (ESI−): calculated for C₂₅H₁₈O₉ m/z [M−H]⁻: 461.0878, observed 461.0875; ¹H NMR 300 MHz DMSO δ: 7.98-8.04 (m, 1H), 7.83 (s, 1H), 7.76 (d, J=7.62 Hz, 1H), 4.60-4.63 (m, 1H), 3.29-3.37 (m, 2H), 3.28 (br. s, 3H).

### Example 235—Synthesis of 2-[2-(tert-butoxy)acetyl]-5-{2-[2-(tert-butoxy)acetyl]-1,3-dioxo-2,3-dihydro-1H-indene-5-carbonyl}-2,3-dihydro-1H-indene-1,3-dione

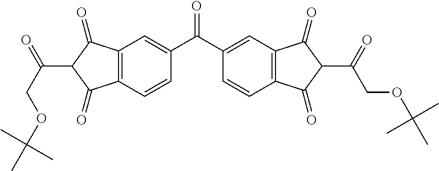 [SMILES: CC(C)(C)OCC(=O)C1C(=O)c2ccc(C(=O)c3ccc4c(c3)C(=O)C(C(=O)COC(C)(C)C)C4=O)cc2C1=O]

To a solution of dimethylaminopyridine (DMAP, 183 mg, 1.50 mmol), tertbutoxy acetic acid (CAS number 13211-32-0, 198.2 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of saturated aqueous NaHCO₃ and 5 ml of methylene chloride. This biphasic suspension was shaken vigorously and filtered to provide the title compound dark solid bis-sodium salt (280.5 mg, 95%). LCMS (ESI+): R_{f}=6.47, (M+H)+=547.45; HRMS (ESI−): calculated for C₃₁H₃₀O₉ m/z [M−H]⁻:545.1817, observed 545.1812; ¹H NMR 300 MHz DMSO δ: 7.86 (dd, J=1.17, 7.62 Hz, 1H), 7.62 (s, 1H), 7.53 (d, J=7.62 Hz, 1H), 4.43-4.49 (m, 2H), 1.11-1.19 (m, 9H).

### Example 236—Synthesis of 2-[(2S)-2-methoxypropanoyl]-5-{2-[(2S)-2-methoxypropanoyl]-1,3-dioxo-2,3-dihydro-1H-indene-5-carbonyl}-2,3-dihydro-1H-indene-1,3-dione

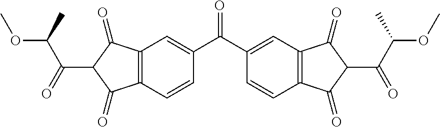

To a solution of dimethylaminopyridine (DMAP, 183 mg, 1.50 mmol), (S)-(−)-2-methoxy propionic acid (CAS number 23953-00-6, 156.2 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15.0 ml of 2N HCl was added and the resulting precipitant was filtered. The solid was washed once with 5 ml of 2N HCl and 5 ml of water. The dried solid was milled into a fine powder and was treated with 10 ml of 1 N NaOH and 5 ml of methylene chloride. This biphasic suspension was shaken vigorously and filtered to provide the title compound dark solid bis-sodium salt (184.4 mg, 69%). LCMS (ESI+): R_{f}=5.27, (M+H)⁺=491.45; HRMS (ESI−): calculated for C₂₇H₂₁O₉ m/z [M−H]⁻: 489.1191, observed 489.1210; ¹H NMR 300 MHz DMSO δ 8.01 (dd, J=1.17, 7.62 Hz, 1H), 7.82 (s, 1H), 7.77 (d, J=7.62 Hz, 1H), 5.04-5.14 (m, 1H), 3.16-3.24 (m, 3H), 1.21-1.29 (m, 3H).

### Example 237—Synthesis of 2-[(2R)-2-methoxypropanoyl]-5-({2-[(2S)-2-methoxypropanoyl]-1,3-dioxo-2,3-dihydro-1H-inden-5-yl}sulfonyl)-2,3-dihydro-1H-indene-1,3-dione

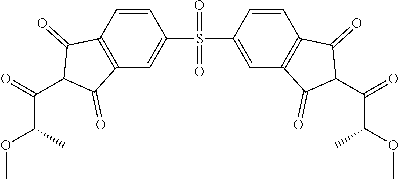 [SMILES: CO[C@@H](C)C(=O)C1C(=O)c2ccc(S(=O)(=O)c3ccc4c(c3)C(=O)C(C(=O)[C@@H](C)OC)C4=O)cc2C1=O]

To a solution of dimethylaminopyridine (DMAP, 183 mg, 1.50 mmol), (S)-(−)-2-methoxy propionic acid (CAS number 23953-00-6, 156.2 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1N NaOH and 5 ml of methylene chloride. This biphasic suspension was shaken vigorously and filtered to provide the title compound dark solid bis-sodium salt (184.4 mg, 69%). LCMS (ESI+): R_{f}=4.72, (M+H)⁺=527.29; HRMS (ESI−): calculated for C₂₆H₂₁O₁₀S m/z [M−H]⁻: 525.0861, observed 525.0879; ¹H NMR 300 MHz DMSO δ 8.09-8.15 (m, 1H), 7.78 (d, J=1.17 Hz, 1H), 7.55-7.62 (m, 1H), 3.11-3.17 (m, 3H), 1.12 (d, J=7.03 Hz, 3H).

### Example 238—Synthesis of 5-{1,3-dioxo-2-[2-(pyridin-3-yl)acetyl]-2,3-dihydro-1H-indene-5-carbonyl}-2-[2-(pyridin-3-yl)acetyl]-2,3-dihydro-1H-indene-1,3-dione

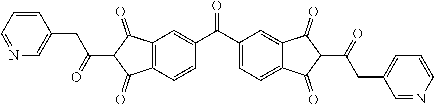 [SMILES: O=C(c1ccc2c(c1)C(=O)C(C(=O)Cc1cccnc1)C2=O)c1ccc2c(c1)C(=O)C(C(=O)Cc1cccnc1)C2=O]

To a solution of dimethylaminopyridine (DMAP, 183 mg, 1.50 mmol), 3-pyridylacetic acid (CAS number 501-81-5, 205.7 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1N NaOH and 10 ml of methylene chloride. This biphasic suspension was shaken vigorously and filtered to provide the title compound dark solid bis-sodium salt (114.1 mg, 38%). LCMS (ESI+): R_{f}=3.27, (M+H)+=557.62; HRMS (ESI−): calculated for C₃₃H₁₉N₂O₇ m/z [M−H]⁻: 555.1198, observed 555.1204; ¹H NMR 300 MHz DMSO δ: 8.41-8.47 (m, 1H), 8.32-8.38 (m, 1H), 7.86-7.92 (m, 1H), 7.55-7.69 (m, 3H), 7.23-7.29 (m, 1H), 4.11-4.18 (m, 2H).

### Example 239 Synthesis of 5-{1,3-dioxo-2-[2-(pyridin-4-yl)acetyl]-2,3-dihydro-1H-indene-5-carbonyl}-2-[2-(pyridin-4-yl)acetyl]-2,3-dihydro-1H-indene-1,3-dione

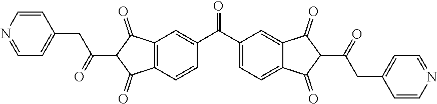 [SMILES: O=C(c1ccc2c(c1)C(=O)C(C(=O)Cc1ccncc1)C2=O)c1ccc2c(c1)C(=O)C(C(=O)Cc1ccncc1)C2=O]

To a solution of dimethylaminopyridine (DMAP, 183 mg, 1.50 mmol), 4-pyridylacetic acid·HCl (CAS number 6622-91-9, 260.4 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1N NaOH and 10 ml of methylene chloride. This biphasic suspension was shaken vigorously and filtered to provide the title compound dark solid bis-sodium salt (126.1 mg, 42%). LCMS (ESI+): R_{f}=2.58, (M+H)+=557.44; HRMS (ESI−): calculated for C₃₃H₁₉N₂O₇ m/z [M−H]⁻: 555.1198, observed 555.11183; ¹H NMR 300 MHz DMSO δ: 8.35-8.44 (m, 1H), 8.05-8.11 (m, 1H), 7.87-7.96 (m, 2H), 7.66-7.70 (m, 1H), 7.56-7.62 (m, 1H) 7.22-7.30 (m, 1H), 4.12-4.22 (m, 2H).

### Example 240—Synthesis of tert-butyl 2-[5-(2-{4-[(tert-butoxy)carbonyl]morpholine-2-carbonyl}-1,3-dioxo-2,3-dihydro-1H-indene-5-carbonyl)-1,3-dioxo-2,3-dihydro-1H-indene-2-carbonyl]morpholine-4-carboxylate

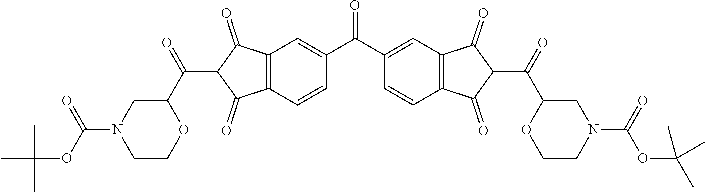 [SMILES: CC(C)(C)OC(=O)N1CCOC(C(=O)C2C(=O)c3ccc(C(=O)c4ccc5c(c4)C(=O)C(C(=O)C4CN(C(=O)OC(C)(C)C)CCO4)C5=O)cc3C2=O)C1]

To a solution of dimethylaminopyridine (DMAP, 183 mg, 1.50 mmol), 4-(tert-butoxycarbonyl)-morpholine-carboxcyclic acid (CAS number 189321-66-2, 205.7 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered to provide the title compound as a brown solid (236.6 mg, 60%). LCMS (ESI+): R_{f}=6.55, (M+H)+Na=767.54; HRMS (ESI−): calculated for C₄₂H₂₃N₂O₁₃ m/z [M−H]⁻: 743.2458, observed 743.2457; ¹H NMR 300 MHz DMSO δ: 7.90-7.96 (m, 1H), 7.72-7.76 (m, 1H), 7.63-7.68 (m, 1H), 4.99-5.07 (m, 1H), 3.82-3.97 (m, 2H), 3.62-3.72 (m, 1H), 3.39-3.52 (m, 1H), 3.14-3.18 (m, 1H), 2.88-3.02 (m, 1H), 2.73-2.80 (m, 1H), 1.40 (s, 9H).

### Example 241—Synthesis of 5-{[1,3-dioxo-2-(2-phenylpyridine-4-carbonyl)-2,3-dihydro-1H-inden-5-yl]sulfonyl}-2-(2-phenylpyridine-4-carbonyl)-2,3-dihydro-1H-indene-1,3-dione

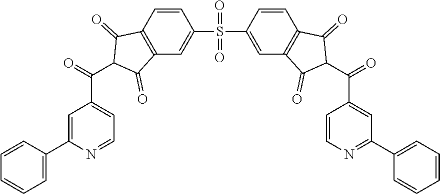 [SMILES: O=C(c1ccnc(-c2ccccc2)c1)C1C(=O)c2ccc(S(=O)(=O)c3ccc4c(c3)C(=O)C(C(=O)c3ccnc(-c5ccccc5)c3)C4=O)cc2C1=O]

To a solution of dimethylaminopyridine (DMAP, 183 mg, 1.50 mmol), 2-phenyl-isonicotinic acid (CAS number 55240-51-2, 298.8 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1N NaOH and 10 ml of methylene chloride. This biphasic suspension was shaken vigorously and filtered to provide the title compound dark solid bis-sodium salt (224.4 mg, 58%). LCMS (ESI+): R_{f}=3.83, (M+H)⁺=717.77; HRMS (ESI−): calculated for C₄₂H₂₄N₂O₈S m/z [M−H]⁻: 715.1180, observed 715.1174; ¹H NMR 300 MHz DMSO δ: 8.63-8.73 (m, 1H), 8.12-8.19 (m, 1H), 7.99-8.08 (M, 1H), 7.87-7.93 (m, H), 7.78-7.84 (m, 1H), 7.58-7.64 (m, 1H), 7.42-7.54 (m, 3H), 7.34-7.40 (m, 1H).

### Example 242—Synthesis of 2-[2-(4-fluorophenyl)propanoyl]-5-{2-[2-(4-fluorophenyl)propanoyl]-1,3-dioxo-2,3-dihydro-1H-indene-5-carbonyl}-2,3-dihydro-1H-indene-1,3-dione

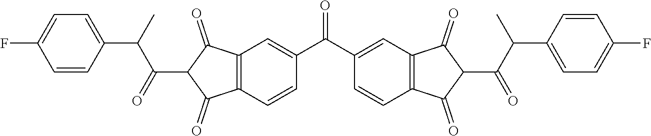 [SMILES: CC(C(=O)C1C(=O)c2ccc(C(=O)c3ccc4c(c3)C(=O)C(C(=O)C(C)c3ccc(F)cc3)C4=O)cc2C1=O)c1ccc(F)cc1]

To a solution of dimethylaminopyridine (DMAP, 183 mg, 1.50 mmol), 2-(4-Fluorophenyl)propanoic acid (CAS number 75908-73-5, 252.3 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4,159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1N NaOH and 5 ml of methylene chloride. This biphasic suspension was shaken vigorously and filtered to provide the title compound dark solid bis-sodium salt (312.8 mg, 94%). LCMS (ESI+): R_{f}=7.94, (M+H)⁺=619.58; HRMS ((ESI−): calculated for C₃₇H₂₄F₂O₇ m/z [M−H]⁻: 617.1417, observed 617.1402; ¹H NMR 300 MHz DMSO δ 7.82-7.88 (m, 1H), 7.61-7.65 (m, 1H), 7.53 (d, J=7.62 Hz, 1H), 7.31-7.40 (m, 2H), 6.97-7.08 (m, 2H), 5.24-5.34 (m, 1H), 1.19-1.31 (m, 3H).

### Example 243—Synthesis of 2-[2-(4-fluorophenyl)propanoyl]-5-({2-[2-(4-fluorophenyl)propanoyl]-1,3-dioxo-2,3-dihydro-1H-inden-5-yl}sulfonyl)-2,3-dihydro-1H-indene-1,3-dione

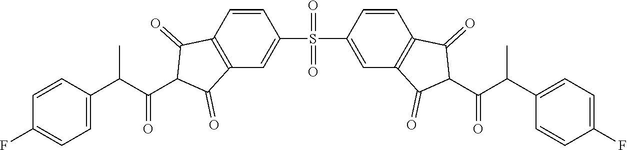 [SMILES: CC(C(=O)C1C(=O)c2ccc(S(=O)(=O)c3ccc4c(c3)C(=O)C(C(=O)C(C)c3ccc(F)cc3)C4=O)cc2C1=O)c1ccc(F)cc1]

To a solution of dimethylaminopyridine (DMAP, 183 mg, 1.50 mmol), 2-(4-Fluorophenyl)propanoic acid (CAS number 75908-73-5, 252.3 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1N NaOH and 5 ml of methylene chloride. This biphasic suspension was shaken vigorously and filtered to provide the title compound dark solid bis-sodium salt (312.8 mg, 94%). LCMS (ESI+): R_{f}=7.61, (M+H)⁺=655.55; HRMS (ESI−): calculated for C₃₆H₂₃F₂O₈S m/z [M−H]⁻: 653.1087, observed 653.1074; ¹H NMR 300 MHz DMSO δ 8.03-8.10 (m, 1H), 7.71-7.75 (m, 1H), 7.51-7.57 (m, 1H), 7.24-7.34 (m, 2H), 6.94-7.04 (m, 2H), 5.16-5.26 (m, 1H), 1.19-1.28 (m, 3H).

### Example 244 Synthesis of 2-bromo-5-(2-bromo-1,3-dioxo-2,3-dihydro-1H-indene-5-carbonyl)-2,3-dihydro-1H-indene-1,3-dione

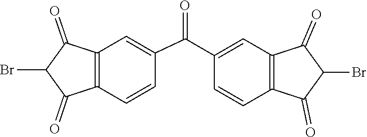 [SMILES: O=C(c1ccc2c(c1)C(=O)C(Br)C2=O)c1ccc2c(c1)C(=O)C(Br)C2=O]

To a 500 ml 3-neck flask equipped with a mechanical stirrer, temperature probe and N₂ balloon, was added 5,5′-carbonylbis(1H-indene-1,3(2H)-dione (Example 4, 2.51 g, 7.92 mmol), SiO₂ (0.52 g, 8.66 mol) and DCE (50 ml) to produce a slurry. To this was added NBS (2.97 g. 16.68 mmol), which briefly turned the mixture a deep orange color. The mixture was allowed to stir at ambient temperature and followed by LCMS. As the reaction progressed it became a very thick orange slurry. Once complete, the reaction was diluted with 50 ml of DCE, then filtered to remove the SiO₂. After filtration, charge 50 ml of 10 wt % sodium metabisulfite solution and transfer to a separatory funnel. The layers were split and the lower organic was dried with sodium sulfite, filtered and concentrated to a foam. The foam was dissolved, concentrated on silica and then purified by column chromatography using EtOAc/hexanes to provide the product as a fine white solid (1.25 g, 35% yield). LCMS (ESI−): R_{f}=7.91, (M−H)⁻=475.13; HRMS (ESI−): calculated for C₁₉H₇FBr₂O₅ m/z [M−H]⁻: 472.8666, observed 472.8648; ¹H NMR 300 MHz CDCl₃ 8.40-8.46 (m, 1H), 8.38 (s, 1H), 8.28-8.34 (m, 1H), 1.49-1.59 (bs 1H).

### Example 245—Synthesis of 4-(5-{[2-(3-carboxypropanoyl)-1,3-dioxo-2,3-dihydro-1H-inden-5-yl]oxy}-1,3-dioxo-2,3-dihydro-1H-inden-2-yl)-4-oxobutanoic acid

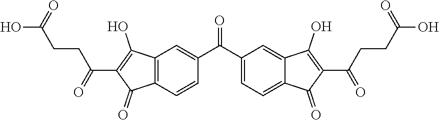 [SMILES: O=C(O)CCC(=O)C1=C(O)c2cc(C(=O)c3ccc4c(c3)C(O)=C(C(=O)CCC(=O)O)C4=O)ccc2C1=O]

To a solution of dimethylaminopyridine (DMAP, 183 mg, 1.50 mmol), monomethyl succinate (CAS number 3878-55-5, 198.2 mg, 1.50 mmol) in anhydrous DMF (10 ml) was added EDCI·HCl (286.5 mg, 1.50 mmol) and the contents were stirred for 10 min. 5-(2,3-dihydro-1,3dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1N NaOH and 5 ml of methylene chloride. This biphasic suspension was shaken vigorously and the lower DCM layer was dropped. The basic aqueous layer was extracted twice with 10 ml of DCM and dropped. The combined DCM washes contained no product and were discarded. The basic aqueous layer was acidified by the addition of excess 2N HCl, resulting in the precipitation of the sodium salt. This suspension was allowed to stir overnight to affect the hydrolysis of the methyl ester and the free acid was then filtered to provide the title compound as rust red solid bis-sodium salt (187.1 mg, 68%). LCMS (ESI+): R_{f}=4.51, (M+H)⁺=507.70; HRMS (ESI−): calculated for C₂₆H₁₇O₁₁ m/z [M−H]⁻: 505.0776, observed 505.0763; ¹H NMR 300 MHz DMSO δ 7.78-7.85 (m, 1H), 7.44-7.50 (m, 1H), 7.31-7.36 (m, 1H), 3.11-3.19 (m, 2H), 2.56 (t, J=7.32 Hz, 2H).

### Example 246—Synthesis of N-[2-(5-{[2-(2-acetamidoacetyl)-1,3-dioxo-2,3-dihydro-1H-inden-5-yl]oxy}-1,3-dioxo-2,3-dihydro-1H-inden-2-yl)-2-oxoethyl]acetamide

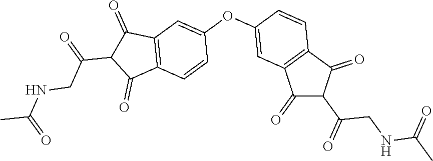 [SMILES: CC(=O)NCC(=O)C1C(=O)c2ccc(Oc3ccc4c(c3)C(=O)C(C(=O)CNC(C)=O)C4=O)cc2C1=O]

To a solution of dimethylaminopyridine (DMAP, 183 mg, 1.50 mmol), N-acetyl-glycine (CAS number 543-24-8, 175.7 mg, 1.50 mmol) in anhydrous DMF (10 ml) was added EDCI·HCl (286.5 mg, 1.50 mmol) and the contents were stirred for 10 min. 5-(2,3-dihydro-1,3dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1N NaOH and 5 ml of methylene chloride. This biphasic suspension was shaken vigorously to provide a thick emulsion, but no precipitate. The emulsion was diluted with additional 10 ml of water and allowed to split. The product rich aqueous was kept, and treated with excess 2N HCl to provide a precipitate which was filtered to provide the title compound as a rust red solid (157.9 mg, 61%). LCMS (ESI−): R_{f}=10.54, (M+H)+=503.33; HRMS (ESI−): calculated for C₂₆H₁₉N₂O₉ m/z [M−H]⁻: 503.1096, observed 503.1087; ¹H NMR 300 MHz DMSO δ: 7.90-7.98 (m, 1H), 7.62-7.67 (m, 1H), 7.28-7.34 (m, 1H), 7.12-7.17 (m, 1H), 4.31-4.42 (m, 2H), 1.83-1.90 (m, 3H).

### Example 247—Synthesis of 2-(2-ethoxyacetyl)-5-{[2-(2-ethoxyacetyl)-1,3-dioxo-2,3-dihydro-1H-inden-5-yl]oxy}-2,3-dihydro-1H-indene-1,3-dione

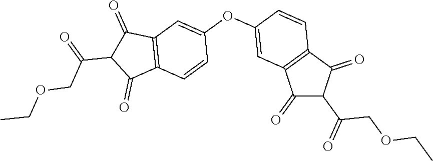 [SMILES: CCOCC(=O)C1C(=O)c2ccc(Oc3ccc4c(c3)C(=O)C(C(=O)COCC)C4=O)cc2C1=O]

To a solution of dimethylaminopyridine (DMAP, 183 mg, 1.50 mmol), 2-(4-Fluorophenyl)propanoic acid (CAS number 75908-73-5, 252.3 mg, 1.50 mmol) in anhydrous DMF (10 ml) was added EDCI·HCl (286.5 mg, 1.50 mmol) and the contents were stirred for min. 5-(2,3-dihydro-1,3dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1N NaOH and 5 ml of methylene chloride. This biphasic suspension was shaken vigorously to provide a thick emulsion, but no precipitate. The emulsion was diluted with additional 10 ml of water and allowed to split. The product rich aqueous was kept, and treated with excess 2N HCl to provide a precipitate which was filtered to provide the title compound as a rust red solid (157.9 mg, 61%). LCMS (ESI−): R_{f}=10.54, (M+H)⁺=503.33; HRMS (ESI−): calculated for C₂₆H₂₁O₉ m/z [M−H]⁻: 477.1191, observed 477.1185; ¹H NMR 300 MHz DMSO δ: 7.71-7.77 (m, 1H), 7.37-7.42 (m, 1H), 7.23 (d, J=2.34 Hz, 1H), 4.7 (s, 2H), 3.47-3.58 (m, 2H) 1.13 (t, J=7.03 Hz, 3H).

### Example 248—Synthesis of 2-[(2S)-2-methoxypropanoyl]-5-({2-[(2S)-2-methoxypropanoyl]-1,3-dioxo-2,3-dihydro-1H-inden-5-yl}oxy)-2,3-dihydro-1H-indene-1,3-dione

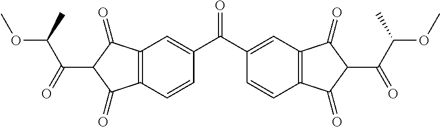

To a solution of dimethylaminopyridine (DMAP, 183 mg, 1.50 mmol), (S)-(−)-2-methoxy propionic acid (CAS number 23953-00-6, 156.2 mg, 1.50 mmol) in anhydrous DMF (10 ml) was added EDCI·HCl (286.5 mg, 1.50 mmol) and the contents were stirred for 10 min. 5-(2,3-dihydro-1,3dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered to provide the title compound (165.1 mg, 69%). LCMS (ESI+): R_{f}=5.80, (M+H)⁺=479.69; HRMS (ESI−): calculated for C₂₆H₂₁O₉ m/z [M−H]⁻: 477.1191, observed 477.1184; ¹H NMR 300 MHz DMSO δ 7.77-7.83 (m, 1H), 7.45 (dd, J=1.76, 8.20 Hz, 1H), 7.26-7.31 (m, 1H), 5.09-5.18 (m, 2H), 3.17-3.26 (m, 3H), 1.24-1.34 (m, 3H).

### Example 249—Synthesis of 2-(2-methoxyacetyl)-5-{[2-(2-methoxyacetyl)-1,3-dioxo-2,3-dihydro-1H-inden-5-yl]oxy}-2,3-dihydro-1H-indene-1,3-dione

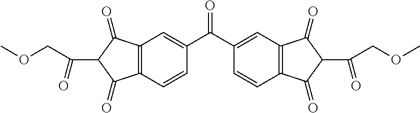

To a solution of dimethylaminopyridine (DMAP, 183 mg, 1.50 mmol), methoxy acetic acid (CAS number 625-45-6, 135.1 mg, 1.50 mmol) in anhydrous DMF (10 ml) was added EDCI·HCl (286.5 mg, 1.50 mmol) and the contents were stirred for 10 min. 5-(2,3-dihydro-1,3dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1N NaOH and 10 ml of methylene chloride. This biphasic suspension was shaken vigorously to provide a thick emulsion, but no precipitate. The emulsion was diluted with additional 10 ml of water and allowed to split. The product rich aqueous was kept, and treated with excess 2N HCl. The acidic aqueous was extracted twice with 10 ml of DCM. The combined organic was dried with sodium sulfate, filtered and concentrated to provide the title compound as a red solid (157.9 mg, 61%). LCMS (ESI+): R_{f}=5.15, (M+H)+=451.67; HRMS (ESI−): calculated for C₂₄H₁₇O₉ m/z [M−H]⁻: 449.0878, observed 449.0881; ¹H NMR 300 MHz DMSO δ: 7.71-7.79 (m, 1H), 7.37-7.44 (m, 1H), 7.23 (d, J=2.34 Hz, 1H), 4.60-4.68 (m, 2H), 3.27-3.38 (m, 3H), 2.05-2.09 (m, 1H).

### Example 250—Synthesis of 5-(2-cyano-1,3-dioxo-2,3-dihydro-1H-indene-5-carbonyl)-1,3-dioxo-2,3-dihydro-1H-indene-2-carbonitrile

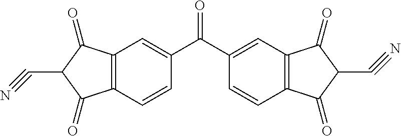 [SMILES: N#CC1C(=O)c2ccc(C(=O)c3ccc4c(c3)C(=O)C(C#N)C4=O)cc2C1=O]

To a suspension of 5,5′-carbonylbis(1H-indene-1,3(2H)-dione (Example 4, 157.4 mg, 0.50 mmol) and Cesium carbonate (484.6 mg, 1.49 mmol) in DCE (5 ml)−water (1 ml) was added tosyl cyanide (222.8 mg, 1.07 mmol). The resulting mixture was allowed to stir at ambient temperature. Once the reaction was deemed complete by LCMS, 15 ml of 2N HCl was charged to the vial. The resulting slurry was allowed to age for NLT 5 min, then was isolated by filtration. The resulting brown solid was dried in a vacuum oven at 45° C. and full house vacuum for 24 hrs, to provide the title compound as a brown solid (149.2 mg, 82% yield). LCMS (ESI+): Rf=3.97 min., (M+H)+=369.66; HRMS (ESI−): calculated for C₂₁H₈N₂O₅ m/z [M−H]⁻: 368.04387, observed 368.0454; ¹H NMR 300 MHz DMSO δ: 7.77-7.82 (m, 1H), 7.55-7.58 (m, 1H), 7.44-7.48 (m, 1H), 3.88 (s, 1H).

### Example 251—Synthesis of 2-cyclopentanecarbonyl-5-[(2-cyclopentanecarbonyl-1,3-dioxo-2,3-dihydro-1H-inden-5-yl)oxy]-2,3-dihydro-1H-indene-1,3-dione

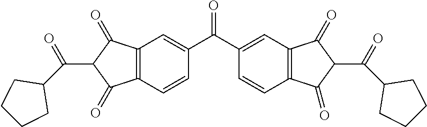 [SMILES: O=C(c1ccc2c(c1)C(=O)C(C(=O)C1CCCC1)C2=O)c1ccc2c(c1)C(=O)C(C(=O)C1CCCC1)C2=O]

To a solution of dimethylaminopyridine (DMAP, 183 mg, 1.50 mmol), cyclopentane carbocyclic acid (CAS number 3400-45-1, 171.2 mg, 1.50 mmol) in anhydrous DMF (10 ml) was added EDCI·HCl (286.5 mg, 1.50 mmol) and the contents were stirred for 10 min. 5-(2,3-dihydro-1,3dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water to provide the title compound as a red solid (196.9 mg, 78%). LCMS (ESI+): R_{f}=8.27, (M+H)⁺=499.73; HRMS (ESI−): calculated for C₃₀H₂₅O₉ m/z [M−H]⁻: 497.1606, observed 497.1602; ¹H NMR 300 MHz DMSO δ: 7.85-7.90 (m, 1H), 7.50-7.56 (m, 1H), 7.38-7.41 (m, 1H), 3.90-4.00 (m, 1H), 1.83-1.98 (m, 2H), 1.59-1.82 (m, 6H).

### Example 252—Synthesis of 2-[4-(2-methylphenyl)-4-oxobutanoyl]-5-{2-[4-(2-methylphenyl)-4-oxobutanoyl]-1,3-dioxo-2,3-dihydro-1H-indene-5-carbonyl}-2,3-dihydro-1H-indene-1,3-dione

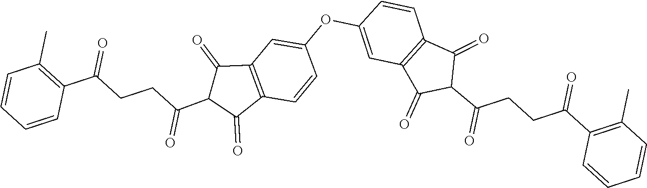 [SMILES: Cc1ccccc1C(=O)CCC(=O)C1C(=O)c2ccc(Oc3ccc4c(c3)C(=O)C(C(=O)CCC(=O)c3ccccc3C)C4=O)cc2C1=O]

To a solution of dimethylaminopyridine (DMAP, 183 mg, 1.50 mmol), 2-Methyl-γ-oxobenzenebutanoic acid (CAS number 6939-36-2, 288.3 mg, 1.50 mmol) in anhydrous DMF (10 ml) was added EDCI·HCl (286.5 mg, 1.50 mmol) and the contents were stirred stirred for 10 min. 5,5′-Carbonylbis(1H-indene-1,3(2H)-dione (Example 4,159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water to provide the title compound as a tan solid (254.3 mg, 76%). LCMS (ESI+): R_{f}=7.47, (M+H)+=667.75; HRMS (ESI−): calculated for C₄₁H₃₀O₉ m/z [M−H]⁻: 665.1817, observed 665.1821; ¹H NMR 300 MHz DMSO δ: 8.00-8.05 (m, 1H), 7.84-7.87 (m, 1H), 7.76-7.83 (m, 2H), 7.37-7.44 (m, 1H), 7.24-7.34 (m, 2H), 3.23 (dd, J=4.10, 7.03 Hz 4H), 2.38 (s, 3H).

### Example 253—Synthesis of 2-[4-(2,3-dichlorophenyl)-4-oxobutanoyl]-5-{2-[4-(2,3-dichlorophenyl)-4-oxobutanoyl]-1,3-dioxo-2,3-dihydro-1H-indene-5-carbonyl}-2,3-dihydro-1H-indene-1,3-dione

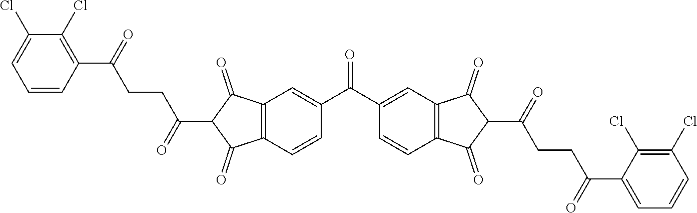 [SMILES: O=C(c1ccc2c(c1)C(=O)C(C(=O)CCC(=O)c1cccc(Cl)c1Cl)C2=O)c1ccc2c(c1)C(=O)C(C(=O)CCC(=O)c1cccc(Cl)c1Cl)C2=O]

To a solution of dimethylaminopyridine (DMAP, 183 mg, 1.50 mmol), 2,3-dichloro-γ-oxobenzenebutanoic acid (CAS number 32003-41-1, 370.6 mg, 1.50 mmol) in anhydrous DMF (10 ml) was added EDCI·HCl (286.5 mg, 1.50 mmol) and the contents were stirred stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was placed in the vacuum oven to dry overnight at 45° C. and full house vacuum. The dried solid was milled into a fine powder and was treated with 10 ml of 1N NaOH and 5 ml of methylene chloride. This biphasic suspension was shaken vigorously and filtered to provide the title compound as the dark solid bis-sodium salt (348.4 mg, 83%). LCMS (ESI+): R_{f}=7.88, (M+H)+=777.65; HRMS (ESI−): calculated for C₃₉H₂₂C₁₄O₉ m/z [M−H]⁻: 772.9945, observed 772.9941; ¹H NMR 300 MHz DMSO δ: 7.82-7.86 (m, 1H), 7.70-7.76 (m, 2H), 7.61-7.64 (m, 1H), 7.53-7.55 (m, 1H), 7.49-7.52 (m, 1H), 3.15-3.21 (m, 2H), 2.97-3.02 (m, 2H).

### Example 254—Synthesis of 2-[4-(2-methylphenyl)-4-oxobutanoyl]-5-({2-[4-(2-methylphenyl)-4-oxobutanoyl]-1,3-dioxo-2,3-dihydro-1H-inden-5-yl}sulfonyl)-2,3-dihydro-1H-indene-1,3-dione

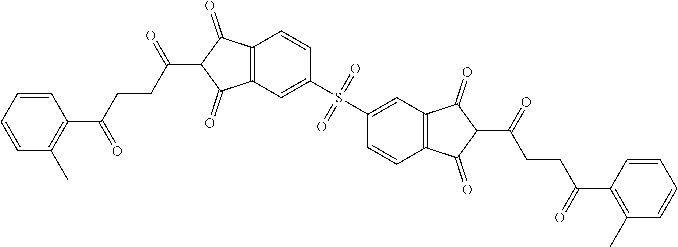 [SMILES: Cc1ccccc1C(=O)CCC(=O)C1C(=O)c2ccc(S(=O)(=O)c3ccc4c(c3)C(=O)C(C(=O)CCC(=O)c3ccccc3C)C4=O)cc2C1=O]

To a solution of dimethylaminopyridine (DMAP, 183 mg, 1.50 mmol), 2-Methyl-γ-oxobenzenebutanoic acid (CAS number 6939-36-2, 288.3 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was placed in the vacuum oven to dry overnight at 45° C. and full house vacuum. The dried solid was milled into a fine powder and was treated with 10 ml of 1N NaOH and 5 ml of methylene chloride. This biphasic suspension was shaken vigorously and filtered to provide the title compound as the dark solid bis-sodium salt (322.6 mg, 85%). LCMS (ESI+): R_{f}=7.02, (M+H)⁺=703.80; HRMS (ESI−): calculated for C₄₀H₃₀O₁₀S m/z [M−H]⁻: 701.1487, observed 701.1480; ¹H NMR 300 MHz DMSO δ: 78.06-8.11 (m, 1H), 7.73-7.81 (m, 2H), 7.52-7.57 (m, 1H), 7.34-7.40 (m, 1H), 7.23-7.32 (m, 2H), 3.06-3.15 (m, 2H), 2.95-3.03 (m, 2H), 2.31-2.38 (m, 3H).

### Example 255—Synthesis of 2-[4-(2,3-dichlorophenyl)-4-oxobutanoyl]-5-({2-[4-(2,3-dichlorophenyl)-4-oxobutanoyl]-1,3-dioxo-2,3-dihydro-1H-inden-5-yl}sulfonyl)-2,3-dihydro-1H-indene-1,3-dione

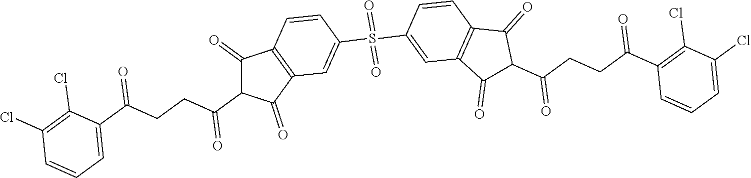 [SMILES: O=C(CCC(=O)C1C(=O)c2ccc(S(=O)(=O)c3ccc4c(c3)C(=O)C(C(=O)CCC(=O)c3cccc(Cl)c3Cl)C4=O)cc2C1=O)c1cccc(Cl)c1Cl]

To a solution of dimethylaminopyridine (DMAP, 183 mg, 1.50 mmol), 2,3-dichloro-γ-oxobenzenebutanoic acid (CAS number 32003-41-1, 370.6 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was placed in the vacuum oven to dry overnight at 45° C. and full house vacuum. The dried solid was milled into a fine powder and was treated with 10 ml of 1N NaOH and 5 ml of methylene chloride. This biphasic suspension was shaken vigorously and filtered to provide the title compound as the dark solid bis-sodium salt (355.5 mg, 84%). LCMS (ESI+): R_{f}=7.35, (M+H)+=813.56; HRMS (ESI−): calculated for C₃₈H₂₂Cl₄O₁₀S m/z [M−H]⁻: 808.9615, observed 808.9616; ¹H NMR 300 MHz DMSO δ: 8.06-8.12 (m, 1H), 7.68-7.77 (m, 3H), 7.56 (d, J=7.62 Hz 1H), 7.42-7.49 (m, 1H), 3.10-3.19 (m, 2H), 2.93-3.01 (m, 2H).

### Example 256—Synthesis of methyl (1S)-2-(5-{2-[(2S)-2-(methoxycarbonyl) cyclohexanecarbonyl]-1,3-dioxo-2,3-dihydro-1H-indene-5-carbonyl}-1,3-dioxo-2,3-dihydro-1H-indene-2-carbonyl)cyclohexane-1-carboxylate

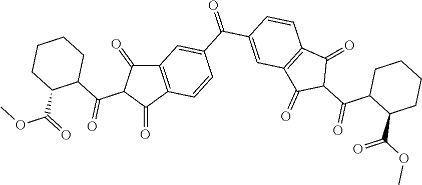 [SMILES: COC(=O)[C@@H]1CCCCC1C(=O)C1C(=O)c2ccc(C(=O)c3ccc4c(c3)C(=O)C(C(=O)C3CCCC[C@H]3C(=O)OC)C4=O)cc2C1=O]

To a solution of dimethylaminopyridine (DMAP, 183 mg, 1.50 mmol), 1,2-Cyclohexanedicarboxylic acid, 1-methyl ester, (1R,2S)-rel- (CAS number 111955-05-6, 279.3 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was placed in the vacuum oven to dry overnight at 45° C. and full house vacuum. The dried solid was milled into a fine powder and was treated with 10 ml of 1N NaOH and 5 ml of methylene chloride. This biphasic suspension was shaken vigorously and filtered to provide the title compound as the dark solid bis-sodium salt (239.4 mg, 60%). LCMS (ESI+): R_{f}=7.62, (M+H)+=655.94; HRMS (ESI−): calculated for C₃₇H₃₄O₁₁ m/z [M−H]⁻: 653.2028, observed 653.2029; ¹H NMR 300 MHz DMSO δ: 7.79-7.90 (m, 1H), 7.57-7.65 (m, 1H), 7.46-7.56 (m, 1H), 3.80-3.94 (m, 1H), 7.49-7.52 (m, 1H), 3.41-3.51 (m, 3H), 1.85-2.08 (m, 2H), 1.44-1.76 (m, 3H), 1.21-1.36 (m, 2H).

### Example 257—Synthesis of methyl (1S,2R)-2-[5-({2-[(1R,2S)-2-(methoxycarbonyl) cyclohexanecarbonyl]-1,3-dioxo-2,3-dihydro-1H-inden-5-yl}sulfonyl)-1,3-dioxo-2,3-dihydro-1H-indene-2-carbonyl]cyclohexane-1-carboxylate

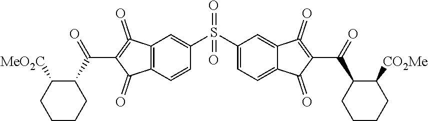

To a solution of dimethylaminopyridine (DMAP, 183 mg, 1.50 mmol), 1,2-Cyclohexanedicarboxylic acid, 1-methyl ester, (1R,2S)-rel-(CAS number 111955-05-6, 279.3 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was placed in the vacuum oven to dry overnight at 45° C. and full house vacuum. The dried solid was milled into a fine powder and was treated with 10 ml of 1N NaOH and 5 ml of methylene chloride. This biphasic suspension was shaken vigorously and filtered to provide the title compound as the dark solid bis-sodium salt (238.8 mg, 65%). LCMS (ESI+): R_{f}=7.36, (M+H)+=691.85; HRMS (ESI−): calculated for C₃₆H₃₄O₁₂S m/z [M−H]⁻: 689.1698, observed 689.1690; ¹H NMR 300 MHz DMSO δ: 8.04-8.14 (m, 1H), 7.68-7.78 (m, 1H), 7.50-7.61 (m, 1H), 3.76-3.91 (m, 1H), 3.40-3.52 (m, 3H), 2.40-2.62 (m, 2H), 1.82-2.12 (m, 2H), 1.42-1.77-(m, 3H), 1.11-1.38 (m, 3H).

### Example 258—Synthesis of 2-[4-(2,3-dichlorophenyl)-4-oxobutanoyl]-5-({2-[4-(2,3-dichlorophenyl)-4-oxobutanoyl]-1,3-dioxo-2,3-dihydro-1H-inden-5-yl}oxy)-2,3-dihydro-1H-indene-1,3-dione

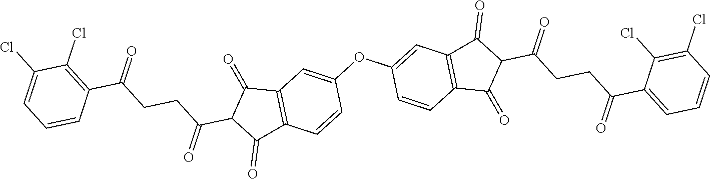 [SMILES: O=C(CCC(=O)C1C(=O)c2ccc(Oc3ccc4c(c3)C(=O)C(C(=O)CCC(=O)c3cccc(Cl)c3Cl)C4=O)cc2C1=O)c1cccc(Cl)c1Cl]

To a solution of dimethylaminopyridine (DMAP, 183 mg, 1.50 mmol),), 2,3-dichloro-γ-oxobenzenebutanoic acid (CAS number 32003-41-1, 370.6 mg, 1.50 mmol) was added EDCI·HCl (286.5 mg, 1.50 mmol) and the contents were stirred for 10 min. 5-(2,3-dihydro-1,3dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water to provide the title compound as a red solid (312.2 mg, 78%). LCMS (ESI+): R_{f}=7.97, (M+H)+=765.63; HRMS (ESI−): calculated for C₃₈H₂₂C₁₄O₉ m/z [M−H]⁻: 760.9945, observed 760.9932; ¹H NMR 300 MHz DMSO δ: 7.69-7.76 (m, 2H), 7.38-7.51 (m, 2H), 7.08-7.14 (m, 1H), 6.98-6.93 (m, 1H), 3.09-3.18 (m, 2H), 2.93-3.02 (m, 2H).

### Example 259—Synthesis of 2-[4-(2-methylphenyl)-4-oxobutanoyl]-5-({2-[4-(2-methylphenyl)-4-oxobutanoyl]-1,3-dioxo-2,3-dihydro-1H-inden-5-yl}oxy)-2,3-dihydro-1H-indene-1,3-dione

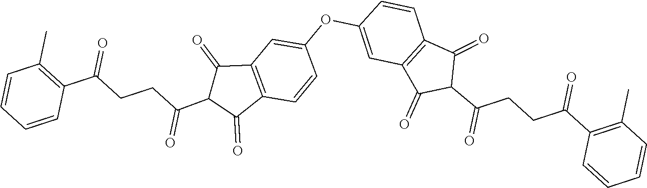 [SMILES: Cc1ccccc1C(=O)CCC(=O)C1C(=O)c2ccc(Oc3ccc4c(c3)C(=O)C(C(=O)CCC(=O)c3ccccc3C)C4=O)cc2C1=O]

To a solution of dimethylaminopyridine (DMAP, 183 mg, 1.50 mmol),), 2-Methyl-γ-oxobenzenebutanoic acid (CAS number 6939-36-2, 288.3 mg, 1.50 mmol) was added EDCI·HCl (286.5 mg, 1.50 mmol) and the contents were stirred for 10 min. 5-(2,3-dihydro-1,3dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water to provide the title compound as a red solid (312.2 mg, 78%). LCMS (ESI+): R_{f}=7.67, (M+H)+=655.80; HRMS (ESI−): calculated for C₄₀H₃₀O₆ m/z [M−H]⁻: 653.1817, observed 653.1815; ¹H NMR 300 MHz DMSO δ: 7.77-7.83 (m, 1H), 7.34-7.44 (m, 2H), 7.23-7.33 (m, 2H), 7.08-7.14 (m, 1H), 6.90-6.94 (m, 1H), 3.06-3.16 (m, 2H), 2.98-3.06 (m, 2H), 2.32-2.39 (M, 3H).

### Example 260—Synthesis of 2-[2-(2,3-difluorophenyl)acetyl]-5-({2-[2-(2,3-difluorophenyl)acetyl]-1,3-dioxo-2,3-dihydro-1H-inden-5-yl}oxy)-2,3-dihydro-1H-indene-1,3-dione

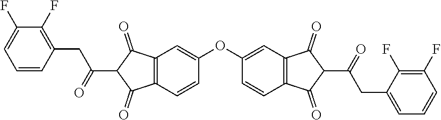 [SMILES: O=C(Cc1cccc(F)c1F)C1C(=O)c2ccc(Oc3ccc4c(c3)C(=O)C(C(=O)Cc3cccc(F)c3F)C4=O)cc2C1=O]

To a solution of dimethylaminopyridine (DMAP, 183 mg, 1.50 mmol),), 2-Methyl-γ-oxobenzenebutanoic acid (CAS number 6939-36-2, 288.3 mg, 1.50 mmol) was added EDCI·HCl (286.5 mg, 1.50 mmol) and the contents were stirred for 10 min. 5-(2,3-dihydro-1,3dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water to provide the title compound as a red solid (312.2 mg, 78%). LCMS (ESI+): R_{f}=7.41, (M+H)+=615.71; HRMS (ESI−): calculated for C₄₀H₃₀O₆ m/z [M−H]⁻: 613.0916, observed 613.0907; ¹H NMR 300 MHz DMSO δ: 7.74-7.80 (m, 1H), 7.40-7.46 (m, 1H), 7.26-7.35 (m, 2H), 7.09-7.19 (m, 2H), 4.30-4.37 (m, 2H).

### Example 261—Synthesis of 5-{1,3-dioxo-2-[2-(2-oxocyclopentyl)acetyl]-2,3-dihydro-1H-indene-5-carbonyl}-2-[2-(2-oxocyclopentyl)acetyl]-2,3-dihydro-1H-indene-1,3-dione

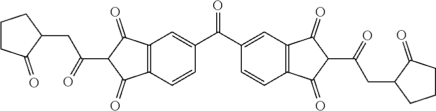 [SMILES: O=C(c1ccc2c(c1)C(=O)C(C(=O)CC1CCCC1=O)C2=O)c1ccc2c(c1)C(=O)C(C(=O)CC1CCCC1=O)C2=O]

To a solution of dimethylaminopyridine (DMAP, 183 mg, 1.50 mmol), 1,2-Cyclohexanedicarboxylic acid, 1-methyl ester, (1R,2S)-rel- (CAS number 111955-05-6, 279.3 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was placed in the vacuum oven to dry overnight at 45° C. and full house vacuum. The dried solid was milled into a fine powder and was treated with 10 ml of 1N NaOH and 5 ml of methylene chloride. This biphasic suspension was shaken vigorously and filtered to provide the title compound as the dark solid bis-sodium salt (239.4 mg, 60%). LCMS (ESI+): R_{f}=7.62, (M+H)+=655.94; HRMS (ESI−): calculated for C₃₇H₃₄O₁₁ m/z [M−H]⁻: 565.1504, observed 565.1503; ¹H NMR 300 MHz DMSO δ: 7.79-7.90 (m, 1H), 7.57-7.65 (m, 1H), 7.46-7.56 (m, 1H), 3.80-3.94 (m, 1H), 7.49-7.52 (m, 1H), 3.41-3.51 (m, 3H), 1.85-2.08 (m, 2H), 1.44-1.76 (m, 3H), 1.21-1.36 (m, 2H).

### Example 262—Synthesis of (1R,2R)-2-({5-[(2-{[(1R,2R)-2-carboxycyclopropyl](hydroxy)methylidene}-1,3-dioxo-2,3-dihydro-1H-inden-5-yl)oxy]-1,3-dioxo-2,3-dihydro-1H-inden-2-ylidene}(hydroxy)methyl)cyclopropane-1-carboxylic acid

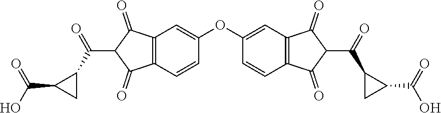 [SMILES: O=C1c2ccc(Oc3ccc4c(c3)C(=O)C(C(=O)[C@@H]3C[C@H]3C(=O)O)C4=O)cc2C(=O)C1C(=O)[C@@H]1C[C@H]1C(=O)O]

To a solution of dimethylaminopyridine (DMAP, 183 mg, 1.50 mmol), 1,2-cyclopropanedicarboxylic acid, 1-methyl ester, (1R,2R)- (CAS number 88335-97-1, 216.2 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was allowed to stir overnight to affect the hydrolysis of the methyl ester. The solid was isolated by filtration, washed once with 5 ml of 2N HCl and 5 ml of water. The solid was dried overnight in the vacuum oven to provide the title compound as a red solid (159.1 mg, 60%). LCMS (ESI+): R_{f}=4.88, M+H=531.60; HRMS (ESI−): calculated for C₂₈H₁₇O₁₁ m/z [M−H]⁻: Exact Mass: 529.0776, observed 529.0784; ¹H NMR 300 MHz DMSO δ: 7.74-7.82 (m, 1H), 7.40-7.49 (m, 1H), 7.29-7.35 (m, 1H), 3.50-3.60 (m, 1H), 2.14-2.24 (m, 1H), 1.44-1.61 (m, 2H).

### Example 263—Synthesis of tert-butyl N-[2-(5-{[2-(2-{[(tert-butoxy)carbonyl](methyl)amino}acetyl)-1,3-dioxo-2,3-dihydro-1H-inden-5-yl]oxy}-1,3-dioxo-2,3-dihydro-1H-inden-2-yl)-2-oxoethyl]-N-methylcarbamate

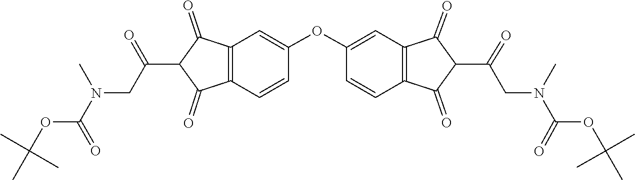 [SMILES: CN(CC(=O)C1C(=O)c2ccc(Oc3ccc4c(c3)C(=O)C(C(=O)CN(C)C(=O)OC(C)(C)C)C4=O)cc2C1=O)C(=O)OC(C)(C)C]

To a solution of dimethylaminopyridine (DMAP, 183 mg, 1.50 mmol), 2-[(tert-Butoxycarbonyl)(methyl)amino]acetic acid (CAS number 13734-36-6, 283.8 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (229.3 mg, 66%). LCMS (ESI+): R_{f}=6.92, M+Na=671.94; HRMS (ESI−): calculated for C₃₄H₃₅N₂O₁₁ m/z [M−H]⁻: Exact Mass: 647.224, observed 647.2256; ¹H NMR 300 MHz DMSO δ: 7.40 (d, J=7.62 Hz, 1H), 7.08-7.14 (m, 1H), 6.89-6.94 (m, 1H), 4.29-4.38 (m, 2H), 2.69-2.79 (m, 3H), 1.25-1.42 (m, 9H).

### Example 264—Synthesis of tert-butyl N-(2-{5-[2-(2-{[(tert-butoxy)carbonyl](methyl)amino}acetyl)-1,3-dioxo-2,3-dihydro-1H-indene-5-carbonyl]-1,3-dioxo-2,3-dihydro-1H-inden-2-yl}-2-oxoethyl)-N-methylcarbamate

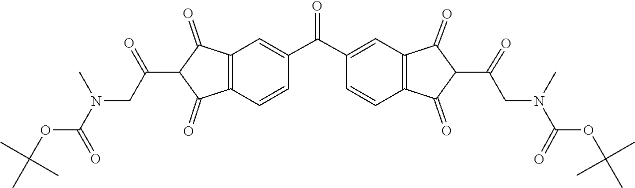 [SMILES: CN(CC(=O)C1C(=O)c2ccc(C(=O)c3ccc4c(c3)C(=O)C(C(=O)CN(C)C(=O)OC(C)(C)C)C4=O)cc2C1=O)C(=O)OC(C)(C)C]

To a solution of dimethylaminopyridine (DMAP, 183 mg, 1.50 mmol), 2-[(tert-Butoxycarbonyl)(methyl)amino]acetic acid (CAS number 13734-36-6, 283.8 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1 N NaOH and 10 ml of methylene chloride. This biphasic suspension was shaken vigorously and filtered to provide the title compound dark solid bis-sodium salt (303.0 mg, 86%). LCMS (ESI+): R_{f}=6.35, M+Na=683.95; HRMS (ESI−): calculated for C₃₅H₃₅N₂O₁₁ m/z [M−H]⁻: Exact Mass: 659.2246, observed 659.2264; ¹H NMR 300 MHz DMSO δ: 7.83-7.89 (m, 11H), 7.61-7.66 (m, 1H), 7.51-7.57 (m, 1H), 4.32-4.40 (m, 2H), 2.69-2.81 (m, 3H), 1.25-1.44 (m, 9H).

### Example 265—Synthesis of N-[4-(5-{[2-(4-acetamidobutanoyl)-1,3-dioxo-2,3-dihydro-1H-inden-5-yl]oxy}-1,3-dioxo-2,3-dihydro-1H-inden-2-yl)-4-oxobutyl]acetamide

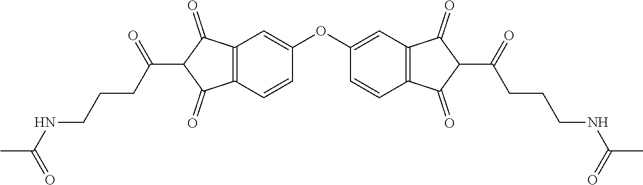 [SMILES: CC(=O)NCCCC(=O)C1C(=O)c2ccc(Oc3ccc4c(c3)C(=O)C(C(=O)CCCNC(C)=O)C4=O)cc2C1=O]

To a solution of dimethylaminopyridine (DMAP, 183 mg, 1.50 mmol), 4-(Acetylamino)butanoic acid (CAS number 3025-96-5, 217.7 mg, 1.50 mmol) in anhydrous DMF (10 ml) was added EDCI·HCl (286.5 mg, 1.50 mmol) and the contents were stirred for min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried to provide title compound (130.0 mg, 46%). LCMS (ESI+): R_{f}=4.48, M+H=561.77; HRMS (ESI−): calculated for C₃₄H₂₁O₉ m/z [M−H]⁻: Exact Mass: 573.1191, observed 573.1207; ¹H NMR 300 MHz DMSO δ: 7.84-7.89 (m, 1H), 7.49-7.54 (m, 1H), 7.36-7.39 (m, 1H), 3.03-3.13 (m, 2H), 2.86-2.94 (m, 2H), 1.67-1.79 (m, 5H).

### Example 266—Synthesis of 2-(1-methylpiperidine-4-carbonyl)-5-[2-(1-methylpiperidine-4-carbonyl)-1,3-dioxo-2,3-dihydro-1H-indene-5-carbonyl]-2,3-dihydro-1H-indene-1,3-dione

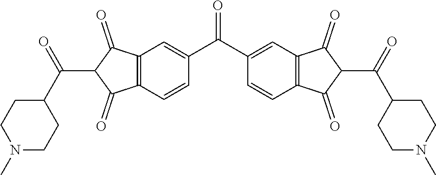 [SMILES: CN1CCC(C(=O)C2C(=O)c3ccc(C(=O)c4ccc5c(c4)C(=O)C(C(=O)C4CCN(C)CC4)C5=O)cc3C2=O)CC1]

To a solution of dimethylaminopyridine (DMAP, 183 mg, 1.50 mmol), 1-Methylpiperidine-4-carboxylic acid hydrochloride (CAS number 71985-80-3, 269.5 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (130.9 mg, 43%). LCMS (ESI+): R_{f}=3.22, M+H=569.94; HRMS (ESI−): calculated for C₃₃H₃₁N₂O₇ m/z [M−H]⁻: Exact Mass: 567.2137, observed 567.2167; ¹H NMR 300 MHz DMSO δ: 7.85 (dd, J=1.46, 7.32 Hz 1H), 7.63 (s, 1H), 7.50-7.55 (m, 1H), 3.83-3.96 (m, 2H), 3.50-3.63 (m, 1H), 5.24-2.68 (m, 2H), 1.81-1.91 (m, 2H), 1.58-1.68 (m, 2H), 1.28-1.42 (m, 9H).

### Example 267—Synthesis of tert-butyl N-[3-(5-{[2-(3-{[(tert-butoxy)carbonyl]amino}propanoyl)-1,3-dioxo-2,3-dihydro-1H-inden-5-yl]oxy}-1,3-dioxo-2,3-dihydro-1H-inden-2-yl)-3-oxopropyl]carbamate

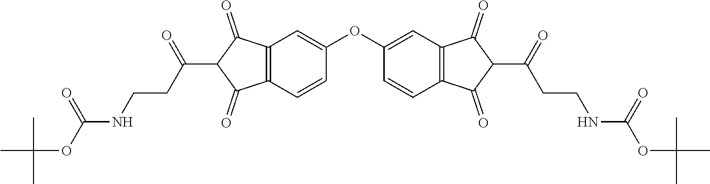 [SMILES: CC(C)(C)OC(=O)NCCC(=O)C1C(=O)c2ccc(Oc3ccc4c(c3)C(=O)C(C(=O)CCNC(=O)OC(C)(C)C)C4=O)cc2C1=O]

To a solution of dimethylaminopyridine (DMAP, 183 mg, 1.50 mmol), N-Boc-R— alanine (CAS number 3303-84-2, 298 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried to provide title compound (253.0 mg, 78%). LCMS (ESI+): R_{f}=5.94, M+H=649.79; HRMS (ESI−): calculated for C₃₄H₃₅N₂O₁₁ m/z [M−H]⁻: Exact Mass: 647.2246, observed 647.2277; ¹H NMR 300 MHz DMSO δ: 7.84-7.92 (m, 1H), 7.48-7.56 (m, 1H), 7.34-7.41 (m, 1H), 6.89-6.99 (m, 1H), 3.18-3.33 (m, 2H), 2.96-3.08 (m, 2H), 1.19-1.40 (m, 9H).

### Example 268—Synthesis of tert-butyl N-(3-{5-[2-(3-{[(tert-butoxy)carbonyl]amino}propanoyl)-1,3-dioxo-2,3-dihydro-1H-indene-5-carbonyl]-1,3-dioxo-2,3-dihydro-1H-inden-2-yl}-3-oxopropyl)carbamate

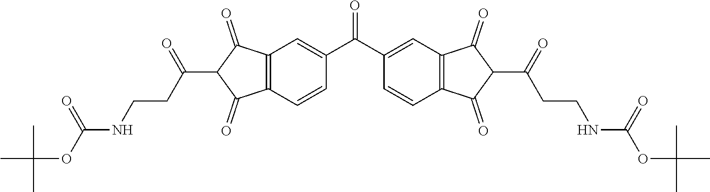 [SMILES: CC(C)(C)OC(=O)NCCC(=O)C1C(=O)c2ccc(C(=O)c3ccc4c(c3)C(=O)C(C(=O)CCNC(=O)OC(C)(C)C)C4=O)cc2C1=O]

To a solution of dimethylaminopyridine (DMAP, 183 mg, 1.50 mmol), N-Boc-R— alanine (CAS number 3303-84-2, 298 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried to provide title compound (77.9 mg, 22%). LCMS (ESI+): R_{f}=5.90, M+H=683.88; HRMS (ESI−): calculated for C₃₅H₃₅N₂O₁₁ m/z [M−H]⁻: Exact Mass: 659.2246, observed 659.2234; ¹H NMR 300 MHz DMSO δ: 8.04-8.12 (m, 1H), 7.84-7.96 (m, 2H), 6.81-6.91 (m, 1H), 3.19-3.31 (m, 2H), 2.97-3.10 (m, 2H), 1.21-1.41 (m, 9H).

### Example 269—Synthesis of N-(4-{5-[2-(4-acetamidobutanoyl)-1,3-dioxo-2,3-dihydro-1H-indene-5-carbonyl]-1,3-dioxo-2,3-dihydro-1H-inden-2-yl}-4-oxobutyl)acetamide

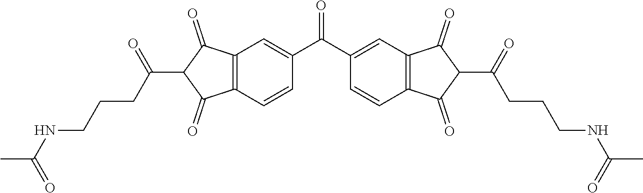 [SMILES: CC(=O)NCCCC(=O)C1C(=O)c2ccc(C(=O)c3ccc4c(c3)C(=O)C(C(=O)CCCNC(C)=O)C4=O)cc2C1=O]

To a solution of dimethylaminopyridine (DMAP, 183 mg, 1.50 mmol), 4-acetamidobutryic acid (CAS number 3025-96-5, 217.7 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried to provide title compound (162.8 mg, 57%). LCMS (ESI+): R_{f}=4.34, M+H=573.79; HRMS (ESI−): calculated for C₃₁H₂₇N₂O₉ m/z [M−H]⁻: Exact Mass: 571.1722, observed 571.1736; ¹H NMR 300 MHz DMSO δ: 8.05-8.11 (m, 1H), 7.84-7.95 (m, 3H), 3.03-3.13 (m, 2H), 2.87-2.96 (m, 2H), 1.67-1.81 (m, 5H).

### Example 270—Synthesis of tert-butyl N-[(2S)-1-[5-({2-[(2S)-2-{[(tert-butoxy) carbonyl]amino}-4-(methylsulfanyl)butanoyl]-1,3-dioxo-2,3-dihydro-1H-inden-5-yl}oxy)-1,3-dioxo-2,3-dihydro-1H-inden-2-yl]-4-(methylsulfanyl)-1-oxobutan-2-yl]carbamate

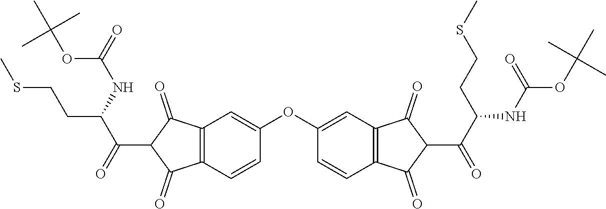 [SMILES: CSCC[C@H](NC(=O)OC(C)(C)C)C(=O)C1C(=O)c2ccc(Oc3ccc4c(c3)C(=O)C(C(=O)[C@H](CCSC)NC(=O)OC(C)(C)C)C4=O)cc2C1=O]

To a solution of dimethylaminopyridine (DMAP, 183 mg, 1.50 mmol), N-(tert-Butoxycarbonyl)methionine (CAS number 2488-15-5, 374.0 mg, 1.50 mmol) in anhydrous DMF (10 ml) was added EDCI·HCl (286.5 mg, 1.50 mmol) and the contents were stirred for min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (217.8 mg, 55%). LCMS (ESI+): R_{f}=6.87, M+H=769.88; HRMS (ESI−): calculated for C₃₈H₄₃N₂O₁₁S₂ m/z [M−H]⁻: Exact Mass: 767.2314, observed 767.2329; ¹H NMR 300 MHz DMSO δ: 7.35-7.46 (m, 1H), 7.07-7.16 (m, 1H), 6.89-6.99 (m, 1H), 6.24-6.36 (m, 1H), 5.00-5.12 (m, 1H), 1.81-2.05 (m, 5H) 1.52-1.68 (m, 2H), 1.26-1.43 (s, 9H).

### Example 271—Synthesis of tert-butyl N-[(2S)-1-(5-{2-[(2S)-2-{[(tert-butoxy)carbonyl]amino}-4-(methylsulfanyl)butanoyl]-1,3-dioxo-2,3-dihydro-1H-indene-5-carbonyl}-1,3-dioxo-2,3-dihydro-1H-inden-2-yl)-4-(methylsulfanyl)-1-oxobutan-2-yl]carbamate

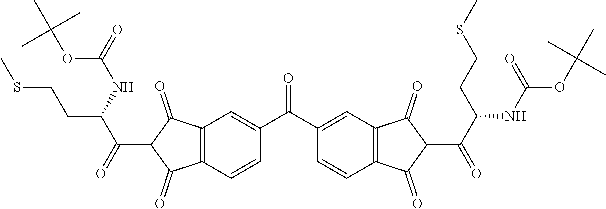 [SMILES: CSCC[C@H](NC(=O)OC(C)(C)C)C(=O)C1C(=O)c2ccc(C(=O)c3ccc4c(c3)C(=O)C(C(=O)[C@H](CCSC)NC(=O)OC(C)(C)C)C4=O)cc2C1=O]

To a solution of dimethylaminopyridine (DMAP, 183 mg, 1.50 mmol),), N-(tert-Butoxycarbonyl)methionine (CAS number 2488-15-5, 374.0 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4,159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1 N NaOH and 10 ml of methylene chloride. This biphasic suspension was shaken vigorously and filtered to provide the title compound dark solid bis-sodium salt (130.9 mg, 43%). LCMS (ESI+): R_{f}=7.38, M+H=781.97; HRMS (ESI−): calculated for C₃₉H₄₃N₂O₁₁S₂ m/z [M−H]⁻: Exact Mass: 779.2314, observed 779.2312; ¹H NMR 300 MHz DMSO δ: 7.83-7.89 (m, 1H), 7.66 (s, 1H), 7.53-7.59 (m, 1H), 6.31-6.38 (m, 1H), 5.04-5.14 (m, 1H), 1.84-2.03 (m, 5H), 1.52-1.68 (m, 2H), 1.29-1.40 (s, 9H).

### Example 272—Synthesis of tert-butyl N-[2-(5-{[2-(2-{[(tert-butoxy)carbonyl]amino}acetyl)-1,3-dioxo-2,3-dihydro-1H-inden-5-yl]oxy}-1,3-dioxo-2,3-dihydro-1H-inden-2-yl)-2-oxoethyl]carbamate

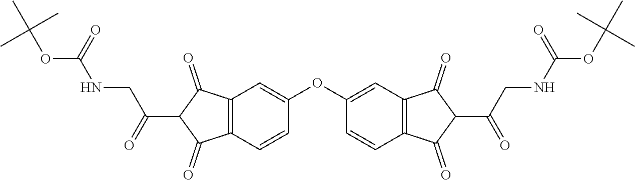 [SMILES: CC(C)(C)OC(=O)NCC(=O)C1C(=O)c2ccc(Oc3ccc4c(c3)C(=O)C(C(=O)CNC(=O)OC(C)(C)C)C4=O)cc2C1=O]

To a solution of dimethylaminopyridine (DMAP, 183 mg, 1.50 mmol), N-(tert-Butoxycarbonyl)glycine (CAS number 4350-20-5, 262.8 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (227.3 mg, 68%). LCMS (ESI+): R_{f}=5.83, peak failed to ionize; HRMS (ESI−): calculated for C₃₂H₃₁N₂O₁₁ m/z [M−H]⁻: Exact Mass: 619.1933, observed 619.1957; ¹H NMR 300 MHz DMSO δ: 7.37-7.44 (m, 1H), 7.07-7.14 (m, 1H), 6.90-6.94 (m, 1H), 6.17-6.26 (m, 1H), 4.08-4.17 (m, 2H), 1.29-1.44 (s, 9H).

### Example 273—Synthesis of tert-butyl (2S)-2-[5-({2-[(2S)-1-[(tert-butoxy)carbonyl]azetidine-2-carbonyl]-1,3-dioxo-2,3-dihydro-1H-inden-5-yl}oxy)-1,3-dioxo-2,3-dihydro-1H-indene-2-carbonyl]azetidine-1-carboxylate

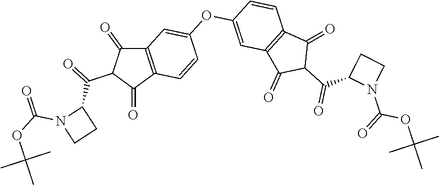 [SMILES: CC(C)(C)OC(=O)N1CC[C@H]1C(=O)C1C(=O)c2ccc(Oc3ccc4c(c3)C(=O)C(C(=O)[C@@H]3CCN3C(=O)OC(C)(C)C)C4=O)cc2C1=O]

To a solution of dimethylaminopyridine (DMAP, 183 mg, 1.50 mmol), (2S)-1-[(tert-Butoxy)carbonyl]azetidine-2-carboxylic acid (CAS number 51077-14-6, 301.8 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (160.0 mg, 45%). LCMS (ESI+): R_{f}=5.83, peak failed to ionize; HRMS (ESI−): calculated for C₃₂H₃₁N₂O₁₁ m/z [M−H]⁻: Exact Mass: 619.1933, observed 619.1957; ¹H NMR 300 MHz DMSO δ: 7.37-7.44 (m, 1H), 7.07-7.14 (m, 1H), 6.90-6.94 (m, 1H), 6.17-6.26 (m, 1H), 4.08-4.17 (m, 2H), 1.29-1.44 (m, 11H).

### Example 274—Synthesis of tert-butyl (2R)-2-[5-({2-[(2R)-1-[(tert-butoxy)carbonyl]piperidine-2-carbonyl]-1,3-dioxo-2,3-dihydro-1H-inden-5-yl}oxy)-1,3-dioxo-2,3-dihydro-1H-indene-2-carbonyl]piperidine-1-carboxylate

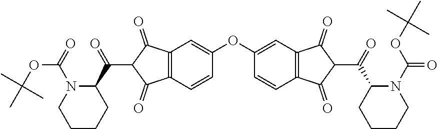 [SMILES: CC(C)(C)OC(=O)N1CCCC[C@@H]1C(=O)C1C(=O)c2ccc(Oc3ccc4c(c3)C(=O)C(C(=O)[C@H]3CCCCN3C(=O)OC(C)(C)C)C4=O)cc2C1=O]

To a solution of dimethylaminopyridine (DMAP, 183 mg, 1.50 mmol), (2R)-1-[(tert-Butoxy)carbonyl]piperidine-2-carboxylic acid (CAS number 28697-17-8, 343.9 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (259.8 mg, 67%). LCMS (ESI+): R_{f}=7.89, M+Na=752.00; HRMS (ESI−): calculated for C₄₀H₄₃N₂O₁₁ m/z [M−H]⁻: Exact Mass: 727.2872, observed 727.2880; ¹H NMR 300 MHz DMSO δ: 7.37-7.40 (m, 1H), 7.05-7.11 (m, 1H), 6.87-6.91 (m, 1H), 5.46-5.64 (m, 1H), 3.74-3.84 (m, 1H), 1.48-1.65 (m, 2H), 1.28-1.46 (m, 5H), 1.16-1.28 (m, 9H).

### Example 275—tert-butyl (2R)-2-(5-{2-[(2R)-1-[(tert-butoxy)carbonyl]piperidine-2-carbonyl]-1,3-dioxo-2,3-dihydro-1H-indene-5-carbonyl}-1,3-dioxo-2,3-dihydro-1H-indene-2-carbonyl)piperidine-1-carboxylate

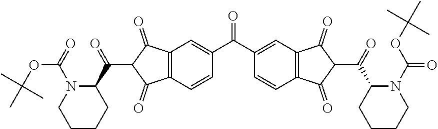 [SMILES: CC(C)(C)OC(=O)N1CCCC[C@@H]1C(=O)C1C(=O)c2ccc(C(=O)c3ccc4c(c3)C(=O)C(C(=O)[C@H]3CCCCN3C(=O)OC(C)(C)C)C4=O)cc2C1=O]

To a solution of dimethylaminopyridine (DMAP, 183 mg, 1.50 mmol), (2R)-1-[(tert-Butoxy)carbonyl]piperidine-2-carboxylic acid (CAS number 28697-17-8, 343.9 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (357.1 mg, 91%). LCMS (ESI+): R_{f}=7.82, peak failed to ionize; HRMS (ESI−): calculated for C₄₁H₄₃N₂O₁₁ m/z [M−H]⁻: Exact Mass: 739.2872, observed 739.2871; ¹H NMR 300 MHz DMSO δ: 7.84 (d, J=7.62 Hz, 1H), 7.60-7.64 (m, 1H), 7.48-7.54 (m, 1H), 5.49-5.65 (m, 1H), 3.74-3.86 (m, 1H), 1.49-1.69 (m, 2H), 1.1.30-1.49 (m, 5H), 1.13-1.28 (m, 9H).

### Example 276—Synthesis of 2-[3-(methylsulfanyl)propanoyl]-5-({2-[3-(methylsulfanyl) propanoyl]-1,3-dioxo-2,3-dihydro-1H-inden-5-yl}oxy)-2,3-dihydro-1H-indene-1,3-dione

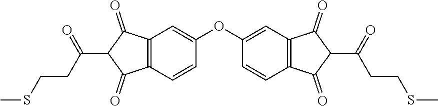

To a solution of dimethylaminopyridine (DMAP, 183 mg, 1.50 mmol), 3-Methylsulfanylpropanoic acid (CAS number 646-01-5, 180.3 mg, 1.50 mmol) in anhydrous DMF (10 ml) was added EDCI·HCl (286.5 mg, 1.50 mmol) and the contents were stirred for min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (144.2 mg, 52%). LCMS (ESI+): R_{f}=6.78, M+H=511.55; HRMS (ESI−): calculated for C₂₆H₂₁O₇S₂ m/z [M−H]⁻: Exact Mass: 509.0734, observed 509.0734; ¹H NMR 300 MHz DMSO δ: 7.27-7.33 (m, 1H), 6.97-7.03 (m, 1H), 6.79-6.83 (m, 1H), 2.83-2.93 (m, 2H), 2.49-2.57 (m, 2H), 1.92-1.98 (m, 3H).

### Example 277—Synthesis of 2-[3-(methylsulfanyl)propanoyl]-5-{2-[3-(methylsulfanyl) propanoyl]-1,3-dioxo-2,3-dihydro-1H-indene-5-carbonyl}-2,3-dihydro-1H-indene-1,3-dione

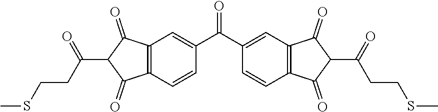

To a solution of dimethylaminopyridine (DMAP, 183 mg, 1.50 mmol), 3-Methylsulfanylpropanoic acid (CAS number 646-01-5, 180.3 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1 N NaOH and 10 ml of methylene chloride. This biphasic suspension was shaken vigorously and filtered to provide the title compound dark solid bis-sodium salt (221.5 mg, 78%). LCMS (ESI+): R_{f}=6.60, M+H=523.56; HRMS (ESI−): calculated for C₂₇H₂₁O₇S₂ m/z [M−H]⁻: Exact Mass: 521.0734, observed 521.0721; ¹H NMR 300 MHz DMSO δ: 7.74-7.81 (m, 1H), 7.55 (s, 1H), 7.43-7.48 (m, 1H), 2.89-3.00 (m, 2H), 2.52-2.61 (m, 2H), 1.96-2.01 (m, 3H).

### Example 278—Synthesis of 2-[3-(methylsulfanyl)propanoyl]-5-({2-[3-(methylsulfanyl) propanoyl]-1,3-dioxo-2,3-dihydro-1H-inden-5-yl}sulfonyl)-2,3-dihydro-1H-indene-1,3-dione

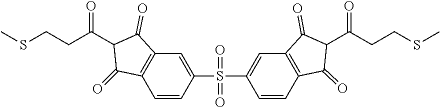

To a solution of dimethylaminopyridine (DMAP, 183 mg, 1.50 mmol), 3-Methylsulfanylpropanoic acid (CAS number 646-01-5, 180.3 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound dark solid bis-sodium salt (220.9 mg, 73%). LCMS (ESI+): R_{f}=6.18, M+H=559.54; HRMS (ESI−): calculated for C₂₆H₂₁O₈S₃ m/z [M−H]⁻: 557.0404, observed 557.0402; ¹H NMR 300 MHz DMSO δ: 7.96-8.03 (m, 1H), 7.63-7.67 (m, 1H), 7.43-7.49 (m, 1H), 2.82-2.93 (m, 2H), 2.47-2.57 (m, 2H), 1.91-1.99 (s, 3H).

### Example 279—Synthesis of tert-butyl N-[(2S,3S)-1-[5-({2-[(2S,3S)-2-{[(tert-butoxy)carbonyl]amino}-3-methylpentanoyl]-1,3-dioxo-2,3-dihydro-1H-inden-5-yl}sulfonyl)-1,3-dioxo-2,3-dihydro-1H-inden-2-yl]-3-methyl-1-oxopentan-2-yl]carbamate

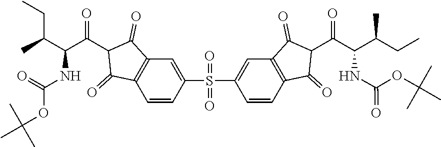 [SMILES: CC[C@H](C)[C@H](NC(=O)OC(C)(C)C)C(=O)C1C(=O)c2ccc(S(=O)(=O)c3ccc4c(c3)C(=O)C(C(=O)[C@@H](NC(=O)OC(C)(C)C)[C@@H](C)CC)C4=O)cc2C1=O]

To a solution of dimethylaminopyridine (DMAP, 183 mg, 1.50 mmol), (2S,3S)-2-(tert-Butoxycarbonylamino)-3-methylpentanoic acid (CAS number 13139-16-7, 346.9 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound dark solid bis-sodium salt (254.6 mg, 62%). LCMS (ESI+): R_{f}=7.33, M+Na=804.99; HRMS (ESI−): calculated for C₄₀H₄₇N₂O₁₂S m/z [M−H]⁻: 779.2855, observed 779.2868; ¹H NMR 300 MHz DMSO δ: 7.94-8.02 (m, 2H), 7.65-7.69 (m, 1H), 7.43-7.50 (m, 1H), 6.43-6.49 (m, 1H), 1.23 (s, 11H), 0.71-0.79 (m, 3H), 0.54-0.63 (m, 3H).

### Example 280—Synthesis of 2-[2-(methylsulfanyl)acetyl]-5-({2-[2-(methylsulfanyl)acetyl]-1,3-dioxo-2,3-dihydro-1H-inden-5-yl}oxy)-2,3-dihydro-1H-indene-1,3-dione

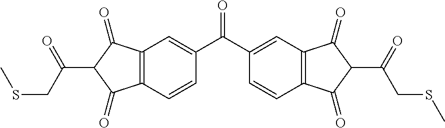

To a solution of dimethylaminopyridine (DMAP, 183 mg, 1.50 mmol), (Methylthio)acetic acid (CAS number 2444-37-3, 159.21 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (111.3 mg, 42%). LCMS (ESI+): R_{f}=6.58, M+H=483.53; HRMS (ESI−): calculated for C₂₄H₁₇O₇S₂ m/z [M−H]⁻: Exact Mass: 481.0421, observed 481.0421; ¹H NMR 300 MHz DMSO δ: 7.51-7.58 (m, 1H), 7.22-7.28 (m, 1H), 7.02-7.08 (m, 1H), 3.75-3.83 (m, 2H), 2.14-2.20 (m, 3H).

### Example 281—Synthesis of 2-[2-(methylsulfanyl)acetyl]-5-{2-[2-(methylsulfanyl)acetyl]-1,3-dioxo-2,3-dihydro-1H-indene-5-carbonyl}-2,3-dihydro-1H-indene-1,3-dione

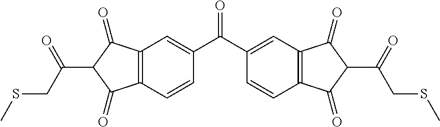

To a solution of dimethylaminopyridine (DMAP, 183 mg, 1.50 mmol), (Methylthio)acetic acid (CAS number 2444-37-3, 159.2 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4,159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (167.1 mg, 62%). LCMS (ESI+): R_{f}=5.53, M+H=495.62; HRMS (ESI−): calculated for C₂₅H₁₇O₇S₂ m/z [M−H]⁻: Exact Mass: 493.0421, observed 493.0403; ¹H NMR 300 MHz DMSO δ: 7.85-7.94 (m, 1H), 7.69-7.75 (m, 1H), 7.61-7.69 (m, 1H), 3.63-3.76 (m, 2H), 1.93-2.03 (m, 3H).

### Example 282—Synthesis of 2-[2-(methylsulfanyl)acetyl]-5-({2-[2-(methylsulfanyl)acetyl]-1,3-dioxo-2,3-dihydro-1H-inden-5-yl}sulfonyl)-2,3-dihydro-1H-indene-1,3-dione

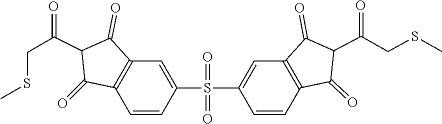

To a solution of dimethylaminopyridine (DMAP, 183 mg, 1.50 mmol), (Methylthio)acetic acid (CAS number 2444-37-3, 159.2 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1 N NaOH and 10 ml of methylene chloride. This biphasic suspension was shaken vigorously and filtered to provide the title compound dark solid bis-sodium salt (173.3 mg, 65%). LCMS (ESI+): R_{f}=5.58, M+H=531.60; HRMS (ESI−): calculated for C₂₄H₁₇O₈S₃ m/z [M−H]⁻: Exact Mass: 529.0091, observed 529.0113; ¹H NMR 300 MHz DMSO δ: 8.28-8.32 (m, 1H), 7.96-7.98 (m, 1H), 7.74-7.80 (m, 1H), 3.82-3.86 (m, 2H), 2.19-2.24 (m, 3H).

### Example 283—2-[4-(methylsulfanyl)butanoyl]-5-({2-[4-(methylsulfanyl)butanoyl]-1,3-dioxo-2,3-dihydro-1H-inden-5-yl}oxy)-2,3-dihydro-1H-indene-1,3-dione

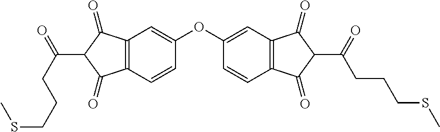

To a solution of dimethylaminopyridine (DMAP, 183 mg, 1.50 mmol), 4-(Methylthio)butanoic acid (CAS number 32391-97-2 201.3 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (125.1 mg, 43%). LCMS (ESI+): R_{f}=6.84, M+H=539.56; HRMS (ESI−): calculated for C₂₈H₂₅O₇S₂ m/z [M−H]⁻: Exact Mass: 537.1047, observed 537.1050; ¹H NMR 300 MHz DMSO δ: 7.23-7.31 (m, 1H), 6.94-7.01 (m, 1H), 6.75-6.81 (m, 1H), 2.62-2.74 (m, 2H), 2.25-2.45 (m, 2H), 1.85-1.95 (m, 3H), 1.56-1.71 (m, 2H).

### Example 284—Synthesis of 2-[4-(methylsulfanyl)butanoyl]-5-{2-[4-(methylsulfanyl) butanoyl]-1,3-dioxo-2,3-dihydro-1H-indene-5-carbonyl}-2,3-dihydro-1H-indene-1,3-dione

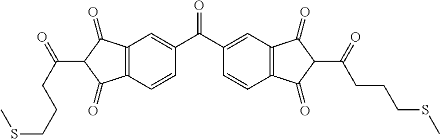

To a solution of dimethylaminopyridine (DMAP, 183 mg, 1.50 mmol), 4-(Methylthio)butanoic acid (CAS number 32391-97-2 201.3 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (164.6 mg, 55%). LCMS (ESI+): R_{f}=6.63, M+H=551.51; HRMS (ESI−): calculated for C₂₉H₂₅O₇S₂ m/z [M−H]⁻: Exact Mass: 549.1047, observed 549.1050; ¹H NMR 300 MHz DMSO δ: 7.72-7.78 (m, 1H), 7.50-7.54 (m, 1H), 7.40-7.46 (m, 1H), 2.67-2.76 (m, 2H), 2.26-2.46 (m, 2H), 1.89-1.96 (m, 3H), 1.58-1.72 (m, 2H).

### Example 285—Synthesis of 2-[4-(methylsulfanyl)butanoyl]-5-({2-[4-(methylsulfanyl) butanoyl]-1,3-dioxo-2,3-dihydro-1H-inden-5-yl}sulfonyl)-2,3-dihydro-1H-indene-1,3-dione

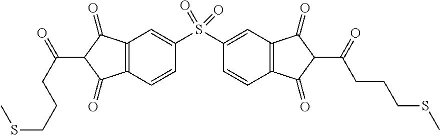

To a solution of dimethylaminopyridine (DMAP, 183 mg, 1.50 mmol), 4-(Methylthio)butanoic acid (CAS number 32391-97-2 201.3 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound dark solid bis-sodium salt (173.9 mg, 55%). LCMS (ESI+): R_{f}=6.33, M+H=587.56; HRMS (ESI−): calculated for C₂₈H₂₅O₈S₃ m/z [M−H]⁻: 585.0717, observed 585.0713; ¹H NMR 300 MHz DMSO δ: 7.90-7.98 (m, 1H), 7.56-7.62 (m, 1H), 7.36-7.43 (m, 1H), 2.56-2.68 (m, 2H), 2.26-2.36 (m, 2H), 1.84-1.90 (m, 3H), 1.53-1.63 (m, 2H).

### Example 286—Synthesis of 2-cyclohexanecarbonyl-5-[(2-cyclohexanecarbonyl-1,3-dioxo-2,3-dihydro-1H-inden-5-yl)oxy]-2,3-dihydro-1H-indene-1,3-dione

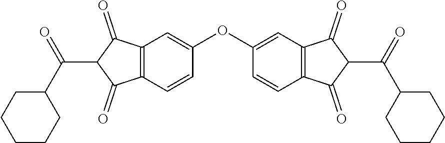 [SMILES: O=C1c2ccc(Oc3ccc4c(c3)C(=O)C(C(=O)C3CCCCC3)C4=O)cc2C(=O)C1C(=O)C1CCCCC1]

To a solution of dimethylaminopyridine (DMAP, 183 mg, 1.50 mmol), Cyclohexylcarboxylic acid (CAS number 98-89-5 192.3 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 153 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (266.7 mg, 92%). LCMS (ESI+): R_{f}=8.31, M+H=539.76; HRMS (ESI−): calculated for C₃₃H₂₉O₇ m/z [M−H]⁻: Exact Mass: 525.1919, observed 525.1919; ¹H NMR 300 MHz DMSO δ: 7.23-7.30 (m, 1H), 6.95-7.01 (m, 1H), 6.75-6.8 (m, 1H), 3.34-3.48 (m, 1H), 1.47-1.66 (m, 6H), 1.08-1.25 (m, 4H).

### Example 287—Synthesis of 2-cyclohexanecarbonyl-5-(2-cyclohexanecarbonyl-1,3-dioxo-2,3-dihydro-1H-indene-5-carbonyl)-2,3-dihydro-1H-indene-1,3-dione

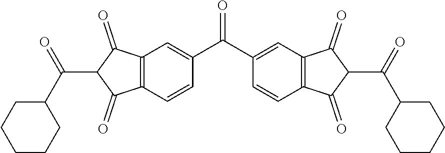 [SMILES: O=C(c1ccc2c(c1)C(=O)C(C(=O)C1CCCCC1)C2=O)c1ccc2c(c1)C(=O)C(C(=O)C1CCCCC1)C2=O]

To a solution of dimethylaminopyridine (DMAP, 183 mg, 1.50 mmol), Cyclohexylcarboxylic acid (CAS number 98-89-5 192.3 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (338.5 mg, 91%). LCMS (ESI+): R_{f}=8.31, M+H=539.76; HRMS (ESI−): calculated for C₃₃H₂₉O₇ m/z [M−H]⁻: Exact Mass: 537.1919, observed 537.1936; ¹H NMR 300 MHz DMSO δ: 7.69-7.76 (m, 1H), 7.48-7.51 (m, 1H), 7.36-7.41 (m, 1H), 3.33-3.49 (m, 1H), 1.48-1.65 (m, 6H), 1.09-1.23 (m, 4H).

### Example 288—Synthesis of 2-cyclohexanecarbonyl-5-[(2-cyclohexanecarbonyl-1,3-dioxo-2,3-dihydro-1H-inden-5-yl)sulfonyl]-2,3-dihydro-1H-indene-1,3-dione

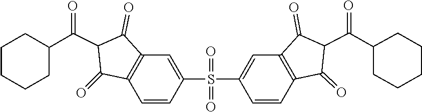 [SMILES: O=C1c2ccc(S(=O)(=O)c3ccc4c(c3)C(=O)C(C(=O)C3CCCCC3)C4=O)cc2C(=O)C1C(=O)C1CCCCC1]

To a solution of dimethylaminopyridine (DMAP, 183 mg, 1.50 mmol), Cyclohexylcarboxylic acid (CAS number 98-89-5 192.3 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound dark solid bis-sodium salt (217.2 mg, 70%). LCMS (ESI+): R_{f}=7.95, M+H=575.81; HRMS (ESI−): calculated for C₃₂H₂₉O₈S m/z [M−H]⁻: 573.1589, observed 573.1585; ¹H NMR 300 MHz DMSO δ: 7.93-8.03 (m, 1H), 7.58-7.65 (m, 1H), 7.39-7.46 (m, 1H), 6.43-6.52 (m, 1H), 1.42-1.67 (m, 6H), 0.93-1.22 (m, 4H).

### Example 289—Synthesis of 5-{1,3-dioxo-2-[2-(pyridin-4-ylsulfanyl)acetyl]-2,3-dihydro-1H-indene-5-carbonyl}-2-[2-(pyridin-4-ylsulfanyl)acetyl]-2,3-dihydro-1H-indene-1,3-dione

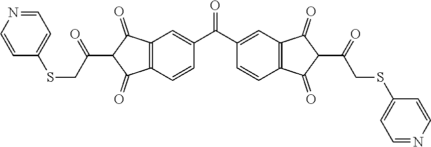 [SMILES: O=C(c1ccc2c(c1)C(=O)C(C(=O)CSc1ccncc1)C2=O)c1ccc2c(c1)C(=O)C(C(=O)CSc1ccncc1)C2=O]

To a solution of dimethylaminopyridine (DMAP, 183 mg, 1.50 mmol), 4-Pyridinylthioacetic acid (CAS number 10351-19-6 253.8 mg, 1.50 mmol) in anhydrous DMF (10 ml) was added EDCI·HCl (286.5 mg, 1.50 mmol) and the contents were stirred for 10 min. 5,5′-Carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound dark solid bis-sodium salt (338.5 mg, 91%). LCMS (ESI+): R_{f}=7.40, peak failed to ionize; HRMS (ESI−): calculated for C₃₃H₂₀N₂O₇S₂ m/z [M−H]⁻: Exact Mass: 619.0639, observed 619.0121; ¹H NMR 300 MHz DMSO δ: 8.20-8.25 (m, 2H), 7.77-7.84 (m, 1H), 7.58-7.61 (m, 1H), 7.47-7.53 (m, 1H), 7.18-7.24 (m, 2H), 4.25-4.32 (m, 2H).

### Example 290—Synthesis of 5-({1,3-dioxo-2-[2-(pyridin-4-ylsulfanyl)acetyl]-2,3-dihydro-1H-inden-5-yl}sulfonyl)-2-[2-(pyridin-4-ylsulfanyl)acetyl]-2,3-dihydro-1H-indene-1,3-dione

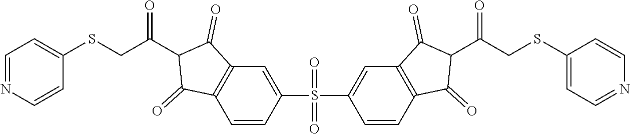 [SMILES: O=C(CSc1ccncc1)C1C(=O)c2ccc(S(=O)(=O)c3ccc4c(c3)C(=O)C(C(=O)CSc3ccncc3)C4=O)cc2C1=O]

To a solution of dimethylaminopyridine (DMAP, 183 mg, 1.50 mmol), 4-Pyridinylthioacetic acid (CAS number 10351-19-6 253.8 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 12, 177 mg, 0.5 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound dark solid bis-sodium salt (218.6 mg, 62%). LCMS (ESI+): R_{f}=3.36, peak failed to ionize; HRMS (ESI−): calculated for C₃₂H₁₉N₂O₈S₃ m/z [M−H]⁻: 655.0309, observed 655.0322; ¹H NMR 300 MHz DMSO δ: 8.15-8.21 (m, 2H), 7.98-8.05 (m, 1H), 7.68-7.73 (m, 1H), 7.46-7.53 (m, 1H), 7.12-7.19 (m, 2H), 4.16-4.26 (m, 2H).

### Example 291—Synthesis of 5-({2-[hydroxy({[2-(2-methoxyethoxy)ethyl]amino})methyl]-1,3-dioxo-2,3-dihydro-1H-inden-5-yl}oxy)-N-[2-(2-methoxyethoxy)ethyl]-1,3-dioxo-2,3-dihydro-1H-indene-2-carboxamide

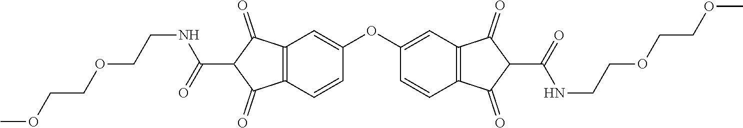

A solution of 5-(2,3-dihydro-1,3-dioxo-1H-inden-6-yloxy)-2,3-dihydro-1,3-dioxo-1H-indene (Example 7, 131 mg, 0.426 mmol) in DMF was cooled to −40° C. in a dry ice MeCN bath and stirred for 5 min. To this was added dropwise, triethylamine (CAS number 121-44-8, 150 ul, 1.07 mmol) and the resulting mixture was stirred for 10 min. To this mixture was then added 1-(2-isocyanatoethoxy)-2-methoxyethane (CAS 90426-82-7, 153 μl, 1.07 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (125.8 mg, 49%). LCMS (ESI+): R_{f}=5.25, M+H=597.75; HRMS (ESI−): calculated for C₃₀H₃₁N₂O₁₁ m/z [M−H]⁻: 595.1933, observed 595.1918; ¹H NMR 300 MHz DMSO δ: 7.57-7.62 (m, 1H), 7.23-7.29 (m, 1H), 7.14-7.18 (m, 1H), 3.38-3.61 8H), 3.21-3.25 (m, 3H).

### Example 292—Synthesis of N-cyclobutyl-5-{[2-(cyclobutylcarbamoyl)-1,3-dioxo-2,3-dihydro-1H-inden-5-yl]sulfonyl}-1,3-dioxo-2,3-dihydro-1H-indene-2-carboxamide

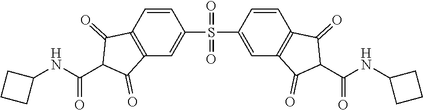 [SMILES: O=C(NC1CCC1)C1C(=O)c2ccc(S(=O)(=O)c3ccc4c(c3)C(=O)C(C(=O)NC3CCC3)C4=O)cc2C1=O]

A solution of 5,5′-sulfonylbis(1H-indene-1,3(2H)-dione) (Example 12, 134.5 mg, 0.38 mmol) in DMF was cooled to −40° C. in a dry ice MeCN bath and stirred for 5 min. To this was added dropwise, triethylamine (CAS number 121-44-8, 150 ul, 1.07 mmol) and the resulting mixture was stirred for 10 min. To this mixture was then added cyclobutyl isocyanate (CAS 5811-25-6, 100 μl, 1.15 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (201.8 mg, 90%). LCMS (ESI+): R_{f}=6.22, M+H=549.62; HRMS (ESI−): calculated for C₂₈H₂₃N₂O₈S m/z [M−H]⁻: 547.1181, observed 547.1183; ¹H NMR 300 MHz DMSO δ: 8.57-8.63 (m, 1H), 8.00-8.06 (m, 1H), 761-7.64 (m, 1H), 7.44-7.49 (m, 1H), 4.26-4.41 (m, 1H), 2.13-2.24 (m, 2H), 1.75-1.89 (m, 2H), 1.54-1.68 (m, 2H).

### Example 293—Synthesis of 5-(2-{1,3-dioxo-2-[2-(2-oxopyrrolidin-1-yl)acetyl]-2,3-dihydro-1H-inden-5-yl}ethynyl)-2-[2-(2-oxopyrrolidin-1-yl)acetyl]-2,3-dihydro-1H-indene-1,3-dione

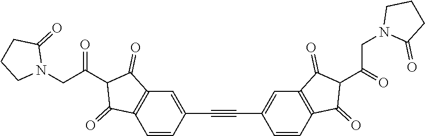 [SMILES: O=C(CN1CCCC1=O)C1C(=O)c2ccc(C#Cc3ccc4c(c3)C(=O)C(C(=O)CN3CCCC3=O)C4=O)cc2C1=O]

To a stirred solution of 2-(2-oxopyrrolidin-1-yl)acetic acid (CAS Regno: 53934-76-2, 680 mg, 4.7 mmol) and DMAP (580 mg, 4.7 mmol) in dry DMF (15 ml) was added EDC·HCl (920 mg, 4.8 mmol). The mixture was stirred at rt for 5 min and 5-[2-(1,3-dioxo-2,3-dihydro-1H-inden-5-yl)ethynyl]-2,3-dihydro-1H-indene-1,3-dione (Example 196, 500 mg, 1.6 mmol) was added. The mixture was stirred at rt overnight and 5% aq HCl (50 ml) was added. After stirring at rt for 15 min, the mixture was vacuum filtered and the dark brown solid collected was dried under high vacuum (970 mg). This solid was stirred with CH₂Cl₂ (10 ml) and 5% aq HCl (10 ml) for 1 hr and filtered. The CH₂Cl₂ layer of the filtrate was separated, dried over Na₂SO₄ and concentrated to leave a solid (70 mg). Prep HPLC gave the title compound (40 mg, 4%) as a yellow solid. ¹H NMR (300 MHz, DMSO-d6) δ: 7.68-7.76 (m, 2H), 7.57 (d, J=0.88 Hz, 2H), 7.48 (d, J=7.47 Hz, 2H), 4.40 (s, 4H), 3.27-3.38 (m, 4H), 2.22 (s, 4H), 1.84-1.99 (m, 4H). LC-MS (ESI+) t_{R} 3.76 min, m/z 565. HRMS (ESI−): calculated for C₃₂H₂₄N₂O₈ m/z [M−H]⁻: 563.1460, observed 563.1459.

### Example 294—Synthesis of N-cyclopentyl-5-{[2-(cyclopentylcarbamoyl)-1,3-dioxo-2,3-dihydro-1H-inden-5-yl]sulfonyl}-1,3-dioxo-2,3-dihydro-1H-indene-2-carboxamide

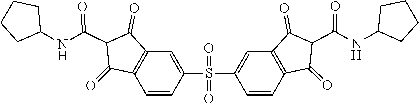 [SMILES: O=C(NC1CCCC1)C1C(=O)c2ccc(S(=O)(=O)c3ccc4c(c3)C(=O)C(C(=O)NC3CCCC3)C4=O)cc2C1=O]

A solution of 5,5′-sulfonylbis(1H-indene-1,3(2H)-dione) (Example 12, 133 mg, 0.375 mmol) in DMF was cooled to −40° C. in a dry ice MeCN bath and stirred for 5 min. To this was added dropwise, triethylamine (CAS number 121-44-8, 150 μl, 1.07 mmol) and the resulting mixture was stirred for 10 min. To this mixture was then added cyclopentyl isocyanate (CAS 4747-71-1, 110 μl, 0.90 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 minutes, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (199.4 mg, 86%). LCMS (ESI+): R_{f}=6.63, M+H=577.63; ¹H NMR 300 MHz DMSO δ: 8.43-8.49 (m, 1H), 8.01-8.07 (m, 1H), 7.59-7.64 (m, 1H), 7.44-7.50 (m, 1H), 4.07-4.19 (m, 1H), 1.76-1.90 (m, 2H), 1.46-1.70 (m, 4H), 1.27-1.42 (m, 2H).

### Example 295—Synthesis of N-[2-(2-methoxyethoxy)ethyl]-5-[(2-{[2-(2-methoxyethoxy) ethyl]carbamoyl}-1,3-dioxo-2,3-dihydro-1H-inden-5-yl)sulfonyl]-1,3-dioxo-2,3-dihydro-1H-indene-2-carboxamide

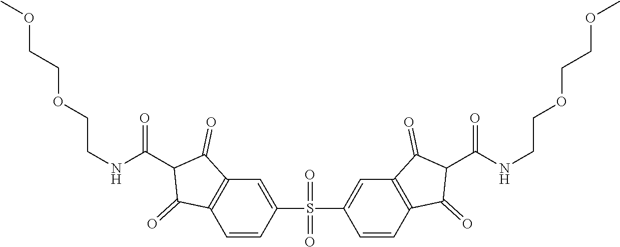

A solution of 5,5′-sulfonylbis(1H-indene-1,3(2H)-dione) (Example 11, 133 mg, 0.375 mmol) in DMF was cooled to −40° C. in a dry ice MeCN bath and stirred for 5 min. To this was added dropwise, triethylamine (CAS number 121-44-8, 150 μl, 1.07 mmol) and the resulting mixture was stirred for 10 min. To this mixture was then added 1-(2-Isocyanatoethoxy)-2-methoxyethane (CAS 90426-82-7, 153 μl, 1.07 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (196.9 mg, 76%). LCMS (ESI+): R_{f}=4.88, M+H=645.74; HRMS (ESI−): calculated for C₃₀H₃₁N₂O₁₂S m/z [M−H]⁻: 643.1603, observed 643.1616; H NMR 300 MHz DMSO δ: 8.48-8.55 (m, 11H), 8.03-8.08 (m, 1H), 7.64-7.67 (m, 1H), 7.47-7.67 (m, 1H), 3.48-3.55 (m, 2H), 3.44 (dd, J=3.51, 5.27 Hz, 4H), 3.39 (br. S., 1H), 3.33-3.39 (m, 2H), 3.21-3.26 (m, 3H).

### Example 296—Synthesis of 2-cyclobutanecarbonyl-5-[(2-cyclobutanecarbonyl-1,3-dioxo-2,3-dihydro-1H-inden-5-yl)sulfonyl]-2,3-dihydro-1H-indene-1,3-dione

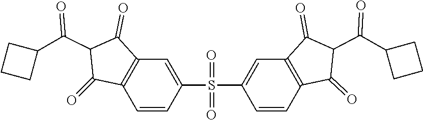 [SMILES: O=C1c2ccc(S(=O)(=O)c3ccc4c(c3)C(=O)C(C(=O)C3CCC3)C4=O)cc2C(=O)C1C(=O)C1CCC1]

To a solution of dimethylaminopyridine (DMAP, 183 mg, 1.50 mmol), cyclobutanecarboxylic acid (CAS number 3721-95-7, 150.2 mg, 1.50 mmol) in anhydrous DMF (10 ml) was added EDCI·HCl (286.5 mg, 1.50 mmol) and the contents were stirred for 10 min. 5,5′-sulfonylbis(1H-indene-1,3(2H)-dione) (Example 11, 177 mg, 0.5 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (201.4 mg, 77%). LCMS (ESI+): R_{f}=7.10, M+H=519.58; HRMS (ESI−): calculated for C₂₈H₂₁O₈S m/z [M−H]⁻: 517.0963, observed 517.0945; ¹H NMR 300 MHz DMSO δ: 8.29-8.34 (m, 1H), 8.09 (d, J=1.17 Hz, 1H), 7.79-7.84 (m, 1H), 4.17-4.32 (m, 1H), 2.05-2.29 (m, 5H), 1.91-2.03 (m, 1H), 1.73-1.86 (m, 1H).

### Example 297—Synthesis of 2-cyclobutanecarbonyl-5-[(2-cyclobutanecarbonyl-1,3-dioxo-2,3-dihydro-1H-inden-5-yl)oxy]-2,3-dihydro-1H-indene-1,3-dione

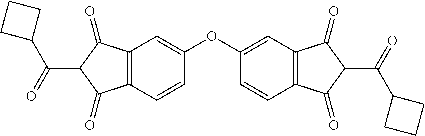 [SMILES: O=C1c2ccc(Oc3ccc4c(c3)C(=O)C(C(=O)C3CCC3)C4=O)cc2C(=O)C1C(=O)C1CCC1]

To a solution of dimethylaminopyridine (DMAP, 183 mg, 1.50 mmol), cyclobutanecarboxylic acid (CAS number 3721-95-7, 150.2 mg, 1.50 mmol) in anhydrous DMF (10 ml) was added EDCI·HCl (286.5 mg, 1.50 mmol) and the contents were stirred for min. 5-(2,3-dihydro-1,3-dioxo-1H-inden-6-yloxy)-2,3-dihydro-1,3-dioxo-1H-indene (Example 7, 159.1 mg, 0.520 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (143.0 mg, 54%). LCMS (ESI+): R_{f}=7.57, M+H=471.65; HRMS (ESI−): calculated for C₂₈H₂₁O₇ m/z [M−H]⁻: 469.1293, observed 469.1283; ¹H NMR 300 MHz DMSO δ: 7.34-7.38 (m, 1H), 7.08 (dd, J=2.34, 7.62 Hz, 1H), 6.86-6.88 (m, 1H), 4.11-4.24 (m, 1H), 2.03-2.16 (m, 2H), 1.91-2.03 (m, 3H), 1.80-1.91 (m, 1H), 1.64-1.74 (m, 1H).

### Example 298—Synthesis of N-[2-(5-{[2-(2-acetamidoacetyl)-1,3-dioxo-2,3-dihydro-1H-inden-5-yl]oxy}-1,3-dioxo-2,3-dihydro-1H-inden-2-yl)-2-oxoethyl]acetamide

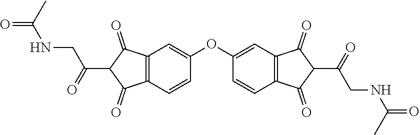 [SMILES: CC(=O)NCC(=O)C1C(=O)c2ccc(Oc3ccc4c(c3)C(=O)C(C(=O)CNC(C)=O)C4=O)cc2C1=O]

To a solution of dimethylaminopyridine (DMAP, 183 mg, 1.50 mmol), N-acetyl-Glycine (CAS number 543-24-8, 176.5 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 157.1 mg, 0.513 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (145.2 mg, 56%). LCMS (ESI+): R_{f}=3.92, M+H=505.61; HRMS (ESI−): calculated for C₂₆H₁₉N₂O₉ m/z [M−H]⁻: 503.1096, observed 503.1087; ¹H NMR 300 MHz DMSO δ: 7.86-7.93 (bs, 1H), 7.57-7.63 (m, 1H), 7.25-7.31 (m, 1H), 7.09-7.12 (m, 1H), 4.31-4.40 (m, 3H), 1.83-1.90 (m, 3H).

### Example 299—Synthesis of 2-cyclobutanecarbonyl-5-(2-cyclobutanecarbonyl-1,3-dioxo-2,3-dihydro-1H-indene-5-carbonyl)-2,3-dihydro-1H-indene-1,3-dione

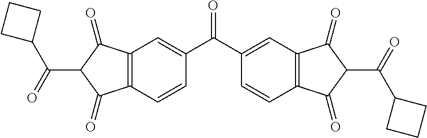 [SMILES: O=C(c1ccc2c(c1)C(=O)C(C(=O)C1CCC1)C2=O)c1ccc2c(c1)C(=O)C(C(=O)C1CCC1)C2=O]

To a solution of dimethylaminopyridine (DMAP, 183 mg, 1.50 mmol), cyclobutanecarboxylic acid (CAS number 3721-95-7, 150.2 mg, 1.50 mmol) in anhydrous DMF (10 ml) was added EDCI·HCl (286.5 mg, 1.50 mmol) and the contents were stirred for 10 min. 5,5′-carbonylbis(1H-indene-1,3(2H)-dione (Example 4, 159 mg, 0.5 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1 N NaOH and 10 ml of methylene chloride. This biphasic suspension was shaken vigorously and filtered to provide the title compound as the dark solid bis-sodium salt (226.0 mg, 86%). LCMS (ESI+): R_{f}=7.24, M+H=483.67; HRMS (ESI−): calculated for C₂₉H₂₁O₇ m/z [M−H]⁻: Exact Mass: 481.1293, observed 481.1274; ¹H NMR 300 MHz DMSO δ: 7.81-7.84 (m, 1H), 7.47-7.52 (m, 1H), 4.14-4.24 (m, 2H), 1.93-2-05 (m, 3H), 1.82-1.92 (m, 1H), 1.62-1.75 (m, 1H).

### Example 300—Synthesis of N-cyclopropyl-5-{2-[2-(cyclopropylcarbamoyl)-1,3-dioxo-2,3-dihydro-1H-inden-5-yl]ethynyl}-1,3-dioxo-2,3-dihydro-1H-indene-2-carboxamide

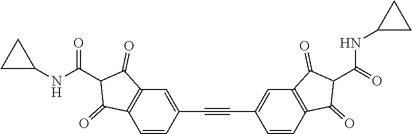 [SMILES: O=C(NC1CC1)C1C(=O)c2ccc(C#Cc3ccc4c(c3)C(=O)C(C(=O)NC3CC3)C4=O)cc2C1=O]

A stirred mixture of 5-[2-(1,3-dioxo-2,3-dihydro-1H-inden-5-yl)ethynyl]-2,3-dihydro-1H-indene-1,3-dione (Example 196, 120 mg, 0.38 mmol) and dry DMF (5 ml) was cooled in a dry ice/acetone bath and cyclopropyl isocyanate (CAS Regno: 4747-72-2, 0.12 ml, 1.5 mmol) was added dropwise over 2 min, followed by Et₃N (0.27 ml, 1.9 mmol) also dropwise over 2 min. The dry ice/acetone bath was allowed to expire and the mixture was stirred overnight at rt. The mixture was diluted with 5% aq HCl (25 ml), stirred at rt for 30 min and vacuum filtered. The dark solid collected was stirred with CH₂Cl₂ (10 ml) and 5% aq HCl (10 ml) at rt for 30 min and filtered. The CH₂Cl₂ layer of the filtrate was concentrated to leave a brown residue which was stirred with 1 M aq NaOH (10 ml) and CH₂Cl₂ (10 ml). The mixture was filtered and a black solid was collected. This solid was successively triturated with CH₂Cl₂ (10 ml), 1:1 CH₂Cl₂/MeOH (10 ml) and MeOH (10 ml). The supernatant from the CH₂Cl₂/MeOH trituration was concentrated to leave the disodium salt of the title compound (13 mg, 6%). ¹H NMR (300 MHz, DMSO-d6) δ: 8.49 (d, J=3.52 Hz, 2H), 7.59 (dd, J=7.47, 1.32 Hz, 2H), 7.31 (d, J=7.47 Hz, 2H), 2.67 (br dd, J=7.25, 3.30 Hz, 2H), 0.53-0.74 (m, 4H), 0.28-0.50 (m, 4H)., LC-MS (ESI+) t_{R} 5.98 min, m/z 481. HRMS (ESI−): calculated for C₂₈H₂₀N₂O₆ m/z [M−H]⁻: 479.1249, observed 479.1236.

### Example 301—Synthesis of 6-acetyl-3-({6-acetyl-5,7-dioxo-5H,6H,7H-cyclopenta[b]pyridin-3-yl}sulfonyl)-5H,6H,7H-cyclopenta[b]pyridine-5,7-dione

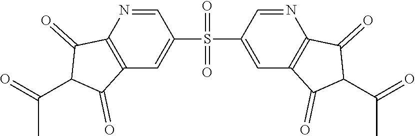 [SMILES: CC(=O)C1C(=O)c2cc(S(=O)(=O)c3cnc4c(c3)C(=O)C(C(C)=O)C4=O)cnc2C1=O]

Step 1; A mixture of 2,3-dimethyl 5-bromopyridine-2,3-dicarboxylate (CAS Regno: 521980-82-5, 464 mg, 1.7 mmol), Na₂S·xH₂O (CAS Regno: 27610-45-3, 205 mg, 0.85 mmol assuming x=9) and DMF (10 ml) was stirred at 50° C. for 2 hr and at 75° C. for 16 hr. The mixture was diluted with EtOAc (90 ml), washed with water (10 ml) and brine (10 ml), and dried over Na₂SO₄. Removal of the solvent left an oil (270 mg) which was chromatographed on a 40 g silica cartridge, eluted with a 0-70% EtOAc in hexanes gradient, to give 2,3-dimethyl 5-{[5,6-bis(methoxycarbonyl)pyridin-3-yl]sulfanyl}pyridine-2,3-dicarboxylate (100 mg, 28%) as a white solid. ¹H NMR (300 MHz, CHLOROFORM-d) 5: 8.63-8.73 (m, 2H), 8.02 (d, J=2.20 Hz, 2H), 3.96 (s, 6H), 3.89 (s, 6H). LC-MS (ESI+) t_{R} 4.13 min, m/z 421.
Step 2; To a stirred solution of 2,3-dimethyl 5-{[5,6-bis(methoxycarbonyl)pyridin-3-yl]sulfanyl}pyridine-2,3-dicarboxylate (100 mg, 0.24 mmol) in CH₂Cl₂ (5 ml) was added solid≤77% m-CPBA (937-14-4, 120 mg, ≤0.53 mmol). The mixture was stirred at rt for 18 hr, diluted with CH₂Cl₂ (40 ml), washed with saturated aqueous NaHCO₃ (5 ml) and brine (5 ml), and dried over Na₂SO₄. The solvent was removed and the white solid residue was chromatographed on a 24 g silica cartridge, eluted with a 0-100% EtOAc in hexanes gradient, to give 2,3-dimethyl 5-{[5,6-bis(methoxycarbonyl)pyridin-3-yl]sulfonyl}pyridine-2,3-dicarboxylate (70 mg, 65%) as a white solid. ¹H NMR (300 MHz, CHLOROFORM-d) 9.21-9.35 (m, 2H), 8.72 (d, J=2.20 Hz, 2H), 4.00 (s, 6H), 3.97 (s, 6H). LC-MS (ESI+) t_{R} 4.21 min, m/z 453.

Step 3: To a stirred solution of 2,3-dimethyl 5-{[5,6-bis(methoxycarbonyl)pyridin-3-yl]sulfonyl}pyridine-2,3-dicarboxylate (70 mg, 0.16 mmol) in MeOH (3 ml) was added 1 N aq NaOH (2 ml, 2.0 mmol). The mixture was stirred at rt overnight and concentrated. The residue was taken up in 1:1 5% aq HCl/MeCN and concentrated under reduced pressure. The residue was taken up 1:3 water/MeCN and evaporated to dryness under reduced pressure to provide a white solid. Toluene (2 ml) and SOCl₂ (0.25 ml, mmol). The mixture was heated at reflux overnight during which time solvent was lost. The residue was assumed to be 3-({5,7-dioxo-5H,7H-furo[3,4-b]pyridin-3-yl}sulfonyl)-5H,7H-furo[3,4-b]pyridine-5,7-dione and was used directly.

Step 4: Crude 3-({5,7-dioxo-5H,7H-furo[3,4-b]pyridin-3-yl}sulfonyl)-5H,7H-furo[3,4-b]pyridine-5,7-dione (50.16 mmol) from Step 3 was stirred with acetic anhydride (1 ml) and 2,4-pentanedione (40 mg, 0.40 mmol) and Et₃N (0.2 ml, 1.45 mmol) was added. The mixture was stirred at rt for 1 h and 3% aq HCl (10 ml) was added. Stirring was continued for 2 h and the mixture was filtered. After drying under vacuum, the brown solid collected (40 mg) was sonicated with EtOAc (5 ml) and centrifuged. The supernatant was removed and the residue was dried under vacuum to give 80% pure title compound (31 mg, 43% over 2 steps). ¹H NMR (300 MHz, DMSO-d6) δ: 9.36-9.51 (m, 2H), 8.54 (d, J=1.76 Hz, 2H), 2.41 (s, 6H). LC-MS (ESI+) t_{R} 2.58 min, m/z 441. HRMS (ESI−): calculated for C₂₀H₁₂N₂O₈S m/z [M−H]⁻: 439.0242, observed 439.0243.

### Example 302—Synthesis of ethyl 3-{[5-({2-[(3-ethoxy-3-oxopropyl)carbamoyl]-1,3-dioxo-2,3-dihydro-1H-inden-5-yl}sulfonyl)-1,3-dioxo-2,3-dihydro-1H-inden-2-yl]formamido}propanoate

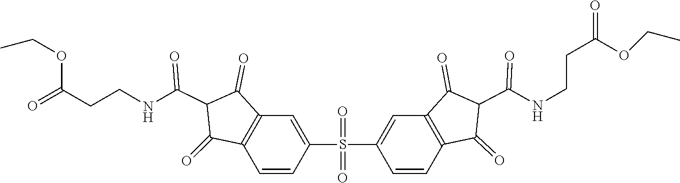 [SMILES: CCOC(=O)CCNC(=O)C1C(=O)c2ccc(S(=O)(=O)c3ccc4c(c3)C(=O)C(C(=O)NCCC(=O)OCC)C4=O)cc2C1=O]

A solution of 5,5′-sulfonylbis(1H-indene-1,3(2H)-dione) (Example 11, 179 mg, 0.497 mmol) in DMF was cooled to −40° C. in a dry ice MeCN bath and stirred for 5 minutes. To this was added dropwise, triethylamine (180 μl, 1.28 mmol) and the resulting mixture was stirred for 10 min. To this was then added Ethyl 3-isocyanatopropanoate (CAS 5100-34-5, 165 μl, 1.25 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (230.6 mg, 67%). HRMS (ESI−): calculated for C₃₀H₂₇N₂O₁₂S m/z [M−H]⁻: 639.1290, observed 639.1305; ¹H NMR 300 MHz DMSO δ: 8.47-8.54 (m, 1H), 8.00-8.05 (m, 1H), 7.60-7.64 (m, 1H), 7.43-7.49 (m, 1H), 4.00-4.10 (m, 2H), 3.25-3.35 (m, 2H), 2.15-2.50 (m, 2H), 1.10-1.20 (m, 3H).

### Example 303—Synthesis of 3-{[5-({2-[(2-carboxyethyl)carbamoyl]-1,3-dioxo-2,3-dihydro-1H-inden-5-yl}sulfonyl)-1,3-dioxo-2,3-dihydro-1H-inden-2-yl]formamido}propanoic acid

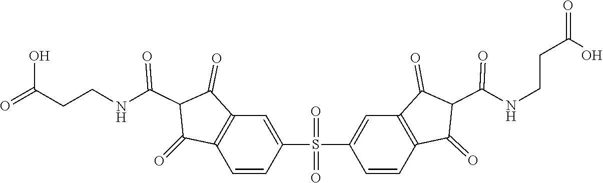 [SMILES: O=C(O)CCNC(=O)C1C(=O)c2ccc(S(=O)(=O)c3ccc4c(c3)C(=O)C(C(=O)NCCC(=O)O)C4=O)cc2C1=O]

To a 40 ml vial equipped with a stirbar was charged ethyl 3-{[5-({2-[(3-ethoxy-3-oxopropyl)carbamoyl]-1,3-dioxo-2,3-dihydro-1H-inden-5-yl}sulfonyl)-1,3-dioxo-2,3-dihydro-1H-inden-2-yl]formamido}propanoate (Example 302, 221 mg, 0.323 mmol) and 5 ml of MeOH to produce a dark slurry. To this was added 5 ml of 1 N NaOH and the resulting mixture was allowed to stir overnight at ambient temperature. Once the reaction was deemed complete by LCMS, the reaction was transferred to a water bath and 5 ml of 2N HCl was added dropwise, causing a precipitate to form. The slurry was stirred for 5 min, then the solid was isolated by filtration. The solid was washed with 10 ml of H₂O, then dried in the vacuum oven at 45° C. and full house vacuum to provide the title compound as the dark brown free acid (163.5 mg, 86%). LCMS (ESI+): R_{f}=3.95, M+H=585.37; HRMS (ESI−): calculated for C₂₆H₁₉N₂O₁₂S m/z [M−H]⁻: 583.0664, observed 583.0678; ¹H NMR 300 MHz DMSO δ: 9.10-9.05 (m, 1H), 8.33-8.41 (m, 1H), 7.78-7.86 (m, 1H), 3.58-3.70 (m, 2H), 2.64-2.72 (m, 2H).

### Example 304—Synthesis of ethyl 3-{[5-({2-[(3-ethoxy-3-oxopropyl)carbamoyl]-1,3-dioxo-2,3-dihydro-1H-inden-5-yl}oxy)-1,3-dioxo-2,3-dihydro-1H-inden-2-yl]formamido}propanoate

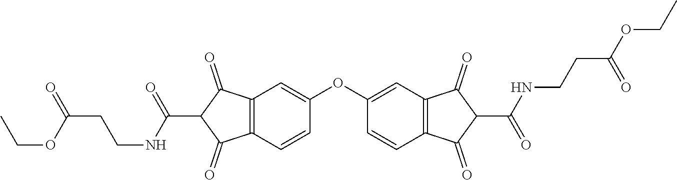 [SMILES: CCOC(=O)CCNC(=O)C1C(=O)c2ccc(Oc3ccc4c(c3)C(=O)C(C(=O)NCCC(=O)OCC)C4=O)cc2C1=O]

A solution of 5-(2,3-dihydro-1,3-dioxo-1H-inden-6-yloxy)-2,3-dihydro-1,3-dioxo-1H-indene (Example 7, 156 mg, 0.510 mmol) in DMF was cooled to −40° C. in a dry ice MeCN bath and stirred for 5 min. To this was added dropwise, triethylamine (165 μl, 1.28 mmol) and the resulting mixture was stirred for 10 min. To this mixture was then added 3-isocyanatopropanoic acid ethyl ester (CAS 5100-34-5, 165 μl, 1.25 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (261.6 mg, 80%). LCMS (ESI+): R_{f}=5.98, M+H=593.12; HRMS (ESI−): calculated for C₃₀H₂₇N₂O₁₁ m/z [M−H]⁻: 591.1620, observed 591.1623; ¹H NMR 300 MHz DMSO δ: 8.60-8.69 (m, 1H), 7.34-7.42 (m, 1H), 7.07-7.13 (m, 1H), 6.89-6.94 (m, 1H), 4.08-4.21 (m, 2H), 3.45-3.56 (m, 2H), 1.21-1.32 (m, 3H).

### Example 305—Synthesis of 3-{[5-({2-[(2-carboxyethyl)carbamoyl]-1,3-dioxo-2,3-dihydro-1H-inden-5-yl}oxy)-1,3-dioxo-2,3-dihydro-1H-inden-2-yl]formamido}propanoic acid

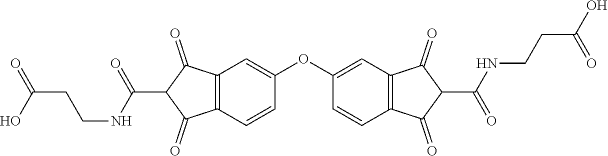 [SMILES: O=C(O)CCNC(=O)C1C(=O)c2ccc(Oc3ccc4c(c3)C(=O)C(C(=O)NCCC(=O)O)C4=O)cc2C1=O]

To a 40 ml vial equipped with a stirbar was charged ethyl 3-{[5-({2-[(3-ethoxy-3-oxopropyl)carbamoyl]-1,3-dioxo-2,3-dihydro-1H-inden-5-yl}oxy)-1,3-dioxo-2,3-dihydro-1H-inden-2-yl]formamido}propanoate (Example 304, 144 mg, 0.226 mmol) and 5 ml of MeOH to produce a dark slurry. To this was added 5 ml of 1N NaOH and the resulting mixture was allowed to stir overnight at ambient temperature. Once the reaction was deemed complete by LCMS, the reaction was transferred to a water bath and 5 ml of 2N HCl was added dropwise, causing a precipitate to form. The slurry was stirred for 5 min, then the solid was isolated by filtration. The solid was washed with 10 ml of H₂O, then dried in the vacuum oven at 45° C. and full house vacuum to provide the title compound as the dark brown free acid (53.9 mg, 44%). LCMS (ESI+): R_{f}=4.47, M+H=537.34; HRMS (ESI−): calculated for C₂₆H₁₉NO₁₁ m/z [M−H]⁻: 535.0994, observed 535.1008; ¹H NMR 300 MHz DMSO δ: 8.78-8.95 (m, 1H), 7.68-7.81 (m, 1H), 7.34-7.45 (m, 1H), 7.26-7.33 (m, 1H), 3.58-3.75 (m, 2H), 2.64-2.78 (m, 2H).

### Example 306—Synthesis of ethyl 2-{[5-({2-[(2-ethoxy-2-oxoethyl)carbamoyl]-1,3-dioxo-2,3-dihydro-1H-inden-5-yl}oxy)-1,3-dioxo-2,3-dihydro-1H-inden-2-yl]formamido}acetate

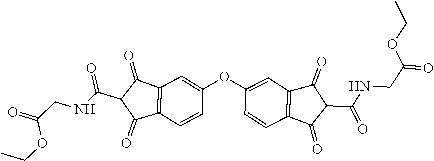 [SMILES: CCOC(=O)CNC(=O)C1C(=O)c2ccc(Oc3ccc4c(c3)C(=O)C(C(=O)NCC(=O)OCC)C4=O)cc2C1=O]

A solution of 5-(2,3-dihydro-1,3-dioxo-1H-inden-6-yloxy)-2,3-dihydro-1,3-dioxo-1H-indene (Example 7, 158 mg, 0.515 mmol) in DMF was cooled to −40° C. in a dry ice MeCN bath and stirred for 5 min. To this was added dropwise, triethylamine (180 μl, 1.28 mmol) and the resulting mixture was stirred for 10 min. To this mixture was then added ethyl isocyanatoacetate (CAS 2949-22-6, 150 μl, 1.34 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (269.2 mg, 86%). LCMS (ESI+): R_{f}=5.86, M+H=565.16; HRMS (ESI−): calculated for C₂₈H₂₃N₂O₁₁ m/z [M−H]⁻: 563.1307, observed 563.1301; ¹H NMR 300 MHz DMSO δ: 8.76-8.85 (m, 1H), 7.08-7.15 (m, 1H), 6.93-6.99 (m, 1H), 4.11-4.23 (m, 2H), 4.02-4.09 (m, 2H), 1.22-1.31 (m, 3H).

### Example 307—Synthesis of 2-{[5-({2-[(carboxymethyl)carbamoyl]-1,3-dioxo-2,3-dihydro-1H-inden-5-yl}oxy)-1,3-dioxo-2,3-dihydro-1H-inden-2-yl]formamido}acetic acid

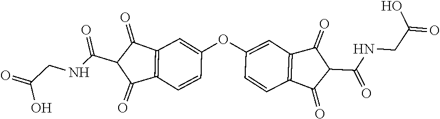 [SMILES: O=C(O)CNC(=O)C1C(=O)c2ccc(Oc3ccc4c(c3)C(=O)C(C(=O)NCC(=O)O)C4=O)cc2C1=O]

To a 40 ml vial equipped with a stirbar was charged ethyl 2-{[5-({2-[(2-ethoxy-2-oxoethyl)carbamoyl]-1,3-dioxo-2,3-dihydro-1H-inden-5-yl}oxy)-1,3-dioxo-2,3-dihydro-1H-inden-2-yl]formamido}acetate (Example 306, 159.5 mg, 0.262 mmol) and 5 ml of MeOH to produce a dark slurry. To this was added 5 ml of 1N NaOH and the resulting mixture was allowed to stir overnight at ambient temperature. Once the reaction was deemed complete by LCMS, the reaction was transferred to a water bath and 5 ml of 2N HCl was added dropwise, causing a precipitate to form. The slurry was stirred for 5 min, then the solid was isolated by filtration. The solid was washed with 10 ml of H₂O, then dried in the vacuum oven at 45° C. and full house vacuum to provide the title compound as the dark brown free acid (118.5 mg, 89%). LCMS (ESI+): R_{f}=4.17, M+H=509.24; HRMS (ESI−): calculated for C₂₄H₁₅N₂O₁₁ m/z [M−H]⁻: 507.0681, observed 507.0683; ¹H NMR 300 MHz DMSO δ: 8.78-9.26 (bs, 1H), 7.68-7.79 (m, 1H), 7.24-7.44 (m, 2H), 4.09-4.23 (s, 2H).

### Example 308—Synthesis of ethyl 2-[(5-{2-[(2-ethoxy-2-oxoethyl)carbamoyl]-1,3-dioxo-2,3-dihydro-1H-indene-5-carbonyl}-1,3-dioxo-2,3-dihydro-1H-inden-2-yl)formamido]acetate

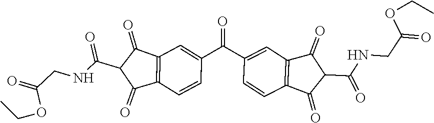 [SMILES: CCOC(=O)CNC(=O)C1C(=O)c2ccc(C(=O)c3ccc4c(c3)C(=O)C(C(=O)NCC(=O)OCC)C4=O)cc2C1=O]

A solution of 5,5′-carbonylbis(1H-indene-1,3(2H)-dione (Example 4, 161.4 mg, 0.507 mmol) in DMF was cooled to −40° C. in a dry ice MeCN bath and stirred for 5 min. To this was added dropwise, triethylamine (180 μl, 1.28 mmol) and the resulting mixture was stirred for 10 min. To this mixture was then added ethyl isocyanatoacetate (CAS 2949-22-6, 150 μl, 1.34 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (248.4 mg, 79%). LCMS (ESI+): R_{f}=5.61, M+H=577.20; HRMS (ESI−): calculated for C₂₉H₂₃N₂O₁₁ m/z [M−H]⁻: 575.1307, observed 575.1321; ¹H NMR 300 MHz DMSO δ: 8.80-8.87 (m, 1H), 7.86-7.92 (m, 1H), 7.63-7.66 (m, 1H), 7.52-7.57 (m, 1H), 4.10-4.21 (m, 2H), 4.02-4.09 (m, 2H), 1.21-1.32 (m, 4H).

### Example 309—Synthesis of 2-[(5-{2-[(carboxymethyl)carbamoyl]-1,3-dioxo-2,3-dihydro-1H-indene-5-carbonyl}-1,3-dioxo-2,3-dihydro-1H-inden-2-yl)formamido]acetic acid

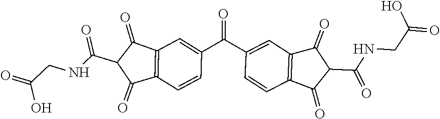 [SMILES: O=C(O)CNC(=O)C1C(=O)c2ccc(C(=O)c3ccc4c(c3)C(=O)C(C(=O)NCC(=O)O)C4=O)cc2C1=O]

To a 40 ml vial equipped with a stirbar was charged ethyl 2-[(5-{2-[(2-ethoxy-2-oxoethyl)carbamoyl]-1,3-dioxo-2,3-dihydro-1H-indene-5-carbonyl}-1,3-dioxo-2,3-dihydro-1H-inden-2-yl)formamido]acetate (Example 308, 154.8 mg, 0.254 mmol) and 5 ml of MeOH to produce a dark slurry. To this was added 5 ml of 1N NaOH and the resulting mixture was allowed to stir overnight at ambient temperature. Once the reaction was deemed complete by LCMS, the reaction was transferred to a water bath and 5 ml of 2N HCl was added dropwise, causing a precipitate to form. The slurry was stirred for 5 min, then the solid was isolated by filtration. The solid was washed with 10 ml of H₂O, then dried in the vacuum oven at 45° C. and full house vacuum to provide the title compound as the dark brown free acid (113.9 mg, 86%). LCMS (ESI+): R_{f}=3.76, M+H=521.08; HRMS (ESI−): calculated for C₂₅H₁₅N₂O₁₁ m/z [M−H]⁻: 519.0681, observed 519.0694; ¹H NMR 300 MHz DMSO δ: 8.03-8.10 (m, 1H), 7.86-7.91 (m, 1H), 7.78-7.84 (m, 1H), 4.13-4.22 (s, 2H).

### Example 310—Synthesis of ethyl 2-{[5-({2-[(2-ethoxy-2-oxoethyl)carbamoyl]-1,3-dioxo-2,3-dihydro-1H-inden-5-yl}sulfonyl)-1,3-dioxo-2,3-dihydro-1H-inden-2-yl]formamido}acetate

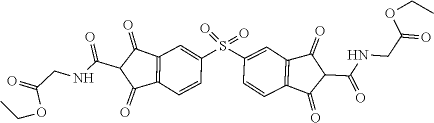 [SMILES: CCOC(=O)CNC(=O)C1C(=O)c2ccc(S(=O)(=O)c3ccc4c(c3)C(=O)C(C(=O)NCC(=O)OCC)C4=O)cc2C1=O]

A solution of 5,5′-sulfonylbis(1H-indene-1,3(2H)-dione) (Example 11, 177 mg, 0.5 mmol) in DMF was cooled to −4° C. in a dry ice MeCN bath and stirred for 5 min. To this was added dropwise, triethylamine (180 μl, 1.28 mmol) and the resulting mixture was stirred for 10 min. To this mixture was then added ethyl isocyanatoacetate (CAS 2949-22-6, 150 μl, 1.34 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (219.4 mg, 67%). LCMS (ESI+): R_{f}=5.32, M+H=612.79; HRMS (ESI−): calculated for C₂₈H₂₃N₂O₁₂S m/z [M−H]⁻: 611.0977, observed 611.0998; ¹H NMR 300 MHz DMSO δ: 8.69-8.76 (m, 1H), 8.05-8.13 (m, 2H), 7.66-7.73 (m, 1H), 7.50-7.56 (m, 1H), 4.08-4.17 (m, 2H), 3.96-4.05 (m, 2H), 1.18-1.27 (m, 3H).

### Example 311—Synthesis of 2-{[5-({2-[(carboxymethyl)carbamoyl]-1,3-dioxo-2,3-dihydro-1H-inden-5-yl}sulfonyl)-1,3-dioxo-2,3-dihydro-1H-inden-2-yl]formamido}acetic acid

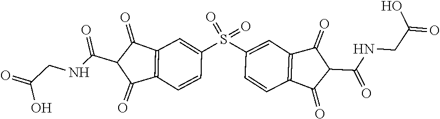 [SMILES: O=C(O)CNC(=O)C1C(=O)c2ccc(S(=O)(=O)c3ccc4c(c3)C(=O)C(C(=O)NCC(=O)O)C4=O)cc2C1=O]

To a 40 ml vial equipped with a stirbar was charged ethyl 2-{[5-({2-[(2-ethoxy-2-oxoethyl)carbamoyl]-1,3-dioxo-2,3-dihydro-1H-inden-5-yl}sulfonyl)-1,3-dioxo-2,3-dihydro-1H-inden-2-yl]formamido}acetate (Example 310, 145.7 mg, 0.235 mmol) and 5 ml of MeOH to produce a dark slurry. To this was added 5 ml of 1N NaOH and the resulting mixture was allowed to stir overnight at ambient temperature. Once the reaction was deemed complete by LCMS, the reaction was transferred to a water bath and 5 ml of 2N HCl was added dropwise, causing a precipitate to form. The slurry was stirred for 5 min, then the solid was isolated by filtration. The solid was washed with 10 ml of H₂O, then dried in the vacuum oven at 45° C. and full house vacuum to provide the title compound as the dark brown free acid (111 mg, 85%). LCMS (ESI+): R_{f}=3.47, M+H=557.27; HRMS (ESI−): calculated for C₂₄H₁₅N₂O₁₂S m/z [M−H]⁻: 555.0351, observed 555.0356; ¹H NMR 300 MHz DMSO δ: 8.30-8.36 (m, 1H), 7.98-8.03 (m, 1H) 7.76-7.82 (m, 1H), 4.10-4.15 (m, 2H).

### Example 312—Synthesis of methyl (2S)-2-({5-[(2-{[(2S)-1-methoxy-1-oxopropan-2-yl]carbamoyl}-1,3-dioxo-2,3-dihydro-1H-inden-5-yl)oxy]-1,3-dioxo-2,3-dihydro-1H-inden-2-yl}formamido)propanoate

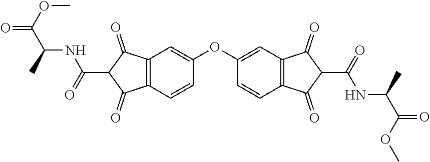 [SMILES: COC(=O)[C@H](C)NC(=O)C1C(=O)c2ccc(Oc3ccc4c(c3)C(=O)C(C(=O)N[C@@H](C)C(=O)OC)C4=O)cc2C1=O]

A solution of 5-(2,3-dihydro-1,3-dioxo-1H-inden-6-yloxy)-2,3-dihydro-1,3-dioxo-1H-indene (Example 7, 158 mg, 0.515 mmol) in DMF was cooled to −40° C. in a dry ice MeCN bath and stirred for 5 min. To this was added dropwise, triethylamine (180 μl, 1.28 mmol) and the resulting mixture was stirred for 10 min. To this mixture was then added (S)-(−)-2-isocyanatopropionic acid methyl ester (CAS 30293-82-4, 150 μl, 1.30 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (241.6 mg, 79%). LCMS (ESI+): R_{f}=5.88, M+H=565.09; HRMS (ESI−): calculated for C₂₈H₂₃N₂O₁₁ m/z [M−H]⁻: 563.1307, observed 563.1321; ¹H NMR 300 MHz DMSO δ: 8.81-8.89 (m, 1H), 7.35-7.42 (m, 1H), 6.90-6.96 (m, 1H), 4.45-4.57 (m, 1H), 3.66-3.73 (m, 3H), 1.34-1.45 (m, 3H).

### Example 313—Synthesis of (2S)-2-({5-[(2-{[(1S)-1-carboxyethyl]carbamoyl}-1,3-dioxo-2,3-dihydro-1H-inden-5-yl)oxy]-1,3-dioxo-2,3-dihydro-1H-inden-2-yl}formamido) propanoic acid

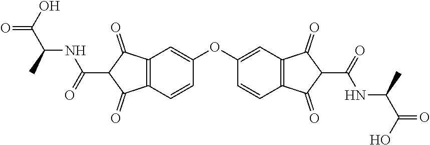 [SMILES: C[C@H](NC(=O)C1C(=O)c2ccc(Oc3ccc4c(c3)C(=O)C(C(=O)N[C@@H](C)C(=O)O)C4=O)cc2C1=O)C(=O)O]

To a 40 ml vial equipped with a stirbar was charged methyl (2S)-2-({5-[(2-{[(2S)-1-methoxy-1-oxopropan-2-yl]carbamoyl}-1,3-dioxo-2,3-dihydro-1H-inden-5-yl)oxy]-1,3-dioxo-2,3-dihydro-1H-inden-2-yl}formamido)propanoate (Example 312, 138.8 mg, 0.211 mmol) and 5 ml of MeOH to produce a dark slurry. To this was added 5 ml of 1N NaOH and the resulting mixture was allowed to stir overnight at ambient temperature. Once the reaction was deemed complete by LCMS, the reaction was transferred to a water bath and 5 ml of 2N HCl was added dropwise, causing a precipitate to form. The slurry was stirred for 5 min, then the solid was isolated by filtration. The solid was washed with 10 ml of H₂O, then dried in the vacuum oven at 45° C. and full house vacuum to provide the title compound as the dark brown free acid (100.8 mg, 89%). LCMS (ESI+): R_{f}=4.69, M+H=537.27; HRMS (ESI−): calculated for C₂₆H₁₉N₂O₁₁ m/z [M−H]⁻: 535.0994, observed 535.0995; ¹H NMR 300 MHz DMSO δ: 7.69-7.76 (m, 1H), 7.35-7.42 (m, 1H), 7.27-7.32 (m, 1H), 4.55-4.67 (m, 1H), 1.48-1.60 (m, 3H).

### Example 314—Synthesis of -acetyl-N-(2-acetyl-1,3-dioxo-2,3-dihydro-1H-inden-5-yl)-N-methyl-1,3-dioxo-2,3-dihydro-1H-indene-5-sulfonamide

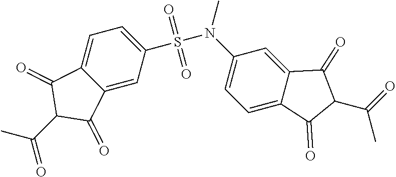 [SMILES: CC(=O)C1C(=O)c2ccc(N(C)S(=O)(=O)c3ccc4c(c3)C(=O)C(C(C)=O)C4=O)cc2C1=O]

Step 1: To a stirred, ice-cold solution mixture of 1,2-dimethyl 4-aminobenzene-1,2-dicarboxylate (CAS Regno: 51832-31-6, 570 mg, 2.7 mmol) EtOAc (2.5 ml) and conc HCl (1.5 ml) was added dropwise over 2 min, a solution of NaNO₂ (197 mg, 2.9 mmol) in water (1.5 ml). The mixture was stirred in the ice bath for 45 min and a slurry of CuCl₂·2H₂O (211 mg, 1.2 mmol), NaHSO₃ (2.84 g, 27.3 mmol), HOAc (3.5 ml) was added. Conc HCl (˜1.5 ml), HOAc (˜3 ml) and water (˜2 ml) were used to help wash the slurry into the reaction mixture. The mixture was stirred at rt for 1.5 hr, diluted with EtOAc (80 ml), washed with water (2×20 ml), saturated aqueous NaHCO₃ (20 ml) and brine (20 ml), and dried over Na₂SO₄. Removal of the solvent left 1,2-dimethyl 4-(chlorosulfonyl)benzene-1,2-dicarboxylate (690 mg, 86%) as a yellow oil which was used without further purification. ¹H NMR (300 MHz, CHLOROFORM-d) 5: 8.45 (d, J=2.20 Hz, 1H), 8.14-8.27 (m, 1H), 7.89 (d, J=8.35 Hz, 1H), 3.96 (d, J=1.32 Hz, 6H). LC-MS (ESI+) t_{R} 4.62 min, m/z 261.

Step 2; To a stirred solution of 1,2-dimethyl 4-(chlorosulfonyl)benzene-1,2-dicarboxylate (CAS Regno: 1152495-27-6, 690 mg, 2.4 mmol) and 2-dimethyl 4-aminobenzene-1,2-dicarboxylate (CAS Regno: 51832-31-6, 500 mg, 2.4 mmol) in CH₂Cl₂ (15 ml) was added pyridine (5 ml). The mixture was stirred at rt for 4 days and concentrated. The residue was taken up in EtOAc (90 ml), washed with 5% aq HCl (2×15 ml) and brine (15 ml), and dried over Na₂SO₄. Removal of the solvent left an orange oil (1.04 g) which was chromatographed to give 1,2-dimethyl 4-{[3,4-bis(methoxycarbonyl)phenyl]sulfamoyl}benzene-1,2-dicarboxylate (698 mg, 62%). ¹H NMR (300 MHz, CHLOROFORM-d) δ: 8.23-8.27 (m, 1H), 8.04 (s, 1H), 7.90-7.96 (m, 1H), 7.72 (d, J=7.91 Hz, 1H), 7.68 (d, J=8.35 Hz, 1H), 7.39 (d, J=2.20 Hz, 1H), 7.29-7.35 (m, 1H), 3.91 (s, 3H), 3.90 (s, 3H), 3.87 (s, 3H), 3.85 (s, 3H). LC-MS (ESI+) t_{R} 4.30 min, m/z 434 (M-CH₃CO)⁺.

Step 3: To a stirred solution of 1,2-dimethyl 4-{[3,4-bis(methoxycarbonyl) phenyl]sulfamoyl}benzene-1,2-dicarboxylate (698 mg, 1.5 mmol) in DMF (7.5 ml) was added Cs₂CO₃ (587 mg, 1.8 mmol). The mixture was stirred for 10 min and Mel (CAS Regno: 74-88-4, 0.11 ml, 1.8 mmol) was added. The mixture was stirred overnight at rt, diluted with EtOAc (90 ml), washed with water (20 ml) and brine (20 ml), and dried over Na₂SO₄. Removal of the solvent left an oil (890 mg) which was chromatographed on a 40 g silica cartridge, eluted with a 0-80% EtOAc in hexanes gradient, to give 1,2-dimethyl 4-{[3,4-bis(methoxycarbonyl)phenyl](methyl)sulfamoyl}benzene-1,2-dicarboxylate (620 mg, 86%) as a colorless oil. ¹H NMR (300 MHz, CHLOROFORM-d) 5: 7.95-7.99 (m, 1H), 7.67-7.77 (m, 2H), 7.64 (d, J=1.76 Hz, 1H), 7.42 (d, J=2.20 Hz, 1H), 7.32-7.38 (m, 1H), 3.94 (s, 3H), 3.92 (s, 3H), 3.91 (s, 3H), 3.89 (s, 3H), 3.21 (s, 3H). LC-MS t_{R} 4.66, m/z 448 (M-CH₃CO)+.

Step 4: A solution of 1,2-dimethyl 4-{[3,4-bis(methoxycarbonyl)phenyl](methyl) sulfamoyl}benzene-1,2-dicarboxylate (620 mg, 1.33 mmol) in 3:1 MeOH/10% aq KOH (20 ml) was stirred at rt for 2 days and concentrated. The residue was taken up in water (25 ml) and washed with EtOAc (75 ml). The aq layer was acidified with conc HCl and extracted with EtOAc (2×50 ml). These EtOAc extracts were combined, washed with brine (10 ml), dried over Na₂SO₄ and concentrated to leave a dark oil (500 mg). This oil was combined with SOCl₂ (1 ml, 14.1 mmol), toluene (20 ml) and DMF (2 drops). The mixture was heated at reflux for 1 day and concentrated to leave crude N-(1,3-dioxo-1,3-dihydro-2-benzofuran-5-yl)-N-methyl-1,3-dioxo-1,3-dihydro-2-benzofuran-5-sulfonamide as a dark solid which was used directly in the next step.

Step 5: To a stirred mixture of crude N-(1,3-dioxo-1,3-dihydro-2-benzofuran-5-yl)-N-methyl-1,3-dioxo-1,3-dihydro-2-benzofuran-5-sulfonamide (51.3 mmol), 2,4-pentanedione (CAS Regno: 123-54-6, 300 mg, 2.9 mmol) and acetic anhydride (3 ml), Et₃N (1.5 ml, 10.7 mmol) was added dropwise over 1 min. The mixture was stirred at rt for 1.25 hr and 5% aq HCl (25 ml) was added. The mixture was stirred overnight and filtered. The solid collected was taken up in CH₂Cl₂ (80 ml), washed with 5% aq HCl (20 ml) and brine (20 ml), and dried over Na₂SO₄. Removal of the solvent left a brown solid (520 mg). A portion of the brown solid (390 mg) was taken up in CH₂Cl₂ (60 ml) and extracted with 1 M aq NaOH (20 ml). The aq NaOH layer was acidified with conc HCl and filtered. The solid collected was dried under vacuum (230 mg). A portion of this solid (50 mg) was purified by prep HPLC to give the title compound (2.8 mg, 0.5% over 2 steps) as a light brown solid. ¹H NMR (300 MHz, DMSO-d6) δ: 7.56-7.83 (m, 3H), 7.32-7.52 (m, 3H), 3.21 (s, 3H), 2.41 (s, 3H), 2.27 (m, 3H). LC-MS (ESI+) t_{R} 4.89 min, m/z 468. HRMS (ESI−): calculated for C₂₃H₁₇NOS m/z [M−H]⁻: 466.0602, observed 466.0608.

### Example 315—Synthesis of methyl (2S)-2-{[5-(2-{[(2S)-1-methoxy-1-oxopropan-2-yl]carbamoyl}-1,3-dioxo-2,3-dihydro-1H-indene-5-carbonyl)-1,3-dioxo-2,3-dihydro-1H-inden-2-yl]formamido}propanoate

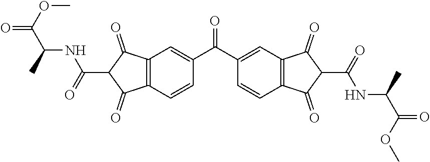 [SMILES: COC(=O)[C@H](C)NC(=O)C1C(=O)c2ccc(C(=O)c3ccc4c(c3)C(=O)C(C(=O)N[C@@H](C)C(=O)OC)C4=O)cc2C1=O]

A solution of 5,5′-carbonylbis(1H-indene-1,3(2H)-dione (Example 4, 157.6 mg, 0.495 mmol) in DMF was cooled to −40° C. in a dry ice MeCN bath and stirred for 5 min. To this was added dropwise, triethylamine (180 μl, 1.28 mmol) and the resulting mixture was stirred for 10 min. To this mixture was then added (S)-(−)-2-isocyanatopropionic acid methyl ester (CAS 30293-82-4, 150 μl, 1.30 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (261.7 mg, 85%). LCMS (ESI+): R_{f}=5.65, M+H=577.13; HRMS (ESI−): calculated for C₂₉H₂₃N₂O₁₁ m/z [M−H]⁻: 575.1307, observed 575.1321; ¹H NMR 300 MHz DMSO δ: 8.86-8.93 (m, 1H), 7.85-7.92 (m, 1H), 7.63-7.68 (m, 1H), 7.51-7.57 (m, 1H), 4.48-4.59 (m, 1H), 3.67-3.73 (m, 3H), 1.37-1.45 (m, 3H).

### Example 316—Synthesis of (2S)-2-{[5-(2-{[(1S)-1-carboxyethyl]carbamoyl}-1,3-dioxo-2,3-dihydro-1H-indene-5-carbonyl)-1,3-dioxo-2,3-dihydro-1H-inden-2-yl]formamido}propanoic acid

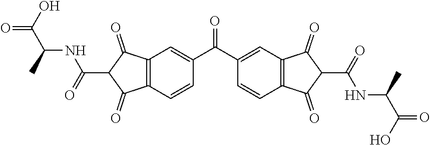 [SMILES: C[C@H](NC(=O)C1C(=O)c2ccc(C(=O)c3ccc4c(c3)C(=O)C(C(=O)N[C@@H](C)C(=O)O)C4=O)cc2C1=O)C(=O)O]

To a 40 ml vial equipped with a stirbar was charged methyl (2S)-2-{[5-(2-{[(2S)-1-methoxy-1-oxopropan-2-yl]carbamoyl}-1,3-dioxo-2,3-dihydro-1H-indene-5-carbonyl)-1,3-dioxo-2,3-dihydro-1H-inden-2-yl]formamido}propanoate (Example 315, 189.9 mg, 0.306 mmol) and 5 ml of MeOH to produce a dark slurry. To this was added 5 ml of 1N NaOH and the resulting mixture was allowed to stir overnight at ambient temperature. Once the reaction was deemed complete by LCMS, the reaction was transferred to a water bath and 5 ml of 2N HCl was added dropwise, causing a precipitate to form. The slurry was stirred for 5 min, then the solid was isolated by filtration. The solid was washed with 10 ml of H₂O, then dried in the vacuum oven at 45° C. and full house vacuum to provide the title compound as the dark brown free acid (112.8 mg, 67%). LCMS (ESI+): R_{f}=4.46, M+H=5549.11; HRMS (ESI−): calculated for C₂₇H₁₉N₂O₁₁ m/z [M−H]⁻: 547.0994, observed 547.1007; ¹H NMR 300 MHz DMSO δ: 7.99-8.05 (m, 1H), 7.84 (s, 1H), 7.75 (d, J=7.62 Hz, 1H), 4.52-4.62 (m, 2H), 1.45-1.55 (m, 3H).

### Example 317—Synthesis of methyl (2S)-2-({5-[(2-{[(2S)-1-methoxy-1-oxopropan-2-yl]carbamoyl}-1,3-dioxo-2,3-dihydro-1H-inden-5-yl)sulfonyl]-1,3-dioxo-2,3-dihydro-1H-inden-2-yl}formamido)propanoate

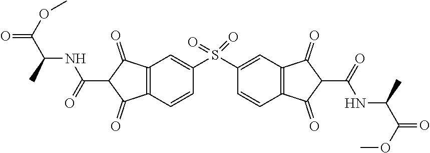 [SMILES: COC(=O)[C@H](C)NC(=O)C1C(=O)c2ccc(S(=O)(=O)c3ccc4c(c3)C(=O)C(C(=O)N[C@@H](C)C(=O)OC)C4=O)cc2C1=O]

A solution of 5,5′-sulfonylbis(1H-indene-1,3(2H)-dione) (Example 11, 171 mg, 0.497 mmol) in DMF was cooled to −40° C. in a dry ice MeCN bath and stirred for 5 min. To this was added dropwise, triethylamine (180 μl, 1.28 mmol) and the resulting mixture was stirred for 10 min. To this was then added (S)-(−)-2-isocyanatopropionic acid methyl ester (CAS 30293-82-4, 150 μl, 1.30 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (275.8 mg, 85%). LCMS (ESI+): R_{f}=5.11, M+H=613.06; HRMS (ESI−): calculated for C₂₈H₂₃N₂O₁₂S m/z [M−H]⁻: 611.0977, observed 611.0972; ¹H NMR 300 MHz DMSO δ: 8.79-8.86 (m, 1H), 8.11-8.19 (m, 1H), 7.73-7.78 (m, 1H), 7.56-7.62 (m, 1H), 4.46-4.58 (m, 1H), 3.68-3.74 (m, 3H), 1.37-1.47 (m, 3H).

### Example 318—Synthesis of (2S)-2-({5-[(2-{[(1S)-1-carboxyethyl]carbamoyl}-1,3-dioxo-2,3-dihydro-1H-inden-5-yl)sulfonyl]-1,3-dioxo-2,3-dihydro-1H-inden-2-yl}formamido) propanoic acid

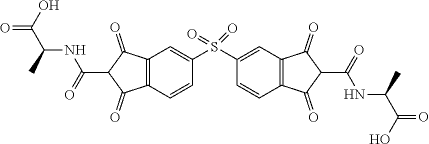 [SMILES: C[C@H](NC(=O)C1C(=O)c2ccc(S(=O)(=O)c3ccc4c(c3)C(=O)C(C(=O)N[C@@H](C)C(=O)O)C4=O)cc2C1=O)C(=O)O]

To a 40 ml vial equipped with a stirbar was charged of methyl (2S)-2-({5-[(2-{[(2S)-1-methoxy-1-oxopropan-2-yl]carbamoyl}-1,3-dioxo-2,3-dihydro-1H-inden-5-yl)sulfonyl]-1,3-dioxo-2,3-dihydro-1H-inden-2-yl}formamido)propanoate (Example 317, 185.7 mg, 0.282 mmol) and 5 ml of MeOH to produce a dark slurry. To this was added 5 ml of 1N NaOH and the resulting mixture was allowed to stir overnight at ambient temperature. Once the reaction was deemed complete by LCMS, the reaction was transferred to a water bath and 5 ml of 2N HCl was added dropwise, causing a precipitate to form. The slurry was stirred for 5 min, then the solid was isolated by filtration. The solid was washed with 10 ml of H₂O, then dried in the vacuum oven at 45° C. and full house vacuum to provide the title compound as the dark brown free acid (157.7 mg, 95%). LCMS (ESI+): R_{f}=3.93, M+H=585.03; HRMS (ESI−): calculated for C₂₆H₁₉N₂O₁₂S m/z [M−H]⁻: 583.0664, observed 583.0655; ¹H NMR 300 MHz DMSO δ: 8.26 (dd, J=1.46, 7.91 Hz, 1H), 7.89-7.93 (s, 1H), 7.67-7.74 (m, 1H), 4.46-4.56 (m, 2H), 1.41-1.52 (m, 3H).

### Example 319—Synthesis of tert-butyl N-[2-(5-{2-[2-(2-{[(tert-butoxy)carbonyl]amino}acetyl)-1,3-dioxo-2,3-dihydro-1H-inden-5-yl]ethynyl}-1,3-dioxo-2,3-dihydro-1H-inden-2-yl)-2-oxoethyl]carbamate

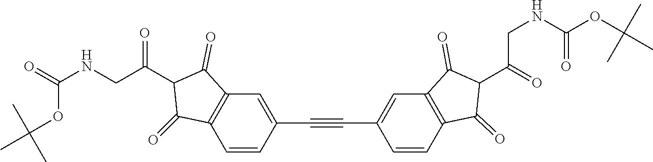 [SMILES: CC(C)(C)OC(=O)NCC(=O)C1C(=O)c2ccc(C#Cc3ccc4c(c3)C(=O)C(C(=O)CNC(=O)OC(C)(C)C)C4=O)cc2C1=O]

To a stirred solution of Boc-glycine (CAS Regno: 4530-20-5, 502 mg, 2.9 mmol) and DMAP (350 mg, 2.9 mmol) in dry DMF (10 ml) was added EDC·HCl (550 mg, 2.9 mmol). The mixture was stirred at rt for 5 min and 5-[2-(1,3-dioxo-2,3-dihydro-1H-inden-5-yl)ethynyl]-2,3-dihydro-1H-indene-1,3-dione (Example 196, 300 mg, 1.0 mmol) was added. The mixture was stirred overnight at rt and diluted with water (70 ml). After stirring for 30 min, the mixture was filtered. The dark brown solid collected (450 mg) was stirred with ice-cold 5% aq HCl (25 ml) and CH₂Cl₂ (25 ml) for 15 min and filtered. The brown solid collected (300 mg) was sonicated with 1:1 CH₂Cl₂/MeOH (2×20 ml) and with MeOH (20 ml). The soluble fractions were combined and concentrated to give a rust brown solid (21 mg). This solid was triturated with CH₂Cl₂ (10 ml) and 1:1 CH₂Cl₂/MeOH (20 ml). The soluble fraction from the 1:1 CH₂Cl₂/MeOH trituration was concentrated to provide the title compound (8.4 mg, 1.4%) as a solid. ¹H NMR (300 MHz, DMSO-d6) δ: 7.61-7.71 (m, 2H), 7.45-7.50 (m, 2H), 7.35-7.43 (m, 2H), 6.17-6.31 (m, 2H), 5.61-5.73 (m, 2H), 4.08-4.17 (m, 4H), 1.40 (s, 18H). LC-MS (ESI+) t_{R} 5.52 min, m/z 629, 473. HRMS (ESI−): calculated for C₃₄H₃₁N₂O₁₀ m/z [M−H]⁻: 627.1984, observed 627.1987.

### Example 320—Synthesis of tert-butyl N-[2-(5-{2-[2-(2-{[(tert-butoxy)carbonyl](methyl)amino}acetyl)-1,3-dioxo-2,3-dihydro-1H-inden-5-yl]ethynyl}-1,3-dioxo-2,3-dihydro-1H-inden-2-yl)-2-oxoethyl]-N-methylcarbamate

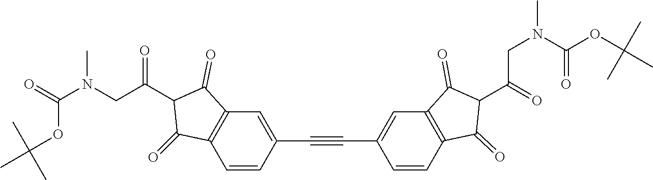 [SMILES: CN(CC(=O)C1C(=O)c2ccc(C#Cc3ccc4c(c3)C(=O)C(C(=O)CN(C)C(=O)OC(C)(C)C)C4=O)cc2C1=O)C(=O)OC(C)(C)C]

To a stirred solution of Boc-sarcosine (CAS Regno: 13734-36-6, 542 mg, 2.9 mmol) and DMAP (350 mg, 2.9 mmol) in dry DMF (10 ml) was added EDC·HCl (550 mg, 2.9 mmol). The mixture was stirred at rt for 10 min and 5-[2-(1,3-dioxo-2,3-dihydro-1H-inden-5-yl)ethynyl]-2,3-dihydro-1H-indene-1,3-dione (Example 196, 300 mg, 1.0 mmol) was added. The mixture was stirred overnight at rt, diluted with water (20 ml), stirred for 15 min and filtered. The dark solid collected was stirred with water (25 ml) and CH₂Cl₂ (25 ml) for 15 min, treated with 5% aq HCl (5 ml) and filtered. The CH₂Cl₂ layer of the filtrate was separated, diluted with additional CH₂Cl₂ (35 ml), washed with brine (20 ml) and dried over Na₂SO₄. Removal of the solvent left a brown solid (180 mg) which was sonicated with water (7 ml) and centrifuged. The supernatant was removed. The process of sonication, centrifugation and removal of the supernatant was repeated with, saturated aqueous NaHCO₃ (7 ml) and water (7 ml) to leave the title compound as a dark solid (58 mg, 9%). ¹H NMR (300 MHz, DMSO-d6) 7.63-7.70 (m, 2H), 7.48 (s, 2H), 7.36-7.43 (m, 2H), 4.33 (s, 4H), 2.78 (s, 3H), 2.68 (s, 3H), 1.37 (s, 9H), 1.28 (s, 9H). LC-MS (ESI+) t_{R} 6.35 min, m/z 657, 557, 501. HRMS (ESI−): calculated for C₃₆H₃₅N₂O₁₀ m/z [M−H]⁻: 655.2297, observed 655.2291.

### Example 321—Synthesis of 2-butanoyl-5-[(2-butanoyl-1,3-dioxo-2,3-dihydro-1H-inden-5-yl)oxy]-2,3-dihydro-1H-indene-1,3-dione

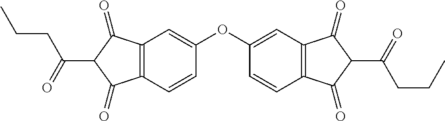 [SMILES: CCCC(=O)C1C(=O)c2ccc(Oc3ccc4c(c3)C(=O)C(C(=O)CCC)C4=O)cc2C1=O]

To a solution of dimethylaminopyridine (DMAP, 183 mg, 1.50 mmol), butyric acid (CAS number 107-92-6, 91.8 mg, 2.03 mmol) in anhydrous DMF (10 ml) was added EDCI HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 157.1 mg, 0.513 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water, then dried in a vacuum oven. The dried solid was milled into a fine powder and was treated with 10 ml of 1M NaOH and 5 ml of methylene chloride. This biphasic suspension was shaken vigorously and filtered to provide the title compound as the dark solid bis-sodium salt (163.7 mg, 65%). LCMS (ESI+): R_{f}=7.38, M+H=447.08; HRMS (ESI−): calculated for C₂₆H₂₁O₇ m/z [M−H]⁻: 445.1293, observed 445.1284; ¹H NMR 300 MHz DMSO δ: 7.34-7.38 (m, 1H), 7.05-7.10 (m, 1H), 6.86-6.89 (m, 1H), 2.62-2.69 (m, 2H), 1.41-1.54 (m, 2H), 0.81-0.90 (m, 3H).

### Example 322—Synthesis of 2-butanoyl-5-[(2-butanoyl-1,3-dioxo-2,3-dihydro-1H-inden-5-yl)sulfonyl]-2,3-dihydro-1H-indene-1,3-dione

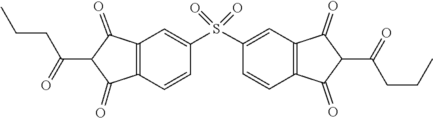 [SMILES: CCCC(=O)C1C(=O)c2ccc(S(=O)(=O)c3ccc4c(c3)C(=O)C(C(=O)CCC)C4=O)cc2C1=O]

To a solution of dimethylaminopyridine (DMAP, 183 mg, 1.50 mmol), butyric acid (CAS number 107-92-6, 104.9 mg, 1.19 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 11, 184 mg, 0.521 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water, then dried in a vacuum oven. The dried solid was milled into a fine powder and was treated with 10 ml of 1M NaOH and 5 ml of methylene chloride. This biphasic suspension was shaken vigorously and filtered to provide the title compound as the dark solid bis-sodium salt (163.7 mg, 77%). LCMS (ESI+): R_{f}=6.64, M+H=495.03; HRMS (ESI−): calculated for C₂₆H₂₁O₈S m/z [M−H]⁻: 493.0963, observed 493.0974; ¹H NMR 300 MHz DMSO δ: 8.02-8.11 (m, 1H), 7.68-7.72 (m, 1H), 7.49-7.55 (m, 1H), 2.61-2.69 (m, 2H), 1.41-1.50 (m, 2H), 0.80-0.87 (m, 3H).

### Example 323—Synthesis of 5-[(1,3-dioxo-2-propanoyl-2,3-dihydro-1H-inden-5-yl)oxy]-2-propanoyl-2,3-dihydro-1H-indene-1,3-dione

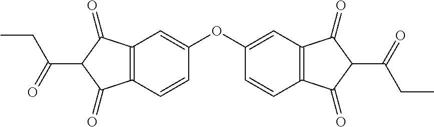 [SMILES: CCC(=O)C1C(=O)c2ccc(Oc3ccc4c(c3)C(=O)C(C(=O)CC)C4=O)cc2C1=O]

To a solution of dimethylaminopyridine (DMAP, 183 mg, 1.50 mmol), propionic acid (CAS number 72-09-4, 145.8 mg, 1.97 mmol) in anhydrous DMF (10 ml) was added EDCI HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 152.4 mg, 0.497 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water, then dried in a vacuum oven. The dried solid was milled into a fine powder and was treated with 10 ml of 1M NaOH and 5 ml of methylene chloride. This biphasic suspension was shaken vigorously and filtered to provide the title compound as the dark solid bis-sodium salt (111.7 mg, 49%). LCMS (ESI+): R_{f}=6.74, M+H=419.00; HRMS (ESI−): calculated for C₂₄H₁₇O₇ m/z [M−H]⁻: 417.0980, observed 417.0983; ¹H NMR 300 MHz DMSO δ: 7.35-7.40 (m, 1H), 7.08 (dd, J=2.34, 8.20 Hz, 1H), 6.87-6.89 (m, 1H), 2.64-2.74 (m, 2H), 0.90-0.99 (m, 3H).

### Example 324—Synthesis of 5-(1,3-dioxo-2-propanoyl-2,3-dihydro-1H-indene-5-carbonyl)-2-propanoyl-2,3-dihydro-1H-indene-1,3-dione

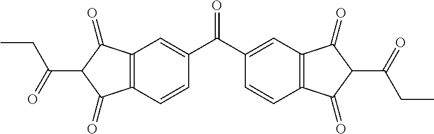 [SMILES: CCC(=O)C1C(=O)c2ccc(C(=O)c3ccc4c(c3)C(=O)C(C(=O)CC)C4=O)cc2C1=O]

To a solution of dimethylaminopyridine (DMAP, 183 mg, 1.50 mmol), propionic acid (CAS number 72-09-4, 137.6 mg, 1.86 mmol) in anhydrous DMF (10 ml) was added EDCI·HCl (286.5 mg, 1.50 mmol) and the contents were stirred for 10 min. 5,5′-carbonylbis(1H-indene-1,3(2H)-dione (Example 4, 156 mg, 0.490 mmol) was added and the reaction solution was stirred ambient temperature. Once the reaction was deemed complete by LCMS, 15 ml of 2N HCl was added and the resulting precipitant was filtered. The solid was washed once with 5 ml of 2N HCl and 5 ml of water. The solid was dried overnight in the vacuum oven to provide a tan solid. The dried solid was milled into a fine powder and was treated with 10 ml of 1N NaOH and 10 ml of methylene chloride. This biphasic suspension was shaken vigorously and filtered to provide the title compound as the dark solid bis-sodium salt (203.2 mg, 87%). LCMS (ESI+): R_{f}=6.44, M+H=431.04; HRMS (ESI−): calculated for C₂₅H₁₇O₇ m/z [M−H]⁻: Exact Mass: 429.0980, observed 429.0990; ¹H NMR 300 MHz DMSO δ: 7.81-7.86 (m, 1H), 7.59-7.63 (m, 1H), 7.49-7.54 (m, 1H), 2.68-2.78 (m, 2H), 0.91-0.99 (m, 3H).

### Example 325—Synthesis of 5-[(1,3-dioxo-2-propanoyl-2,3-dihydro-1H-inden-5-yl)sulfonyl]-2-propanoyl-2,3-dihydro-1H-indene-1,3-dione

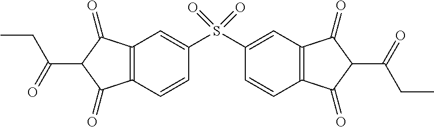 [SMILES: CCC(=O)C1C(=O)c2ccc(S(=O)(=O)c3ccc4c(c3)C(=O)C(C(=O)CC)C4=O)cc2C1=O]

To a solution of dimethylaminopyridine (DMAP, 183 mg, 1.50 mmol), propionic acid (CAS number 72-09-4, 107.6 mg, 1.45 mmol) in anhydrous DMF (10 ml) was added EDCI HCl (286.5 mg, 1.50 mmol) and the contents were stirred for 10 min. 5,5′-sulfonylbis(1H-indene-1,3(2H)-dione) (Example 11, 190.6 mg, 0.538 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water, then dried in a vacuum oven. The dried solid was milled into a fine powder and was treated with 10 ml of 1M NaOH and 5 ml of methylene chloride. This biphasic suspension was shaken vigorously and filtered to provide the title compound as the dark solid bis-sodium salt (155.2 mg, 56.5%). LCMS (ESI+): R_{f}=6.26, M+H=peak failed to ionize; HRMS (ESI−): calculated for C₂₄H₁₇O₈S m/z [M−H]⁻: 465.0650, observed 465.0655; ¹H NMR 300 MHz DMSO δ: 8.04-8.09 (m, 1H), 7.71 (d, J=1.17 Hz, 1H), 7.50-7.56 (m, 1H), 2.63-2.74 (m, 2H), 0.88-0.97 (m, 3H).

### Example 326—Synthesis of 2-(2-aminoacetyl)-5-{[2-(2-aminoacetyl)-1,3-dioxo-2,3-dihydro-1H-inden-5-yl]oxy}-2,3-dihydro-1H-indene-1,3-dione

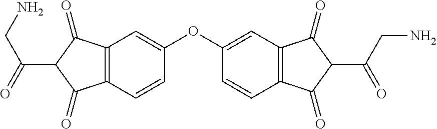 [SMILES: NCC(=O)C1C(=O)c2ccc(Oc3ccc4c(c3)C(=O)C(C(=O)CN)C4=O)cc2C1=O]

To a slurry of tert-butyl N-[2-(5-{[2-(2-{[(tert-butoxy)carbonyl]amino}acetyl)-1,3-dioxo-2,3-dihydro-1H-inden-5-yl]oxy}-1,3-dioxo-2,3-dihydro-1H-inden-2-yl)-2-oxoethyl]carbamate, bis sodium salt (Example 272, 52.6 mg, 0.079 mmol) in 5 ml of DCM was charged 1.0 ml of trifluoroacetic acid (TFA, 1 ml, 13.06 mmol). The addition of the TFA produced a dark brown solution. The reaction was stirred at ambient temperature. Once the reaction was deemed complete by LCMS, 2.5 ml of H₂O was added and the resulting precipitant was filtered. The solid was washed with 5 ml of DCM. The tan solid was dried overnight in the vacuum oven. The title compound was obtained as the bis trifluoroacetic acid salt (32.6 mg, 41% yield). LCMS (ESI+): R_{f}=2.67, M+H=421.11; ¹H NMR 300 MHz DMSO δ: 7.65-7.77 (m, 4H), 7.42-7.47 (m, 1H), 7.11-7.17 (m, 1H), 6.91-6.96 (m, 1H), 3.91-4.00 (m, 3H).

### Example 327—Synthesis of 2-(2-aminoacetyl)-5-[2-(2-aminoacetyl)-1,3-dioxo-2,3-dihydro-1H-indene-5-carbonyl]-2,3-dihydro-1H-indene-1,3-dione

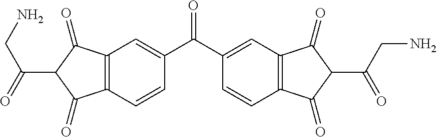 [SMILES: NCC(=O)C1C(=O)c2ccc(C(=O)c3ccc4c(c3)C(=O)C(C(=O)CN)C4=O)cc2C1=O]

To a slurry of tert-butyl N-(2-{5-[2-(2-{[(tert-butoxy)carbonyl]amino}acetyl)-1,3-dioxo-2,3-dihydro-1H-indene-5-carbonyl]-1,3-dioxo-2,3-dihydro-1H-inden-2-yl}-2-oxoethyl) carbamate bis sodium salt (Example 173, 53.9 mg, 0.080 mmol) in 5 ml of DCM was charged 1 ml of trifluoroacetic acid (TFA, 1.0 ml, 13.06 mmol). The addition of the TFA produced a dark brown solution. The reaction was stirred at ambient temperature. Once the reaction was deemed complete by LCMS, 2.5 ml of H₂O was added and the resulting precipitant was filtered. The solid was washed with 5 ml of DCM. The tan solid was dried overnight in the vacuum oven. The title compound was obtained as the bis trifluoroacetic acid salt (22.8 mg, 46% yield). LCMS (ESI+): R_{f}=2.43, M+H=433.28; HRMS (ESI−): calculated for C₂₃H₁₅N₂O₇ m/z [M−H]⁻: Exact Mass: 431.088474, observed 431.088341; ¹H NMR 300 MHz DMSO δ: 7.85-7.90 (m, 1H), 7.72 (t, J=4.98 Hz, 3H), 7.66 (d, J=1.17 Hz, 1H), 7.55-7.60 (m, 1H), 3.96-4.04 (m, 2H).

### Example 328—Synthesis of N-cyclopropyl-5-[2-(cyclopropylcarbamothioyl)-1,3-dioxo-2,3-dihydro-1H-indene-5-carbonyl]-1,3-dioxo-2,3-dihydro-1H-indene-2-carbothioamide

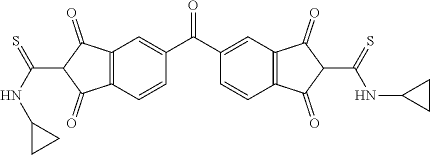 [SMILES: O=C(c1ccc2c(c1)C(=O)C(C(=S)NC1CC1)C2=O)c1ccc2c(c1)C(=O)C(C(=S)NC1CC1)C2=O]

A solution of 5,5′-carbonylbis(1H-indene-1,3(2H)-dione (Example 4, 159 mg, 0.500 mmol) in DMF was cooled to −40° C. in a dry ice MeCN bath and stirred for 5 min. To this was added dropwise, triethylamine (180 μl, 1.28 mmol) and the resulting mixture was stirred for 10 min. To this mixture was then added cyclopropyl thioisocyanate (CAS 56601-42-4, 120 μl, 1.32 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (135.8 mg, 48%). LCMS (ESI+): R_{f}=7.31, M+H=517.62; HRMS (ESI−): calculated for C₂₇H₁₉N₂O₅S₂ m/z [M−H]⁻: 515.074087, observed 515.072825; ¹H NMR 300 MHz DMSO δ: 10.11-10.22 (m, 1H), 7.87-8.03 (m, 2H), 7.62-7.82 (m, 3H), 3.00-3.16 (m, 1H), 0.85-0.98 (m, 2H), 0.70-0.85 (m, 2H).

### Example 329—Synthesis of N-cyclopropyl-5-{[2-(cyclopropylcarbamothioyl)-1,3-dioxo-2,3-dihydro-1H-inden-5-yl]sulfonyl}-1,3-dioxo-2,3-dihydro-1H-indene-2-carbothioamide

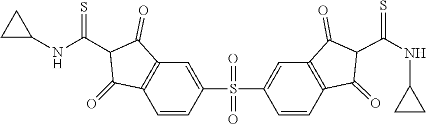 [SMILES: O=C1c2ccc(S(=O)(=O)c3ccc4c(c3)C(=O)C(C(=S)NC3CC3)C4=O)cc2C(=O)C1C(=S)NC1CC1]

A solution of 5,5′-sulfonylbis(1H-indene-1,3(2H)-dione) (Example 11, 177 mg, 0.500 mmol) in DMF was cooled to −40° C. in a dry ice MeCN bath and stirred for 5 min. To this was added dropwise, triethylamine (180 μl, 1.28 mmol) and the resulting mixture was stirred for 10 min. To this mixture was then added cyclopropyl thioisocyanate (CAS 56601-42-4, 120 μl, 1.32 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (155.7 mg, 56%). LCMS (ESI+): R_{f}=6.69, M+H=553.60; HRMS (ESI−): calculated for C₂₆H₁₉N₂O₈S₃ m/z [M−H]⁻: 551.041073, observed 551.0398766; ¹H NMR 300 MHz DMSO δ: 10.75-10.84 (m, 1H), 7.99-8.07 (m, 1H), 7.63-7.67 (m, 1H), 7.43-7.51 (m, 1H), 0.67-0.79 (m, 2H), 0.41-0.53 (m, 2H).

### Example 330—Synthesis of 6-acetyl-2-[(2-acetyl-1,3-dioxo-2,3-dihydro-1H-inden-5-yl)oxy]-5H,6H,7H-cyclopenta[b]pyridine-5,7-dione

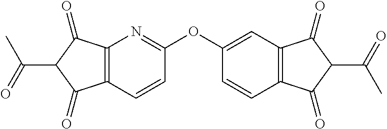 [SMILES: CC(=O)C1C(=O)c2ccc(Oc3ccc4c(n3)C(=O)C(C(C)=O)C4=O)cc2C1=O]

Step 1: To a stirred solution of 1,2-dimethyl 4-hydroxybenzene-1,2-dicarboxylate (CAS Regno: 22479-95-4, 200 mg, 0.95 mmol) in dry THF (2 ml) cooled to −70° C. was added dropwise 1 M KN(SiMe₃)₂ in THF (1 ml, 1.0 mmol). The mixture was stirred at −70° C. for 5 min and allowed to warm to rt. 2,3-Dimethyl 6-chloropyridine-2,3-dicarboxylate (CAS Regno: 32383-03-2, 220 mg, 0.95 mmol) was added, followed by dry DMF (2 ml). The mixture was stirred at rt for 1 day, diluted with EtOAc (90 ml), washed with 5% aq HCl (20 ml), 1 M aq NaOH (20 ml) and brine (20 ml), and dried over Na₂SO₄. Removal of the solvent left an oil (330 mg), which was chromatographed on a 40 g silica cartridge, eluted with a 0-50% EtOAc in hexanes gradient to give 2,3-dimethyl 6-[3,4-bis(methoxycarbonyl)phenoxy]pyridine-2,3-dicarboxylate (126 mg, 33%) as an oil. ¹H NMR (300 MHz, CHLOROFORM-d) δ 8.17-8.38 (m, 1H), 7.79 (d, J=8.35 Hz, 1H), 7.46 (d, J=2.64 Hz, 1H), 7.29-7.37 (m, 1H), 7.05 (d, J=8.35 Hz, 1H), 3.90 (s, 6H), 3.89 (s, 6H). LC-MS ESI⁺ t_{R} 4.43 min, m/z 426, 404, 372.

Step 2: A solution of 2,3-dimethyl 6-[3,4-bis(methoxycarbonyl)phenoxy]pyridine-2,3-dicarboxylate (126 mg, 0.31 mmol) in MeOH (9 ml) and 1 M aq NaOH (3 ml, 3.0 mmol) was stirred at rt for 3 day. Additional MeOH (3 ml) and 1 M aq NaOH (1.5 ml, 1.5 mmol) were added and stirring at rt was continued for 4 days. The mixture was concentrated under reduced pressure. The solid residue was taken up in 5% aq HCl (5 ml) and MeCN (5 ml) and concentrated to dryness under reduced pressure to leave crude 6-(3,4-dicarboxyphenoxy)pyridine-2,3-dicarboxylic acid which was used directly in the next step. LC-MS ESI⁺ t_{R} 2.63 min, m/z 330, 312.

Step 3: Crude 6-(3,4-dicarboxyphenoxy)pyridine-2,3-dicarboxylic acid (50.31 mmol) from the previous step was heated at reflux with toluene (6 ml) and SOCl₂ (2.7 ml) for 18 hr. The mixture was concentrated, chasing with CH₂Cl₂, to give crude 2-[(1,3-dioxo-1,3-dihydro-2-benzofuran-5-yl)oxy]-5H,7H-furo[3,4-b]pyridine-5,7-dione which was used directly in the next step.

Step 4: To a stirred mixture of crude 2-[(1,3-dioxo-1,3-dihydro-2-benzofuran-5-yl)oxy]-5H,7H-furo[3,4-b]pyridine-5,7-dione (50.31 mmol) from the previous step, pentane-2,4-dione (80 mg, 0.79 mmol) and acetic anhydride (1.4 ml, 14.8 mmol) was added Et₃N (0.4 ml, 2.8 mmol). The mixture was stirred at rt for 1 hr and 5% aq HCl (5 ml) was added. The mixture was stirred at rt for 2 hr, diluted with water (5 ml) and extracted with CH₂Cl₂ (45 ml). The organic layer was washed with brine (5 ml), dried over Na₂SO₄ and concentrated to leave an oil (200 mg). Chromatography on a silica cartridge, eluted with a 0-100% EtOAc in hexanes gradient, gave two fractions: 6,6-diacetyl-2-[(2,2-diacetyl-1,3-dioxo-2,3-dihydro-1H-inden-5-yl)oxy]-5H,6H,7H-cyclopenta[b]pyridine-5,7-dione (32 mg, eluted first) and 6,6-diacetyl-2-[(2-acetyl-1,3-dioxo-2,3-dihydro-1H-inden-5-yl)oxy]-5H,6H,7H-cyclopenta[b]pyridine-5,7-dione (60 mg, eluted second). Each fraction was separately taken up in acetic anhydride (1.5 ml) and treated with Et₃N (0.15 ml). The mixtures were each stirred overnight at rt, diluted with water (5 ml) and extracted with CH₂Cl₂ (2×35 ml). The combined CH₂Cl₂ extracts from each reaction were washed with brine (10 ml), dried over Na₂SO₄ and concentrated to give two crude products (60 mg from each) which both contained desired product. Prep HPLC gave the title compound (7.6 mg, 6% from Step 1 product) as an ochre yellow solid. ¹H NMR (300 MHz, DMSO-d6) δ 7.92-8.07 (m, 1H), 7.72 (br d, J=8.35 Hz, 1H), 7.42-7.51 (m, 1H), 7.39 (br s, 1H), 7.19-7.28 (m, 1H), 2.43 (s, 3H), 2.32 (s, 3H). LC-MS ESI⁺ t_{R} 4.72 min, m/z 392. HRMS (ESI−): calculated for C₂₁H₁₃NO₇ m/z [M−H]⁻: 390.061925, observed 390.061298.

### Example 331—Synthesis of methyl (2S,3S)-2-({5-[(2-{[(2S,3S)-1-methoxy-3-methyl-1-oxopentan-2-yl]carbamoyl}-1,3-dioxo-2,3-dihydro-1H-inden-5-yl)oxy]-1,3-dioxo-2,3-dihydro-1H-inden-2-yl}formamido)-3-methylpentanoate

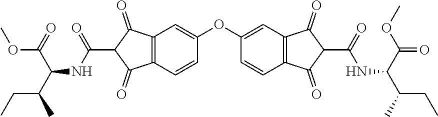 [SMILES: CC[C@H](C)[C@H](NC(=O)C1C(=O)c2ccc(Oc3ccc4c(c3)C(=O)C(C(=O)N[C@H](C(=O)OC)[C@@H](C)CC)C4=O)cc2C1=O)C(=O)OC]

A solution of 5-(2,3-dihydro-1,3-dioxo-1H-inden-6-yloxy)-2,3-dihydro-1,3-dioxo-1H-indene (Example 7, 155 mg, 0.500 mmol) in DMF was cooled to −40° C. in a dry ice MeCN bath and stirred for 5 min. To this was added dropwise, triethylamine (180 μl, 1.28 mmol) and the resulting mixture was stirred for 10 min. To this mixture was then added pentanoic acid, 2-isocyanato-3-methyl-,methyl ester, (2S,3S)- (CAS 120219-17-2, 200 μl, 1.31 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (276 mg, 78%). LCMS (ESI+): R_{f}=7.29, M+H=649.66; HRMS (ESI−): calculated for C₃₄H₃₅N₂O₁₁ m/z [M−H]⁻: 647.224634, observed 647.223650; ¹H NMR 300 MHz DMSO δ: 8.88-8.95 (m, 1H), 7.28-7.33 (m, 1H), 7.01-7.06 (m, 1H), 6.84-6.88 (m, 1H), 4.37-4.47 (m, 1H), 3.56-3.63 (m, 3H), 1.73-1.87 (m, 1H), 1.33-1.50 (m, 2H), 1.15-1.26 (m, 2H), 0.80-0.93 (m, 8H).

### Example 332—Synthesis of methyl (2S,3S)-2-{[5-(2-{[(2S,3S)-1-methoxy-3-methyl-1-oxopentan-2-yl]carbamoyl}-1,3-dioxo-2,3-dihydro-1H-indene-5-carbonyl)-1,3-dioxo-2,3-dihydro-1H-inden-2-yl]formamido}-3-methylpentanoate

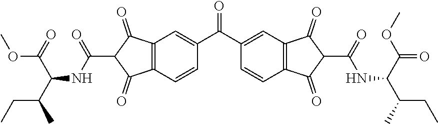 [SMILES: CC[C@H](C)[C@H](NC(=O)C1C(=O)c2ccc(C(=O)c3ccc4c(c3)C(=O)C(C(=O)N[C@H](C(=O)OC)[C@@H](C)CC)C4=O)cc2C1=O)C(=O)OC]

A solution of 5,5′-carbonylbis(1H-indene-1,3(2H)-dione (Example 4, 159 mg, 0.500 mmol) in DMF was cooled to −40° C. in a dry ice MeCN bath and stirred for 5 min. To this was added dropwise, triethylamine (180 μl, 1.28 mmol) and the resulting mixture was stirred for 10 min. To this mixture was then added pentanoic acid, 2-isocyanato-3-methyl-,methyl ester, (2S,3S)- (CAS 120219-17-2, 200 μl, 1.31 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (302.7 mg, 86%). LCMS (ESI+): R_{f}=7.15, M+H=661.68; HRMS (ESI−): calculated for C₃₅H₃₅N₂O₁₁ m/z [M−H]⁻: 659.224634, observed 659.225108; ¹H NMR 300 MHz DMSO δ: 8.92-8.99 (m, 1H), 7.79-7.84 (m, 1H), 7.54-7.59 (m, 1H), 7.43-7.49 (m, 1H), 4.39-4.48 (m, 1H), 3.58-3.65 (M, 3H), 1.75-1.89 (m, 1H), 1.37-1.50 (m, 1H), 1.11-1.27 (m, 2H), 0.82-0.93 (m, 6H).

### Example 333—Synthesis of methyl (2S,3S)-2-({5-[(2-{[(2S,3S)-1-methoxy-3-methyl-1-oxopentan-2-yl]carbamoyl}-1,3-dioxo-2,3-dihydro-1H-inden-5-yl)sulfonyl]-1,3-dioxo-2,3-dihydro-1H-inden-2-yl}formamido)-3-methylpentanoate

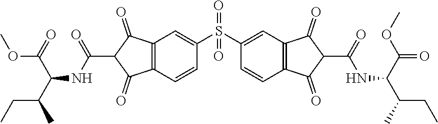 [SMILES: CC[C@H](C)[C@H](NC(=O)C1C(=O)c2ccc(S(=O)(=O)c3ccc4c(c3)C(=O)C(C(=O)N[C@H](C(=O)OC)[C@@H](C)CC)C4=O)cc2C1=O)C(=O)OC]

A solution of 5,5′-sulfonylbis(1H-indene-1,3(2H)-dione) (Example 11, 177 mg, 0.500 mmol) in DMF was cooled to −40° C. in a dry ice MeCN bath and stirred for 5 min. To this was added dropwise, triethylamine (180 μl, 1.28 mmol) and the resulting mixture was stirred for 10 min. To this mixture was then added pentanoic acid, 2-isocyanato-3-methyl-,methyl ester, (2S,3S)- (CAS 120219-17-2, 120 μl, 1.32 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (310.8 mg, 56%). LCMS (ESI+): R_{f}=6.78, M+H=697.59; HRMS (ESI−): calculated for C₃₄H₃₅N₂O₁₂S m/z [M−H]⁻: 695.191619, observed 695.189666; ¹H NMR 300 MHz DMSO δ: 8.83-8.91 (m, 1H), 8.03-8.10 (m, 1H), 7.63-7.69 (m, 1H), 7.46-7.52 (m, 1H), 4.37-4.46 (m, 1H), 3.56-3.64 (m, 3H), 1.72-1.87 (m, 1H), 1.32-1.48 (m, 1H), 1.10-1.25 (m, 1H), 0.79-0.91 (m, 6H).

### Example 334—Synthesis of N-ethyl-5-{[2-(ethylcarbamoyl)-1,3-dioxo-2,3-dihydro-1H-inden-5-yl]oxy}-1,3-dioxo-2,3-dihydro-1H-indene-2-carboxamide

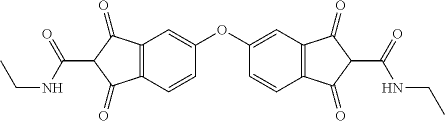 [SMILES: CCNC(=O)C1C(=O)c2ccc(Oc3ccc4c(c3)C(=O)C(C(=O)NCC)C4=O)cc2C1=O]

A solution of 5-(2,3-dihydro-1,3-dioxo-1H-inden-6-yloxy)-2,3-dihydro-1,3-dioxo-1H-indene (Example 7, 229 mg, 0.748 mmol) in DMF was cooled to −40° C. in a dry ice MeCN bath and stirred for 5 min. To this was added dropwise, triethylamine (300 μl, 2.13 mmol) and the resulting mixture was stirred for 10 min. To this mixture was then added ethyl isocyanate (CAS 109-90-0, 150 μl, 1.90 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration to provide the title compound as the dark solid free acid (230.1 mg, 68%). LCMS (ESI+): R_{f}=6.31, M+H=449.44; HRMS (ESI−): calculated for C₂₄H₁₉N₂O₇ m/z [M−H]⁻: 447.119775, observed 447.120605; ¹H NMR 300 MHz DMSO δ: 8.70-8.84 (m, 1H), 7.76-7.64 (m, 1H), 7.22-7.30 (m, 1H), 7.13-7.19 (m, 1H), 3.28-3.44 (m, 2H), 1.08-1.21 (m, 3H).

### Example 335—Synthesis of N-ethyl-5-[2-(ethylcarbamoyl)-1,3-dioxo-2,3-dihydro-1H-indene-5-carbonyl]-1,3-dioxo-2,3-dihydro-1H-indene-2-carboxamide

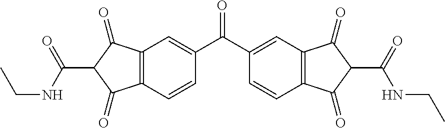 [SMILES: CCNC(=O)C1C(=O)c2ccc(C(=O)c3ccc4c(c3)C(=O)C(C(=O)NCC)C4=O)cc2C1=O]

A solution of 5,5′-carbonylbis(1H-indene-1,3(2H)-dione (Example 4, 240.0 mg, 0.755 mmol) in DMF was cooled to −40° C. in a dry ice MeCN bath and stirred for 5 min. To this was added dropwise, triethylamine (300 μl, 2.13 mmol) and the resulting mixture was stirred for 10 min. To this mixture was then added ethyl isocyanate (CAS 109-90-0, 150 μl, 1.90 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (296.1 mg, 78%). LCMS (ESI+): R_{f}=5.79, M+H=461.46; HRMS (ESI−): calculated for C₂₅H₁₉N₂O₇ m/z [M−H]⁻: 459.119775, observed 459.121041; ¹H NMR 300 MHz DMSO δ: 8.41-8.50 (m, 1H), 7.77-7.82 (m, 1H), 7.53-7.56 (m, 1H), 7.42-7.47 (m, 1H), 3.15-3.27 (m, 2H), 1.02-1.12 (m, 3H).

### Example 336—Synthesis of N-ethyl-5-{[2-(ethylcarbamoyl)-1,3-dioxo-2,3-dihydro-1H-inden-5-yl]sulfonyl}-1,3-dioxo-2,3-dihydro-1H-indene-2-carboxamide

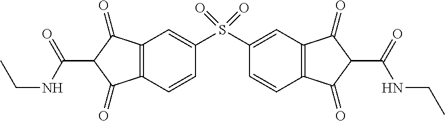 [SMILES: CCNC(=O)C1C(=O)c2ccc(S(=O)(=O)c3ccc4c(c3)C(=O)C(C(=O)NCC)C4=O)cc2C1=O]

A solution of 5,5′-sulfonylbis(1H-indene-1,3(2H)-dione) (Example 11, 276 mg, 0.779 mmol) in DMF was cooled to −40° C. in a dry ice MeCN bath and stirred for 5 min. To this was added dropwise, triethylamine (300 μl, 2.13 mmol) and the resulting mixture was stirred for 10 min. To this mixture was then added ethyl isocyanate (CAS 109-90-0, 150 μl, 1.89 mmol) dropwise to the reaction mixture. The reaction was then allowed to slowly warm to ambient temperature overnight. Once the reaction had been deemed complete by LCMS, the reaction was transferred to a water bath and quenched by the dropwise addition of 15 ml of 2N HCl, producing a thick slurry. The slurry was allowed to age for 5 min, then was isolated by filtration. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide the title compound as the dark solid bis-sodium salt (354.3 mg, 84%). LCMS (ESI+): R_{f}=5.78, M+H=497.44; HRMS (ESI−): calculated for C₂₄H₁₉N₂O₈S m/z [M−H]⁻: 495.086760, observed 495.086244; ¹H NMR 300 MHz DMSO δ: 8.32-8.40 (m, 1H), 8.01-8.074 (m, 1H), 7.61-7.65 (m, 1H), 7.45-7.51 (m, 1H), 3.13-3.23 (m, 2H), 0.99-1.10 (m, 3H).

### Example 337—Synthesis of tert-butyl N-[(2S)-1-[5-({2-[(2S)-2-{[(tert-butoxy)carbonyl]amino}-3-methylbutanoyl]-1,3-dioxo-2,3-dihydro-1H-inden-5-yl}oxy)-1,3-dioxo-2,3-dihydro-1H-inden-2-yl]-3-methyl-1-oxobutan-2-yl]carbamate

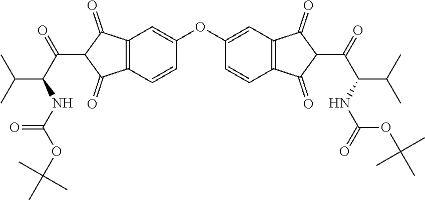 [SMILES: CC(C)[C@H](NC(=O)OC(C)(C)C)C(=O)C1C(=O)c2ccc(Oc3ccc4c(c3)C(=O)C(C(=O)[C@@H](NC(=O)OC(C)(C)C)C(C)C)C4=O)cc2C1=O]

To a solution of dimethylaminopyridine (DMAP, 183 mg, 1.50 mmol), Boc-L-Valine (CAS number 13734-41-4, 325.9 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 157.1 mg, 0.513 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (246.3 mg, 66%). LCMS (ESI+): R_{f}=7.06, M+Na=727.69; HRMS (ESI−): calculated for C₃₈H₄₃N₂O₁₁ m/z [M−H]⁻: 703.287234, observed 703.286899; ¹H NMR 300 MHz DMSO δ: 7.38-7.43 (m, 1H), 7.07-7.14 (m, 1H), 6.91-6.94 (m, 1H), 5.88-6.02 (m, 1H), 4.93-4.10 (m, 1H), 1.94-2.07 (m, 1H), 1.32-1.38 (m, 9H), 0.85-0.92 (m, 3H), 0.60-0.66 (m, 3H).

### Example 338—Synthesis of tert-butyl N-[(2S)-1-(5-{2-[(2S)-2-{[(tert-butoxy)carbonyl]amino}-3-methylbutanoyl]-1,3-dioxo-2,3-dihydro-1H-indene-5-carbonyl}-1,3-dioxo-2,3-dihydro-1H-inden-2-yl)-3-methyl-1-oxobutan-2-yl]carbamate

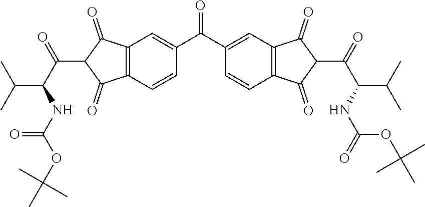 [SMILES: CC(C)[C@H](NC(=O)OC(C)(C)C)C(=O)C1C(=O)c2ccc(C(=O)c3ccc4c(c3)C(=O)C(C(=O)[C@@H](NC(=O)OC(C)(C)C)C(C)C)C4=O)cc2C1=O]

To a solution of dimethylaminopyridine (DMAP, 183 mg, 1.50 mmol), Boc-L-Valine (CAS number 13734-41-4, 325.9 mg, 1.50 mmol) in anhydrous DMF (10 ml) was added EDCI·HCl (286.5 mg, 1.50 mmol) and the contents were stirred for 10 min. 5,5′-carbonylbis(1H-indene-1,3(2H)-dione (Example 4, 159 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (303.8 mg, 80%). LCMS (ESI+): R_{f}=6.82, M+Na=739.71; HRMS (ESI−): calculated for C₃₉H₄₃N₂O₁₁ m/z [M−H]⁻: 715.287234, observed 715.285934; ¹H NMR 300 MHz DMSO δ: 7.83-7.89 (m, 1H), 7.62-7.66 (m, 1H), 7.51-7.57 (m, 1H), 5.92-6.00 (m, 1H), 5.00-5.09 (m, 1H), 1.97-2.07 (m, 1H), 1.31-1.40 (m, 9H), 0.87-0.95 (m, 3H), 0.61-0.67 (m, 3H).

### Example 339—Synthesis of tert-butyl N-[(2S)-1-[5-({2-[(2S)-2-{[(tert-butoxy)carbonyl]amino}-3-methylbutanoyl]-1,3-dioxo-2,3-dihydro-1H-inden-5-yl}sulfonyl)-1,3-dioxo-2,3-dihydro-1H-inden-2-yl]-3-methyl-1-oxobutan-2-yl]carbamate

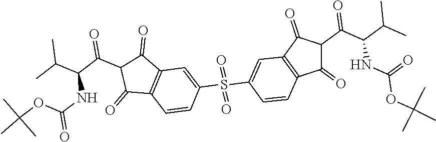 [SMILES: CC(C)[C@H](NC(=O)OC(C)(C)C)C(=O)C1C(=O)c2ccc(S(=O)(=O)c3ccc4c(c3)C(=O)C(C(=O)[C@@H](NC(=O)OC(C)(C)C)C(C)C)C4=O)cc2C1=O]

To a solution of dimethylaminopyridine (DMAP, 183 mg, 1.50 mmol), Boc-L-Valine (CAS number 13734-41-4, 325.9 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 11, 179 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (273.9 mg, 69%). LCMS (ESI+): R_{f}=6.82, M+Na=775.69; HRMS (ESI−): calculated for C₃₈H₄₃N₂O₁₁ m/z [M−H]⁻: 751.254219, observed 751.254053; ¹H NMR 300 MHz DMSO δ: 8.05-8.12 (m, 1H), 7.74-7.83 (m, 1H), 7.51-7.62 (m, 1H), 5.87-5.99 (m, 1H), 4.96-5.10 (m, 1H), 1.93-2.07 (m, 1H), 1.23-1.42 (m, 9H), 0.83-0.95 (m, 3H), 0.53-0.65 (m, 3H).

### Example 340—Synthesis of tert-butyl N-[(2R)-1-[5-({2-[(2R)-2-{[(tert-butoxy)carbonyl]amino}-3-methylbutanoyl]-1,3-dioxo-2,3-dihydro-1H-inden-5-yl}oxy)-1,3-dioxo-2,3-dihydro-1H-inden-2-yl]-3-methyl-1-oxobutan-2-yl]carbamate

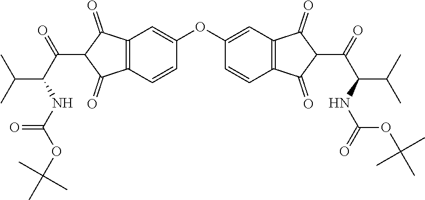 [SMILES: CC(C)[C@@H](NC(=O)OC(C)(C)C)C(=O)C1C(=O)c2ccc(Oc3ccc4c(c3)C(=O)C(C(=O)[C@H](NC(=O)OC(C)(C)C)C(C)C)C4=O)cc2C1=O]

To a solution of dimethylaminopyridine (DMAP, 183 mg, 1.50 mmol), Boc-D-Valine (CAS number 22838-58-0, 325.9 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 155 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (231.0 mg, 61%). LCMS (ESI+): R_{f}=6.84, M+Na=727.63; HRMS (ESI−): calculated for C₃₈H₄₃N₂O₁₁ m/z [M−H]⁻: 703.287234, observed 703.286904; ¹H NMR 300 MHz DMSO δ: 7.37-7.42 (m, 1H), 7.07-7.14 (m, 1H), 6.90-6.94 (m, 1H), 5.90-6.00 (m, 1H), 4.95-5.05 (m, 1H), 1.95-2.05 (m, 1H), 1.30-1.40 (m, 9H), 0.85-0.94 (m, 3H), 0.58-0.68 (m, 3H).

### Example 341—Synthesis of tert-butyl N-[(2R)-1-(5-{2-[(2R)-2-{[(tert-butoxy)carbonyl]amino}-3-methylbutanoyl]-1,3-dioxo-2,3-dihydro-1H-indene-5-carbonyl}-1,3-dioxo-2,3-dihydro-1H-inden-2-yl)-3-methyl-1-oxobutan-2-yl]carbamate

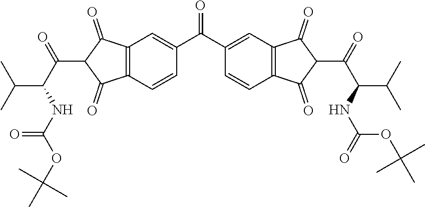 [SMILES: CC(C)[C@@H](NC(=O)OC(C)(C)C)C(=O)C1C(=O)c2ccc(C(=O)c3ccc4c(c3)C(=O)C(C(=O)[C@H](NC(=O)OC(C)(C)C)C(C)C)C4=O)cc2C1=O]

To a solution of dimethylaminopyridine (DMAP, 183 mg, 1.50 mmol), Boc-D-Valine (CAS number 22838-58-0, 325.9 mg, 1.50 mmol) in anhydrous DMF (10 ml) was added EDCI·HCl (286.5 mg, 1.50 mmol) and the contents were stirred for 10 min. 5,5′-carbonylbis(1H-indene-1,3(2H)-dione (Example 4, 159 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (277.7 mg, 73%). LCMS (ESI+): R_{f}=7.06, M+Na=739.57; HRMS (ESI−): calculated for C₃₉H₄₃N₂O₁₁ m/z [M−H]⁻: 715.287234, observed 715.286384; ¹H NMR 300 MHz DMSO δ: 7.83-7.88 (m, 1H), 7.62-7.66 (m, 1H), 7.51-7.57 (m, 1H), 5.92-6.00 (m, 1H), 2.00-2.07 (m, 1H), 1.31-1.40 (m, 9H), 0.87-0.94 (m, 3H), 0.60-0.68 (m, 3H).

### Example 342—Synthesis of tert-butyl N-[(2R)-1-[5-({2-[(2R)-2-{[(tert-butoxy)carbonyl]amino}-3-methylbutanoyl]-1,3-dioxo-2,3-dihydro-1H-inden-5-yl}sulfonyl)-1,3-dioxo-2,3-dihydro-1H-inden-2-yl]-3-methyl-1-oxobutan-2-yl]carbamate

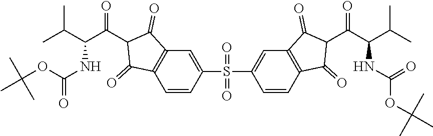 [SMILES: CC(C)[C@@H](NC(=O)OC(C)(C)C)C(=O)C1C(=O)c2ccc(S(=O)(=O)c3ccc4c(c3)C(=O)C(C(=O)[C@H](NC(=O)OC(C)(C)C)C(C)C)C4=O)cc2C1=O]

To a solution of dimethylaminopyridine (DMAP, 183 mg, 1.50 mmol), Boc-D-Valine (CAS number 22838-58-0, 325.9 mg, 1.50 mmol) in anhydrous DMF (10 ml) was added EDCI·HCl (286.5 mg, 1.50 mmol) and the contents were stirred for 10 min. 5,5′-sulfonylbis(1H-indene-1,3(2H)-dione) (Example 11, 179 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (254.1 mg, 63%). LCMS (ESI+): R_{f}=6.99, M+Na=775.55; HRMS (ESI−): calculated for C₃₈H₄₃N₂O₁₁ m/z [M−H]⁻: 751.254219, observed 751.255926; ¹H NMR 300 MHz DMSO δ: 8.06-8.11 (m, 1H), 7.75-7.78 (m, 1H), 7.53-7.58 (m, 1H), 5.88-5.96 (m, 1H), 4.95-5.07 (m, 1H), 1.92-2.04 (m, 1H), 1.29-1.39 (m, 9H), 0.84-0.91 (m, 3H), 0.56-0.65 (m, 3H).

### Example 343—Synthesis of tert-butyl N-[(2R)-1-[5-({2-[(2R)-2-{[(tert-butoxy)carbonyl]amino}-4-methylpentanoyl]-1,3-dioxo-2,3-dihydro-1H-inden-5-yl}oxy)-1,3-dioxo-2,3-dihydro-1H-inden-2-yl]-4-methyl-1-oxopentan-2-yl]carbamate

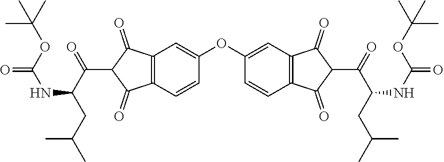 [SMILES: CC(C)C[C@@H](NC(=O)OC(C)(C)C)C(=O)C1C(=O)c2ccc(Oc3ccc4c(c3)C(=O)C(C(=O)[C@@H](CC(C)C)NC(=O)OC(C)(C)C)C4=O)cc2C1=O]

To a solution of dimethylaminopyridine (DMAP, 183 mg, 1.50 mmol), Boc-D-Leucine (CAS number 16937-99-8, 346.9 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 155 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (238.0 mg, 61%). LCMS (ESI+): R_{f}=7.36, M+Na=755.71; HRMS (ESI−): calculated for C₄₀H₄₇N₂O₁₁ m/z [M−H]⁻: 731.318534, observed 731.320116; ¹H NMR 300 MHz DMSO δ: 7.35-7.45 (m, 1H), 7.05-7.16 (m, 1H), 6.87-6.95 (m, 1H), 6.19-6.30 (m, 1H), 5.01-5.15 (m, 1H), 1.55-1.73 (m, 1H), 1.28-1.40 (m, 9H), 1.16-1.28 (m, 2H), 0.92-1.01 (m, 3H), 0.75-0.87 (m, 3H).

### Example 344—Synthesis of tert-butyl N-[(2R)-1-(5-{2-[(2R)-2-{[(tert-butoxy)carbonyl]amino}-4-methylpentanoyl]-1,3-dioxo-2,3-dihydro-1H-indene-5-carbonyl}-1,3-dioxo-2,3-dihydro-1H-inden-2-yl)-4-methyl-1-oxopentan-2-yl]carbamate

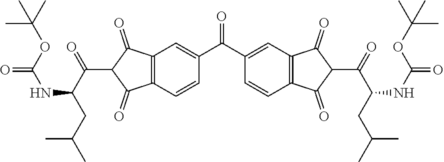 [SMILES: CC(C)C[C@@H](NC(=O)OC(C)(C)C)C(=O)C1C(=O)c2ccc(C(=O)c3ccc4c(c3)C(=O)C(C(=O)[C@@H](CC(C)C)NC(=O)OC(C)(C)C)C4=O)cc2C1=O]

To a solution of dimethylaminopyridine (DMAP, 183 mg, 1.50 mmol), Boc-D-Leucine (CAS number 16937-99-8, 346.9 mg, 1.50 mmol) in anhydrous DMF (10 ml) was added EDCI·HCl (286.5 mg, 1.50 mmol) and the contents were stirred for 10 min. 5,5′-carbonylbis(1H-indene-1,3(2H)-dione (Example 4, 159 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (310.1 mg, 79%). LCMS (ESI+): R_{f}=7.17, M+Na=767.66; HRMS (ESI−): calculated for C₄₁H₄₇N₂O₁₁ m/z [M−H]⁻: 743.318534, observed 743.320200; ¹H NMR 300 MHz DMSO δ: 7.81-7-95 (m, 1H), 7.62-7.70 (m, 1H), 6.19-6.35 (m, 1H), 5.04-5.24 (m, 1H), 1.58-1.76 (m, 1H), 1.18-1.45 (m, 11H), 0.91-1.02 (m, 3H), 0.76-0.87 (m, 3H).

### Example 345—Synthesis of tert-butyl N-[(2R)-1-[5-({2-[(2R)-2-{[(tert-butoxy)carbonyl]amino}-4-methylpentanoyl]-1,3-dioxo-2,3-dihydro-1H-inden-5-yl}sulfonyl)-1,3-dioxo-2,3-dihydro-1H-inden-2-yl]-4-methyl-1-oxopentan-2-yl]carbamate

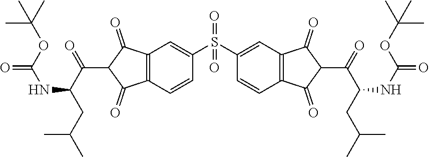 [SMILES: CC(C)C[C@@H](NC(=O)OC(C)(C)C)C(=O)C1C(=O)c2ccc(S(=O)(=O)c3ccc4c(c3)C(=O)C(C(=O)[C@@H](CC(C)C)NC(=O)OC(C)(C)C)C4=O)cc2C1=O]

To a solution of dimethylaminopyridine (DMAP, 183 mg, 1.50 mmol), Boc-D-Leucine (CAS number 16937-99-8, 346.9 mg, 1.50 mmol) in anhydrous DMF (10 ml) was added EDCI·HCl (286.5 mg, 1.50 mmol) and the contents were stirred for 10 min. 5,5′-sulfonylbis(1H-indene-1,3(2H)-dione) (Example 11, 179 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (313.3 mg, 76%). LCMS (ESI+): R_{f}=7.25, M+Na=803.64; HRMS (ESI−): calculated for C₄₀H₄₇N₂O₁₂S m/z [M−H]⁻: 779.285520, observed 779.287518; ¹H NMR 300 MHz DMSO δ: 8.03-8.14 (m, 1H), 7.75-7.80 (m, 1H), 7.51-7.62 (m, 1H), 6.18-6.27 (m, 1H), 5.00-5.18 (m, 1H), 1.55-1.71 (m, 1H), 1.13-1.42 (m, 11H), 0.89-1.01 (m, 3H), 0.75-0.84 (m, 3H).

### Example 346—Synthesis of tert-butyl N-[(2S)-1-[5-({2-[(2S)-2-{[(tert-butoxy)carbonyl]amino}-4-methylpentanoyl]-1,3-dioxo-2,3-dihydro-1H-inden-5-yl}oxy)-1,3-dioxo-2,3-dihydro-1H-inden-2-yl]-4-methyl-1-oxopentan-2-yl]carbamate

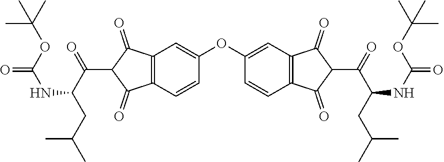 [SMILES: CC(C)C[C@H](NC(=O)OC(C)(C)C)C(=O)C1C(=O)c2ccc(Oc3ccc4c(c3)C(=O)C(C(=O)[C@H](CC(C)C)NC(=O)OC(C)(C)C)C4=O)cc2C1=O]

To a solution of dimethylaminopyridine (DMAP, 183 mg, 1.50 mmol), Boc-L-Leucine (CAS number 13139-15-6, 346.9 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 155 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (233.0 mg, 60%). LCMS (ESI+): R_{f}=7.32, M+Na=755.71; HRMS (ESI−): calculated for C₄₀H₄₇N₂O₁₁ m/z [M−H]⁻: 731.318534, observed 731.318290; ¹H NMR 300 MHz DMSO δ: 7.38-7.45 (m, 1H), 7.07-7.14 (m, 1H), 6.89-6.95 (m, 1H), 6.19-6.28 (m, 1H), 5.04-5.16 (m, 1H), 1.55-1.76 (m, 1H), 1.19-1.40 (m, 11H), 0.93-1.01 (m, 3H), 0.78-0.86 (M, 3H).

### Example 347—Synthesis of tert-butyl N-[(2S)-1-(5-{2-[(2S)-2-{[(tert-butoxy)carbonyl]amino}-4-methylpentanoyl]-1,3-dioxo-2,3-dihydro-1H-indene-5-carbonyl}-1,3-dioxo-2,3-dihydro-1H-inden-2-yl)-4-methyl-1-oxopentan-2-yl]carbamate

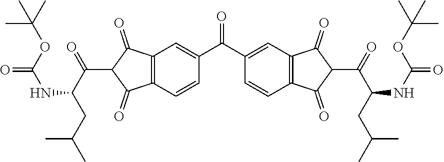 [SMILES: CC(C)C[C@H](NC(=O)OC(C)(C)C)C(=O)C1C(=O)c2ccc(C(=O)c3ccc4c(c3)C(=O)C(C(=O)[C@H](CC(C)C)NC(=O)OC(C)(C)C)C4=O)cc2C1=O]

To a solution of dimethylaminopyridine (DMAP, 183 mg, 1.50 mmol), Boc-L-Leucine (CAS number 13139-15-6, 346.9 mg, 1.50 mmol) in anhydrous DMF (10 ml) was added EDCI·HCl (286.5 mg, 1.50 mmol) and the contents were stirred for 10 min. 5,5′-carbonylbis(1H-indene-1,3(2H)-dione (Example 4, 159 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (307.6 mg, 78%). LCMS (ESI+): R_{f}=7.17, M+Na=767.66; HRMS (ESI−): calculated for C₄₁H₄₇N₂O₁₁ m/z [M−H]⁻: 743.318534, observed 743.318272; ¹H NMR 300 MHz DMSO δ: 7.83-7.92 (m, 1H), 7.62-7.69 (m, 1H), 7.52-7.60 (m, 1H), 6.23-6.36 (m, 1H), 5.06-5.21 (m, 1H), 1.60-1.75 (m, 1H), 1.20-1.45 (m, 11H), 0.94-1.05 (m, 3H), 0.78-0.87 (m, 3H).

### Example 348—Synthesis of tert-butyl N-[(2S)-1-[5-({2-[(2S)-2-{[(tert-butoxy)carbonyl]amino}-4-methylpentanoyl]-1,3-dioxo-2,3-dihydro-1H-inden-5-yl}sulfonyl)-1,3-dioxo-2,3-dihydro-1H-inden-2-yl]-4-methyl-1-oxopentan-2-yl]carbamate

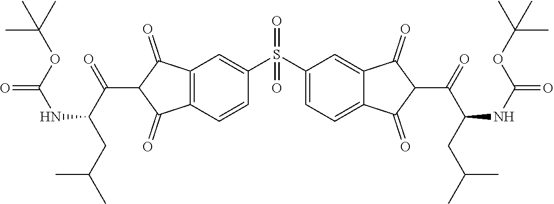 [SMILES: CC(C)C[C@H](NC(=O)OC(C)(C)C)C(=O)C1C(=O)c2ccc(S(=O)(=O)c3ccc4c(c3)C(=O)C(C(=O)[C@H](CC(C)C)NC(=O)OC(C)(C)C)C4=O)cc2C1=O]

To a solution of dimethylaminopyridine (DMAP, 183 mg, 1.50 mmol), Boc-L-Leucine (CAS number 13139-15-6, 346.9 mg, 1.50 mmol) in anhydrous DMF (10 ml) was added EDCI·HCl (286.5 mg, 1.50 mmol) and the contents were stirred for 10 min. 5,5′-Sulfonylbis(1H-indene-1,3(2H)-dione) (Example 11, 179 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder to provide the title compound as the free acid (305.2 mg, 74%). LCMS (ESI+): R_{f}=7.13, M+Na=803.64; HRMS (ESI−): calculated for C₄₀H₄₇N₂O₁₂S m/z [M−H]⁻: 779.285520, observed 779.284614; ¹H NMR 300 MHz DMSO δ: 8.07-8.14 (m, 1H), 7.77-7.80 (m, 1H), 7.54-7.61 (m, 1H), 6.19-6.29 (m, 1H), 5.02-5.15 (m, 1H), 1.57-1.70 (m, 1H), 1.10-1.39 (m, 11H), 0.92-0.99 (m, 3H), 0.75-0.83 (m, 3H).

### Example 349—Synthesis of 5-{1,3-dioxo-2-[(3R)-pyrrolidine-3-carbonyl]-2,3-dihydro-1H-indene-5-carbonyl}-2-[(2R)-pyrrolidine-2-carbonyl]-2,3-dihydro-1H-indene-1,3-dione

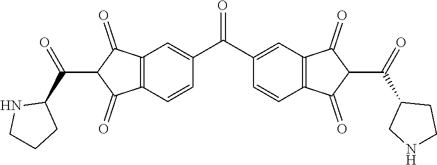 [SMILES: O=C(c1ccc2c(c1)C(=O)C(C(=O)[C@@H]1CCNC1)C2=O)c1ccc2c(c1)C(=O)C(C(=O)[C@H]1CCCN1)C2=O]

To a slurry of tert-butyl (2R)-2-(5-{2-[(3R)-1-[(tert-butoxy)carbonyl]pyrrolidine-3-carbonyl]-1,3-dioxo-2,3-dihydro-1H-indene-5-carbonyl}-1,3-dioxo-2,3-dihydro-1H-indene-2-carbonyl)pyrrolidine-1-carboxylate bis sodium salt (Example 161, 81.4 mg, 0.108 mmol) in 2.5 ml of DCM was charged 1 ml of trifluoroacetic acid (TFA, 1.0 ml, 13.06 mmol). The addition of the TFA produced a dark brown solution. The reaction was stirred at ambient temperature. Once the reaction was deemed complete by LCMS, 2.5 ml of H₂O was added and the resulting precipitant was filtered. The solid was washed with 5 ml of DCM. The tan solid was dried overnight in the vacuum oven. The title compound was obtained as the bis trifluoroacetic acid salt (48.1 mg, 63% yield). LCMS (ESI+): R_{f}=2.96, M+H=513.51; HRMS (ESI−): calculated for C₂₉H₂₃N₂O₇ m/z [M−H]⁻: Exact Mass: 511.151075, observed 511.151740; ¹H NMR 300 MHz DMSO δ: 8.71-8.83 (m, 1H), 8.27-8.41 (m, 1H), 7.86-7.92 (m, 1H), 7.66-7.70 (m, 1H), 7.57-7.63 (m, 1H), 4.94-5.06 (m, 1H), 3.13-3.35 (m, 4H), 1.65-1.95 (m, 4H).

### Example 350—Synthesis of 5-({1,3-dioxo-2-[(3R)-pyrrolidine-3-carbonyl]-2,3-dihydro-1H-inden-5-yl}sulfonyl)-2-[(2R)-pyrrolidine-2-carbonyl]-2,3-dihydro-1H-indene-1,3-dione

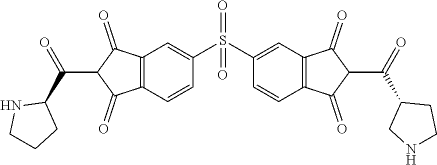 [SMILES: O=C1c2ccc(S(=O)(=O)c3ccc4c(c3)C(=O)C(C(=O)[C@H]3CCCN3)C4=O)cc2C(=O)C1C(=O)[C@@H]1CCNC1]

To a slurry of tert-butyl (2R)-2-[5-({2-[(2R)-1-[(tert-butoxy)carbonyl]pyrrolidine-2-carbonyl]-1,3-dioxo-2,3-dihydro-1H-inden-5-yl}sulfonyl)-1,3-dioxo-2,3-dihydro-1H-indene-2-carbonyl]pyrrolidine-1-carboxylate, bis sodium salt (Example 162, 88.2 mg, 0.104 mmol) in 2.5 ml of DCM was charged 1 ml of trifluoroacetic acid (TFA, 1.0 ml, 13.06 mmol). The addition of the TFA produced a dark brown solution. The reaction was stirred at ambient temperature. Once the reaction was deemed complete by LCMS, 2.5 ml of H₂O was added and the resulting precipitant was filtered. The solid was washed with 5 ml of DCM. The tan solid was dried overnight in the vacuum oven. The title compound was obtained as the bis trifluoroacetic acid salt (51.7 mg, 67% yield). LCMS (ESI+): R_{f}=3.04, M+H=549.42; HRMS (ESI−): calculated for C₂₈H₂₃N₂O₈S m/z [M−H]⁻: Exact Mass: 547.118060, observed 547.118739; ¹H NMR 300 MHz DMSO δ: 8.70-8.82 (m, 1H), 8.27-8.37 (m, 1H), 8.13-8.18 (m, 1H), 7.81-7.85 (m, 1H), 7.60-7.65 (m, 1H), 3.11-3.32 (m, 4H), 1.60-1.93 (m, 4H).

### Example 351—Synthesis of 2-[(2R)-2-amino-3-methylbutanoyl]-5-({2-[(2R)-2-amino-3-methylbutanoyl]-1,3-dioxo-2,3-dihydro-1H-inden-5-yl}oxy)-2,3-dihydro-1H-indene-1,3-dione

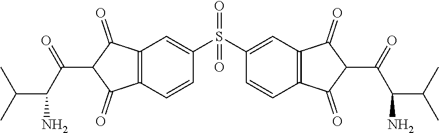 [SMILES: CC(C)[C@@H](N)C(=O)C1C(=O)c2ccc(S(=O)(=O)c3ccc4c(c3)C(=O)C(C(=O)[C@H](N)C(C)C)C4=O)cc2C1=O]

To a slurry of tert-butyl N-[(2R)-1-[5-({2-[(2R)-2-{[(tert-butoxy)carbonyl]amino}-3-methylbutanoyl]-1,3-dioxo-2,3-dihydro-1H-inden-5-yl}oxy)-1,3-dioxo-2,3-dihydro-1H-inden-2-yl]-3-methyl-1-oxobutan-2-yl]carbamate bis sodium salt (Example 340, 86.6 mg, 0.116 mmol) in 5 ml of DCM was charged 1 ml of trifluoroacetic acid (TFA, 1 ml, 13.06 mmol). The addition of the TFA produced a dark brown solution. The reaction was stirred at ambient temperature. Once the reaction was deemed complete by LCMS, 2.5 ml of H₂O was added and the resulting precipitant was filtered. The solid was washed with 5 ml of DCM. The tan solid was dried overnight in the vacuum oven. The title compound was obtained as the bis trifluoroacetic acid salt (65.9 mg, 81% yield). LCMS (ESI+): R_{f}=3.33, M+H=505.54; HRMS (ESI−): calculated for C₂₈H₂₇N₂O₇ m/z [M−H]⁻: Exact Mass: 503.182375, observed 503.182652; ¹H NMR 300 MHz DMSO δ: 7.59-7.71 (m, 3H), 7.42-7.47 (m, 1H), 7.12-7.18 (m, 1H), 6.92-6.95 (m, 1H), 4.70-4.80 (m, 1H), 0.96-1.05 (m, 3H), 0.74-0.83 (m, 3H).

### Example 352—Synthesis of 2-[(2R)-2-amino-3-methylbutanoyl]-5-{2-[(2R)-2-amino-3-methylbutanoyl]-1,3-dioxo-2,3-dihydro-1H-indene-5-carbonyl}-2,3-dihydro-1H-indene-1,3-dione

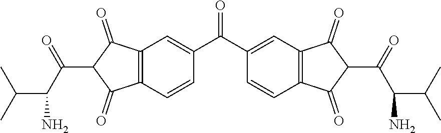 [SMILES: CC(C)[C@@H](N)C(=O)C1C(=O)c2ccc(C(=O)c3ccc4c(c3)C(=O)C(C(=O)[C@H](N)C(C)C)C4=O)cc2C1=O]

To a slurry of tert-butyl N-[(2R)-1-(5-{2-[(2R)-2-{[(tert-butoxy)carbonyl]amino}-3-methylbutanoyl]-1,3-dioxo-2,3-dihydro-1H-indene-5-carbonyl}-1,3-dioxo-2,3-dihydro-1H-inden-2-yl)-3-methyl-1-oxobutan-2-yl]carbamate, sodium salt (Example 341, 89.6 mg, 0.118 mmol) in 2.5 ml of DCM was charged 1 ml of trifluoroacetic acid (TFA, 1 ml, 13.06 mmol). The addition of the TFA produced a dark brown solution. The reaction was stirred at ambient temperature. Once the reaction was deemed complete by LCMS, 2.5 ml of H₂O was added and the resulting precipitant was filtered. The solid was washed with 5 ml of DCM. The tan solid was dried overnight in the vacuum oven. The title compound was obtained as the bis trifluoroacetic acid salt (49 mg, 58% yield). LCMS (ESI+): R_{f}=3.25, M+H=517.56; HRMS (ESI−): calculated for C₂₉H₂₇N₂O₇ m/z [M−H]⁻: Exact Mass: 515.182375, observed 515.182858; ¹H NMR 300 MHz DMSO δ: 7.86-7.91 (m, 1H), 7.65-7.76 (m, 4H), 7.56-7.61 (m, 1H), 4.74-4.83 (m, 1H), 2.08-2.21 (m, 1H), 0.99-1.06 (m, 3H), 0.75-0.85 (m, 3H).

### Example 353—Synthesis of 2-[(2R)-2-amino-3-methylbutanoyl]-5-({2-[(2R)-2-amino-3-methylbutanoyl]-1,3-dioxo-2,3-dihydro-1H-inden-5-yl}sulfonyl)-2,3-dihydro-1H-indene-1,3-dione

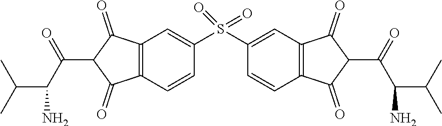 [SMILES: CC(C)[C@@H](N)C(=O)C1C(=O)c2ccc(S(=O)(=O)c3ccc4c(c3)C(=O)C(C(=O)[C@H](N)C(C)C)C4=O)cc2C1=O]

To a slurry of tert-butyl N-[(2R)-1-[5-({2-[(2R)-2-{[(tert-butoxy)carbonyl]amino}-3-methylbutanoyl]-1,3-dioxo-2,3-dihydro-1H-inden-5-yl}sulfonyl)-1,3-dioxo-2,3-dihydro-1H-inden-2-yl]-3-methyl-1-oxobutan-2-yl]carbamate, bis sodium salt (Example 342, 95.1 mg, 0.120 mmol) in 2.5 ml of DCM was charged 1 ml of TFA (13.06 mmol). The addition of the TFA produced a dark brown solution. The reaction was stirred at ambient temperature. Once the reaction was deemed complete by LCMS, 2.5 ml of H₂O was added and the resulting precipitant was filtered. The solid was washed with 5 ml of DCM. The tan solid was dried overnight in the vacuum oven. The title compound was obtained as the bis trifluoroacetic acid salt (64.5 mg, 72% yield). LCMS (ESI+): R_{f}=3.28, M+H=553.54; HRMS (ESI−): calculated for C₂₈H₂₇N₂O₈S m/z [M−H]⁻: Exact Mass: 551.149360, observed 551.150949; ¹H NMR 300 MHz DMSO δ: 8.12-8.17 (m, 1H), 7.81-7.84 (m, 1H), 7.66-7.76 (m, 1H), 7.59-7.65 (m, 1H), 4.69-4.79 (m, 1H), 2.05-2.19 (m, 1H), 0.95-1.03 (m, 3H), 0.72-0.81 (m, 3H).

### Example 354—Synthesis of 2-[(2R)-2-amino-4-methylpentanoyl]-5-{2-[(2R)-2-amino-4-methylpentanoyl]-1,3-dioxo-2,3-dihydro-1H-indene-5-carbonyl}-2,3-dihydro-1H-indene-1,3-dione

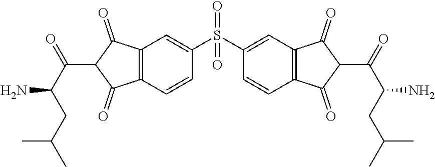 [SMILES: CC(C)C[C@@H](N)C(=O)C1C(=O)c2ccc(S(=O)(=O)c3ccc4c(c3)C(=O)C(C(=O)[C@H](N)CC(C)C)C4=O)cc2C1=O]

To a slurry of tert-butyl N-[(2R)-1-(5-{2-[(2R)-2-{[(tert-butoxy)carbonyl]amino}-4-methylpentanoyl]-1,3-dioxo-2,3-dihydro-1H-indene-5-carbonyl}-1,3-dioxo-2,3-dihydro-1H-inden-2-yl)-4-methyl-1-oxopentan-2-yl]carbamate, bis sodium salt (Example 343, 105.6 mg, 0.134 mmol) in 2.5 ml of DCM was charged 1 ml of TFA (13.06 mmol). The addition of the TFA produced a dark brown solution. The reaction was stirred at ambient temperature. Once the reaction was deemed complete by LCMS, 2.5 ml of H₂O was added and the resulting precipitant was filtered. The solid was washed with 5 ml of DCM. The tan solid was dried overnight in the vacuum oven. The title compound was obtained as the bis trifluoroacetic acid salt (51.7 mg, 52% yield). LCMS (ESI+): R_{f}=3.54, M+H=545.57; HRMS (ESI−): calculated for C₃₁H₃₁N₂O₇ m/z [M−H]⁻: Exact Mass: 543.213675, observed 543.212748; ¹H NMR 300 MHz DMSO δ: 7.85-7.91 (m, 1H), 7.72-7.85 (m, 3H), 7.65-7.71 (m, 1H), 4.85-4.99 (m, 1H), 1.63-1.79 (m, 1H), 1.37-1.60 (m, 2H), 0.95-1.04 (m, 3H), 0.80-0.92 (m, 3H).

### Example 355—Synthesis of 2-[(2R)-2-amino-4-methylpentanoyl]-5-({2-[(2R)-2-amino-4-methylpentanoyl]-1,3-dioxo-2,3-dihydro-1H-inden-5-yl}sulfonyl)-2,3-dihydro-1H-indene-1,3-dione

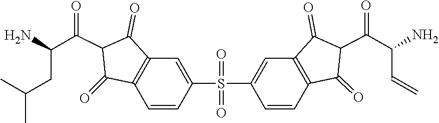 [SMILES: C=C[C@@H](N)C(=O)C1C(=O)c2ccc(S(=O)(=O)c3ccc4c(c3)C(=O)C(C(=O)[C@H](N)CC(C)C)C4=O)cc2C1=O]

To a slurry of tert-butyl N-[(2R)-1-[5-({2-[(2R)-2-{[(tert-butoxy)carbonyl]amino}-4-methylpentanoyl]-1,3-dioxo-2,3-dihydro-1H-inden-5-yl}sulfonyl)-1,3-dioxo-2,3-dihydro-1H-inden-2-yl]-4-methyl-1-oxopentan-2-yl]carbamate, bis sodium salt (Example 344, 98.8 mg, 0.120 mmol) in 2.5 ml of DCM was charged 1 ml TFA (13.06 mmol). The addition of the TFA produced a dark brown solution. The reaction was stirred at ambient temperature. Once the reaction was deemed complete by LCMS, 2.5 ml of H₂O was added and the resulting precipitant was filtered. The solid was washed with 5 ml of DCM. The tan solid was dried overnight in the vacuum oven. The title compound was obtained as the bis trifluoroacetic acid salt (45.3 mg, 49% yield). LCMS (ESI+): R_{f}=3.54, M+H=581.55; HRMS (ESI−): calculated for C₃₀H₃₁N₂O₈S m/z [M−H]⁻: Exact Mass: 579.180661, observed 579.181212; ¹H NMR 300 MHz DMSO δ: 8.13-8.18 (m, 1H), 7.82-7.87 (m, 1H), 7.82-7.87 (m, 1H), 7.73-7.87 (m, 2H), 7.59-7.65 (m, 1H), 4.79-4.91 (m, 1H), 1.61-1.76 (m, 1H), 1.33-1.55 (m, 2H), 0.92-1.01 (m, 3H), 0.76-0.87 (m, 3H).

### Example 356—Synthesis of 5-({1,3-dioxo-2-[(3R)-pyrrolidine-3-carbonyl]-2,3-dihydro-1H-inden-5-yl}oxy)-2-[(2R)-pyrrolidine-2-carbonyl]-2,3-dihydro-1H-indene-1,3-dione

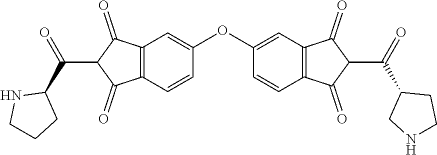 [SMILES: O=C1c2ccc(Oc3ccc4c(c3)C(=O)C(C(=O)[C@H]3CCCN3)C4=O)cc2C(=O)C1C(=O)[C@@H]1CCNC1]

Step 1: To a solution of dimethylaminopyridine (DMAP, 183 mg, 1.50 mmol), Boc-D-Proline (CAS number 37784-17-1, 322.9 mg, 1.50 mmol) in anhydrous DMF (10 ml) was added EDCI·HCl (286.5 mg, 1.50 mmol) and the contents were stirred for 10 min. 5-(2,3-dihydro-1,3-dioxo-1H-inden-6-yloxy)-2,3-dihydro-1,3-dioxo-1H-indene (Example 7, 155 mg, 0.500 mmol) was added and the reaction solution was stirred at ambient temperature. Once the reaction was deemed complete by LCMS, 15 ml of 2N HCl was added and the resulting precipitant was filtered. The solid was washed once with 5 ml of 2N HCl and 5 ml of water. The dried solid was milled into a fine powder and was treated with 10 ml of 1M NaOH and 5 ml of methylene chloride. This biphasic suspension was shaken vigorously and filtered to provide tert-butyl (2R)-2-[5-({2-[(3R)-1-[(tert-butoxy)carbonyl]pyrrolidine-3-carbonyl]-1,3-dioxo-2,3-dihydro-1H-inden-5-yl}oxy)-1,3-dioxo-2,3-dihydro-1H-indene-2-carbonyl]pyrrolidine-1-carboxylate as a dark solid bis-sodium salt (184.3 mg, 49%). LCMS (ESI+): R_{f}=7.32, M+Na=723.58; HRMS (ESI−): calculated for C₃₈H₃₉N₂O₁₁ m/z [M−H]⁻: 699.255934, observed 699.255738; ¹H NMR 300 MHz DMSO δ: 7.33-7.41 (m, 1H), 7.0-7.12 (m, 1H), 6.85-6.92 (m, 1H), 5.23-5.42 (m, 1H), 1.60-1.75 (m, 2H), 1.33-1.38 (m, 4H), 1.15-1.23 (m, 9H).

Step 2: To a slurry of tert-butyl (2R)-2-[5-({2-[(3R)-1-[(tert-butoxy) carbonyl]pyrrolidine-3-carbonyl]-1,3-dioxo-2,3-dihydro-1H-inden-5-yl}oxy)-1,3-dioxo-2,3-dihydro-1H-indene-2-carbonyl]pyrrolidine-1-carboxylate bis sodium salt (75.1 mg, 0.101 mmol) in 2.5 ml of DCM was charged 1 ml of TFA (13.06 mmol). The addition of the TFA produced a dark brown solution. The reaction was stirred at ambient temperature. Once the reaction was deemed complete by LCMS, 2.5 ml of H₂O was added and the resulting precipitant was filtered. The solid was washed with 5 ml of DCM. The tan solid was dried overnight in the vacuum oven. The title compound was obtained as the bis trifluoroacetic acid salt (50.3 mg, 72% yield). LCMS (ESI+): R_{f}=3.03, M+H=501.56; HRMS (ESI−): calculated for C₂₈H₂₃N₂O₇ m/z [M−H]⁻: Exact Mass: 499.151075, observed 499.150727; ¹H NMR 300 MHz DMSO δ: 8.68-8.80 (m, 1H), 8.25-8.41 (m, 1H), 7.44-7.49 (m, 1H), 7.14-7.20 (m, 2H), 6.93-6.98 (m, 1H), 4.91-5.04 (m, 1H), 3.09-3.34 (m, 4H), 1.62-1.94 (m, 4H).

### Example 357—Synthesis of 2-[(2R)-2-amino-4-methylpentanoyl]-5-({2-[(2R)-2-amino-4-methylpentanoyl]-1,3-dioxo-2,3-dihydro-1H-inden-5-yl}oxy)-2,3-dihydro-1H-indene-1,3-dione. FC-12227

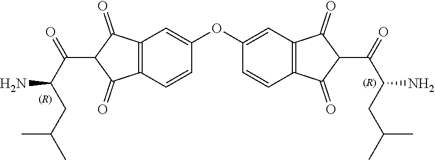

To a slurry of tert-butyl N-[(2R)-1-[5-({2-[(2R)-2-{[(tert-butoxy)carbonyl]amino}-4-methylpentanoyl]-1,3-dioxo-2,3-dihydro-1H-inden-5-yl}oxy)-1,3-dioxo-2,3-dihydro-1H-inden-2-yl]-4-methyl-1-oxopentan-2-yl]carbamate bis sodium salt (Example 343, 105.6 mg, 0.134 mmol) in 2.5 ml of DCM was charged 1 ml of TFA (13.06 mmol). The addition of the TFA produced a dark brown solution. The reaction was stirred at ambient temperature. Once the reaction was deemed complete by LCMS, 2.5 ml of H₂O was added and the resulting precipitant was filtered. The solid was washed with 5 ml of DCM. The tan solid was dried overnight in the vacuum oven. The title compound was obtained as the bis trifluoroacetic acid salt (48.8 mg, 53% yield). LCMS (ESI+): R_{f}=3.58, M+H=533.49; ¹H NMR δ: 7.67-7.83 (m, 4H), 7.43-7.48 (m, 1H), 7.11-7.18 (m, 1H), 4.80-4.94 (m, 1H), 1.61-1.74 (m, 1H), 1.33-1.57 (m, 2H), 0.91-1.00 (m, 3H), 0.75-0.89 (m, 3H).

### Example 358—Synthesis of N-[(1,1-dioxo-1λ⁶-thiolan-3-yl)methyl]-5-[(2-{[(1,1-dioxo-1λ⁶-thiolan-3-yl)methyl]carbamoyl}-1,3-dioxo-2,3-dihydro-1H-inden-5-yl)sulfonyl]-1,3-dioxo-2,3-dihydro-1H-indene-2-carboxamide. FC-12362

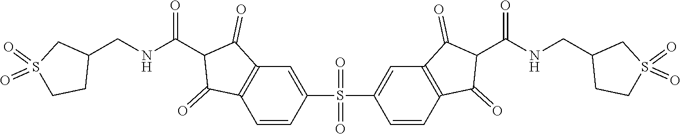 [SMILES: O=C(NCC1CCS(=O)(=O)C1)C1C(=O)c2ccc(S(=O)(=O)c3ccc4c(c3)C(=O)C(C(=O)NCC3CCS(=O)(=O)C3)C4=O)cc2C1=O]

Using the procedure of Example 336, reaction of 5,5′-sulfonylbis(1H-indene-1,3(2H)-dione) (Example 11) and isocyanatomethyl)tetrahydrothiophene-1,1-dioxide (CAS 2880-41-1) provided the title compound as a brown solid. The compound was isolated as the bis sodium salt. LCMS (ESI+): R_{f}=4.13, M+H=705.49; HRMS (ESI−): calculated for C₃₀H₂₇N₂O₁₂S₃ m/z [M−H]⁻: 703.0732, observed 703.0723; ¹H NMR δ: 8.52-8.60 (m, 1H), 8.01-8.08 (m, 1H), 7.62-7.66 (m, 1H), 7.45-7.52 (m, 1H), 2.94-3.24 (m, 4H), 2.69-2.82 (m, 1H), 2.07-2.22 (m, 1H), 1.69-1.86 (m, 1H).

### Example 359—Synthesis of N-[(1,1-dioxo-1λ⁶-thiolan-3-yl)methyl]-5-(2-{[(1,1-dioxo-1λ⁶-thiolan-3-yl)methyl]carbamoyl}-1,3-dioxo-2,3-dihydro-1H-indene-5-carbonyl)-1,3-dioxo-2,3-dihydro-1H-indene-2-carboxamide. FC-12363

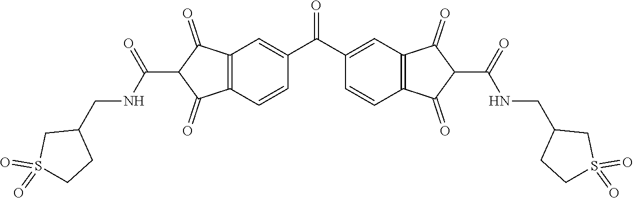 [SMILES: O=C(c1ccc2c(c1)C(=O)C(C(=O)NCC1CCS(=O)(=O)C1)C2=O)c1ccc2c(c1)C(=O)C(C(=O)NCC1CCS(=O)(=O)C1)C2=O]

Using the procedure of Example 335, reaction of 5,5′-carbonylbis(1H-indene-1,3(2H)-dione (Example 4) and 3-(isocyanatomethyl)tetrahydrothiophene-1,1-dioxide, (CAS 2880-41-) provided the title compound as a brown solid. The compound was isolated as the bis sodium salt. LCMS (ESI+): R_{f}=4.39, M+H=669.51; HRMS (ESI−): calculated for C₃₁H₂₇N₂O₁₁S₂ m/z [M−H]⁻: 667.1062, observed 667.1082; ¹H NMR δ: 8.61-8.70 (m, 1H), 7.77-7.83 (m, 1H), 7.52-7.57 (m, 1H), 7.41-7.49 (m, 1H), 2.96-3.22 (m, 4H), 2.69-2.88 (m, 1H), 2.52-2.65 (m, 1H), 2.10-2.27 (m, 1H), 1.71-1.89 (m, 1H).

### Example 360—Synthesis of N-(1,1-dioxo-1λ⁶-thiolan-3-yl)-5-({2-[(1,1-dioxo-1λ⁶-thiolan-3-yl)carbamoyl]-1,3-dioxo-2,3-dihydro-1H-inden-5-yl}sulfonyl)-1,3-dioxo-2,3-dihydro-1H-indene-2-carboxamide. FC-12364

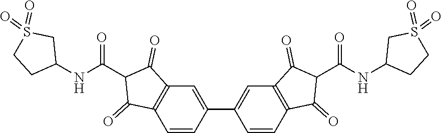 [SMILES: O=C(NC1CCS(=O)(=O)C1)C1C(=O)c2ccc(-c3ccc4c(c3)C(=O)C(C(=O)NC3CCS(=O)(=O)C3)C4=O)cc2C1=O]

Using the procedure of Example 336, reaction of 5,5′-sulfonylbis(1H-indene-1,3(2H)-dione) (Example 11) and 3-isocyanato-tetrahydro-thiophene-1,1-dioxide (CAS 24373-66-8) provided the title compound as a brown solid. The compound was isolated as the bis sodium salt. LCMS (ESI+): R_{f}=3.93, M+H=677.34; HRMS (ESI−): calculated for C₂₈H₂₃N₂O₁₂S₃ m/z [M−H]⁻: 675.0419, observed 675.0432; ¹H NMR δ: 8.66-8.73 (m, 1H), 7.62-7.67 (m, 1H), 7.44-7.52 (m, 1H), 7.44-7.52 (m, 1H), 4.48-4.62 (m, 1H), 3.34-3.43 (m, 1H), 3.06-3.26 (m, 4H), 2.90-3.01 (m, 1H), 2.31-2.44 (m, 2H), 2.00-2.15 (m, 1H).

### Example 361—Synthesis of N-(1,1-dioxo-1λ⁶-thiolan-3-yl)-5-{2-[(1,1-dioxo-1λ⁶-thiolan-3-yl)carbamoyl]-1,3-dioxo-2,3-dihydro-1H-indene-5-carbonyl}-1,3-dioxo-2,3-dihydro-1H-indene-2-carboxamide. FC-12365

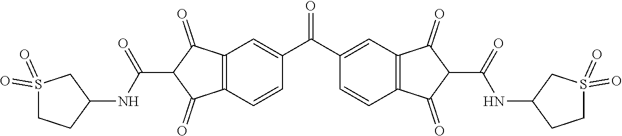 [SMILES: O=C(c1ccc2c(c1)C(=O)C(C(=O)NC1CCS(=O)(=O)C1)C2=O)c1ccc2c(c1)C(=O)C(C(=O)NC1CCS(=O)(=O)C1)C2=O]

Using the procedure of Example 335, reaction of 5,5′-carbonylbis(1H-indene-1,3(2H)-dione (Example 4) and 3-isocyanato-tetrahydro-thiophene-1,1-dioxide (CAS 24373-66-8) provided the title compound as a brown solid. The compound was isolated as the bis sodium salt. LCMS (ESI+): R_{f}=4.15, M+H=641.42; HRMS (ESI−): calculated for C₂₉H₂₃N₂O₁₁S₂ m/z [M−H]⁻: 639.0749, observed 639.0756; ¹H NMR δ: 8.75-8.84 (m, 1H), 7.77-7.84 (m, 1H), 7.53-7.58 (m, 1H), 7.42-7.49 (m, 1H), 4.50-4.67 (m, 1H), 3.35-3.45 (m, 2H), 2.93-3.04 (m, 1H), 2.34-2.43 (m, 1H), 2.04-2.20 (m, 1H).

### Example 362—Synthesis of 5-[(1,3-dioxo-2-{[(oxolan-3-yl)methyl]carbamoyl}-2,3-dihydro-1H-inden-5-yl)sulfonyl]-1,3-dioxo-N-[(oxolan-3-yl)methyl]-2,3-dihydro-1H-indene-2-carboxamide. FC-12376

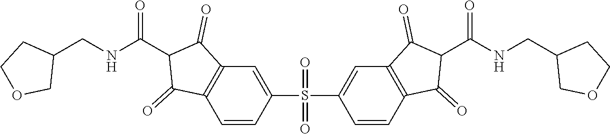 [SMILES: O=C(NCC1CCOC1)C1C(=O)c2ccc(S(=O)(=O)c3ccc4c(c3)C(=O)C(C(=O)NCC3CCOC3)C4=O)cc2C1=O]

Using the procedure of Example 336, reaction of 5,5′-sulfonylbis(1H-indene-1,3(2H)-dione) (Example 11) and 3-(isocyanatomethyl)oxolane (CAS 1341487-61-3) provided the title compound as a brown solid. The compound was isolated as the bis sodium salt. LCMS (ESI+): R_{f}=4.88, M+H=609.56; HRMS (ESI−): calculated for C₃₀H₂₇N₂O₁₀S m/z [M−H]⁻: 607.1392, observed 607.1406; ¹H NMR δ: 8.49-8.56 (m, 1H), 8.00-8.06 (m, 1H), 7.61-7.65 (m, 1H), 7.44-7.51 (m, 1H), 3.52-3.75 (m, 4H), 3.33-3.41 (m, 2H), 3.13-3.21 (m, 1H), 2.28-2.40 (m, 1H), 1.82-1.97 (m, 1H), 1.46-1.60 (m, 1H).

### Example 363—Synthesis of 5-(1,3-dioxo-2-{[(oxolan-3-yl)methyl]carbamoyl}-2,3-dihydro-1H-indene-5-carbonyl)-1,3-dioxo-N-[(oxolan-3-yl)methyl]-2,3-dihydro-1H-indene-2-carboxamide. FC-12377

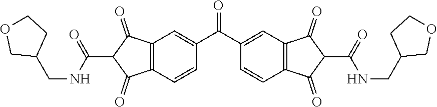 [SMILES: O=C(c1ccc2c(c1)C(=O)C(C(=O)NCC1CCOC1)C2=O)c1ccc2c(c1)C(=O)C(C(=O)NCC1CCOC1)C2=O]

Using the procedure of Example 335, reaction of 5,5′-carbonylbis(1H-indene-1,3(2H)-dione (Example 4) and 3-(isocyanatomethyl)oxolane (CAS 1341487-61-3) provided the title compound as a brown solid. The compound was isolated as the bis sodium salt. LCMS (ESI+): R_{f}=5.13, M+H=573.58; HRMS (ESI−): calculated for C₃₁H₂₇N₂O₉ m/z [M−H]⁻: 571.1722, observed 571.1721; ¹H NMR δ: 8.57-8.66 (m, 1H), 7.76-7.82 (m, 1H), 7.41-7.46 (m, 1H), 3.55-3.78 (m, 3H), 3.34-3.43 (m, 2H), 3.15-3.24 (m, 3H), 2.30-2.44 (m, 1H), 1.84-2.00 (m, 1H), 1.49-1.63 (m, 1H).

### Example 364—Synthesis of N-(1-methanesulfonylpiperidin-4-yl)-5-({2-[(1-methanesulfonylpiperidin-4-yl)carbamoyl]-1,3-dioxo-2,3-dihydro-1H-inden-5-yl}sulfonyl)-1,3-dioxo-2,3-dihydro-1H-indene-2-carboxamide. FC-12378

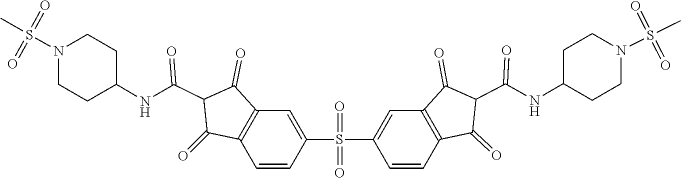

Using the procedure of Example 336, reaction of 5,5′-sulfonylbis(1H-indene-1,3(2H)-dione) (Example 11) and 4-isocyanato-1-(methylsulfonyl)piperidine (CAS 1016886-27-3) provided the title compound as a brown solid. The compound was isolated as the bis sodium salt. LCMS (ESI+): R_{f}=4.66, M+H=763.54; HRMS (ESI−): calculated for C₃₂H₃₃N₄O₁₂S₃ m/z [M−H]⁻: 761.1263, observed 761.1248; ¹H NMR δ: 8.45-8.52 (m, 1H), 8.01-8.06 (m, 1H), 7.61-7.64 (m, 1H), 7.44-7.49 (m, 1H), 3.73-3.87 (m, 1H), 3.35-3.48 (m, 3H), 2.81-2.96 (m, 6H), 1.82-1.98 (m, 2H), 1.38-1.54 (m, 2H).

### Example 365—Synthesis of N-(1-methanesulfonylpiperidin-4-yl)-5-{2-[(1-methanesulfonylpiperidin-4-yl)carbamoyl]-1,3-dioxo-2,3-dihydro-1H-indene-5-carbonyl}-1,3-dioxo-2,3-dihydro-1H-indene-2-carboxamide. FC-12379

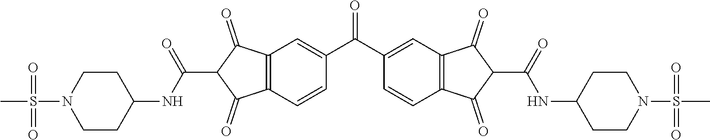

Using the procedure of Example 335, reaction of 5,5′-carbonylbis(1H-indene-1,3(2H)-dione (Example 4) and 4-isocyanato-1-(methylsulfonyl)piperidine (CAS 1016886-27-3) provided the title compound as a brown solid. The compound was isolated as the bis sodium salt. LCMS (ESI+): R_{f}=4.93, M+H=727.63; HRMS (ESI−): calculated for C₃₃H₃₃N₄O₁₁S₂ m/z [M−H]⁻: 725.1593, observed 725.1583; ¹H NMR δ: 8.55-8.62 (m, 1H), 7.77-7.82 (m, 1H), 7.52-7.56 (m, 1H), 7.42-7.46 (m, 1H), 3.76-3.90 (m, 1H), 3.38-3.51 (m, 3H), 32.83-2.98 (m, 6H), 1.85-2.00 (m, 2H), 1.38-1.59 (m, 2H).

### Example 366—Synthesis of N-(1,1-dioxo-1λ⁶-thiolan-3-yl)-5-({2-[(1,1-dioxo-1λ⁶-thiolan-3-yl)carbamoyl]-1,3-dioxo-2,3-dihydro-1H-inden-5-yl}oxy)-1,3-dioxo-2,3-dihydro-1H-indene-2-carboxamide. FC-12393

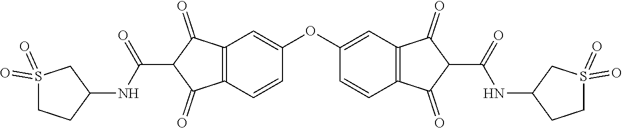 [SMILES: O=C(NC1CCS(=O)(=O)C1)C1C(=O)c2ccc(Oc3ccc4c(c3)C(=O)C(C(=O)NC3CCS(=O)(=O)C3)C4=O)cc2C1=O]

Using the procedure of Example 334, reaction of 5-(2,3-dihydro-1,3-dioxo-1H-inden-6-yloxy)-2,3-dihydro-1,3-dioxo-1H-indene (Example 7) and 3-isocyananto-tetrahydro-thiophene 1,1-dioxide (CAS 24373-66-8) provided the title compound as a brown solid. The compound was isolated as the bis sodium salt. LCMS (ESI+): R_{f}=4.32, M+H=629.48; HRMS (ESI−): calculated for C₂₈H₂₃N₂O₁₁S₂ m/z [M−H]⁻: 627.074875, observed 627.073570; ¹H NMR δ: 8.72-8.79 (m, 1H), 7.27-7.33 (m, 1H), 6.97-7.05 (m, 1H), 6.82-6.89 (m, 1H), 4.48-4.64 (m, 1H), 3.33-3.43 (m, 1H), 3.07-3.25 (m, 3H), 2.89-3.00 (m, 1H), 2.02-2.15 (m, 1H).

### Example 367—Synthesis of N-[(1,1-dioxo-1λ⁶-thiolan-3-yl)methyl]-5-[(2-{[(1,1-dioxo-1λ⁶-thiolan-3-yl)methyl]carbamoyl}-1,3-dioxo-2,3-dihydro-1H-inden-5-yl)oxy]-1,3-dioxo-2,3-dihydro-1H-indene-2-carboxamide. FC-12394

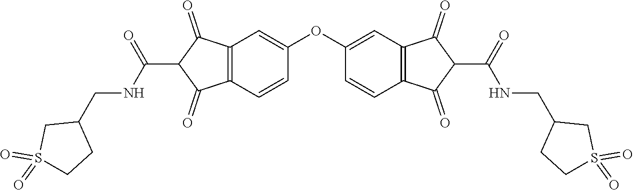 [SMILES: O=C(NCC1CCS(=O)(=O)C1)C1C(=O)c2ccc(Oc3ccc4c(c3)C(=O)C(C(=O)NCC3CCS(=O)(=O)C3)C4=O)cc2C1=O]

Using the procedure of Example 334, reaction of 5-(2,3-dihydro-1,3-dioxo-1H-inden-6-yloxy)-2,3-dihydro-1,3-dioxo-1H-indene (Example 7) and 3-(isocyanantomethyl) tetrahydrothiophene-1,1-dioxide, (CAS 2880041-1) provided the title compound as a brown solid. The compound was isolated as the bis sodium salt. LCMS (ESI+): R_{f}=4.38, M+Na=679.50; HRMS (ESI−): calculated for C₃₀H₂₇N₂O₁₁S₂ m/z [M−H]⁻: 655.1062, observed 655.1067; ¹H NMR δ: 8.57-8.67 (m, 1H), 7.26-7.35 (m, 1H), 3.97-7.07 (m, 1H), 6.82-6.88 (m, 1H), 2.93-3.21 (m, 5H), 2.69-2.83 (m, 1H), 2.09-2.25 (m, 1H), 1.69-1.89 (m, 1H).

### Example 368—Synthesis of N-(oxan-4-yl)-5-({2-[(oxan-4-yl)carbamoyl]-1,3-dioxo-2,3-dihydro-1H-inden-5-yl}sulfonyl)-1,3-dioxo-2,3-dihydro-1H-indene-2-carboxamide. FC-12422

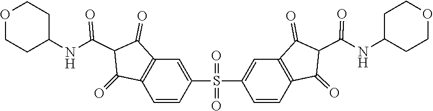 [SMILES: O=C(NC1CCOCC1)C1C(=O)c2ccc(S(=O)(=O)c3ccc4c(c3)C(=O)C(C(=O)NC3CCOCC3)C4=O)cc2C1=O]

Using the procedure of Example 336, reaction of 5,5′-sulfonylbis(1H-indene-1,3(2H)-dione) (Example 11) and 4-isocyanato-tetrahydropyran (CAS 53035-92-0) provided the title compound as a brown solid. The compound was isolated as the bis sodium salt. LCMS (ESI+): R_{f}=4.85, M+H=609.50; HRMS (ESI−): calculated for C₃₀H₂₇N₂O₁₀S m/z [M−H]⁻: 607.1392, observed 607.1386; ¹H NMR δ: 8.43-8.49 (m, 1H), 8.01-8.06 (m, 1H), 7.60-7.63 (m, 1H), 7.43-7.48 (m, 1H), 3.74-3.83 (m, 3H), 3.36-3.43 (m, 2H), 1.71-1.83 (m, 3H), 1.30-1.45 (m, 3H).

### Example 369—Synthesis of N-(1-methanesulfonylpiperidin-4-yl)-5-({2-[(1-methanesulfonylpiperidin-4-yl)carbamoyl]-1,3-dioxo-2,3-dihydro-1H-inden-5-yl}oxy)-1,3-dioxo-2,3-dihydro-1H-indene-2-carboxamide. FC-12423

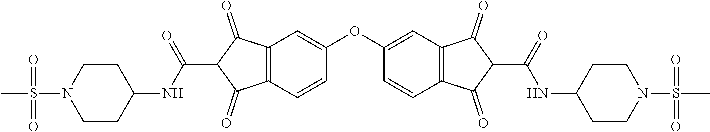

Using the procedure of Example 334, reaction of 5-(2,3-dihydro-1,3-dioxo-1H-inden-6-yloxy)-2,3-dihydro-1,3-dioxo-1H-indene (Example 7) and 4-isocyanato-1-(methylsulfonyl) piperidine, (CAS number 1016886-27-3) provided the title compound as a brown solid. The compound was isolated as the bis sodium salt. LCMS (ESI+): R_{f}=4.94, M+H=715.48; HRMS (ESI−): calculated for C₃₂H₃₃N₄O₁₁S₂ m/z [M−H]⁻: 713.1593, observed 713.1577; ¹H NMR δ: 8.50-8.59 (m, 1H), 7.24-7.33 (m, 1H), 6.80-6.87 (m, 1H), 3.71-3.89 (m, 1H), 3.37-3.48 (m, 4H), 2.79-2.97 (m, 6H), 1.82-1.97 (m, 2H), 1.35-1.55 (m, 2H).

### Example 370—Synthesis of 2-(5-{[2-(1,1-dioxo-1λ⁶-thiolane-2-carbonyl)-1,3-dioxo-2,3-dihydro-1H-inden-5-yl]oxy}-1,3-dioxo-2,3-dihydro-1H-indene-2-carbonyl)-1λ⁶-thiolane-1,1-dione. FC-12447

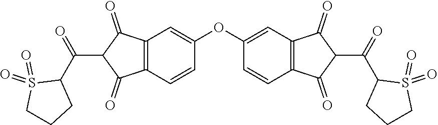 [SMILES: O=C1c2ccc(Oc3ccc4c(c3)C(=O)C(C(=O)C3CCCS3(=O)=O)C4=O)cc2C(=O)C1C(=O)C1CCCS1(=O)=O]

Using the procedure of Example 346, reaction of 5-(2,3-dihydro-1,3-dioxo-1H-inden-6-yloxy)-2,3-dihydro-1,3-dioxo-1H-indene (Example 7) and tetrahydrothiophene-3-carboxylic acid 1,1-dioxide (CAS number 80548-40-9) provided the title compound as a brown solid. The compound was isolated as the bis sodium salt. LCMS (ESI+): R_{f}=4.40, M+H=599.37; HRMS (ESI−): calculated for C₂₈H₂₁O₁₁S₂ m/z [M−H]⁻: 597.0531, observed 597.0531; ¹H NMR δ: 7.41-7.48 (m, 1H), 7.11-7.17 (m, 1H), 6.94-7.00 (m, 1H), 5.58-5.67 (m, 1H), 2.94-3.06 (m, 1H), 2.76-2.88 (m, 1H), 2.29-2.39 (m, 1H), 2.08-2.20 (m, 2H), 1.86-2.00 (m, 1H).

### Example 371—Synthesis of 2-{5-[2-(1,1-dioxo-1λ⁶-thiolane-2-carbonyl)-1,3-dioxo-2,3-dihydro-1H-indene-5-carbonyl]-1,3-dioxo-2,3-dihydro-1H-indene-2-carbonyl}-1λ⁶-thiolane-1,1-dione. FC-12448

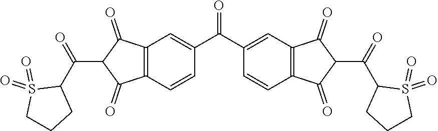 [SMILES: O=C(c1ccc2c(c1)C(=O)C(C(=O)C1CCCS1(=O)=O)C2=O)c1ccc2c(c1)C(=O)C(C(=O)C1CCCS1(=O)=O)C2=O]

Using the procedure of Example 347, reaction of 5,5′-carbonylbis(1H-indene-1,3(2H)-dione (Example 4) and tetrahydrothiophene-3-carboxylic acid 1,1-dioxide (CAS number 80548-40-9) provided the title compound as a brown solid. The compound was isolated as the bis sodium salt. LCMS (ESI+): R_{f}=4.01, M+H=611.39; HRMS (ESI−): calculated for C₂₉H₂₁O₁₁S₂ m/z [M−H]⁻: 609.0531, observed 609.0527; ¹H NMR δ: 7.85-7.89 (m, 1H), 7.66-7.69 (m, 1H), 7.55-7.59 (m, 1H), 5.61-5.67 (m, 1H), 2.96-3.08 (m, 1H), 2.79-2.89 (m, 1H), 2.38-2.43 (m, 1H), 2.07-2.23 (m, 2H), 1.87-2.02 (m, 1H).

### Example 372—Synthesis of 2-(5-{[2-(1,1-dioxo-1λ⁶-thiolane-2-carbonyl)-1,3-dioxo-2,3-dihydro-1H-inden-5-yl]sulfonyl}-1,3-dioxo-2,3-dihydro-1H-indene-2-carbonyl)-1λ⁶-thiolane-1,1-dione. FC-12449

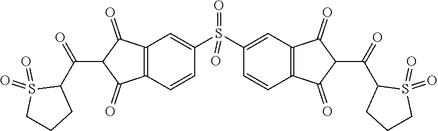 [SMILES: O=C1c2ccc(S(=O)(=O)c3ccc4c(c3)C(=O)C(C(=O)C3CCCS3(=O)=O)C4=O)cc2C(=O)C1C(=O)C1CCCS1(=O)=O]

Using the procedure of Example 348, reaction of 5,5′-sulfonylbis(1H-indene-1,3(2H)-dione) (Example 11) and tetrahydrothiophene-2-carboxylic acid 1,1-dioxide (CAS number 80548-40-9) provided the title compound as a brown solid. The compound was isolated as the bis sodium salt. LCMS (ESI+): R_{f}=3.74, M+Na=647.30; HRMS (ESI−): calculated for C₂₈H₂₁O₁₂S₃ m/z [M−H]⁻: 645.0201, observed 645.0189.

### Example 373—Synthesis of tert-butyl (3S)-3-(5-{2-[(3S)-1-[(tert-butoxy)carbonyl]pyrrolidine-3-carbonyl]-1,3-dioxo-2,3-dihydro-1H-indene-5-carbonyl}-1,3-dioxo-2,3-dihydro-1H-indene-2-carbonyl)pyrrolidine-1-carboxylate. FC-12459

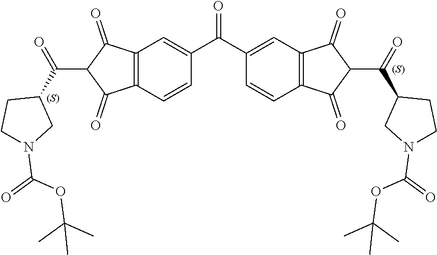

Using the procedure of Example 347, reaction of 5,5′-carbonylbis(1H-indene-1,3(2H)-dione (Example 4) and (S)-1-Boc-pyrrolidinecarboxylic acid (CAS number 140148-70-5) provided the title compound as a brown solid. The compound was isolated as the bis sodium salt. LCMS (ESI+): R_{f}=6.73, M+Na=735.66; HRMS (ESI−): calculated for C₃₉H₃₉N₂O₁₁ m/z [M−H]⁻: 711.2559, observed 711.2572; ¹H NMR δ: 7.82-7.88 (m, 1H), 7.61-7.65 (m, 1H), 7.51-7.55 (m, 1H), 4.06-4.23 (m, 1H), 1.83-2.01 (m, 2H), 1.33-1.42 (m, 9H).

### Example 374—Synthesis of tert-butyl (3S)-3-[5-({2-[(3S)-1-[(tert-butoxy)carbonyl]pyrrolidine-3-carbonyl]-1,3-dioxo-2,3-dihydro-1H-inden-5-yl}sulfonyl)-1,3-dioxo-2,3-dihydro-1H-indene-2-carbonyl]pyrrolidine-1-carboxylate. FC-12463

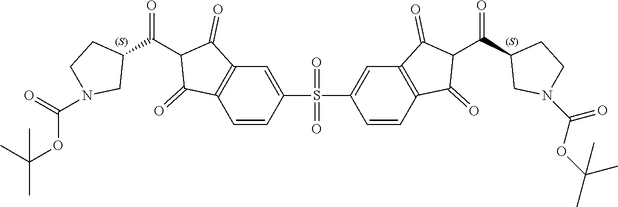

Using the procedure of Example 348, reaction of 5,5′-sulfonylbis(1H-indene-1,3(2H)-dione) (Example 11) and (S)-1-Boc-pyrrolidinecarboxylic acid (CAS number 140148-70-5) provided the title compound as a brown solid. The compound was isolated as the bis sodium salt. LCMS (ESI+): R_{f}=6.41, M+Na=771.57; HRMS (ESI−): calculated for C₃₈H₃₉N₂O₁₂S m/z [M−H]⁻: 747.2229, observed 747.2250; ¹H NMR δ: 8.05-8.11 (m, 1H), 7.73-7.76 (m, 1H), 7.53-7.58 (m, 1H), 3.99-4.15 (m, 1H), 3.14-3.26 (m, 3H), 1.79-1.98 (m, 3H), 1.31-1.40 (m, 9H).

### Example 375—Synthesis of tert-butyl (3R)-3-(5-{2-[(3R)-1-[(tert-butoxy)carbonyl]pyrrolidine-3-carbonyl]-1,3-dioxo-2,3-dihydro-1H-indene-5-carbonyl}-1,3-dioxo-2,3-dihydro-1H-indene-2-carbonyl)pyrrolidine-1-carboxylate. FC-12507

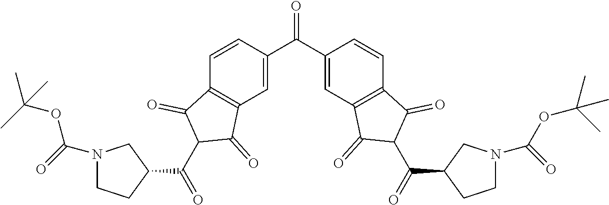 [SMILES: CC(C)(C)OC(=O)N1CC[C@@H](C(=O)C2C(=O)c3ccc(C(=O)c4ccc5c(c4)C(=O)C(C(=O)[C@@H]4CCN(C(=O)OC(C)(C)C)C4)C5=O)cc3C2=O)C1]

Using the procedure of Example 347, reaction of 5,5′-carbonylbis(1H-indene-1,3(2H)-dione (Example 4) and (3R)-1-(tert-butoxycarbonyl)-3-pyrrolidinecarboxylic acid (CAS number 72925-16-7) provided the title compound as a brown solid. The compound was isolated as the bis sodium salt. LCMS (ESI+): R_{f}=6.73, M+Na=735.66; ¹H NMR δ: 7.95-8.01 (m, 1H), 7.80 (s, 1H), 7.74 (d, J=7.62 Hz, 1H), 4.18 (m, 1H), 3.20-3.41 (m, 4H), 1.90-2.14 (m, 2H), 1.29-1.45 (s, 9H).

### Example 376—Synthesis of tert-butyl (3R)-3-[5-({2-[(3R)-1-[(tert-butoxy)carbonyl]pyrrolidine-3-carbonyl]-1,3-dioxo-2,3-dihydro-1H-inden-5-yl}sulfonyl)-1,3-dioxo-2,3-dihydro-1H-indene-2-carbonyl]pyrrolidine-1-carboxylate. FC-12508

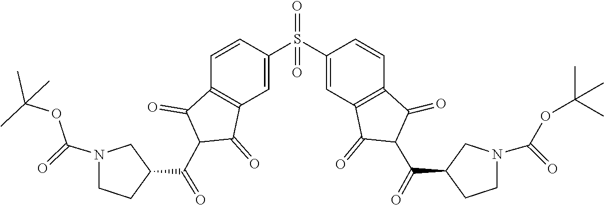 [SMILES: CC(C)(C)OC(=O)N1CC[C@@H](C(=O)C2C(=O)c3ccc(S(=O)(=O)c4ccc5c(c4)C(=O)C(C(=O)[C@@H]4CCN(C(=O)OC(C)(C)C)C4)C5=O)cc3C2=O)C1]

Using the procedure of Example 348, reaction of 5,5′-sulfonylbis(1H-indene-1,3(2H)-dione) (Example 11) and (3R)-1-(tert-butoxycarbonyl)-3-pyrrolidinecarboxylic acid, CAS number 72925-16-7 provided the title compound as a brown solid. The compound was isolated as the bis sodium salt. LCMS (ESI+): R_{f}=6.37, M+Na=771.57; ¹H NMR δ: 8.08 (dd, J=7.62, 1.76 Hz, 1H), 7.74 (d, J=1.76 Hz, 1H), 7.55 (d, J=7.62 Hz, 1H), 4.00-4.14 (m, 1H), 3.14-3.25 (m, 4H), 1.88-1.92 (m, 2H), 1.35 (br. s., 9H).

### Example 377—Synthesis of 2-(2-methoxyacetyl)-5-{[2-(2-methoxyacetyl)-1,3-dioxo-2,3-dihydro-1H-inden-5-yl]sulfonyl}-2,3-dihydro-1H-indene-1,3-dione. FC-12777

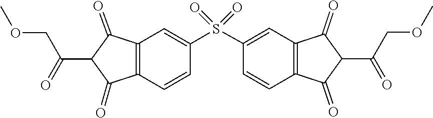

Using the procedure of Example 348, reaction of 5,5′-sulfonylbis(1H-indene-1,3(2H)-dione) (Example 11) and methoxy acetic acid (CAS number 625-45-6) provided the title compound as a brown solid. The compound was isolated as the bis sodium salt. LCMS (ESI+): R_{f}=2.92, M+H=499.03; HRMS (ESI−): calculated for C₂₄H₁₇O₁₀S m/z [M−H]⁻: 497.0548, observed 497.0555; ¹H NMR δ: 8.09 (d, J=6.45 Hz, 1H), 7.74 (br. s. 1H), 7.55 (d, J=7.62 Hz, 1H), 4.37 (br. s. 2H), 3.26 (s, 3H).

### Example 378—Synthesis of 5-({1,3-dioxo-2-[2-(propan-2-yloxy)acetyl]-2,3-dihydro-1H-inden-5-yl}oxy)-2-[2-(propan-2-yloxy)acetyl]-2,3-dihydro-1H-indene-1,3-dione. FC-12796

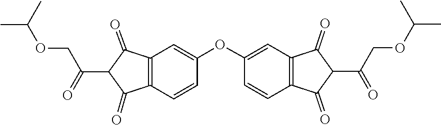 [SMILES: CC(C)OCC(=O)C1C(=O)c2ccc(Oc3ccc4c(c3)C(=O)C(C(=O)COC(C)C)C4=O)cc2C1=O]

Using the procedure of Example 346, reaction of 5-(2,3-dihydro-1,3-dioxo-1H-inden-6-yloxy)-2,3-dihydro-1,3-dioxo-1H-indene (Example 7) and isopropoxyacetic acid (CAS number 33445-07-7) provided the title compound as a brown solid. The compound was isolated as the bis sodium salt. LCMS (ESI+): R_{f}=6.27, M+H=507.36; HRMS (ESI−): calculated for C₂₈H₂₅O₉ m/z [M−H]⁻: 505.1504, observed 505.1506; ¹H NMR δ: 7.37 (d, J=7.62 Hz, 1H), 7.06-7.11 (m, 1H), 6.88 (d, J=2.34 Hz, 1H), 4.42 (s, 2H), 3.58 (quint, J=6.01, 1H), 1.07 (d, J=6.45 Hz, 6H).

### Example 379—Synthesis of 5-({1,3-dioxo-2-[2-(propan-2-yloxy)acetyl]-2,3-dihydro-1H-inden-5-yl}sulfonyl)-2-[2-(propan-2-yloxy)acetyl]-2,3-dihydro-1H-indene-1,3-dione. FC-12797

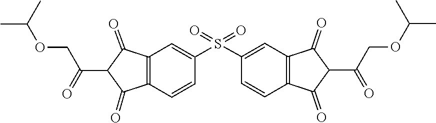 [SMILES: CC(C)OCC(=O)C1C(=O)c2ccc(S(=O)(=O)c3ccc4c(c3)C(=O)C(C(=O)COC(C)C)C4=O)cc2C1=O]

Using the procedure of Example 348, reaction of 5,5′-sulfonylbis(1H-indene-1,3(2H)-dione) (Example 11) and isopropoxyacetic acid (CAS number 33445-07-7) provided the title compound as a brown solid. The compound was isolated as the bis sodium salt. LCMS (ESI+): R_{f}=4.56, M+H=555.36; HRMS (ESI−): calculated for C₂₈H₂₅O₁₀S m/z [M−H]⁻: 553.1174, observed 553.1159; ¹H NMR δ: 8.07 (d, J=7.03 Hz, 1H), 7.72 (s, 1H), 7.53 (d, J=8.21 Hz, 1H), 4.41 (s, 2H), 3.56 (quint, J=6.15 Hz, 1H), 1.07 (d, J=5.86 Hz, 6H).

### Example 380—Synthesis of 5-{1,3-dioxo-2-[2-(propan-2-yloxy)acetyl]-2,3-dihydro-1H-indene-5-carbonyl}-2-[2-(propan-2-yloxy)acetyl]-2,3-dihydro-1H-indene-1,3-dione. FC-12798

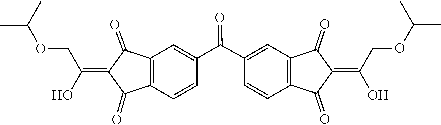 [SMILES: CC(C)OC/C(O)=C1/C(=O)c2ccc(C(=O)c3ccc4c(c3)C(=O)/C(=C(/O)COC(C)C)C4=O)cc2C1=O]

Using the procedure of Example 347, reaction of 5,5′-carbonylbis(1H-indene-1,3(2H)-dione (Example 4) and isopropoxyacetic acid (CAS number 33445-07-7) provided the title compound as a brown solid. The compound was isolated as the bis sodium salt. LCMS (ESI+): R_{f}=5.99, M+H=519.38; HRMS (ESI−): calculated for C₂₉H₂₅O₉ m/z [M−H]⁻: 517.1504, observed 517.1516; ¹H NMR δ: 7.83-7.89 (m, 1H), 7.63 (m, 1H), 7.54 (d, J=7.62 Hz, 1H), 4.48 (s, 2H), 3.60 (quint, J=6.01, 1H), 1.10 (d, J=5.86 Hz, 6H).

### Example 381—Synthesis of N-[2-(2-methoxyethoxy)ethyl]-5-[2-(2-{[2-(2-methoxyethoxy)ethyl]carbamoyl}-1,3-dioxo-2,3-dihydro-1H-inden-5-yl)ethynyl]-1,3-dioxo-2,3-dihydro-1H-indene-2-carboxamide. FC-12834

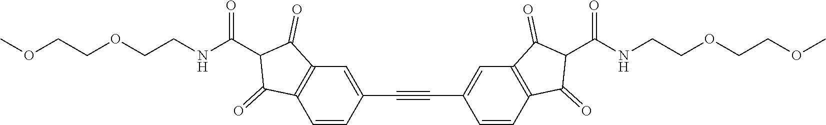

Using the procedure of Example 300, reaction of 5-[2-(1,3-dioxo-2,3-dihydro-1H-inden-5-yl)ethynyl]-2,3-dihydro-1H-indene-1,3-dione (Example 196) and 1-isocyanato-2-(2-methoxyethoxy)ethane (CAS number 90426-82-7) provided the title compound as a brown solid. LCMS (ESI⁺): RT=5.55 min, (M+H)⁺=605.5; HRMS (ESI⁻) Calcd for C₃₂H₃₁N₂O₁₀: 603.198419; Observed: 603.198016; ¹H NMR CDCl₃ δ: 8.25-8.07 (m, 1H), 7.77 (s, 1H), 7.74-7.67 (m, 1H), 7.66-7.58 (m, 1H), 4.30-3.79 (m, 1H), 3.73-3.64 (m, 6H), 3.63-3.55 (m, 2H), 3.42 (s, 3H).

### Example 382—Synthesis of 2-[3-(2-methoxyethoxy)propanoyl]-5-{2-[3-(2-methoxyethoxy)propanoyl]-1,3-dioxo-2,3-dihydro-1H-indene-5-carbonyl}-2,3-dihydro-1H-indene-1,3-dione. FC-12839

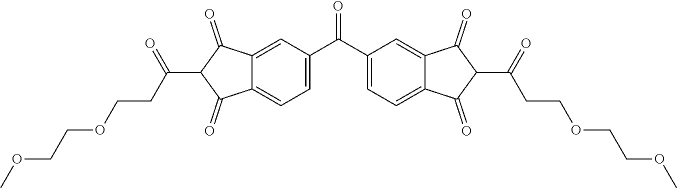

Using the procedure of Example 347, reaction of 5,5′-carbonylbis(1H-indene-1,3(2H)-dione (Example 4) and 3-(2-methoxyethoxy)propanoic acid (CAS number 149577-05-9) provided the title compound as a brown solid. LCMS (ESI+): RT=4.82 min, (M+H)=579.45; HRMS (ESI−): Predicted: 577.1715, found: 577.1712; ¹H NMR (METHANOL-d₄) δ=8.23 (dd, J=1.2, 7.6 Hz, 2H), 8.17 (s, 2H), 8.10 (d, J=7.6 Hz, 1H), 8.00-7.90 (m, 2H), 6.99 (d, J=7.6 Hz, 1H), 3.89 (t, J=6.4 Hz, 4H), 3.68-3.61 (m, 4H), 3.53 (dd, J=3.5, 5.9 Hz, 4H), 3.34 (s, 3H), 3.29-3.22 (m, 7H).

### Example 383—Synthesis of 2-[3-(2-methoxyethoxy)propanoyl]-5-({2-[3-(2-methoxyethoxy)propanoyl]-1,3-dioxo-2,3-dihydro-1H-inden-5-yl}oxy)-2,3-dihydro-1H-indene-1,3-dione. FC-12840

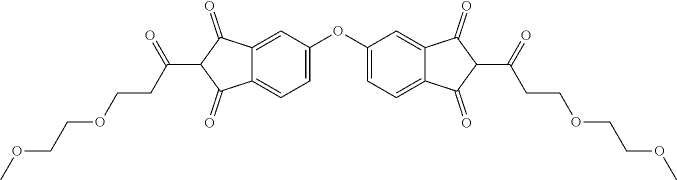

Using the procedure of Example 346, reaction of 5-(2,3-dihydro-1,3-dioxo-1H-inden-6-yloxy)-2,3-dihydro-1,3-dioxo-1H-indene (Example 7) and 3-(2-methoxyethoxy)propanoic acid (CAS number 149577-05-9) provided the title compound as a brown solid. LCMS (ESI+): RT=5.93 min, (M+H)=567.55; HRMS (ESI−): Predicted: 565.1715, found: 565.1706; ¹H NMR (METHANOL-d₄) 5=7.91 (d, J=8.2 Hz, 2H), 7.55-7.43 (m, 4H), 3.88 (t, J=6.4 Hz, 4H), 3.66-3.60 (m, 4H), 3.54-3.47 (m, 4H), 3.26 (s, 6H), 2.65 (s, 2H).

### Example 384—Synthesis of N-{2-[2-(2-methoxyethoxy)ethoxy]ethyl}-5-{[2-({2-[2-(2-methoxyethoxy)ethoxy]ethyl}carbamoyl)-1,3-dioxo-2,3-dihydro-1H-inden-5-yl]oxy}-1,3-dioxo-2,3-dihydro-1H-indene-2-carboxamide. FC-12841

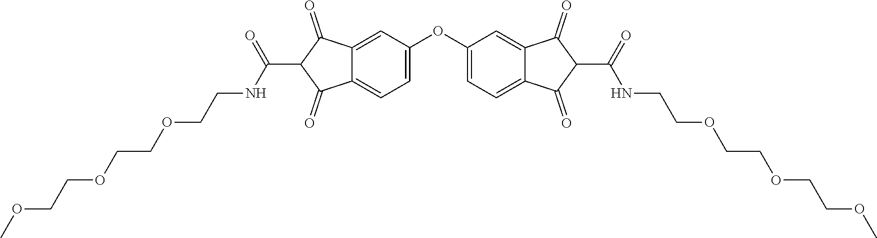

1) To a solution of triphosgene (1.36 g, 4.5 mmol) in CH₂Cl₂ (13.8 ml) under nitrogen at 0° C. was added pyridine (2.07 ml, 25.7 mmol). After stirring 20 min, a solution of 2-(2-(2-methoxyethoxy)ethoxy) ethanamine (CAS reg. no: 74654-07-2, 734 mg, 4.5 mmol) in CH₂Cl₂ (13.8 ml) was added dropwise to this mixture. The reaction was warmed to ambient temperature and stirred 45 min. The reaction was quenched by the addition of water (35 ml) and the organic phase was extracted (2×) with chloroform (35 ml), and dried over sodium sulfate. The solvent was removed in vacuo to provide crude 1-[2-(2-isocyanato-ethoxy)-ethoxy]-2-methoxy-ethane which was used as-is in the next step.

2) Using the procedure of Example 334, reaction of 5-(2,3-dihydro-1,3-dioxo-1H-inden-6-yloxy)-2,3-dihydro-1,3-dioxo-1H-indene (Example 7) and 1-[2-(2-isocyanato-ethoxy)-ethoxy]-2-methoxy-ethane from step 1 provided the title compound as a brown solid. LCMS (ESI+): RT=4.87 min, (M+H)=685.51; HRMS (ESI−): Predicted: 683.2458, found: 683.2447; ¹H NMR (METHANOL-d₄) δ=7.58 (d, J=8.2 Hz, 2H), 7.30-7.20 (m, 4H), 3.71-3.60 (m, 24H), 3.55-3.47 (m, 6H).

### Example 385—Synthesis of tert-butyl N-[4-(5-{[2-(4-{[(tert-butoxy)carbonyl]amino}butanoyl)-1,3-dioxo-2,3-dihydro-1H-inden-5-yl]oxy}-1,3-dioxo-2,3-dihydro-1H-inden-2-yl)-4-oxobutyl]carbamate. FC-12858

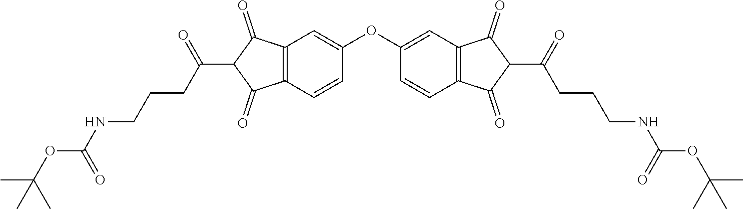 [SMILES: CC(C)(C)OC(=O)NCCCC(=O)C1C(=O)c2ccc(Oc3ccc4c(c3)C(=O)C(C(=O)CCCNC(=O)OC(C)(C)C)C4=O)cc2C1=O]

Using the procedure of Example 346, reaction of 5-(2,3-dihydro-1,3-dioxo-1H-inden-6-yloxy)-2,3-dihydro-1,3-dioxo-1H-indene (Example 7) and boc-4-aminobutanoic acid (CAS number 57294-38-9) provided the title compound as a brown solid. The compound was isolated as the bis sodium salt. LCMS (ESI+): R_{f}=6.76, M+Na=699.48; HRMS (ESI−): calculated for C₃₆H₃₉N₂O₁₁ m/z [M−H]⁻: 675.2559, observed 675.2560; ¹H NMR δ: 7.36 (d, J=8.21 Hz, 1H), 7.04-7.10 (m, 1H), 6.87 (d, J=2.34 Hz, 1H), 2.83-2.94 (m, 2H), 2.65 (t, J=7.33 Hz, 2H), 1.49-1.60 (m, 2H), 1.35 (s, 9H).

### Example 386—Synthesis of tert-butyl N-(4-{5-[2-(4-{[(tert-butoxy)carbonyl]amino}butanoyl)-1,3-dioxo-2,3-dihydro-1H-indene-5-carbonyl]-1,3-dioxo-2,3-dihydro-1H-inden-2-yl}-4-oxobutyl)carbamate. FC-12859

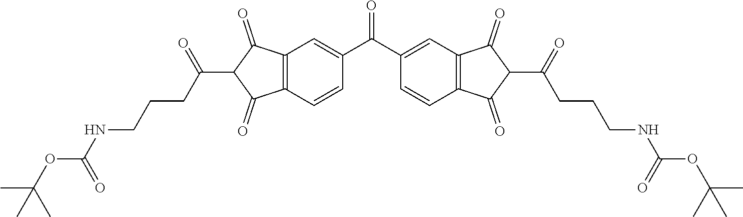 [SMILES: CC(C)(C)OC(=O)NCCCC(=O)C1C(=O)c2ccc(C(=O)c3ccc4c(c3)C(=O)C(C(=O)CCCNC(=O)OC(C)(C)C)C4=O)cc2C1=O]

Using the procedure of Example 347, reaction of 5,5′-carbonylbis(1H-indene-1,3(2H)-dione (Example 4) and boc-4-aminobutanoic acid (CAS number 57294-38-9) provided the title compound as a brown solid. The compound was isolated as the bis sodium salt. LCMS (ESI+): R_{f}=6.53, M+H=711.49; HRMS (ESI−): calculated for C₃₇H₃₉N₂O₁₁ m/z [M−H]⁻: 687.2559, observed 687.2542; ¹H NMR δ: 7.83 (dd, J=7.33, 1.47 Hz, 1H), 7.61 (s, 1H), 7.51 (d, J=7.62 Hz, 1H), 6.75 (br. s. 1H), 2.84-2.98 (m, 2H), 2.70 (t, J=7.62 Hz, 2H), 1.51-1.68 (m, 2H), 1.28-1.42 (s, 9H).

### Example 387—Synthesis of tert-butyl N-[4-(5-{[2-(4-{[(tert-butoxy)carbonyl]amino}butanoyl)-1,3-dioxo-2,3-dihydro-1H-inden-5-yl]sulfonyl}-1,3-dioxo-2,3-dihydro-1H-inden-2-yl)-4-oxobutyl]carbamate. FC-12860

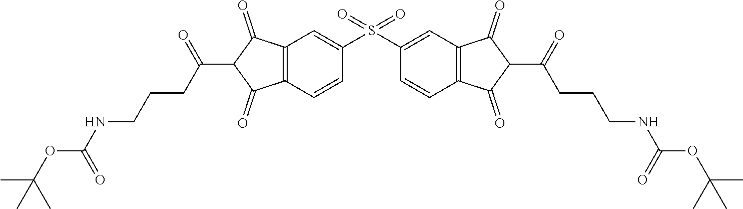 [SMILES: CC(C)(C)OC(=O)NCCCC(=O)C1C(=O)c2ccc(S(=O)(=O)c3ccc4c(c3)C(=O)C(C(=O)CCCNC(=O)OC(C)(C)C)C4=O)cc2C1=O]

Using the procedure of Example 348, reaction of 5,5′-sulfonylbis(1H-indene-1,3(2H)-dione) (Example 11) and boc-4-aminobutanoic acid (CAS number 57294-38-9) provided the title compound as a brown solid. The compound was isolated as the bis sodium salt. LCMS (ESI+): R_{f}=6.17, M+Na=747.40; HRMS (ESI−): calculated for C₃₆H₃₉N₂O₁₂S m/z [M−H]⁻: 723.2229, observed 723.2236; ¹H NMR δ: 8.06 (dd, J=7.62, 1.76 Hz, 1H), 7.72 (s, 1H), 7.53 (d, J=7.62 Hz, 1H), 6.72 (br. s. 1H), 2.87 (d, J=7.03 Hz, 2H), 2.65 (t, J=7.33 Hz, 2H), 1.56 (d, J=7.03 Hz, 2H), 1.34 (s, 9H).

### Example 388—Synthesis of N-{2-[2-(2-methoxyethoxy)ethoxy]ethyl}-5-[2-({2-[2-(2-methoxyethoxy)ethoxy]ethyl}carbamoyl)-1,3-dioxo-2,3-dihydro-1H-indene-5-carbonyl]-1,3-dioxo-2,3-dihydro-1H-indene-2-carboxamide. FC-12873

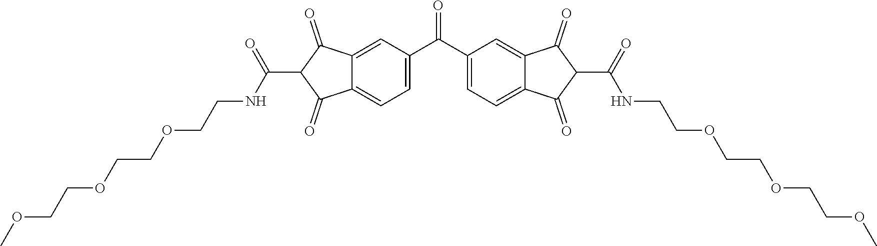

Using the procedure of Example 335, reaction of 5,5′-carbonylbis(1H-indene-1,3(2H)-dione (Example 4) and 1-[2-(2-isocyanato-ethoxy)-ethoxy]-2-methoxy-ethane (Example 384, step 1) provided the title compound as a brown solid. LCMS (ESI+): RT=4.55 min, (M+H)=697.62; HRMS (ESI−): Predicted: 695.2458, found: 695.2438; ¹H NMR (300 MHz, METHANOL-d₄) 5=7.90 (br d, J=6.4 Hz, 2H), 7.74 (br s, 2H), 7.56 (br d, J=7.6 Hz, 2H), 3.73-3.46 (m, 24H), 3.38-3.34 (m, 2H), 2.70 (s, 6H).

### Example 389—Synthesis of N-cyclobutyl-5-{[2-(cyclobutylcarbamoyl)-1,3-dioxo-2,3-dihydro-1H-inden-5-yl]oxy}-1,3-dioxo-2,3-dihydro-1H-indene-2-carboxamide. FC-12889

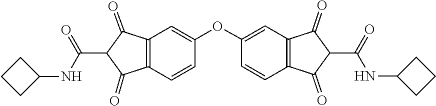 [SMILES: O=C(NC1CCC1)C1C(=O)c2ccc(Oc3ccc4c(c3)C(=O)C(C(=O)NC3CCC3)C4=O)cc2C1=O]

Using the procedure of Example 334, reaction of 5-(2,3-dihydro-1,3-dioxo-1H-inden-6-yloxy)-2,3-dihydro-1,3-dioxo-1H-indene (Example 7) and cyclobutyl isocyanate (CAS 5811-25-6) provided the title compound as a brown solid. The compound was isolated as the bis sodium salt. LCMS (ESI+): R_{f}=7.41, M+H=501.49; HRMS (ESI−): calculated for C₂₈H₂₃N₂O₇ m/z [M−H]⁻: 499.151075, observed 499.152171; ¹H NMR δ: 8.65 (d, J=8.21 Hz, 1H), 7.26 (d, J=8.21 Hz, 1H), 6.99 (dd, J=8.21, 2.34 Hz, 1H). 6.81 (d, J=2.34 Hz, 1H), 4.32 (s, 2H), 2.11-2.38 (m, 3H), 1.80 (s, 2H), 1.60 (d, J=8.21 Hz, 2H).

### Example 390—Synthesis of tert-butyl N-{2-[5-({2-[2-(11,11-dimethyl-9-oxo-2,5,10-trioxa-8-azadodecan-8-yl)acetyl]-1,3-dioxo-2,3-dihydro-1H-inden-5-yl}oxy)-1,3-dioxo-2,3-dihydro-1H-inden-2-yl]-2-oxoethyl}-N-[2-(2-methoxyethoxy)ethyl]carbamate. FC-12894

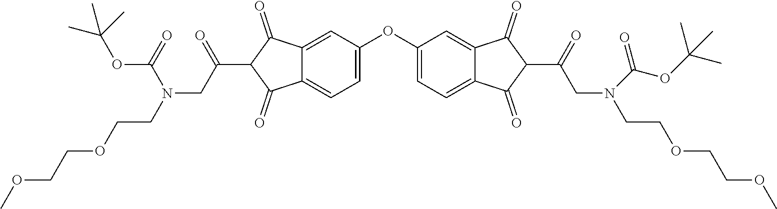

1) To a solution of benzyl bromoacetate (CAS reg no: 5437-45-6, 3.44 g, 15 mmol) in CH₂Cl₂ (19.5 ml) at 0° C. was added triethylamine (4.18 ml, 30 mmol). After stirring for 20 min, a solution of 2-(2-methoxyethoxy) enamine (CAS reg no: 31576-51-9, 3.73 ml, 30 mmol) in CH₂Cl₂ (13 ml) was added. The reaction was warmed to ambient temperature and for 5 hr. Water (25 ml) was added and the layers were separated. The organic phase was extracted (2×) with CH₂Cl₂ (20 ml) and the combined organic layers were dried over magnesium sulfate. The solvent was removed in vacuo and the residue was purified via silica gel chromatography using a 0-10% MeOH/CH₂Cl₂ gradient buffered with ammonium hydroxide to give N-[2-(2-methoxyethoxy)ethyl]glycine benzyl ester (1.77 g, 44%). LCMS (ESI+): RT=2.90 min, (M+H)=268.36

2) To a solution of N-[2-(2-methoxyethoxy)ethyl]glycine (1.7 g, 6.5 mmol) in CH₂Cl₂ (25.6 ml) was added di-tert-butyl decarbonate (CAS reg no: 24424-99-5, 2.1 g, 9.6 mmol) and i-Pr₂NEt (2.23 ml, 2.0 mmol). The reaction mixture was stirred at ambient temperature overnight. The reaction was diluted with water (20 ml) and the layers were separated. The organic phase was extracted with CH₂Cl₂ (25 ml) and the combined organic layers were dried over sodium sulfate. The solvent was removed in vacuo and the residue was purified via silica gel chromatography using a 0-100% EtOAc/Hexanes gradient to give N-(tert-butoxycarbonyl)-N-[2-(2-methoxyethoxy)ethyl]glycine benzyl ester (2.25 g, 96%). LCMS (ESI+): RT=4.83 min, (M+Na)=390.44; ¹H NMR (CHLOROFORM-d) 5=7.35 (d, J=2.9 Hz, 5H), 5.16 (d, J=2.9 Hz, 2H), 4.13 (s, 2H), 4.06 (s, 2H), 3.63-3.42 (m, 6H), 3.36-3.33 (m, 3H), 1.46 (s, 4H), 1.36 (s, 5H).

3) To a solution of N-(tert-butoxycarbonyl)-N-[2-(2-methoxyethoxy)ethyl]glycine benzyl ester (2.23 g, 6 mmol) in MeOH (19 ml) was added 10% palladium on activated carbon (235 mg) and 5 drops of acetic acid. The mixture was stirred vigorously and placed under vacuum and backfilled with hydrogen gas (3×). The reaction was stirred under hydrogen gas overnight at which point no starting material was present by LC/MS. The reaction was then filtered to remove palladium catalyst and the solvent was removed in vacuo to give N-(tert-butoxy carbonyl)-N-[2-(2-methoxyethoxy)ethyl]glycine (1.6 g, 96%) as a yellow oil. LCMS (ESI+): RT=3.39 min, (M+Na)=300.35; ¹H NMR (CHLOROFORM-d) δ=4.12 (s, 2H), 3.78-3.58 (m, 2H), 3.57-3.51 (m, 4H), 3.49 (s, 2H), 3.37 (s, 3H), 1.45 (s, 9H)

Using the procedure of Example 346, reaction of 5-(2,3-dihydro-1,3-dioxo-1H-inden-6-yloxy)-2,3-dihydro-1,3-dioxo-1H-indene (Example 7) and N-(tert-butoxy carbonyl)-N-[2-(2-methoxyethoxy)ethyl]glycine from step 3 provided the title compound as a brown solid. LCMS (ESI+): RT=5.67 min, (M−Boc+H)=724.69, (M+Na)=847.78; HRMS (ESI−): Predicted: 823.3295, found: 823.3326; ¹H NMR (METHANOL-d₄) δ=7.71-7.52 (m, 2H), 7.46-7.12 (m, 4H), 4.00-3.93 (m, 2H), 3.60-3.39 (m, 20H), 3.35-3.32 (m, 6H), 1.44 (s, 9H), 1.41 (s, 9H).

### Example 391—Synthesis of tert-butyl N-[2-(5-{2-[2-(11,11-dimethyl-9-oxo-2,5,10-trioxa-8-azadodecan-8-yl)acetyl]-1,3-dioxo-2,3-dihydro-1H-indene-5-carbonyl}-1,3-dioxo-2,3-dihydro-1H-inden-2-yl)-2-oxoethyl]-N-[2-(2-methoxyethoxy)ethyl]carbamate. FC-12916

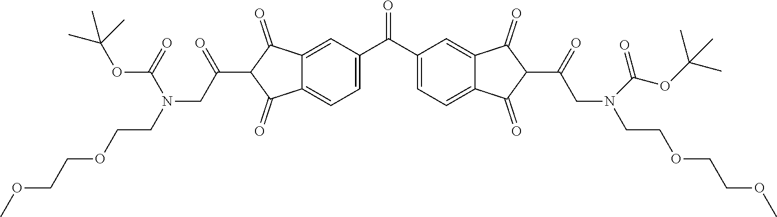

Using the procedure of Example 347, reaction of 5,5′-carbonylbis(1H-indene-1,3(2H)-dione (Example 4) and N-(tert-butoxy carbonyl)-N-[2-(2-methoxyethoxy)ethyl]glycine (Example 390, step 3) provided the title compound as a brown solid. LCMS (ESI+): RT=5.50 min, (M−Boc+H)=737.59, (M+Na)=859.61; HRMS (ESI−): Predicted: 835.3295, found: 835.3324; ¹H NMR (METHANOL-d₄) δ=8.16 (d, J=8.2 Hz, 2H), 8.11 (s, 2H), 7.92 (d, J=7.6 Hz, 2H), 4.81 (s, 4H), 3.67-3.42 (m, 16H), 3.29-3.27 (m, 6H), 1.49 (s, 9H), 1.38 (s, 9H).

### Example 392—Synthesis of tert-butyl N-{2-[5-({2-[2-(11,11-dimethyl-9-oxo-2,5,10-trioxa-8-azadodecan-8-yl)acetyl]-1,3-dioxo-2,3-dihydro-1H-inden-5-yl}sulfonyl)-1,3-dioxo-2,3-dihydro-1H-inden-2-yl]-2-oxoethyl}-N-[2-(2-methoxyethoxy)ethyl]carbamate. FC-12917

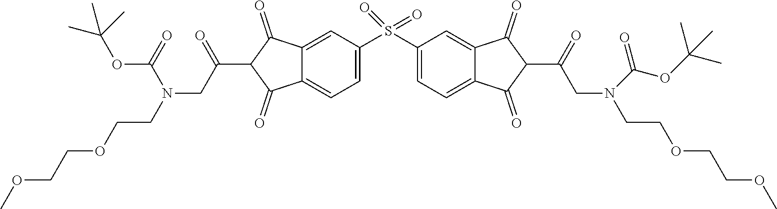

Using the procedure of Example 348, reaction of 5,5′-sulfonylbis(1H-indene-1,3(2H)-dione) (Example 11) and N-(tert-butoxy carbonyl)-N-[2-(2-methoxyethoxy)ethyl]glycine (Example 390, step 3) provided the title compound as a brown solid. LCMS (ESI+): RT=4.90 min, (M−Boc+H)=773.50, (M+Na)=895.80; HRMS (ESI−): Predicted: 871.2965, found: 871.2974; ¹H NMR (METHANOL-d₄) δ=8.39-8.32 (m, 2H), 8.27 (s, 2H), 8.10 (d, J=7.6 Hz, 1H), 7.91 (d, J=8.2 Hz, 2H), 6.98 (d, J=7.6 Hz, 1H), 4.76 (s, 4H), 3.63-3.41 (m, 16H), 3.27-3.21 (m, 6H), 1.46 (s, 8H), 1.35 (s, 11H).

### Example 393—Synthesis of 2-(2-{[2-(2-methoxyethoxy)ethyl]amino}acetyl)-5-[2-(2-{[2-(2-methoxyethoxy)ethyl]amino}acetyl)-1,3-dioxo-2,3-dihydro-1H-indene-5-carbonyl]-2,3-dihydro-1H-indene-1,3-dione. FC-12921

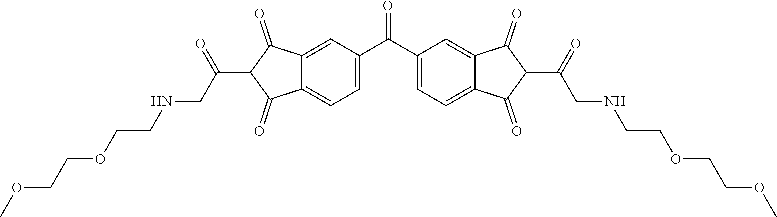

Using the procedure of Example 357, the tbutylcarboxyl moiety of tert-butyl N-[2-(5-{2-[2-(11,11-dimethyl-9-oxo-2,5,10-trioxa-8-azadodecan-8-yl)acetyl]-1,3-dioxo-2,3-dihydro-1H-indene-5-carbonyl}-1,3-dioxo-2,3-dihydro-1H-inden-2-yl)-2-oxoethyl]-N-[2-(2-methoxyethoxy)ethyl]carbamate (Example 391) was removed with trifluoroacetic acid and the compound was isolated as the bis trifluoroacetic acid salt. LCMS (ESI+): RT=2.83 min, (M+H)=637.49; HRMS (ESI+): Predicted: 637.2392, found: 637.2397; ¹H NMR (METHANOL-d₄) 5=8.10 (d, J=7.6 Hz, 1H), 7.97 (dd, J=1.8, 7.6 Hz, 2H), 7.89 (s, 2H), 7.68 (d, J=7.6 Hz, 2H), 6.99 (d, J=8.2 Hz, 1H), 4.36 (s, 4H), 3.84-3.77 (m, 4H), 3.72-3.66 (m, 6H), 3.63-3.56 (m, 6H), 3.42-3.37 (m, 6H).

### Example 394—Synthesis of 2-(2-{[2-(2-methoxyethoxy)ethyl]amino}acetyl)-5-{[2-(2-{[2-(2-methoxyethoxy)ethyl]amino}acetyl)-1,3-dioxo-2,3-dihydro-1H-inden-5-yl]oxy}-2,3-dihydro-1H-indene-1,3-dione. FC-12922

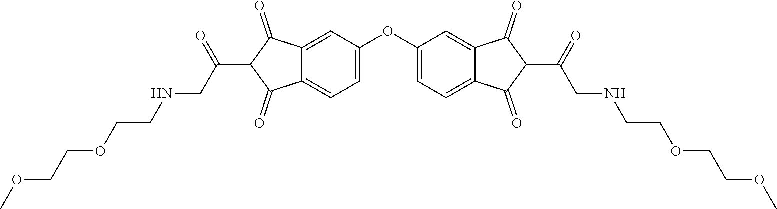

Using the procedure of Example 357, the tbutylcarboxyl moiety of tert-butyl N-{2-[5-({2-[2-(11,11-dimethyl-9-oxo-2,5,10-trioxa-8-azadodecan-8-yl)acetyl]-1,3-dioxo-2,3-dihydro-1H-inden-5-yl}oxy)-1,3-dioxo-2,3-dihydro-1H-inden-2-yl]-2-oxoethyl}-N-[2-(2-methoxyethoxy)ethyl]carbamate (Example 390) was removed with trifluoroacetic acid and the compound was isolated as the bis trifluoroacetic acid salt. LCMS (ESI+): RT=2.83 min, (M+H)=637.49; HRMS (ESI+): Predicted: 637.2392, found: 637.2397; ¹H NMR (METHANOL-d₄) 5=8.10 (d, J=7.6 Hz, 1H), 7.97 (dd, J=1.8, 7.6 Hz, 2H), 7.89 (s, 2H), 7.68 (d, J=7.6 Hz, 2H), 6.99 (d, J=8.2 Hz, 1H), 4.36 (s, 4H), 3.84-3.77 (m, 4H), 3.72-3.66 (m, 6H), 3.63-3.56 (m, 6H), 3.42-3.37 (m, 6H).

### Example 395—Synthesis of 2-[2-(2-methoxyethoxy)acetyl]-5-{2-[2-(2-methoxyethoxy)acetyl]-1,3-dioxo-2,3-dihydro-1H-indene-5-carbonyl}-2,3-dihydro-1H-indene-1,3-dione. FC-12944

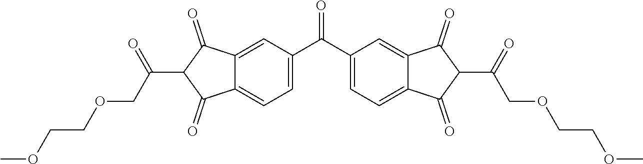

Using the procedure of Example 347, reaction of 5,5′-carbonylbis(1H-indene-1,3(2H)-dione (Example 4) and 2-(2-methoxyethoxy)acetic acid (CAS number 16024-56-9) provided the title compound as a brown solid. LCMS (ESI+): RT=3.42 min, (M+H)=551.66; ¹H NMR δ: 7.91-7.80 (m, 2H), 7.72-7.49 (m, 4H), 4.58-4.44 (m, 4H), 3.64-3.53 (m, 8H), 1.78-1.70 (m, 6H).

### Example 396—Synthesis of 2-[2-(2-methoxyethoxy)acetyl]-5-({2-[2-(2-methoxyethoxy)acetyl]-1,3-dioxo-2,3-dihydro-1H-inden-5-yl}oxy)-2,3-dihydro-1H-indene-1,3-dione. FC-12495

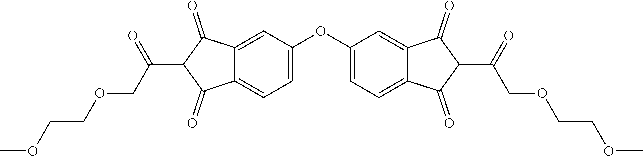

Using the procedure of Example 346, reaction of 5-(2,3-dihydro-1,3-dioxo-1H-inden-6-yloxy)-2,3-dihydro-1,3-dioxo-1H-indene (Example 7) and 2-(2-methoxyethoxy)acetic acid (CAS number 16024-56-9) provided the title compound as a brown solid. LCMS (ESI+): RT=4.38 min, (M+H)=539.62; HRMS (ESI−): Predicted: 537.1402, found: 537.1392; ¹H NMR (300 MHz, DMSO-d₆) 5=7.43-7.33 (m, 2H), 7.13-7.01 (m, 2H), 6.96-6.84 (m, 2H), 4.43 (s, 4H), 3.69-3.48 (m, 8H), 1.79-1.66 (m, 6H).

### Example 397—Synthesis of 2-(2-{[2-(2-methoxyethoxy)ethyl]amino}acetyl)-5-{[2-(2-{[2-(2-methoxyethoxy)ethyl]amino}acetyl)-1,3-dioxo-2,3-dihydro-1H-inden-5-yl]sulfonyl}-2,3-dihydro-1H-indene-1,3-dione. FC-12967

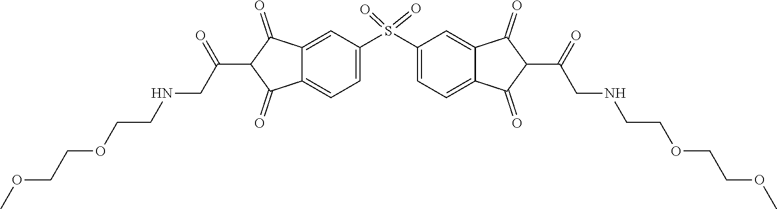

Using the procedure of Example 357, the tbutylcarboxyl moiety of tert-butyl N-{2-[5-({2-[2-(11,11-dimethyl-9-oxo-2,5,10-trioxa-8-azadodecan-8-yl)acetyl]-1,3-dioxo-2,3-dihydro-1H-inden-5-yl}sulfonyl)-1,3-dioxo-2,3-dihydro-1H-inden-2-yl]-2-oxoethyl}-N-[2-(2-methoxyethoxy)ethyl]carbamate (Example 392) was removed with trifluoroacetic acid. LCMS (ESI+): RT=2.90 min, (M+H)=673.89; HRMS (ESI−): Predicted: 671.1916, found: 671.1923; ¹H NMR (METHANOL-d₄) δ=8.23-8.12 (m, 2H), 8.07-8.01 (m, 2H), 7.75-7.63 (m, 2H), 4.33 (br s, 4H), 3.82-3.72 (m, 4H), 3.70-3.62 (m, 4H), 3.60-3.51 (m, 6H), 3.36 (s, 6H), 3.26 (br d, J=3.5 Hz, 2H).

### Example 398—Synthesis of [(2R,3S,4R,5R,6S)-3,4,6-tris(acetyloxy)-5-[5-(1,3-dioxo-2-{[(2S,3R,4R,5S,6R)-2,4,5-tris(acetyloxy)-6-[(acetyloxy)methyl]oxan-3-yl]carbamoyl}-2,3-dihydro-1H-indene-5-carbonyl)-1,3-dioxo-2,3-dihydro-1H-indene-2-amido]oxan-2-yl]methyl acetate. FC-13111

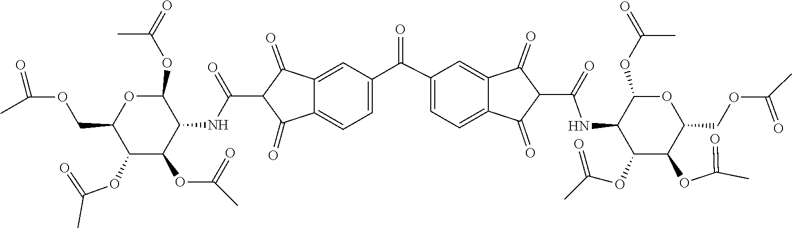 [SMILES: CC(=O)OC[C@H]1O[C@@H](OC(C)=O)[C@H](NC(=O)C2C(=O)c3ccc(C(=O)c4ccc5c(c4)C(=O)C(C(=O)N[C@H]4[C@H](OC(C)=O)O[C@H](COC(C)=O)[C@@H](OC(C)=O)[C@@H]4OC(C)=O)C5=O)cc3C2=O)[C@@H](OC(C)=O)[C@@H]1OC(C)=O]

1) To a vigorously-stirred mixture of β-D-glucopyranose 1,3,4,6-tetraacetate-2-amino-2-deoxy hydrochloride (CAS reg no: 10034-20-5, 768 mg, 2 mmol) in CH₂Cl₂ (19 ml) and saturated aqueous sodium bicarbonate (22 ml) at 0° C. under nitrogen was added triphosgene (890 mg, 3 mmol) in one portion. The reaction was stirred vigorously at this temperature for 30 min before the stopping and the layers were separated. The organic phase was washed (2×) with 1 N HCl (aq) (10 ml) and the combined organic layers were dried over sodium sulfate. The solvent was removed in vacuo and the crude acetic acid 4,5-diacetoxy-6-acetoxymethyl-3-isocyanato-tetrahydro-pyran-2-yl ester was used as-is in the following step.

2) Using the procedure of Example 335, reaction of 5,5′-carbonylbis(1H-indene-1,3(2H)-dione (Example 4) and acetic acid 4,5-diacetoxy-6-acetoxymethyl-3-isocyanato-tetrahydro-pyran-2-yl ester (Step 1 above) provided the title compound as a brown solid. LCMS (ESI+): RT=4.93 min, (M+Na)=1088.10; HRMS (ESI−): Predicted: 1063.2599, found: 1063.2529; ¹H NMR δ=8.50 (br d, J=10.0 Hz, 2H), 7.81 (br d, J=7.0 Hz, 2H), 7.59-7.42 (m, 2H), 6.04 (br d, J=8.8 Hz, 2H), 5.48 (br t, J=10.0 Hz, 2H), 4.89 (br t, J=9.4 Hz, 2H), 4.31-4.07 (m, 8H), 3.99 (br d, J=10.5 Hz, 2H), 2.02-1.93 (m, 18H), 1.90-1.82 (m, 3H).

### Example 399—Synthesis of [(2R,3S,4R,5R,6S)-3,4,6-tris(acetyloxy)-5-{5-[(1,3-dioxo-2-{[(2S,3R,4R,5S,6R)-2,4,5-tris(acetyloxy)-6-[(acetyloxy)methyl]oxan-3-yl]carbamoyl}-2,3-dihydro-1H-inden-5-yl)oxy]-1,3-dioxo-2,3-dihydro-1H-indene-2-amido}oxan-2-yl]methyl acetate. FC-13112

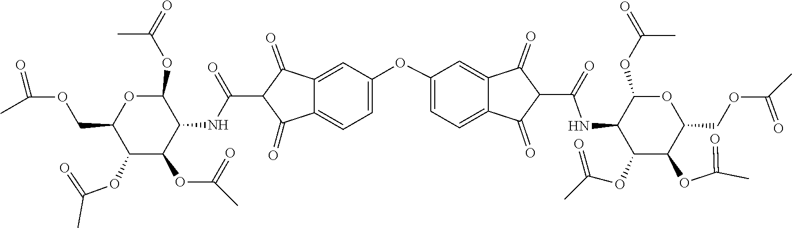 [SMILES: CC(=O)OC[C@H]1O[C@@H](OC(C)=O)[C@H](NC(=O)C2C(=O)c3ccc(Oc4ccc5c(c4)C(=O)C(C(=O)N[C@H]4[C@H](OC(C)=O)O[C@H](COC(C)=O)[C@@H](OC(C)=O)[C@@H]4OC(C)=O)C5=O)cc3C2=O)[C@@H](OC(C)=O)[C@@H]1OC(C)=O]

Using the procedure of Example 334, reaction of 5-(2,3-dihydro-1,3-dioxo-1H-inden-6-yloxy)-2,3-dihydro-1,3-dioxo-1H-indene (Example 7) and acetic acid 4,5-diacetoxy-6-acetoxymethyl-3-isocyanato-tetrahydro-pyran-2-yl ester (Example 398, Step 1) provided the title compound as a brown solid. LCMS (ESI+): RT=5.32 min, (M+Na)=1075.71; HRMS (ESI−): Predicted: 1051.2599, found: 1051.2609; ¹H NMR δ=8.52-8.17 (m, 2H), 7.44 (br s, 4H), 6.63-6.47 (m, 2H), 6.21-5.82 (m, 2H), 5.64-5.35 (m, 2H), 5.05-4.67 (m, 2H), 4.29-3.82 (m, 6H), 2.05-1.83 (m, 18H), 1.25-1.13 (m, 3H).

### Example 400—Synthesis of [(2R,3S,4R,5R,6S)-3,4,6-tris(acetyloxy)-5-{5-[(1,3-dioxo-2-{[(2S,3R,4R,5S,6R)-2,4,5-tris(acetyloxy)-6-[(acetyloxy)methyl]oxan-3-yl]carbamoyl}-2,3-dihydro-1H-inden-5-yl)sulfonyl]-1,3-dioxo-2,3-dihydro-1H-indene-2-amido}oxan-2-yl]methyl acetate. FC-13113

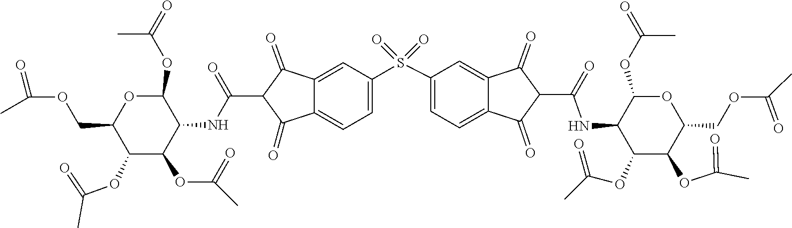 [SMILES: CC(=O)OC[C@H]1O[C@@H](OC(C)=O)[C@H](NC(=O)C2C(=O)c3ccc(S(=O)(=O)c4ccc5c(c4)C(=O)C(C(=O)N[C@H]4[C@H](OC(C)=O)O[C@H](COC(C)=O)[C@@H](OC(C)=O)[C@@H]4OC(C)=O)C5=O)cc3C2=O)[C@@H](OC(C)=O)[C@@H]1OC(C)=O]

Using the procedure of Example 336, reaction of 5,5′-sulfonylbis(1H-indene-1,3(2H)-dione) (Example 11) and acetic acid 4,5-diacetoxy-6-acetoxymethyl-3-isocyanato-tetrahydro-pyran-2-yl ester (Example 398, Step 1) provided the title compound as a brown solid. LCMS (ESI+): RT=4.58 min, (M+Na)=1123.87; HRMS (ESI−): Predicted: 1099.2269, found: 1099.2279.

### Example 401—Synthesis of (2-cyclopropanecarbonyl-5-(2-cyclopropanecarbonyl-1,3-dioxo-2,3-dihydro-1H-indene-5-carbonyl)-2,3-dihydro-1H-indene-1,3-dione. FC-13178

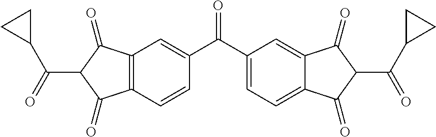 [SMILES: O=C(c1ccc2c(c1)C(=O)C(C(=O)C1CC1)C2=O)c1ccc2c(c1)C(=O)C(C(=O)C1CC1)C2=O]

Using the procedure of Example 347, reaction of 5,5′-carbonylbis(1H-indene-1,3(2H)-dione (Example 4) and cyclopropane carboxylic acid (CAS Number: 1759-53-1) provided the title compound as a brown solid. The compound was isolated as the bis sodium salt. LCMS (ESI+): RT=6.86 min, (M+H)⁺=455.32, (M+MeCN)+=496.29; HRMS (ESI−): calculated for C₂₇H₁₇O₇ m/z [M−H]⁻: 453.097976, observed. 453.098477; ¹H NMR δ: 7.84 (d, J=7.6 Hz, 2H), 7.63 (s, 2H), 7.52 (d, J=7.6 Hz, 2H), 3.31 (m, 2H, coincident with H₂O), 0.70-0.76 (m, 4H), 0.55-0.64 (m, 4H).

### Example 402—Synthesis of 2-cyclopropanecarbonyl-5-[(2-cyclopropanecarbonyl-1,3-dioxo-2,3-dihydro-1H-inden-5-yl)sulfonyl]-2,3-dihydro-1H-indene-1,3-dione. FC-13179

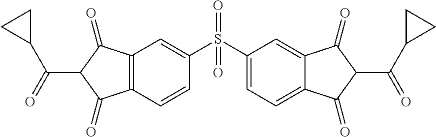 [SMILES: O=C1c2ccc(S(=O)(=O)c3ccc4c(c3)C(=O)C(C(=O)C3CC3)C4=O)cc2C(=O)C1C(=O)C1CC1]

Using the procedure of Example 348, reaction of 5,5′-sulfonylbis(1H-indene-1,3(2H)-dione) (Example 11) and cyclopropane carboxylic acid (CAS Number: 1759-53-1) provided the title compound as a brown solid. The compound was isolated as the bis sodium salt. LCMS (ESI+): RT=6.45 min, (M+H)⁺=491.30, (M+MeCN)+=532.41; HRMS (ESI−): calculated for C₂₆H₁₇O₈S m/z [M−H]⁻: 489.064962, observed. 489.063726; ¹H NMR δ: 8.08 (d, J=7.6 Hz, 2H), 7.74 (s, 2H), 7.55 (d, J=7.6 Hz, 2H), 3.31 (m, 2H, coincident with H₂O), 0.68-0.75 (m, 4H), 0.55-0.64 (m, 4H).

### Example 403—Synthesis of ethyl 3-(5-{[2-(3-ethoxy-3-oxopropanoyl)-1,3-dioxo-2,3-dihydro-1H-inden-5-yl]sulfonyl}-1,3-dioxo-2,3-dihydro-1H-inden-2-yl)-3-oxopropanoate. FC-13521

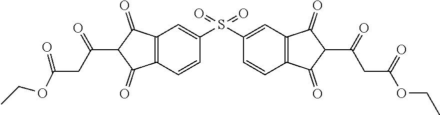 [SMILES: CCOC(=O)CC(=O)C1C(=O)c2ccc(S(=O)(=O)c3ccc4c(c3)C(=O)C(C(=O)CC(=O)OCC)C4=O)cc2C1=O]

Using the procedure of Example 348, reaction of 5,5′-sulfonylbis(1H-indene-1,3(2H)-dione) (Example 11) and monoethyl malonate (CAS number 1071-46-1) provided the title compound as a brown solid. The compound was isolated as the bis sodium salt. LCMS (ESI+): R_{f}=3.92, M+H=583.44; ¹H NMR δ: 8.10 (dd, J=7.62, 1.76 Hz, 1H), 7.77 (d, J=1.17 Hz, 1H), 7.57 (d, J=7.62 Hz, 1H), 4.00 (q, J=7.23 Hz, 2H), 3.74 (s, 2H), 1.13 (t, J=7.03 Hz, 3H).

### Example 404—Synthesis of 3-(5-{[2-(2-carboxyacetyl)-1,3-dioxo-2,3-dihydro-1H-inden-5-yl]sulfonyl}-1,3-dioxo-2,3-dihydro-1H-inden-2-yl)-3-oxopropanoic acid. FC-13522

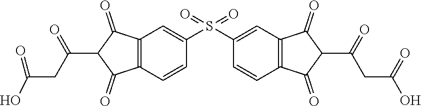 [SMILES: O=C(O)CC(=O)C1C(=O)c2ccc(S(=O)(=O)c3ccc4c(c3)C(=O)C(C(=O)CC(=O)O)C4=O)cc2C1=O]

To a 40 ml vial equipped with a stirbar was charged ethyl 3-(5-{[2-(3-ethoxy-3-oxopropanoyl)-1,3-dioxo-2,3-dihydro-1H-inden-5-yl]sulfonyl}-1,3-dioxo-2,3-dihydro-1H-inden-2-yl)-3-oxopropanoate (Example 403, 97.5 mg, 0.1556 mmol) and the solid was slurried in 5 ml of MeOH. To this was charged 5 ml of 1N NaOH and the resulting mixture was stirred overnight at ambient temperature. Once deemed complete by LCMS, 5 ml of 2N HCl was added and the resulting slurry was stirred at ambient temperature for NLT 5 minutes. The solid was then isolated by filtration, washed with 5 ml of 2N HCl and allowed to dry via suction. The solid was placed in the vacuum oven to dry overnight at 45° C. and full house vacuum providing the desired product. LCMS (ESI+): R_{f}=5.15; ¹H δ: 8.31 (dd, J=7.62 Hz, 1H), 8.10 (s, 1H), 7.82 (d, J=7.62 Hz, 1H), 2.44 (s overlaps with DMSO, 2H).

### Example A1—Inhibition of PF4 Tetramer Formation

Compounds of Examples 1-38, 65-301, 303, 305, 307, 309, 311, 313, 314, 316, and 318-404 were tested using SDS PAGE analysis of BS3 crosslinked PF4 for their ability to prevent PF4 tetramer formation. ULCs containing PF4 tetramers were distinguished from PF4 alone using dynamic light scattering (DLS). See, Suvarna et al., Throm. Res, 122:211-220 (2008), which is hereby incorporated by reference, at least for a detailed description of this technique.

cDNA encoding human PF4 was cloned into the plasmid μMT/BiPN5-His A (Invitrogen Corp., Carlsbad CA) for expression in the Drosophila Expression System (Invitrogen, Carlsbad, CA). Cloning was performed using Bg1 II and Age I cloning sites. A hexanucleotide encoding Bg1 II site was then eliminated by site-directed mutagenesis so that the expressed protein contained full-length wild type (wt) PF4 or PF4^{K50E} with an identical sequence as their counterparts expressed in E. coli. See, method described in Sachais et al (Sachais, B. S. et al. (2012). Blood 119(25): 5955-5961). PF4 expression was induced by adding copper sulfate (0.5 mM). The induced S₂ cells were incubated in serumfree Insect-Xpress™ media (Lonza, Walkersville, MD) for 3-5 days; supernatants were collected, sodium azide (0.02% final concentration) and EDTA (2.5 mM final concentration) were added, and the media were filtered through an Express® PLUS 0.22 μm filter (Millipore Corp., Billerica, MA).

Wild-type (WT) human PF4 in the pT7-7 vector (Novagen, Madison, WI) was expressed in the Escherichia coli strain BL21DE30 pLysS (Stratagene, La Jolla, CA), and purified and characterized as described by Rauova et al. (Rauova, L. et al. (2006). Blood 107(6): 2346-2353) and Park et al., (Blood, 75:1290-1295 (1990)), which are hereby incorporated by reference. Briefly, recombinant protein was isolated from the supernatant of the bacterial lysate by affinity chromatography using a HiTrap high-performance (HP) affinity column. WT PF4 was purified from the media on a heparin HiTrap® column on an ÄTKAPrime™ system (GE Healthcare) at 4° C. in Tris (10 mM), EDTA (1 mM), and pH 8 buffer. Media was loaded in buffer containing NaCl (0.5 M) and PF4 was eluted at 1.8 M NaCl using a linear gradient. Fractions containing purified PF4 as detected by silver staining of 12% NuPAGE Bis-Tris gels (Invitrogen) were pooled, concentrated and buffer exchanged into 50 mM HEPES, 0.5 M NaCl, pH—7.2 using an Amicon® Ultra centrifugal filter (3K NMWL, Millipore Corp). Protein was quantified using a BCA assay.

### Experiment 1

Compounds of Examples 1-38, 65-196, and 209-228 were then tested for their ability to inhibit PF4 tetramer formation using a cross-linking assay as described in Rauova cited above. Specifically, hPF4 (10 μg/ml) in phosphate-buffered saline (PBS) was incubated at 37° C. for 15 min in the absence of the antagonistic compounds (control sample) and presence of compounds. Cross-linking and SDS-PAGE analysis were then performed as described in “Analysis of PF4 multimerization” of Rauova. The relative amounts of PF4 in each of the monomer, dimer, trimer and tetramer form were calculated by analyzing the samples on a 12% SDS-polyacrylamide gel under reducing conditions. Bands were quantified using the Kodak ID Image Analysis system (Kodak, Rochester, NY). The SigmaMarker™ reagent served as the molecular weight standard. Data was compiled and bar graphed as % PF4 for the tetramer, trimer, dimer, and monomer as a function of concentration for the test and control samples. These data show that certain compounds were effective in inhibiting PF4 tetramer formation at tested concentrations.

### Experiment 2

This experiment was performed in a manner similar to experiment 1, but PF4 (10 μg/ml) in PBS was independently incubated with compounds of Examples 1-38, 65-196, and 209-228 for 60 min at room temperature, followed by the addition of cross-linking reagent bis(sulfosuccinimidyl)suberate (0.2 mM) for 30 min at room temperature. The reaction was stopped by adding NuPAGE® lithium dodecyl sulfate (LDS) sample buffer, and denatured by heating to 70° C. for 10 min according to the manufacturer's instructions. The analysis and compilation of the data also was performed as described above in Experiment 1. Table 1 shows the data for the PF4 tetramerization of these compounds.

### The IC₅₀ data for inhibition of PF4 tetramerization is shown in Table 1 below

TABLE 1

| Example No. | A¹ | B² | C³ | D⁴ | IC₅₀^{a} |
|---|---|---|---|---|---|
| Example 1 | | | | | 7.00 |
| Example 2 | | | | | 18.00 |
| Example 3 | | | 100 | 100 | 2.00 |
| Example 4 | | | 100 | 96 | 4.00 |
| Example 5 | | | | | 10.40 |
| Example 6 | | | | | 1.05 |
| Example 7 | | | | | 1.12 |
| Example 8 | | | | | 3.40 |
| Example 9 | | | | | 52.00 |
| Example 10 | | | | | 1.35 |
| Example 11 | | | | | 1.45 |
| Example 12 | | | | | 2.32 |
| Example 13 | 10 | 74 | 5 | 16 | |
| Example 14 | | | 30 | 96 | 1.00 |
| Example 15 | | | 30 | 93 | 3.00 |
| Example 16 | | | 30 | 78 | 20.00 |
| Example 17 | | | | | 1.08 |
| Example 18 | 10 | 75 | 5 | 65 | 1.81 |
| Example 19 | 10 | 56 | 5 | 29 | |
| Example 20 | 10 | 92 | 5 | 83 | 5.75 |
| Example 21 | 10 | 46 | 5 | 27 | |
| Example 22 | 10 | 76 | 5 | 66 | 1.87 |
| Example 23 | 10 | 92 | 5 | 84 | 1.75 |
| Example 24 | 10 | 89 | 5 | 83 | 5.11 |
| Example 25 | 10 | 70 | 5 | 63 | 6.24 |
| Example 26 | 10 | 66 | 5 | 59 | 3.61 |
| Example 27 | | | | | 10.75 |
| Example 28 | 10 | 65 | 5 | 82 | 0.64 |
| Example 29 | | | | | 3.02 |
| Example 30 | 10 | 58 | 5 | 12 | |
| Example 31 | | | | | 1.74 |
| Example 32 | 10 | 91 | 5 | 86 | 2.99 |
| Example 33 | 10 | 76 | 5 | 67 | 1.60 |
| Example 34 | 10 | 81 | 5 | 60 | 1.06 |
| Example 35 | 10 | 77 | 5 | 54 | 2.85 |
| Example 36 | 10 | 70 | 5 | 65 | 1.19 |
| Example 37 | | | | | ~1 |
| Example 38 | 10 | 88 | 5 | 74 | 1.07 |
| Example 65 | 10 | 71 | 5 | 50 | |
| Example 66 | 10 | 86 | 5 | 48 | |
| Example 67 | 10 | 20 | 5 | 0 | |
| Example 68 | 10 | 86 | 5 | 45 | 1.54 |
| Example 69 | 10 | 85 | 5 | 60 | 1.05 |
| Example 70 | 10 | ND | 5 | ND | |
| Example 71 | 10 | 85 | 5 | 2 | 1.00 |
| Example 72 | 10 | 52 | 5 | 43 | |
| Example 73 | 10 | 0 | 5 | 0 | |
| Example 74 | 10 | 46 | 5 | 19 | |
| Example 75 | 10 | 85 | 5 | 54 | |
| Example 76 | | | | | 2.1 |
| Example 77 | | | | | 2.3 |
| Example 78 | | | | | 1.4 |
| Example 79 | | | | | 1.0 |
| Example 80 | 10 | 90 | 5 | 90 | |
| Example 81 | | | | | 4.6 |
| Example 82 | | | | | 30 |
| Example 83 | | | | | 2.3 |
| Example 84 | | | | | 3.0 |
| Example 85 | | | | | 2.5 |
| Example 86 | 10 | 90 | 5 | 73 | |
| Example 87 | | | | | 2.5 |
| Example 88 | | | | | 21 |
| Example 89 | 10 | 66 | 5 | 38 | |
| Example 90 | | | | | 4.2 |
| Example 91 | 10 | 35 | 5 | 20 | |
| Example 92 | 10 | 65 | 5 | 60 | |
| Example 93 | 10 | 75 | 5 | 70 | |
| Example 94 | 10 | 62 | 5 | 33 | |
| Example 95 | 10 | 69 | 5 | 33 | |
| Example 96 | 10 | 70 | 5 | 36 | |
| Example 97 | 10 | 100 | 5 | 100 | 8.9 |
| Example 98 | 10 | 100 | 5 | 100 | |
| Example 99 | 10 | 100 | 5 | 44 | 13 |
| Example 100 | | | | | ~13 |
| Example 101 | 10 | 71 | 5 | 80 | |
| Example 102 | 10 | 100 | 5 | 78 | 2.9 |
| Example 103 | 10 | 77 | 5 | 66 | 0.5 |
| Example 104 | | | | | 1.4 |
| Example 105 | | | | | 2.8 |
| Example 106 | | | | | ~4 |
| Example 107 | | | | | 2.9 |
| Example 108 | | | | | 5.4 |
| Example 109 | 10 | 59 | 5 | 68 | |
| Example 110 | 10 | 87 | 5 | 84 | ~4 |
| Example 111 | 10 | 100 | 5 | 100 | 0.5 |
| Example 112 | 10 | 89 | 5 | 84 | 1.2 |
| Example 113 | 10 | 86 | 5 | 68 | 5.4 |
| Example 114 | 10 | 91 | 5 | 76 | 2.5 |
| Example 115 | | | | | 3.3 |
| Example 116 | 10 | 21 | 5 | 13 | |
| Example 117 | | | | | IN |
| Example 118 | 10 | 94 | 5 | 75 | 3.4 |
| Example 119 | 10 | 90 | 5 | 83 | 4 |
| Example 120A | 10 | 80 | 5 | 51 | 5.1 |
| Example 120B | 10 | 86 | 5 | 75 | |
| Example 121 | 10 | 80 | 5 | 82 | 3.5 |
| Example 122 | | | | | ~3 |
| Example 123 | 10 | 89 | 5 | 88 | 1.6 |
| Example 124 | | | | | 6.3 |
| Example 125 | 10 | 93 | 5 | 87 | 1.2 |
| Example 126 | 10 | 89 | 5 | 88 | 3.1 |
| Example 127 | | | | | 1.0 |
| Example 128 | 10 | 87 | 5 | 51 | 7.8 |
| Example 129 | | | | | ~2 |
| Example 130 | | | | | 1.4 |
| Example 131 | 10 | 88 | 5 | 83 | 4.1 |
| Example 132 | 10 | 88 | 5 | 79 | 3.5 |
| Example 133 | 10 | 88 | 5 | 66 | ~6 |
| Example 134 | | | | | 1.1 |
| Example 135 | 10 | 88 | 5 | 79 | 3.8 |
| Example 136 | 10 | 86 | 5 | 75 | 18 |
| Example 137 | 10 | 85 | 5 | 80 | 6 |
| Example 138 | | | | | 4.1 |
| Example 139 | 10 | 97 | 5 | 87 | 7 |
| Example 140 | 10 | 93 | 5 | 92 | 4.7 |
| Example 141 | 10 | 68 | 5 | 38 | |
| Example 142 | 10 | 94 | 5 | 95 | 18 |
| Example 143 | | | | | 7.0 |
| Example 144 | | | | | 4.7 |
| Example 145 | 10 | 55 | 5 | 45 | |
| Example 146 | 10 | 40 | 5 | 44 | |
| Example 147 | 10 | 59 | 5 | 55 | |
| Example 148 | 10 | 90 | 5 | 59 | |
| Example 149 | 10 | 53 | 5 | 64 | |
| Example 150 | 10 | 75 | 5 | 50 | ~22 |
| Example 151 | 10 | 100 | 5 | 89 | ~6 |
| Example 152 | 10 | 100 | 5 | 77 | ~22 |
| Example 153 | 10 | 100 | 5 | 100 | 2.4 |
| Example 154 | 10 | 100 | 5 | 100 | 1.2 |
| Example 155 | 10 | 100 | 5 | 95 | 2.1 |
| Example 156 | 10 | 100 | 5 | 94 | 6.3 |
| Example 157 | 10 | 100 | 5 | 100 | 1.2 |
| Example 158 | 10 | 100 | 5 | 100 | 1.4 |
| Example 159 | 10 | 90 | 5 | 85 | 3.8 |
| Example 160 | 10 | 88 | 5 | 75 | 5.1 |
| Example 161 | 10 | 93 | 5 | 87 | 1.0 |
| Example 162 | 10 | 94 | 5 | 86 | 2.6 |
| Example 163 | 10 | 93 | 5 | 87 | 13 |
| Example 164 | 10 | 82 | 5 | 70 | 5.0 |
| Example 165 | 10 | 75 | 5 | 75 | 3.3 |
| Example 166 | 10 | 84 | 5 | 59 | 3.4 |
| Example 167 | 10 | 36 | 5 | 18 | |
| Example 168 | 10 | 89 | 5 | 68 | 4.3 |
| Example 169 | 10 | 86 | 5 | 70 | 3.0 |
| Example 170 | 10 | 85 | 5 | 76 | 3.4 |
| Example 171 | 10 | 75 | 5 | 62 | |
| Example 172 | 10 | 93 | 5 | 81 | 1.6 |
| Example 173 | 10 | 97 | 5 | 87 | 2.2 |
| Example 174 | 10 | 94 | 5 | 84 | 11 |
| Example 175 | 10 | 97 | 5 | 90 | 1.1 |
| Example 176 | 10 | 77 | 5 | 21 | |
| Example 177 | 10 | 17 | 5 | 0 | |
| Example 178 | 10 | 75 | 5 | 22 | |
| Example 179 | 10 | 92 | 5 | 81 | ~6 |
| Example 180 | 10 | 90 | 5 | 73 | ~6 |
| Example 181 | 10 | 96 | 5 | 86 | 1.6 |
| Example 182 | 10 | 88 | 5 | 74 | 5.7 |
| Example 183 | 10 | 100 | 5 | 87 | 4.3 |
| Example 184 | 10 | 95 | 5 | 87 | 2.0 |
| Example 185 | 10 | 94 | 5 | 89 | 4.4 |
| Example 186 | 10 | 95 | 5 | 95 | 2.6 |
| Example 187 | 10 | 90 | 5 | 88 | 2.6 |
| Example 188 | 10 | 94 | 5 | 88 | 2.30 |
| Example 189 | 10 | 71 | 5 | 62 | |
| Example 190 | 10 | 86 | 5 | 79 | |
| Example 191 | 10 | 79 | 5 | 74 | ~3 |
| Example 192 | | | | | IN |
| Example 193 | 10 | 40 | 5 | 37 | |
| Example 194 | | | | | ~1.5 |
| Example 195 | | | | | 4.0 |
| Example 196 | 10 | 96 | 5 | 88 | 5.3 |
| Example 197 | 10 | 62 | 5 | 45 | |
| Example 198 | 10 | 70 | 5 | 59 | |
| Example 199 | 10 | 78 | 5 | 52 | |
| Example 200 | 10 | 63 | 5 | 62 | |
| Example 201 | 10 | 93 | 5 | 74 | >30 |
| Example 202 | 10 | 0 | 5 | 0 | |
| Example 203 | 10 | 51 | 5 | 7 | |
| Example 204 | 10 | 47 | 5 | 0 | |
| Example 205 | 10 | 27 | 5 | 15 | |
| Example 206 | 10 | 36 | 5 | 36 | |
| Example 207 | 10 | 75 | 5 | 63 | 4.7 |
| Example 208 | 10 | 61 | 5 | 43 | |
| Example 209 | 10 | 96 | 5 | 75 | 4.2 |
| Example 210 | 10 | 94 | 5 | 62 | ~5 |
| Example 211 | 10 | 95 | 5 | 76 | 3.3 |
| Example 212 | 10 | 88 | 5 | 91 | 5.5 |
| Example 213 | 10 | 89 | 5 | 87 | 4.8 |
| Example 214 | 10 | | 5 | | 4.0 |
| Example 215 | 10 | | 5 | | 2.4 |
| Example 216 | 10 | 91 | 5 | 82 | 2.5 |
| Example 217 | 10 | 95 | 5 | 91 | 1.2 |
| Example 218 | 10 | 92 | 5 | 67 | 4.0 |
| Example 219 | 10 | 91 | 5 | 68 | 5.0 |
| Example 220 | 10 | 92 | 5 | 90 | 2.5 |
| Example 221 | 10 | 97 | 5 | 92 | 2.3 |
| Example 222 | 10 | 97 | 5 | 94 | 1.4 |
| Example 223 | 10 | 100 | 5 | 97 | 1.4 |
| Example 224 | 10 | 95 | 5 | 100 | 2.2 |
| Example 225 | 10 | 90 | 5 | 87 | 3.2 |
| Example 226 | 10 | 92 | 5 | 77 | 4.0 |
| Example 227 | | | | | 1.8 |
| Example 228 | | | | | 2.8 |
| Example 229 | 3 | 53 | 1 | 43 | |
| Example 230 | 3 | 24 | 1 | 1 | |
| Example 231 | 3 | 65 | 1 | 69 | 1.71 |
| Example 232 | 3 | 67 | 1 | 10 | |
| Example 233 | 3 | 3 | 1 | 1 | |
| Example 234 | 3 | 72 | 1 | 21 | 1.51 |
| Example 235 | 3 | 29 | 1 | 11 | |
| Example 236 | 3 | 25 | 1 | 20 | |
| Example 237 | 3 | 37 | 1 | 30 | |
| Example 238 | 3 | 100 | 1 | 7 | |
| Example 239 | 3 | 100 | 1 | | |
| Example 240 | 3 | 32 | 1 | 14 | |
| Example 241 | 3 | 29 | 1 | 14 | |
| Example 242 | 3 | 31 | 1 | 13 | |
| Example 243 | 3 | 21 | 1 | 19 | |
| Example 244 | 3 | 43 | 1 | 21 | |
| Example 245 | 3 | 47 | 1 | 27 | |
| Example 246 | 3 | 30 | 1 | 12 | 3 |
| Example 247 | 3 | 27 | 1 | 19 | 3 |
| Example 248 | 3 | 56 | 1 | 26 | 3 |
| Example 249 | 3 | 36 | 1 | 19 | 3 |
| Example 250 | 3 | 42 | 1 | 26 | 3 |
| Example 251 | 3 | 45 | 1 | 32 | 3 |
| Example 252 | 3 | 37 | 1 | 14 | 3 |
| Example 253 | 3 | 20 | 1 | 20 | |
| Example 254 | 3 | 51 | 1 | 28 | |
| Example 255 | 3 | 42 | 1 | 18 | |
| Example 255 | 3 | 51 | 1 | 9 | |
| Example 256 | 3 | 32 | 1 | 13 | |
| Example 257 | 3 | 37 | 1 | 7 | |
| Example 258 | 3 | 33 | 1 | 18 | |
| Example 259 | 3 | 55 | 1 | 18 | |
| Example 260 | 3 | 46 | 1 | 11 | |
| Example 261 | 3 | 77 | 1 | 67 | 1.32 |
| Example 262 | 3 | 67 | 1 | 49 | 1.33 |
| Example 263 | 3 | 69 | 1 | 72 | 2.07 |
| Example 264 | 3 | 75 | 1 | 72 | 9.83 |
| Example 265 | 3 | 34 | 1 | 18 | |
| Example 266 | 3 | 25 | 1 | | |
| Example 267 | 10 | 100 | | | |
| Example 268 | 3 | 25 | 1 | 7 | |
| Example 269 | 3 | 50 | 1 | 14 | |
| Example 270 | 3 | 24 | 1 | 10 | |
| Example 271 | 3 | 38 | 1 | 21 | |
| Example 272 | 3 | 56 | 1 | 26 | |
| Example 273 | 3 | 20 | 1 | 20 | |
| Example 274 | 3 | 60 | 1 | 21 | |
| Example 275 | 3 | 31 | 1 | 5 | 3 |
| Example 276 | 3 | 7 | 1 | 12 | 3 |
| Example 277 | 3 | 41 | 1 | 38 | 3 |
| Example 278 | 3 | 18 | 1 | 1 | 3 |
| Example 279 | 3 | 15 | 1 | 33 | 3 |
| Example 280 | 3 | 43 | 1 | 23 | 3 |
| Example 281 | 10 | 58 | | | 10 |
| Example 282 | 3 | 45 | 1 | 29 | 3 |
| Example 283 | 10 | 86 | | | 10 |
| Example 284 | 10 | 81 | | | 10 |
| Example 285 | 3 | 49 | 1 | 33 | 3 |
| Example 286 | 3 | 47 | 1 | 37 | 3 |
| Example 287 | 3 | 31 | 1 | 5 | 3 |
| Example 288 | 3 | 15 | 1 | 10 | 3 |
| Example 289 | 3 | 52 | 1 | 27 | |
| Example 290 | 3 | 51 | 1 | 39 | |
| Example 291 | 3 | 100 | 1 | 69 | 1.29 |
| Example 292 | 3 | 100 | 1 | 47 | 1.26 |
| Example 293 | 3 | 43 | 1 | 28 | |
| Example 294 | 3 | 88 | 1 | 45 | 1.97 |
| Example 295 | 3 | 100 | 1 | 29 | 1.10 |
| Example 296 | 3 | 36 | 1 | 22 | |
| Example 297 | 3 | 100 | 1 | 38 | 1.74 |
| Example 298 | 3 | 60 | 1 | 32 | |
| Example 299 | 3 | 56 | 1 | 32 | |
| Example 300 | 3 | 32 | 1 | 22 | |
| Example 301 | 3 | 30 | 1 | 23 | |
| Example 303 | 10 | 60 | | | |
| Example 305 | 3 | 50 | 1 | 13 | |
| Example 307 | 3 | 47 | 1 | 15 | |
| Example 309 | 3 | 32 | 1 | 20 | |
| Example 311 | 10 | 73 | | | |
| Example 313 | 3 | 67 | 1 | 31 | |
| Example 314 | 3 | 28 | 1 | 9 | |
| Example 316 | 3 | 50 | 1 | 25 | |
| Example 318 | 3 | 36 | 1 | 15 | |
| Example 319 | 3 | 22 | 1 | 13 | |
| Example 320 | 3 | 26 | 1 | 12 | |
| Example 321 | 3 | 23 | 1 | 17 | |
| Example 322 | 10 | 40 | | | |
| Example 323 | 3 | 18 | 1 | 7 | |
| Example 324 | 10 | 35 | | | |
| Example 325 | 10 | 34 | | | |
| Example 326 | 3 | 26 | 1 | 13 | |
| Example 327 | 3 | 24 | 1 | 13 | |
| Example 328 | 3 | 26 | 1 | 16 | |
| Example 329 | 3 | 50 | 1 | 22 | |
| Example 330 | 3 | 22 | 1 | 6 | |
| Example 331 | 3 | 66 | 1 | 37 | |
| Example 332 | 3 | 13 | 1 | 5 | |
| Example 333 | 3 | 21 | 1 | 15 | |
| Example 334 | 3 | 40 | 1 | 27 | |
| Example 335 | 3 | 39 | 1 | 23 | |
| Example 336 | 3 | 35 | 1 | 25 | |
| Example 337 | 3 | | 1 | | |
| Example 336 | 3 | | 1 | | |
| Example 337 | 3 | | 1 | | |
| Example 338 | 3 | | 1 | | |
| Example 339 | 3 | | 1 | | |
| Example 340 | 3 | | 1 | | |
| Example 341 | 3 | | 1 | | |
| Example 342 | 3 | | 1 | | |
| Example 343 | 3 | | 1 | | |
| Example 344 | 3 | | 1 | | |
| Example 345 | 3 | | 1 | | |
| Example 346 | 3 | | 1 | | |
| Example 347 | 3 | | 1 | | |
| Example 349 | 3 | 27 | 1 | 2 | |
| Example 350 | 3 | 39 | 1 | 18 | |
| Example 351 | 3 | 21 | 1 | 12 | |
| Example 352 | 3 | 78 | 1 | 35 | |
| Example 353 | 3 | 37 | 1 | 22 | |
| Example 354 | 3 | 32 | 1 | 26 | |
| Example 355 | 3 | 61 | 1 | 23 | |
| Example 356 | 3 | 55 | 1 | 18 | |
| Example 357 | 3 | 74 | 1 | 39 | 1.35 |
| Example 358 | 3 | 46 | 1 | 24 | |
| Example 359 | 3 | 45 | 1 | 34 | |
| Example 360 | 3 | 59 | 1 | 23 | |
| Example 361 | 3 | 34 | 1 | 9 | |
| Example 362 | 3 | 53 | 1 | 33 | |
| Example 363 | 3 | 63 | 1 | 29 | 3.96 |
| Example 364 | 3 | 74 | 1 | 13 | 1.35 |
| Example 365 | 3 | 63 | 1 | 27 | 3.39 |
| Example 366 | 3 | 60 | 1 | 41 | 0.55 |
| Example 367 | 3 | 77 | 1 | 30 | |
| Example 368 | 3 | 45 | 1 | 1 | |
| Example 369 | 3 | 21 | 1 | 1 | |
| Example 370 | 3 | 6 | 1 | 3 | |
| Example 371 | 3 | 11 | 1 | 7 | |
| Example 372 | 3 | 30 | 1 | | |
| Example 373 | 3 | 3 | 1 | 5 | |
| Example 374 | 3 | 49 | 1 | 22 | |
| Example 375 | 3 | 32 | 1 | 14 | |
| Example 376 | 3 | 62 | 1 | 22 | |
| Example 377 | 3 | 41 | 1 | 26 | |
| Example 378 | 3 | 69 | 1 | 39 | |
| Example 379 | 3 | 68 | 1 | 43 | 0.66 |
| Example 380 | 3 | 30 | 1 | 17 | |
| Example 381 | 3 | 19 | 1 | 7 | |
| Example 382 | 3 | 70 | 1 | 20 | 0.29 |
| Example 383 | 3 | 45 | 1 | 20 | |
| Example 384 | 3 | 9 | 1 | 4 | |
| Example 385 | 3 | 59 | 1 | 29 | |
| Example 386 | 3 | 34 | 1 | 13 | |
| Example 387 | 3 | 52 | 1 | 16 | |
| Example 388 | 3 | 31 | 1 | 23 | |
| Example 389 | 3 | 80 | 1 | 31 | |
| Example 390 | 3 | 31 | 1 | 27 | |
| Example 391 | 3 | 6 | 1 | 1 | |
| Example 392 | 3 | 1 | 1 | 1 | |
| Example 393 | 3 | 4 | 1 | 1 | |
| Example 394 | 3 | 5 | 1 | 2 | |
| Example 395 | 3 | 13 | 1 | 2 | |
| Example 396 | 3 | 17 | 1 | 14 | |
| Example 397 | 3 | 9 | 1 | 2 | |
| Example 398 | 3 | 23 | 1 | 6 | |
| Example 399 | 3 | 28 | 1 | 21 | |
| Example 400 | 3 | 21 | 1 | 17 | |
| Example 401 | 3 | 23 | 1 | 22 | |
| Example 402 | 3 | 28 | 1 | 24 | |
| Example 403 | 3 | 23 | 1 | 22 | |
| Example 404 | 3 | 28 | 1 | 24 | |

¹Single concentration of inhibitor used in the PF4 tetramer formation inhibition assay (μM)
²% inhibition of PF4 tetramer formation at that single concentration of inhibitor (column A)
³Single concentration of inhibitor used in the PF4 tetramer formation inhibition assay (μM)
⁴% inhibition of PF4 tetramer formation at that single concentration of inhibitor (column C)
^{a}IC₅₀ of PF4 tetramer formation inhibition (μM)
IN = not soluble in test media

In general, the active compounds are acidic and have low log 0 values. The clog 0 (pH 7.4) calculated values range from 1.7 to −13 and therefore should generally have good aqueous solubility. Since most compounds are bis-acids, with pKa values ranging from 6.5 to −1.7, most should have poor cell permeability. These characteristics are beneficial for IV administration and also for engagement of the molecular target PF4 which is a blood protein. For example, the compound in Example 1 has an experimentally determined log D value of 0.53 and a pKa of 1.99. It has experimentally determined kinetic solubility of >200 μM at pH 7.4.

### Example A2—Inhibition of Formation of Ultra Large Complexes (ULC) of PF4 Tetramers And Heparin

Since ULCs are formed as part of a dynamic equilibrium between heparin and PF4, dynamic light scattering (DLS) was performed to measure the ability of the exemplified compounds to disrupt preformed ULCs. Samples were analyzed and included PF4 incubated in the absence or presence of unfractionated heparin (UFH) for 20 min at room temperature at PF4 to heparin ratios (PHR) of 1:0 (no heparin), 2:1, and 0.64:1. The samples were then analyzed using photon correlation spectroscopy on a DynaPro® DLS instrument and Dynamics® software (V6.7.6; Microsoft) to obtain correlation function. The results of the DLS analysis indicate the percentage of ULCs and small PF4 particles. Data for the small particles (a population of particles with a mean hydrodynamic diameter of about 1 nm) and data for the large particles (a population of particles with a mean hydrodynamic diameter of about 300 nm) is assessed. Data are expressed as the percent of total intensity from each measurement, the mean of 10 measurements, and representative of two such experiments. Larger amounts of heparin result in the disruption of larger particles, i.e., ULCs, demonstrated by the presence of virtually all of the PF4 as small particles (data not shown).

Since the PF4 antagonists discussed herein are capable of disrupting preformed ULCs, the following experiment was performed to determine if the exemplified compounds inhibit formation of ULCs.

ELISA measurement and gel filtration measurement of ULC inhibition were performed as described in International Patent Publication No. WO2013/142328, which is incorporated herein by reference. Similarly, assays in which heparin concentrations are varied to determine the effect on ULC inhibition were performed as described in that publication.

Certain compounds were evaluated for their ability to inhibit formation of ultra large complexes (ULC) between PF4 tetramers and heparin. The results are illustrated in Table 2 below.

TABLE 2
ULC Inhibition and Cytotoxicity

| | ULC Inhibition | Cytotox HepG2 | Cytotox NIH3T3 |
|---|---|---|---|
| Example No. | IC₅₀ (μM)^{a} | EC₅₀ (μg/ml) | EC₅₀ (μg/ml)^{b} |
| Example 1 | 8 | >1000 | >1000 |
| Example 3 | 2 | >500 | >500 |
| Example 4 | 3 | >250 | >250 |
| Example 5 | 10 | >3353 | >3353 |
| Example 15 | 11 | >1000 | >1000 |

^{a}IC₅₀ values are +/−20%.
^{b}Cells are exposed to compounds for 1 hour in the absence of serum. Compounds are removed and metabolically active cells are measured using MTS reduction, N = 3.
None of the tested compounds were cytotoxic. This observation is in line with the finding of the previous example that the exemplified compounds apparently lack the ability to permeate cell membranes.

### Example A3—Modulation of Antibody Binding to ULC

The effect of the exemplified compounds on antibody binding to intact ULCs was evaluated. ULC were formed as described above, with the exception that the PF4 was incubated with heparin for 30 min. Certain compounds (1 mM) were then added to wells pre-coated with KKO (monoclonal hit like antibody). These solutions were then incubated in the KKO wells overnight at 37° C. Antibody binding was detected by adding HRP (horseradish peroxicase)-conjugated sheep polyclonal anti-human PF4 antibody and developed with TMB substrate. After stopping the enzymatic reaction with 1M H₃PO₄, absorbance was measured at 450 nm in a Packard SpectraCount™ plate reader.

### Example A4—Inhibition of Platelet Activation and Serotonin Release Assay

Since one of the therapeutic goals of using ULC antagonists in patients with HITT is to prevent activation, compounds were evaluated for the ability to inhibit platelet activation. Data were generated using a serotonin release assay as described in Hirschman, Br. J. Haematol., 24(6):793-802 (1973), and Rauova and Cines, N. Engl. J. Med., 303:788-795 (1980), which are hereby incorporated by reference. See also WO2013/142328.

In this assay, platelet rich plasma (PRP) from healthy donors was incubated with 0.5 μL ¹⁴C-5-hydroxytryptamine creatinine sulfate (GE Life Sciences, Piscataway, NJ) per milliliter of PRP for 20 min at 37° C. to produce ¹⁴C-labeled platelets. Serotonin uptake is inhibited by adding 1 mmol/ml imipramine (Sigma-Aldrich, St. Louis, MO) to the PRP. The radiolabeled platelets are mixed with KKO (170 μg/ml) or with known platelet-activating HIT plasma in the absence (buffer control) or presence of a selected PF4 antagonist compound of Table 1 (2.5-3.0 mM).

Negative and positive controls contain sera from patients previously known to have negative or positive serotonin release, respectively. The assay was performed in the presence of heparin (1.0 U/ml), i.e., heparin added to the positive control serum, and in the absence of heparin (background). The percent release of serotonin is calculated for all conditions. Negative controls without antibody were studied in parallel. ¹⁴C-5-hydroxytryptamine released from platelets was measured by scintillation counter. Data are expressed as % maximal release of radioactivity with release by the positive control plasma and 1.0 U/ml heparin defined as 100%. Compounds that inhibit platelet activation by KKO are measured by the serotonin release assay. (Rauova, L. (2005). Blood 105(1): 131-138).

### Example A5—ADMET (Absorption, Distribution, Metabolism, Excretion and Toxicity) Properties

Certain exemplified compounds were examined for ADMET (absorption, distribution, metabolism, excretion and toxicity) profiling using conventional assays with the results shown in the Tables 3A-3C below.

TABLE 3A
ADMET Human and Mouse Microsome stability data

| | Microsome stability | | | |
|---|---|---|---|---|
| | Mouse | | Human | |
| | Compound conc = 0.5 μM, n = 1)) | | (Compound conc = 0.5 μM, n = 1) | |
| | | Intrinsic Clearance | | Intrinsic Clearance |
| | Half life | Clᵢₙₜ (ml/min/mg | Half life | Clᵢₙₜ (ml/min/mg |
| Compound No. | t_{1/2} (min) | protein) | t_{1/2} (min) | protein) |
| Example 1 | >145 | <38 | 86.6 | 15.8 |
| Example 3 | >145 | <38 | >145 | <9.5 |
| Example 4 | 1.7 | 3296 | 4.7 | 293.0 |
| Example 20 | >145 | <38 | >145 | <9.5 |
| Example 15 | 93.8 | 0.709 | 116.3 | 0.473 |
| Example 11 | 105.3 | 0.632 | >180 | <0.3 |
| Example 34 | 86.5 | 0.769 | >180 | <0.3 |
| Example 22 | 47.9 | 1.39 | 59.7 | 0.921 |
| Example 28 | 52.2 | 1.275 | >180 | <0.3 |
| Example 32 | >180 | <0.3 | 54 | 1.019 |
| Midazolam | 5.3 | 12.506 | 4 | 13.77 |

TABLE 3B
ADMET P450 Inhibition

| | P450 Inhibition IC₅₀ (μM) | | | | |
|---|---|---|---|---|---|
| | | | | | 3A4 |
| Compound No. | 1A2 | 2C9 | 2C19 | 2D6 | (midazolam) |
| Example 1 | 46.7 | 31.9 | >100 | >100 | >100 |
| Example 3 | >100 | 16.7 | >100 | >100 | >100 |

TABLE 3C
ADMET Kinetic solubility and human Ether-à-go-go-
Related Gene (hERG) and plasma protein binding (PPB, mouse).

| | PPB mouse | Kinetic Solubility | hERG IC₅₀ |
|---|---|---|---|
| Compound No. | (% bound) | pH = 7.4 (μM) | (μM) |
| Example 1 | NT | >200 | >30 |
| Example 3 | 95.7 | 88.2 | >30 |

As shown by Tables 3A-3C, for representative compounds described herein, no serious ADMET issues have arisen and the compounds meet drug property criteria for intravenous administration.

Compounds of Example 1, Example 3 and Example 20 showed excellent stability and low intrinsic clearance in mouse and human microsome assays. In addition, Example 3 showed good aqueous solubility and high protein binding. Examples 1 and 3 were evaluated as inhibitors of the five P450s that are the source of most DDI issues. Both compounds showed good profiles with no inhibition of 2C19,2D6 or 3A4 midazolam and only moderate inhibition of 2C9. Example 1 had moderate inhibition of 1A2 as well. These two compounds were also evaluated in a hERG patch clamp assay and no significant inhibition was observed at 30 μM concentration. The tested compounds meet drug property criteria for IV administration.

### Example A6—Pharmacokinetic Parameters

Table 4 shows the results in pharmacokinetic (PK) parameters for compound of Example 1 after CD-1 mice were administered an intravenous bolus dose of the compound at 1 mg/kg (0.500 mg/ml in 5% DMSO/95% PBS (pH8)) for an average of 3 mice. PK parameters were measured and reported in Table 4 as follows: C₀ represents concentration in ng/ml at t=0 min; AUC₀₋ₗₐₛₜ represents area under the curve for t=0 min to last time point; AUC_{0-∞} represents area under the curve for t=0 min to infinity.

TABLE 4
PK Parameters

| | | | | Volume of | |
|---|---|---|---|---|---|
| | | AUC₀₋ₗₐₛₜ | AUC_{0-∞} | distribution | Clearance |
| t_{1/2} (hr) | C₀ (ng/ml) | (ng*h/ml) | (ng*h/ml) | (Vdₛₛ₎ in L/kg | (ng/ml) |
| 4.59 ± 0.584 | 15078 ± 2071 | 9188 ± 704 | 9348 ± 778 | 0.448 ± 0.0121 | 1.79 ± 0.147 |

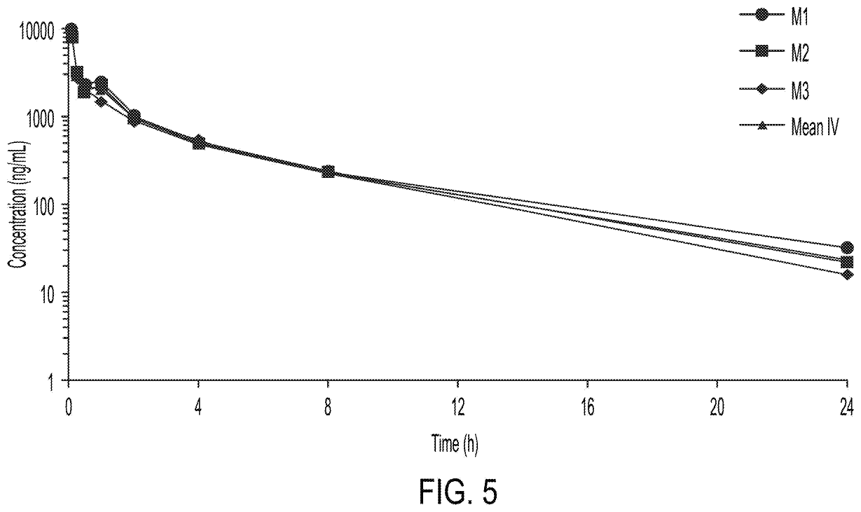

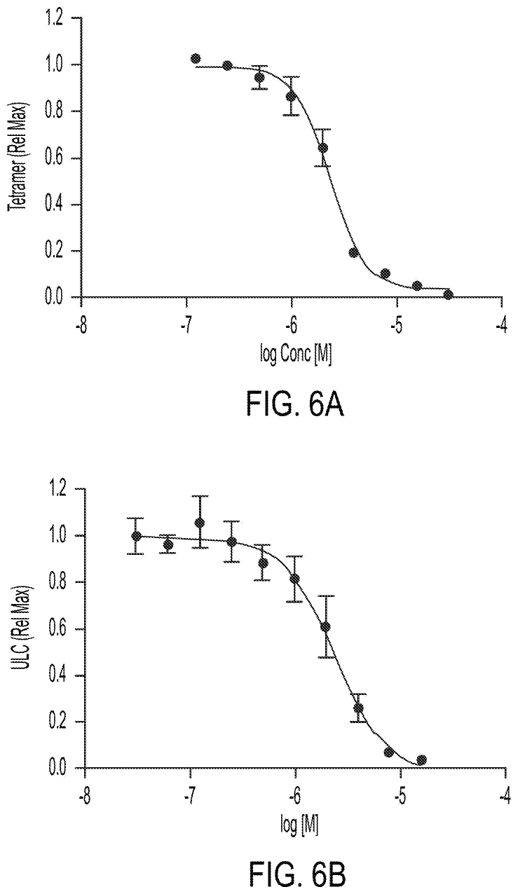

Compound of Example 1 showed a long half-life, low clearance and volume of distribution (see Table 4), all good parameters for an IV drug interacting with an extracellular blood protein. The concentration of Example 1 was sustained above 1 μM for greater than 4 hours at 1 mg/kg (see FIG. 5).

### Example A7—In Vivo Activity

Heparin-PF4-KKO mice that were induced to thrombocytopenia were administered the compound of Example 1. The dosing regimen was guided by the PK results in Table 4 (see Example A6). At T₀ₕᵣ, 5 mg/kg of Compound 1 was given by retro-orbital injection. 1000 units/kg heparin was injected subcutaneously at T₃ₕᵣ. At T₄ₕᵣ, 20 mg/kg of KKO antibody was administered by retro-orbital injection. Also at T₄ₕᵣ, an additional 5 mg/kg of the compound was injected intraperitoneally. Platelet count was measured at T₈ₕᵣ.

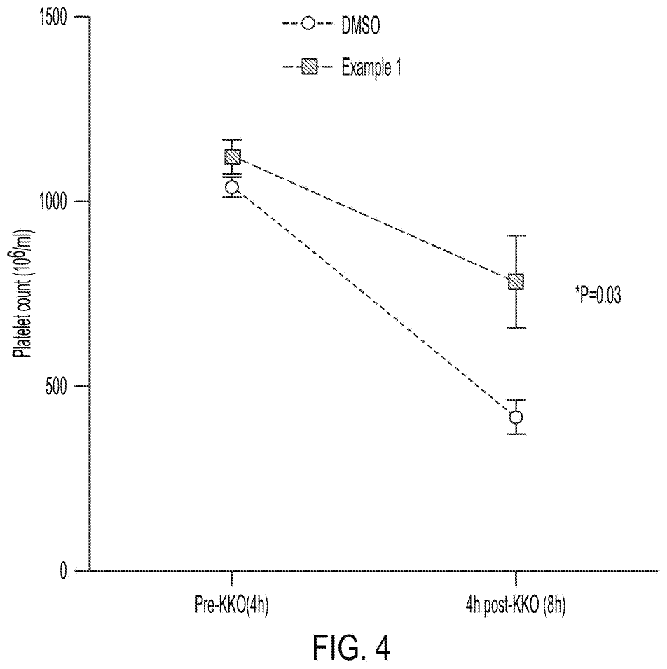

Results are shown in the graph of FIG. 4. The mice tolerated the 5 mg/kg IV and IP doses of the compound well. Three of the four mice tested with the compound of Example 1 showed almost complete protection, with platelet counts 4 hr after KKO administration at >80% of baseline, while the remaining mouse showed platelet counts at 4 hr only a little higher than DMSO controls. The results show that the compound of Example 1 reduced heparin-PF4-KKO induced thrombocytopenia in vivo and ameliorated the low platelet count seen with vehicle control.

### Example A8—P-Selectin Inhibition Assay

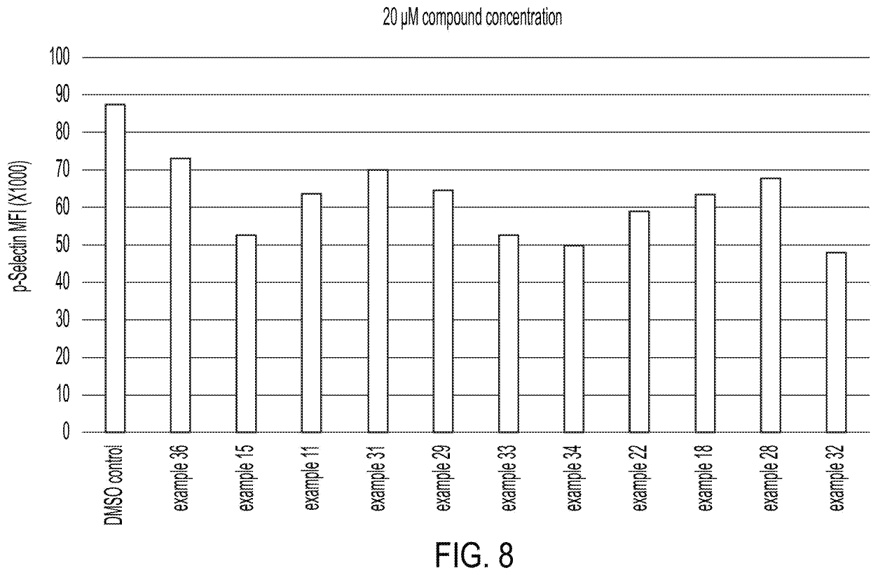

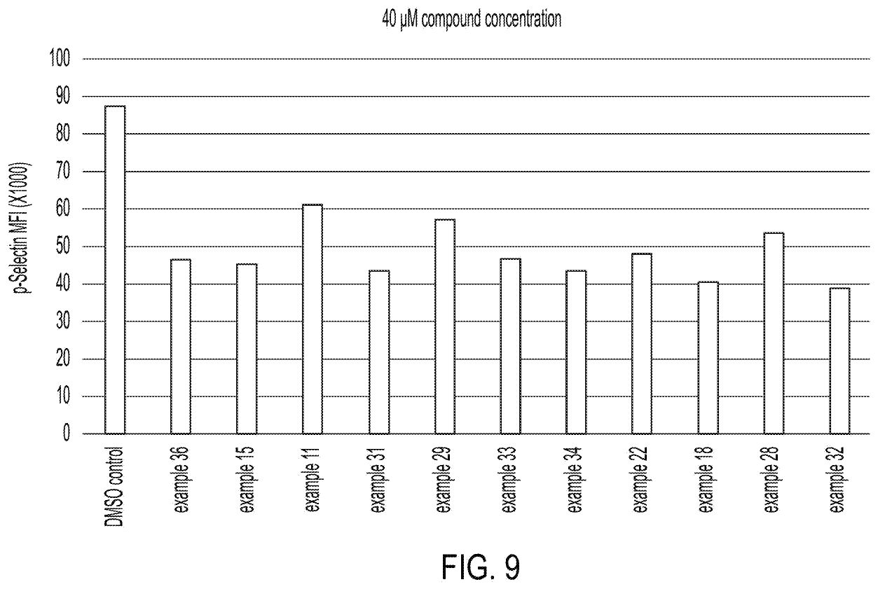

FIGS. 8 and 9 show that in human platelet activation assays, compounds of examples 15, 11, 31, 29, 33, 34, 22, 18, 28 and 32 are inhibitory.

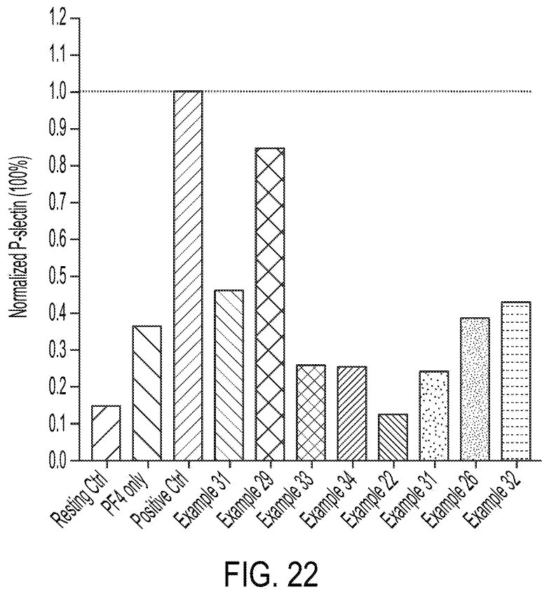

FIG. 22 shows that the compounds of Examples 31, 29, 44, 34, 22, 28, 36 and 32 modulate the activity of normalized P-selectin.

### Experimental Protocol

FcγRIIA transgenic murine blood was drawn from inferior vena cava under anesthesia into a 1 ml syringe with 10% volume of acid citrate dextrose (ACD). Whole blood was diluted with 1× Tyrode buffer (5 mM N-2-hydroxyethylpiperazine-N′-2-ethanesulfonic acid, 0.1% BSA, 134 mM NaCl, 0.3 mM NaH₂PO₄, 3 mM KCl, 2 mM MgCl₂, 12 mM NaHCO₃, 5 mM glucose) with 0.02 U/ml apyrase and 1 μM PGE1 and centrifuged at 100 g for 10 min. Platelet rich plasma (PRP) was carefully removed and centrifuged at 1000 g for 5 min. Platelet pellet was resuspended in Tyrode buffer with 0.02 U/ml apyrase at 2×108/ml concentration at 37° C. PF4 antagonists are diluted in Tyrode buffer, and are incubated with 10 μg/ml human PF4 purified protein at room temperature for 30 min. The PF4-drug complex is then incubated with 1×10⁸/ml platelets with 1 mM CaCl₂, 0.1% BSA at 37° C. for 10 min. 40 μg/ml KKO and 10 μg/ml anti-CD62P (P-selectin) antibodies are added into the reaction. One milliliter ice-cold PBS is added to each reaction after 5 min of incubation. Labelled platelets are then analyzed by a BD Accuri C6 flow cytometer. The results are represented by both the mean fluorescence intensity and by the percentage of positive platelets.

### Example A9—P-Selectin Levels in Human Platelets

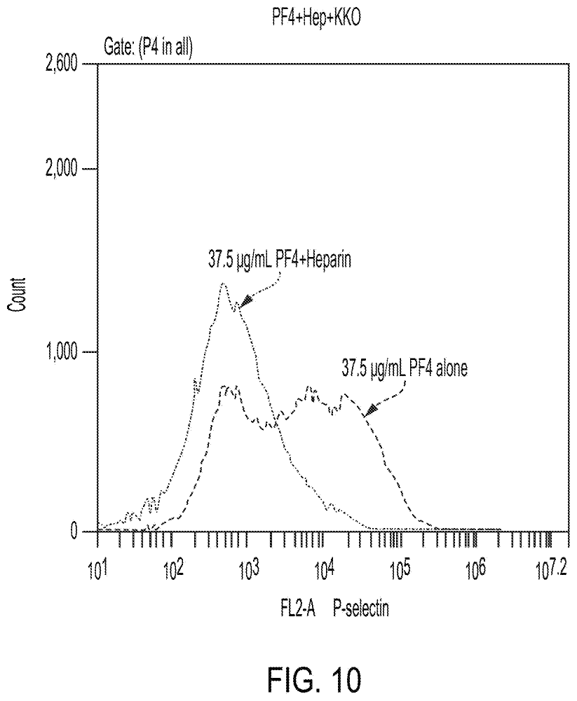

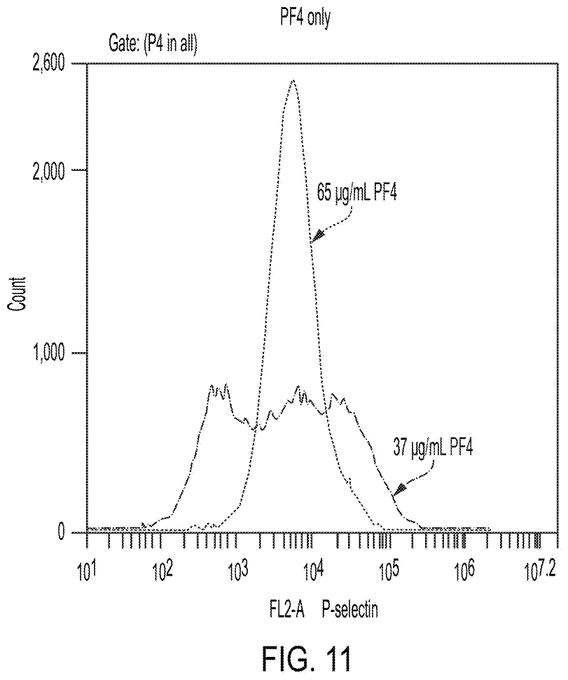

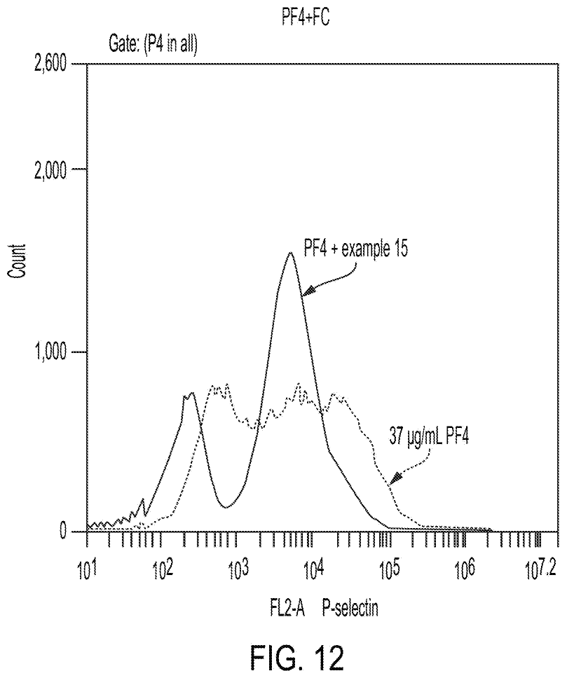

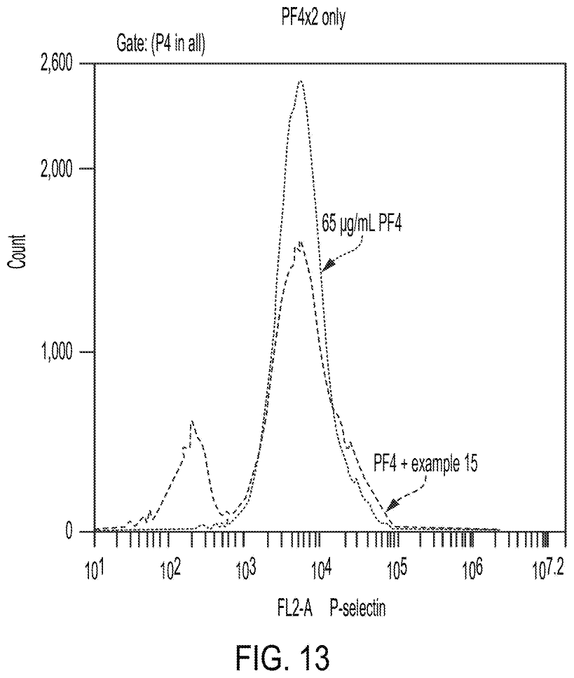

FIGS. 10-13 show effect of PF4 concentration, a combination of PF4 and heparin, and a combination of PF4 and example 15 on P-selectin levels in human platelets.

### Experimental Protocol

For 100 μL reaction, add 2.5×10⁷/ml human platelets, add 1.5 mM CaCl₂, 37.5 μg/ml or 65 μg/ml PF4,10 p/ml heparin (2 μL of 500 U/ml), with or without 100 μM of the exemplified compound (5 μl of 2 mM) for 30 min. Add KKO at 50 μg/ml (2 μL 2.6 mg/ml KKO) all at RT for 20 min. FL2-A is P-selectin.

### Example A10—P-Selectin Levels in the HIT Mouse Model

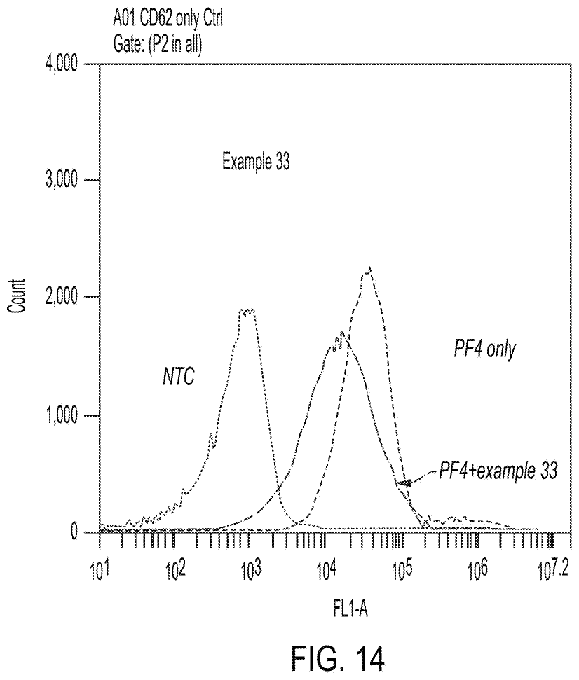

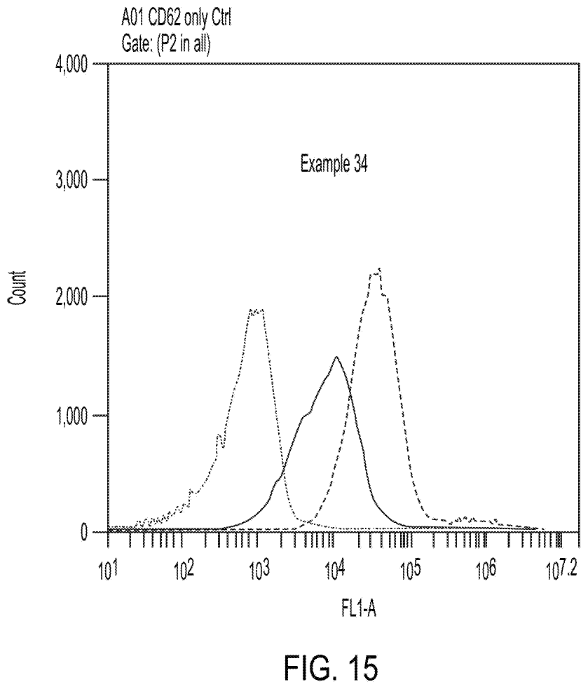

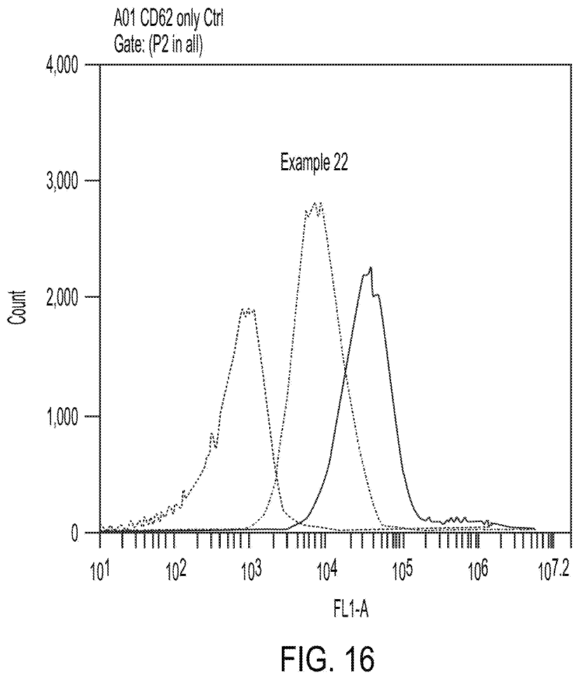

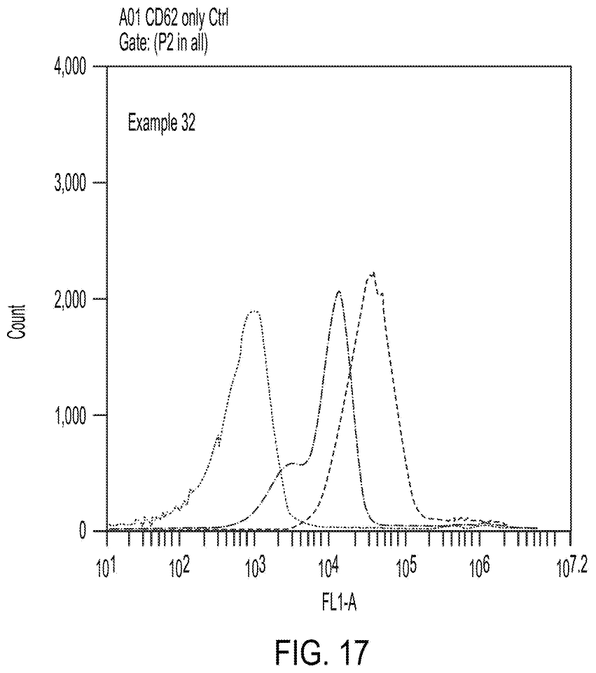

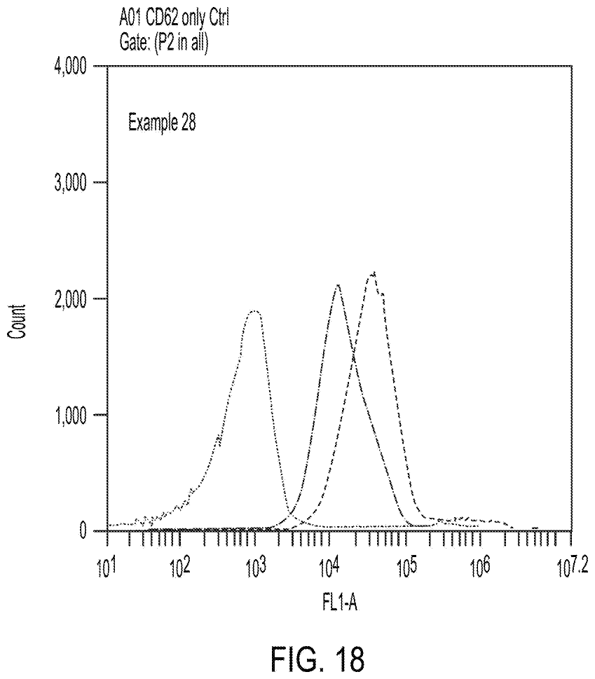

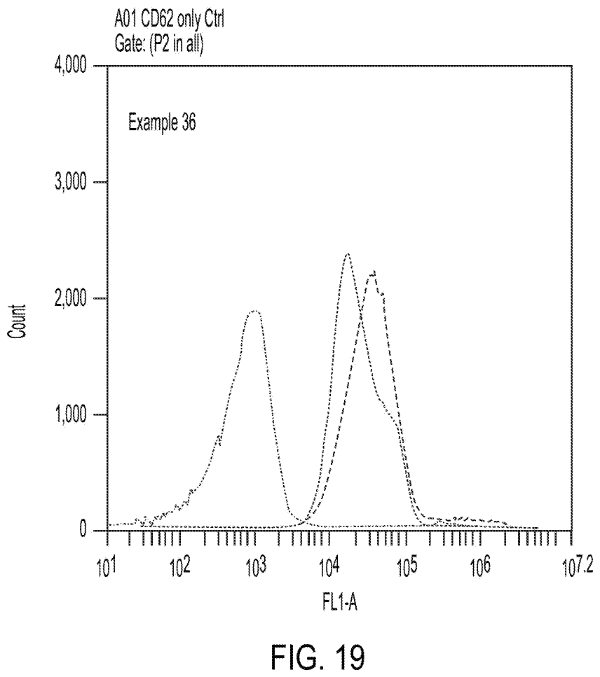

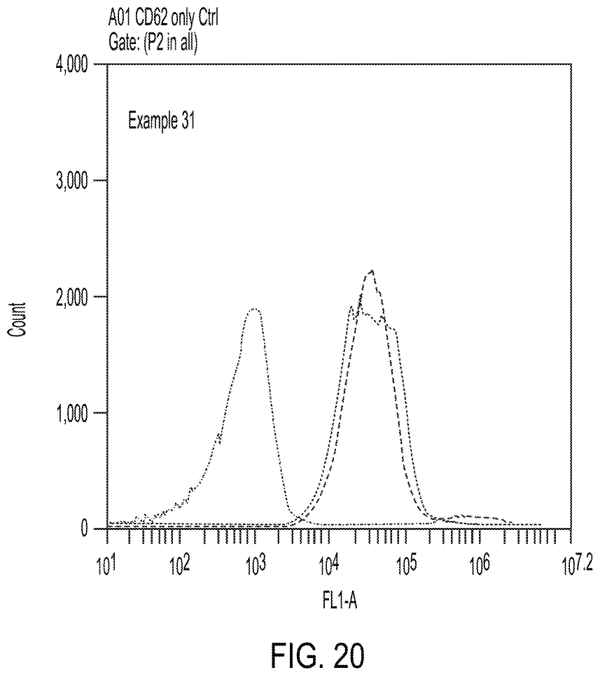

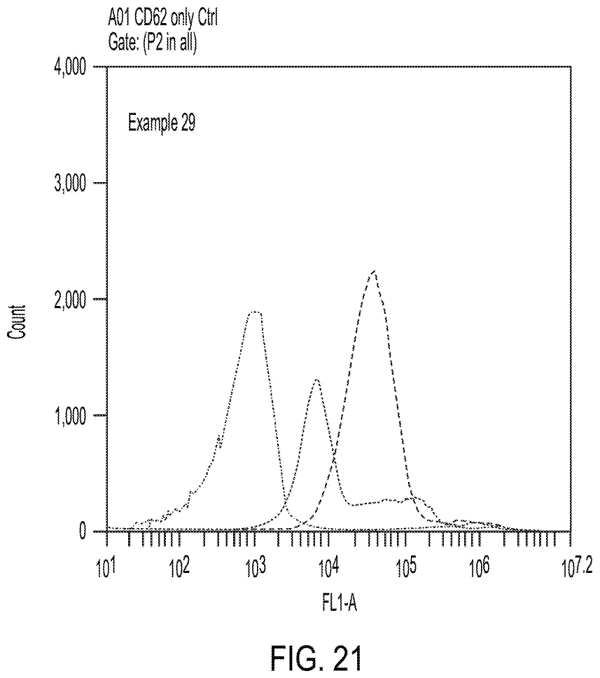

FIGS. 14-21 show efficacy of exemplified compounds 33, 34, 22, 32, 28, 36, 31 and 29 in preventing P-selectin expression in FcγRIIA transgenic murine platelets.

### Experimental Protocol

37.5 μg/ml PF4 with/without 50 μM of the exemplified compound incubated with 3×10⁷/ml platelets; 50 μg/ml KKO.

Unless otherwise indicated, all numbers expressing quantities of ingredients, properties such as molecular weight, reaction conditions, and so forth used in the specification and claims are to be understood as being modified in all instances by the term “about.” As used herein the terms “about” and “approximately” means within 10 to 15%, preferably within 5 to 10%. Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

1. A compound of Formula (I): 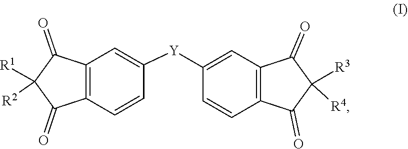
or a pharmaceutically acceptable salt thereof, wherein:
Y is selected from O, C(═O), S(═O)₂, C(R⁵)(R⁶), C≡C, and a bond between the indane-1,3-dione rings of the compound of Formula (I);
R¹ and R³ are each independently selected from H, halo, CN, C₁₋₆ alkoxycarbonyl, C₁₋₆ alkylcarbonyl, C(O)NH(R^{a1}), and C(O)Cy⁴, wherein C₁₋₆ alkyl in the C₁₋₆ alkylcarbonyl is optionally substituted with 1, 2, or 3 substituents independently selected from Cy⁴, amino, C₁₋₆ alkoxy, C₁₋₆ cycloalkoxy, C₆₋₁₀ arylcarbonyl, C₁₋₃ alkoxy-C₁₋₃ alkoxy, carboxy, C₁₋₃ alkoxycarbonyl, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl, and halo;
R² and R⁴ are each independently selected from H, C₁₋₆ alkoxycarbonyl, and C₁₋₆ alkylcarbonyl;
R⁵ and R⁶ are each C₁₋₃ haloalkyl;
each R^{a1} is independently selected from C₁₋₆ alkyl, C₁₋₆ alkenyl, Cy¹, C₁₋₆ alkoxycarbonyl, and S(O)₂R^{a2}, wherein said C₁₋₆ alkyl is optionally substituted with 1, 2, or 3 substituents independently selected from Cy², carboxyl, C₁₋₃ alkoxycarbonyl, C₁₋₃ alkoxy, C₁₋₃ alkoxy-C₁₋₃ alkoxy, and C₁₋₃ haloalkoxy;
each Cy¹ is independently selected from C₃₋₁₀ cycloalkyl, 4-10 membered heterocycloalkyl, C₆₋₁₀ aryl, and 5-10 membered heteroaryl, each of which is optionally substituted with 1, 2, or 3 substituents independently selected from halo, NO₂, C₁₋₆ alkyl, C₁₋₃ alkoxy, C₁₋₆ alkoxycarbonyl, C₁₋₆ alkylcarbonyl, and Cy³;
each Cy² is independently selected from C₆₋₁₀ aryl, C₃₋₁₀ cycloalkyl, 4-10 membered heterocycloalkyl, and 5-10 membered heteroaryl, each of which is optionally substituted with 1, 2, or 3 substituents independently selected from halo, C₁₋₃ alkyl, C₁₋₃ alkoxy, and C₁₋₃ haloalkoxy;
each Cy³ is independently selected from C₆₋₁₀ aryl and 5-10 membered heteroaryl;
each Cy⁴ is independently selected from C₆₋₁₀ aryl, C₃₋₁₀ cycloalkyl, 4-10 membered heterocycloalkyl, and 5-10 membered heteroaryl, each of which is optionally substituted with 1, 2, or 3 substituents independently selected from halo, aryl, carboxy, C₁₋₆ alkyl, C₁₋₆ alkoxy, C₁₋₆ alkoxycarbonyl, C₁₋₆ alkylidene, and oxo; wherein C₁₋₆ alkoxy in said C₁₋₆ alkoxycarbonyl is optionally substituted with C₆₋₁₀ aryl; and
each R^{a2} is C₆₋₁₀ aryl, optionally substituted with C₁₋₃ alkyl,
with a proviso that the compound of Formula (I) is not any one of the compounds selected from:

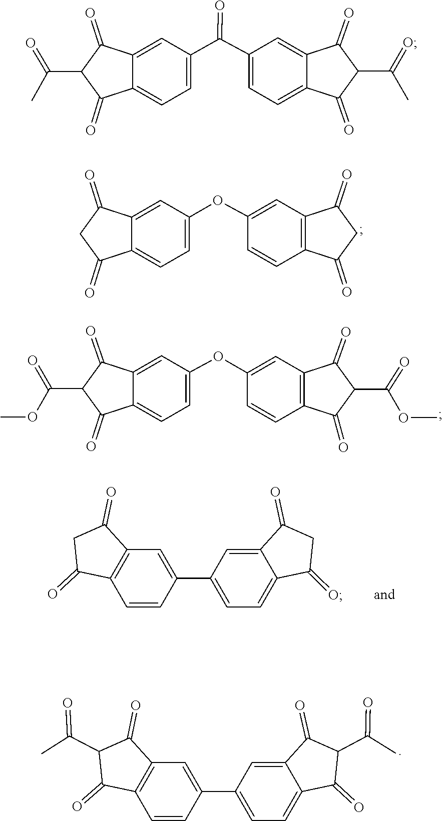

2. (canceled)

3. The compound of claim 1, wherein Y is C(═O).

4.-8. (canceled)

9. The compound of claim 1, wherein:
R² and R⁴ are each independently selected from C₁₋₆ alkoxycarbonyl and C₁₋₆ alkylcarbonyl; and
R¹ and R³ are each independently selected from C₁₋₆ alkoxycarbonyl and C₁₋₆ alkylcarbonyl.

10. The compound of claim 1, wherein:
R¹ and R² are each H; and
R³ and R⁴ are each independently selected from C₁₋₆ alkoxycarbonyl and C₁₋₆ alkylcarbonyl.

11.-25. (canceled)

26. The compound of claim 1, wherein the compound of Formula (I) has Formula (Ia): 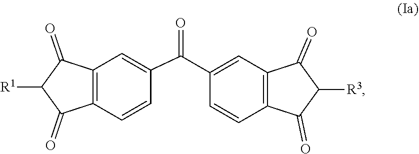
or a pharmaceutically acceptable salt thereof.

27.-35. (canceled)

36. The compound of claim 26, wherein R¹ and R³ are each independently C₁₋₆ alkoxycarbonyl.

37. The compound of claim 26, wherein R¹ and R³ are each independently an C₁₋₆ alkylcarbonyl, wherein C₁₋₆ alkyl in the C₁₋₆ alkylcarbonyl group is optionally substituted with 1, 2, or 3 substituents independently selected from Cy⁴, amino, C₁₋₆ alkoxy, C₁₋₆ cycloalkoxy, C₆₋₁₀ arylcarbonyl, carboxy, C₁₋₃ alkoxycarbonyl, C₆₋₁₀ aryloxy, —N(C₁₋₃ alkyl)(C₁₋₆ alkoxycarbonyl), —NH(C₁₋₆ alkoxycarbonyl), and —NH(C₁₋₆ alkylcarbonyl), wherein C₁₋₆ alkoxy in any of said C₁₋₆ alkoxycarbonyl groups is optionally substituted with C₆₋₁₀ aryl, wherein C₆₋₁₀ arylcarbonyl may be optionally substituted with 1, 2, or 3 substituents independently selected from C₁₋₃ alkyl, and halo.

38. The compound of claim 37, wherein R¹ and R³ are each independently selected from: —C(O)methyl, —C(O)ethyl, —C(O)propyl, —C(O)butyl, —C(O)hexyl, wherein said methyl, ethyl, propyl, butyl, and hexyl are each optionally substituted with 1, 2, or 3 substituents independently selected from: Cy⁴, amino, methoxy, t-butoxy, carboxy, (methoxy)carbonyl, phenoxy, —N(methyl)C(O)(t-butoxy), —NHC(O)(t-butoxy), —NHC(O)(benzoxy), —NH(acetyl), —NHC(O)(pentyl), and —NHC(O)(isopropyl).

39. The compound of claim 26, wherein R¹ and R³ are each independently C(O)Cy⁴.

40.-42. (canceled)

43. The compound of claim 26, wherein each Cy⁴ is optionally substituted with 1, 2, or 3 substituents independently selected from (t-butoxy)carbonyl, fluoro, (methoxy)carbonyl, methyl, (benzyloxy)carbonyl, methylene, (isopropoxy)carbonyl, methoxy, carboxy, and oxo.

44.-65. (canceled)

66. The compound of claim 1, wherein the compound of Formula (I) is selected from any one of the following compounds: 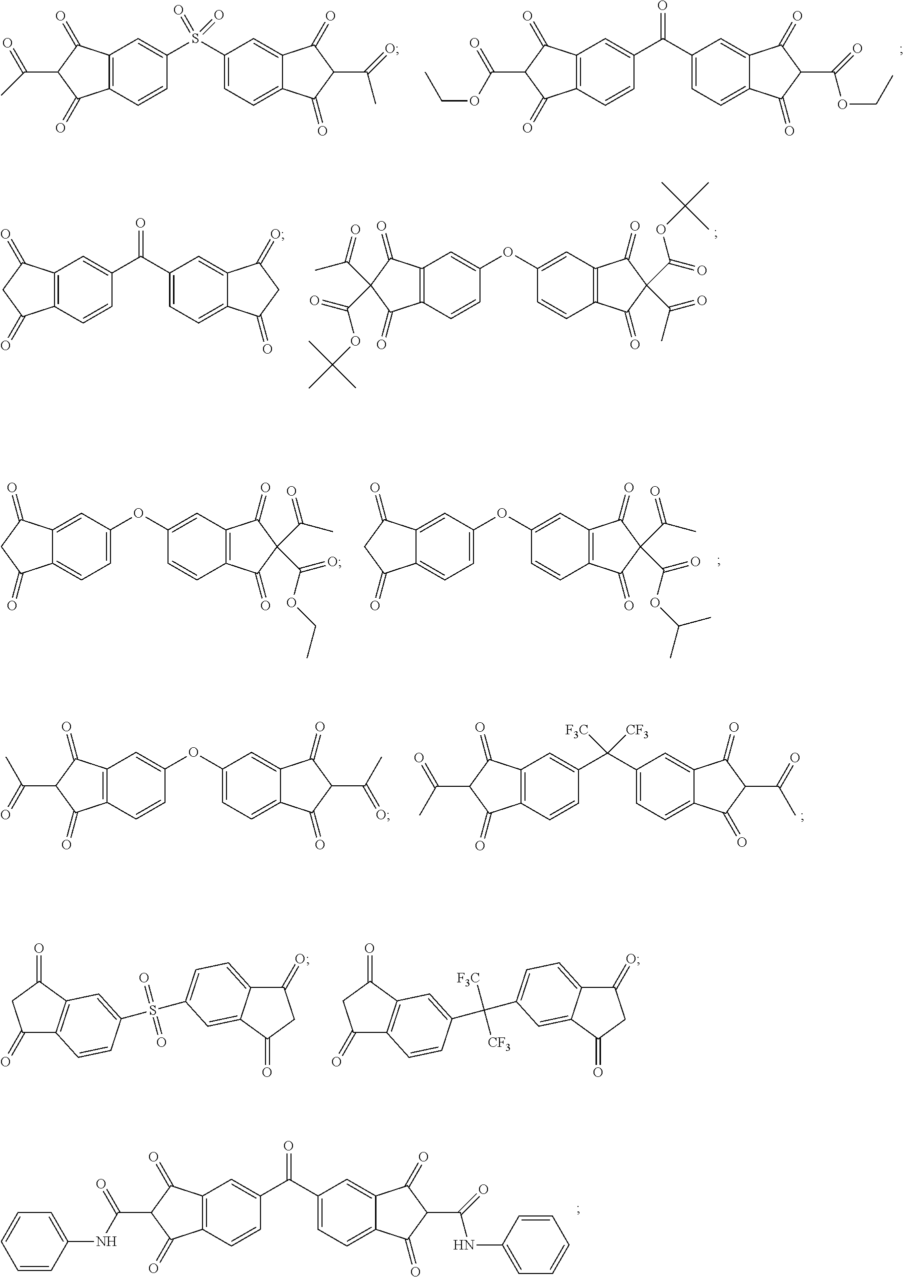 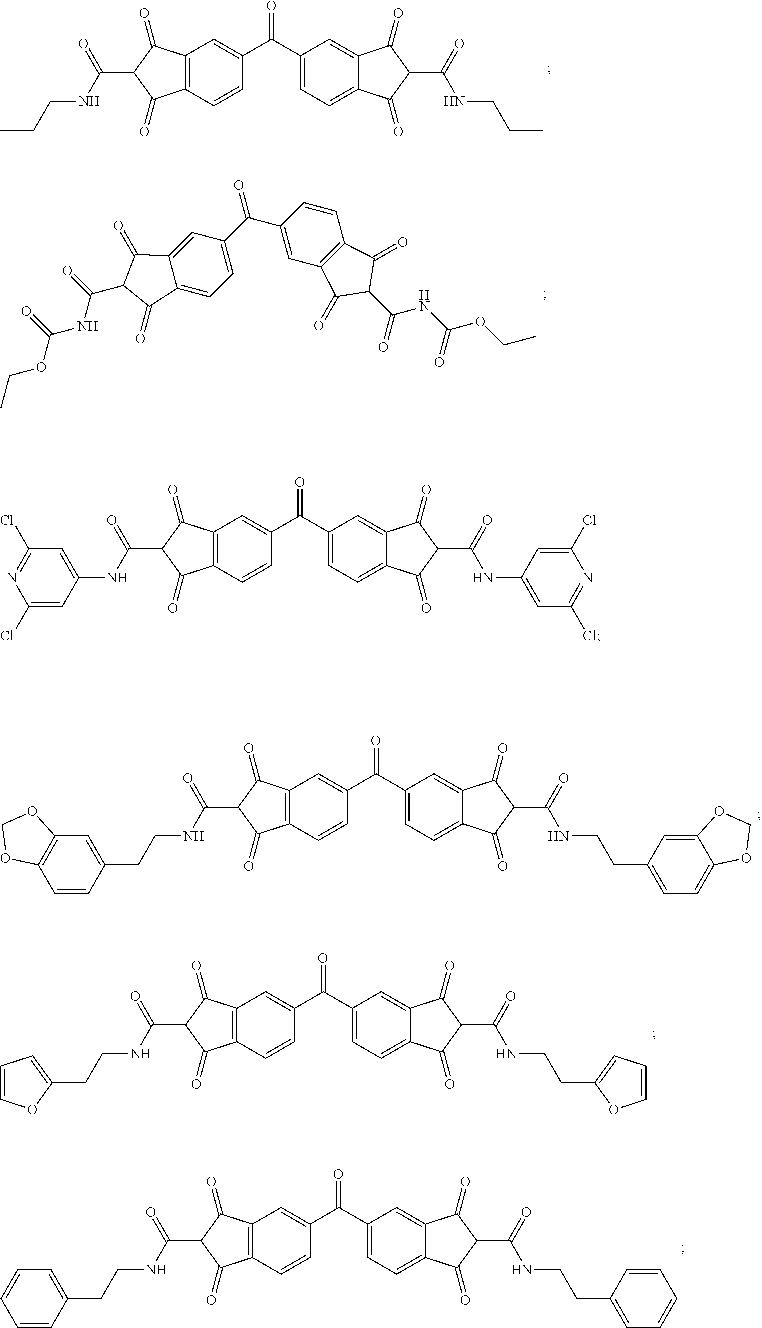 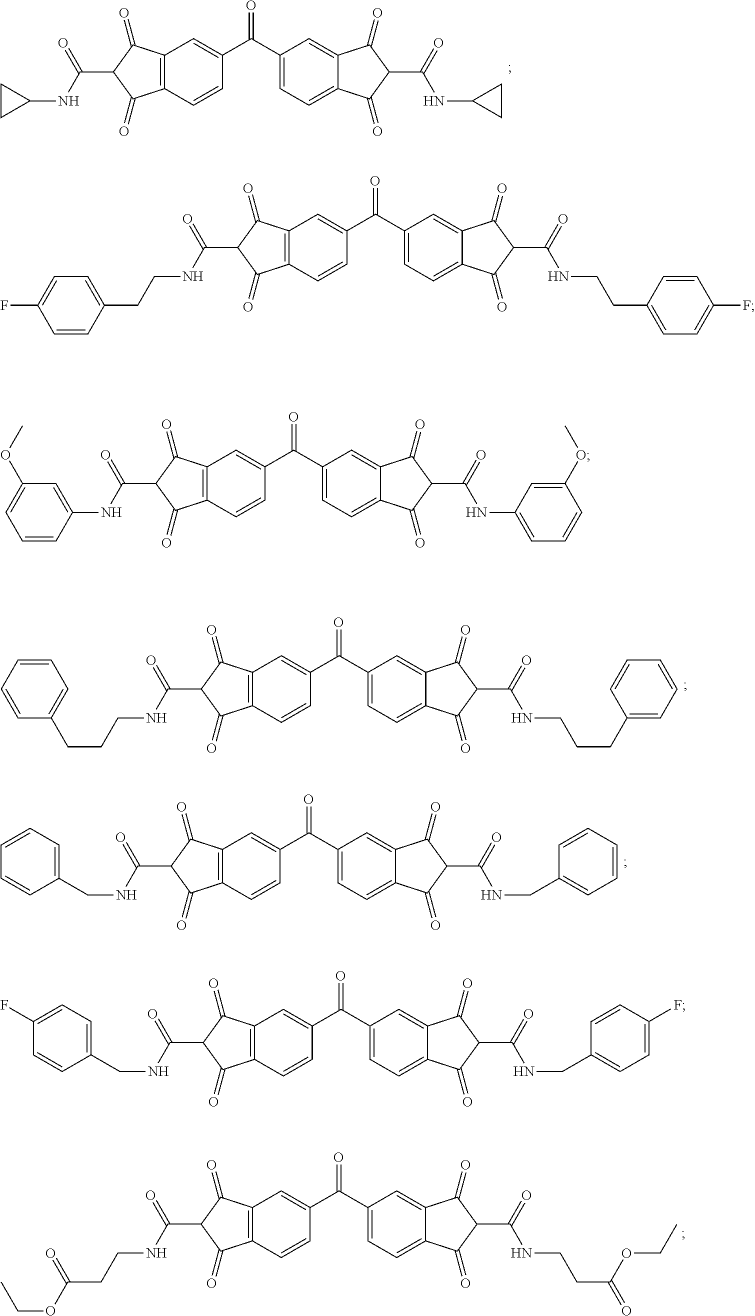 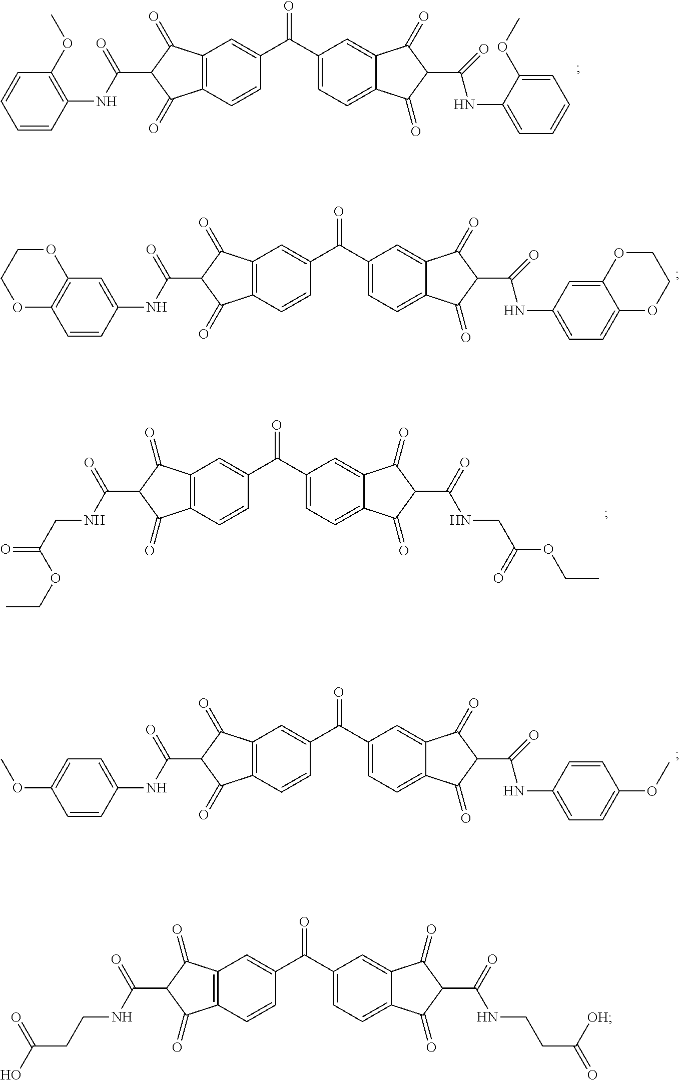 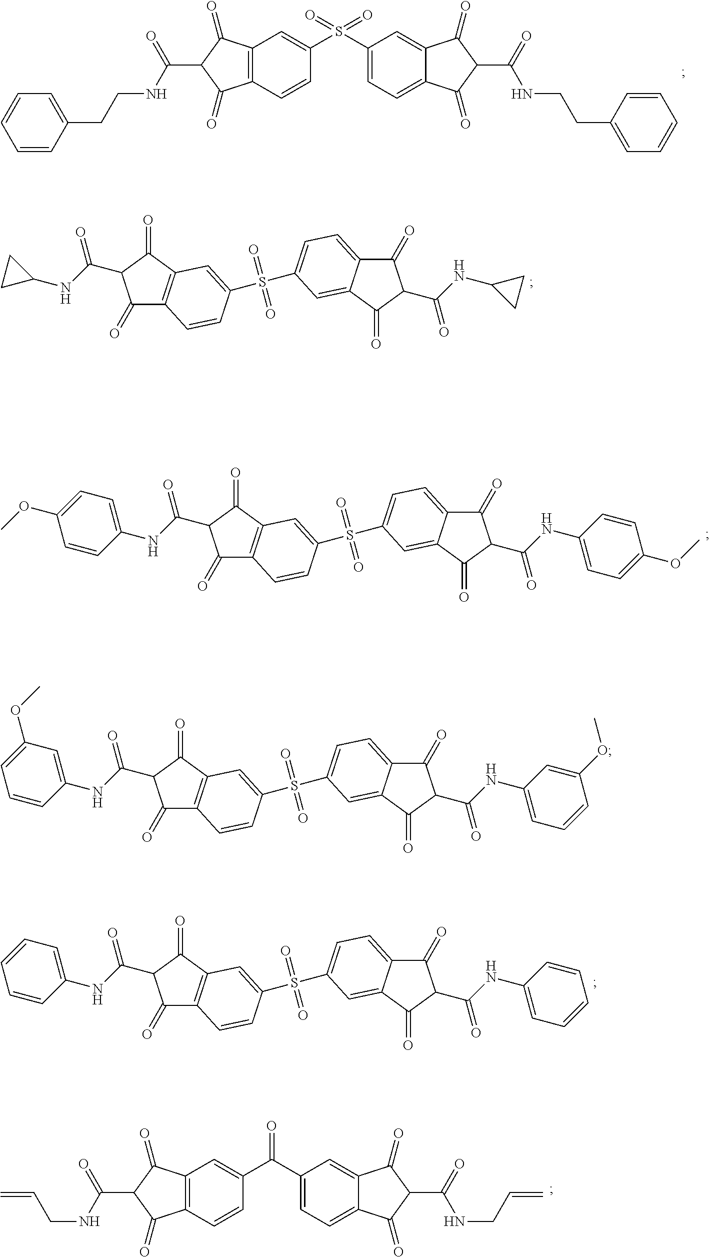 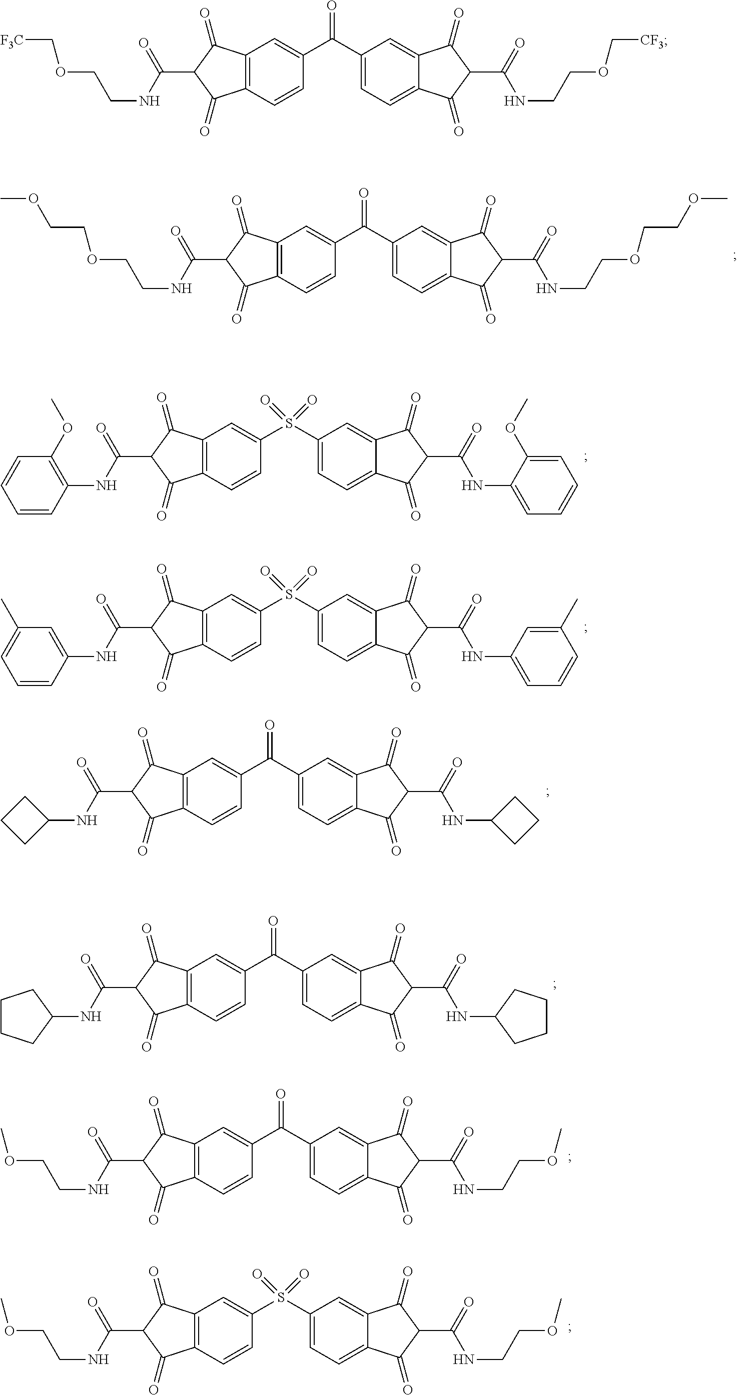 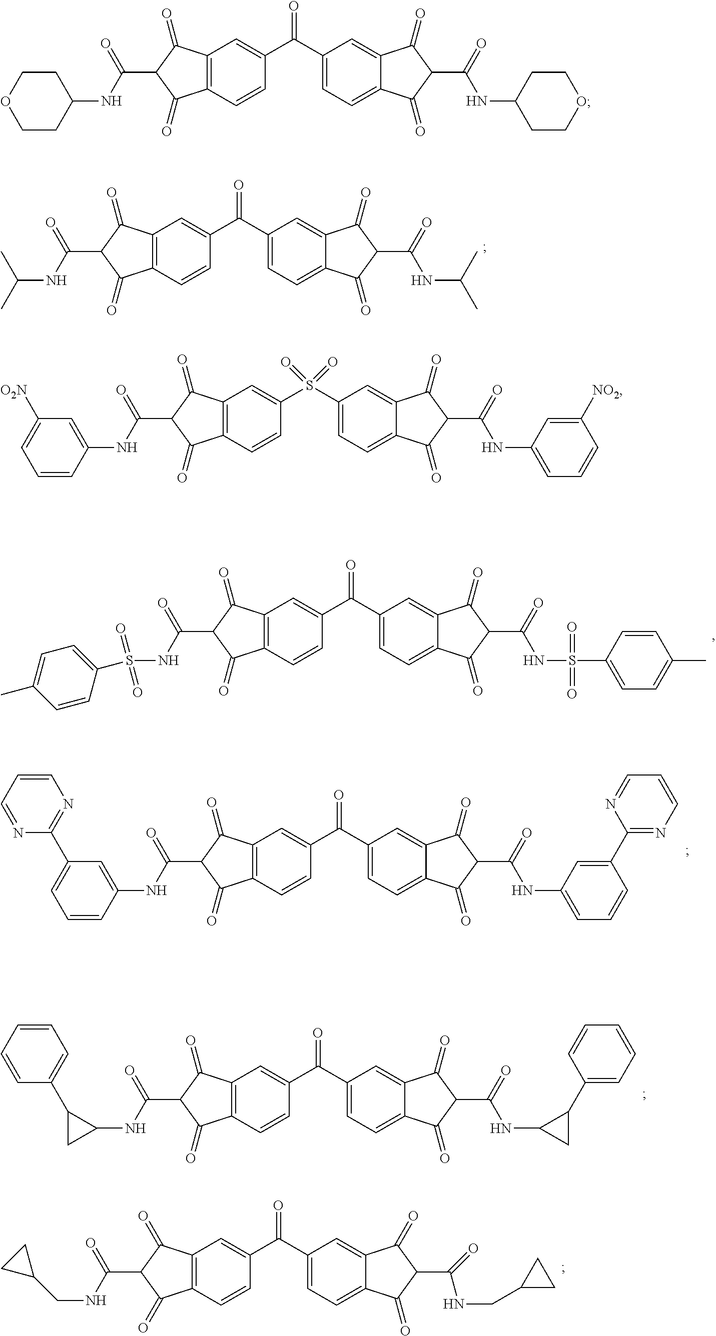 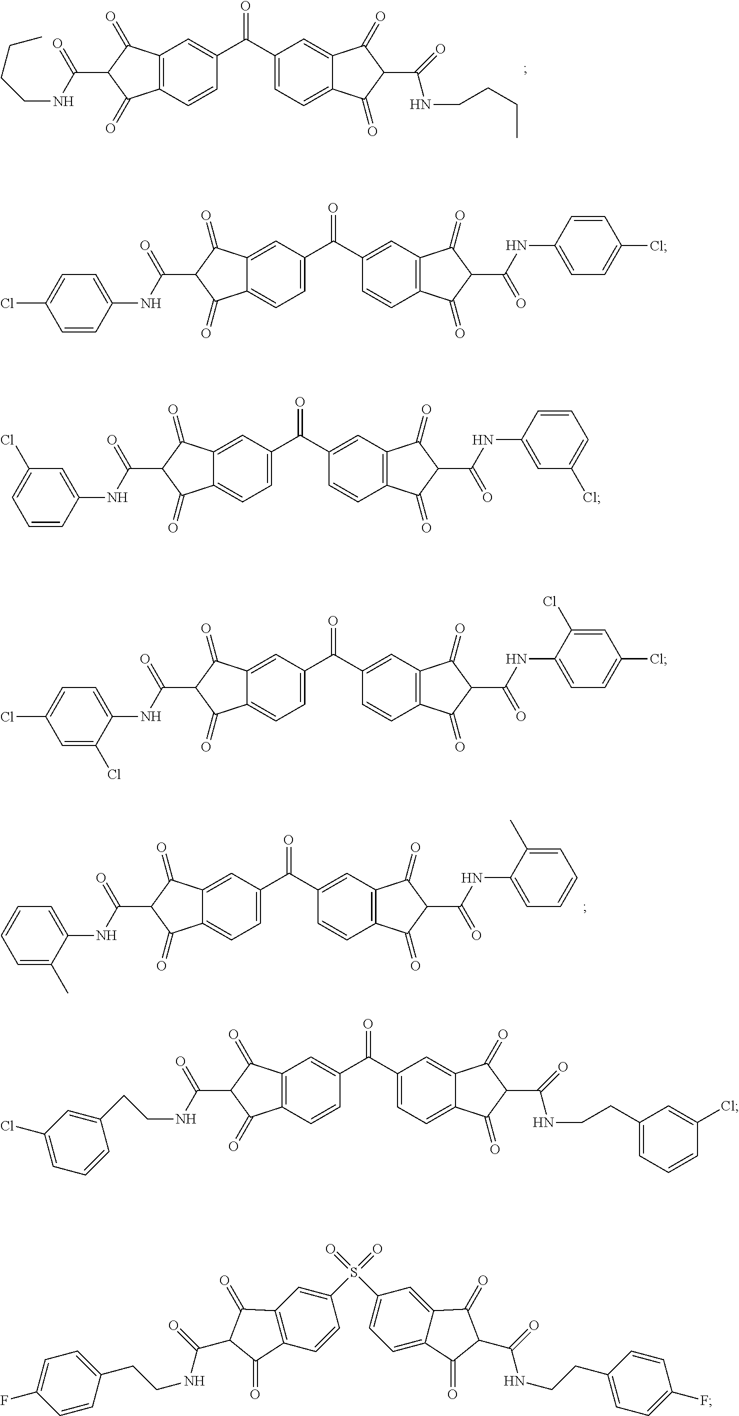 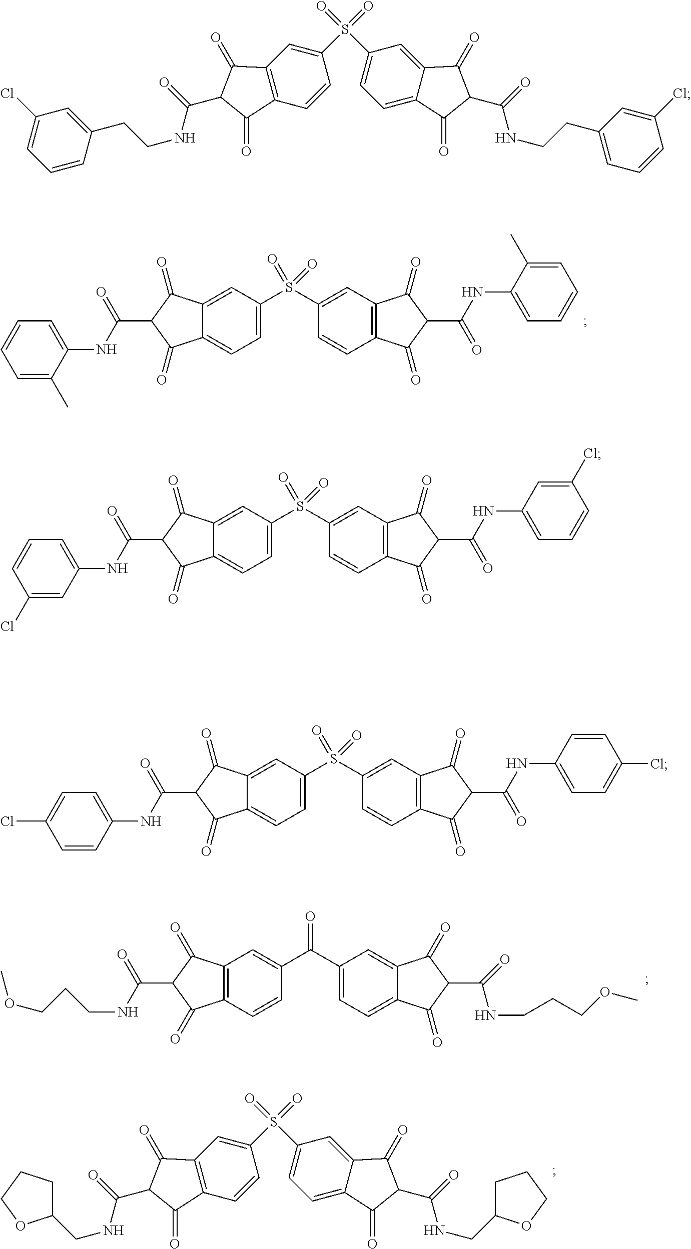 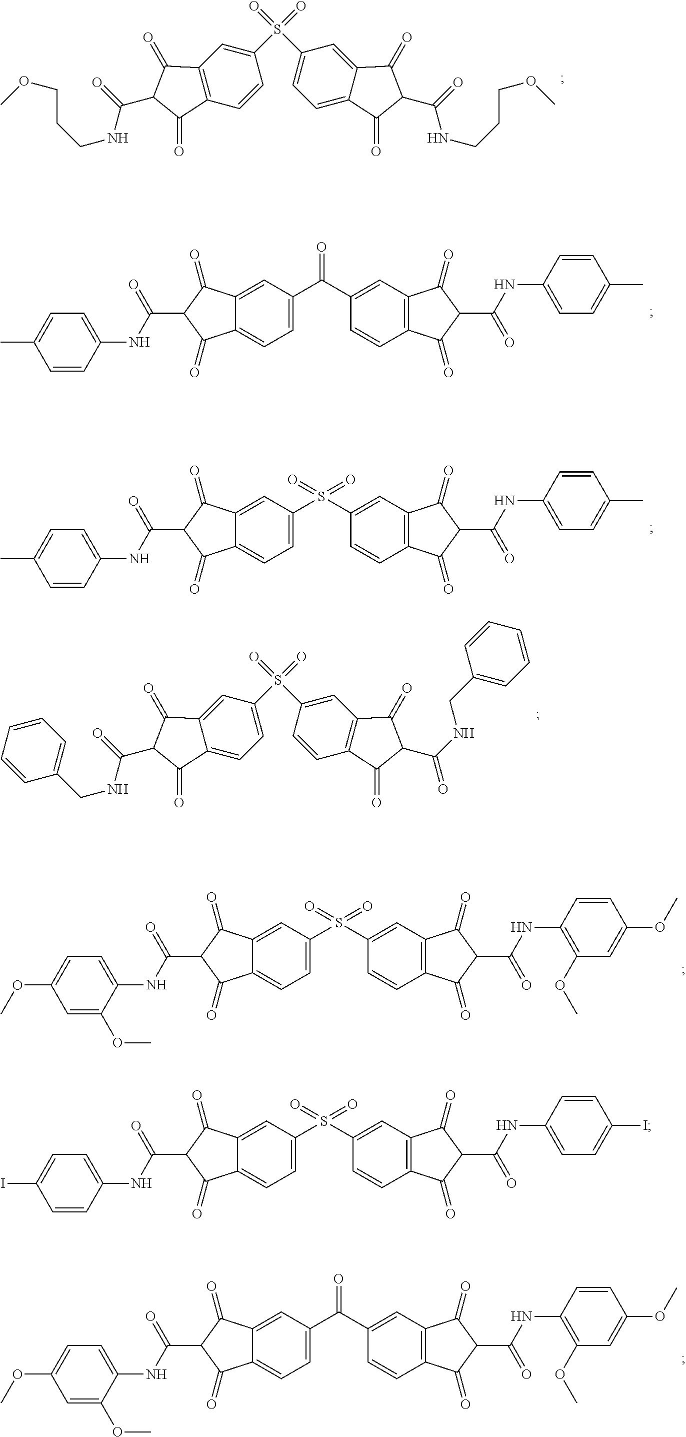 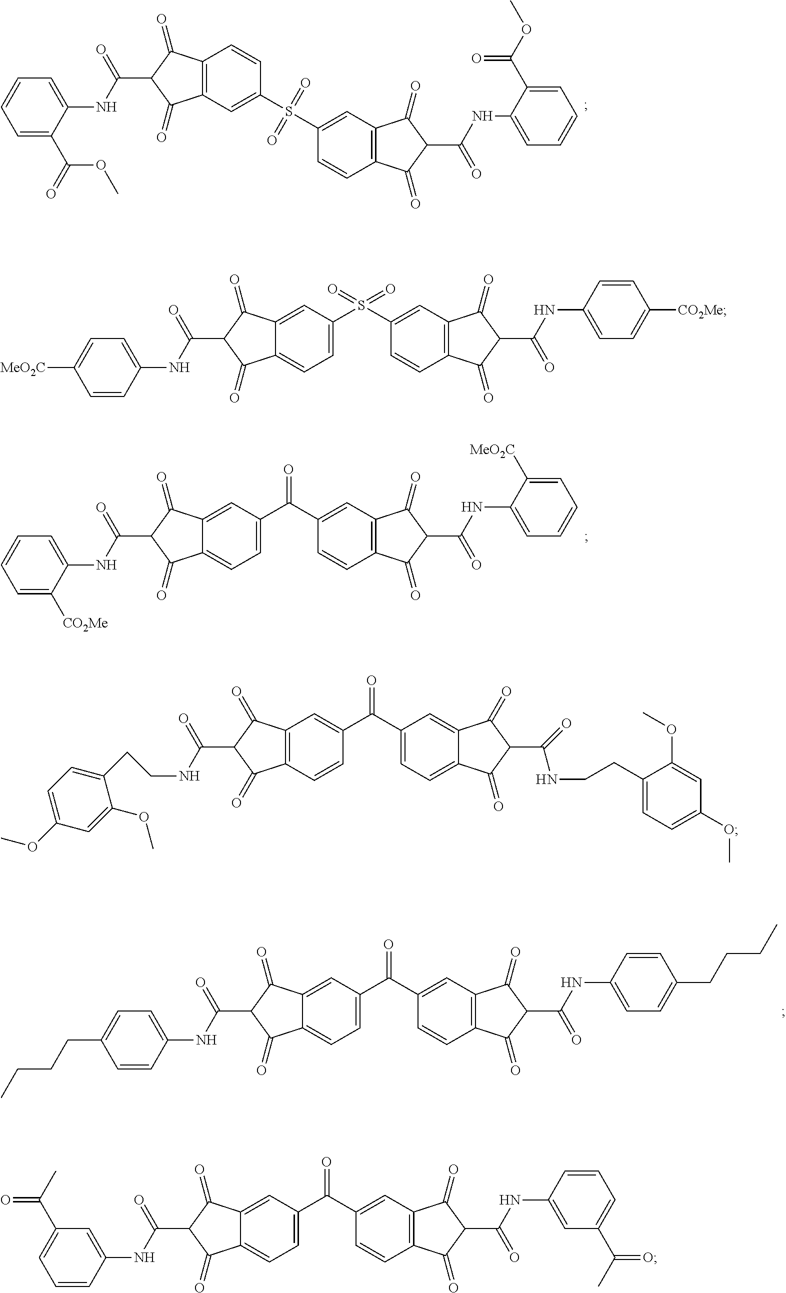 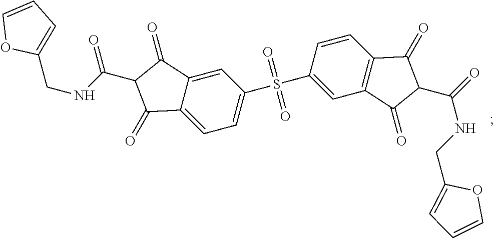 [SMILES: O=C(NCc1ccco1)C1C(=O)c2ccc(S(=O)(=O)c3ccc4c(c3)C(=O)C(C(=O)NCc3ccco3)C4=O)cc2C1=O] 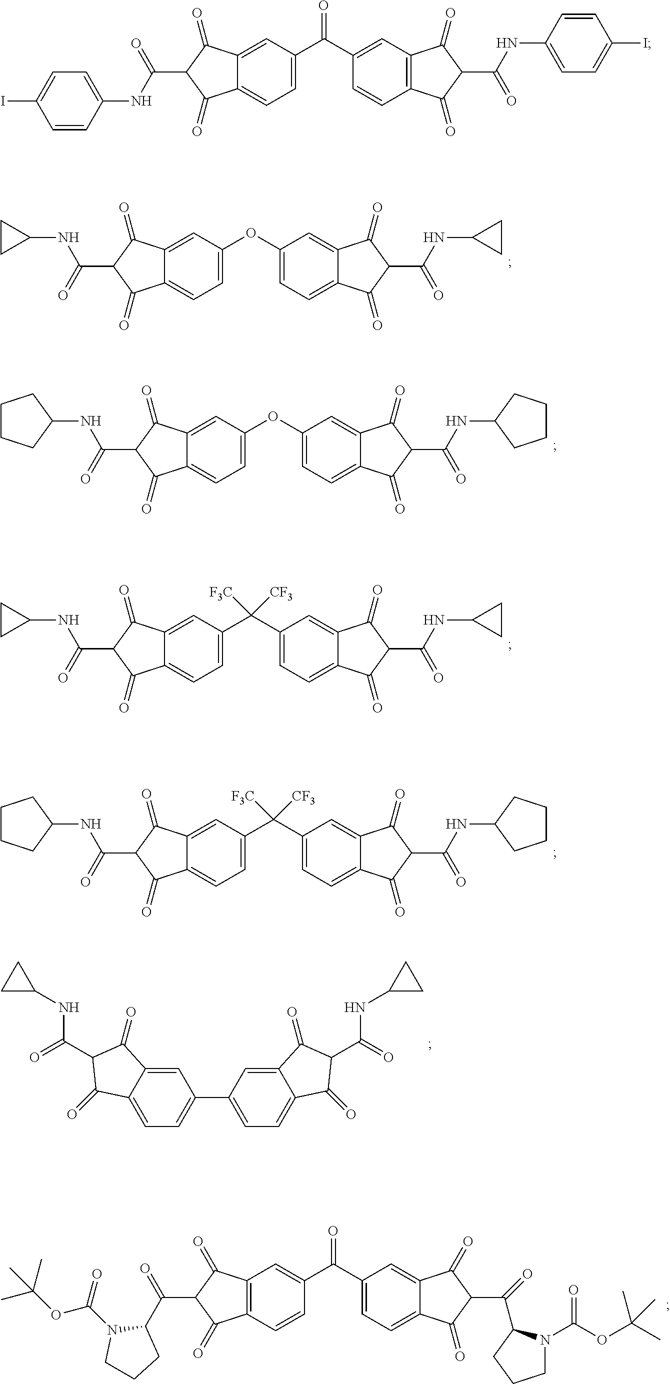 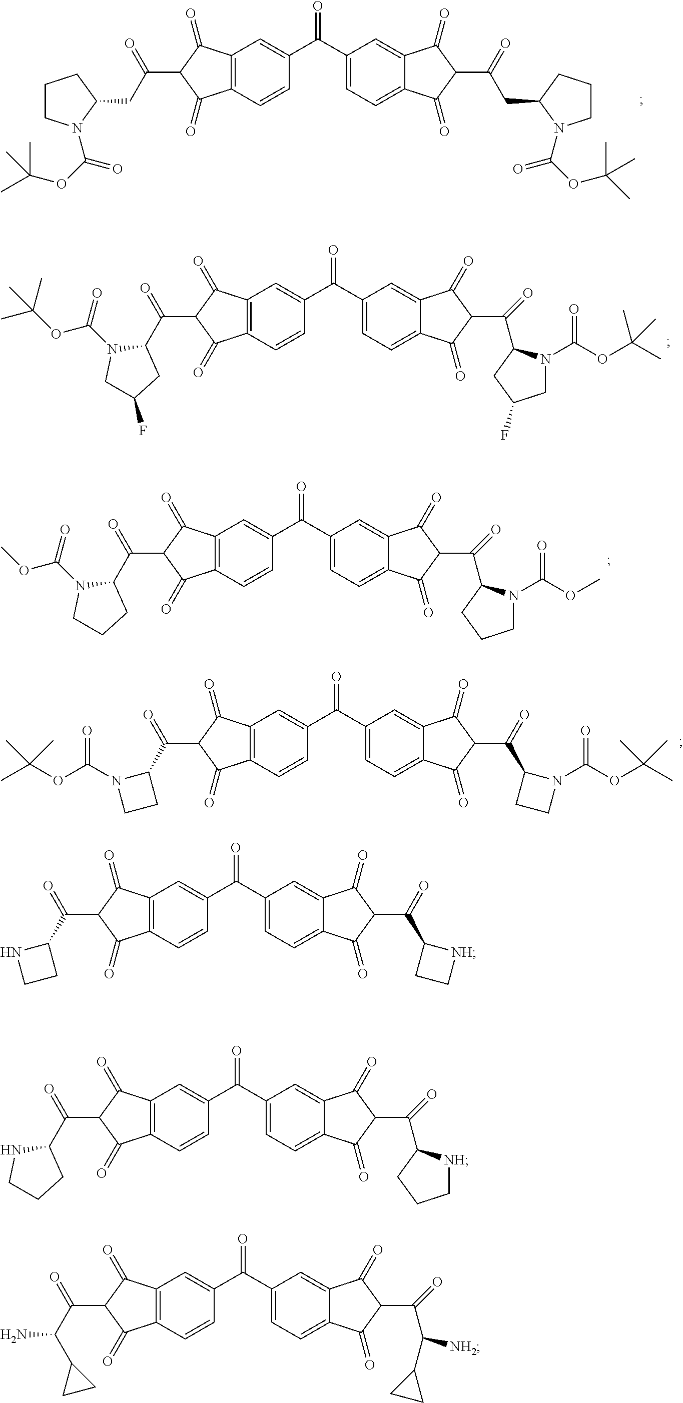 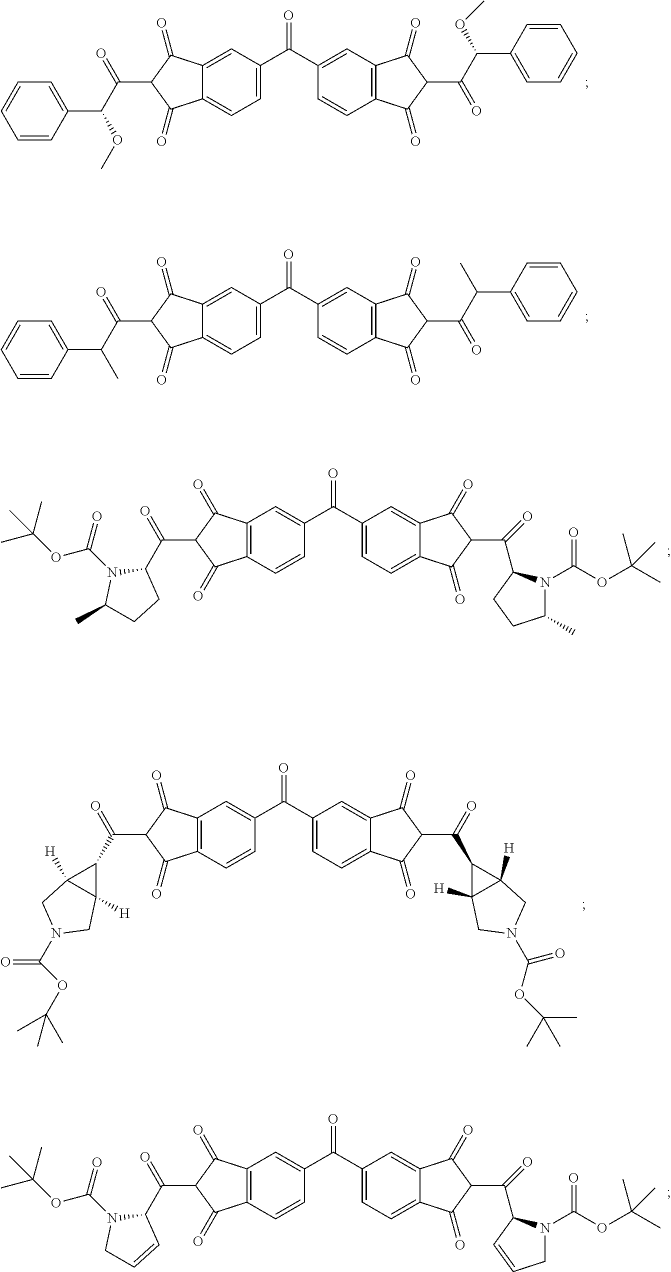 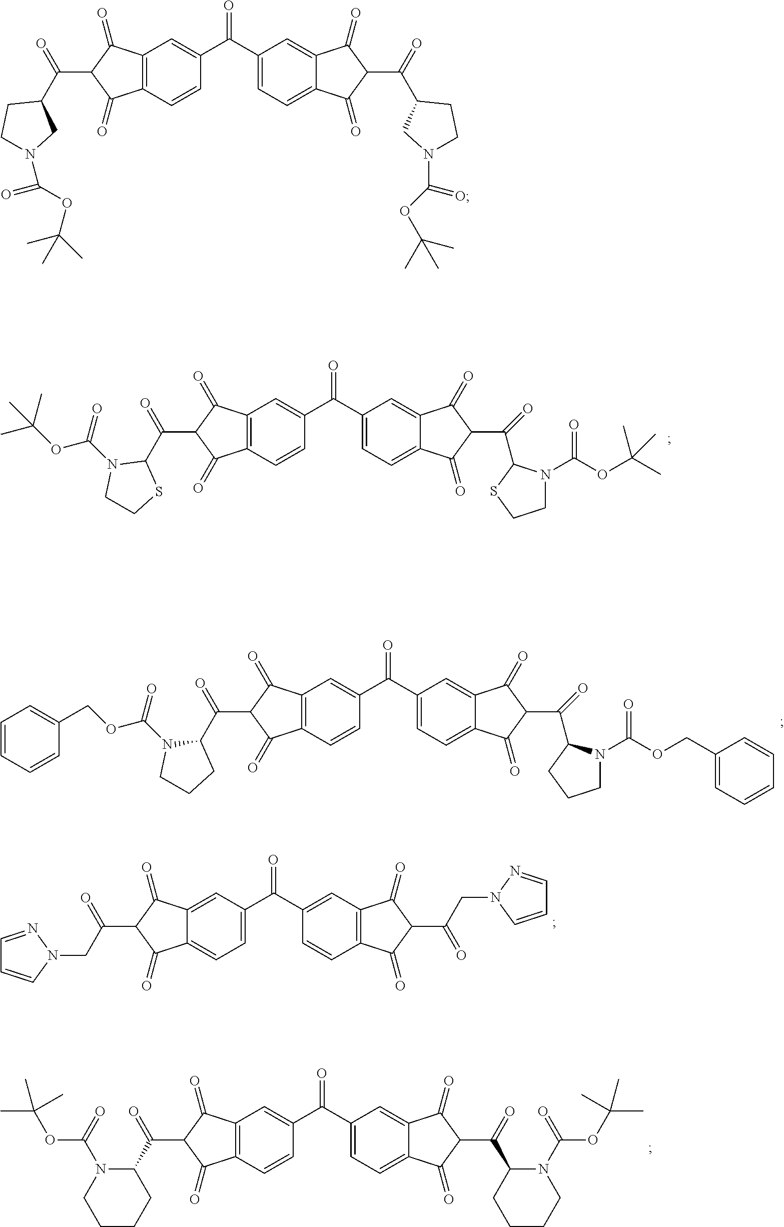 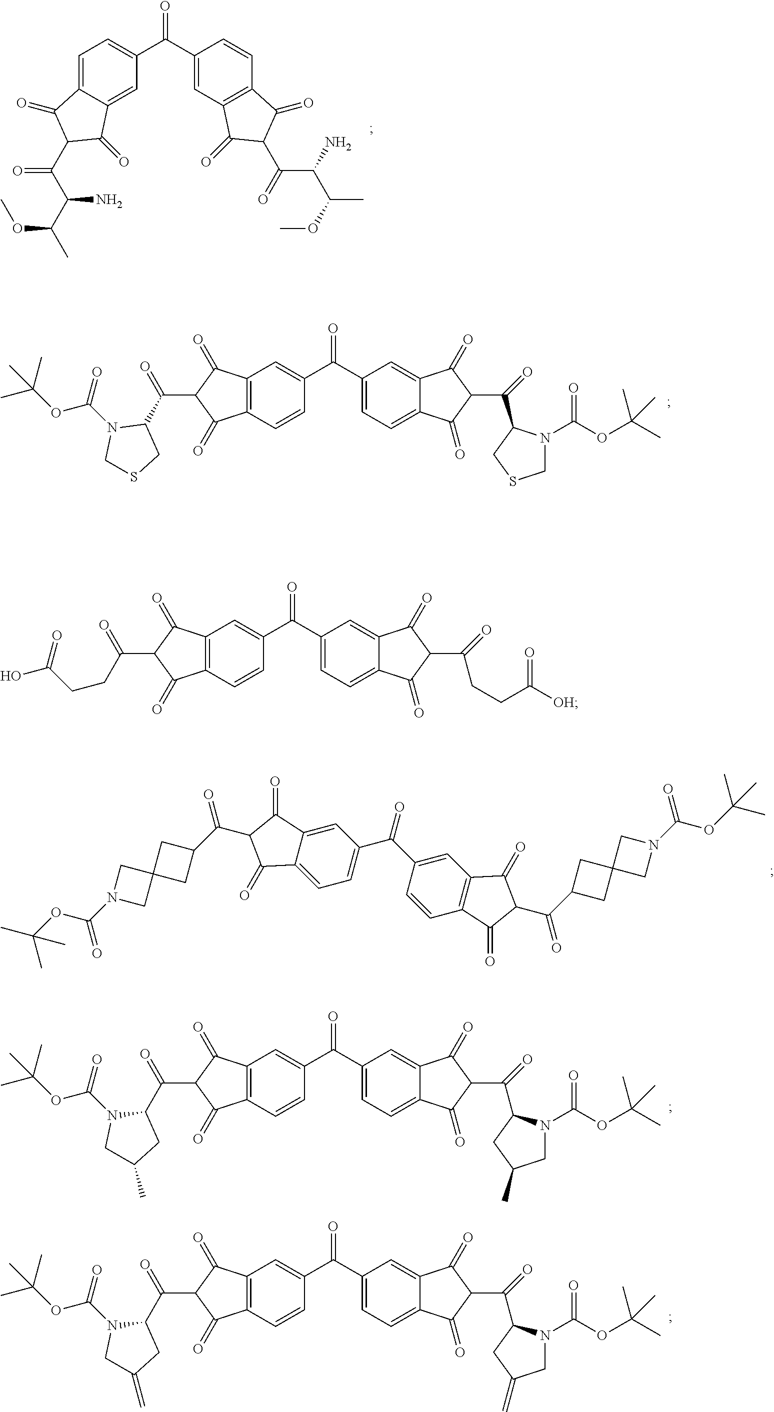 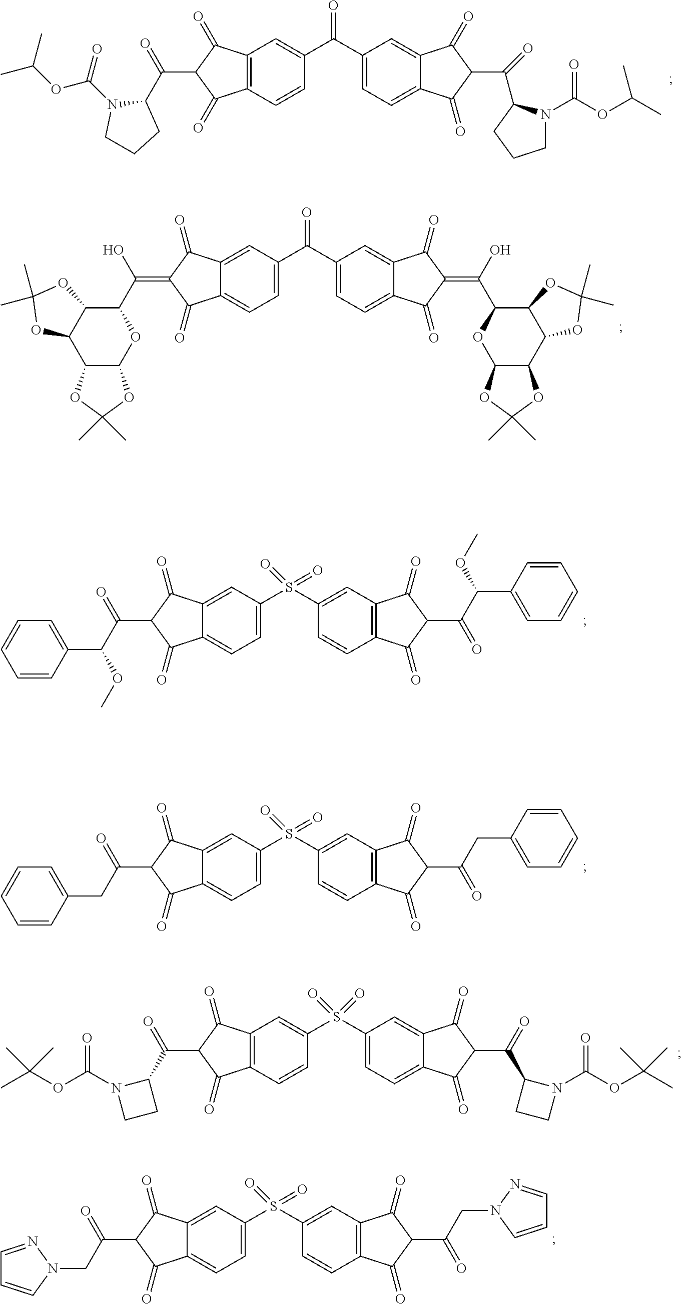 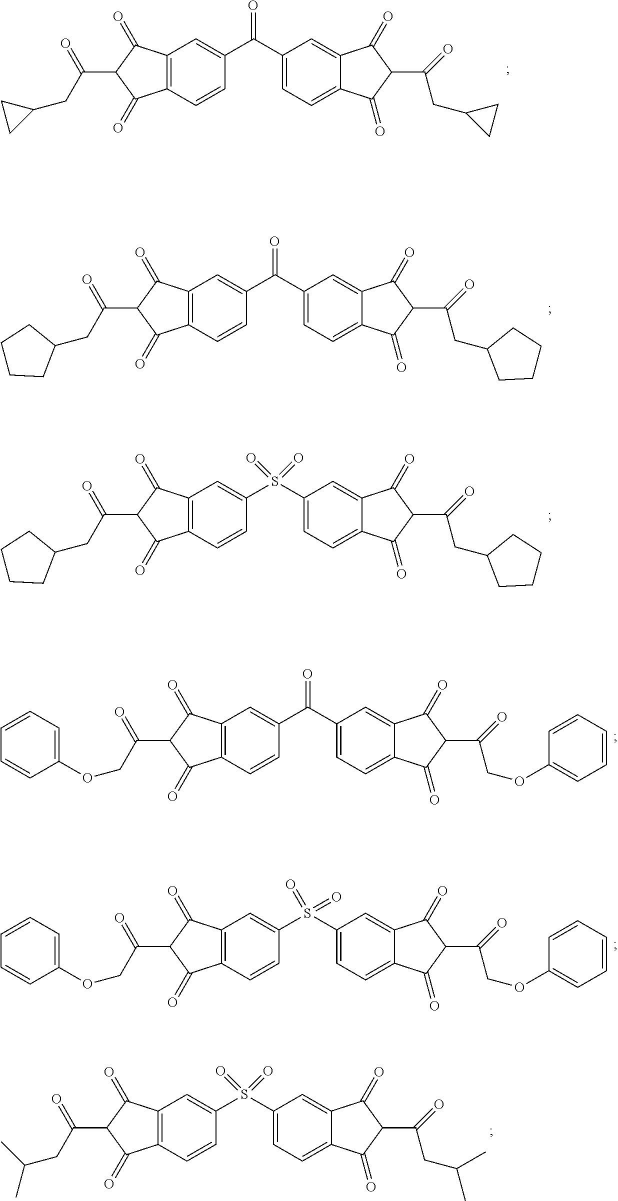 [SMILES: O=C(c1ccc2c(c1)C(=O)C(C(=O)CC1CC1)C2=O)c1ccc2c(c1)C(=O)C(C(=O)CC1CC1)C2=O ; O=C(c1ccc2c(c1)C(=O)C(C(=O)CC1CCCC1)C2=O)c1ccc2c(c1)C(=O)C(C(=O)CC1CCCC1)C2=O ; O=C(CC1CCCC1)C1C(=O)c2ccc(S(=O)(=O)c3ccc4c(c3)C(=O)C(C(=O)CC3CCCC3)C4=O)cc2C1=O ; O=C(c1ccc2c(c1)C(=O)C(C(=O)COc1ccccc1)C2=O)c1ccc2c(c1)C(=O)C(C(=O)COc1ccccc1)C2=O ; O=C(COc1ccccc1)C1C(=O)c2ccc(S(=O)(=O)c3ccc4c(c3)C(=O)C(C(=O)COc3ccccc3)C4=O)cc2C1=O ; CC(C)CC(=O)C1C(=O)c2ccc(S(=O)(=O)c3ccc4c(c3)C(=O)C(C(=O)CC(C)C)C4=O)cc2C1=O] 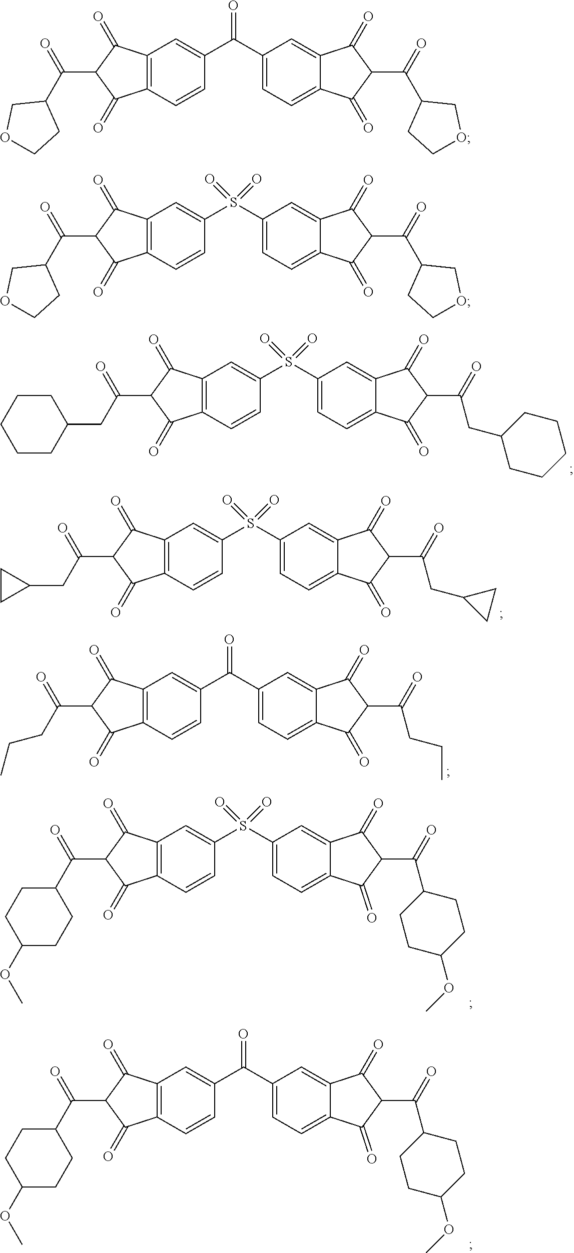 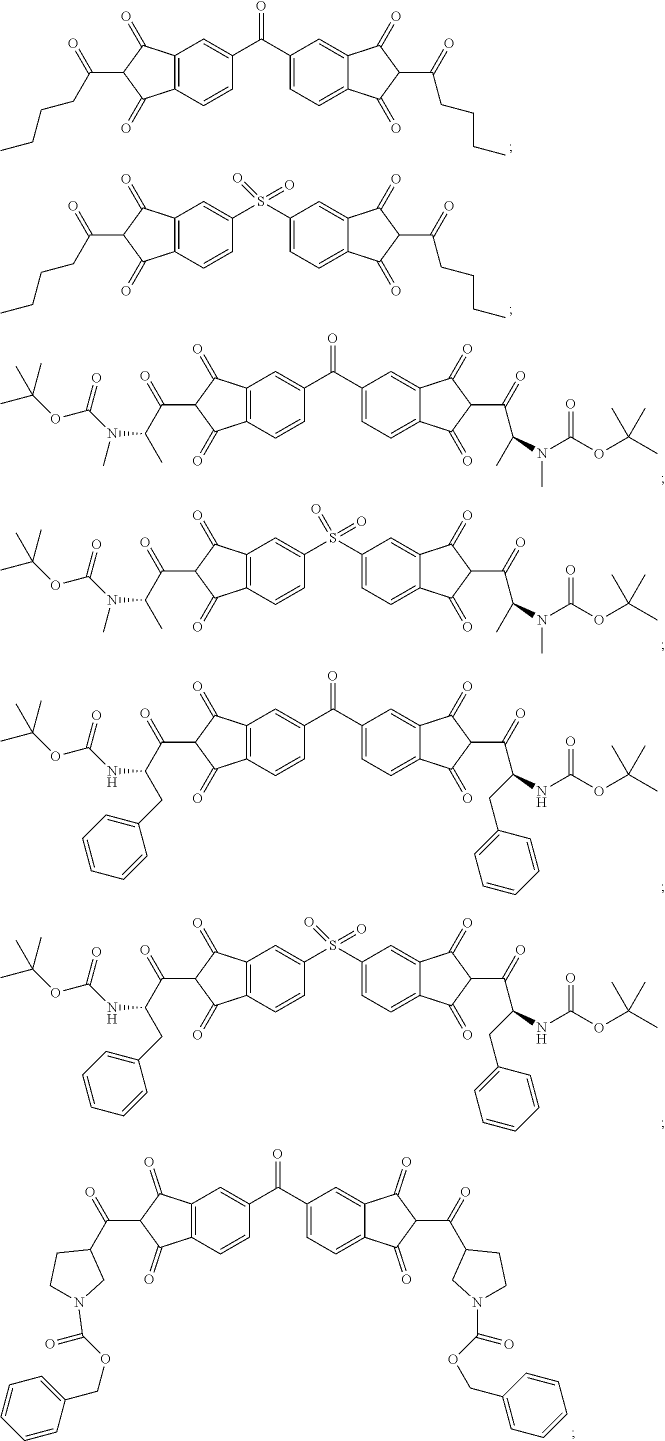 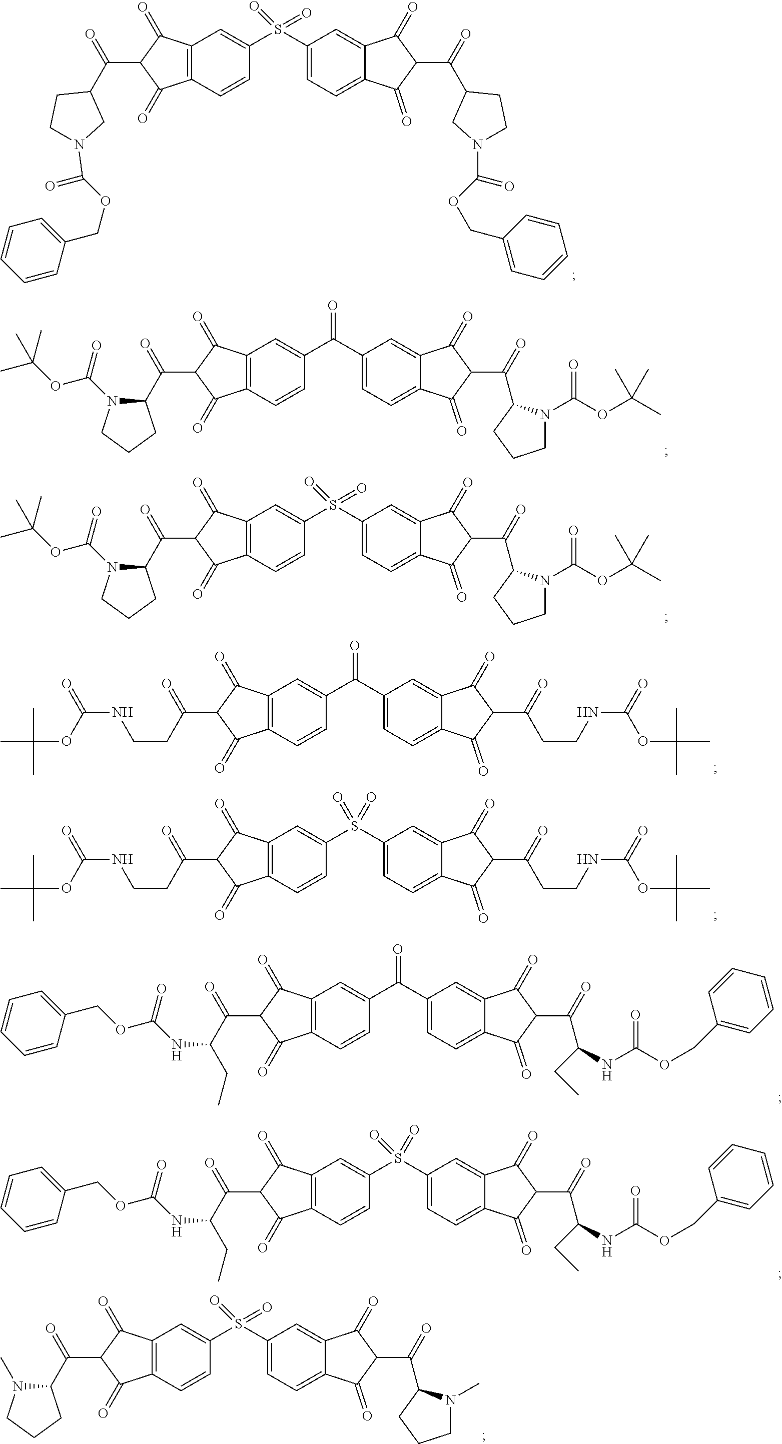 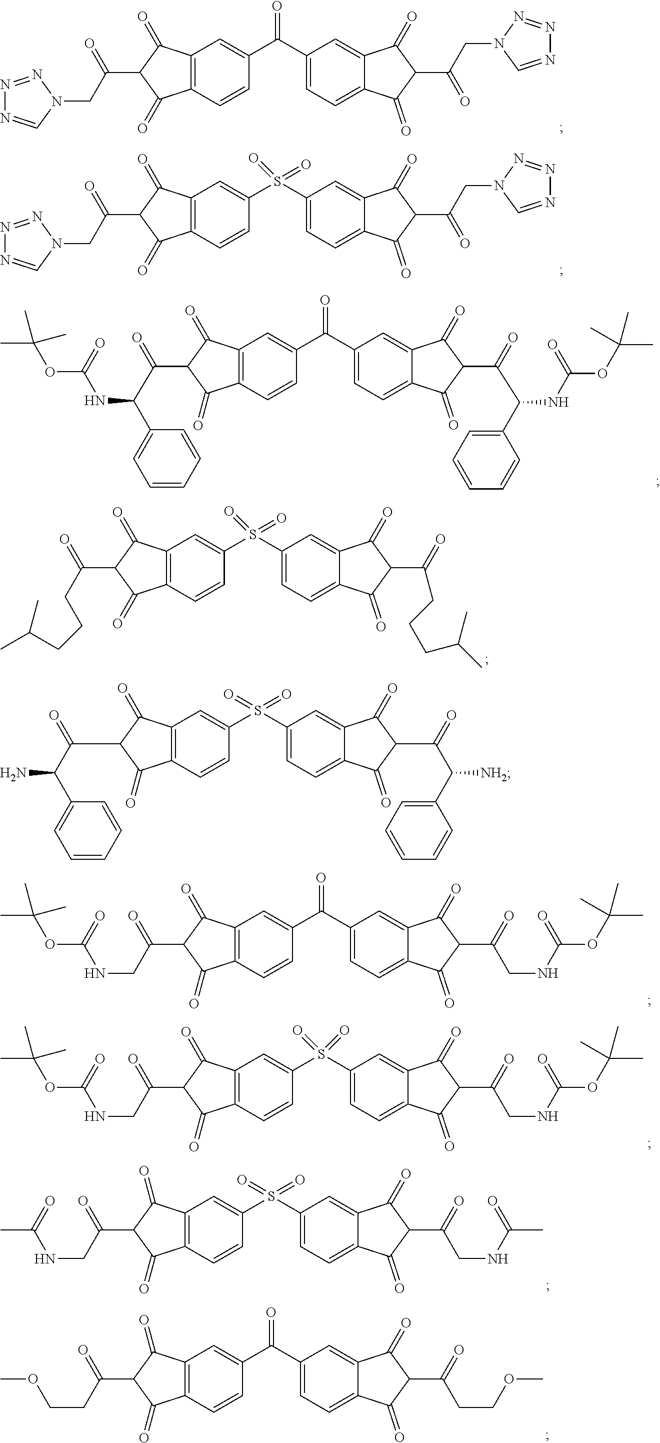 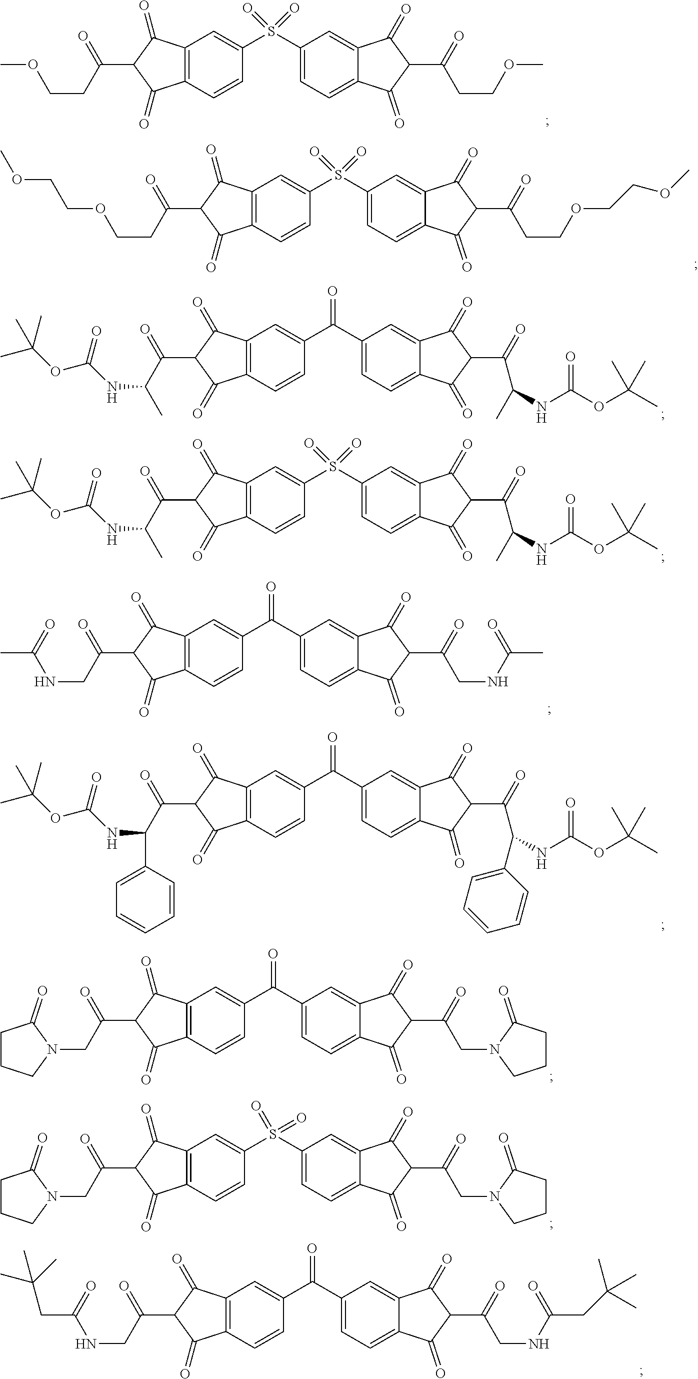 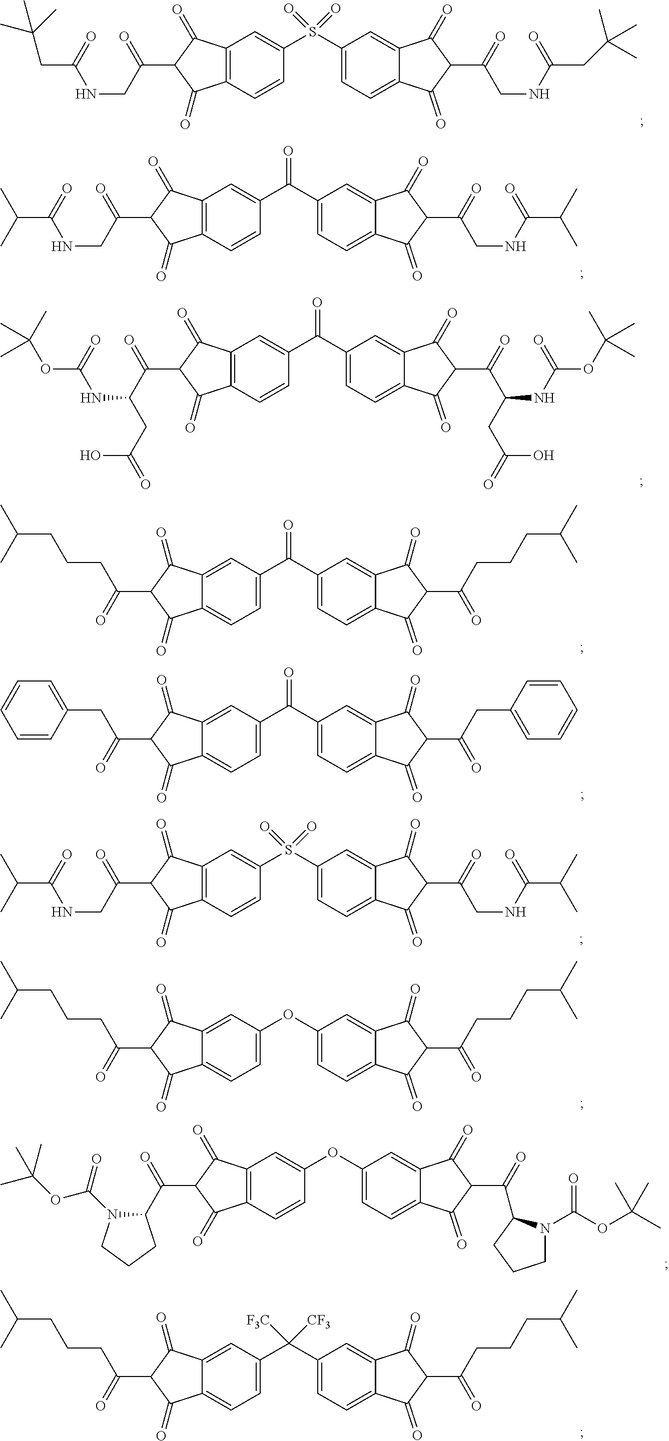 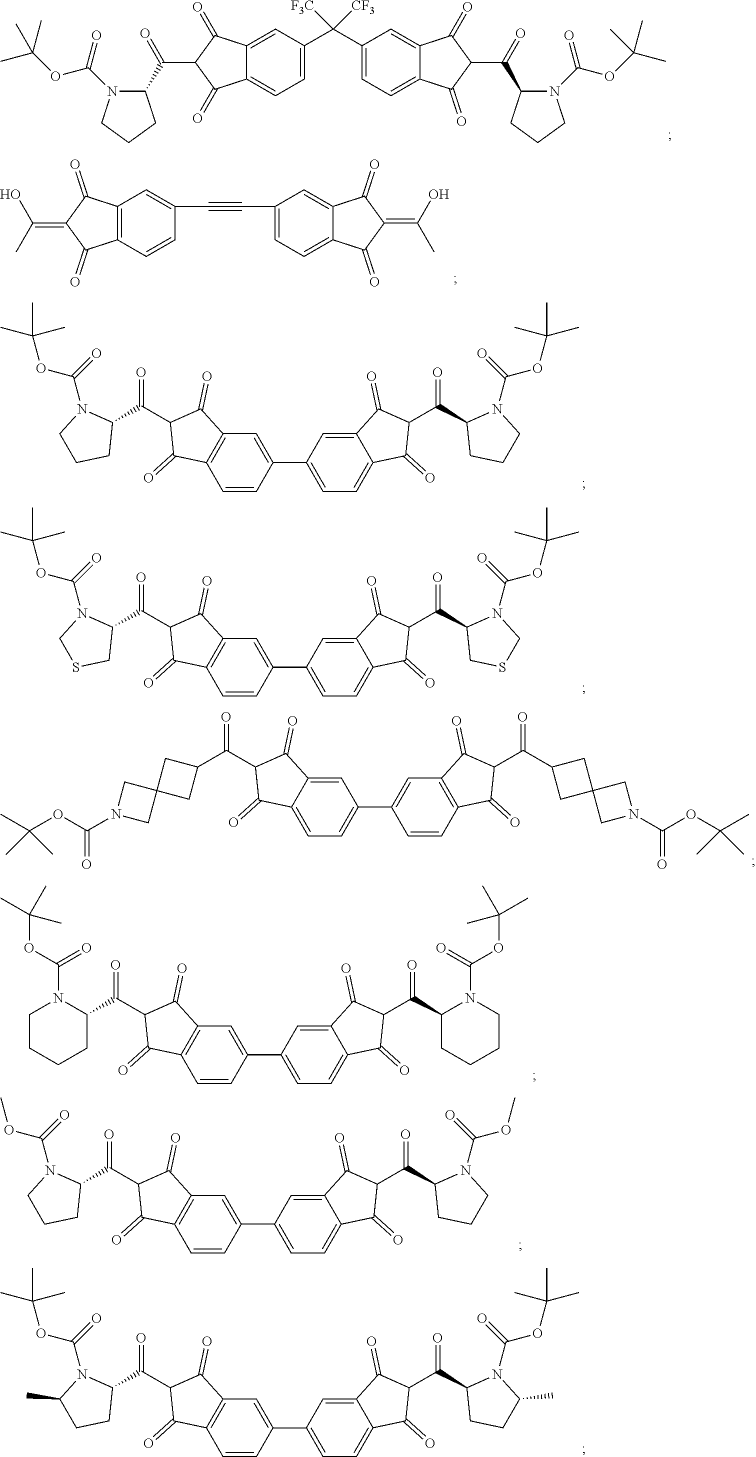 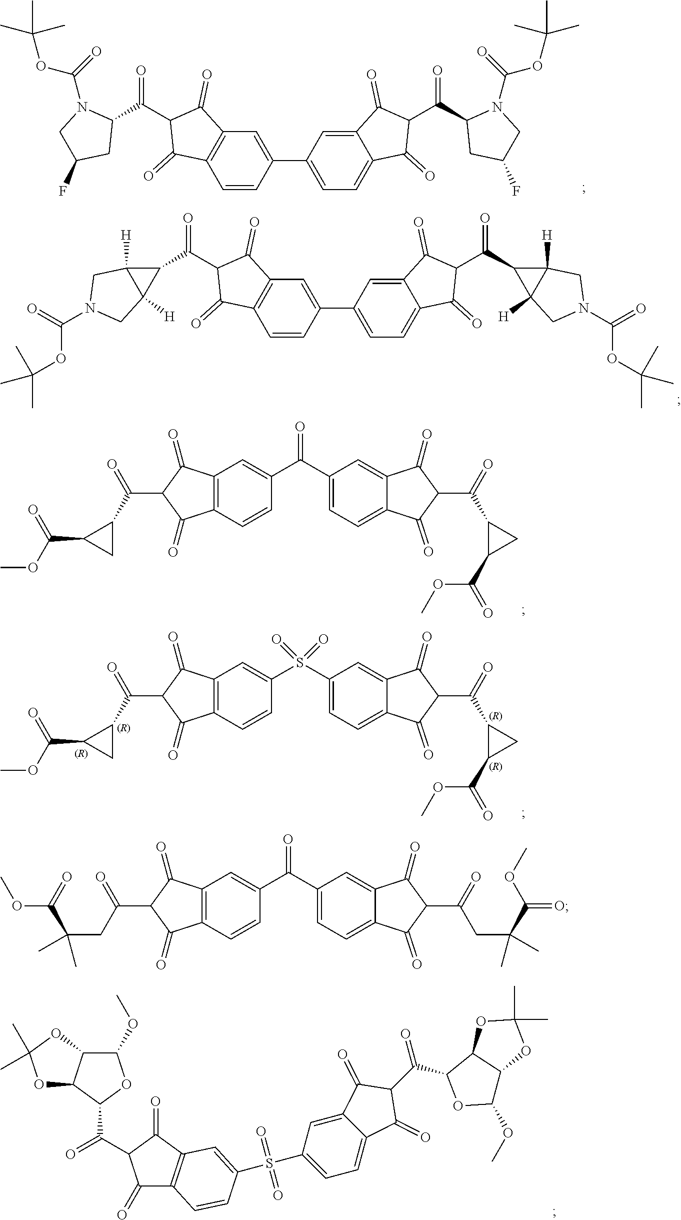 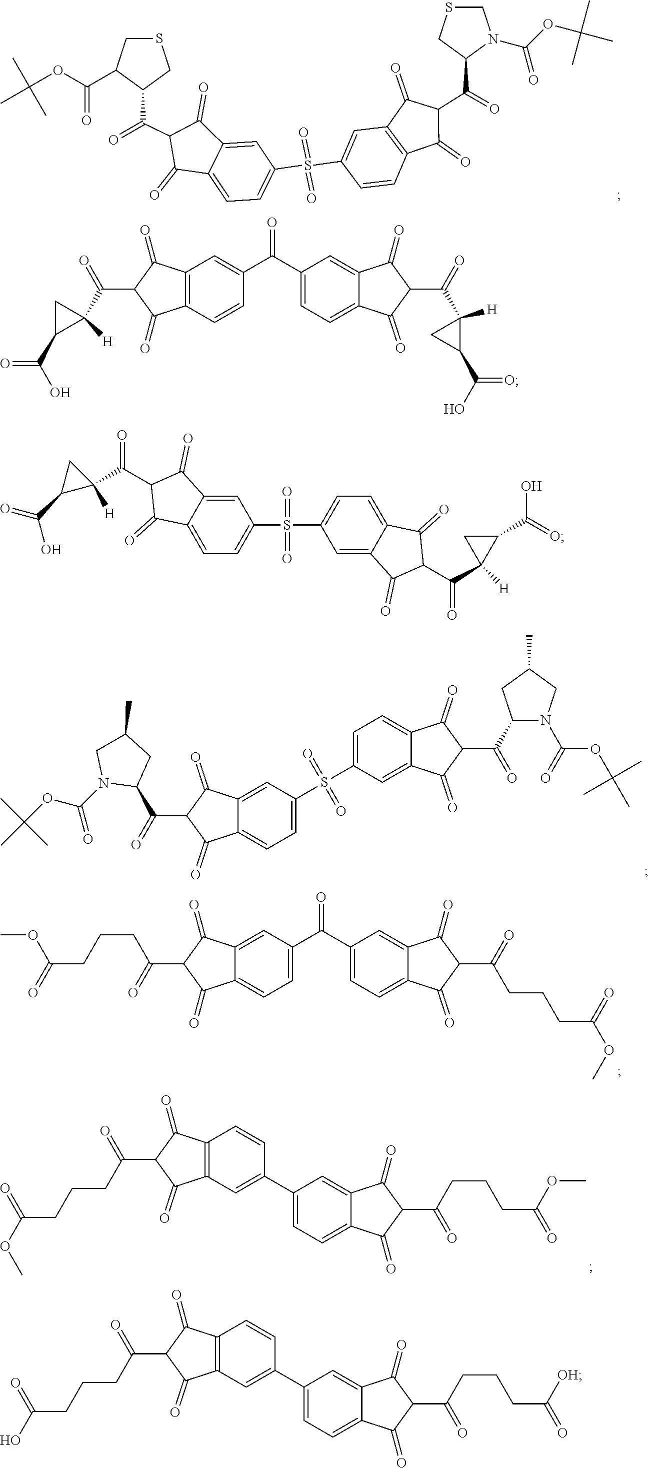 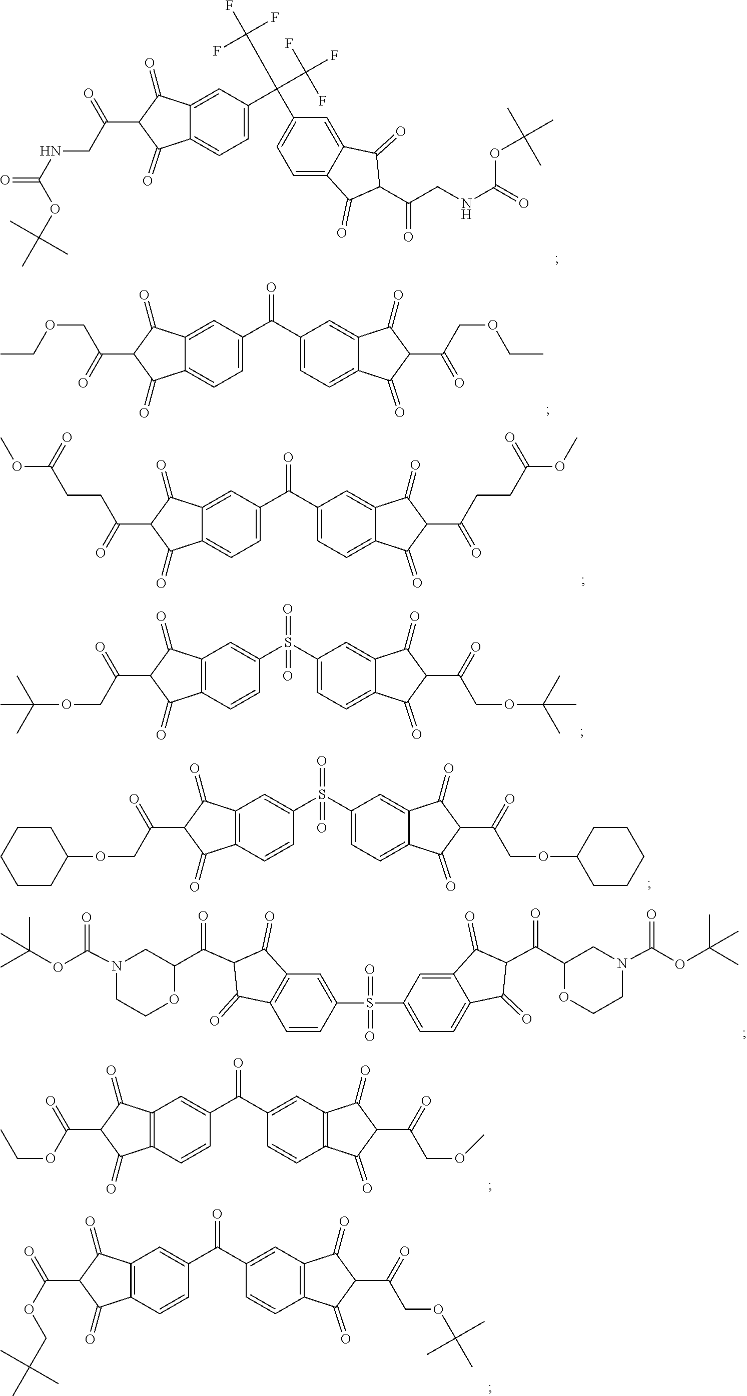 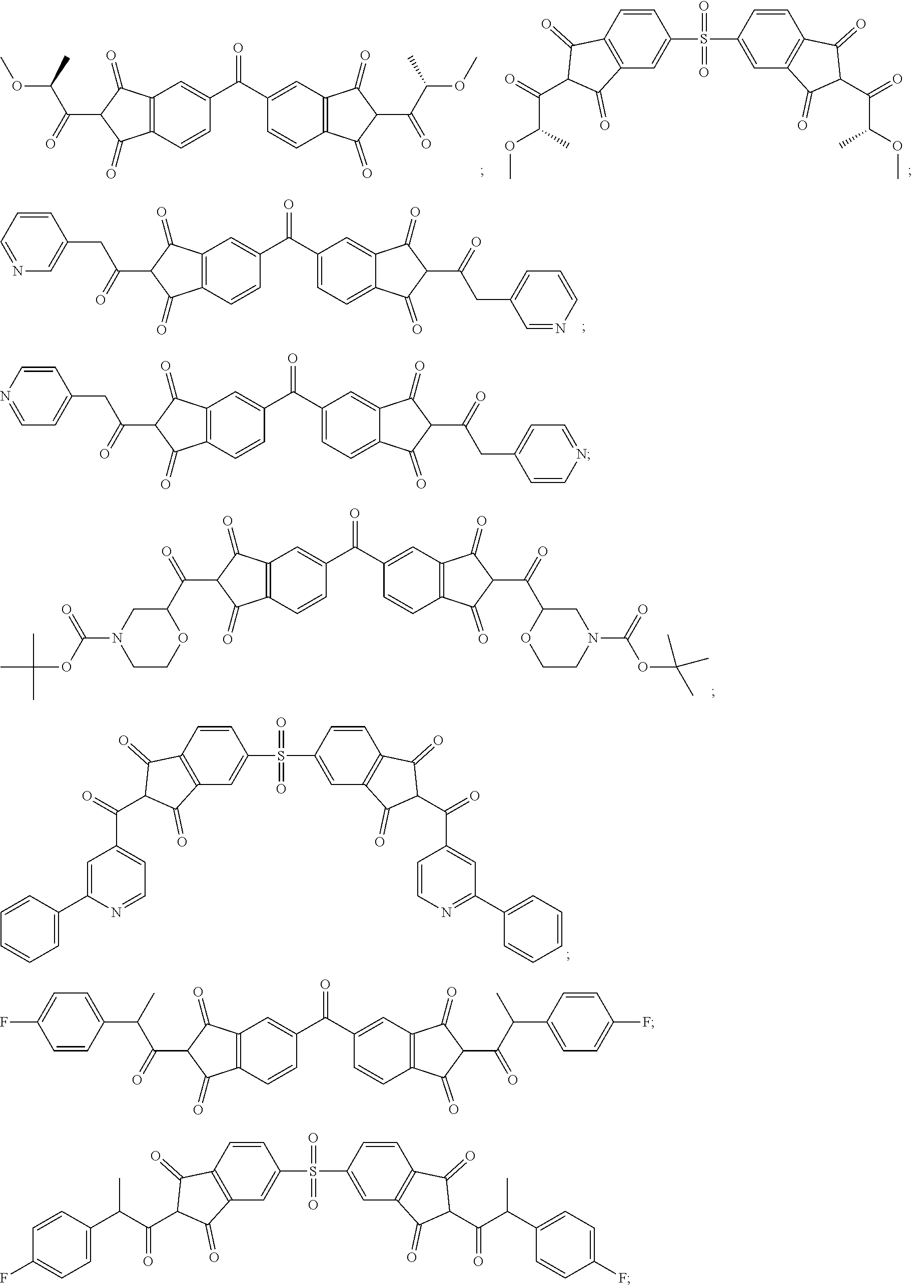 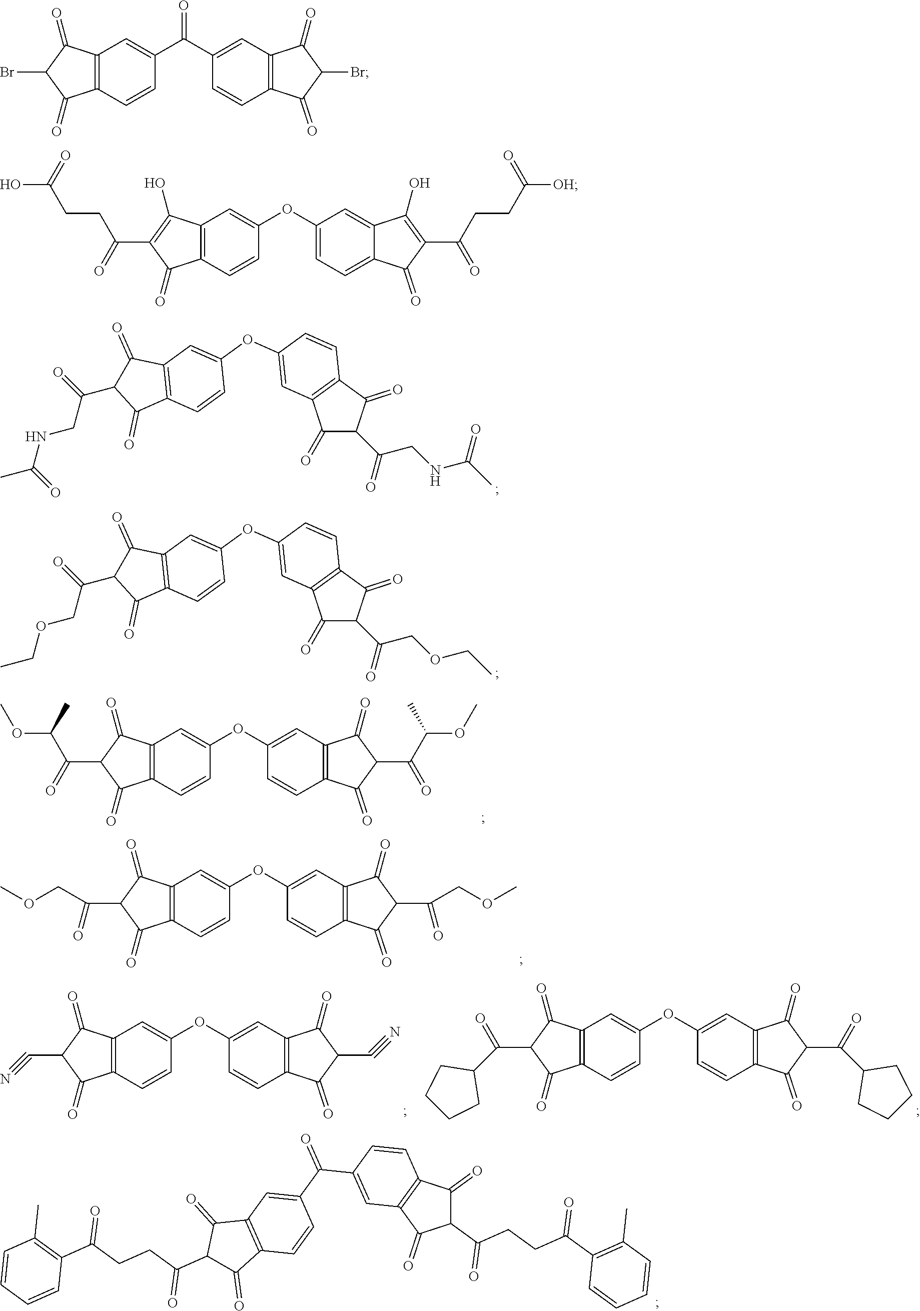 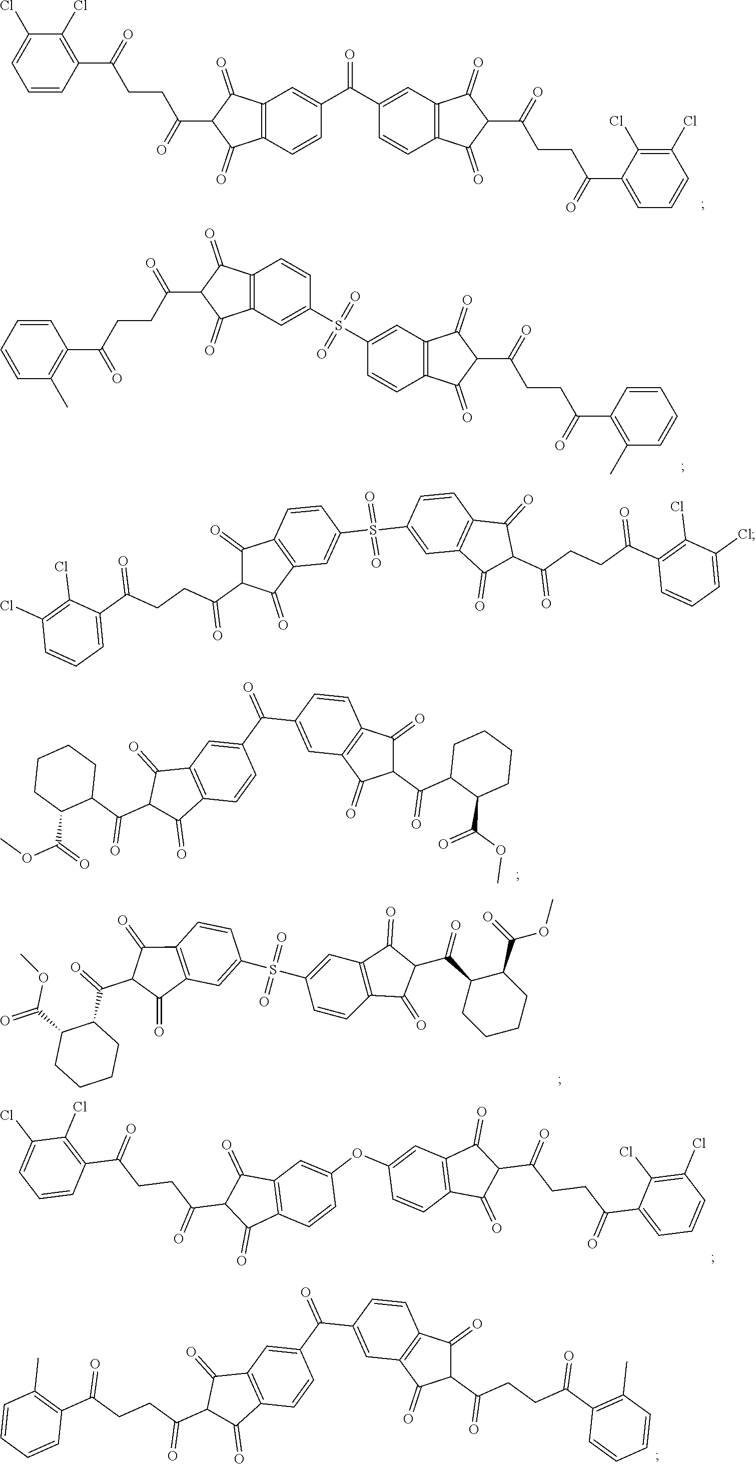 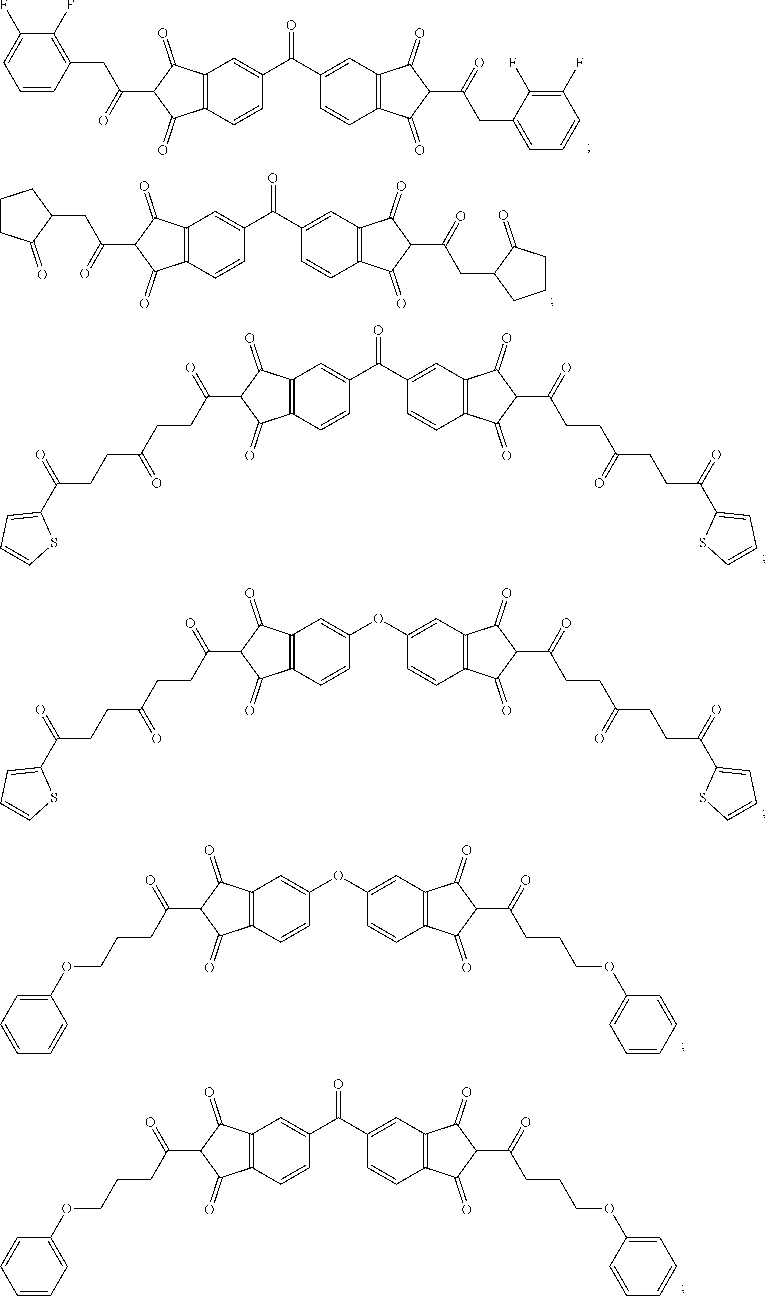 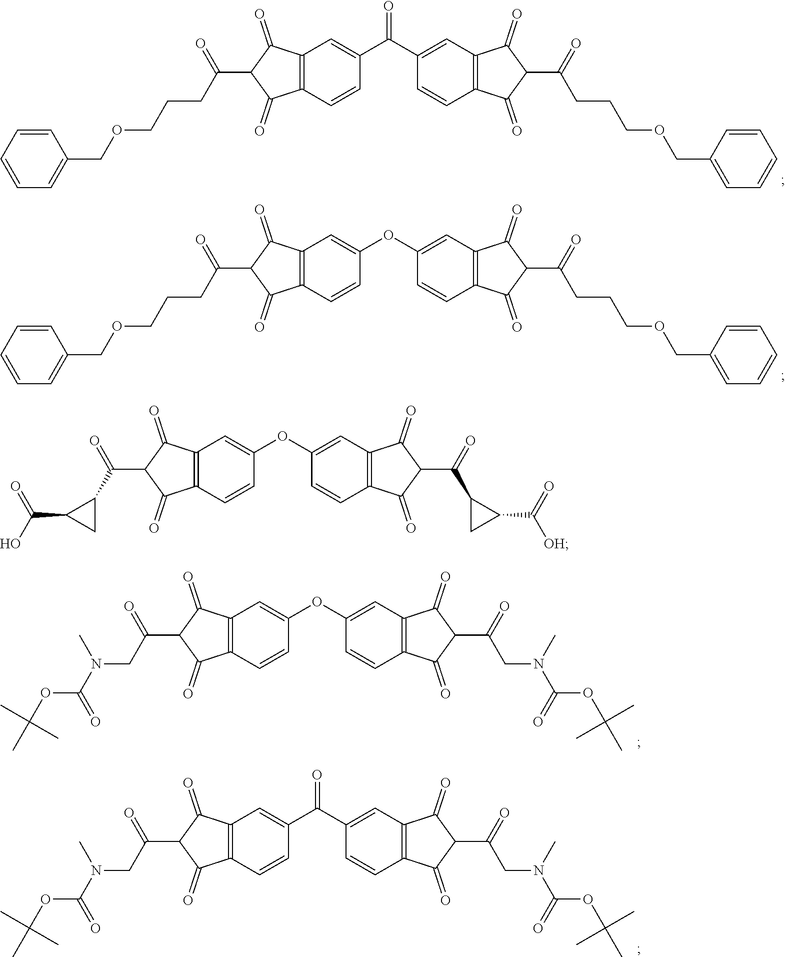 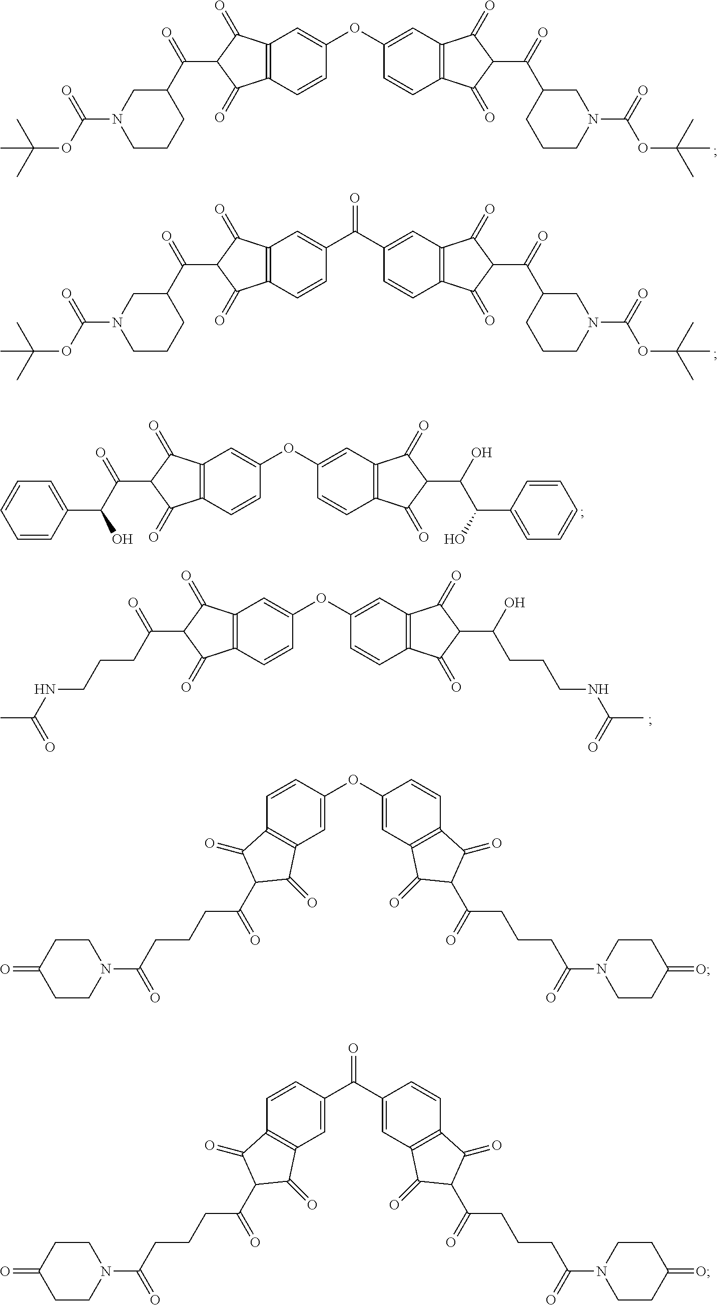 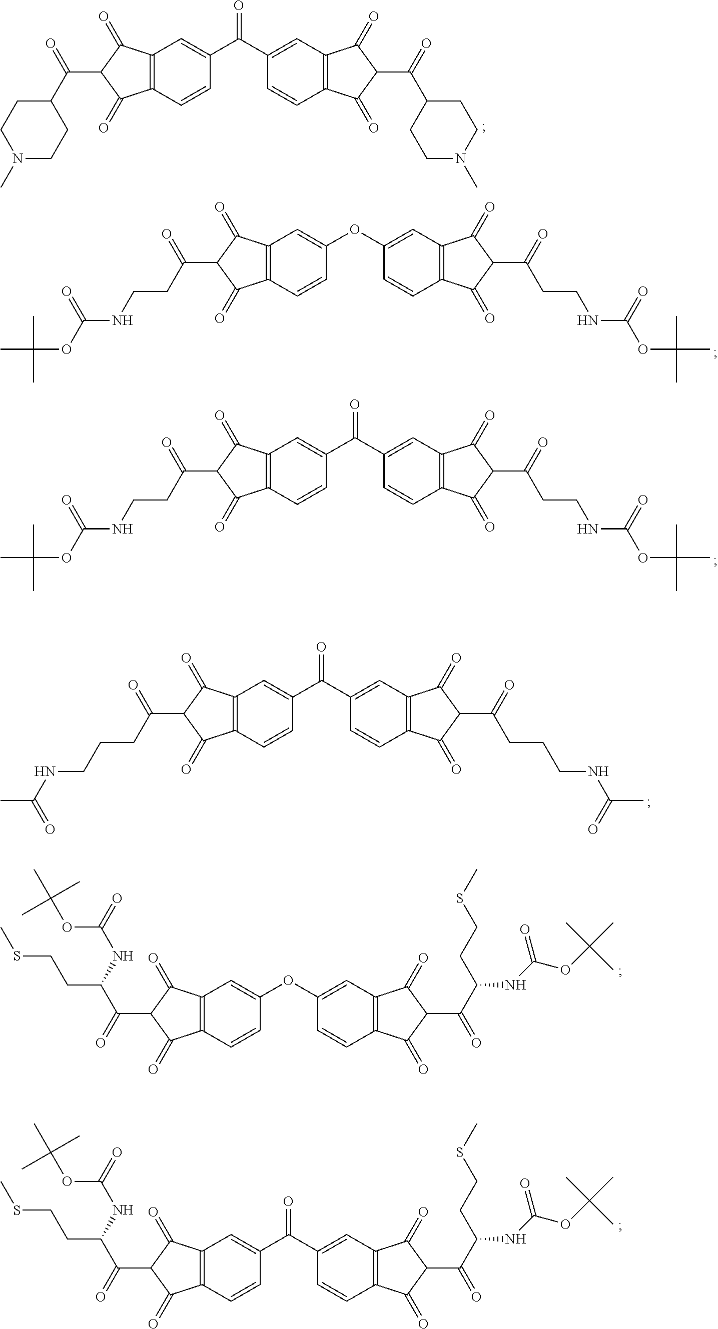 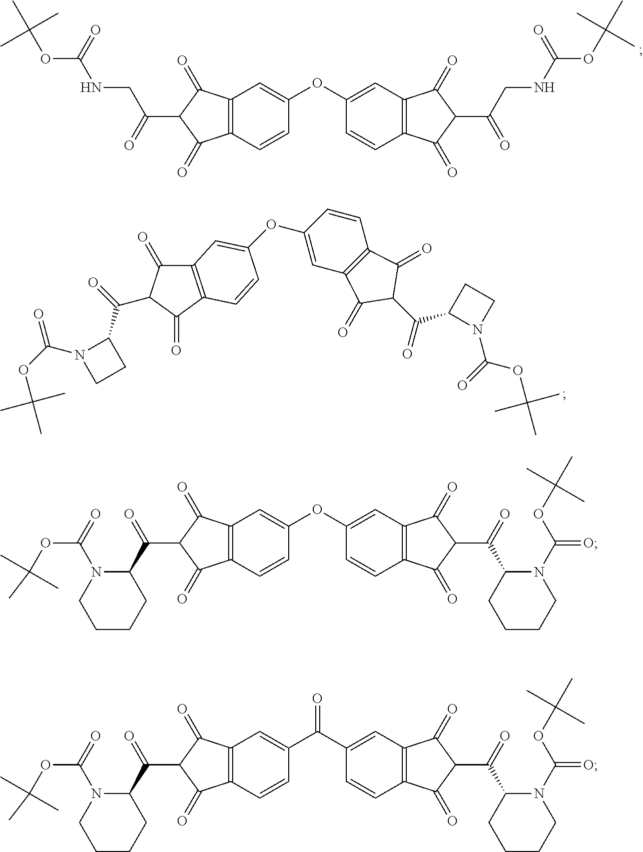 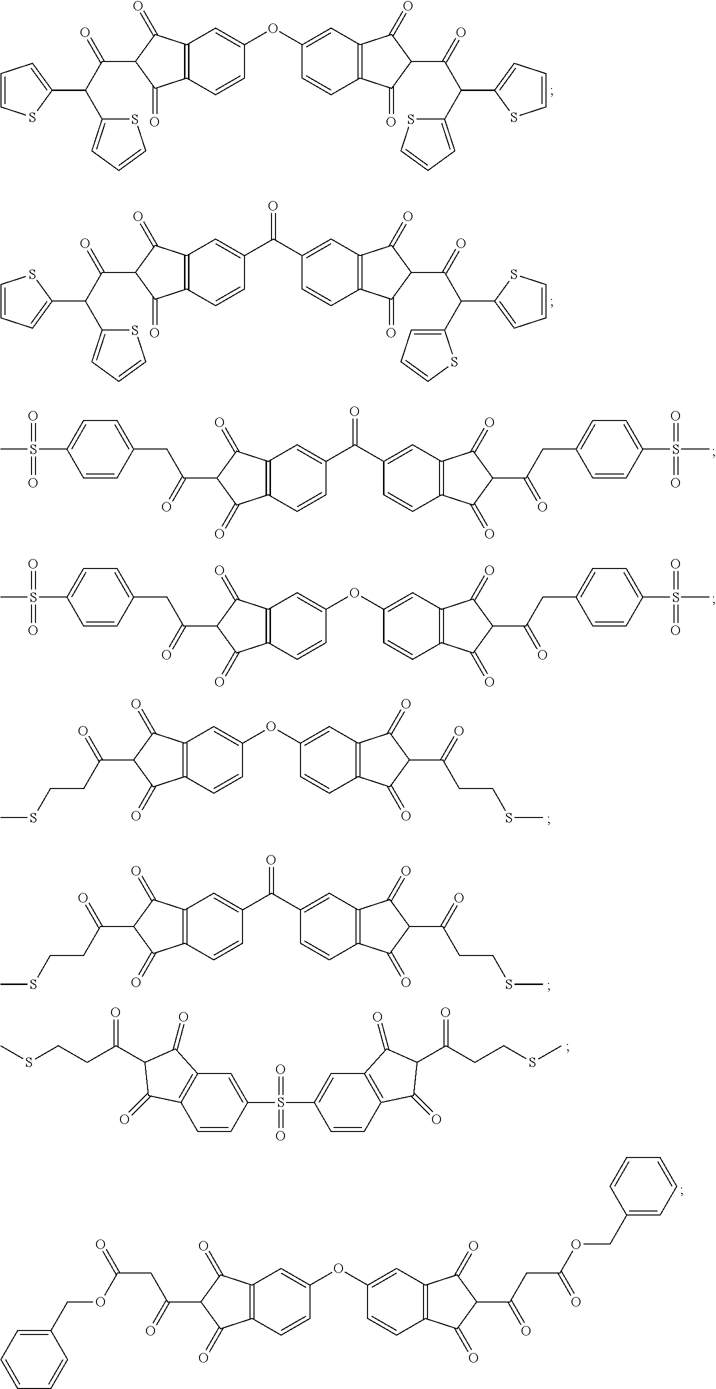 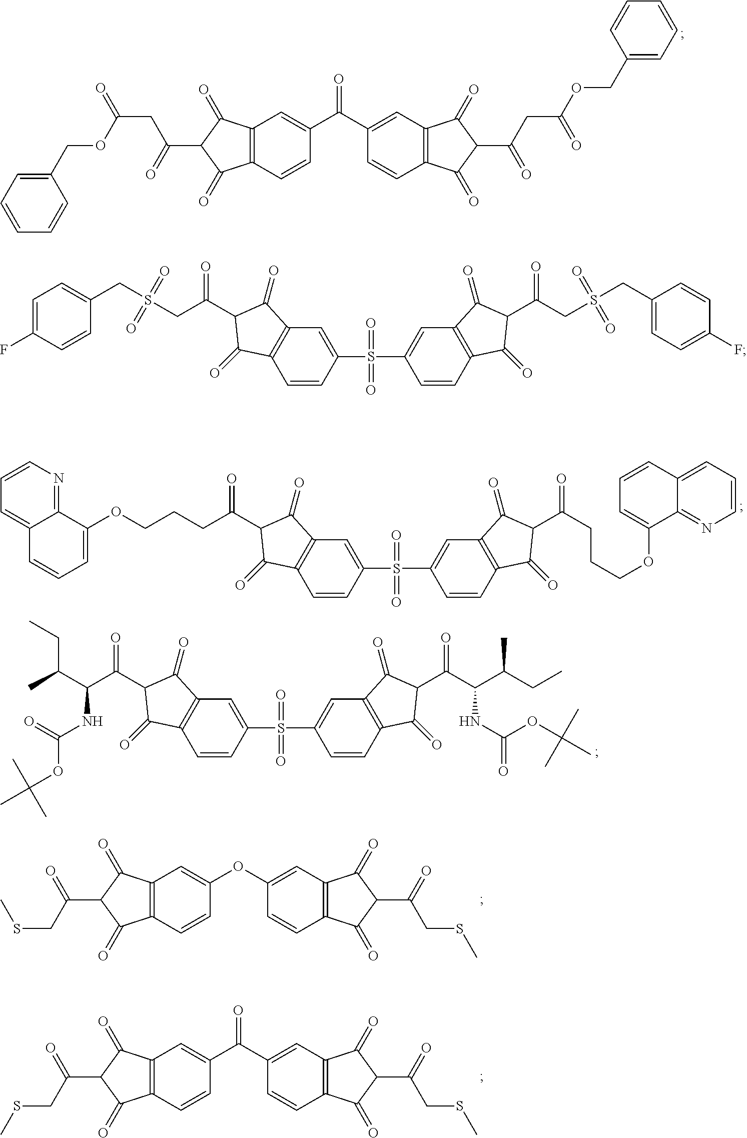 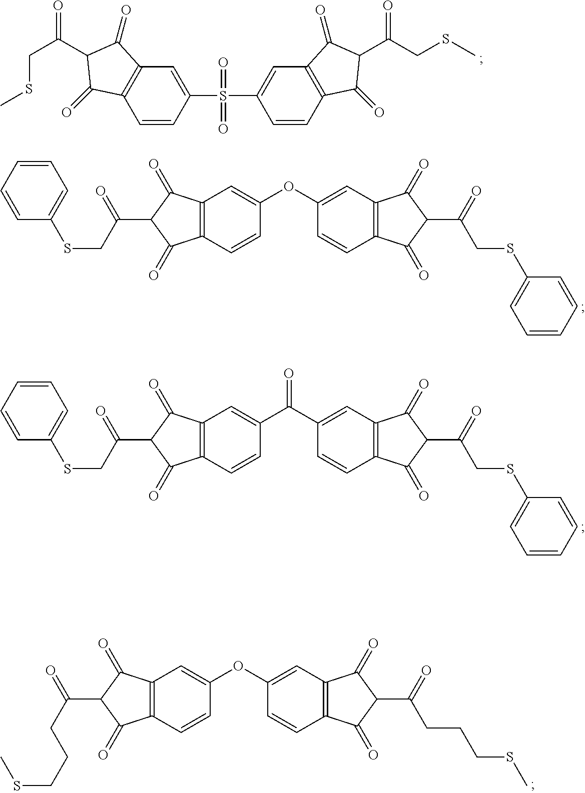 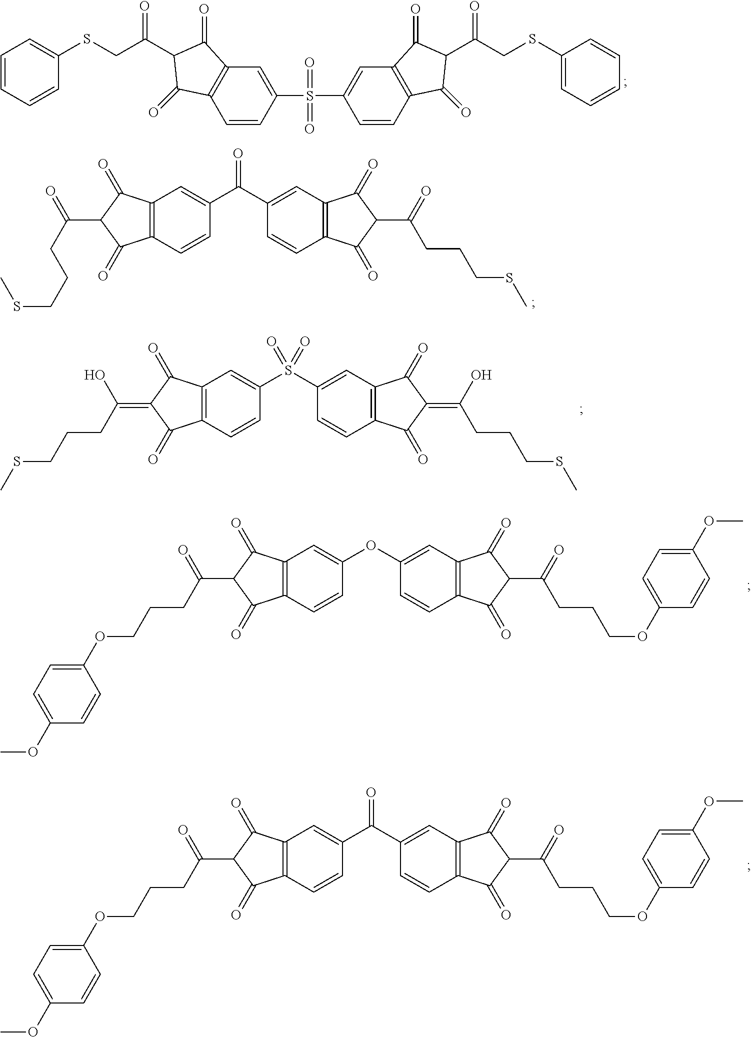 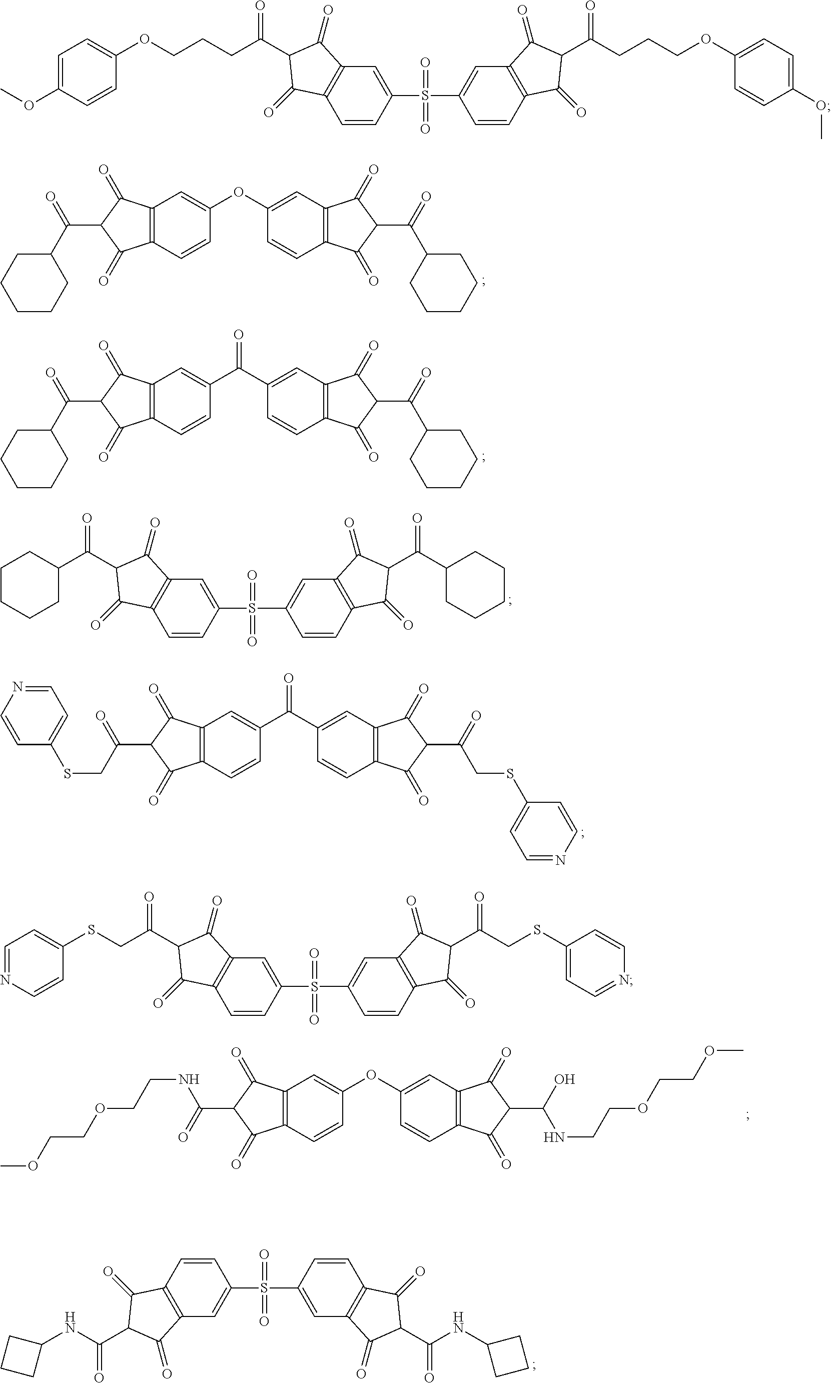 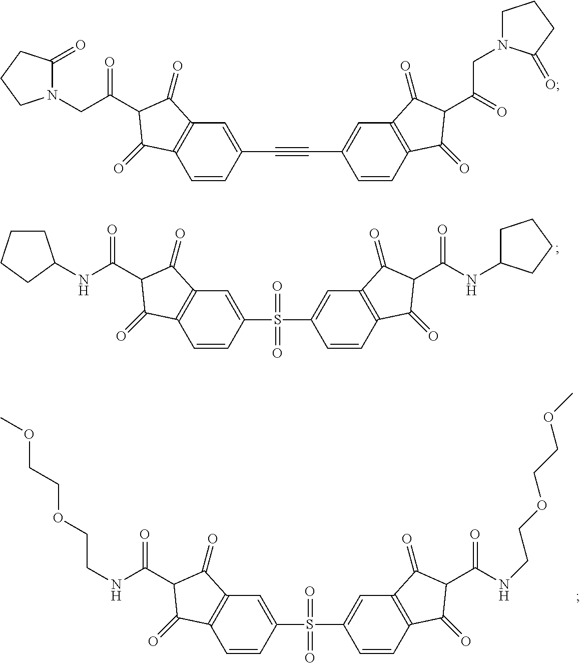 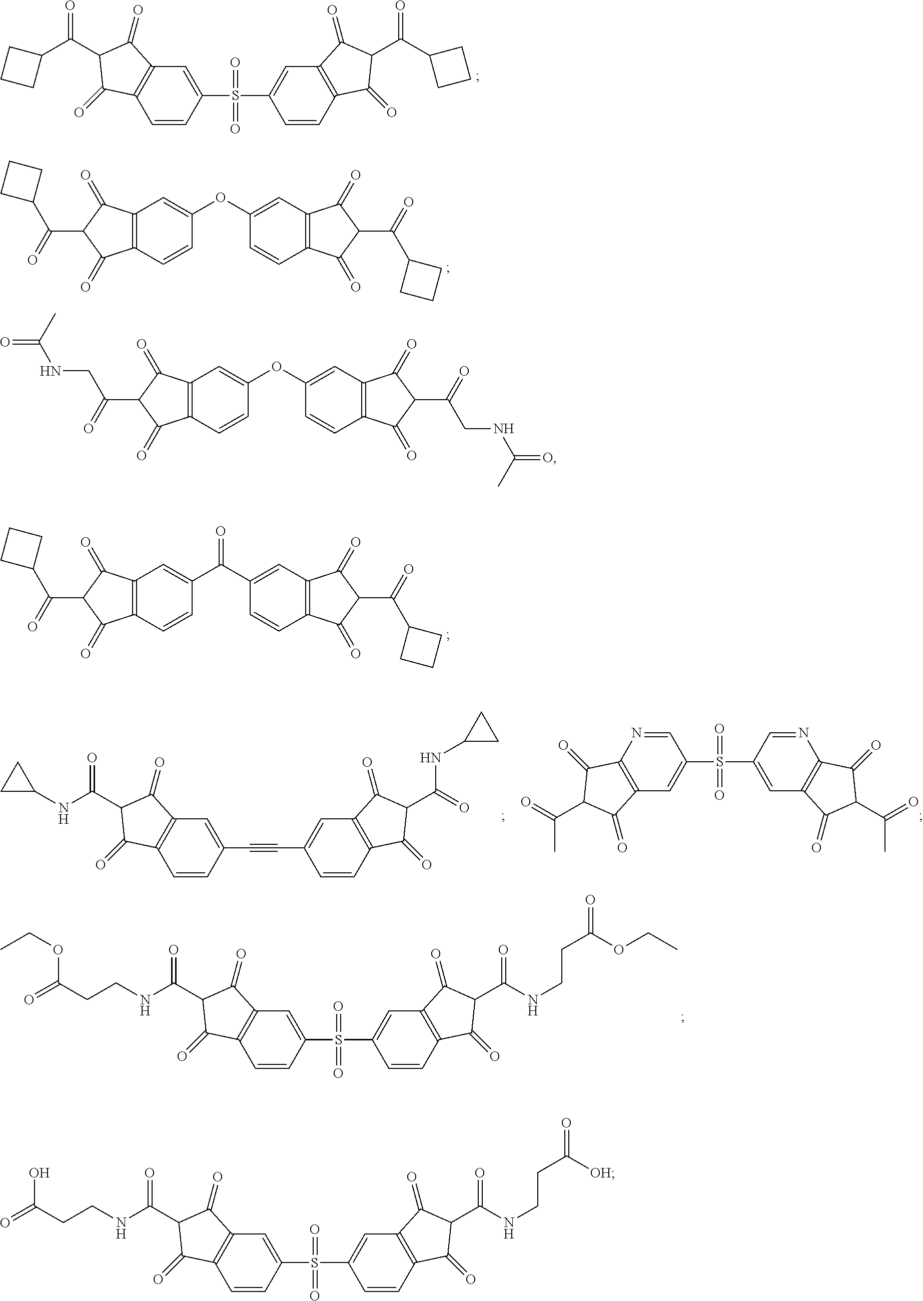 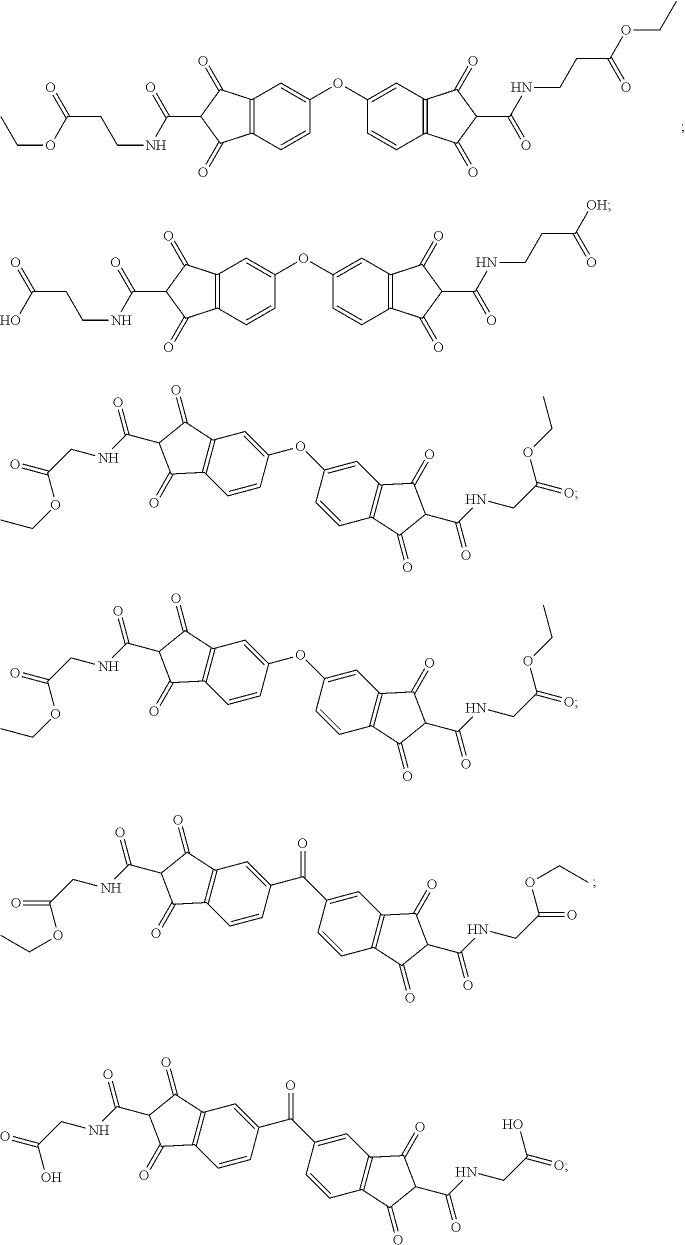 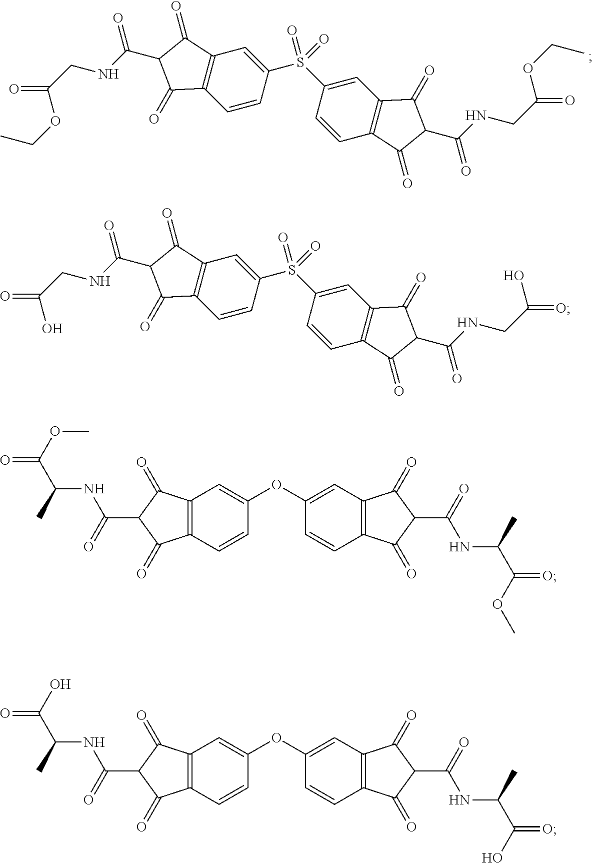 [SMILES: CCOC(=O)CNC(=O)C1C(=O)c2ccc(S(=O)(=O)c3ccc4c(c3)C(=O)C(C(=O)NCC(=O)OCC)C4=O)cc2C1=O ; O=C(O)CNC(=O)C1C(=O)c2ccc(S(=O)(=O)c3ccc4c(c3)C(=O)C(C(=O)NCC(=O)O)C4=O)cc2C1=O ; COC(=O)[C@H](C)NC(=O)C1C(=O)c2ccc(Oc3ccc4c(c3)C(=O)C(C(=O)N[C@@H](C)C(=O)OC)C4=O)cc2C1=O ; C[C@H](NC(=O)C1C(=O)c2ccc(Oc3ccc4c(c3)C(=O)C(C(=O)N[C@@H](C)C(=O)O)C4=O)cc2C1=O)C(=O)O] 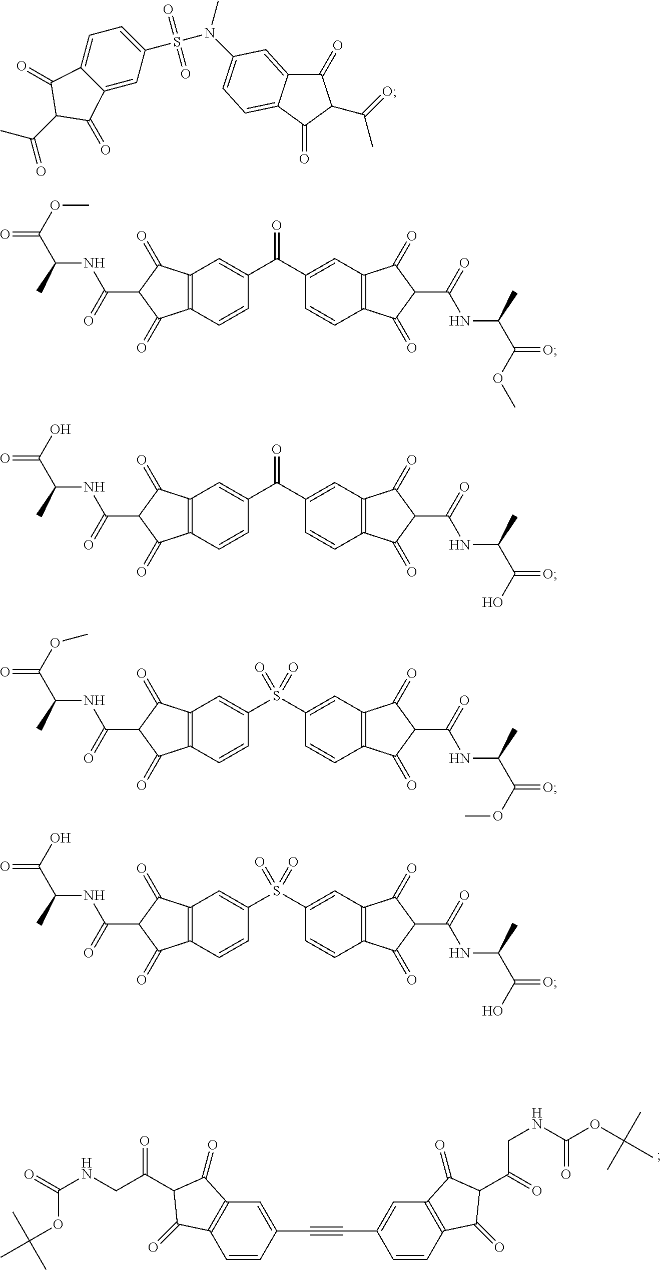 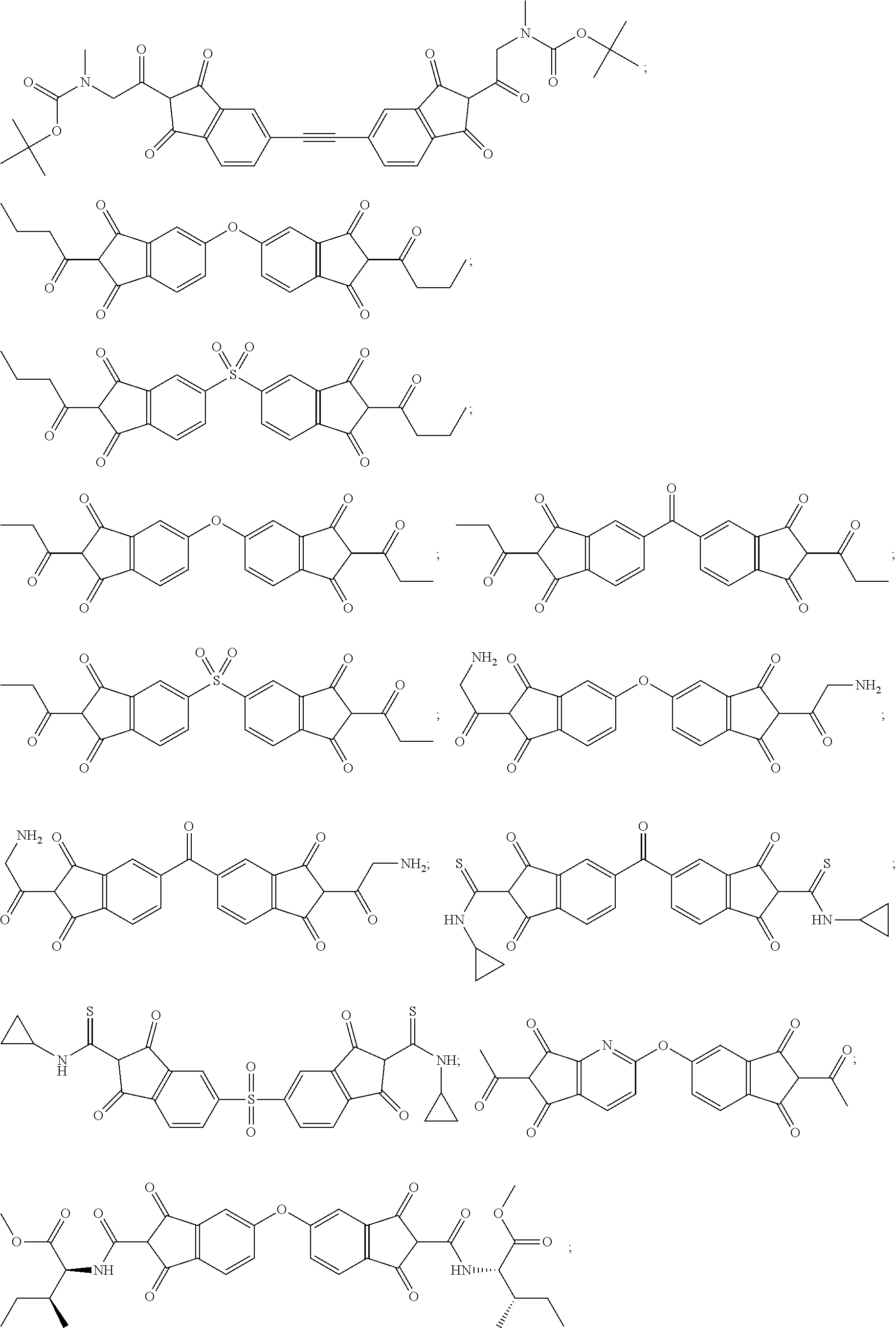 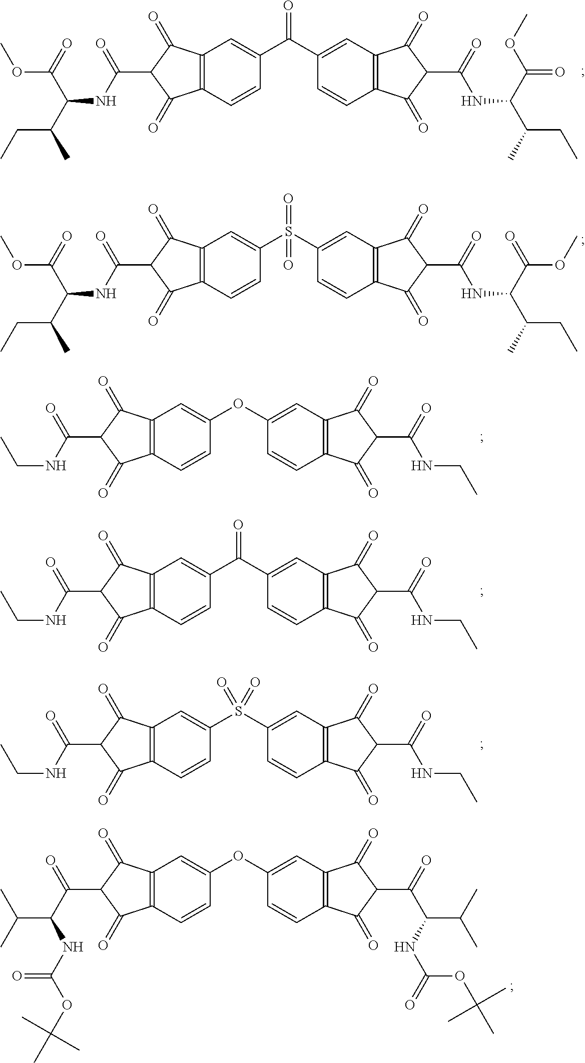 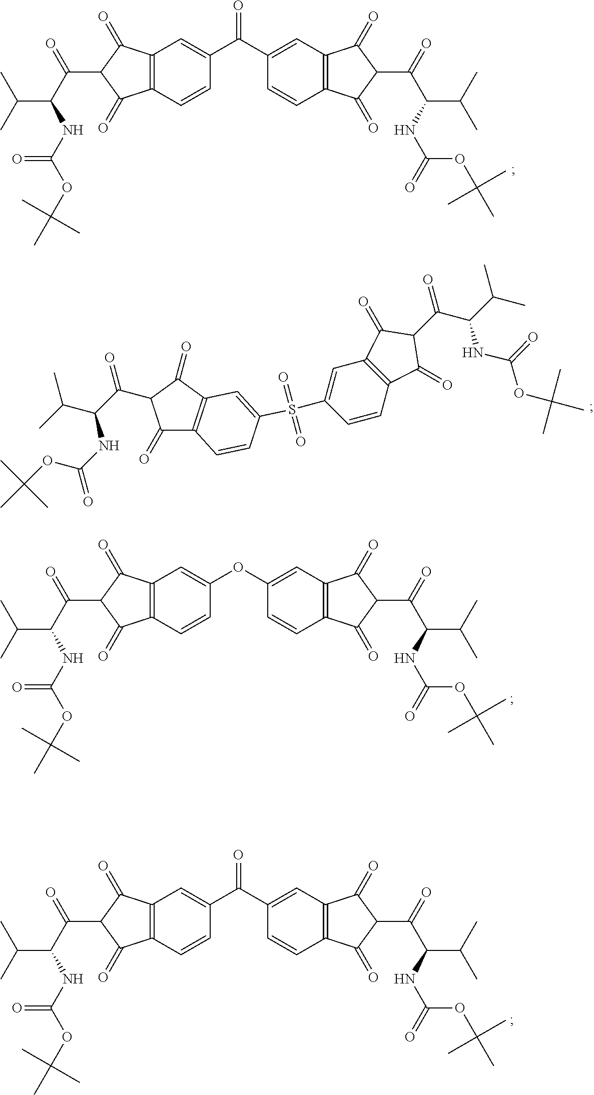 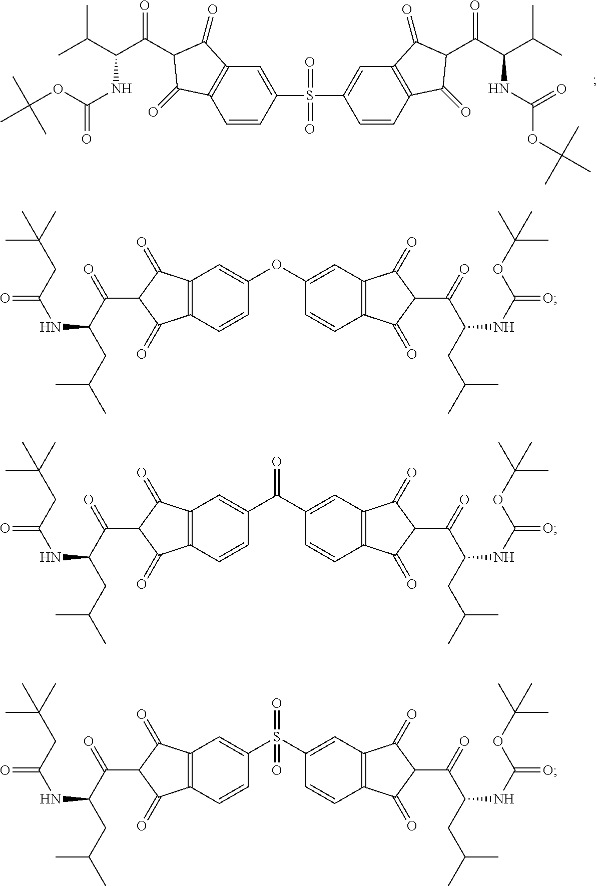 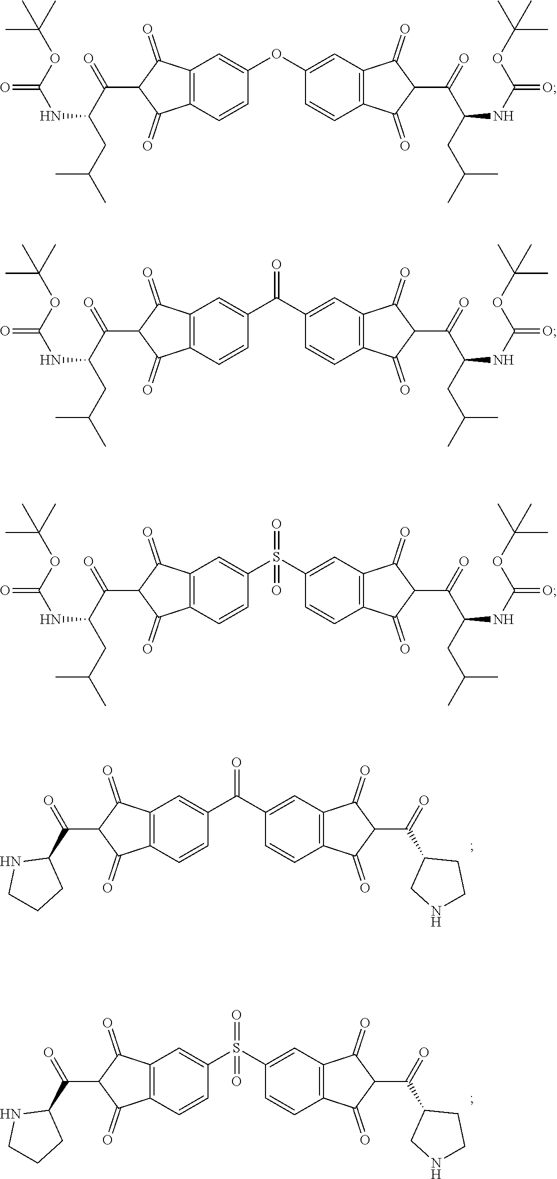 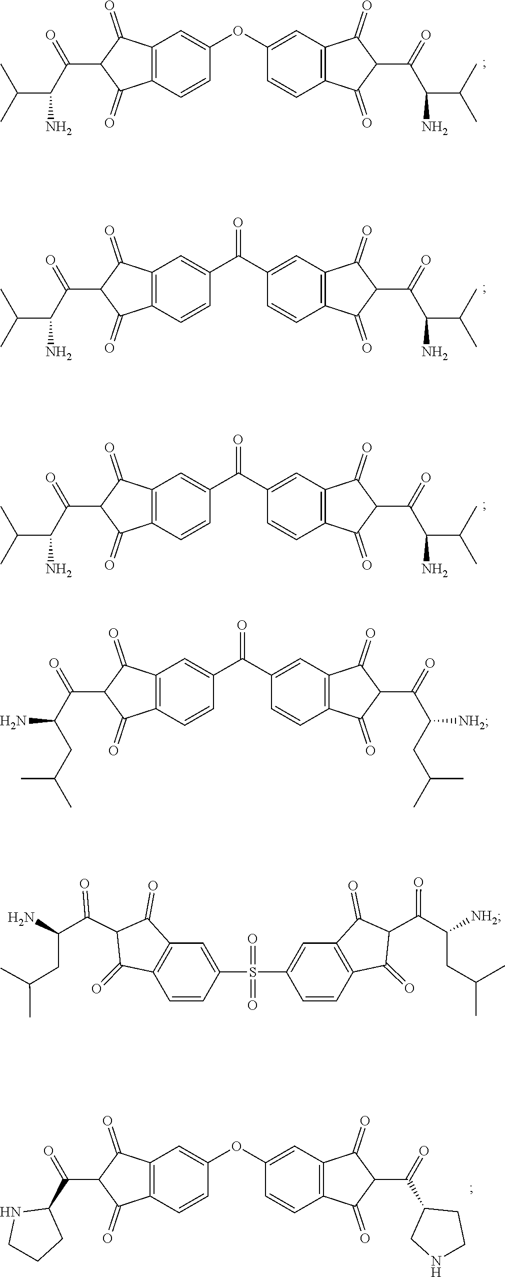 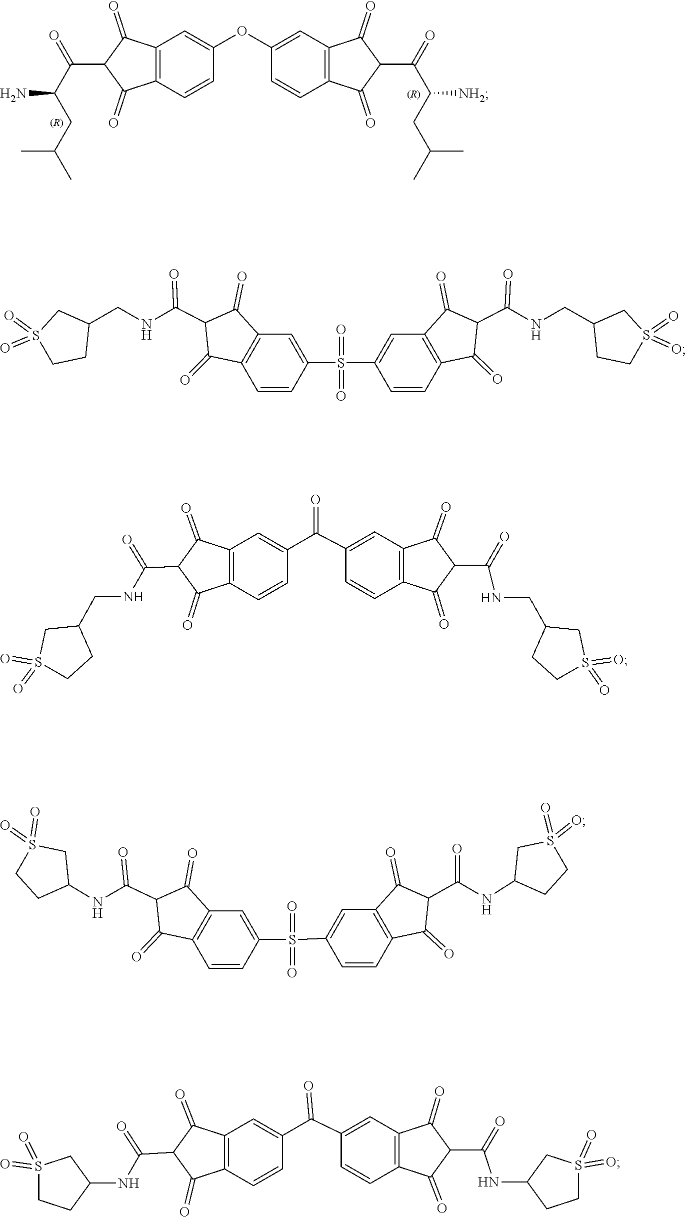 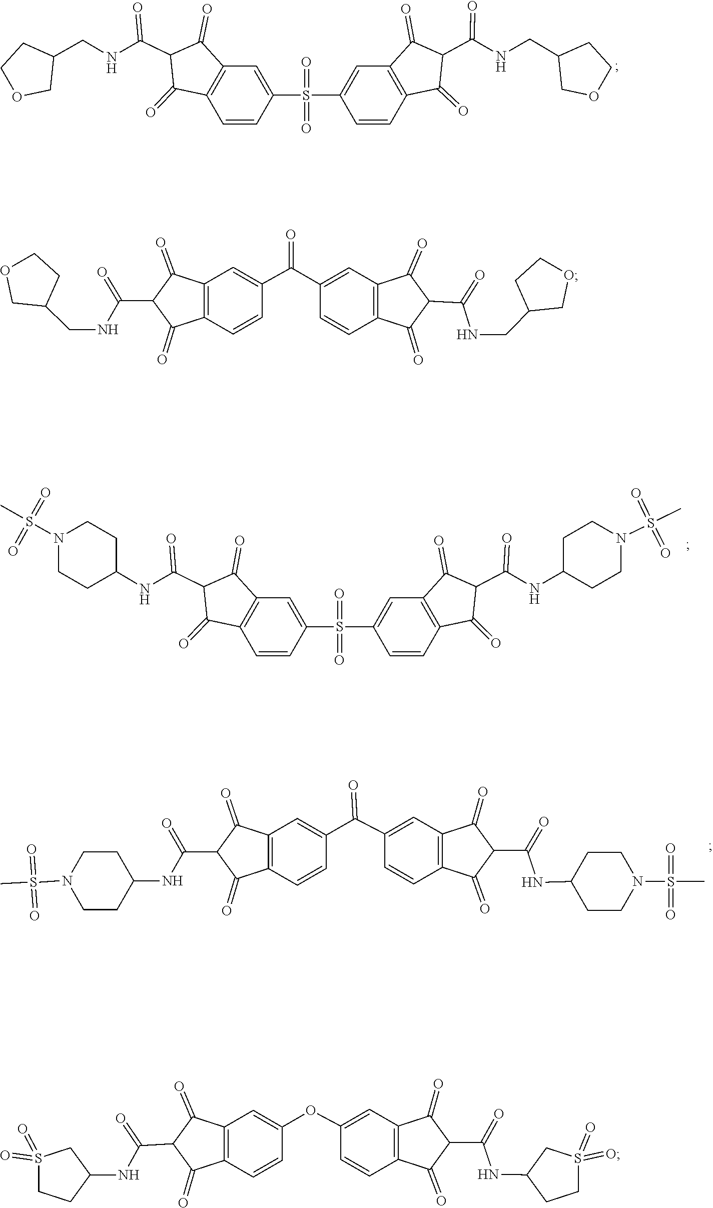 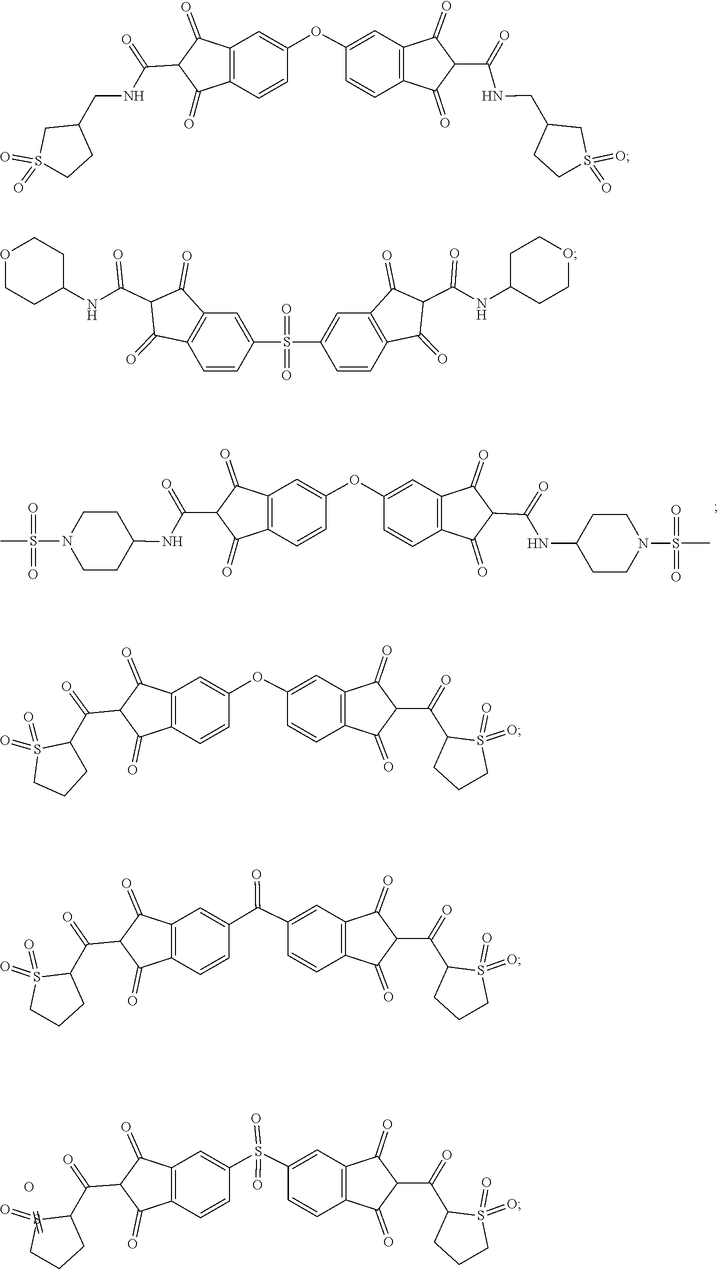 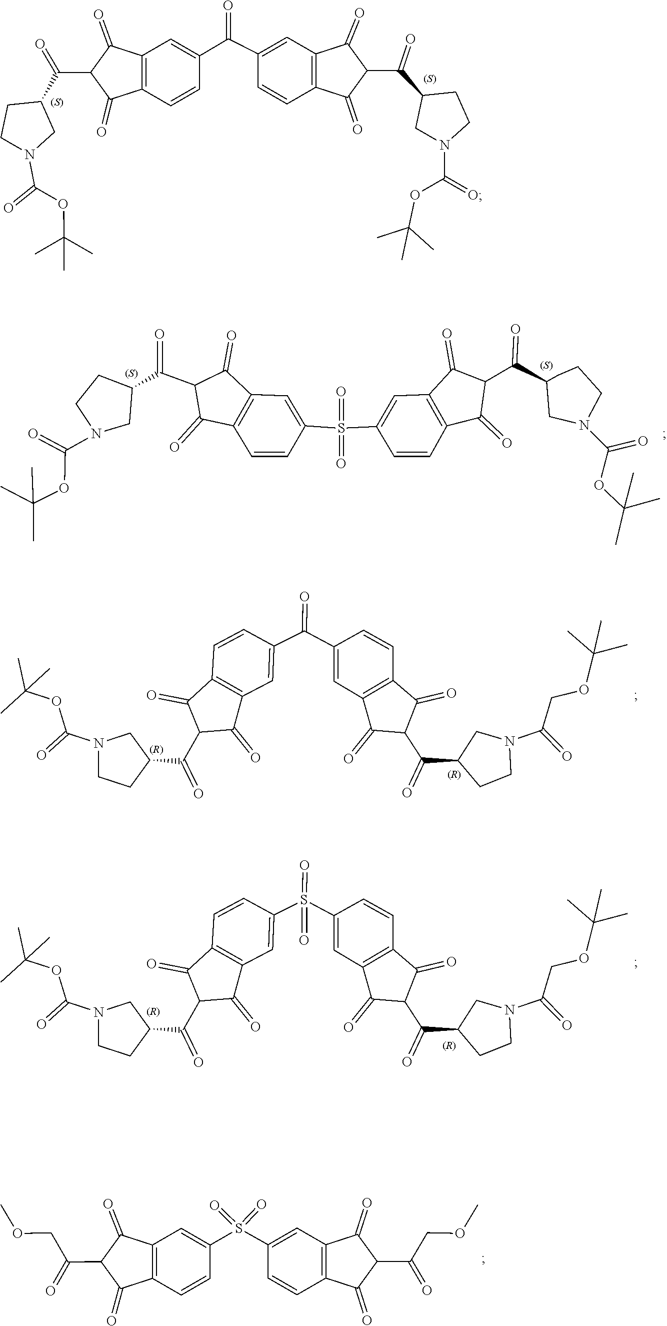 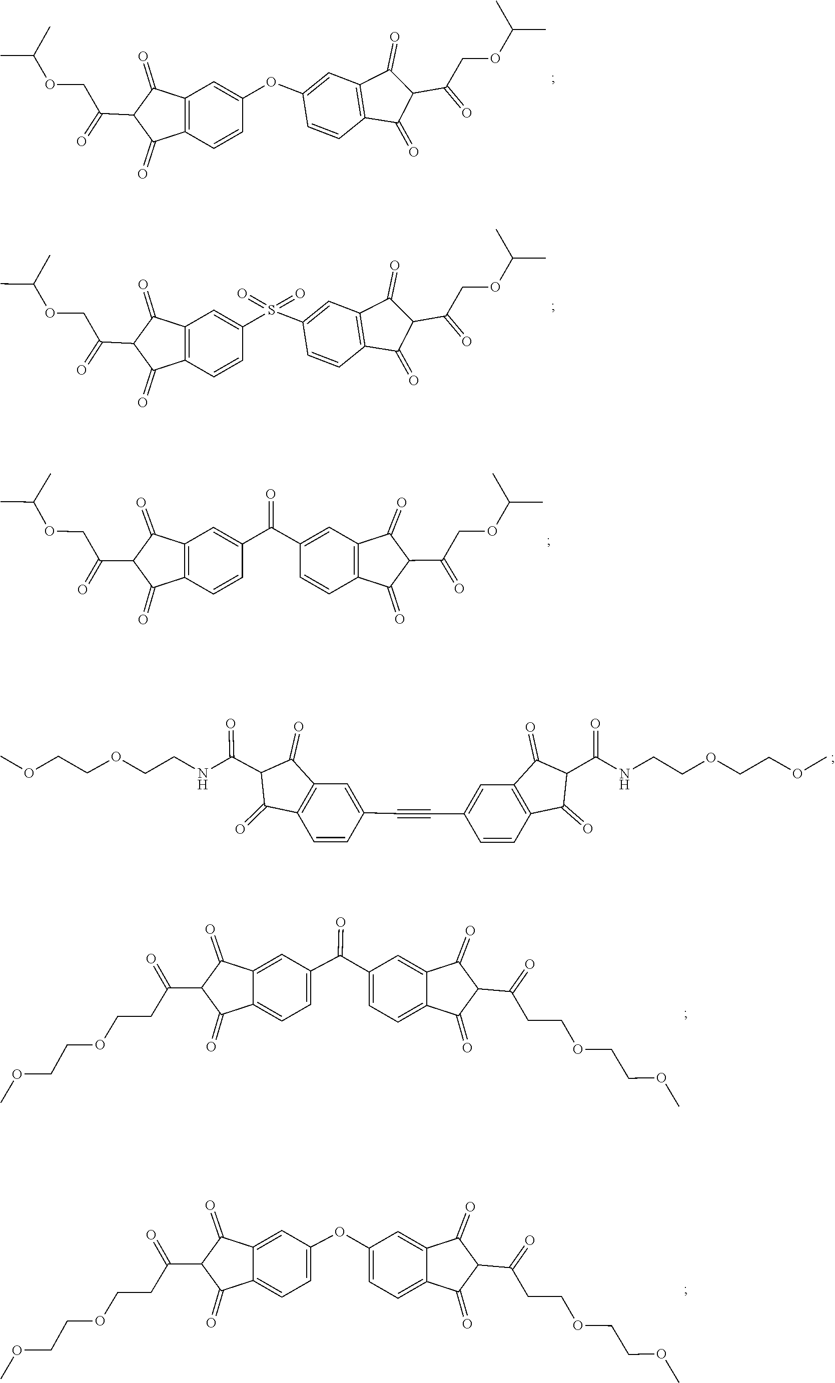 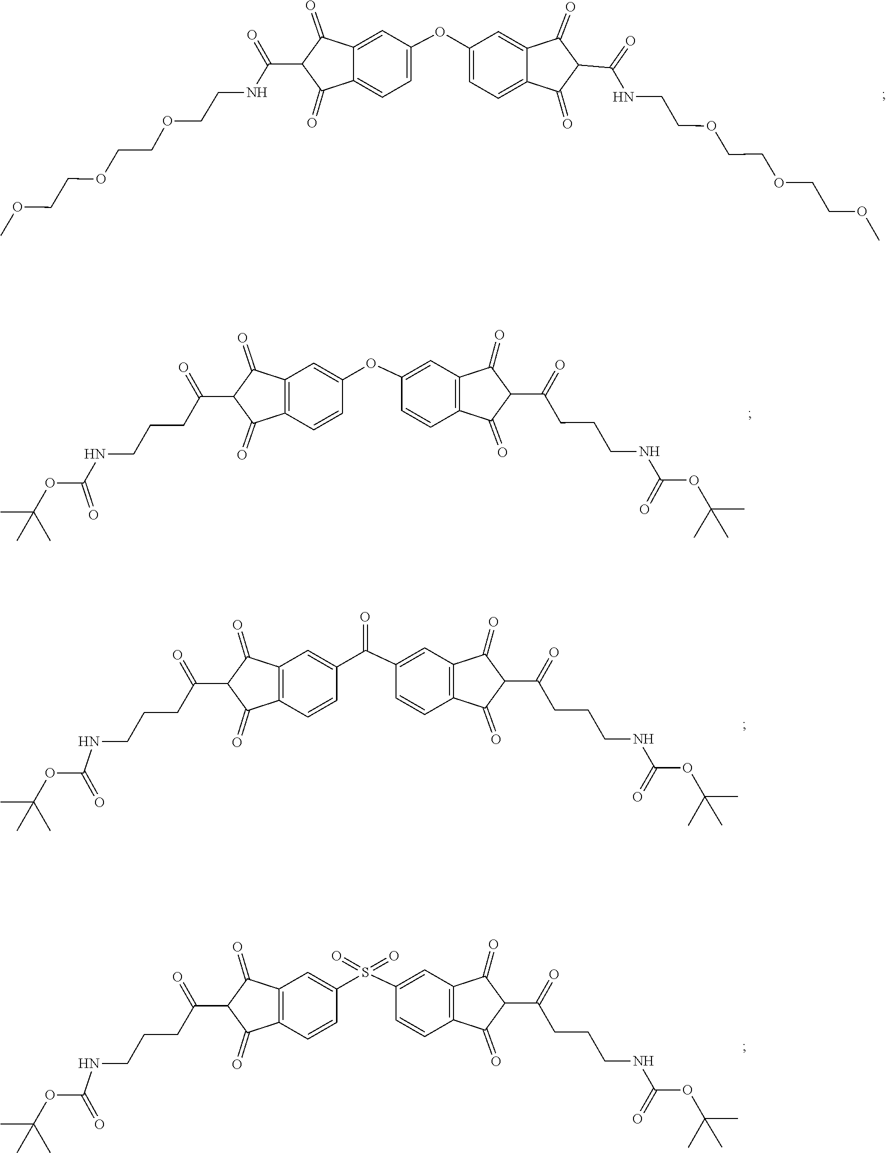 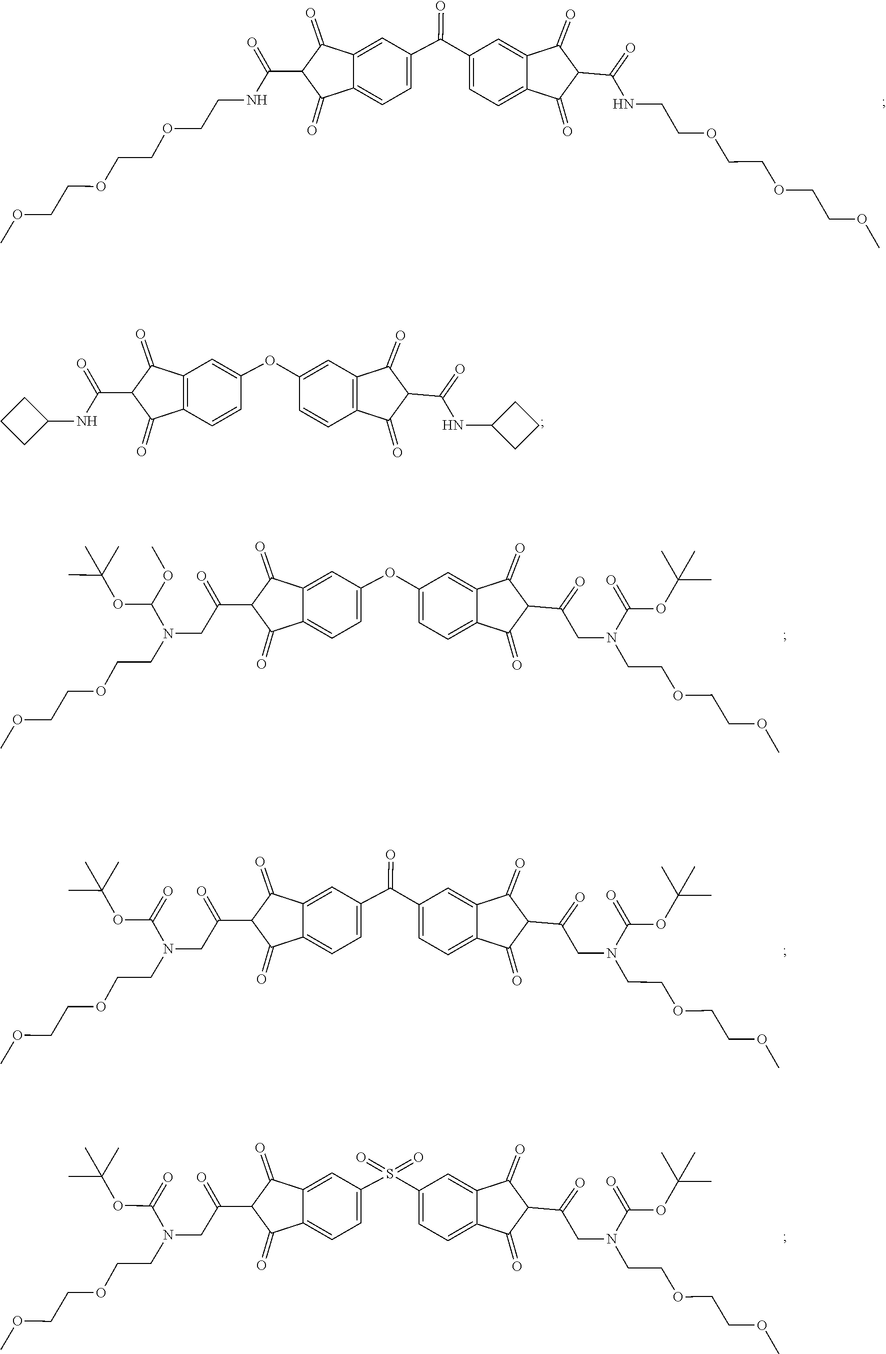 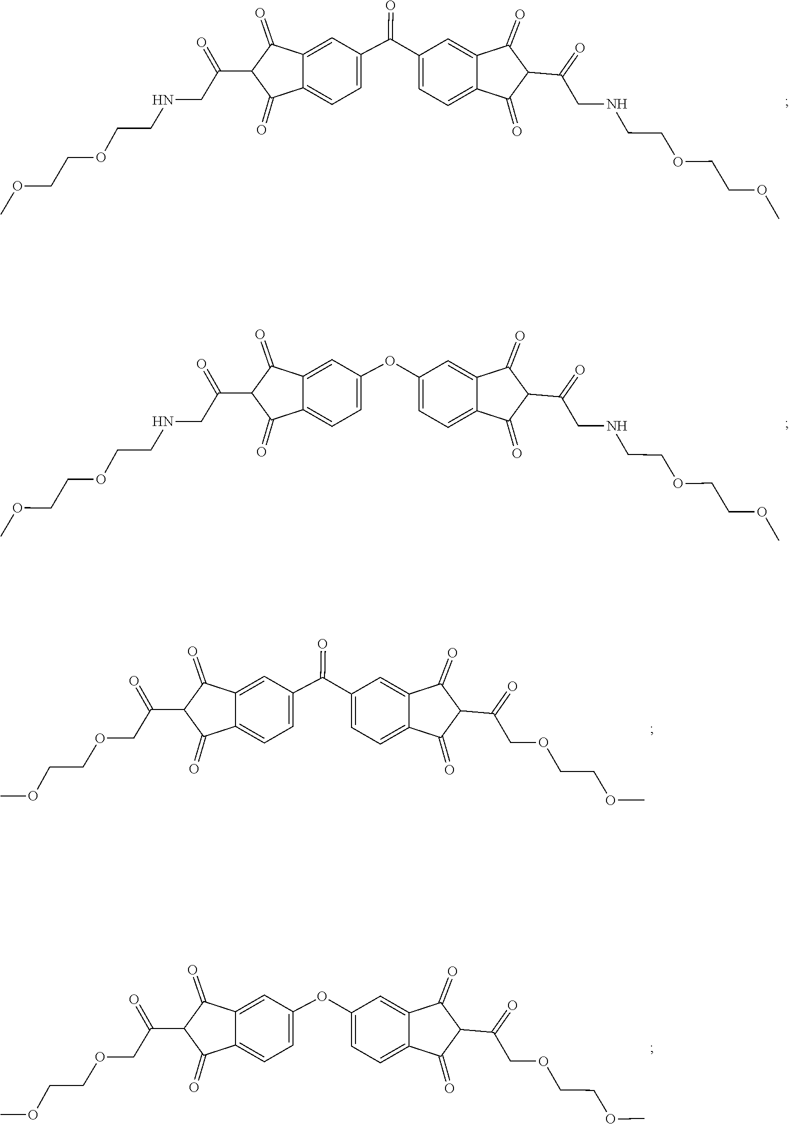 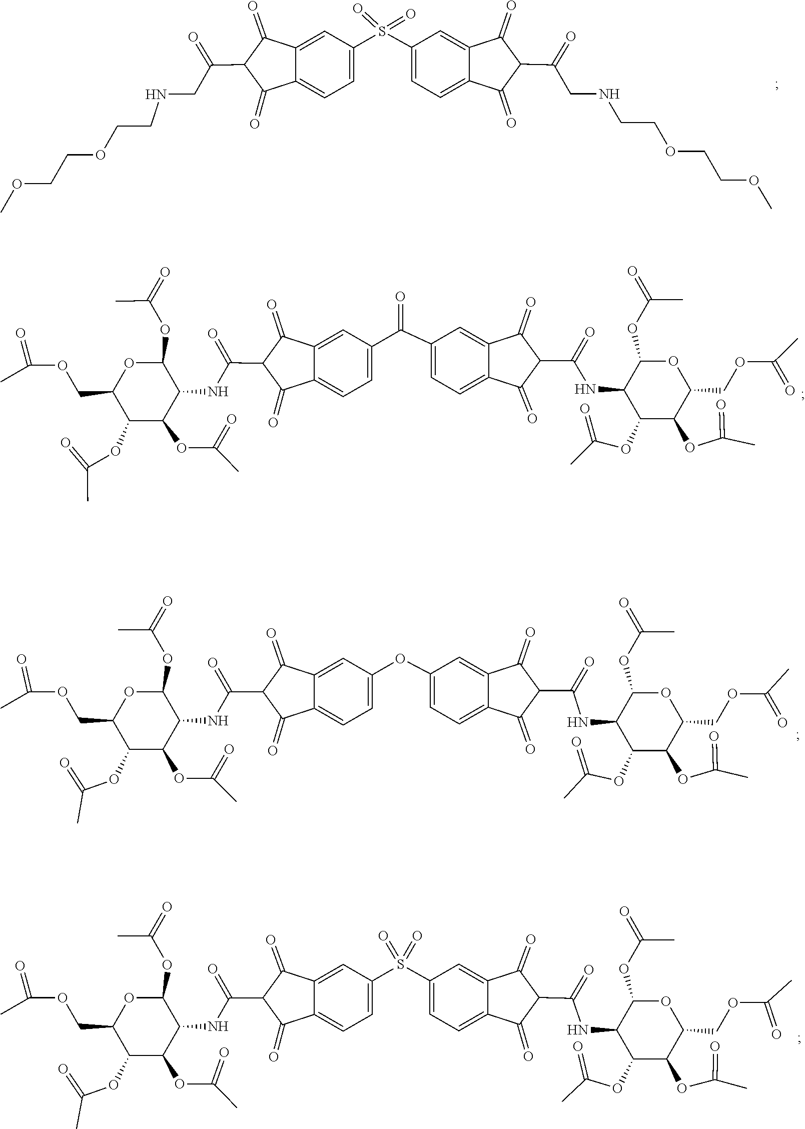 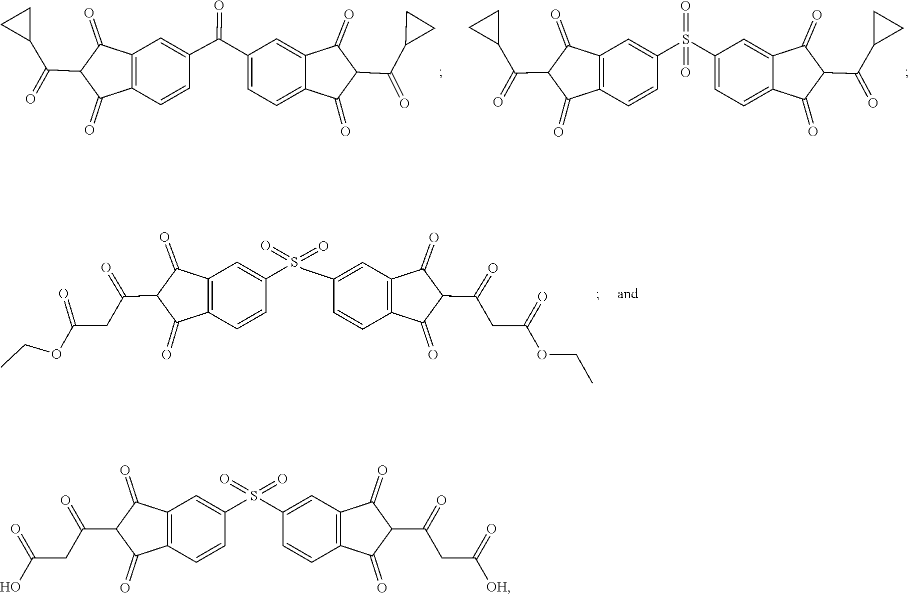
or a pharmaceutically acceptable salt thereof.

67. A pharmaceutical composition comprising a compound of claim 1, or a pharmaceutically acceptable salt thereof, and a pharmaceutically acceptable carrier.

68. A method of:
preventing formation of platelet factor-4 (PF4) tetramers in a subject; disrupting platelet factor-4 (PF4) tetramers in a subject; preventing formation of an ultra-large complex (ULC) comprising a PF4 tetramer and a glycosaminoglycan (GAG) or other polyanion in a subject; inhibiting ULC-antibody complex binding to a FcγRIIa receptor on a platelet in a subject; inhibiting platelet aggregation in a subject, increasing high density lipoproteins in a subject; modulating clotting or hemostasis in a subject; correcting a platelet imbalance in a subject; or a combination thereof,
the method comprising administering to the subject a therapeutically effective amount of the compound of claim 1, or a pharmaceutically acceptable salt thereof, or a pharmaceutical composition comprising the compound.

69. The method of claim 68, wherein the compound of binds to a PF4 monomer, PF4 dimer, or PF4 tetramer.

70. The method of claim 68, wherein the compound of any one of claims 1-66 disrupts a salt bridge between two PF4 dimers, two or more PF4 monomers, or a PF4 dimer and a PF4 monomer, in a PF4 tetramer.

71.-74. (canceled)

75. The method of claim 68, wherein the GAG is a heparin.

76. The method of claim 68, wherein the platelet imbalance results from heparin administration to said subject.

77.-79. (canceled)

80. A method of treating or preventing a disease or condition selected from:
heparin induced thrombocytopenia and thrombosis (HITT); a thrombotic complication of HITT; heparin induced thrombocytopenia (HIT); vaccine-induced immune thrombotic thrombocytopenia (VITT); atherosclerosis or atherosclerotic vascular disease; decrease in platelet production; inflammation or an inflammatory disease; antiphospholipid syndrome; platelet imbalance or insufficiency; and a clotting or hemostasis disorder; or a combination thereof, in a subject, the method comprising administering to the subject a therapeutically effective amount of a compound of claim 1, or a pharmaceutically acceptable salt thereof, or a pharmaceutical composition comprising the compound.

81. The method of claim 80, wherein the disease or condition is mediated by a PF4 tetramer.

82. The method of claim 80, wherein the atherosclerosis results from a PF4 tetramer formation or a formation of a GAG-PF4 complex.

83. The method of claim 80, wherein the thrombotic complication of HITT is thrombosis.

84. The method of claim 83, wherein the thrombosis is characterized by lower than normal thrombin-antithrombin complex (TAT) level.

85.-112. (canceled)
